(19) United States
(12) Patent Application Publication
(10) Pub. No.: US 2024/0400553 A1
(43) Pub. Date: Dec. 5, 2024

(54) AMINE BASED MATRIPTASE 2 INHIBITORS AND USES THEREOF

(71) Applicant: Disc Medicine, Inc., Watertown, MA (US)

(72) Inventors: Srikanth Venkatraman, Edison, NJ (US); Vu Hong, Cambridge, MA (US); Venkateshwar Rao Gummadi, Bangalore (IN); Bharathi Raja Ainan, Hosur Taluk (IN); Brahma Reddy Vare, Karlapalem (IN); Sharad Chandrakant Deshmukh, Bangalore (IN)

(73) Assignee: Disc Medicine, Inc., Watertown, MA (US)

(21) Appl. No.: 18/632,780

(22) Filed: Apr. 11, 2024

Related U.S. Application Data
(60) Provisional application No. 63/458,772, filed on Apr. 12, 2023.

(51) Int. Cl. C07D 471/04; A61K 31/437; A61K 31/439; A61K 31/444; A61K 31/4545; A61K 31/496; A61K 31/5377; A61K 31/541; A61K 31/55; C07D 209/42; C07D 401/12; C07D 403/12; C07D 409/14; C07D 413/12; C07D 519/00

(52) U.S. Cl. CPC C07D 471/04; A61K 31/437; A61K 31/439; A61K 31/444; A61K 31/4545; A61K 31/496; A61K 31/5377; A61K 31/541; A61K 31/55; C07D 209/42; C07D 401/12; C07D 403/12; C07D 409/14; C07D 413/12; C07D 519/00

(57) ABSTRACT

The present disclosure provides amine based compounds for inhibiting matriptase 2, or a mutant thereof, and compositions and methods of use thereof.

### CROSS-REFERENCE TO RELATED APPLICATIONS

This application claims the benefit of and priority under 35 U.S.C. § 119 (e) to U.S. Provisional Application No. 63/458,772, filed Apr. 12, 2023, titled AMINE BASED MATRIPTASE 2 INHIBITORS AND USES THEREOF, the contents of which are incorporated herewith by reference in their entirety.

### TECHNICAL FIELD

The present disclosure relates to compounds, compositions, and methods useful for inhibition of Matriptase 2 (“Mat-2”), or a mutant thereof, and the treatment of various disorders. The present disclosure also provides pharmaceutical compositions comprising compounds of the present disclosure and methods of using said compositions in the treatment of various disorders.

### BACKGROUND

Matriptase-2 is a cell surface serine protease with a modular structure. Mutations in matriptase-2 cause iron-refractory iron deficiency anemia (IRIDA), an iron deficiency disorder where the level of hepcidin is inappropriately high. The enzyme activity of matriptase-2 reduces hepcidin expression through the suppression of bone morphogenetic protein (BMP)/sons of mothers against decapentaplegic homologue protein (SMAD) signaling. Loss of or inhibition of matriptase-2 activity leads to an increase in hepcidin production by the liver. Thus, there is a need to identify a Matriptase-2 modulator for the treatment of these and other conditions. The present disclosure and embodiments described herein fulfill these needs and others.

### SUMMARY OF THE EMBODIMENTS

The compounds of the present disclosure, and pharmaceutically acceptable salts thereof, are effective as Matriptase 2 inhibitors. In one aspect, the present disclosure provides compounds of Formula I:

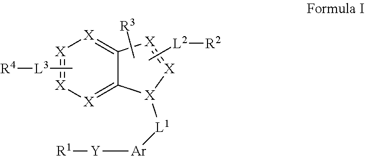

or pharmaceutically acceptable salts thereof, wherein each variable is as defined and described herein.

In some embodiments, compounds of Formula I are of Formula IV:

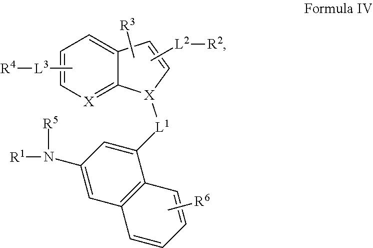

or pharmaceutically acceptable salts thereof are provided.

In some embodiments, compounds of Formula I are of Formula VIV-a:

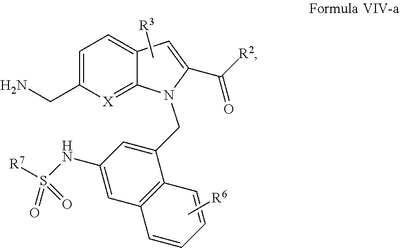

or pharmaceutically acceptable salts thereof are provided.

Also provided herein are processes for preparing the compounds disclosed herein.

The compounds of the present disclosure (e.g., compounds of Formula I, compounds as disclosed in Table A, Table B, and Table C), and pharmaceutically acceptable salts thereof, as well as the compositions disclosure herein are useful for treating a variety of diseases, disorders, and/or conditions associated with relative or absolute hepcidin deficiency, or diseases, disorders, and/or conditions in which regulating iron metabolism by increasing hepcidin production by the liver may be therapeutically useful. Such diseases, disorders, and/or conditions include those described herein. Accordingly, in another aspect, disclosed herein are methods for treating a variety of diseases, disorders, and/or conditions associated with relative or absolute hepcidin deficiency, or diseases, disorders, and/or conditions in which regulating iron metabolism by increasing hepcidin production by the liver are therapeutically useful.

### DETAILED DESCRIPTION OF CERTAIN EMBODIMENTS

Compounds of the present disclosure include those described generally herein and are further illustrated by the classes, subclasses, and species disclosed herein.

### Definitions

As used herein, the following definitions shall apply unless otherwise indicated. For purposes of the present disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell. University Science Books. Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The disclosure may be more fully appreciated by reference to the following description, including the following definitions and examples. Certain features of the disclosed compositions and methods that are described herein in the context of separate aspects may also be provided in combination in a single aspect. Alternatively, various features of the disclosed compositions and methods that are, for brevity, described in the context of a single aspect, may also be provided separately or in any subcombination.

At various places in the present specification, substituents of compounds are disclosed in groups or in ranges. It is specifically intended that the embodiments include each and every individual subcombination of the members of such groups and ranges. For example, the term “C₁₋₆ alkyl” or “C₁-C₆ alkyl” is specifically intended to individually disclose methyl, ethyl, C₃ alkyl, C₄ alkyl, C₅ alkyl, and C₆ alkyl.

It is further appreciated that certain embodiments, which are, for clarity, described in the context of separate embodiments, can also be provided in combination in a single embodiment. Conversely, various features of the embodiments, which are, for brevity, described in the context of a single embodiment, can also be provided separately or in any suitable sub-combination.

All percentages and ratios used herein, unless otherwise indicated, are by weight.

The term “alkyl,” when used alone or as part of a substituent group, refers to a straight- or branched-chain hydrocarbon group, a spirocyclic group, or a fused or bridged bicyclic group, each of which has from 1 to 12 carbon atoms (“C₁-C₁₂”), preferably 1 to 6 carbons atoms (“C₁-C₆”), in the group. In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of alkyl groups include methyl (Me, C₁alkyl), ethyl (Et, C₂alkyl), n-propyl (C₃alkyl), isopropyl (C₃alkyl), butyl (C₄alkyl), isobutyl (C₄alkyl), sec-butyl (C₄alkyl), tert-butyl (C₄alkyl), pentyl (C₅alkyl), isopentyl (C₅alkyl), tert-pentyl (C₅alkyl), hexyl (C₆alkyl), isohexyl (C₆alkyl), and the like. Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g. n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (Ca), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl. e.g., CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or f-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “spirocyclic group” refers to spirocyclic compounds in which the two rings share only one single atom, the spiro atom, which is usually a quaternary carbon. Examples of spirocyclic compounds are spiro[2,3]undecane, spiro[3,3]heptane, and spiro[5,5]undecane.

The term “fused bicyclic group” refers to fused bicyclic compounds, in which two rings share two adjacent atoms. Examples of fused bicyclic compounds include bicyclo[4.4.0]decane, α-thujene and decalin, and the like.

The term “bridged bicyclic group” refers to bridged bicyclic compounds, in which the two rings share three or more atoms, separating the two bridgehead atoms by a bridge containing at least one atom. Examples of bridged bicyclic compounds include bicyclo[2.2.1]heptane, bicyclo[1,1,1] pentane, bicyclo[2.2.1]heptane, bicyclo[2.2.2]octane, bicyclo[3.3.1]nonane, bicyclo[3.3.3]undecane, and the like.

The term “haloalkyl,” when used alone or as part of a substituent group, refers to a straight- or branched-chain hydrocarbon group having from 1 to 12 carbon atoms (“C₁-C₁₂”), preferably 1 to 6 carbons atoms (“C₁-C₆”), in the group, wherein one or more of the hydrogen atoms in the group have been replaced by a halogen atom. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include-CHF₂, —CH₂F, —CF₃, —CH₂CF₃, —CF₂CF₃, —CF₂CF₂CF₃, —CCl₃, —CFCl₂, —CF₂Cl, and the like Examples of haloalkyl groups include trifluoromethyl (—CF₃, C₁haloalkyl), trifluoroethyl (—CH₂CF₃, C₂haloalkyl), and the like. “Perhaloalkyl” is a subset of haloalkyl, and refers to an alkyl group wherein all of the hydrogen atoms are independently replaced by a halogen, e.g., fluoro, bromo, chloro, or iodo Examples of perhaloalkyl groups include trifluoromethyl (—CF₃) and pentafluoroethyl (—CF₂CF₃).

The term “lower alkyl” refers to a C₁₋₄ straight or branched alkyl group. Lower alkyl groups consist of methyl, ethyl, n-propyl, iso-propyl, n-butyl, iso-butyl, sec-butyl, and tert-butyl.

The term “halo” or “halogen” refers to chloro, fluoro, bromo, or iodo.

The term “oxo” refers to an oxygen atom (i.e., ═O) as a divalent substituent, forming a carbonyl group when attached to a carbon (e.g., C═O), or attached to a nitrogen or sulfur heteroatom forming a nitroso, sulfinyl, or sulfonyl, respectively.

The term “cycloalkyl.” “carbocyclic ring.” and “carbocyclyl” are used interchangeably, and refers to a radical of a non-aromatic cyclic hydrocarbon group having from 3 to 14 carbon atoms (“C₃-C₁₄”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (Cn), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl. In some embodiments. “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₅). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The terms “heterocycloalkyl.” “heterocyclic.” and “heterocyclyl” are used interchangeably, and refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits. In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur. Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dibydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1.4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like. In some embodiments, heterocyclyl/heterocyclic ring is imidazo[1,2-a]pyridine or pyrazolo[1,5-a]pyridine.

The term “aryl” and “aromatic carbocyclic ring” are used interchangeably throughout and refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl. Examples of aryl groups include, but are not limited to, phenyl, naphthyl, anthracenyl, phenanthrenyl, indanyl, indenyl, tetrahydronaphthyl, and the like. In some embodiments, the 8-10 membered bicyclic aromatic carbocyclic ring is tetrahydronaphthyl.

The term “heteroaryl,” “heteroaromatic.” or “heteroaromatic ring” are used interchangeably throughout and refers to a radical of a 5-14 membered monocyclic or polycyclic (e.g., bicyclic, tricyclic) 4n+2 aromatic ring system (e.g., having 6, 10, or 14 □ electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring. e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur. In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“*5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl. Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl. In some embodiments, heteroaryl/bicyclic heteroaromatic ring is quinolinyl, isoquinolinyl, 1,2-dihydrobenzo[e][1.2]azaborinine, 2-methyl-1,2-dihydrobenzo[e][1,2]azaborinine, imidazo[1,2-a]pyridine, or pyrazolo[1,5-a]pyridine. In some embodiments, heteroaryl/bicyclic heteroaromatic ring is isoquinolinyl 2-methyl-1,2-dihydrobenzo[e][1,2]azaborinine, imidazo[1,2-a]pyridine, or pyrazolo[1,5-a]pyridine.

When a range of carbon atoms is used herein, for example, C₁-C₆, all ranges, as well as individual numbers of carbon atoms, are encompassed. For example, “C₁-C₃” includes C₁-C₃, C₁-C₂, C₂-C₃, C₁, C₂, and C₃. The range of carbon atoms may be expressed with alternative expressions. For example, the term “C₁₋₆” is an alternative expression of “C₁-C₆”.

When a ring system is described herein as having a range of members, for example, “5-14-membered”, all ranges, as well as individual numbers of atoms, are encompassed. For example, “5-14-membered” includes 5-6-membered, 5-10-membered, 6-9-membered, 5-membered, 6-membered, 7-membered, 8-membered, and the like.

As used herein, “alkoxy” refers to an —O-alkyl group. Examples of alkoxy groups include methoxy, ethoxy, propoxy (e.g., n-propoxy and isopropoxy), t-butoxy, and the like.

The term “alkenyl” when used alone or as part of a substituent group refers to a straight- or branched-chain group having from 2 to 12 carbon atoms (“C₂-C₁₂”), preferably 2 to 6 carbons atoms (“C₂₋₆”), in the group, wherein the group includes at least one carbon-carbon double bond of alkenyl groups include vinyl (—CH—CH₂; C₂alkenyl), allyl (—CH₂—CH═CH₂; C₅alkenyl), propenyl (—CH═CHCH₃; C₂alkenyl); isopropenyl (—C(CH₃)=CH₂; C₅alkenyl), butenyl (—CH═CHCH₂CH₃; C₄alkenyl), sec-butenyl (—C(CH₃)=CHCH₃; C₄alkenyl), iso-butenyl (—CH═C(CH₃)₂; C₂alkenyl), 2-butenyl (—CH₂CH═CHCH₃; C₄alkyl), pentenyl (CH═CHCH₂CH₂CH₃ or CH2=CHCH₂CH₂CH₂—; C₅alkenyl), and the like. In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in I-butenyl). Examples of C₁₋₄ alkenyl groups include methylidenyl (C₁), ethenyl (C₇), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (Ca), octatrienyl (C₅), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂ alkenyl. In an alkenyl group, a C═C double bond for which the stereochemistry is not specified (e.g., —CH═CHCH₃ or

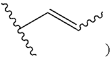

may be in the (E)- or (Z)-configuration.

The term “alkynyl” when used alone or as part of a substituent group refers to a straight- or branched-chain group having from 2 to 12 carbon atoms (“C₂-C₁₂”), preferably 2 to 6 carbons atoms (“C₂-C₆”), in the group, wherein the group includes at least one carbon-carbon triple bond. Examples of alkynyl groups include ethynyl (—C≡CH; C₂alkynyl), propargyl (—CH₂—CH≡CH; C₃alkynyl), and the like. In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₅), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₅), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for alkyl groups. Additionally, or alternatively, any substitutable nitrogen of a bridged bicyclic group is optionally substituted. Exemplary bicyclic rings include:

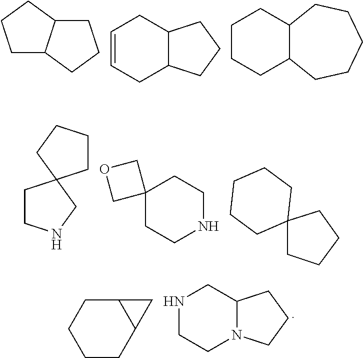

Exemplary bridged bicyclic ring systems include:

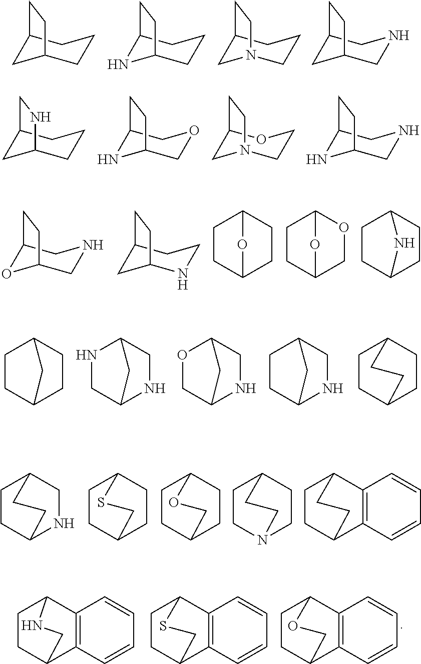

The term “heteroatom” means one or more of oxygen, sulfur, nitrogen, phosphorus, or silicon (including any oxidized form of nitrogen, sulfur, phosphorus, or silicon: the quaternized form of any basic nitrogen or; substitutable nitrogen of a heterocyclic ring, for example, N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl. Each group may be optionally substituted.

As used herein, the term “bivalent C₁₋₈ (or C₁₋₆) saturated or unsaturated, straight or branched, hydrocarbon chain” refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted alkyl group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted alkyl group.

The term “alkynylene” refers to a bivalent alkynyl group. A substituted alkynylene chain is a polymethylene group containing at least one triple bond in which one or more hydrogen atoms are replaced with a substituent. Suitable substituents include those described below for a substituted alkyl group.

The term “halogen” or “halo” means F, Cl, Br. or I.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the present disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by the present disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. As used herein, the phrase “suitable substituent” or “substituent” means a group that does not nullify the synthetic or pharmaceutical utility of the compounds described herein or the intermediates useful for preparing them. Examples of suitable substituents include, but are not limited to C₁-C₆ alkyl, C₁-C₆ alkenyl, C₁-C₆ alkynyl, C₅-C₆ aryl, C₁-C₆ alkoxy, C₃-C₅ heteroaryl, C₃-C₆ cycloalkyl, C₅-C₆ aryloxy, —CN, —OH, oxo, halo, haloalkyl, —NO₂, —CO₂H, —NH₂, —NH(C₁-C₈ alkyl), —N(C₁-C₈ alkyl)₂, —NH(C₆ aryl), —N(C₅-C₆ aryl)₂, —CHO, —CO(C₁-C₆ alkyl), —CO((C₅-C₆) aryl), —CO₂((C₁-C₆) alkyl), and —CO₂((C₅-C₆) aryl). One of skill in art can readily choose a suitable substituent based on the stability and pharmacological and synthetic activity of the compounds described herein.

In some embodiments, an optional substituent on a substitutable carbon is a monovalent substituent independently selected from halogen, —(CH₂)₀₋₄R^{a}, —(CH₂)₀₋₄OR^{a}, —O(CH₂)₀₋₄R^{a}, —O—(CH₂)₀₋₄C(O)OR^{a}, —(CH₂)₀₋₄CH(OR^{a})₂, —(CH₂)₀₋₄SR^{a}, —(CH₂)₀₋₄Ph, which may be substituted with R^{a}, —(CH₂)₀₋₄O(CH₂)₀₋₁Ph which may be substituted with R^{a}, —CH═CHPh, which may be substituted with R^{a}, —(CH₂)₀₋₄O(CH₂)₀₋₁-pyridyl which may be substituted with R^{a}, —NO₂, —CN, —N₃, —(CH₂)₀₋₄N(R^{a})₂, —(CH₂)₀₋₄N(R^{a})C(O)R^{a}, —N(R^{a})C(S)R^{a}, —(CH₂)₀₋₄N(R^{a})C(O)NR^{a}₂, —N(R^{a})C(S)NR^{a}₂, —(CH₂)₀₋₄N(R^{a})C(O)OR^{a}, —N(R^{a})N(R^{a})C(O)R^{a}, —N(R^{a})N(R^{a})C(O)NR^{a}₂, —N(R^{a})N(R^{a})C(O)OR^{a}, —(CH₂)₀₋₄C(O)R^{a}, —C(S)R^{a}, —(CH₂)₀₋₄C(O)OR^{a}, —(CH₂)₀₋₄C(O)SR^{a}, —(CH₂)₀₋₄C(O)OSiR^{a}₃, —(CH₂)₀₋₄OC(O)R^{a}, —OC(O)(CH₂)₀₋₄SR—, SC(S)SR^{a}, —(CH₂)₀₋₄SC(O)R^{a}, —(CH₂)₀₋₄C(O)NR^{a}₂, —C(S)NR^{a}₂, —C(S)SR^{a}, —SC(S)SR^{a}, —(CH₂)₀₋₄OC(O)NR^{a}₂, —C(O)N(OR^{a})R^{a}, —C(O)C(O)R^{a}, —C(O)CH₂C(O)R^{a}, —C(NOR^{a})R^{a}, —(CH₂)₀₋₄SSR^{a}, —(CH₂)₀₋₄S(O)₂R^{a}, —(CH₂)₀₋₄S(O)₂OR^{a}, —(CH₂)₀₋₄OS(O)₂R^{a}, —S(O)₂NR^{a}₂, —S(O)(NR^{a})R^{a}, —S(O)₂N=C(NR^{a}₂)₂, —(CH₂)₀₋₄ S(O)R^{a}, —N(R^{a})S(O)₂NR^{a}₂, —N(R^{a})S(O)₂R^{a}, —N(OR^{a})R^{a}, —C(NH)NR^{a}₂, —P(O)₂R^{a}, —P(O)R^{a}2, —OP(O)R^{a}₂, —OP(O)(OR^{a})₂, SiR^{a}₃, —(C₁₋₄ straight or branched alkylene)O—N(R^{a})₂, or —(C₁₋₄ straight or branched alkylene)C(O)O—N(R^{a})₂.

In some embodiments, each R^{a} is independently hydrogen, C₁₋₆ alkyl, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R selected from ═O and ═S; or each R is optionally substituted with a monovalent substituent independently selected from halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, (CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂ NHR^{●}, —(CH₂)₀₋₂NR^{●}₂, —NO₂, —SIR^{●}₃, —OSIR^{●}₃, —C(O)SR^{●}, —(C₁₋₄ straight or branched alkylene)C(O)OR^{●}, or —SSR^{●}.

In some embodiments, each R^{●} is independently selected from C₁₋₄ alkyl, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by a halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

In some embodiments, when R* is C₁₋₆ alkyl, R* is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ alkyl, —CH₂Ph. —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

In some embodiments, an optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ alkyl, unsubstituted-OPh, or an unsubstituted 5-6-membered saturated, partially unsaturated, or aryl ring having 0-4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ alkyl, R^{†} is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ alkyl, —CH₂Ph, —O(CH₂)₀₋₁Ph. or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al. describe pharmaceutically acceptable salts in detail in J. Pharmaceutical Sciences, 1977, 66, 1-19, incorporated herein by reference. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counter ions such as halide, hydroxide, carboxylate, sulfate, phosphate, nitrate, lower alkyl sulfonate and aryl sulfonate.

Unless otherwise stated, structures depicted herein are also meant to include all enantiomeric, diastereomeric, and geometric (or conformational) forms of the structure: for example, the R and S configurations for each asymmetric center. Z and E double bond isomers, and Z and E conformational isomers. Therefore, single stereochemical isomers as well as enantiomeric, diastereomeric, and geometric (or conformational) mixtures of the present compounds are within the scope of the present disclosure. Unless otherwise stated, all tautomeric forms of the compounds of the present disclosure are within the scope of the present disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of the present disclosure. Such compounds are useful, for example, as analytical tools, as probes in biological assays, or as therapeutic agents in accordance with the present disclosure. In certain embodiments, a compound of the present disclosure comprises one or more deuterium atoms.

As used herein, the terms “inhibitor,” “inhibition,” “inhibiting,” “inhibit,” and the like are defined as a compound that binds to and/or inhibits Matriptase 2, or a mutant thereof, with measurable affinity. In certain embodiments, an inhibitor has an IC₅₀ and/or binding constant (Kᵢ) of less than about 100 μM, less than about 50 μM, less than about 20 μM, less than about 10 μM. or less than about 5 μM. In certain embodiments, an inhibitor has a binding constant (Kᵢ) of less than about 100 μM, less than about 50 μM, less than about 20 μM, less than about 10 UM, or less than about 5 μM. In some embodiments, Kᵢ is lower than IC₅₀. In some embodiments, IC₅₀ is the same as Kᵢ. In some embodiments, IC₅₀ is the same as Kᵢ for noncompetitive inhibition.

As used herein the term “inhibit” or “inhibition” in the context of enzymes, for example, in the context of Matriptase 2, or mutant/variant thereof, refers to a reduction in the activity of the enzyme. In some embodiments, the term refers to a reduction of the level of enzyme activity. e.g., Matriptase 2, or mutant/variant thereof, activity, to a level that is statistically significantly lower than an initial level, which may, for example, be a baseline level of enzyme activity. In some embodiments, the term refers to a reduction of the level of enzyme activity, e.g., Matriptase 2, or mutant/variant thereof, activity, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of enzyme activity.

The terms “measurable affinity” and “measurably inhibit,” as used herein, means a measurable change in Matriptase 2, or a mutant thereof, activity between a sample comprising a compound of the present disclosure, or composition thereof, and Matriptase 2, or a mutant thereof, and an equivalent sample comprising Matriptase 2, or a mutant thereof, in the absence of said compound, or composition thereof.

The term “neurodegenerative disease” refers to a type of neurological disease marked by the loss of nerve cells, including, but not limited to, Alzheimer's disease, Parkinson's disease, amyotrophic lateral sclerosis, tauopathies (including frontotemporal dementia), and Huntington's disease.

The term “liver disease” or “hepatic disease” refers to damage to or a disease of the liver. In some embodiments, the liver disease is selected from the group consisting of Hepatitis B, Hepatitis C, alcoholic liver disease, cirrhosis of the liver, epahtocellular carcinoma, and non-alcoholic steatohepatitis (NASH).

The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. In some embodiments, the metabolic disease is selected from the group consisting of metabolic syndrome, insulin resistance, Type I diabetes, Type II diabetes, gestational diabetes, hyperglycemia, hyperinsulinemia, obesity, porphyria, porphyria cutanea tarda, Wilson's Disease, and acute iron overdose. In some embodiments, the metabolic disease is selected from the group consisting of metabolic syndrome, insulin resistance. Type II diabetes, porphyria, porphyria cutanea tarda, Wilson's Disease, and acute iron overdose.

A “hematological disease” includes a disease which affects a hematopoietic cell or tissue. Hematological diseases include diseases associated with aberrant hematological content and/or function. In some embodiments, the hematological disease, disorder, and/or condition is selected from the group consisting of sickle cell disease (such as sickle cell anemia), polycythemia vera, sideroblastic anemia, and bone marrow transplantation.

The terms “infectious disease” and “infectious disorder” refer to diseases and disorders caused by microorganisms, for example bacteria viruses, fungi, or parasite. In some embodiments, the infectious is a siderophilic infection.

The terms “disease.” “disorder,” and “condition” are used interchangeably throughout.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the present disclosure are administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

### Compounds

Compounds of the present disclosure, and pharmaceutical compositions thereof, are useful as inhibitors of Matriptase 2, or a mutant thereof. Without wishing to be bound by any particular theory, it is believed that compounds of the present disclosure, and pharmaceutical compositions thereof, may inhibit the activity of Matriptase 2, or a mutant thereof, and thus treat certain diseases, disorders, or conditions associated with relative or absolute hepcidin deficiency, or diseases, disorders, or conditions in which regulating iron metabolism by increasing hepcidin production by the liver may be therapeutically useful, such as those described herein.

It has now been found that compounds of the present disclosure, and pharmaceutical compositions thereof, are effective as Matriptase 2 inhibitors.

In one aspect, the present disclosure provides a compound of Formula I′:

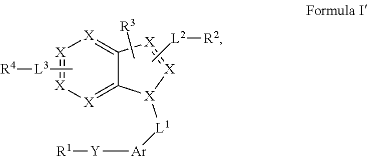

or a pharmaceutically acceptable salt thereof,
wherein:
Ar is an optionally substituted ring selected from aryl, heteroaryl, carbocyclyl, and heterocyclyl;
each X is independently CH, C-L³-R⁴, C-L²-R², C—R³, or N;
Y is a bond, —NR⁵—, CR¹⁰R^{10′}, or —O—;
L¹ is a bond, or an optionally substituted bivalent C₁₋₈ saturated or unsaturated, straight or branched hydrocarbon chain, wherein 1, 2, or 3 methylene units of the hydrocarbon chain are optionally and independently replaced by —S(O)₂—, —C(O)—, —NH—, or —O—;
R¹ is H, D, R⁷, R⁷SO₂—, R⁷S(O)—, R⁷C(O)—, R⁷NR—C(O)—, or R⁷O—C(O)—;
R⁷ is R¹¹(CR¹⁰OR^{10′})ₙ—;
R¹¹ is H, an optionally substituted C₁₋₆ alkyl, or an optionally substituted ring selected from aryl, heteroaryl, carbocyclyl, and heterocyclyl;
R⁵, R¹⁰, and R^{10′} are each, independently, H, D, halo, oxo, C₁₋₆ alkyl, C₂₋₆ alkenyl, C₂₋₆ alkynyl, C₁₋₆ haloalkyl, C₃₋₆ cycloalkyl, CN, NO₂, C(O)NR₂, C(O)OR, S(O)OR, SO₂OR, S(O)NR₂, SO₂NR₂, or B(OR)₂;
optionally, R¹⁰ and R^{10′} together with the carbon atom to which they are both attached form an optionally substituted C₃₋₇ spirocyclic ring;
n is 0-8;
L² is an optionally substituted bivalent C₁₋₈ saturated or unsaturated, straight or branched hydrocarbon chain, wherein 1, 2, or 3 methylene units of the hydrocarbon chain are optionally and independently replaced by —NR—C(O)—, —C(O)—NR—, —C(O)—, —S(O)₂—, —C(O)—O—, —O—C(O)—, —NR—S(O)₂—, —S(O)₂—NR—, or -Cy-;
-Cy- is an optionally substituted bivalent ring selected from aryl, heteroaryl, heterocyclyl, and carbocyclyl;
R² is R¹⁴(CR¹⁵R^{15′})ₚ—;

R¹⁴ is H, D, —OH, —NR₂, —O(C₁₋₆ alkyl), —O(C₁₋₆ alkyl)-(optionally substituted 4-7 heterocyclyl), an optionally substituted C₁₋₆ alkyl, or an optionally substituted ring selected from carbocyclyl, heterocyclyl, heteroaryl, and aryl;
R¹⁵, and R^{15′} are each, independently, H, D, halo, oxo, C₁₋₆ alkyl, C₂₋₆ alkenyl, C₂₋₆ alkynyl, C₁₋₆ haloalkyl, C₃₋₆ cycloalkyl, CN, NO₂, C(O)NR₂, C(O)OR, or B(OR)₂;
p is 0-8;
R³ is absent, hydrogen, —OH, halogen, —CN, —C(O) H, —NH₂, —NO₂, —COOH, —CONH₂, —NH—C(O)—O—C₁₋₆ alkyl, C₁₋₆ alkyl, or —C(O)—C₁₋₆ alkyl, wherein the C₁₋₆ alkyl is optionally substituted;
L³ is a bond, or an optionally substituted bivalent C₁₋₈ saturated or unsaturated, straight or branched hydrocarbon chain, wherein 1, 2, or 3 methylene units of the hydrocarbon chain are optionally replaced by —NR—C(O)—, —C(O)—NR—, —C(O)—, —S(O)₂—, —C(O)—O—, —O—C(O)—, —NR—S(O)₂—, or —S(O)₂—NR—;
R⁴ is —NH₂, NHR, —N═C═N—, —NR₂, —CH₂—NH—C≡N, —PO₄H₂, or —H; and
each R is, independently, H, —OH, —C₁₋₈ alkyl, —C₁₋₈ alkyl(carbocyclyl), —OC₁₋₈ alkyl, —C(O)—C₁₋₈ alkyl, —O—C(O)—C₁₋₈ alkyl, C₁₋₈ alkyl-O—C(O)—C₁₋₈ alkyl, —C(O)—OC₁₋₈ alkyl, 3-7 carbocyclyl, —O-(carbocyclyl), —C(O)-(carbocyclyl), —C(O)—O-(carbocyclyl), aryl, —O-aryl, —C(O)-aryl, —C(O)—O-aryl, —C(O)-aryl-(C₁₋₈ alkyl)-O—C(O)—O—(C₁₋₈ alkyl)-, —C(O)—O—(C₁₋₈ alkyl)-(heterocyclyl), —O-(aryl), —C(O)-(aryl), or —C(O)—O-(aryl), wherein each of the C₁₋₈ alkyl, carbocyclyl, aryl, and heterocyclyl is optionally and independently substituted,
with the proviso that the compound is not

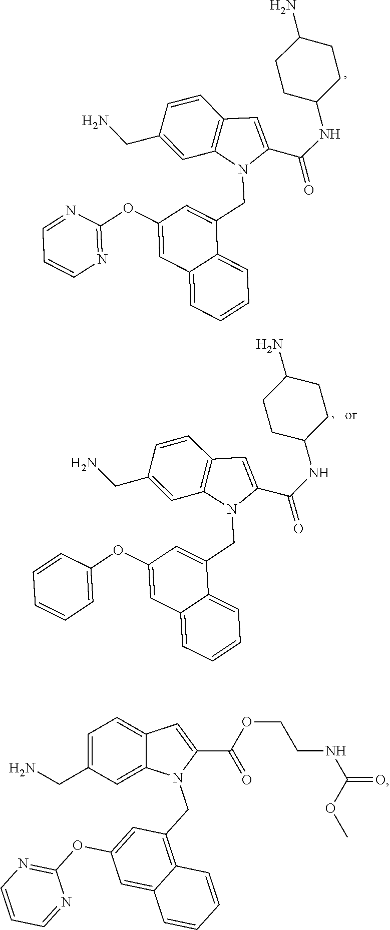 [SMILES: NCc1ccc2cc(C(=O)NC3CCC(N)CC3)n(Cc3cc(Oc4ncccn4)cc4ccccc34)c2c1 ; NCc1ccc2cc(C(=O)NC3CCC(N)CC3)n(Cc3cc(Oc4ccccc4)cc4ccccc34)c2c1 ; COC(=O)NCCOC(=O)c1cc2ccc(CN)cc2n1Cc1cc(Oc2ncccn2)cc2ccccc12]

or a pharmaceutically acceptable salt thereof. In some embodiments the compound of Formula I′ is of Formula I.

In one aspect, the present disclosure provides a compound of Formula I:

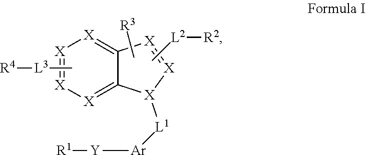

or a pharmaceutically acceptable salt thereof.
wherein:
Ar is an optionally substituted ring selected from phenyl, a 5-6 membered heteroaromatic ring having 1-4 heteroatoms independently selected from nitrogen, oxygen, and sulfur, an 8-10 membered bicyclic aromatic carbocyclic ring, and an 8-10 membered bicyclic heteroaromatic ring having 1-4 heteroatoms independently selected from nitrogen, oxygen, and sulfur;
each X is independently CH, C-L³-R⁴, C-L²-R², C—R³, or N;
Y is a bond, —NR⁵—, CR¹⁰R^{10′}, or —O—;
L¹ is a bond, or an optionally substituted bivalent C₁₋₈ saturated or unsaturated, straight or branched hydrocarbon chain, wherein 1, 2, or 3 methylene units of the hydrocarbon chain are optionally and independently replaced by —S(O)₂—, —C(O)—, —NH—, or —O—;
R¹ is H, D, R⁷, R⁷SO₂—, R⁷S(O)—, R⁷C(O)—, R⁷NR—C(O)—, or R⁷O—C(O)—;
R⁷ is R¹¹(CR¹⁰R^{10′})ₙ—;
R¹¹ is H, an optionally substituted C₁₋₆ alkyl, or an optionally substituted ring selected from phenyl, a 5-6 membered heteroaromatic ring having 1-4 heteroatoms independently selected from nitrogen, oxygen, and sulfur, a 3-7 membered monocyclic carbocyclic ring, a 4-7 membered heterocyclic ring having 1-3 heteroatoms independently selected from nitrogen, oxygen, and sulfur, a 5-10 membered bicyclic carbocyclic ring, a 7-10 membered bicyclic heterocyclic ring having 1-4 heteroatoms independently selected from nitrogen, oxygen, and sulfur, an 8-10 membered bicyclic aromatic carbocyclic ring, and an 8-10 membered bicyclic heterocyclic ring having 1-3 heteroatoms independently selected from nitrogen, oxygen, and sulfur;
R⁵, R¹⁰, and R^{10′} are each, independently, H, D, halo, oxo, C₁₋₆ alkyl, C₂₋₆ alkenyl, C₂₋₆ alkynyl, C₁₋₆ haloalkyl, C₃₋₆ cycloalkyl, CN, NO₂, C(O)NR₂, C(O)OR, S(O)OR, SO₂OR, S(O)NR₂, SO₂NR₂, or B(OR)₂;
optionally, R¹⁰ and R^{10′} together with the carbon atom to which they are both attached form an optionally substituted C₃₋₇ spirocyclic ring;
n is 0-8;
L² is an optionally substituted bivalent C₁₋₈ saturated or unsaturated, straight or branched hydrocarbon chain, wherein 1, 2, or 3 methylene units of the hydrocarbon chain are optionally and independently replaced by —NR—C(O)—, —C(O)—NR—, —C(O)—, —S(O)₂—, —C(O)—O—, —O—C(O)—, —NR—S(O)₂—, —S(O)₂—NR—, or -Cy-;
-Cy- is an optionally substituted bivalent ring selected from phenyl, a 4-6 membered monocyclic heterocyclic ring having 1-4 heteroatoms independently selected from nitrogen, oxygen, and sulfur, a 5-10 membered bicyclic carbocyclic ring, and a 4-6 membered monocyclic heteroaromatic ring having 1-4 heteroatoms independently selected from nitrogen, oxygen, and sulfur;
R² is R¹⁴ (CR¹⁵R^{15′})ₚ—;
R¹⁴ is H, D, —OH, —NR₂, —O(C₁₋₆ alkyl), —O(C₁₋₆ alkyl)-(optionally substituted 4-7 membered monocyclic heterocyclic ring), an optionally substituted C₁₋₆ alkyl, or an optionally substituted ring selected from a 3-7 membered monocyclic carbocyclic ring, a 4-7 membered monocyclic heterocyclic ring having 1-4 heteroatoms independently selected from nitrogen, oxygen, and sulfur, a 5-10 membered bicyclic carbocyclic ring, a 7-10 membered bicyclic heterocyclic ring having 1-4 heteroatoms independently selected from nitrogen, oxygen, and sulfur, phenyl, 5-6 membered monocyclic heteroaromatic ring having 1-4 heteroatoms independently selected from nitrogen, oxygen, and sulfur, a 8-10 membered bicyclic aromatic ring, a 8-10 membered bicyclic heteroaromatic ring having 1-4 heteroatoms independently selected from nitrogen, oxygen, and sulfur, and adamantyl;
R¹⁵, and R^{15′} are each, independently. H, D, halo, oxo, C₁₋₆ alkyl, C₂₋₆ alkenyl, C₂₋₆ alkynyl, C₁₋₆ haloalkyl, C₃₋₆ cycloalkyl, CN, NO₂, C(O)NR₂, C(O)OR, or B(OR)₂;
p is 0-8;
R³ is absent, hydrogen, —OH, halogen, —CN, —C(O) H, —NH₂, —NO₂, —COOH, —CONH₂, —NH—C(O)—O—C₁₋₆ alkyl, C₁₋₆ alkyl, or —C(O)—C₁₋₆ alkyl, wherein the C₁₋₆ alkyl is optionally substituted;
L³ is a bond, or an optionally substituted bivalent C₁₋₈ saturated or unsaturated, straight or branched hydrocarbon chain, wherein 1, 2, or 3 methylene units of the hydrocarbon chain are optionally replaced by —NR—C(O)—, —C(O)—NR—, —C(O)—, —S(O)₂—, —C(O)—O—, —O—C(O)—, —NR—S(O)₂—, or —S(O)₂—NR—;

R⁴ is —NH₂. NHR, —N═C═N—, —NR₂, —CH₂—NH—C═N, —PO₄H₂, or —H. and
each R is, independently, H, —OH, —C₁₋₈ alkyl, —C₁₋₈ alkyl(3-7 membered monocyclic carbocyclyl), —OC₁₋₈ alkyl, —C(O)—C₁₋₈ alkyl, —O—C(O)—C₁₋₈ alkyl, C₁₋₈ alkyl-O—C(O)—C₁₋₈ alkyl, —C(O)—OC₁₋₈ alkyl, 3-7 membered monocyclic carbocyclyl, —O-(4-7 membered monocyclic carbocyclyl), —C(O)-(4-7 membered monocyclic carbocyclyl), —C(O)—O-(4-7 membered monocyclic carbocyclyl), phenyl, —O-phenyl, —C(O)-phenyl, —C(O)—O-phenyl, —C(O)-phenyl-(C₁₋₈ alkyl)-O—C(O)—O—(C₁₋₈ alkyl)-, —C(O)—O—(C₁₋₈ alkyl)-(4-7 membered monocyclic heterocyclic ring), 8-10 membered bicyclic aryl, —O-(8-10 membered bicyclic aryl), —C(O)-(8-10 membered bicyclic aryl), or —C(O)—O-(8-10 membered bicyclic aryl), wherein each of the C₁₋₈ alkyl, 4-7 membered monocyclic carbocyclyl, phenyl, and 8-10 membered bicyclic aryl is optionally and independently substituted,
with the proviso that the compound is not

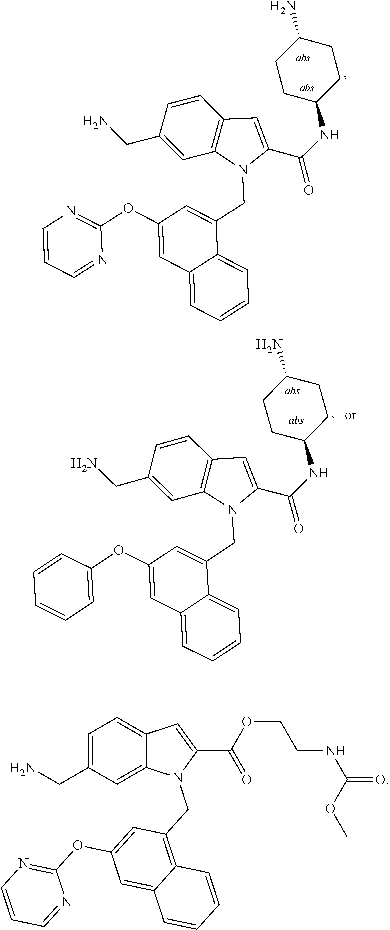 [SMILES: NCc1ccc2cc(C(=O)N[C@H]3CC[C@H](N)CC3)n(Cc3cc(Oc4ncccn4)cc4ccccc34)c2c1 ; NCc1ccc2cc(C(=O)N[C@H]3CC[C@H](N)CC3)n(Cc3cc(Oc4ccccc4)cc4ccccc34)c2c1 ; COC(=O)NCCOC(=O)c1cc2ccc(CN)cc2n1Cc1cc(Oc2ncccn2)cc2ccccc12]

In some embodiments, the present disclosure provides a compound of Formula I:

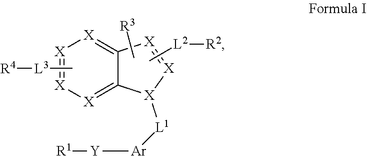

or a pharmaceutically acceptable salt thereof,
wherein:
Ar is an optionally substituted ring selected from phenyl, a 5-6 membered heteroaromatic ring having 1-4 heteroatoms independently selected from nitrogen, oxygen, and sulfur, an 8-10 membered bicyclic aromatic carbocyclic ring, and an 8-10 membered bicyclic heteroaromatic ring having 1-4 heteroatoms independently selected from nitrogen, oxygen, and sulfur;
each X is independently CH, C-L³-R⁴, C-L²-R², C—R³, or N;
Y is a bond, —NR⁵—, CR¹⁰R^{10′} or —O—;
L¹ is a bond, or an optionally substituted bivalent C₁₋₈ saturated or unsaturated, straight or branched hydrocarbon chain, wherein 1, 2, or 3 methylene units of the hydrocarbon chain are optionally and independently replaced by —S(O)₂—, —C(O)—, —NH—, or —O—;
R¹ is H, D, R⁷, R⁷SO₂—, R⁷S(O)—, R⁷C(O)—, R⁷NR—C(O)—, or R⁷O—C(O)—;
R⁷ is R¹¹(CR¹⁰R^{10′})ₙ—;
R¹¹ is H, an optionally substituted C₁₋₆ alkyl, or an optionally substituted ring selected from phenyl, a 5-6 membered heteroaromatic ring having 1-4 heteroatoms independently selected from nitrogen, oxygen, and sulfur, a 3-7 membered monocyclic carbocyclic ring, a 4-7 membered heterocyclic ring having 1-3 heteroatoms independently selected from nitrogen, oxygen, and sulfur, a 5-10 membered bicyclic carbocyclic ring, a 7-10 membered bicyclic heterocyclic ring having 1-4 heteroatoms independently selected from nitrogen, oxygen, and sulfur, an 8-10 membered bicyclic aromatic carbocyclic ring, and an 8-10 membered bicyclic heterocyclic ring having 1-3 heteroatoms independently selected from nitrogen, oxygen, and sulfur;
R⁵, R¹⁰, and R^{10′} are each, independently. H, D, halo, oxo, C₁₋₆ alkyl, C₂₋₆ alkenyl, C₂₋₆ alkynyl, C₁₋₆ haloalkyl, C₃₋₆ cycloalkyl, CN, NO₂, C(O)NR₂, C(O)OR, S(O)OR, SO₂OR, S(O)NR₂, SO₂NR₂, or B(OR)₂;
optionally, R¹⁰ and R^{10′} together with the carbon atom to which they are both attached form an optionally substituted C₃₋₇ spirocyclic ring;
n is 0-8;
L² is an optionally substituted bivalent C₁₋₈ saturated or unsaturated, straight or branched hydrocarbon chain, wherein 1, 2, or 3 methylene units of the hydrocarbon chain are optionally and independently replaced by —NR—C(O)—, —C(O)—NR—, —C(O)—, —S(O)₂—, —C(O)—O—, —O—C(O)—, —NR—S(O)₂—, —S(O)₂—NR—, or -Cy-;
Cy- is an optionally substituted bivalent ring selected from phenyl, a 4-6 membered monocyclic heterocyclic ring having 1-4 heteroatoms independently selected from nitrogen, oxygen, and sulfur, a 5-10 membered bicyclic carbocyclic ring, and a 4-6 membered monocyclic heteroaromatic ring having 1-4 heteroatoms independently selected from nitrogen, oxygen, and sulfur;
R² is R¹⁴(CR¹⁵R^{15′})ₚ—;
R¹⁴ is H, D, —OH, —NR₂, —O(C₁₋₆ alkyl), —((C₁₋₆ alkyl)-(optionally substituted 4-7 membered monocyclic heterocyclic ring), an optionally substituted C₁₋₆ alkyl, or an optionally substituted ring selected from a 3-7 membered monocyclic carbocyclic ring, a 4-7 membered monocyclic heterocyclic ring having 1-4 heteroatoms independently selected from nitrogen, oxygen, and sulfur, a 5-10 membered bicyclic carbocyclic ring, a 7-10 membered bicyclic heterocyclic ring having 1-4 heteroatoms independently selected from nitrogen, oxygen, and sulfur, phenyl, 5-6 membered monocyclic heteroaromatic ring having 1-4 heteroatoms independently selected from nitrogen, oxygen, and sulfur, a 8-10 membered bicyclic aromatic ring, a 8-10 membered bicyclic heteroaromatic ring having 1-4 heteroatoms independently selected from nitrogen, oxygen, and sulfur, and adamantyl;
R¹⁵, and R^{15′} are each, independently, H, D, halo, oxo, C₁₋₆ alkyl, C₂₋₆ alkenyl, C₂₋₆ alkynyl, C₁₋₆ haloalkyl, C₃₋₆ cycloalkyl, CN, NO₂, C(O)NR₂, C(O)OR, or B(OR)₂;
p is 0-8;
R³ is absent, hydrogen, —OH, halogen, —CN, —C(O) H, —NH₂, —NO₂, —COOH, —CONH₂, —NH—C(O)—O—C₁₋₆ alkyl, C₁₋₆ alkyl, or —C(O)—C₁₋₆ alkyl, wherein the C₁₋₆ alkyl is optionally substituted;
L³ is a bond, or an optionally substituted bivalent C′1-8 saturated or unsaturated, straight or branched hydrocarbon chain, wherein 1, 2, or 3 methylene units of the hydrocarbon chain are optionally replaced by —NR—C(O)—, —C(O)—NR—, —C(O)—, —S(O)₂—, —C(O)—O—, —O—C(O)—, —NR—S(O)₂—, or —S(O)₂—NR—;
R⁴ is —NH₂. NHR, —N═C═N—, —NR₂, —CH₂—NH—C═N, —PO₄H₂, or —H. and
each R is, independently, H, —OH, —C₁₋₈ alkyl, —C₁₋₈ alkyl(3-7 membered monocyclic carbocyclyl), —OC₁₋₈ alkyl, —C(O)—C₁₋₈ alkyl, —O—C(O)—C₁₋₈ alkyl, C₁₋₈ alkyl-O—C(O)—C₁₋₈ alkyl, —C(O)—OC₁₋₈ alkyl, 3-7 membered monocyclic carbocyclyl, —O-(4-7 membered monocyclic carbocyclyl), —C(O)-(4-7 membered monocyclic carbocyclyl), —C(O)—O-(4-7 membered monocyclic carbocyclyl), phenyl, —O-phenyl, —C(O)-phenyl, —C(O)—O-phenyl, —C(O)-phenyl-(C₁₋₈ alkyl)-O—C(O)—O—(C₁₋₈ alkyl)-, —C(O)—O—(C₁₋₈ alkyl)-(4-7 membered monocyclic heterocyclic ring), 8-10 membered bicyclic aryl, —O-(8-10 membered bicyclic aryl), —C(O))-(8-10 membered bicyclic aryl), or —C(O)—O-(8-10 membered bicyclic aryl), wherein each of the C₁₋₈ alkyl, 4-7 membered monocyclic carbocyclyl, phenyl, and 8-10 membered bicyclic aryl is optionally and independently substituted,
with the proviso that the compound is not

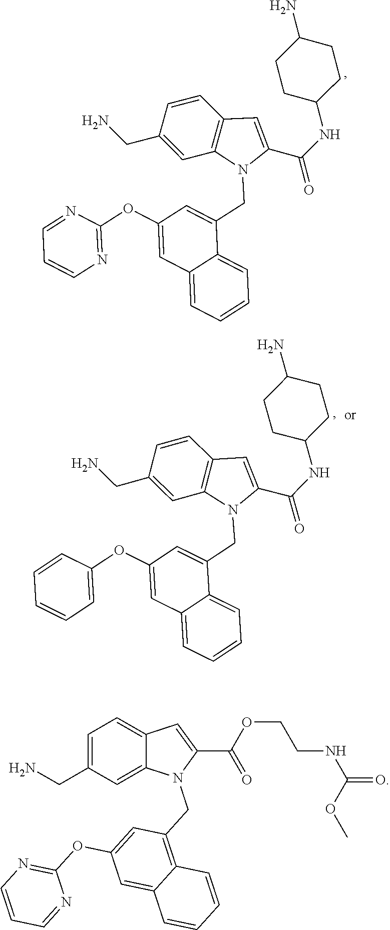 [SMILES: NCc1ccc2cc(C(=O)NC3CCC(N)CC3)n(Cc3cc(Oc4ncccn4)cc4ccccc34)c2c1 ; NCc1ccc2cc(C(=O)NC3CCC(N)CC3)n(Cc3cc(Oc4ccccc4)cc4ccccc34)c2c1 ; COC(=O)NCCOC(=O)c1cc2ccc(CN)cc2n1Cc1cc(Oc2ncccn2)cc2ccccc12]

In some embodiments, the present disclosure provides a compound of:

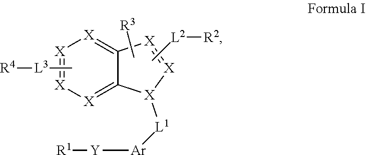

or pharmaceutically acceptable salts thereof,
wherein:
Ar is an optionally substituted ring selected from phenyl, a 5-6 membered heteroaromatic ring having 1-4 heteroatoms independently selected from nitrogen, oxygen, and sulfur, an 8-10 membered bicyclic aromatic carbocyclic ring, and an 8-10 membered bicyclic heteroaromatic ring having 1-4 heteroatoms independently selected from nitrogen, oxygen, and sulfur;
each X is independently CH or N;
Y is —NR⁵—, CR¹⁰R^{10′}, or —O—;
L¹ is a bond, or an optionally substituted bivalent C₁₋₈ saturated or unsaturated, straight or branched hydrocarbon chain, wherein 1, 2, or 3 methylene units of the hydrocarbon chain are optionally and independently replaced by —S(O)₂—, —C(O)—, —NH—;
R¹ is H, D, R⁷, R⁷SO₂—, R⁷S(O)—, R⁷C(O)—, or R⁷O—C(O)—;
R⁷ is R¹¹(CR¹⁰R^{10′})ₙ—;
R¹¹ is H, an optionally substituted C₁₋₆ alkyl, or an optionally substituted ring selected from phenyl, a 5-7 membered heterocyclic ring having 1-3 heteroatoms independently selected from nitrogen, oxygen, and sulfur, an 8-10 membered bicyclic aromatic carbocyclic ring, and an 8-10 membered bicyclic heterocyclic ring having 1-3 heteroatoms independently selected from nitrogen, oxygen, and sulfur;
R⁵, R¹⁰, and R^{10′} are each, independently, H, D, halo, oxo, C₁₋₆ alkyl, C₂₋₆ alkenyl, C₂₋₆ alkynyl, C₁₋₆ haloalkyl, C₃₋₆ cycloalkyl, CN, NO₂, C(O)NR₂, C(O)OR, or B(OR)₂;
optionally, R¹⁰ and R^{10′} together with the carbon atom to which they are both attached form an optionally substituted C₃₋₇ spirocyclic ring;

n is 0-8;
L² is an optionally substituted bivalent C₁₋₈ saturated or unsaturated, straight or branched hydrocarbon chain, wherein 1, 2, or 3 methylene units of the hydrocarbon chain are optionally and independently replaced by —NR—C(O)—, —C(O)—NR—, —C(O)—, —S(O)₂—, —C(O)—O—, —O—C(O)—, —NR—S(O)₂—, —S(O)₂—NR—, or -Cy-,
-Cy- is an optionally substituted bivalent ring selected from phenyl, a 4-6 membered monocyclic heterocyclic ring having 1-4 heteroatoms independently selected from nitrogen, oxygen, and sulfur, and a 4-6 membered monocyclic heteroaromatic ring having 1-4 heteroatoms independently selected from nitrogen, oxygen, and sulfur;
R² is R¹⁴ (CR¹⁵R^{15′})ₚ—;
R¹⁴ is H, D, an optionally substituted C₁₋₆ alkyl, or an optionally substituted ring selected from a 4-7 membered monocyclic carbocyclic ring, a 4-7 membered monocyclic heterocyclic ring having 1-4 heteroatoms independently selected from nitrogen, oxygen, and sulfur, a 7-10 membered bicyclic carbocyclic ring, a 7-10 membered bicyclic heterocyclic ring having 1-4 heteroatoms independently selected from nitrogen, oxygen, and sulfur, phenyl, 5-6 membered monocyclic heteroaromatic ring having 1-4 heteroatoms independently selected from nitrogen, oxygen, and sulfur, an 8-10 membered bicyclic aromatic ring, an 8-10 membered bicyclic heteroaromatic ring having 1-4 heteroatoms independently selected from nitrogen, oxygen, and sulfur, and adamantyl;
R¹⁵, and R^{15′} are each, independently. H, D, halo, oxo, C₁₋₆ alkyl, C₂₋₆ alkenyl, C₂₋₆ alkynyl, C₁₋₆ haloalkyl, C₃₋₆ cycloalkyl, CN, NO₂, C(O)NR₂, C(O)OR, or B(OR)₂;
p is 0-8;
R³ is absent, —OH, halogen, —CN, —C(O) H, —NH₂, —NO₂, —COOH, —CONH₂, —NH—C(O)—O—C₁₋₆ alkyl, C₁₋₆ alkyl, or —C(O)—C₁₋₆ alkyl, wherein the C₁₋₆ alkyl is optionally substituted;
L³ is a bond, or an optionally substituted bivalent C₁₋₈ saturated or unsaturated, straight or branched hydrocarbon chain, wherein 1, 2, or 3 methylene units of the hydrocarbon chain are optionally replaced by —NR—C(O)—, —C(O)—NR—, —C(O)—, —S(O)₂—, —C(O)—O—, —O—C(O)—, —NR—S(O)₂—, or —S(O)₂—NR—;

R⁴ is —NH₂, NHR, —N═C═N—, or —NR₂; and
each R is, independently, H, —OH, —C₁₋₈ alkyl, —OC₁₋₈ alkyl, —C(O)—C₁₋₈ alkyl, —C(O)—OC₁₋₈ alkyl, —O—C(O)—C₁₋₈ alkyl, C₁₋₈ alkyl-O—C(O)—C₁₋₈ alkyl, 4-7 membered monocyclic carbocyclyl, —O-(4-7 membered monocyclic carbocyclyl), —C(O)-(4-7 membered monocyclic carbocyclyl), —C(O)—O-(4-7 membered monocyclic carbocyclyl), phenyl, —O-phenyl, —C(O)-phenyl, —C(O)—O-phenyl, 8-10 membered bicyclic aryl, —O-(8-10 membered bicyclic aryl), —C(O)-(8-10 membered bicyclic aryl), or —C(O)—O-(8-10 membered bicyclic aryl), wherein each of the C₁₋₈ alkyl, 4-7 membered monocyclic carbocyclyl, phenyl, and 8-10 membered bicyclic aryl is optionally and independently substituted.

In some embodiments, compounds having a formula of:

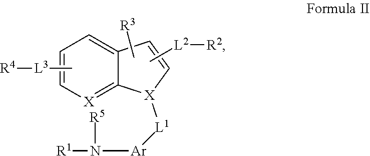

wherein each variable is as defined and provided for herein and, for example, can be selected from the respective groups of chemical moieties described herein.

In some embodiments, provided herein are compounds having a formula of:

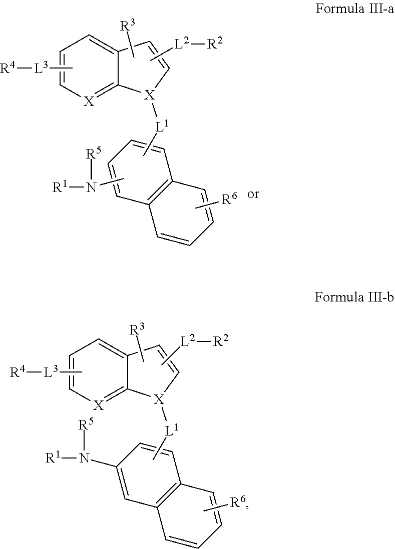

wherein R⁶ is absent, —OH, halogen, —CN, —C(O) H, —NH₂, —NO₂, —COOH, —CONH₂, —CONHR, —NH—C(O)—O—C₁₋₆ alkyl, C₁₋₆ alkyl, or —C(O)—C₁₋₆ alkyl, wherein the C₁₋₆ alkyl is optionally substituted, and the rest variables are as defined and provided for herein.

In some embodiments, provided herein are compounds having a formula of:

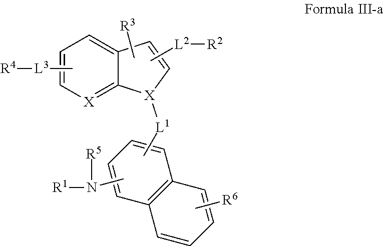

wherein R⁶ is absent, —OH, halogen, —CN, —C(O) H, —NH₂, —NO₂, —COOH, —CONH₂, —CONHR, —NH—C(O)—O—C₁₋₆ alkyl, C₁₋₆ alkyl, or —C(O)—C₁₋₆ alkyl, wherein the C₁₋₆ alkyl is optionally substituted, and the rest variables are as defined and provided for herein.

In some embodiments, provided herein are compounds having a formula of:

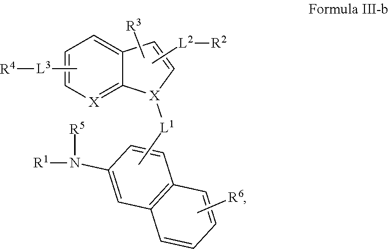

wherein R⁶ is absent, —OH, halogen, —CN, —C(O) H, —NH₂, —NO₂, —COOH, —CONH₂, —CONHR, —NH—C(O)—O—C₁₋₆ alkyl, C₁₋₆ alkyl, or —C(O)—C₁₋₆ alkyl, wherein the C₁₋₆ alkyl is optionally substituted, and the rest variables are as defined and provided for herein.

In some embodiments, the compound has a formula of

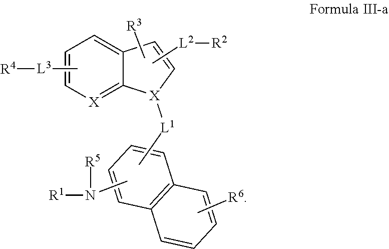

In some embodiments, the compound has a formula of

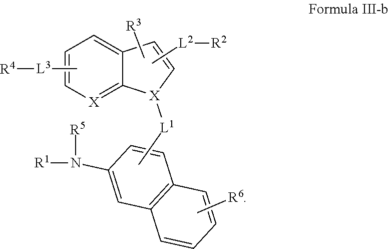

In some embodiments, R⁶ is absent. In some embodiments, R⁶ is —OH. In some embodiments, R⁶ is halogen. In some embodiments, R⁶ is —CN. In some embodiments, R⁶ is —C(O) H. In some embodiments, R⁶ is —NH₂. In some embodiments, R⁶ is —NO₂. In some embodiments, R⁶ is —COOH. In some embodiments, R⁶ is —CONH₂. In some embodiments, R⁶ is —CONHR. In some embodiments, R⁶ is —NH—C(O)—O—C₁₋₆ alkyl. In some embodiments, R⁶ is C₁₋₆ alkyl. In some embodiments, R⁶ is —C(O)—C₁₋₆ alkyl. In some embodiments, the C₁₋₆ alkyl is optionally substituted as defined and provided for herein.

In some embodiments, provided herein are compounds having a formula of:

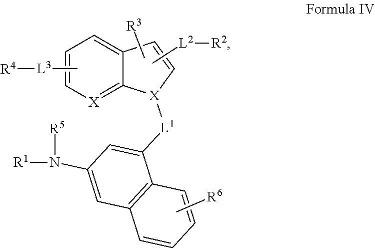

wherein each variable is as provided for herein and, for example, can be selected from the respective groups of chemical moieties described herein.

In some embodiments, provided herein are compounds having a formula of:

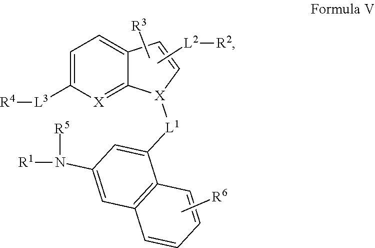

wherein each variable is as provided for herein and, for example, can be selected from the respective groups of chemical moieties described herein.

In some embodiments, provided herein are compounds having a formula of:

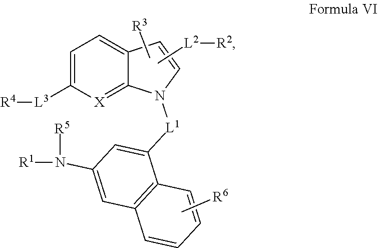

wherein each variable is as provided for herein and, for example, can be selected from the respective groups of chemical moieties described herein.

In some embodiments, provided herein are compounds having a formula of:

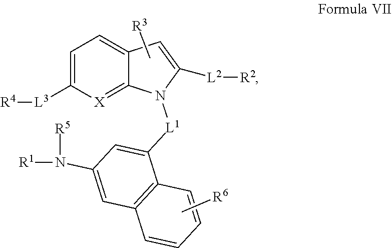

wherein each variable is as provided for herein and, for example, can be selected from the respective groups of chemical moieties described herein.

In some embodiments, the compound of Formula I is of formula:

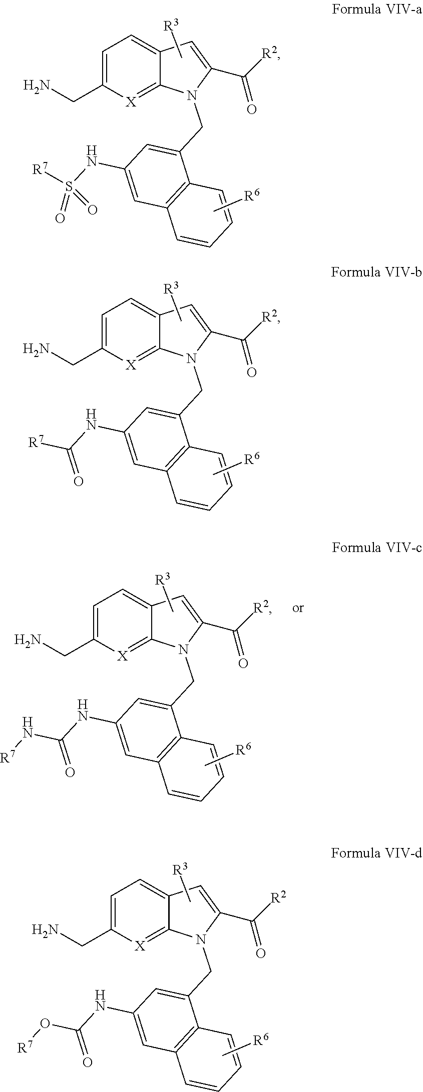

or a pharmaceutically acceptable salt thereof, wherein X, R³, R², R⁶, and R⁷ are as defined herein.

In some embodiments, a compound of Formula I is of formula:

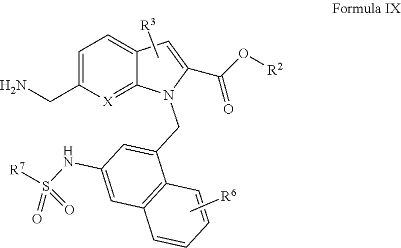

or a pharmaceutically acceptable salt thereof, wherein X, R³, R², R⁶, and R⁷ are as defined herein.

In some embodiments, a compound of Formula I is of formula:

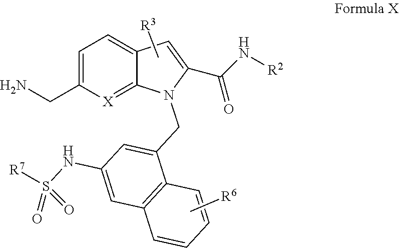

or a pharmaceutically acceptable salt thereof, wherein X, R³, R², R⁶, and R⁷ are as defined herein.

In some embodiments, a compound of Formula I is of the formula:

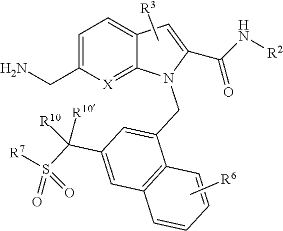

or a pharmaceutically acceptable salt thereof, wherein X, R³, R², R⁶, and R⁷ are as defined herein.

In some embodiments, a compound of Formula I is of the formula:

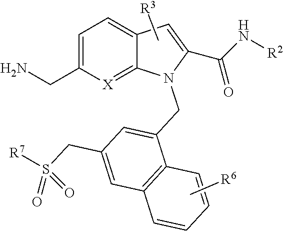

or a pharmaceutically acceptable salt thereof, wherein X, R³, R², R⁶, and R⁷ are as defined herein.

In some embodiments, a compound of Formula I is of the formula:

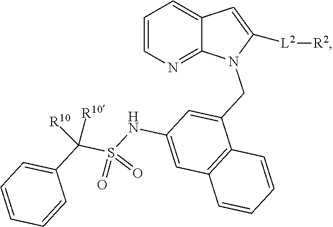

or a pharmaceutically acceptable salt thereof, wherein X, R³, R², R⁶, and R⁷ are as defined herein.

In some embodiments, a compound of Formula I is of the formula:

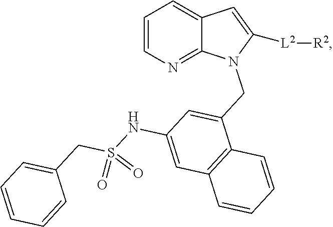

or a pharmaceutically acceptable salt thereof, wherein X, R³, R², R⁶, and R⁷ are as defined herein.

In some embodiments, a compound of Formula I is of the formula:

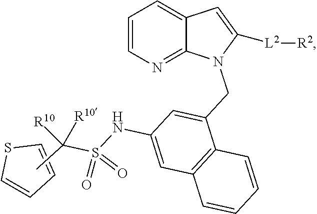

or a pharmaceutically acceptable salt thereof, wherein X, R³, R², R⁶, and R⁷ are as defined herein.

In some embodiments, a compound of Formula I is of the formula:

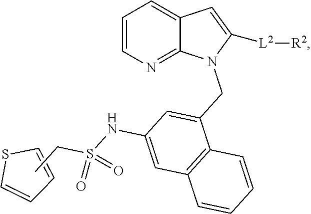

or a pharmaceutically acceptable salt thereof, wherein X, R³, R², R⁶, and R⁷ are as defined herein.

In some embodiments, provided are compounds as described and provided for herein, wherein Ar is an optionally substituted ring selected from phenyl, a 5-6 membered heteroaromatic ring having 1-4 heteroatoms independently selected from nitrogen, oxygen, and sulfur, an 8-10 membered bicyclic aromatic carbocyclic ring, and an 8-10 membered bicyclic heteroaromatic ring having 1-4 heteroatoms independently selected from nitrogen, oxygen, and sulfur. Ar is an optionally substituted ring selected from phenyl. In some embodiments, Ar is a 5-6 membered heteroaromatic ring having 1-4 heteroatoms independently selected from nitrogen. In some embodiments, Ar is oxygen. In some embodiments, Ar is and sulfur. In some embodiments, Ar is an 8-10 membered bicyclic aromatic carbocyclic ring. In some embodiments, Ar is an 8-10 membered bicyclic heteroaromatic ring having 1-4 heteroatoms independently selected from nitrogen, oxygen, and sulfur. In some embodiments, Ar is an 8-10 membered bicyclic aromatic carbocyclic ring or an 8-10 membered bicyclic heteroaromatic ring having 1-4 heteroatoms independently selected from nitrogen, oxygen, and sulfur. In some embodiments, Ar is selected from the group consisting of

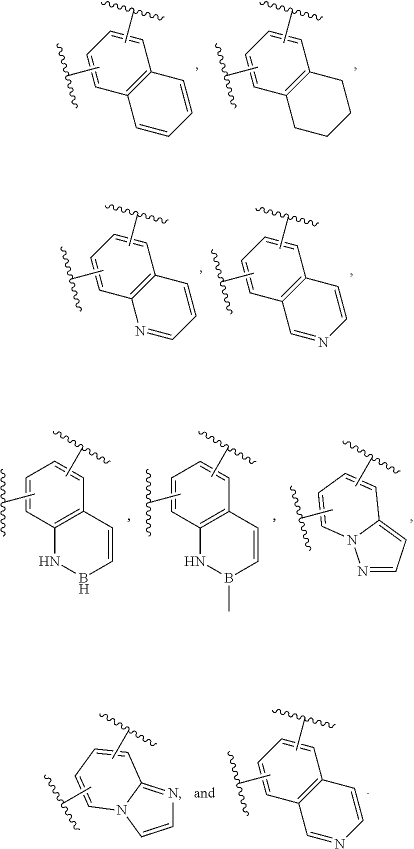

In some embodiments, Ar is selected from the group consisting of

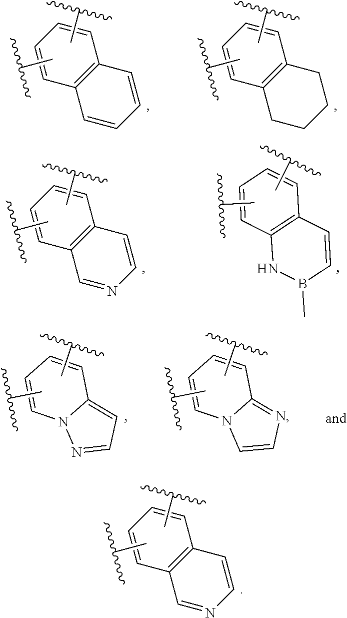

In some embodiments, Ar is

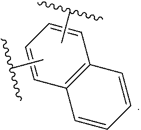

In some embodiments, Ar is

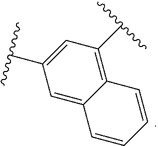 [SMILES: *c1cc(*)c2ccccc2c1]

In some embodiments, Ar is an unsubstituted ring. In certain embodiments, Ar is a substituted ring. In some embodiments. Ar is a substituted ring, substituted with C₁₋₆ alkyl, halo, oxo, —OH, —O—(C₁₋₆ alkyl). In some embodiments, Ar is a substituted ring, wherein the ring is substituted with -Me, —Br, —F, —Cl, ═O, —OH, or —OMe. In some embodiments, Ar is a substituted ring, wherein the ring is substituted with -Me, —OH, or —OMe.

In some embodiments, Ar is selected from those depicted in Table A, Table B, and Table C.

In some embodiments, provided are compounds as described and provided for herein, wherein each X is independently CH, C-L³-R⁴, C-L²-R², C—R³, or N. In some embodiments, provided are compounds as described and provided for herein, wherein each X is independently CH or N. In some embodiments, X is CH. In some embodiments, C-L³-R⁴. In some embodiments, C-L²-R². In some embodiments, C—R³. In some embodiments, X is N. In some embodiments, at least one X is CH. In some embodiments, at least two Xs are CH. In some embodiments, at least three Xs are CH. In some embodiments, at least four Xs are CH. In some embodiments, at least five Xs are CH. In some embodiments, at least six Xs are CH. In some embodiments, one X is CH. In some embodiments, two Xs are CH. In some embodiments, three Xs are CH. In some embodiments, four Xs are CH. In some embodiments, five Xs are CH. In some embodiments, six Xs are CH. In some embodiments, all Xs are CH. In some embodiments, at least one Xs is CH. In some embodiments, at least two Xs are N. In some embodiments, at least three Xs are N. In some embodiments, at least four Xs are N. In some embodiments, at least five Xs are N. In some embodiments, at least six Xs are N. In some embodiments, one Xs is CH. In some embodiments, two Xs are N. In some embodiments, three Xs are N. In some embodiments, four Xs are N. In some embodiments, five Xs are N. In some embodiments, six Xs are N. In some embodiments, all Xs are N. In some embodiments, two instance of X are N. In some embodiments, three instances of X are CH. In some embodiments, one instance of X is C-L³-R⁴. In some embodiments, one instance of X is C-L²-R². In some embodiments, one instance of X is C—R³.

In some embodiments, each X is independently CH or N such that

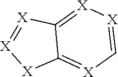

is selected from the following

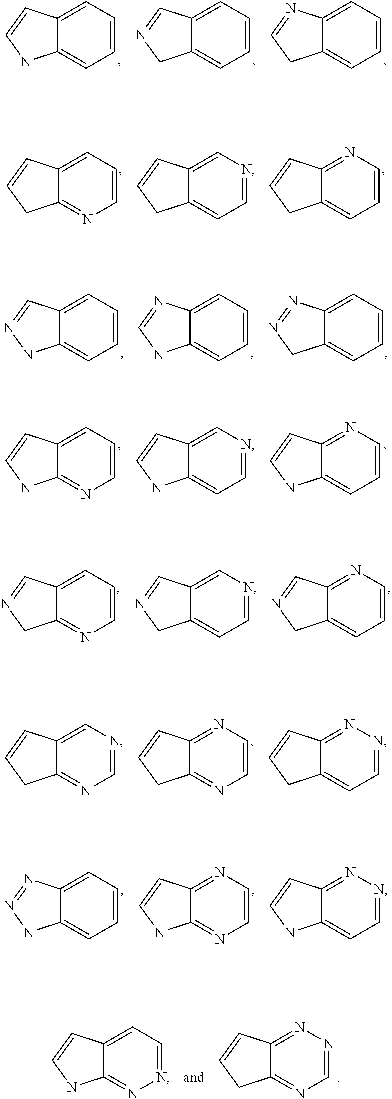

In some embodiments, two instances of X are N and five instances of X are CH. In some embodiments, two instance of X are N, three instances of X are CH, one instance of X is C-L³-R⁴, and one instance of X is C-L²-R². In some embodiments,

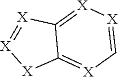

is

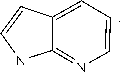

In some embodiments,

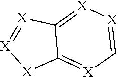

is

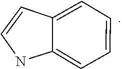

In some embodiments, each X is independently selected from those depicted in Table A, Table B, and Table C.

In some embodiments. Y is a bond, —NR³—, CR¹⁰R^{10′}, or —O—. In some embodiments, provided are compounds as described and provided for herein, wherein Y is —NR⁵—CR¹⁰R^{10′}, or —O—. In some embodiments, Y is —NR⁵—. In some embodiments Y is —NH—. In some embodiments. Y is CR¹⁰R^{10′} In some embodiments. Y is —CH₂—. In some embodiments, Y is —O—. In some embodiments, each Y is selected from those depicted in Table A, Table B, and Table C.

In some embodiments, provided are compounds as described and provided for herein, wherein L¹ is a bond, or an optionally substituted bivalent C₁₋₈ saturated or unsaturated, straight or branched hydrocarbon chain, wherein 1, 2, or 3 methylene units of the hydrocarbon chain are optionally and independently replaced by —S(O)₂—, —C(O)—, —NH—, or —O—. In some embodiments, L¹ is a bond. In some embodiments, L¹ is an optionally substituted C₁₋₈ bivalent hydrocarbon chain, wherein 1 methylene unit of the hydrocarbon chain is optionally replaced by —S(O)₂—, —C(O)—, —NH—, or —O—. In some embodiments, L¹ is an optionally substituted C₁₋₈ bivalent hydrocarbon chain, wherein 2 methylene units of the hydrocarbon chain are optionally and independently replaced by —S(O)₂—, —C(O)—, —NH—, or —O—. In some embodiments, L¹ is an optionally substituted C₁₋₈ bivalent hydrocarbon chain, wherein 3 methylene units of the hydrocarbon chain are optionally and independently replaced by —S(O)₂—, —C(O)—, —NH—, or —O—. In some embodiments, 1 unit of the hydrocarbon chain is optionally and independently replaced by —S(O)₂—, —C(O)—, —NH—, or —O—. In some embodiments, 2 methylene units of the hydrocarbon chain are optionally and independently replaced by —S(O)₂—, —C(O)—, —NH—, or —O—. In some embodiments. 3 methylene units of the hydrocarbon chain are optionally and independently replaced by —S(O)₂—, —C(O)—, —NH—, or —O—. In some embodiments, L¹ is C₁ bivalent hydrocarbon, wherein the 1 methylene unit of the hydrocarbon is optionally replaced by —S(O)₂—, —C(O)—, —NH—, or —O—. In some embodiments, L¹ is C₁ bivalent hydrocarbon, wherein the 1 methylene unit is replaced by —S(O)₂—, —C(O)—, —NH—, or —O—. In some embodiments, L¹ is an optionally substituted bivalent C₁₋₈ saturated or unsaturated, straight or branched hydrocarbon chain, wherein 1, 2, or 3 methylene units of the hydrocarbon chain are optionally and independently replaced by —S(O)₂—, —C(O)—, —NH—. In some embodiments, L¹ is an optionally substituted C₁₋₈ bivalent hydrocarbon chain, wherein 1 methylene unit of the hydrocarbon chain is optionally replaced by —S(O)₂—, —C(O)—, —NH—. In some embodiments, L¹ is an optionally substituted C₁₋₈ bivalent hydrocarbon chain, wherein 2 methylene units of the hydrocarbon chain are optionally and independently replaced by —S(O)₂—, —C(O)—, —NH—. In some embodiments, L¹ is an optionally substituted C₁₋₈ bivalent hydrocarbon chain, wherein 3 methylene units of the hydrocarbon chain are optionally and independently replaced by —S(O)₂—, —C(O)—, —NH—. In some embodiments, 1 unit of the hydrocarbon chain is optionally and independently replaced by —S(O)₂—, —C(O)—, —NH—. In some embodiments, 2 methylene units of the hydrocarbon chain are optionally and independently replaced by —S(O)₂—, —C(O)—, —NH—. In some embodiments. 3 methylene units of the hydrocarbon chain are optionally and independently replaced by —S(O)₂—, —C(O)—, —NH—. In some embodiments, the methylene unit of the hydrocarbon chain is optionally and independently replaced by —S(O)₂—. In some embodiments, the methylene unit of the hydrocarbon chain is optionally and independently replaced by —C(O)—. In some embodiments, the methylene unit of the hydrocarbon chain is optionally and independently replaced by —NH—. In some embodiments, the methylene unit of the hydrocarbon chain is optionally and independently replaced by —O—. In some embodiments, L¹ is —(CH₂)—. In some embodiments, L¹ is —(CH₂)₂—. In some embodiments, L¹ is —(CH₂)₃—. In some embodiments, L¹ is —S(O)₂CH₂—. In some embodiments, L¹ is —S(O)₂(CH₂)₂—. In some embodiments, L¹ is —S(O)₂—. In some embodiments, L¹ is —CH₂S(O)₂—. In some embodiments, L¹ is —(CH₂)₂—S(O)₂—.

In some embodiments, each L¹ is selected from those depicted in Table A, Table B, and Table C.

In some embodiments.

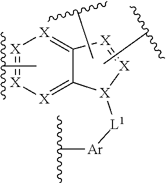

is selected from those depicted in Table A, Table B, and Table C. In some embodiments,

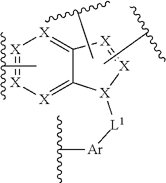

is

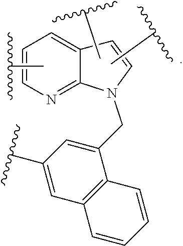

In some embodiments, R¹ is H, D, R⁷, R⁷SO₂—, R⁷S(O)—, R⁷C(O)—, R⁷NR—C(O)—, or R⁷O—C(O)—. In some embodiments, provided are compounds as described and provided for herein, wherein R¹ is H, D, R⁷, R⁷SO₂—, R⁷S(O)—, R⁷C(O)—, or R⁷O—C(O)—. In some embodiments, R¹ is H. In some embodiments, R¹ is D. In some embodiments, R¹ is R⁷. In some embodiments, R¹ is R⁷SO₂—. In some embodiments, R¹ is R⁷S(O)—. In some embodiments, R¹ is R⁷C(O)—. In some embodiments, R¹ is R⁷O—C(O)—. In some embodiments, R¹ is R⁷NR—C(═O)—. In some embodiments, R¹ is R⁷NH—C(═O)—.

In some embodiments, R¹ is selected from those depicted in Table A. Table B, and Table C.

In some embodiments, provided are compounds as described and provided for herein, wherein R⁷ is R¹¹(CR¹⁰R^{10′})ₙ—. In some embodiments, when n is 0, R⁷ is R¹¹. In some embodiments, R⁷ is R¹¹(CR¹⁰R^{10′})—. In some embodiments, R⁷ is R¹¹(CH₂)ₙ—. In some embodiments, R⁷ is R¹¹(CH₂)—. In some embodiments, R⁷ is R¹¹(CHF)—. In some embodiments, R⁷ is R¹¹(CF₂)—. In some embodiments, R⁷ is R¹¹(CH₂)₂—. In some embodiments, R⁷ is R¹¹(CH₂)₃—. In some embodiments, R⁷ is R¹¹(CH₂)₄—. In some embodiments, R⁷ is R¹¹(CH₂)₅—. In some embodiments, R⁷ is as depicted in Table A, Table B, or Table C.

In some embodiments, R¹¹ is H, an optionally substituted C₁₋₆ alkyl, or an optionally substituted ring selected from aryl, a 5-6 membered heteroaromatic ring having 1-4 heteroatoms independently selected from nitrogen, oxygen, and sulfur, a 3-7 membered monocyclic carbocyclic ring, a 4-7 membered heterocyclic ring having 1-3 heteroatoms independently selected from nitrogen, oxygen, and sulfur, a 5-10 membered bicyclic carbocyclic ring, a 7-10 membered bicyclic heterocyclic ring having 1-4 heteroatoms independently selected from nitrogen, oxygen, and sulfur, an 8-10 membered bicyclic aromatic carbocyclic ring, and an 8-10 membered bicyclic heterocyclic ring having 1-3 heteroatoms independently selected from nitrogen, oxygen, and sulfur. As provided herein, R¹¹ is H, an optionally substituted C₁₋₆ alkyl, or an optionally substituted ring selected from phenyl, a 5-6 membered heteroaromatic ring having 1-4 heteroatoms independently selected from nitrogen, oxygen, and sulfur, a 3-7 membered monocyclic carbocyclic ring, a 4-7 membered heterocyclic ring having 1-3 heteroatoms independently selected from nitrogen, oxygen, and sulfur, a 5-10 membered bicyclic carbocyclic ring, a 7-10 membered bicyclic heterocyclic ring having 1-4 heteroatoms independently selected from nitrogen, oxygen, and sulfur, an 8-10 membered bicyclic aromatic carbocyclic ring, and an 8-10 membered bicyclic heterocyclic ring having 1-3 heteroatoms independently selected from nitrogen, oxygen, and sulfur. In some embodiments, provided are compounds as described and provided for herein, wherein R¹¹ is H, an optionally substituted C₁₋₆ alkyl, or an optionally substituted ring selected from phenyl, a 5-7 membered heterocyclic ring having 1-3 heteroatoms independently selected from nitrogen, oxygen, and sulfur, an 8-10 membered bicyclic aromatic carbocyclic ring, and an 8-10 membered bicyclic heterocyclic ring having 1-3 heteroatoms independently selected from nitrogen, oxygen, and sulfur. In some embodiments. R¹¹ is H. In some embodiments, R¹¹ is an optionally substituted C₁₋₆ alkyl. In some embodiments, R¹¹ is a substituted C₁₋₆ alkyl. In certain embodiments, R¹¹ is an unsubstituted C₁₋₆ alkyl. In some embodiments, R¹¹ is an optionally substituted ring selected from phenyl, a 5-7 membered heterocyclic ring having 1-3 heteroatoms independently selected from nitrogen, oxygen, and sulfur, an 8-10 membered bicyclic aromatic carbocyclic ring, and an 8-10 membered bicyclic heterocyclic ring having 1-3 heteroatoms independently selected from nitrogen, oxygen, and sulfur. In some embodiments, R¹¹ is a substituted ring. In certain embodiments R¹¹ is an substituted ring. In some embodiments, R¹¹ is an optionally substituted aryl. In some embodiments, R¹¹ is an optionally substituted phenyl. In some embodiments, R¹¹ is an optionally substituted 5-6 membered heteroaromatic ring having 1-4 heteroatoms independently selected from nitrogen, oxygen, and sulfur. In some embodiments, R¹¹ is a optionally substituted 3-7 membered monocyclic carbocyclic ring. In some embodiments, R¹ is an optionally substituted 5-7 membered heterocyclic ring having 1-3 heteroatoms independently selected from nitrogen, oxygen, and sulfur. In some embodiments, R¹¹ is an optionally substituted 4-7 membered heterocyclic ring having 1-3 heteroatoms independently selected from nitrogen, oxygen, and sulfur. In some embodiments, R¹¹ is an optionally substituted 5-10 membered bicyclic carbocyclic ring. In some embodiments, R¹¹ is an optionally substituted 7-10 membered bicyclic heterocyclic ring having 1-4 heteroatoms independently selected from nitrogen, oxygen, and sulfur. In some embodiments, R¹¹ is an optionally substituted 8-10 membered bicyclic aromatic carbocyclic ring. In some embodiments, R¹¹ is an optionally substituted 8-10 membered bicyclic heterocyclic ring having 1-3 heteroatoms independently selected from nitrogen, oxygen, and sulfur. In some embodiments, R¹¹ is an optionally substituted 8-10 membered bicyclic heterocyclic ring having 1-3 heteroatoms independently selected from nitrogen, oxygen, and sulfur. In some embodiments, R¹¹ is phenyl or thiophenyl (thienyl). In some embodiments, R¹¹ is phenyl. In some embodiments, R¹¹ is thiophenyl (thienyl).

In some embodiments, when R¹¹ is substituted C₁₋₆ alkyl or a substituted ring, the one or more substituents are independently selected from halo, C₁₋₆ alkyl, C₁₋₆ haloalkyl, —OH, —O—(C₁₋₆ alkyl), —NR₂, —CN, oxo, —NO₂, —(C₁₋₆ alkyl)-O—(C₁₋₆ alkyl), —(C₁₋₆ alkyl)-O)—(C₁₋₆ alkyl)-O—(C₁₋₆ alkyl), —NRC(═O)OR, —C(═O)R—C(═O)OR, —OC(═O)R, —C(═O)NR₂, —NHC(═O)R, C₂₋₆ alkene, phenyl, —S(—O)R. and —S(═O)₂R. In some embodiments, when R¹¹ is substituted C₁₋₆ alkyl or a substituted ring, the one or more substituents are independently selected from —F, —Cl, —Br, —CH₃, —CH₂CH₃, —CH(CH₃)₂, —OH, —OMe, —NH₂, —NH(CH₃), —N(CH₃)₂, —N(CH₂CH₃)₂, —N(CH₂CH₂CH₃)₂, —CN, —NO₂, —CF₃, ═O, —CH₂OCH₃, —CH₂OCH₂OCH₃, —OC(═O)Me, —C(═O)OH, —C(═O)OMe, —C(═O)OtBu, —C(═O)NH₂—C(═O)NHCH₃, —C(═O)N(CH₃)₂), —NHC(═O)Me, —NHC(═O)OtBu, —CH═CH₂, phenyl, —S(═O)₂Me, —S(═O)₂tBu. and —S(═O)₂(cyclopropyl).

In some embodiments, provided are compounds as described and provided for herein, wherein R¹¹ is

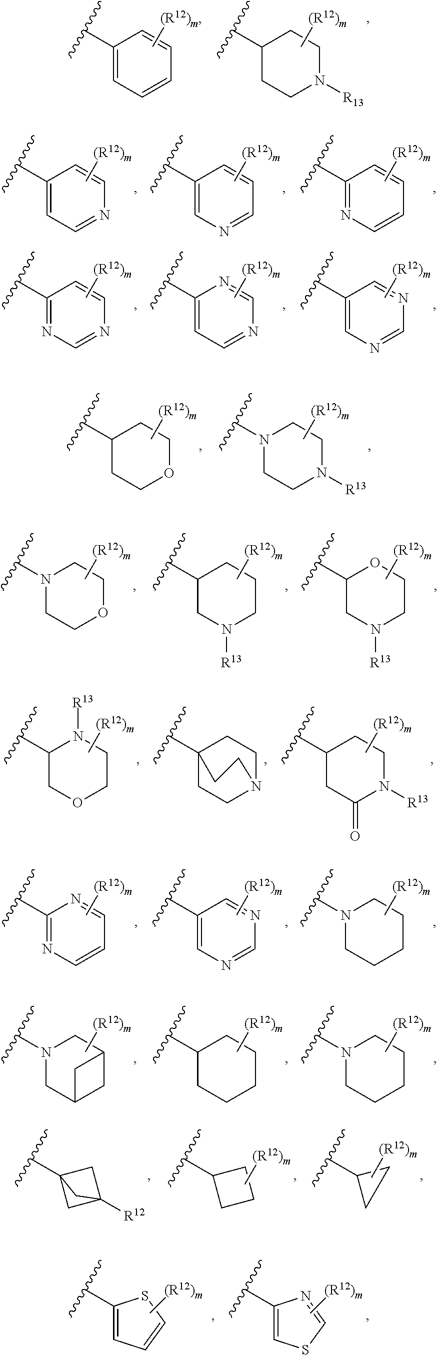

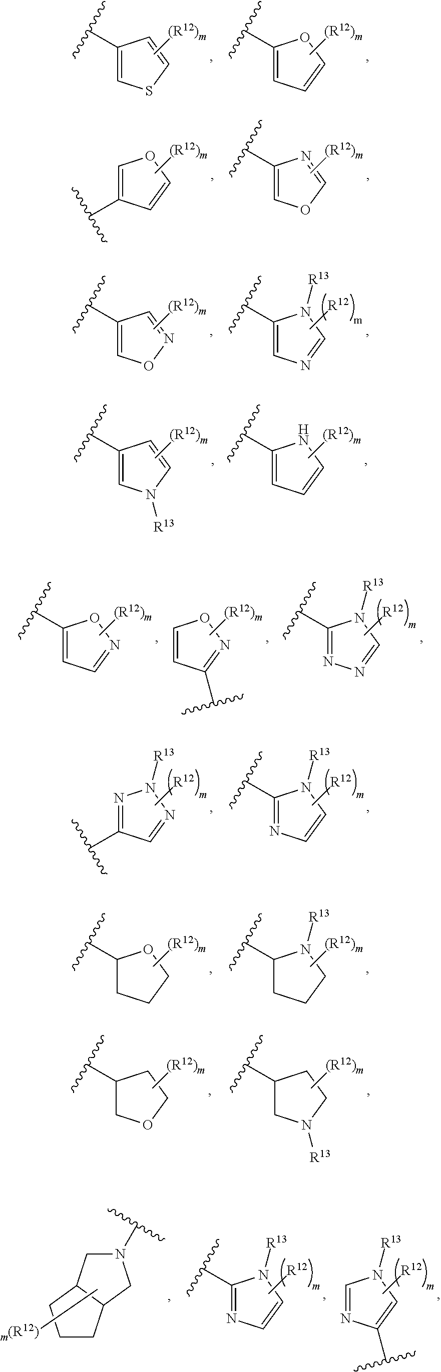

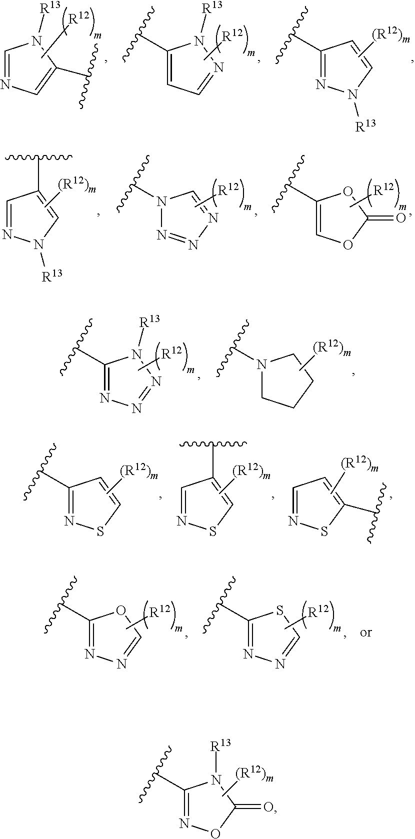

wherein m is 0-5 and R¹² and R¹³ are each, independently, hydrogen, —OH, halogen, —CN, —C(O) H, —NH₂, —NHR, —NR₂—NO₂, —COOH, —CONH₂, —NH—C(O)—O—C₁₋₆ alkyl, C₁₋₆ alkyl, —(O)—C₁₋₆ alkyl, haloalkyl, ═O, —(C₁₋₆ alkyl)-O—(C₁₋₆ alkyl), (C₁₋₆ alkyl)-O—(C₁₋₆ alkyl)-O—(C₁₋₆ alkyl), —C(O)NH—C₁₋₆ alkyl, —C(O)—C₁₋₆ alkyl, —O—C(O)—C₁₋₆ alkyl, —C(O)—OC₁₋₆ alkyl, —CH—CH₂, phenyl, —S(═O)₂ (C₁₋₆ alkyl), or —S(═O)2(carbocyclic) wherein the C₁₋₆ alkyl is optionally substituted. In some embodiments, provided are compounds as described and provided for herein, wherein R¹¹ is

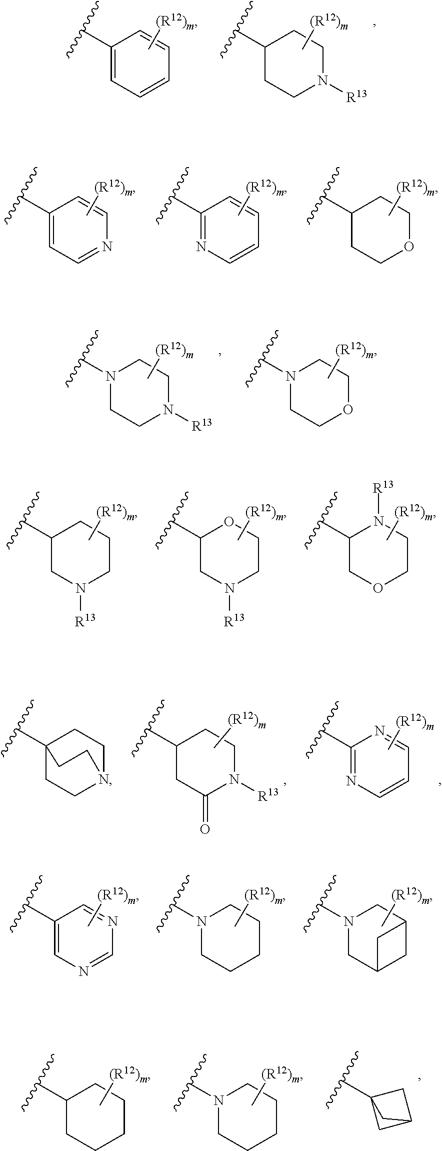

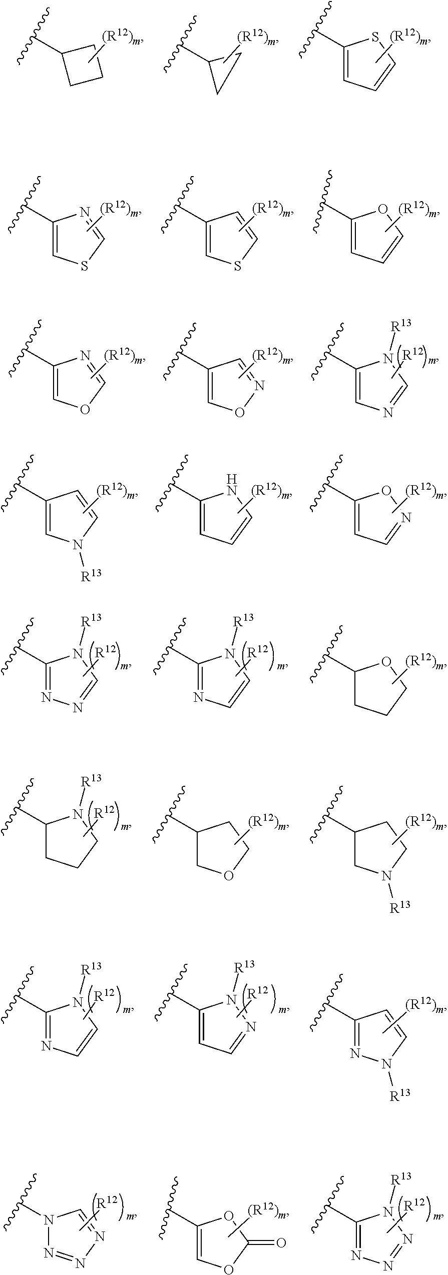

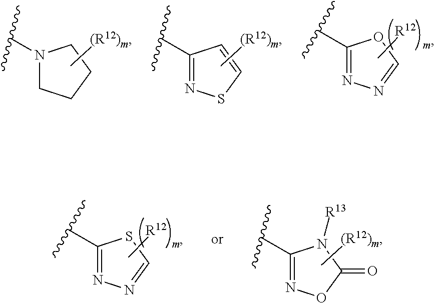

wherein m is 0-5 and R¹² and R¹³ are each, independently, H, —OH, halogen, —CN, —C(O) H, —NH₂, —NHR, —NR₂—NO₂, —COOH, —CONH₂, —NH—C(O)—O—C₁₋₆ alkyl, C₁₋₆ alkyl, —C(O)NH—C₁₋₆ alkyl, —C(O)—C₁₋₆ alkyl, —O—C(O)—C₁₋₆ alkyl, or —C(O)—OC₁₋₆ alkyl, wherein the C₁₋₆ alkyl is optionally substituted. In some embodiments, R¹¹ is

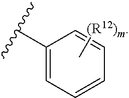

In some embodiments, R¹¹ is

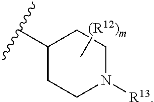

In some embodiments, R¹¹ is

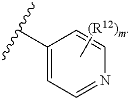

In some embodiments, R¹¹ is

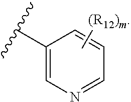

In some embodiments, R¹¹ is

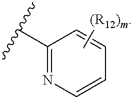

In some embodiments, R¹¹ is

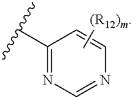

In some embodiments, R¹¹ is

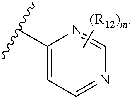

In some embodiments, R¹¹ is

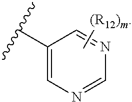

In some embodiments, R¹¹ is

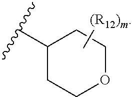

In some embodiments, R¹¹ is

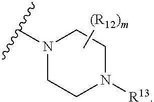

In some embodiments, R¹¹ is

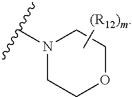

In some embodiments, R¹¹ is

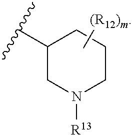

In some embodiments, R¹¹ is

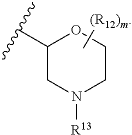

In some embodiments, R¹¹ is

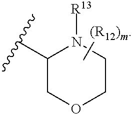

In some embodiments, R¹¹ is

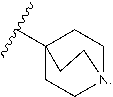 [SMILES: *C12CCN(CC1)CC2]

In some embodiments, R¹¹ is

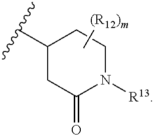

In some embodiments, R¹¹ is

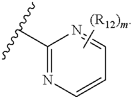

In some embodiments, R¹¹ is

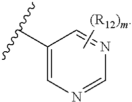

In some embodiments, R¹¹ is

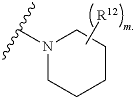

In some embodiments, R¹¹ is

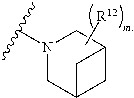

In some embodiments, R¹¹ is

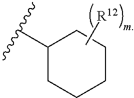

In some embodiments, R¹¹ is

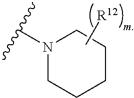

In some embodiments, R¹¹ is

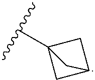 [SMILES: *C12CC(C1)C2]

In some embodiments, R¹¹ is

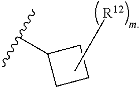

In some embodiments, R¹¹ is

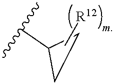

In some embodiments, R¹¹ is

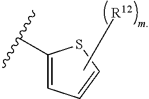

In some embodiments, R¹¹ is

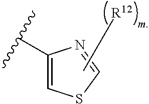

In some embodiments, R¹¹ is

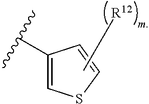

In some embodiments, R¹¹ is

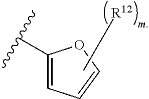

In some embodiments, R¹¹ is

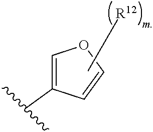

In some embodiments, R¹¹ is

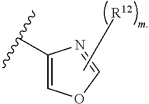

In some embodiments, R¹¹ is

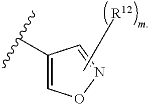

In some embodiments, R¹¹ is

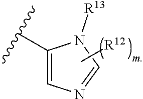

In some embodiments, R¹¹ is

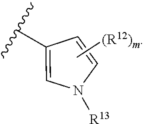

In some embodiments, R¹¹ is

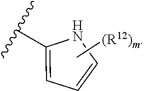

In some embodiments, R¹¹ is

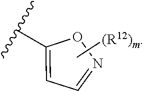

In some embodiments, R¹¹ is

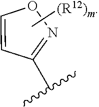

In some embodiments, R¹¹ is

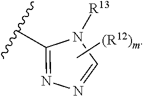

In some embodiments, R¹¹ is

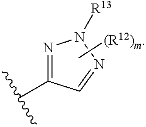

In some embodiments, R¹¹ is

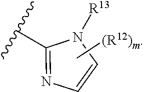

In some embodiments, R¹¹ is

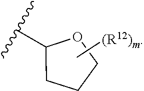

In some embodiments, R¹¹ is

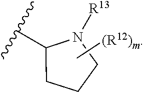

In some embodiments, R¹¹ is

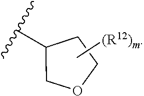

In some embodiments, R¹¹ is

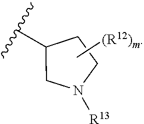

In some embodiments, R¹¹ is

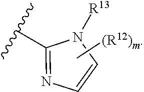

In some embodiments, R¹¹ is

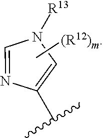

In some embodiments, R¹¹ is

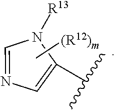

In some embodiments, R¹¹ is

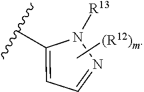

In some embodiments, R¹¹ is

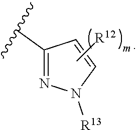

In some embodiments, R¹¹ is

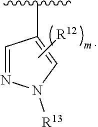

In some embodiments, R¹¹ is

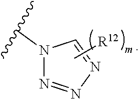

In some embodiments, R¹¹ is

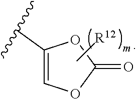

In some embodiments, R¹¹ is

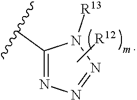

In some embodiments, R¹¹ is

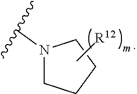

In some embodiments, R¹¹ is

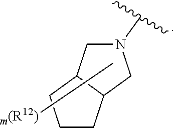

In some embodiments, R¹¹ is

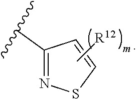

In some embodiments, R¹¹ is

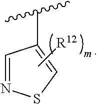

In some embodiments, R¹¹ is

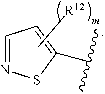

In some embodiments, R¹¹ is

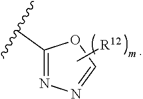

In some embodiments, R¹¹ is

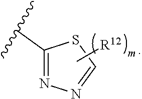

In some embodiments, R¹¹ is

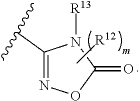

In some embodiments, wherein m is 0-5 and R¹² and R¹³ are each, independently, hydrogen, —OH, halogen, —CN, —C(O) H, —NH₂, —NHR, —NR₂—NO₂, —COOH, —CONH₂, —NH—C(O)—O—C₁₋₆ alkyl, C₁₋₆ alkyl, —(O)—C₁₋₆ alkyl, haloalkyl, ═O, —(C₁₋₆ alkyl)-O—(C₁₋₆ alkyl), —(C₁₋₆ alkyl)-O—(C₁₋₆ alkyl)-O—(C₁₋₆ alkyl), —C(O)NH—C₁₋₆ alkyl, —C(O)—C₁₋₆ alkyl, —O—C(O)—C₁₋₆ alkyl, —C(O)—OC₁₋₆ alkyl, —CH═CH₂, phenyl, —S(═O)₂ (C₁₋₆ alkyl), or —S(═O)₂ (carbocyclic), wherein C₁₋₆ alkyl is optionally substituted. In some embodiments, wherein m is 0-5 and R¹² and R¹³ are each, independently, —OH, halogen, —CN, —C(O) H, —NH₂, —NHR, —NR₂—NO₂, —COOH, —CONH₂, —NH—C(O)—O—C₁₋₆ alkyl, C₁₋₆ alkyl, —C(O)NH—C₁₋₆ alkyl, —C(O)—C₁₋₆ alkyl, —O—C(O)—C₁₋₆ alkyl, or —C(O)—OC₁₋₆ alkyl, wherein C₁₋₆ alkyl is optionally substituted. In some embodiments, m is 0-5. In some embodiments, m is 0-4. In some embodiments, m is 0-3. In some embodiments, m is 0-2. In some embodiments, m is 0-1. In some embodiments, m is 1-5. In some embodiments, m is 1-4. In some embodiments, m is 1-3. In some embodiments, m is 1-2. In some embodiments, m is 2-5. In some embodiments, m is 2-4. In some embodiments, m is 2-3. In some embodiments, m is 3-5. In some embodiments, m is 3-4. In some embodiments, m is 4-5. In some embodiments, m is 0. In some embodiments, m is 1. In some embodiments, m is 2. In some embodiments, m is 3. In some embodiments, m is 4. In some embodiments, m is 5. In some embodiments, R¹² and R¹³ are each, independently hydrogen, halo, C₁₋₆ alkyl, C₁₋₆ haloalkyl, —OH, —O—(C₁₋₆ alkyl), —NR₂, —CN, oxo, —NO₂, —(C₁₋₆ alkyl)-O—(C₁₋₆ alkyl), —(C₁₋₆ alkyl)-O—(C₁₋₆ alkyl)-O)—(C₁₋₆ alkyl), —NRC(═O)OR, —C(═O)R—C(═O)OR, —OC(═O)R, —C(═O)NR₂, —NHC(═O)R, C₂₋₆ alkene, phenyl, —S(═O)R, or —S(═O)₂R. In some embodiments, R¹² and R¹³ are each, independently hydrogen, —F, —Cl, —Br. —CH₃, —CH—CH₃, —CH(CH₃)₂, —OH, —OMe, —NH₂, —NH(CH₃), —N(CH₃)₂, —N(CH₂CH₃)₂, —N(CH₂CH₂CH₃)₂, —CN, —NO₂, —CF₃, ═O, —CH₂OCH₃, —CH₂OCH₂OCH₃, —OC(═O)Me, —C(═O)OH, —C(═O)OMe, —C(═O)OtBu, —C(═O)NH₂, —C(═O)NHCH₃, —C(═O)N(CH₃)₂, —NHC(═O)Me, —NHC(═O)OtBu, —CH═CH₂, phenyl, —S(═O)₂Me, —S(═O)₂tBu, or —S(═O)₂(cyclopropyl). In some embodiments, R¹² is —OH, halogen, —CN, —C(O) H, —NH₂, —NO₂, —COOH, —CONH₂, —NH—C(O)—O—C₁₋₆ alkyl, C₁₋₆ alkyl, —C(O)NH—C₁₋₆ alkyl, —C(O)—C₁₋₆ alkyl, —O—C(O)—C₁₋₆ alkyl, or —C(O)—OC₁₋₆ alkyl, wherein the C₁₋₆ alkyl is optionally substituted. In some embodiments, R¹² is absent. In some embodiments, R¹² is H. In some embodiments, R¹² is —OH. In some embodiments, R¹² is a halogen. In some embodiments, R¹² is —F. In some embodiments, R¹² is bromo. In some embodiments, R¹² is chloro. In some embodiments, R¹² is fluoro. In some embodiments, R¹² is —CN. In some embodiments, R¹² is —C(O) H. In some embodiments, R¹² is —NR₂. In some embodiments, R¹² is —NH₂. In some embodiments, R¹² is —NH(CH₃). In some embodiments, R¹² is —N(CH₃)₂. In some embodiments, R¹² is —N(CH—CH₃)₂. In some embodiments, R¹² is —N(CH₂CH—CH₃)₂. In some embodiments, R¹² is ═O). In some embodiments, R¹² is —NO₂. In some embodiments, R¹² is —CN. In some embodiments, R¹² is —COOH. In some embodiments, R¹² is —CONH₂. In some embodiments, R¹² is —NR—C(O)—OR. In some embodiments, R¹² is —NH—C(O)—O—C₁₋₆ alkyl. In some embodiments, R¹² is —NHC(═O)OMe. In some embodiments, R¹² is —NHC(═O)OtBu. In some embodiments, R¹² is C₁₋₆ alkyl. In some embodiments, R¹² is methyl. In some embodiments, R¹² is ethyl. In some embodiments, R¹² is isopropyl. In some embodiments. R¹² is C₁₋₆ haloalkyl. In some embodiments, R¹² is CF₃. In some embodiments, R¹² is —O—(C₁₋₆ alkyl). In some embodiments, R¹² is —OMe. In some embodiments, R¹² is —OEt. In some embodiments, R¹² is —(C₁₋₆ alkyl)-O—(C₁₋₆ alkyl). In some embodiments, R¹² is —CH₂OCH₃. In some embodiments, R¹² is —(C₁₋₆ alkyl)-O—(C₁₋₆ alkyl)-O—(C₁₋₆ alkyl). In some embodiments, R¹² is CH₂OCH₂OCH₃. In some embodiments, R¹² is —C(O)NR₂. In some embodiments, R¹² is —C(═O)N(CH₃)₂. In some embodiments, R¹² is —C(O)NH—C₁₋₆ alkyl. In some embodiments, R¹² is —C(═O)NHCH₃. In some embodiments, R¹² is —NHC(—O)R. In some embodiments R¹² is —NHC(═O)Me. In some embodiments, R¹² is —COR. In some embodiments, R¹² is —COH. In some embodiments, R¹² is —C(O)—C₁₋₆ alkyl. In some embodiments, R¹² is —C(═O) CH₃. In some embodiments, R¹² is —O—C(O)R. In some embodiments, R¹² is —O—C(O)—C₁₋₆ alkyl. In some embodiments, R¹² is —OC(═O)Me. In some embodiments, R¹² is —C(O)—OR. In some embodiments, R¹² is —C(O)—OC₁₋₆ alkyl. In some embodiments, R¹² is —C(═O)OMe. In some embodiments, R¹² is —C(═O)OtBu. In some embodiments, R¹² is C₂₋₆ alkene. In some embodiments, R¹² is —CH—CH₂. In some embodiments, R¹² is phenyl. In some embodiments, R¹² is —S(═O)R. In some embodiments, R¹² is —S(═O)₂R. In some embodiments, R¹² is S(═O)₂Me. In some embodiments, R¹² is —S(═O)₂tBu. In some embodiments, R¹² is S(═O)₂(cyclopropyl).

In some embodiments, R¹³ is hydrogen, —OH, halogen, —CN, —C(O) H, —NH₂, —NO₂, —COOH, —CONH₂, —NH—C(O)—O—C₁₋₆ alkyl, C₁₋₆ alkyl, —C(O)NH—C₁₋₆ alkyl, —C(O)—C₁₋₆ alkyl, —O—C(O)—C₁₋₆ alkyl, or —C(O)—OC₁₋₆ alkyl, wherein the C₁₋₆ alkyl is optionally substituted. In some embodiments, R¹³ is —OH, halogen, —CN, —C(O) H, —NH₂—NO₂, —COOH, —CONH₂, —NH—C(O)—O—C₁₋₆ alkyl, C₁₋₆ alkyl, —C(O)NH—C₁₋₆ alkyl, —C(O)—C₁₋₆ alkyl, —O—C(O)—C₁₋₆ alkyl, or —C(O)—OC₁₋₆ alkyl, wherein the C₁₋₆ alkyl is optionally substituted. In some embodiments, R¹³ is absent. In some embodiments, R¹³ is H. In some embodiments, R¹³ is hydrogen, optionally substituted C₁₋₆ alkyl, or halogen. In some embodiments, R¹³ is C₁₋₆ alkyl. In some embodiments, R¹³ is methyl. In some embodiments, R¹³ is ethyl. In some embodiments, R¹³ is hydrogen, —OH, fluoro. In some embodiments, R¹³ is —OH. In some embodiments, R¹³ is halogen. In some embodiments, R¹³ is —CN. In some embodiments, R¹³ is —C(O) H. In some embodiments, R¹³ is —NH₂. In some embodiments, R¹³ is —NO₂. In some embodiments, R¹³ is —COOH. In some embodiments, R¹³ is —CONH₂. In some embodiments, R¹³ is —NH—C(O)—O—C₁₋₆ alkyl. In some embodiments, R¹³ is C₁₋₆ alkyl. In some embodiments, R¹³ is —C(O)NH—C₁₋₆ alkyl. In some embodiments, R¹³ is —C(O)—C₁₋₆ alkyl. In some embodiments, R¹³ is —O—C(O)—C₁₋₆ alkyl. In some embodiments, R¹³ is —C(O)—OC₁₋₆ alkyl. In some embodiments, the C₁₋₆ alkyl is optionally substituted as described and provided herein.

In some embodiments, R¹¹ is as depicted in Table A. Table B, or Table C.

In some embodiments, provided are compounds as described and provided for herein, wherein R⁵, R¹⁰, and R^{10′} are each, independently, H, D, halo, oxo, C₁₋₆ alkyl, C₂₋₆ alkenyl, C₂₋₆ alkynyl, C₁₋₆ haloalkyl, C₃₋₆ cycloalkyl, CN, NO₂, C(O)NR₂, C(O)OR, S(O)OR, SO₂OR, S(O)NR₂, SO₂NR₂, or B(OR)₂.

In some embodiments, provided are compounds as described and provided for herein, wherein R⁵ is H, D, halo, oxo, C₁₋₆ alkyl, C₂₋₆ alkenyl, C₂₋₆ alkynyl, C₁₋₆ haloalkyl, C₃₋₆ cycloalkyl, CN, NO₂, C(O)NR₂, C(O)OR, S(O)OR, SO₂OR, S(O)NR₂, SO₂NR₂, or B(OR)₂. In some embodiments. R⁵ is H. In some embodiments. R⁵ is D. In some embodiments. R⁵ is halo. In some embodiments, R⁵ is oxo. In some embodiments. R⁵ is C₁₋₆ alkyl. In some embodiments, R⁵ is C₂₋₆ alkenyl. In some embodiments, R⁵ is C₂₋₆ alkynyl. In some embodiments, R⁵ is C₁₋₆ haloalkyl. In some embodiments. R⁵ is C₃₋₆ cycloalkyl. In some embodiments, R⁵ is CN. In some embodiments, R⁵ is NO₂. In some embodiments, R⁵ is C(O)NR₂. In some embodiments, R⁵ is C(O)OR. In some embodiments, R⁵ is B(OR)₂.

In some embodiments, provided are compounds as described and provided for herein, wherein R¹⁰ is H, D, halo, oxo, C₁₋₆ alkyl, C₂₋₆ alkenyl, C₂₋₆ alkynyl, C₁₋₆ haloalkyl. C₃₋₆ cycloalkyl, CN. NO₂, C(O)NR₂, C(O)OR, S(O)OR, SO₂OR, S(O)NR₂, SO₂NR₂, or B(OR)₂. In some embodiments, R¹⁰ is H. In some embodiments, R¹⁰ is D. In some embodiments, R¹⁰ is halo. In some embodiments, R¹⁰ is oxo. In some embodiments, R¹⁰ is C₁₋₆ alkyl. In some embodiments, R¹⁰ is C₂₋₆ alkenyl. In some embodiments, R¹⁰ is C₂₋₆ alkynyl. In some embodiments, R¹⁰ is C₁₋₆ haloalkyl. In some embodiments, R¹⁰ is C₃₋₆ cycloalkyl. In some embodiments, R¹⁰ is CN. In some embodiments, R¹⁰ is NO₂. In some embodiments, R¹⁰ is C(O)NR₂. In some embodiments, R¹⁰ is C(O)OR. In some embodiments. R¹⁰ is B(OR)₂.

In some embodiments, provided are compounds as described and provided for herein, wherein R^{10′} is H, D, halo, oxo, C₁₋₆ alkyl, C₂₋₆ alkenyl, C₂₋₆ alkynyl, C₁₋₆ haloalkyl, C₃₋₆ cycloalkyl, CN, NO₂, C(O)NR₂, C(O)OR, S(O)OR, SO₂OR, S(O)NR₂, SO₂NR₂, or B(OR)₂. In some embodiments, R^{10′} is H. In some embodiments, R^{10′} is D. In some embodiments, R^{10′} is halo. In some embodiments, R^{10′} is oxo. In some embodiments, R^{10′} is C₁₋₆ alkyl. In some embodiments, R^{10′} is C₂₋₆ alkenyl. In some embodiments, R^{10′} is C₂₋₆ alkynyl. In some embodiments, R^{10′} is C₁₋₆ haloalkyl. In some embodiments, R^{10′} is C₃₋₆ cycloalkyl. In some embodiments, R^{10′} is CN. In some embodiments, R^{10′} is NO₂. In some embodiments, R^{10′} is C(O)NR₂. In some embodiments, R^{10′} is C(O)OR. In some embodiments, R^{10′} is B(OR)₂.

In some embodiments, R¹⁰ and R^{10′} together with the carbon atom to which they are both attached form an optionally substituted C₃₋₇ spirocyclic ring.

In some embodiments, provided are compounds as described and provided for herein, wherein n is 0-8. In some embodiments, n is 0-7. In some embodiments, n is 0-6. In some embodiments, n is 0-5. In some embodiments, n is 0-4. In some embodiments, n is 0-3. In some embodiments, n is 0-2. In some embodiments, n is 0-1. In some embodiments, n is 1-8. In some embodiments, n is 1-7. In some embodiments, n is 1-6. In some embodiments, n is 1-5. In some embodiments, n is 1-4. In some embodiments, n is 1-3. In some embodiments, n is 1-2. In some embodiments, n is 2-8. In some embodiments, n is 2-7. In some embodiments, n is 2-6. In some embodiments, n is 2-5. In some embodiments, n is 2-4. In some embodiments, n is 2-3. In some embodiments, n is 3-8. In some embodiments, n is 3-7. In some embodiments, n is 3-6. In some embodiments, n is 3-5. In some embodiments, n is 3-4. In some embodiments, n is 4-8. In some embodiments, n is 4-7. In some embodiments, n is 4-6. In some embodiments, n is 4-5. In some embodiments, n is 5-8. In some embodiments, n is 5-7. In some embodiments, n is 5-6. In some embodiments, n is 6-8. In some embodiments, n is 6-7. In some embodiments, n is 7-8. In some embodiments, n is 0. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8.

In some embodiments, provided are compounds as described and provided for herein, wherein L² is an optionally substituted bivalent C₁₋₃ saturated or unsaturated, straight or branched hydrocarbon chain, wherein 1, 2, or 3 methylene units of the hydrocarbon chain are optionally and independently replaced by —NR—C(O)—, —C(O)—NR—, —C(O)—, —S(O)₂—, —C(O)—O—, —O—C(O)—, —NR—S(O)₂—, —S(O)₂—NR—, or -Cy-. In some embodiments, L² is a substituted bivalent C₁₋₈ saturated or unsaturated, straight or branched hydrocarbon chain, wherein 1, 2, or 3 methylene units of the hydrocarbon chain are optionally and independently replaced by —NR—C(O)—, —C(O)—NR—, —C(O)—, —S(O)₂—, —C(O)—O—, —O—C(O)—, —NR—S(O)₂—, —S(O))₂—NR—, or -Cy-. In some embodiments, L² is an unsubstituted bivalent C₁₋₈ saturated or unsaturated, straight or branched hydrocarbon chain, wherein 1, 2, or 3 methylene units of the hydrocarbon chain are optionally and independently replaced by —NR—C(O)—, —C(O)—NR—, —C(O)—, —S(O)₂—, —C(O)—O—, —O—C(O)—, —NR—S(O)₂—, —S(O)₂—NR—, or -Cy-. In some embodiments, L² is an optionally substituted bivalent C₁₋₈ saturated or unsaturated hydrocarbon chain. In some embodiments, L² is an optionally substituted bivalent C₁₋₈ unsaturated hydrocarbon chain. In some embodiments, L² is an optionally substituted bivalent C₁₋₈ unsaturated hydrocarbon chain. In some embodiments, L² is a straight or branched hydrocarbon chain. In some embodiments, L² is a straight hydrocarbon chain. In some embodiments, L² is a branched hydrocarbon chain. In some embodiments, 1, 2, or 3 methylene units of the hydrocarbon chain are optionally and independently replaced by —NR—C(O))—, —C(O)—NR—, —C(O)—, —S(O)₂—, —C(O)—O—, —O—C(O)—, —NR—S(O)₂—, —S(O)₂—NR—, or -Cy-. In some embodiments, 1 methylene unit of the hydrocarbon chain is optionally and independently replaced by —NR—C(O)—, —C(O)—NR—, —C(O)—, —S(O)₂—, —C(O)—O—, —O—C(O)—, —NR—S(O)₂—, —S(O)₂—NR—, or -Cy-. In some embodiments, 2 methylene units of the hydrocarbon chain are optionally and independently replaced by —NR—C(O)—, —C(O)—NR—, —C(O)—, —S(O)₂—, —C(O)—O—, —O—C(O)—, —NR—S(O)₂—, —S(O)₂—NR—, or -Cy-. In some embodiments. 3 methylene units of the hydrocarbon chain are optionally and independently replaced by —NR—C(O)—, —C(O)—NR—, —C(O)—, —S(O)₂—, —C(O)—O—, —O—C(O)—, —NR—S(O)₂—, —S(O)₂—NR—, or -Cy-. In some embodiments, L² is C₁ hydrocarbon, wherein the 1 methylene unit optionally is independently replaced by —NR—C(O)—, —C(O)—NR—, —C(O)—, —S(O)₂—, —C(O)—O—, —O—C(O)—, —NR—S(O)₂—, —S(O)₂—NR—, or -Cy-. In some embodiments, L² is C₁ hydrocarbon chain wherein the 1 methylene unit of the chain is independently replaced by —NR—C(O)—, —C(O)—NR—, —C(O)—, —S(O)₂—, —C(O)—O—, —O—C(O)—, —NR—S(O)₂—, —S(O)₂—NR—, or -Cy-. In some embodiments, one or more methylene units of the hydrocarbon chain are optionally and independently replaced by —NR—C(O)—. In some embodiments, one or more methylene units of the hydrocarbon chain is optionally and independently replaced by —C(O)—NR—. In some embodiments, one or more methylene units of the hydrocarbon chain are optionally and independently replaced by —C(O)—. In some embodiments, one or more methylene units of the hydrocarbon chain are optionally and independently replaced by —S(O)₂—. In some embodiments, one or more methylene units of the hydrocarbon chain are optionally and independently replaced by —C(O)—O—. In some embodiments, one or more methylene units of the hydrocarbon chain are optionally and independently replaced by —O—C(O)—. In some embodiments, one or more methylene units of the hydrocarbon chain are optionally and independently replaced by —NR—S(O)₂—. In some embodiments, one or more methylene units of the hydrocarbon chain are optionally and independently replaced by —S(O)₂—NR—. In some embodiments, one or more methylene units of the hydrocarbon chain are optionally and independently replaced by -Cy-.

In some embodiments, L² is as depicted in Table A, Table B, or Table C.

In some embodiments, -Cy- is an optionally substituted bivalent ring selected from phenyl, a 4-6 membered monocyclic heterocyclic ring having 1-4 heteroatoms independently selected from nitrogen, oxygen, and sulfur, a 5-10 membered bicyclic carbocyclic ring, and a 4-6 membered monocyclic heteroaromatic ring having 1-4 heteroatoms independently selected from nitrogen, oxygen, and sulfur. In some embodiments. -Cy- is an optionally substituted bivalent ring selected from phenyl, a 4-6 membered monocyclic heterocyclic ring having 1-4 heteroatoms independently selected from nitrogen, oxygen, and sulfur, and a 4-6 membered monocyclic heteroaromatic ring having 1-4 heteroatoms independently selected from nitrogen, oxygen, and sulfur. In some embodiments, -Cy- is an optionally substituted phenyl. In some embodiments, -Cy- is an optionally substituted 4-6 membered monocyclic heterocyclic ring having 1-4 heteroatoms independently selected from nitrogen, oxygen, and sulfur. In some embodiments, -Cy- is an optionally substituted 4-6 membered monocyclic heteroaromatic ring having 1-4 heteroatoms independently selected from nitrogen, oxygen, and sulfur. In some embodiments, -Cy- is

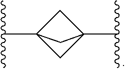 [SMILES: *C12CC(*)(C1)C2]

In some embodiments, L² is —CH₂—, —CH₂CH₂—, —NH—, —NH—CH₃—, —CH₃—NH—, —NH—C(O)—, —N(CH₃)—C(O)—, —S(O)₂—, —CH₂—NH—C(O)—,

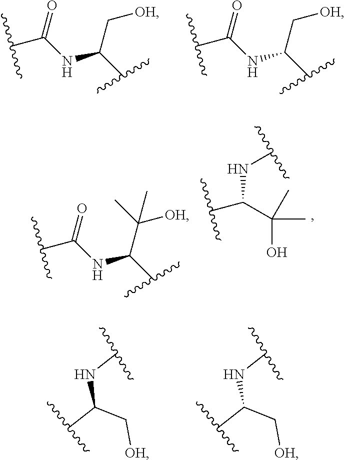

(CH₂)₂—, —NH—CH(CH₃)—, —NH—(CH₂)₂—,

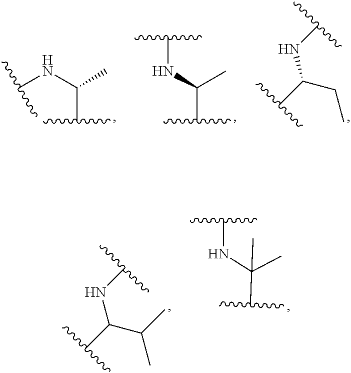

—NH—(CH₂)₄—NH—, or —NH—(CH₂)₃—. In some embodiments, L² is —CH₂—. In some embodiments, L² is —CH₂CH₂—. In some embodiments, L² is —NH—. In some embodiments, L² is —NH—CH₃—. In some embodiments, L² is —CH₃—NH—. In some embodiments, L² is —NH—C(O)—. In some embodiments, L² is —N(CH₃)—C(O)—. In some embodiments, L² is —S(O)₂—. In some embodiments, L² is —CH₂—NH—C(O)—. In some embodiments, L² is

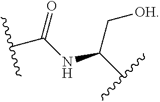 [SMILES: *C(=O)N[C@H](*)CO]

In some embodiments, L² is

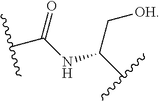 [SMILES: *C(=O)N[C@@H](*)CO]

In some embodiments, L² is

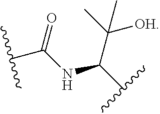 [SMILES: *C(=O)N[C@H](*)C(C)(C)O]

In some embodiments, L² is

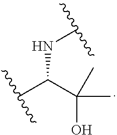 [SMILES: *N[C@@H](*)C(C)(C)O]

In some embodiments, L² is

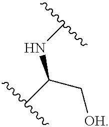 [SMILES: *N[C@H](*)CO]

In some embodiments, L² is

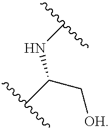 [SMILES: *N[C@@H](*)CO]

In some embodiments, L² is —(CH₂)₂—. In some embodiments, L² is —NH—CH(CH₃)—. In some embodiments, L² is —NH—(CH₂)₂—. In some embodiments, L² is

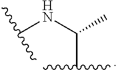 [SMILES: *N[C@@H](*)C]

In some embodiments, L² is

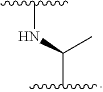 [SMILES: *N[C@H](*)C]

In some embodiments, L² is

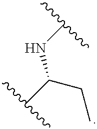 [SMILES: *N[C@@H](*)CC]

In some embodiments, L² is

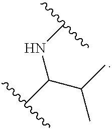 [SMILES: *NC(*)C(C)C]

In some embodiments, L² is

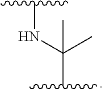 [SMILES: *NC(*)(C)C]

In some embodiments, L² is —NH—(CH₂)₄—NH—. In some embodiments, L² is —NH—(CH₂)₃—. In some embodiments, L² is —C(O)— or —CH₂CH₂—.

In some embodiments, L² is selected from those depicted in Table A, Table B, or Table C.

In some embodiments, provided are compounds as described and provided for herein, wherein R² is R¹⁴ (CR¹⁵R^{15′})ₚ—. In some embodiments, when p is 0, R² is R¹⁴. In some embodiments, R² is R¹⁴ (CH₂)ₚ—. In some embodiments, R² is R¹⁴ (CR¹⁵R^{15′})—. In some embodiments, R² is R¹⁴ (CH₂)—. In some embodiments, R² is R¹⁴ (CH₂)₂—. In some embodiments, R² is R¹⁴ (CH₂)₃—. In some embodiments, R² is R¹⁴ (CH₂)₄—. In some embodiments, R² is R¹⁴ (CH₂)₅—.

In some embodiments, R² is as depicted in Table A, Table B, or Table C.

In some embodiments, R¹⁴ is H, D, —OH, —NR₂, —O(C₁₋₆ alkyl), —O(C₁₋₆ alkyl)- (optionally substituted 4-7 membered monocyclic heterocyclic ring), an optionally substituted C₁₋₆ alkyl, or an optionally substituted ring selected from phenyl, a 3-7 membered monocyclic carbocyclic ring, a 4-7 membered monocyclic heterocyclic ring having 1-4 heteroatoms independently selected from nitrogen, oxygen, and sulfur, a 5-10 membered bicyclic carbocyclic ring, a 7-10 membered bicyclic heterocyclic ring having 1-4 heteroatoms independently selected from nitrogen, oxygen, and sulfur, phenyl, 5-6 membered monocyclic heteroaromatic ring having 1-4 heteroatoms independently selected from nitrogen, oxygen, and sulfur, a 8-10 membered bicyclic aromatic ring, a 8-10 membered bicyclic heteroaromatic ring having 1-4 heteroatoms independently selected from nitrogen, oxygen, and sulfur, and adamantyl. In some embodiments, R¹⁴ is H, D, an optionally substituted C₁₋₆ alkyl, or an optionally substituted ring selected from a 4-7 membered monocyclic carbocyclic ring, a 4-7 membered monocyclic heterocyclic ring having 1-4 heteroatoms independently selected from nitrogen, oxygen, and sulfur, a 7-10 membered bicyclic carbocyclic ring, a 7-10 membered bicyclic heterocyclic ring having 1-4 heteroatoms independently selected from nitrogen, oxygen, and sulfur, phenyl, 5-6 membered monocyclic heteroaromatic ring having 1-4 heteroatoms independently selected from nitrogen, oxygen, and sulfur, a 8-10 membered bicyclic aromatic ring, a 8-10 membered bicyclic heteroaromatic ring having 1-4 heteroatoms independently selected from nitrogen, oxygen, and sulfur, and adamantyl. In some embodiments, R¹⁴ is H. In some embodiments, R¹⁴ is D. In some embodiments, R¹⁴ is —OH. In some embodiments, R¹⁴ is —NR₂. In some embodiments, R¹⁴ is —NH₂. In some embodiments, R¹⁴ is —NH(C₁₋₆ alkyl). In some embodiments, R¹⁴ is —NHCH₃. In some embodiments, R¹⁴ is —N(CH₃)₂. In some embodiments, R¹⁴ is —N(C₁₋₆ alkyl) z. In some embodiments, R¹⁴ is —O—(C₁₋₆ alkyl). In some embodiments, R¹⁴ is —OMe. In some embodiments, R¹⁴ is —OEt. In some embodiments, R¹⁴ is —OtBu. In some embodiments, R¹⁴ is —O(C₁₋₆ alkyl)-(optionally substituted 4-7 membered monocyclic heterocyclic ring). In some embodiments, R¹⁴ is an optionally substituted C₁₋₆ alkyl. In some embodiments, R¹⁴ is an optionally substituted ring selected from a 3-7 membered monocyclic carbocyclic ring, a 4-7 membered monocyclic heterocyclic ring having 1-4 heteroatoms independently selected from nitrogen, oxygen, and sulfur, a 5-10 membered bicyclic carbocyclic ring, a 7-10 membered bicyclic heterocyclic ring having 1-4 heteroatoms independently selected from nitrogen, oxygen, and sulfur, phenyl, 5-6 membered monocyclic heteroaromatic ring having 1-4 heteroatoms independently selected from nitrogen, oxygen, and sulfur, a 8-10 membered bicyclic aromatic ring, a 8-10 membered bicyclic heteroaromatic ring having 1-4 heteroatoms independently selected from nitrogen, oxygen, and sulfur, and adamantyl. In some embodiments, R¹⁴ is an optionally substituted ring selected from a 4-7 membered monocyclic carbocyclic ring, a 4-7 membered monocyclic heterocyclic ring having 1-4 heteroatoms independently selected from nitrogen, oxygen, and sulfur, a 7-10 membered bicyclic carbocyclic ring, a 7-10 membered bicyclic heterocyclic ring having 1-4 heteroatoms independently selected from nitrogen, oxygen, and sulfur, phenyl, 5-6 membered monocyclic heteroaromatic ring having 1-4 heteroatoms independently selected from nitrogen, oxygen, and sulfur, a 8-10 membered bicyclic aromatic ring, a 8-10 membered bicyclic heteroaromatic ring having 1-4 heteroatoms independently selected from nitrogen, oxygen, and sulfur, and adamantyl. In some embodiments, R¹⁴ is an optionally substituted 4-7 membered monocyclic carbocyclic ring. In some embodiments, R¹⁴ is an optionally substituted 4-7 membered monocyclic heterocyclic ring having 1-4 heteroatoms independently selected from nitrogen, oxygen, and sulfur. In some embodiments, R¹⁴ is an optionally substituted 7-10 membered bicyclic carbocyclic ring. In some embodiments, R¹⁴ is an optionally substituted 7-10 membered bicyclic heterocyclic ring having 1-4 heteroatoms independently selected from nitrogen, oxygen, and sulfur, or phenyl. In some embodiments, R¹⁴ is an optionally substituted 7-10 membered bicyclic heterocyclic ring having 1-4 heteroatoms independently selected from nitrogen, oxygen, and sulfur. In some embodiments, R¹⁴ is an optionally substituted phenyl. In some embodiments, R¹⁴ is an optionally substituted 5-6 membered monocyclic heteroaromatic ring having 1-4 heteroatoms independently selected from nitrogen, oxygen, and sulfur. In some embodiments, R¹⁴ is an optionally substituted 8-10 membered bicyclic aromatic ring, an 8-10 membered bicyclic heteroaromatic ring having 1-4 heteroatoms independently selected from nitrogen, oxygen, and sulfur, or adamantyl. In some embodiments, R¹⁴ is an optionally substituted 8-10 membered bicyclic aromatic ring or an 8-10 membered bicyclic heteroaromatic ring having 1-4 heteroatoms independently selected from nitrogen, oxygen, and sulfur. In some embodiments, R¹⁴ is an optionally substituted adamantyl.

In some embodiments, when R¹⁴ is optionally substituted C₁₋₆ alkyl or an optionally substituted ring, the one or more substituents on the C₁₋₆ alkyl or ring are independently selected from halo, C₁₋₆ alkyl, C₁₋₆ haloalkyl, —OH, —O—(C₁₋₆ alkyl), —NR₂, —CN, oxo, —NO₂, —(C₁₋₆ alkyl)-O—(C₁₋₆ alkyl), —(C₁₋₆ alkyl)-O—(C₁₋₆ alkyl)-O—(C₁₋₆ alkyl), —NRC(═O)OR, —C(═O)R—C(═O)OR, —OC(═O)R, —C(═O)NR₂, —NHC(═O)R, C₂₋₆ alkene, phenyl, —S(═O)R, —S(═O)₂R, and a 3-7 membered monocyclic carbocyclic ring. In some embodiments, when R¹⁴ is optionally substituted C₁₋₆ alkyl or an optionally substituted ring, the one or more substituents are independently selected from —F, —Cl, —Br, —CH₃, —CH₂CH₃, —CH(CH₃)₂, —C(CH₃)₃, —OH, —OMe, —NH₂, —NH(CH₃), —N(CH₃)₂, —N(CH₂CH₃)₂, —N(CH₂CH₂CH₃)₂, —CN, —NO₂, —CF₃, ═O, —CH₂OCH₃, —CH₂OCH₂OCH₃, —OC(═O)Me, —C(═O)OH, —C(═O)OMe, —C(═O)OtBu, —C(═O)NH₂, —C(═O)NHCH₃, —C(═O)N(CH₃)₂, —NHC(═O)Me, —NHC(═O)OtBu, —CH═CH₂, phenyl, —S(═O)₂Me, —S(═O)₂tBu, —S(═O)₂(cyclopropyl), and cyclopropyl. In some embodiments, when R¹⁴ is optionally substituted C₁₋₆ alkyl or an optionally substituted ring, the optional substituents on the C₁₋₆ alkyl or ring are independently selected from C₁₋₆ alkyl, —NR₂, —CN, ═O, and a 3-7 membered monocyclic carbocyclic ring. In some embodiments, when R¹⁴ is optionally substituted C₁₋₆ alkyl or an optionally substituted ring, the optional substituents are independently selected from the C₁₋₆ alkyl or ring is methyl, ethyl, isopropyl, tert-butyl-NH₂, —NHMe, —NMe₂, —CN, ═O, and cyclopropyl.

In some embodiments, provided are compounds as described and provided for herein, wherein R¹⁴ is

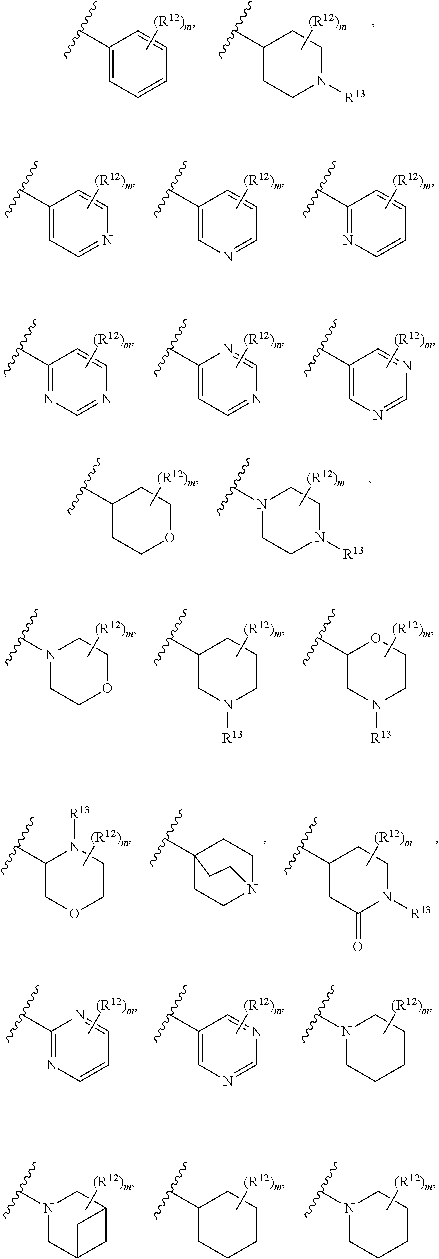

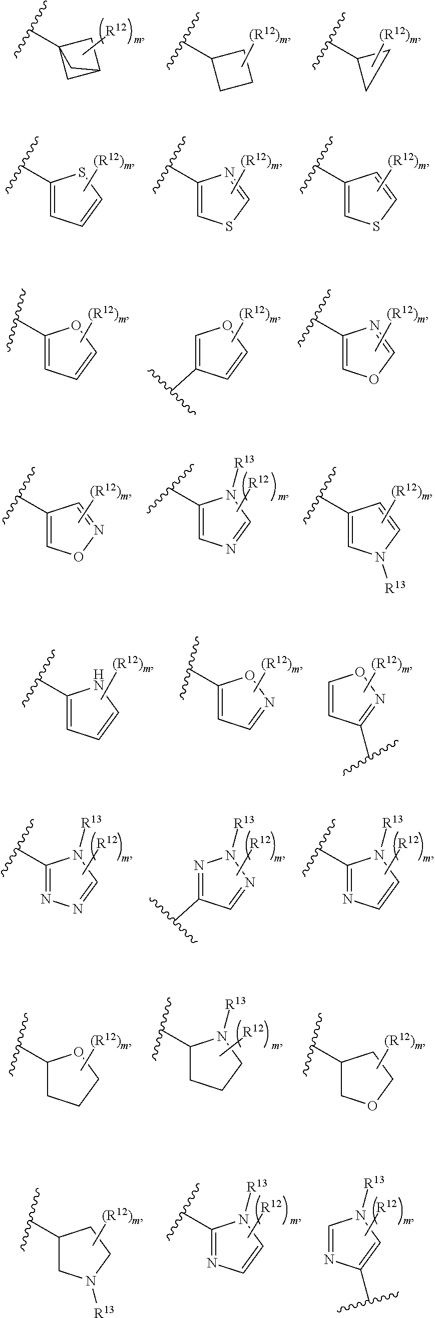

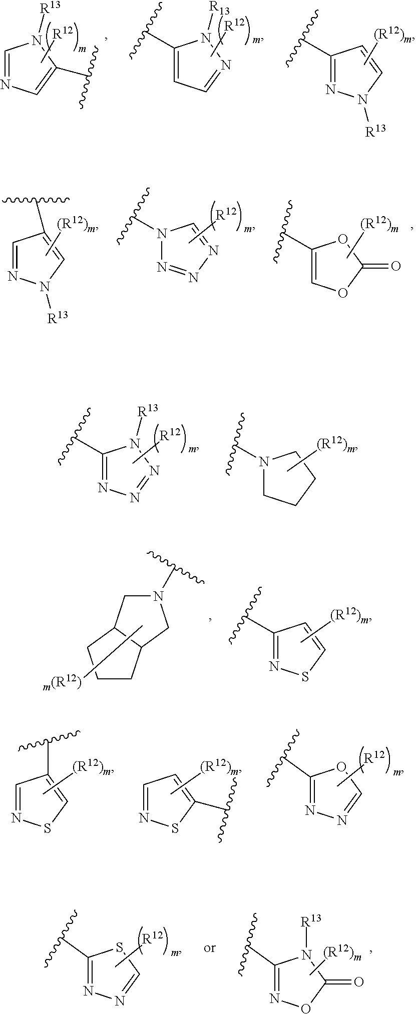

wherein m is 0-5 and R¹² and R¹³ are each, independently, hydrogen, —OH, halogen, —CN, —C(O) H, —NH₂, —NHR, —NR₂—NO₂, —COOH, —CONH₂, —NH—C(O)—O—C₁₋₆ alkyl, C₁₋₆ alkyl, —(O)—C₁₋₆ alkyl, haloalkyl, ═O, —(C₁₋₆ alkyl)-O—(C₁₋₆ alkyl), —(C₁₋₆ alkyl)-O—(C₁₋₆ alkyl)-O—(C₁₋₆ alkyl), —C(O)NH—C₁₋₆ alkyl, —C(O)—C₁₋₆ alkyl, —O—C(O)—C₁₋₆ alkyl, —C(O)—OC₁₋₆ alkyl, —CH═CH₂, phenyl, —S(═O)₂ (C₁₋₆ alkyl), —S(═O). (carbocyclic ring), or a 3-7 membered monocyclic carbocyclic ring, wherein the C₁₋₆ alkyl is optionally substituted. In some embodiments, provided are compounds as described and provided for herein, wherein R¹⁴ is

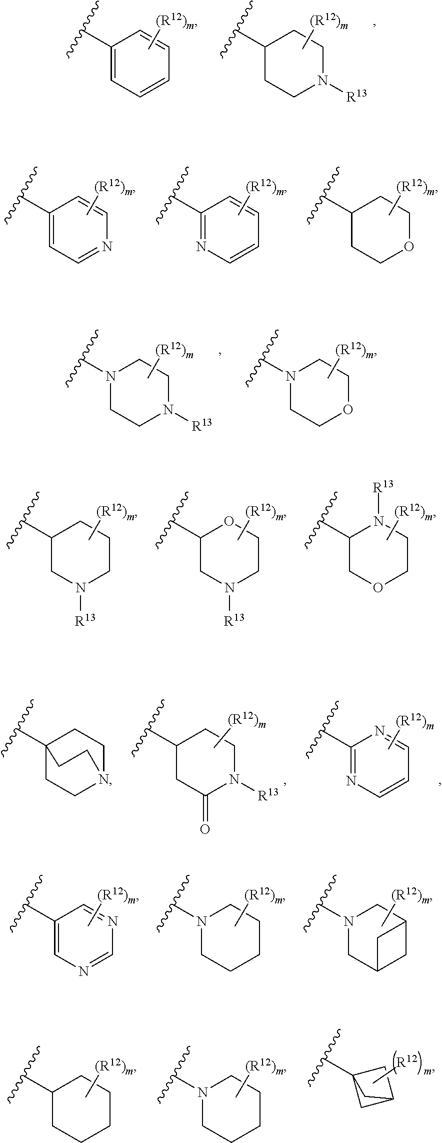

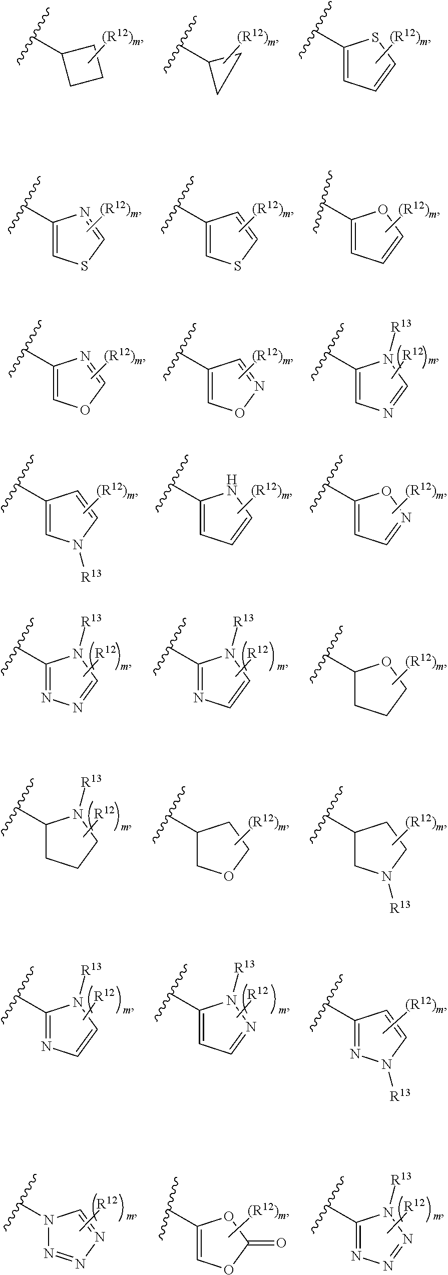

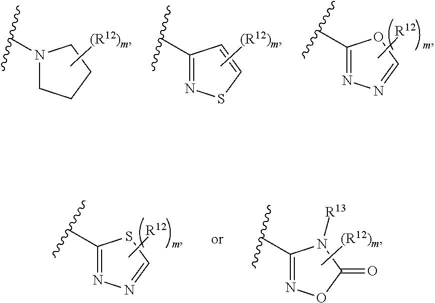

wherein m is 0-5 and R¹² and R¹³ are each, independently, —OH, halogen, —CN, —C(O) H, —NH₂, —NHR, —NR₂, —NO₂, —COOH, —CONH₂, —NH—C(O)—O—C₁₋₆ alkyl, C₁₋₆ alkyl, —C(O)NH—C₁₋₆ alkyl, —C(O)—C₁₋₆ alkyl, —O—C(O)—C₁₋₆ alkyl, or —C(O)—OC₁₋₆ alkyl, wherein the C₁₋₆ alkyl is optionally substituted. In some embodiments, R¹⁴ is

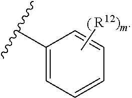

In some embodiments, R¹⁴ is

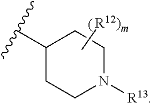

In some embodiments, R¹⁴ is

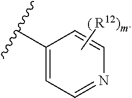

In some embodiments, R¹⁴ is

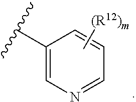

In some embodiments, R¹⁴ is

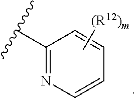

In some embodiments, R¹⁴ is

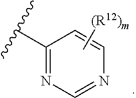

In some embodiments, R¹⁴ is

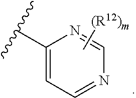

In some embodiments, R¹⁴ is

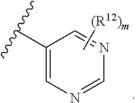

In some embodiments, R¹⁴ is

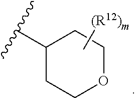

In some embodiments, R¹⁴ is

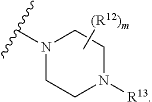

In some embodiments, R¹⁴ is

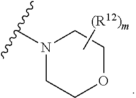

In some embodiments, R¹⁴ is

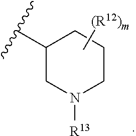

In some embodiments, R¹⁴ is

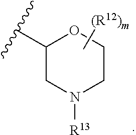

In some embodiments, R¹⁴ is

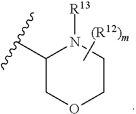

In some embodiments, R¹⁴ is

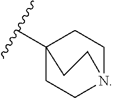 [SMILES: *C12CCN(CC1)CC2]

In some embodiments, R¹⁴ is

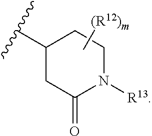

In some embodiments, R¹⁴ is

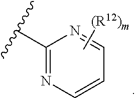

In some embodiments, R¹⁴ is

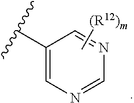

In some embodiments, R¹⁴ is

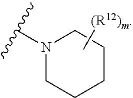

In some embodiments, R¹⁴ is

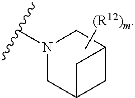

In some embodiments, R¹⁴ is

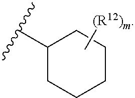

In some embodiments, R¹⁴ is

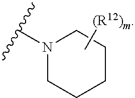

In some embodiments, R¹⁴ is

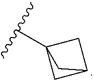 [SMILES: *C12CC(C1)C2]

In some embodiments, R¹⁴ is

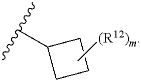

In some embodiments, R¹⁴ is

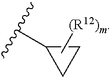

In some embodiments, R¹⁴ is

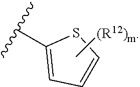

In some embodiments, R¹⁴ is

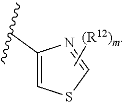

In some embodiments, R¹⁴ is

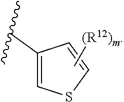

In some embodiments, R¹⁴ is

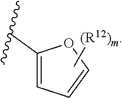

In some embodiments, R¹⁴ is

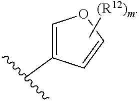

In some embodiments, R¹⁴ is

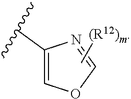

In some embodiments, R¹⁴ is

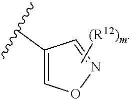

In some embodiments, R¹⁴ is

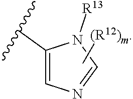

In some embodiments, R¹⁴ is

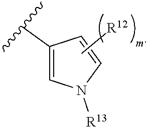

In some embodiments, R¹⁴ is

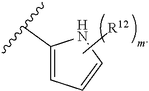

In some embodiments, R¹⁴ is

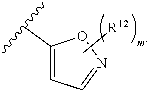

In some embodiments, R¹⁴ is

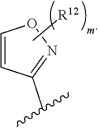

In some embodiments, R¹⁴ is

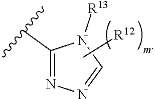

In some embodiments, R¹⁴ is

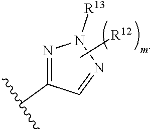

In some embodiments, R¹⁴ is

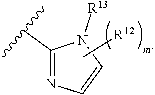

In some embodiments, R¹⁴ is

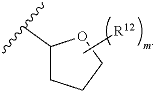

In some embodiments, R¹⁴ is

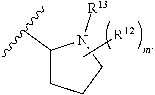

In some embodiments, R¹⁴ is

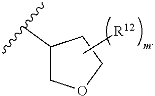

In some embodiments, R¹⁴ is

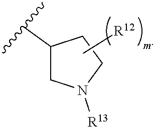

In some embodiments, R¹⁴ is

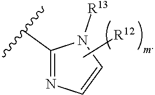

In some embodiments, R¹⁴ is

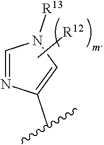

In some embodiments, R¹⁴ is

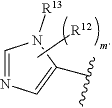

In some embodiments, R¹⁴ is

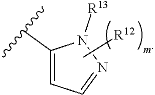

In some embodiments, R¹⁴ is

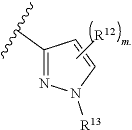

In some embodiments, R¹⁴ is

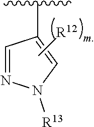

In some embodiments, R¹⁴ is

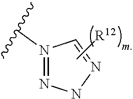

In some embodiments, R¹⁴ is

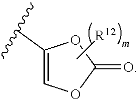

In some embodiments, R¹⁴ is

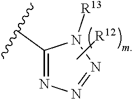

In some embodiments, R¹⁴ is

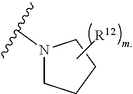

In some embodiments, R¹⁴ is

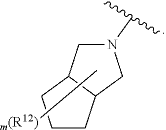

In some embodiments, R¹⁴ is

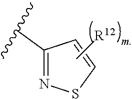

In some embodiments, R¹⁴ is

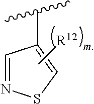

In some embodiments, R¹⁴ is

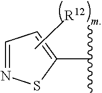

In some embodiments, R¹⁴ is

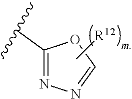

In some embodiments, R¹⁴ is

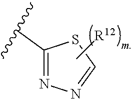

In some embodiments, R¹⁴ is

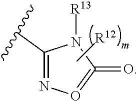

In some embodiments, R¹⁴ is

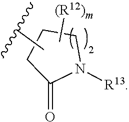

In some embodiments, R¹⁴ is as depicted in Table A, Table B, or Table C.

In some embodiments, wherein m is 0-5 and R¹² and R¹³ are each, independently, hydrogen, —OH, halogen, —CN, —C(O) H, —NH₂, —NHR, —NR₂, —NO₂, —COOH, —CONH₂, —NH—C(O)—O—C₁₋₆ alkyl, C₁₋₆ alkyl, —(O)—C₁₋₆ alkyl, haloalkyl, ═O, —(C₁₋₆ alkyl)-O—(C₁₋₆ alkyl), —(C₁₋₆ alkyl)-O—(C₁₋₆ alkyl)-O—(C₁₋₆ alkyl), —C(O)NH—C₁₋₆ alkyl, —C(O)—C₁₋₆ alkyl, —O—C(O)—C₁₋₆ alkyl, —C(O)—OC₁₋₆ alkyl, —CH═CH₂, phenyl, —S(═O)₂ (C₁₋₆ alkyl), —S(═O)₂ (carbocyclic), or a 3-7 membered monocyclic carbocyclic ring, wherein the C₁₋₆ alkyl is optionally substituted. In some embodiments, wherein m is 0-5 and R¹² and R¹³ are each, independently, —OH, halogen, —CN, —C(O) H, —NH₂, —NHR, —NR₂—NO₂, —COOH, —CONH₂, —NH—C(O)—O—C₁₋₆ alkyl, C₁₋₆ alkyl, —C(O)NH—C₁₋₆ alkyl, —C(O)—C₁₋₆ alkyl. —O—C(O)—C₁₋₆ alkyl, or —C(O)—OC₁₋₆ alkyl, wherein the C₁₋₆ alkyl is optionally substituted. In some embodiments, m is 0-5. In some embodiments, m is 0-4. In some embodiments, m is 0-3. In some embodiments, m is 0-2. In some embodiments, m is 0-1. In some embodiments, m is 1-5. In some embodiments, m is 1-4. In some embodiments, m is 1-3. In some embodiments, m is 1-2. In some embodiments, m is 2-5. In some embodiments, m is 2-4. In some embodiments, m is 2-3. In some embodiments, m is 3-5. In some embodiments, m is 3-4. In some embodiments, m is 4-5. In some embodiments, m is 0. In some embodiments, m is 1. In some embodiments, m is 2. In some embodiments, m is 3. In some embodiments, m is 4. In some embodiments, m is 5.

In some embodiments, R¹² and R¹³ are each, independently hydrogen, halo, C₁₋₆ alkyl, C₁₋₆ haloalkyl, —OH, —O—(C₁₋₆ alkyl), —NR₂, —CN, oxo, —NO₂, —(C₁₋₆ alkyl)-O—(C₁₋₆ alkyl), —(C₁₋₆ alkyl)-O—(C₁₋₆ alkyl)-O—(C₁₋₆ alkyl), —NRC(═O)OR, —C(═O)R—C(═O)OR, —OC(═O)R, —C(═O)NR₂, —NHC(═O)R, C₂₋₆ alkene, phenyl, —S(═O)R, —S(═O)₂R, or a 3-7 membered monocyclic carbocyclic ring. In some embodiments, R¹² and R¹³ are each, independently hydrogen, —F, —Cl, —Br, —CH₃, —CH—CH₃, —CH(CH₃)₂, —OH, —OMe, —NH₂, —NH(CH₃), —N(CH₃)₂, —N(CH₂CH₃)₂, —N(CH₂CH₂CH₃)₂, —CN, —NO₂, —CF₃, ═O, —CH₂OCH₃, —CH₂OCH₂OCH₃, —OC(═O)Me, —C(═O)OH, —C(═O)OMe, —C(═O)OtBu, —C(—O)NH₂, —C(═O)NHCH₃, —C(═O)N(CH₃)₂, —NHC(═O)Me, —NHC(═O)OtBu, —CH—CH₂, phenyl, —S(═O)₂Me, —S(═O)₂tBu, —S(═O)₂(cyclopropyl), or cyclopropyl. In some embodiments, R¹² is —OH, halogen, —CN, —C(O) H, —NH₂, —NO₂, —COOH, —CONH₂, —NH—C(O)—O—C₁₋₆ alkyl, C₁₋₆ alkyl, —C(O)NH—C₁₋₆ alkyl, —C(O)—C₁₋₆ alkyl, —O—C(O)—C₁₋₆ alkyl, or —C(O)—OC₁₋₆ alkyl, wherein the C₁₋₆ alkyl is optionally substituted. In some embodiments, R¹² is absent. In some embodiments, R¹² is H. In some embodiments, R¹² is —OH. In some embodiments, R¹² is a halogen. In some embodiments, R¹² is —F. In some embodiments, R¹² is bromo. In some embodiments, R¹² is chloro. In some embodiments, R¹² is fluoro. In some embodiments, R¹² is —CN. In some embodiments, R¹² is —C(O) H. In some embodiments, R¹² is —NR₂. In some embodiments, R¹² is —NH₂. In some embodiments, R¹² is —NH(CH₃). In some embodiments, R¹² is —N(CH₃)₂. In some embodiments, R¹² is —N(CH₂CH₃)₂. In some embodiments, R¹² is —N(CH₂CH₂CH₃)₂. In some embodiments, R¹² is ═O. In some embodiments, R¹² is —NO₂. In some embodiments, R¹² is —CN. In some embodiments, R¹² is —COOH. In some embodiments, R¹² is —CONH₂. In some embodiments, R¹² is —NR—C(O)—OR. In some embodiments, R¹² is —NH—C(O)—O—C₁₋₆ alkyl. In some embodiments, R¹² is —NHC(═O)OMe. In some embodiments, R¹² is —NHC(═O)OtBu. In some embodiments, R¹² is C₁₋₆ alkyl. In some embodiments, R¹² is methyl. In some embodiments, R¹² is ethyl. In some embodiments, R¹² is isopropyl. In some embodiments, R¹² is C₁₋₆ haloalkyl. In some embodiments, R¹² is CF₃. In some embodiments. R¹² is —O—(C₁₋₆ alkyl). In some embodiments, R¹² is —OMe. In some embodiments, R¹² is —OEt. In some embodiments, R¹² is —(C₁₋₆ alkyl)-O—(C₁₋₆ alkyl). In some embodiments, R¹² is —CH₂OCH₃. In some embodiments, R¹² is —(C₁₋₆ alkyl)-O—(C₁₋₆ alkyl)-O—(C₁₋₆ alkyl). In some embodiments, R¹² is CH₂OCH₂OCH₃. In some embodiments, R¹² is —C(O)NR₂. In some embodiments, R¹² is —C(═O)N(CH₃)₂. In some embodiments, R¹² is —C(O)NH—C₁₋₆ alkyl. In some embodiments, R¹² is —C(═O)NHCH₃. In some embodiments, R¹² is —NHC(—O)R. In some embodiments R¹² is —NHC(═O))Me. In some embodiments, R¹² is —COR. In some embodiments, R¹² is —COH. In some embodiments, R¹² is —C(O)—C₁₋₆ alkyl. In some embodiments, R¹² is —C(═O) CH₃. In some embodiments, R¹² is —O—C(O)R. In some embodiments, R¹² is —O—C(O)—C₁₋₆ alkyl. In some embodiments, R¹² is —OC(═O)Me. In some embodiments, R¹² is —C(O))—OR. In some embodiments, R¹² is —C(O)—OC₁₋₆ alkyl. In some embodiments, R¹² is —C(═O)OMe. In some embodiments, R¹² is —C(═O)OtBu. In some embodiments, R¹² is C₂₋₆ alkene. In some embodiments, R¹² is —CH—CH₂. In some embodiments, R¹² is phenyl. In some embodiments, R¹² is —S(═O)R. In some embodiments. R¹² is —S(═O)₂R. In some embodiments, R¹² is S(═O)₂Me. In some embodiments, R¹² is —S(═O)₂tBu. In some embodiments, R¹² is S(═O)₂(cyclopropyl). In some embodiments, R¹² is cyclopropyl. In some embodiments, R¹² is —NHR.

In some embodiments, R¹³ is —OH, halogen, —CN, —C(O) H, —NH₂, —NO₂, —COOH, —CONH₂, —NH—C(O)—O—C₁₋₆ alkyl, C₁₋₆ alkyl, —C(O)NH—C₁₋₆ alkyl, —C(O)—C₁₋₆ alkyl, —O—C(O)—C₁₋₆ alkyl, or —C(O)—OC₁₋₆ alkyl, wherein the C₁₋₆ alkyl is optionally substituted. In some embodiments, R¹³ is absent. In some embodiments, R¹³ is hydrogen, optionally substituted C₁₋₆ alkyl, —OH, or halogen. In some embodiments, R¹³ is hydrogen, optionally substituted C₁₋₆ alkyl, —OH, or fluoro. In some embodiments, R¹³ is H. In some embodiments, R¹³ is C₁₋₆ alkyl. In some embodiments, R¹³ is methyl. In some embodiments, R¹³ is ethyl. In some embodiments, R¹³ is —OH. In some embodiments, R¹³ is halogen. In some embodiments, R¹³ is —CN. In some embodiments, R¹³ is —C(O) H. In some embodiments, R¹³ is —NH₂. In some embodiments, R¹³ is —NO₂. In some embodiments, R¹³ is —COOH. In some embodiments, R¹³ is —CONH₂. In some embodiments, R¹³ is —NH—C(O)—O—C₁₋₆ alkyl. In some embodiments, R¹³ is C₁₋₆ alkyl. In some embodiments, R¹³ is —C(O)NH—C₁₋₆ alkyl. In some embodiments, R¹³ is —C(O)—C₁₋₆ alkyl. In some embodiments, R¹³ is —O—C(O)—C₁₋₆ alkyl. In some embodiments, R¹³ is —C(O)—OC₁₋₆ alkyl. In some embodiments, the C₁₋₆ alkyl is optionally substituted as described and provided herein. In some embodiments, R¹³ is —NHR. In some embodiments, R¹³ is —NR₂, R is as defined and provided for herein.

In some embodiments, provided are compounds as described and provided for herein, wherein R¹⁵ is H, D, halo, oxo, C₁₋₆ alkyl, C₂₋₆ alkenyl, C₂₋₆ alkynyl, C₁₋₆ haloalkyl, C₃₋₆ cycloalkyl, CN, NO₂, C(O)NR₂, C(O)OR, or B(OR)₂. In some embodiments, R¹⁵ is H. In some embodiments, R¹⁵ is D. In some embodiments, R¹⁵ is halo. In some embodiments, R¹⁵ is oxo. In some embodiments, R¹⁵ is C₁₋₆ alkyl. In some embodiments, R¹⁵ is C₂₋₆ alkenyl. In some embodiments, R¹⁵ is C₂₋₆ alkynyl. In some embodiments, R¹⁵ is C₁₋₆ haloalkyl. In some embodiments, R¹⁵ is C₃₋₆ cycloalkyl. In some embodiments, R¹⁵ is CN. In some embodiments, R¹⁵ is NO₂. In some embodiments, R¹⁵ is C(O)NR₂. In some embodiments, R¹⁵ is C(O)OR. In some embodiments, R¹⁵ is B(OR)₂.

In some embodiments, provided are compounds as described and provided for herein, wherein R^{15′} is H, D, halo, oxo, C₁₋₆ alkyl, C₂₋₆ alkenyl, C₂₋₆ alkynyl, C₁₋₆ haloalkyl, C₃₋₆ cycloalkyl, CN, NO₂, C(O)NR₂, C(O)OR, or B(OR)₂. In some embodiments, R^{15′} is H. In some embodiments, R^{15′} is D. In some embodiments, R^{15′} is halo. In some embodiments, R^{15′} is oxo. In some embodiments, R^{15′} is C₁₋₆ alkyl. In some embodiments, R^{15′} is C₂₋₆ alkenyl. In some embodiments, R^{15′} is C₂₋₆ alkynyl. In some embodiments, R^{15′} is C₁₋₆ haloalkyl. In some embodiments, R^{15′} is C₃₋₆ cycloalkyl. In some embodiments, R^{15′} is CN. In some embodiments, R^{15′} is NO₂. In some embodiments, R^{15′} is C(O)NR₂. In some embodiments, R^{15′} is C(O)OR. In some embodiments, R^{15′} is B(OR)₂.

In some embodiments, R¹⁵ and R^{15′} are independently selected from those depicted in Table A. Table B. or Table C. In some embodiments, R¹⁵ and R^{15′} are both hydrogen. In some embodiments, R¹⁵ and R^{15′} are both methyl. In some embodiments, R¹⁵ is methyl and R¹⁵ is hydrogen.

In some embodiments, provided are compounds as described and provided for herein, wherein p is 0-8. In some embodiments, p is 0-7. In some embodiments, p is 0-6. In some embodiments, p is 0-5. In some embodiments, p is 0-4. In some embodiments, p is 0-3. In some embodiments, p is 0-2. In some embodiments, p is 0-1. In some embodiments, p is 1-8. In some embodiments, p is 1-7. In some embodiments, p is 1-6. In some embodiments, p is 1-5. In some embodiments, p is 1-4. In some embodiments, p is 1-3. In some embodiments, p is 1-2. In some embodiments, p is 2-8. In some embodiments, p is 2-7. In some embodiments, p is 2-6. In some embodiments, p is 2-5. In some embodiments, p is 2-4. In some embodiments, p is 2-3. In some embodiments, p is 3-8. In some embodiments, p is 3-7. In some embodiments, p is 3-6. In some embodiments, p is 3-5. In some embodiments, p is 3-4. In some embodiments, p is 4-8. In some embodiments, p is 4-7. In some embodiments, p is 4-6. In some embodiments, p is 4-5. In some embodiments, p is 5-8. In some embodiments, p is 5-7. In some embodiments, p is 5-6. In some embodiments, p is 6-8. In some embodiments, p is 6-7. In some embodiments, p is 7-8. In some embodiments, p is 0. In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8.

In some embodiments, R² is R¹⁴.

In some embodiments, R² is optionally substituted adamantyl.

In some embodiments, R² is selected from

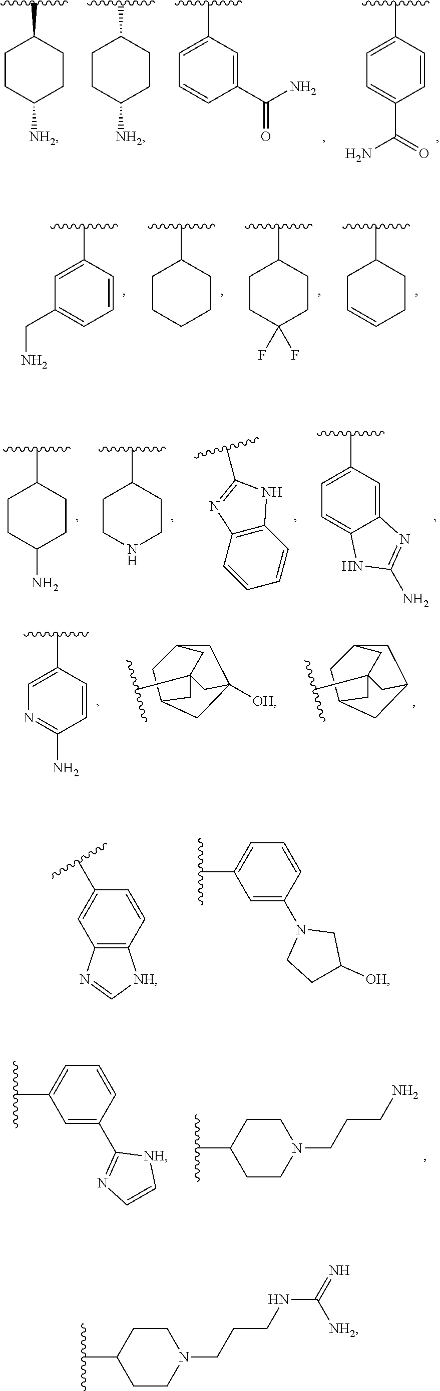

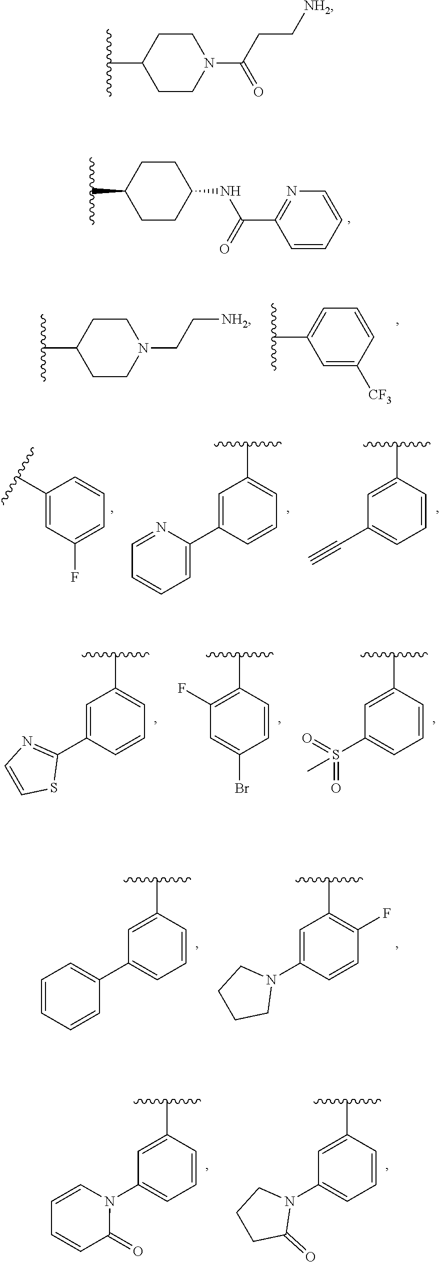

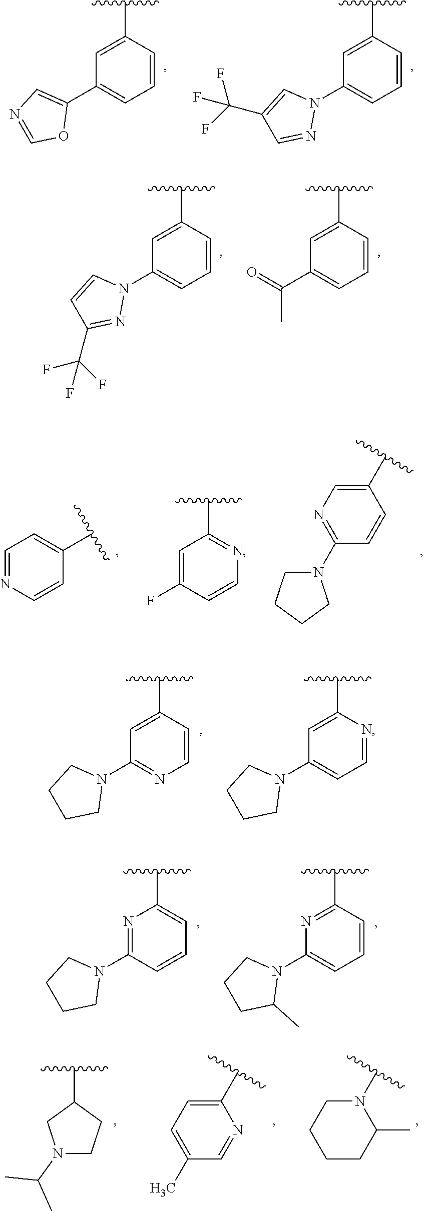

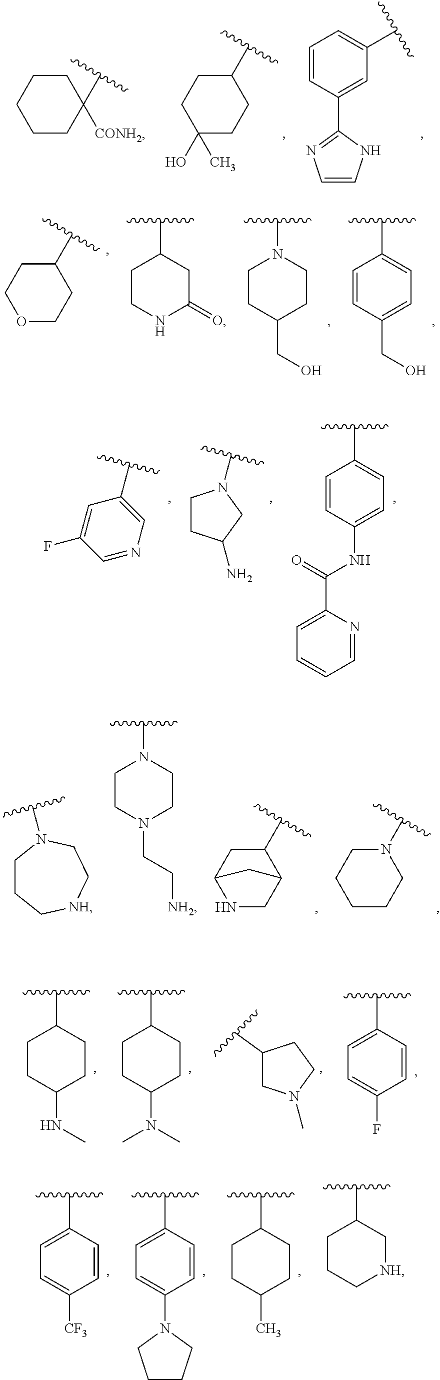

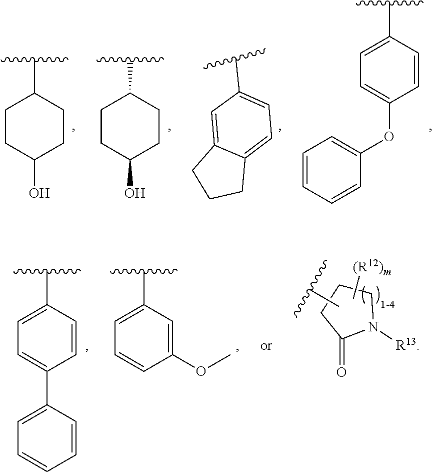

In some embodiments. R¹ is selected from

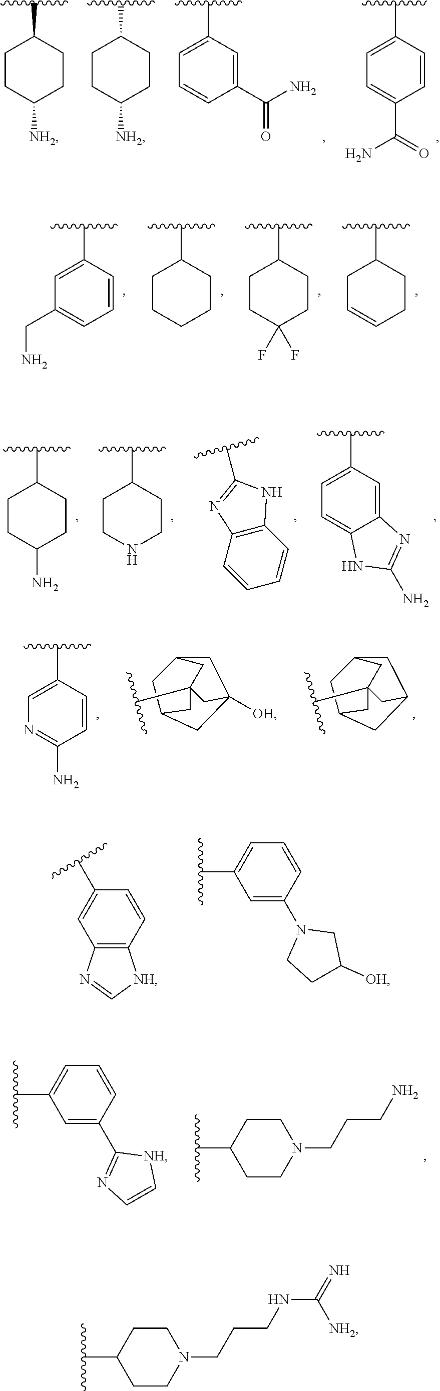

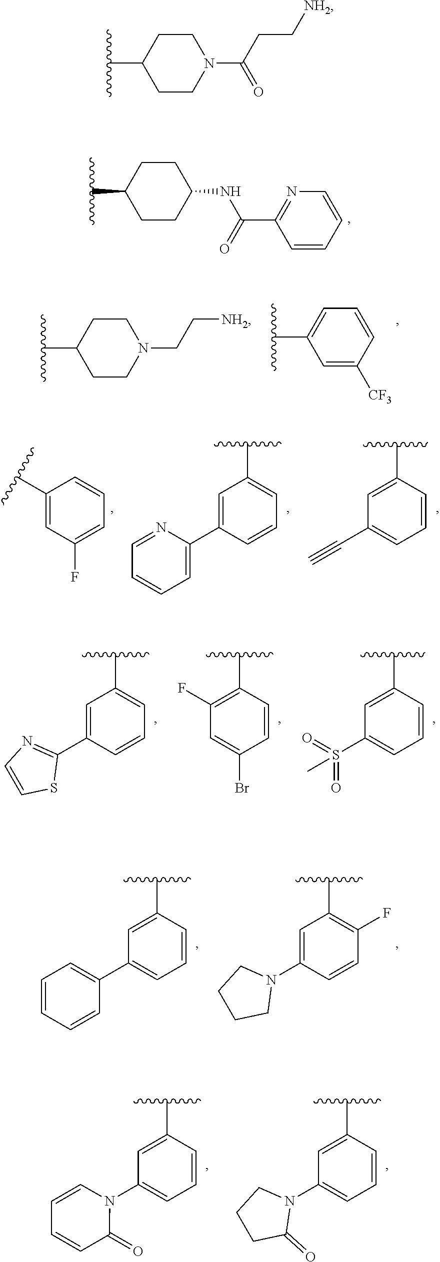

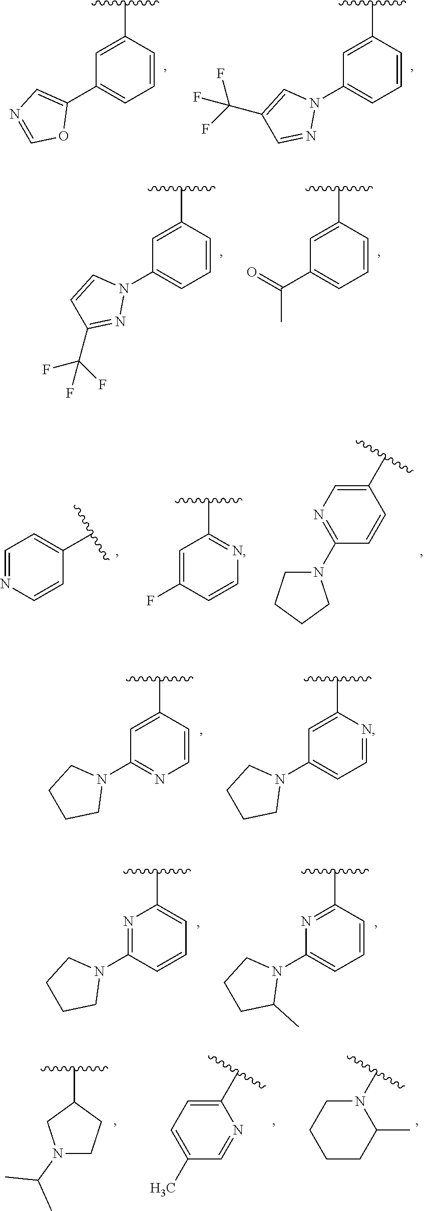

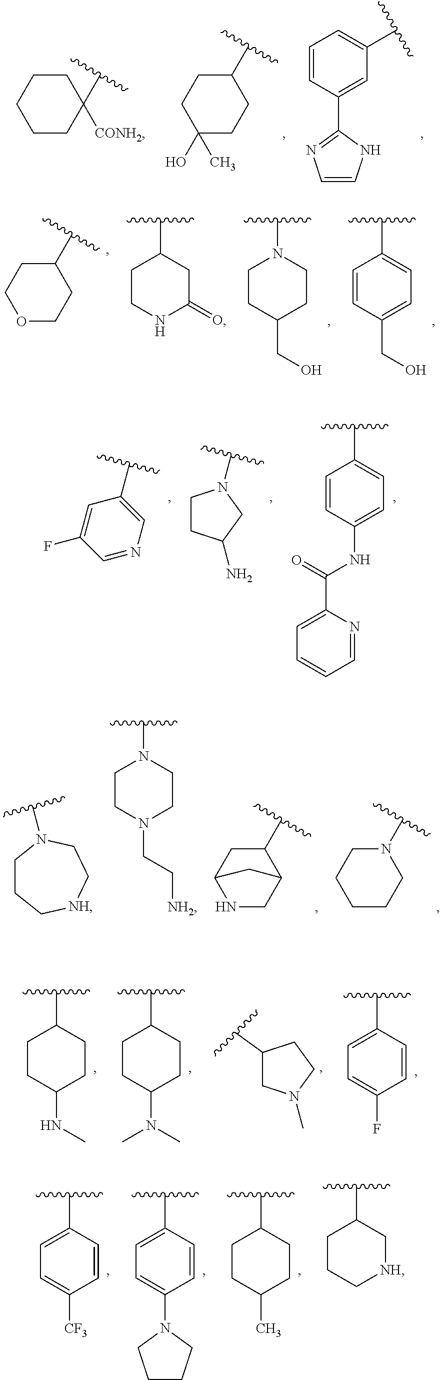

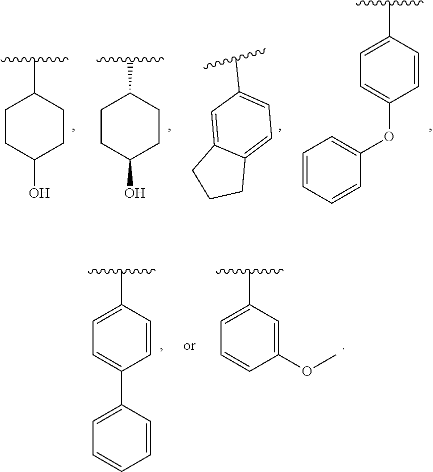

In some embodiments, R² is

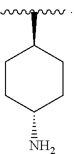 [SMILES: *[C@H]1CC[C@H](N)CC1]

In some embodiments, R² is

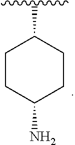 [SMILES: *[C@H]1CC[C@@H](N)CC1]

In some embodiments, R² is

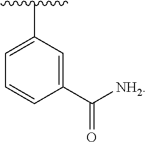 [SMILES: *c1cccc(C(N)=O)c1]

In some embodiments, R² is

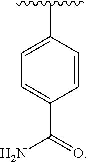 [SMILES: *c1ccc(C(N)=O)cc1]

In some embodiments, R² is

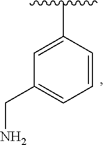 [SMILES: *c1cccc(CN)c1]

In some embodiments, R² is

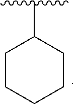 [SMILES: *C1CCCCC1]

In some embodiments, R² is

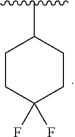 [SMILES: *C1CCC(F)(F)CC1]

In some embodiments, R² is

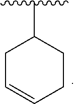 [SMILES: *C1CC=CCC1]

In some embodiments, R² is

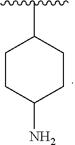 [SMILES: *C1CCC(N)CC1]

In some embodiments, R² is

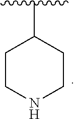 [SMILES: *C1CCNCC1]

In some embodiments, R² is

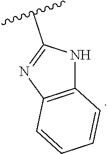 [SMILES: *c1nc2ccccc2[nH]1]

In some embodiments, R² is

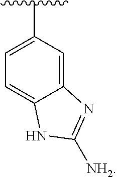 [SMILES: *c1ccc2[nH]c(N)nc2c1]

In some embodiments, R² is

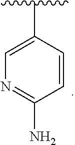 [SMILES: *c1ccc(N)nc1]

In some embodiments, R² is

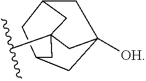 [SMILES: *C12CC3CC(C1)CC(O)(C3)C2]

In some embodiments, R² is

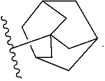 [SMILES: *C12CC3CC(CC(C3)C1)C2]

In some embodiments, R² is

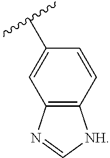 [SMILES: *c1ccc2[nH]cnc2c1]

In some embodiments, R² is

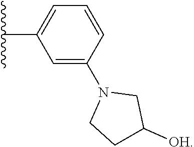 [SMILES: *c1cccc(N2CCC(O)C2)c1]

In some embodiments, R² is

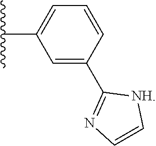 [SMILES: *c1cccc(-c2ncc[nH]2)c1]

In some embodiments, R² is

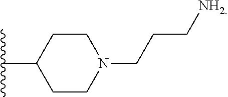 [SMILES: *C1CCN(CCCN)CC1]

In some embodiments, R² is

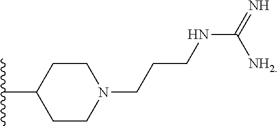 [SMILES: *C1CCN(CCCNC(=N)N)CC1]

In some embodiments, R² is

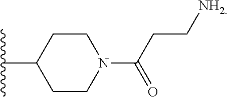 [SMILES: *C1CCN(C(=O)CCN)CC1]

In some embodiments, R² is

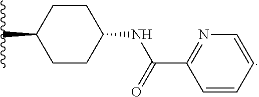 [SMILES: *[C@H]1CC[C@H](NC(=O)c2ccccn2)CC1]

In some embodiments, R² is

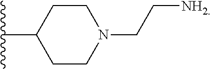 [SMILES: *C1CCN(CCN)CC1]

In some embodiments, R² is

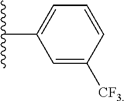 [SMILES: *c1cccc(C(F)(F)F)c1]

In some embodiments, R² is

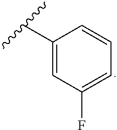 [SMILES: *c1cccc(F)c1]

In some embodiments, R² is

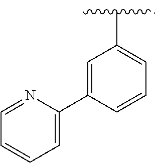 [SMILES: *c1cccc(-c2ccccn2)c1]

In some embodiments, R² is

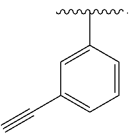 [SMILES: *c1cccc(C#C)c1]

In some embodiments, R² is

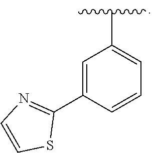 [SMILES: *c1cccc(-c2nccs2)c1]

In some embodiments, R² is

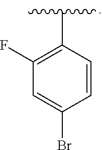 [SMILES: *c1ccc(Br)cc1F]

In some embodiments, R² is

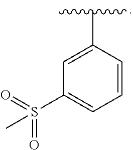 [SMILES: *c1cccc(S(C)(=O)=O)c1]

In some embodiments, R² is

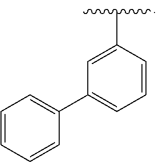 [SMILES: *c1cccc(-c2ccccc2)c1]

In some embodiments, R² is

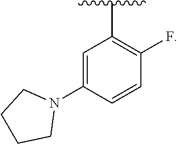 [SMILES: *c1cc(N2CCCC2)ccc1F]

In some embodiments, R² is

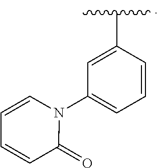 [SMILES: *c1cccc(-n2ccccc2=O)c1]

In some embodiments, R² is

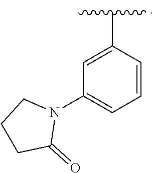 [SMILES: *c1cccc(N2CCCC2=O)c1]

In some embodiments, R² is

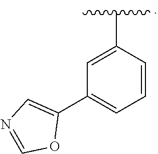 [SMILES: *c1cccc(-c2cnco2)c1]

In some embodiments, R² is

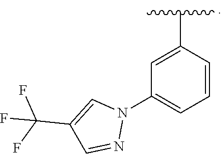 [SMILES: *c1cccc(-n2cc(C(F)(F)F)cn2)c1]

In some embodiments, R² is

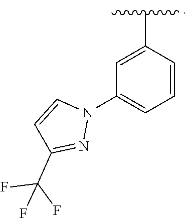 [SMILES: *c1cccc(-n2ccc(C(F)(F)F)n2)c1]

In some embodiments, R² is

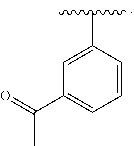 [SMILES: *c1cccc(C(C)=O)c1]

In some embodiments, R² is

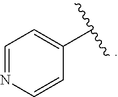 [SMILES: *c1ccncc1]

In some embodiments, R² is

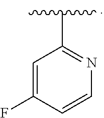 [SMILES: *c1cc(F)ccn1]

In some embodiments, R² is

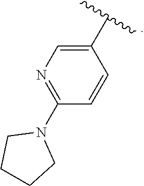 [SMILES: *c1ccc(N2CCCC2)nc1]

In some embodiments, R² is

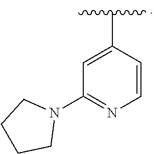 [SMILES: *c1ccnc(N2CCCC2)c1]

In some embodiments, R² is

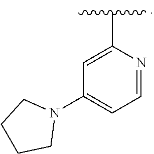 [SMILES: *c1cc(N2CCCC2)ccn1]

In some embodiments, R² is

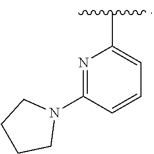 [SMILES: *c1cccc(N2CCCC2)n1]

In some embodiments, R² is

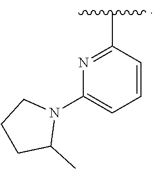 [SMILES: *c1cccc(N2CCCC2C)n1]

In some embodiments, R² is

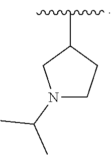 [SMILES: *C1CCN(C(C)C)C1]

In some embodiments, R² is

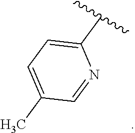 [SMILES: *c1ccc(C)cn1]

In some embodiments, R² is

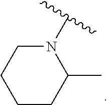 [SMILES: *N1CCCCC1C]

In some embodiments, R² is

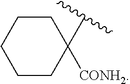 [SMILES: *C1(C(N)=O)CCCCC1]

In some embodiments, R² is

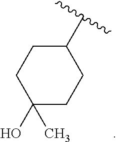 [SMILES: *C1CCC(C)(O)CC1]

In some embodiments, R² is

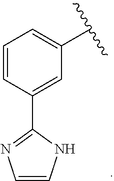 [SMILES: *c1cccc(-c2ncc[nH]2)c1]

In some embodiments, R² is

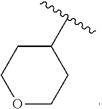 [SMILES: *C1CCOCC1]

In some embodiments, R² is

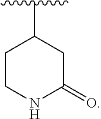 [SMILES: *C1CCNC(=O)C1]

In some embodiments, R² is

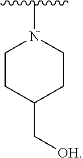 [SMILES: *N1CCC(CO)CC1]

In some embodiments, R² is

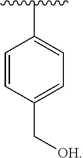 [SMILES: *c1ccc(CO)cc1]

In some embodiments, R² is

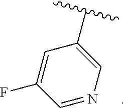 [SMILES: *c1cncc(F)c1]

In some embodiments, R² is

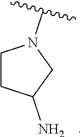 [SMILES: *N1CCC(N)C1]

In some embodiments, R² is

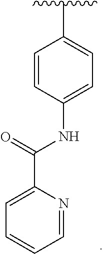 [SMILES: *c1ccc(NC(=O)c2ccccn2)cc1]

In some embodiments, R² is

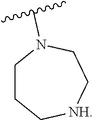 [SMILES: *N1CCCNCC1]

In some embodiments, R² is

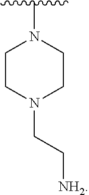 [SMILES: *N1CCN(CCN)CC1]

In some embodiments, R² is

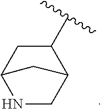 [SMILES: *C1CC2CC1CN2]

In some embodiments, R² is

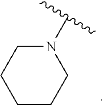 [SMILES: *N1CCCCC1]

In some embodiments, R² is

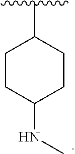 [SMILES: *C1CCC(NC)CC1]

In some embodiments, R² is

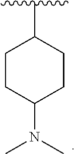 [SMILES: *C1CCC(N(C)C)CC1]

In some embodiments, R² is

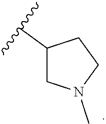 [SMILES: *C1CCN(C)C1]

In some embodiments, R² is

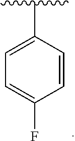 [SMILES: *c1ccc(F)cc1]

In some embodiments, R² is

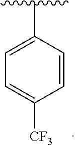 [SMILES: *c1ccc(C(F)(F)F)cc1]

In some embodiments, R² is

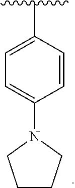 [SMILES: *c1ccc(N2CCCC2)cc1]

In some embodiments, R² is

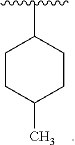 [SMILES: *C1CCC(C)CC1]

In some embodiments, R² is

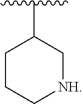 [SMILES: *C1CCCNC1]

In some embodiments, R² is

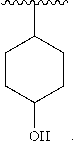 [SMILES: *C1CCC(O)CC1]

In some embodiments, R² is

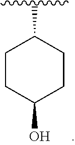 [SMILES: *[C@H]1CC[C@H](O)CC1]

In some embodiments, R² is

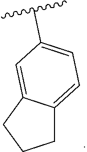 [SMILES: *c1ccc2c(c1)CCC2]

In some embodiments, R² is

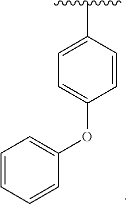 [SMILES: *c1ccc(Oc2ccccc2)cc1]

In some embodiments, R² is

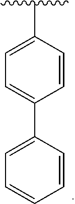 [SMILES: *c1ccc(-c2ccccc2)cc1]

In some embodiments, R² is

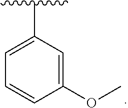 [SMILES: *c1cccc(OC)c1]

In some embodiments, R² is

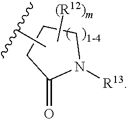

In some embodiments, R² is selected from those depicted in Table A, Table B, or table C.

In some embodiments, -L²-R² is —C(═O)—O—(C₁₋₆ alkyl)-(Ring A), —C(═O)—NH—(C₁₋₆ alky)-(Ring A), or —C(═O))—(NCH₃)—(C₁₋₆ alky)-(Ring A), wherein Ring A is optionally substituted and selected from phenyl; a 5-6 membered heteroaromatic ring having 1-4 heteroatoms independently selected from nitrogen, oxygen, and sulfur; a 3-7 membered monocyclic carbocyclic ring: a 4-7 membered heterocyclic ring having 1-3 heteroatoms independently selected from nitrogen, oxygen, and sulfur; a 5-10 membered bicyclic carbocyclic ring: a 7-10 membered bicyclic heterocyclic ring having 1-4 heteroatoms independently selected from nitrogen, oxygen, and sulfur; an 8-10 membered bicyclic aromatic carbocyclic ring; and an 8-10 membered bicyclic heterocyclic ring having 1-3 heteroatoms independently selected from nitrogen, oxygen, and sulfur; and wherein the C₁₋₆ alky is optionally substituted. In some embodiments, -L²-R² is —C(═O)—O—(C₁₋₆ alkyl)-(Ring A), —C(═O)—NH—(C₁₋₆ alky)-(Ring A), or —C(═O)—(NCH₃)—(C₁₋₆ alky)-(Ring A), wherein Ring A is optionally substituted and selected from phenyl. In some embodiments, -L²-R² is —C(═O)—O—(C₁₋₆ alkyl)-(Ring A), —C(═O)—NH—(C₁₋₆ alky)-(Ring A), or —C(═O)—(NCH₃)—(C₁₋₆ alky)-(Ring A), wherein Ring A is optionally substituted and selected from a 5-6 membered heteroaromatic ring having 1-4 heteroatoms independently selected from nitrogen, oxygen, and sulfur. In some embodiments, -L²-R² is —C(═O)—O—(C₁₋₆ alkyl)-(Ring A), —C(═O)—NH—(C₁₋₆ alky)-(Ring A), or —C(═O)—(NCH₃)—(C₁₋₆ alky)-(Ring A), wherein Ring A is optionally substituted and selected from a 4-7 membered heterocyclic ring having 1-3 heteroatoms independently selected from nitrogen, oxygen, and sulfur. In some embodiments, -L²-R² is —C(═O)—O—(C₁₋₆ alkyl)-(Ring A), —C(═O)—NH—(C₁₋₆ alky)-(Ring A), or —C(═O)—(NCH₃)—(C₁₋₆ alky)-(Ring A), wherein Ring A is optionally substituted and selected from a 4-7 membered heterocyclic ring having 1-3 heteroatoms independently selected from nitrogen, oxygen, and sulfur, wherein Ring A is substituted with methyl, fluoro, or oxo. In some embodiments, Ring A is

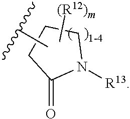

In some embodiments, -L²-R² is selected from those depicted in Table A, Table B. or table C.

In some embodiments, provided are compounds as described and provided for herein, wherein R³ is absent, hydrogen, —OH, halogen, —CN, —C(O) H, —NH₂, —NO₂, —COOH, —CONH₂, —NH—C(O)—O—C₁₋₆ alkyl, C₁₋₆ alkyl, or —C(O)—C₁₋₆ alkyl, wherein the C₁₋₆ alkyl is optionally substituted. In some embodiments, provided are compounds as described and provided for herein, wherein R³ is absent, —OH, halogen, —CN, —C(O) H, —NH₂, —NO₂, —COOH, —CONH₂, —NH—C(O)—O—C₁₋₆ alkyl, C₁₋₆ alkyl, or —C(O)—C₁₋₆ alkyl, wherein the C₁₋₆ alkyl is optionally substituted. In some embodiments, R³ is absent. In some embodiments, R³ is hydrogen. In some embodiments, R³ is —OH. In some embodiments, R³ is halogen. In some embodiments, R³ is —CN. In some embodiments, R³ is —C(O) H. In some embodiments, R³ is —NH₂. In some embodiments, R³ is —NO₂. In some embodiments, R³ is —COOH. In some embodiments, R³ is —CONH₂. In some embodiments, R³ is —NH—C(O)—O—C₁₋₆ alkyl. In some embodiments, R³ is C₁₋₆ alkyl. In some embodiments, R³ is or —C(O)—C₁₋₆ alkyl. In some embodiments, the C₁₋₆ alkyl is optionally substituted as described and provided for herein.

In some embodiments, R³ is selected from those depicted in Table A. Table B, or Table C.

In some embodiments, provided are compounds as described and provided for herein, wherein L³ is a bond, or an optionally substituted bivalent C₁₋₈ saturated or unsaturated, straight or branched hydrocarbon chain, wherein 1, 2, or 3 methylene units of the C(O)—O—, —O—C(O)—, —NR—S(O)₂—, or —S(O)₂—NR—. In some embodiments, L³ is a bond. In some embodiments, L³ is an optionally substituted bivalent C₁₋₈ saturated or unsaturated. In some embodiments, L³ is straight or branched hydrocarbon chain. In some embodiments, L³ is a bivalent C₁ chain, wherein the 1 methylene unit is optionally replaced by —NR—C(O)—, —C(O)—NR—, —C(O)—, —S(O)₂—, —C(O)—O—, —O—C(O)—, —NR—S(O)₂—, or —S(O)₂—NR—. In some embodiments, L³ is a bivalent C₁ chain, wherein the 1 methylene unit is replaced by —NR—C(O)—, —C(O)—NR—, —C(O)—, —S(O)—, —C(O)—O)—, —O—C(O)—, —NR—S(O))₂—, or —S(O)₂—NR—. In some embodiments, L³ is wherein 1. In some embodiments, L³ is 2. In some embodiments, L³ is 3 methylene units of the hydrocarbon chain are optionally replaced by —NR—C(O)—. In some embodiments, L³ is —C(O)—NR—. In some embodiments, L³ is —C(O)—. In some embodiments. L³ is —S(O)₂—. In some embodiments, L³ is —C(O)—O—. In some embodiments, L³ is —O—C(O)—. In some embodiments, L³ is —NR—S(O)₂—. In some embodiments, L³ is —S(O)₂—NR—. In some embodiments, L³ is —CH₂—.

In some embodiments, L³ is selected from those depicted in Table A, Table B, or Table C.

In some embodiments, R⁴ is —NH₂, NHR, —N═C═N—, —NR₂, —CH₂—NH—C═N, —PO₄H₂, or —H. In some embodiments, provided are compounds as described and provided for herein, wherein R⁴ is —NH₂, NHR, —N═C═N—, or —NR₂. In some embodiments, R⁴ is —NR₂. In some embodiments, R⁴ is —NH₂. In some embodiments, R⁴ is —N═C═N—. In some embodiments. R⁴ is NHR. In some embodiments. R⁴ is —CH₂—NH—C═N. In some embodiments, R⁴ is —PO₄H₂. In some embodiments, R⁴ is —H.

In some embodiments, R⁴ is selected from those depicted in Table A, Table B, or Table C.

In some embodiments, each R is, independently, H, —OH, —C₁₋₈ alkyl, —C₁₋₈ alkyl(3-7 membered monocyclic carbocyclyl), —OC₁₋₈ alkyl, —C(O)—C₁₋₈ alkyl, —O—C(O)—C₁₋₈ alkyl, C₁₋₈ alkyl-O—C(O)—C₁₋₈ alkyl, —C(O)—OC₁₋₈ alkyl, 3-7 membered monocyclic carbocyclyl, —O-(4-7 membered monocyclic carbocyclyl), —C(O)-(4-7 membered monocyclic carbocyclyl), —C(O)—O-(4-7 membered monocyclic carbocyclyl), phenyl, —O-phenyl, —C(O)-phenyl, —C(O)—O— phenyl, —C(O)-phenyl-(C₁₋₈ alkyl)-O—C(O)—O—(C₁₋₈ alkyl)-, —C(O)—O—(C₁₋₈ alkyl)-(4-7 membered monocyclic heterocyclic ring), 8-10 membered bicyclic aryl, —O-(8-10 membered bicyclic aryl), —C(O)-(8-10 membered bicyclic aryl), or —C(O)—O-(8-10 membered bicyclic aryl), wherein each of the C₁₋₈ alkyl, 4-7 membered monocyclic carbocyclyl, phenyl, and 8-10 membered bicyclic aryl is optionally and independently substituted. In some embodiments, provided are compounds as described and provided for herein, wherein each R is, independently. H, —OH, —C₁₋₈ alkyl, —OC₁₋₈ alkyl, —C(O)—C₁₋₈ alkyl, —C(O)—OC₁₋₈ alkyl, 4-7 membered monocyclic carbocyclyl, —O-(4-7 membered monocyclic carbocyclyl), —C(O)-(4-7 membered monocyclic carbocyclyl), —C(O)—O-(4-7 membered monocyclic carbocyclyl), phenyl, —O-phenyl, —C(O)-phenyl, —C(O)—O-phenyl, 8-10 membered bicyclic aryl, —O-(8-10 membered bicyclic aryl), —C(O)-(8-10 membered bicyclic aryl), or —C(O)—O-(8-10 membered bicyclic aryl), wherein each of the C₁₋₈ alkyl, 4-7 membered monocyclic carbocyclyl, phenyl, and 8-10 membered bicyclic aryl is optionally and independently substituted. In some embodiments, each R is, independently. H. In some embodiments, each R is, independently, —OH. In some embodiments, each R is, independently, —C₁₋₈ alkyl. In some embodiments. R is —C₁₋₈ alkyl(3-7 membered monocyclic carbocyclyl). In some embodiments, each R is, independently, —OC₁₋₈ alkyl. In some embodiments, each R is, independently, —C(O)—C₁₋₈ alkyl. In some embodiments, each R is, independently, —C(O)—OC₁₋₈ alkyl. In some embodiments, each R is, independently, —O—C(O)—C₁₋₈ alkyl. In some embodiments, each R is, independently, C₁₋₈ alkyl-O—C(O)—C₁₋₈ alkyl. In some embodiments, each R is, independently, 3-7 membered monocyclic carbocyclyl. In some embodiments, each R is, independently, 4-7 membered monocyclic carbocyclyl. In some embodiments, each R is, independently, —O-(4-7 membered monocyclic carbocyclyl). In some embodiments, each R is, independently, —C(O)-(4-7 membered monocyclic carbocyclyl). In some embodiments, each R is, independently, —C(O)—O-(4-7 membered monocyclic carbocyclyl). In some embodiments, each R is, independently, phenyl. In some embodiments, each R is, independently, —O-phenyl. In some embodiments, each R is, independently, —C(O)-phenyl. In some embodiments, each R is, independently, —C(O)-phenyl-(C₁₋₈ alkyl)-O—C(O)—O—(C₁₋₈ alkyl)-. In some embodiments, each R is, independently, —C(O)—O—(C₁₋₈ alkyl)-(4-7 membered monocyclic heterocyclic ring). In some embodiments, each R is, independently, —C(O)—O-phenyl. In some embodiments, each R is, independently, 8-10 membered bicyclic aryl. In some embodiments, each R is, independently, —O-(8-10 membered bicyclic aryl). In some embodiments, each R is, independently, —C(O)-(8-10 membered bicyclic aryl). In some embodiments, each R is, independently, —C(O)—O-(8-10 membered bicyclic aryl). In some embodiments, the C₁₋₈ alkyl is optionally and independently substituted as described and provided herein. In some embodiments, the 4-7 membered monocyclic carbocyclyl is optionally and independently substituted as described and provided herein. In some embodiments, the phenyl is optionally and independently substituted as described and provided herein. In some embodiments, the 8-10 membered bicyclic aryl is optionally and independently substituted as described and provided herein. In some embodiments, each of the C₁₋₈ alkyl, 4-7 membered monocyclic carbocyclyl, phenyl, and 8-10 membered bicyclic aryl is optionally and independently substituted with C₁₋₆ alkyl, halogen, —OH, —O—(C₁₋₆ alkyl), or —O. In some embodiments, each of the C₁₋₈ alkyl, 4-7 membered monocyclic carbocyclyl, phenyl, and 8-10 membered bicyclic aryl is optionally and independently substituted with —CH₃, —F, —Cl, —Br, —I, —OH, —OMe, or ═O. In some embodiments, R is selected from those depicted in Table A, Table B, or Table C.

In some embodiments, the compound of Formula I is a compound as provided throughout the present application. In some embodiments, the compound of Formula I is as provided in Table A, Table B, or Table C.

For Table A Kᵢ of Matriptase-2 values, (i) A indicates Kᵢ≤0.1 uM, (ii) B indicates 0.1<Kᵢ≤0.5 uM, and (iii) C indicates 0.5<Kᵢ<3 uM.

TABLE A

| | | | LCMS | HPLC | |
|---|---|---|---|---|---|
| | | | (M + H)+/ | Purity | Mat-2 |
| Cpmd # | Structure | IUPAC | [M − H]− | (%) | Kᵢ |
| 1 | 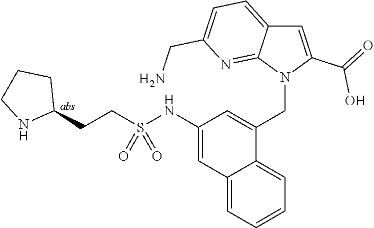 [SMILES: NCc1ccc2cc(C(=O)O)n(Cc3cc(NS(=O)(=O)CC[C@@H]4CCCN4)cc4ccccc34)c2n1] | 6-(aminomethyl)-1-[(3- [(2S)-pyrrolidin-2-yl]- ethanesulfonamido}- naphthalen-1-yl)methyl]- 1H-pyrrolo[2,3-b]pyr- idine-2-carboxylic acid | 508.3 | 98 | A |
| 2 | 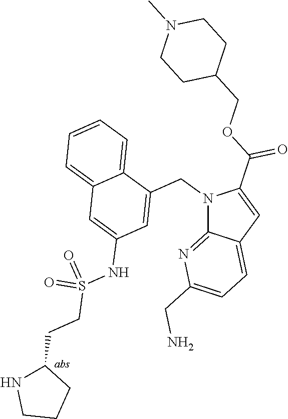 [SMILES: CN1CCC(COC(=O)c2cc3ccc(CN)nc3n2Cc2cc(NS(=O)(=O)CC[C@@H]3CCCN3)cc3ccccc23)CC1] | (1-methylpiperidin-4- yl)methyl 6-(amino- methyl)-1-[(3-{2-[(2S)- pyrrolidin-2-yl]ethane- sulfonamido}naphtha- len-1-yl)methyl]-1H- pyrrolo[2,3-b]pyridine- 2-carboxylate | 618.8 | 97 | C |
| 3 | 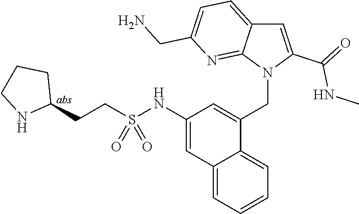 [SMILES: CNC(=O)c1cc2ccc(CN)nc2n1Cc1cc(NS(=O)(=O)CC[C@@H]2CCCN2)cc2ccccc12] | 6-(aminomethyl)-N- methyl-1-[(3-{2-[(2S)- pyrrolidin-2-yl]ethane- sulfonamido}naphtha- len-1-yl)methyl]-1H- pyrrolo[2,3-b]pyridine- 2-carboxamide | 521.3 | 100 | B |
| 4 | 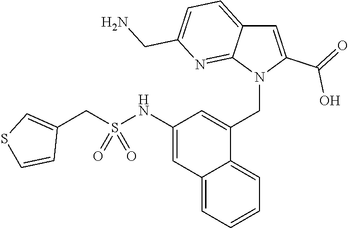 [SMILES: NCc1ccc2cc(C(=O)O)n(Cc3cc(NS(=O)(=O)Cc4ccsc4)cc4ccccc34)c2n1] | 6-(aminomethyl)-1-({3- [(thiophen-3-yl)methane- sulfonamido]naphtha- len-1-yl}methyl)-1H- pyrrolo[2,3-b]pyridine- 2-carboxylic acid | 507.0 | 100 | A |
| 5 | 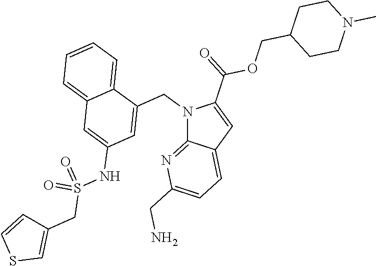 [SMILES: CN1CCC(COC(=O)c2cc3ccc(CN)nc3n2Cc2cc(NS(=O)(=O)Cc3ccsc3)cc3ccccc23)CC1] | (1-methylpiperidin-4- yl)-methyl 6-(amino- methyl)-1-({3-[(thio- phen-3-yl)methanesul- fonamido]naphthalen-1- yl}methyl)-1H-pyrrolo- [2,3-b]pyridine-2-car- boxylate | 616.2 [M − H] | 96 | A |
| 6 | 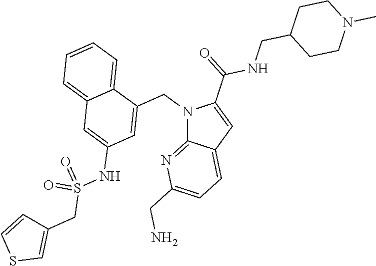 [SMILES: CN1CCC(CNC(=O)c2cc3ccc(CN)nc3n2Cc2cc(NS(=O)(=O)Cc3ccsc3)cc3ccccc23)CC1] | 6-(aminomethyl)-N-[(1- methylpiperidin-4-yl)- methyl]-1-({3-[(thio- phen-3-yl)methanesul- fonamido]naphthalen-1- yl}methyl)-1H-pyrrolo- [2,3-b]pyridine-2-car- boxamide | 615.2 [M − H] | 99 | A |
| 7 | 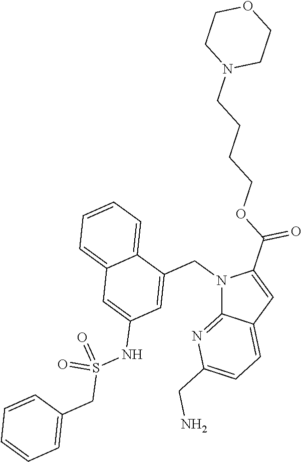 [SMILES: NCc1ccc2cc(C(=O)OCCCCN3CCOCC3)n(Cc3cc(NS(=O)(=O)Cc4ccccc4)cc4ccccc34)c2n1] | 4-(morpholin-4-yl)butyl 6-(aminomethyl)-1-{[3- (phenylmethanesulfon- amido)naphthalen-1-yl]- methyl}-1H-pyrrolo[2,3- b]pyridine-2-carboxylate | 642.0 | 100 | A |
| 8 | 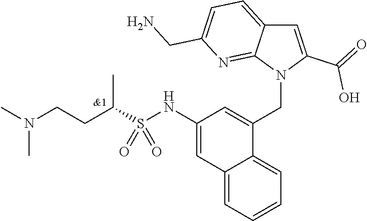 [SMILES: C[C@@H](CCN(C)C)S(=O)(=O)Nc1cc(Cn2c(C(=O)O)cc3ccc(CN)nc32)c2ccccc2c1] | rac-6-(aminomethyl)-1- ({3-[(2R)-4-(dimethyl- amino)butane-2-sulfon- amido]naphthalen-1-yl}- methyl)-1H-pyrrolo[2,3- b]pyridine-2-carboxylic acid | 510.0 | 98 | B |
| 9 | 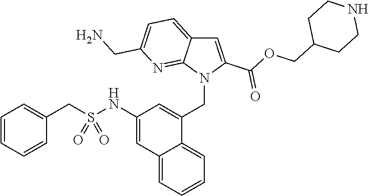 [SMILES: NCc1ccc2cc(C(=O)OCC3CCNCC3)n(Cc3cc(NS(=O)(=O)Cc4ccccc4)cc4ccccc34)c2n1] | (piperidin-4-yl)methyl 6-(aminomethyl)-1-{[3- (phenylmethanesulfon- amido)naphthalen-1-yl]- methyl}-1H-pyrrolo[2,3- b]pyridine-2-carboxylate | 598.4 | 100 | A |
| 10 | 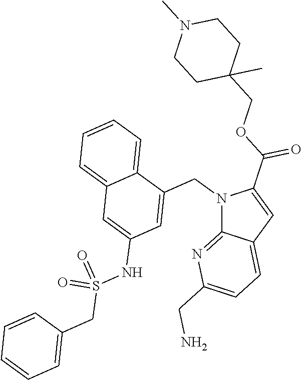 [SMILES: CN1CCC(C)(COC(=O)c2cc3ccc(CN)nc3n2Cc2cc(NS(=O)(=O)Cc3ccccc3)cc3ccccc23)CC1] | (1,4-dimethylpiperidin- 4-yl)methyl 6-(amino- methyl)-1-{[3-(phenyl- methanesulfonamido)- naphthalen-1-yl]meth- yl}-1H-pyrrolo[2,3-b]- pyridine-2-carboxylate | 626.5 | 97 | A |
| 11 | 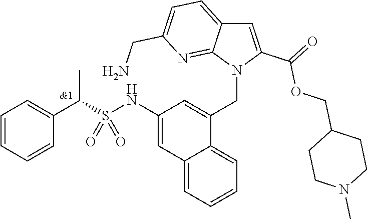 [SMILES: C[C@@H](c1ccccc1)S(=O)(=O)Nc1cc(Cn2c(C(=O)OCC3CCN(C)CC3)cc3ccc(CN)nc32)c2ccccc2c1] | (1-methylpiperidin-4- yl)methyl 6-(aminometh- yl)-1-{[3-(1-phenyl- ethanesulfonamido)naph- thalen-1-yl]methyl}-1H- pyrrolo[2,3-b]pyridine- 2-carboxylate | 625.8 | 99 | A |
| 12 | 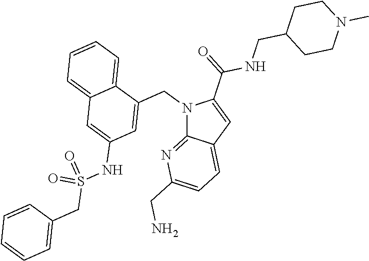 [SMILES: CN1CCC(CNC(=O)c2cc3ccc(CN)nc3n2Cc2cc(NS(=O)(=O)Cc3ccccc3)cc3ccccc23)CC1] | 6-(aminomethyl)-N-[(1- methylpiperidin-4-yl)- methyl]-1-{[3-(phenyl- methanesulfonamido)- naphthalen-1-yl]meth- yl}-1H-pyrrolo[2,3-b]- pyridine-2-carboxamide | 611.1 | 99 | A |
| 13 | 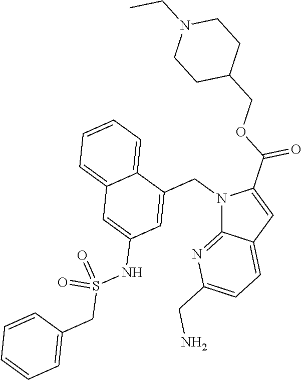 [SMILES: CCN1CCC(COC(=O)c2cc3ccc(CN)nc3n2Cc2cc(NS(=O)(=O)Cc3ccccc3)cc3ccccc23)CC1] | (1-ethylpiperidin-4-yl)- methyl 6-(aminomethyl)- 1-{[3-(phenylmethane- sulfonamido)naphthalen- 1-yl]methyl}-1H-pyr- rolo[2,3-b]pyridine-2- carboxylate | 626.0 | 100 | A |
| 14 | 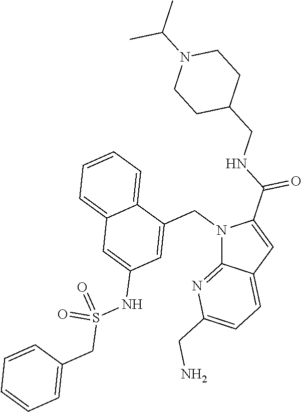 [SMILES: CC(C)N1CCC(CNC(=O)c2cc3ccc(CN)nc3n2Cc2cc(NS(=O)(=O)Cc3ccccc3)cc3ccccc23)CC1] | 6-(aminomethyl)-1-{[3- (phenylmethanesulfon- amido)naphthalen-1-yl]- methyl}-N-{[1-(propan- 2-yl)piperidin-4-yl]- methyl}-1H-pyrrolo- [2,3-b]pyridine-2-car- boxamide | 639.0 | 100 | A |
| 15 | 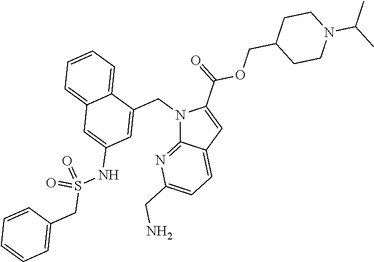 [SMILES: CC(C)N1CCC(COC(=O)c2cc3ccc(CN)nc3n2Cc2cc(NS(=O)(=O)Cc3ccccc3)cc3ccccc23)CC1] | [1-(propan-2-yl)piperi- din-4-yl]methyl 6- (aminomethyl)-1-{3- (phenylmethanesulfon- amido)naphthalen-1-yl]- methyl}-1H-pyrrolo[2,3- b]pyridine-2-carboxylate | 640.0 | 100 | A |
| 16 | 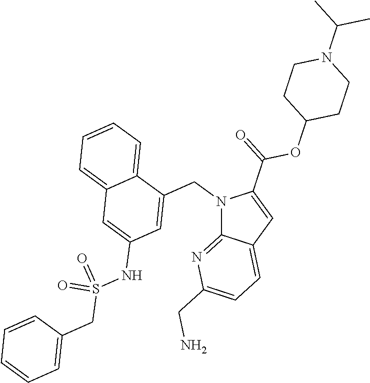 [SMILES: CC(C)N1CCC(OC(=O)c2cc3ccc(CN)nc3n2Cc2cc(NS(=O)(=O)Cc3ccccc3)cc3ccccc23)CC1] | 1-(propan-2-yl)piperidin- 4-yl 6-(aminomethyl)-1- {[3-(phenylmethane- sulfonamido)naphthalen- 1-yl]methyl}-1H-pyr- rolo[2,3-b]pyridine-2- carboxylate | 626.0 | 92 | A |
| 17 | 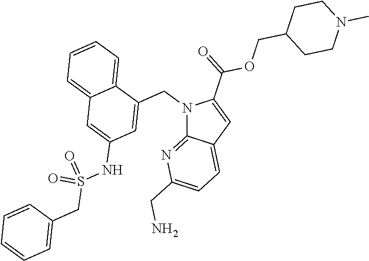 [SMILES: CN1CCC(COC(=O)c2cc3ccc(CN)nc3n2Cc2cc(NS(=O)(=O)Cc3ccccc3)cc3ccccc23)CC1] | (1-methylpiperidin-4- yl)methyl 6-(amino- methyl)-1-{[3-(phenyl- methanesulfonamido)- naphthalen-1-yl]methyl}- 1H-pyrrolo[2,3-b]pyr- idine-2-carboxylate | 612.4 | 99 | A |
| 18 | 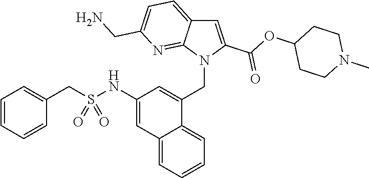 [SMILES: CN1CCC(OC(=O)c2cc3ccc(CN)nc3n2Cc2cc(NS(=O)(=O)Cc3ccccc3)cc3ccccc23)CC1] | 1-methylpiperidin-4-yl 6-(aminomethyl)-1-{[3- (phenylmethanesulfon- amido)naphthalen-1-yl]- methyl}-1H-pyrrolo[2,3- b]pyridine-2-carboxylate | 598.0 | 96 | A |
| 19 | 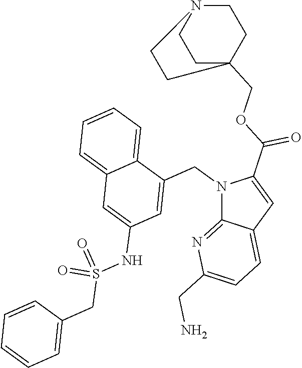 [SMILES: NCc1ccc2cc(C(=O)OCC34CCN(CC3)CC4)n(Cc3cc(NS(=O)(=O)Cc4ccccc4)cc4ccccc34)c2n1] | {1-azabicyclo[2.2.2]- octan-4-yl}methyl 6- (aminomethyl)-1-{[3- (phenylmethanesulfon- amido)naphthalen-1-yl]- methyl}-1H-pyrrolo[2,3- b]pyridine-2-carboxylate | 624.4 | 99 | A |
| 20 | 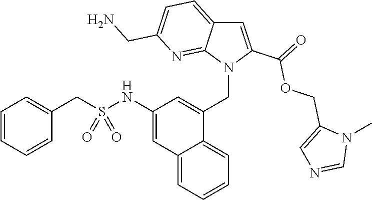 [SMILES: Cn1cncc1COC(=O)c1cc2ccc(CN)nc2n1Cc1cc(NS(=O)(=O)Cc2ccccc2)cc2ccccc12] | (1-methyl-1H-imidazol- 5-yl)methyl 6-(amino- methyl)-1-{[3-(phenyl- methanesulfonamido)- naphthalen-1-yl]methyl}- 1H-pyrrolo[2,3-b]pyr- idine-2-carboxylate | 595.0 | 100 | A |
| 21 | 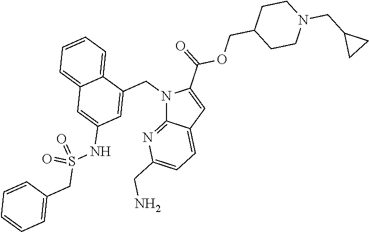 [SMILES: NCc1ccc2cc(C(=O)OCC3CCN(CC4CC4)CC3)n(Cc3cc(NS(=O)(=O)Cc4ccccc4)cc4ccccc34)c2n1] | [1-(cyclopropylmeth- yl)piperidin-4-yl]methyl 6-(aminomethyl)-1-{[3- (phenylmethanesulfon- amido)naphthalen-1-yl]- methyl}-1H-pyrrolo[2,3- b]pyridine-2-carboxylate | 652.0 | 100 | A |
| 22 | 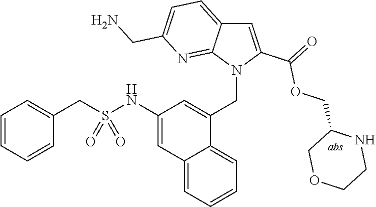 [SMILES: NCc1ccc2cc(C(=O)OC[C@H]3COCCN3)n(Cc3cc(NS(=O)(=O)Cc4ccccc4)cc4ccccc34)c2n1] | [(3R)-morpholin-3-yl]- methyl 6-(aminomethyl)- 1-{[3-(phenylmethane- sulfonamido)naphthalen- 1-yl]methyl}-1H-pyr- rolo[2,3-b]pyridine-2- carboxylate | 600.0 | 93 | A |
| 23 | 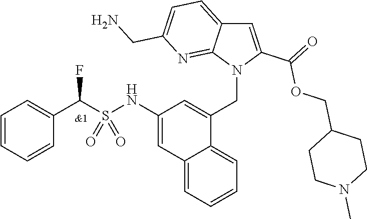 [SMILES: CN1CCC(COC(=O)c2cc3ccc(CN)nc3n2Cc2cc(NS(=O)(=O)[C@H](F)c3ccccc3)cc3ccccc23)CC1] | (1-methylpiperidin-4- yl)methyl 6-(amino- methyl)-1-({3-[fluoro- (phenyl)methanesulfon- amido]naphthalen-1-yl}- methyl)-1H-pyrrolo[2,3- b]pyridine-2-carboxylate | 630.4 | 95 | A |
| 24 | 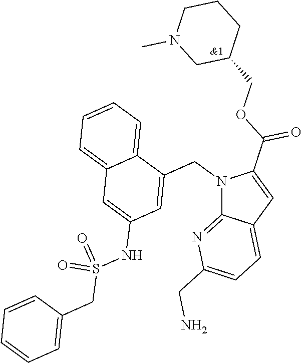 [SMILES: CN1CCC[C@H](COC(=O)c2cc3ccc(CN)nc3n2Cc2cc(NS(=O)(=O)Cc3ccccc3)cc3ccccc23)C1] | (1-methylpiperidin-3- yl)methyl 6-(amino- methyl)-1-{[3-(phenyl- methanesulfonamido)- naphthalen-1-yl]methyl}- 1H-pyrrolo[2,3-b]pyr- idine-2-carboxylate | 612.1 | 99 | A |
| 25 | 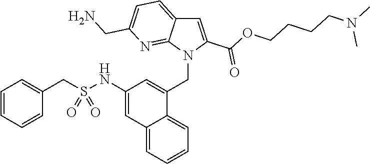 [SMILES: CN(C)CCCCOC(=O)c1cc2ccc(CN)nc2n1Cc1cc(NS(=O)(=O)Cc2ccccc2)cc2ccccc12] | 4-(dimethylamino)butyl 6-(aminomethyl)-1-{[3- (phenylmethanesulfon- amido)naphthalen-1-yl]- methyl}-1H-pyrrolo[2,3- b]pyridine-2-carboxylate | 600.4 | 99 | A |
| 26 | 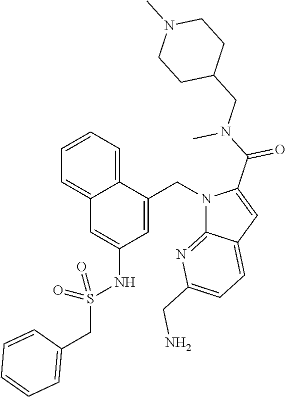 [SMILES: CN1CCC(CN(C)C(=O)c2cc3ccc(CN)nc3n2Cc2cc(NS(=O)(=O)Cc3ccccc3)cc3ccccc23)CC1] | 6-(aminomethyl)-N- methyl-N-[(1-methyl- piperidin-4-yl)methyl]- 1-{[3-(phenylmethane- sulfonamido)naphthalen- 1-yl]methyl}-1H-pyr- rolo[2,3-b]pyridine-2- carboxamide | 625.0 | 98 | A |
| 27 | 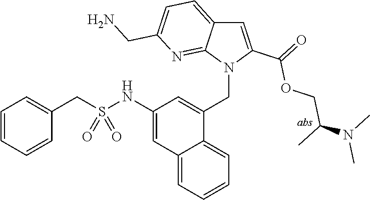 [SMILES: C[C@@H](COC(=O)c1cc2ccc(CN)nc2n1Cc1cc(NS(=O)(=O)Cc2ccccc2)cc2ccccc12)N(C)C] | (2S)-2-(dimethylamino)- propyl 6-(aminomethyl)- 1-{[3-(phenylmethane- sulfonamido)naphthalen- 1-yl]methyl}-1H-pyr- rolo[2,3-b]pyridine-2- carboxylate | 586.0 | 98 | A |
| 28 | 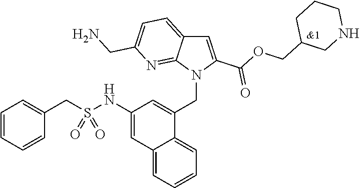 [SMILES: NCc1ccc2cc(C(=O)OCC3CCCNC3)n(Cc3cc(NS(=O)(=O)Cc4ccccc4)cc4ccccc34)c2n1] | (piperidin-3-yl)methyl 6-(aminomethyl)-1-{[3- (phenylmethanesulfon- amido)naphthalen-1-yl]- methyl}-1H-pyrrolo[2,3- b]pyridine-2-carboxylate | 598.2 | 99 | |
| 29 | 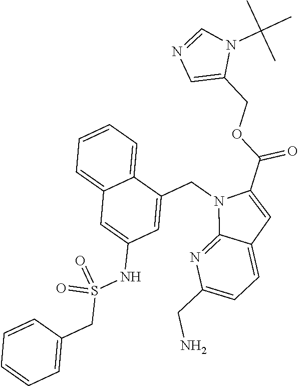 [SMILES: CC(C)(C)n1cncc1COC(=O)c1cc2ccc(CN)nc2n1Cc1cc(NS(=O)(=O)Cc2ccccc2)cc2ccccc12] | (1-tert-butyl-1H-imid- azol-5-yl)methyl 6- (aminomethyl)-1-{[3- (phenylmethanesulfon- amido)naphthalen-1-yl]- methyl}-1H-pyrrolo[2,3- b]pyridine-2-carboxylate | 637.2 | 95 | A |
| 30 | 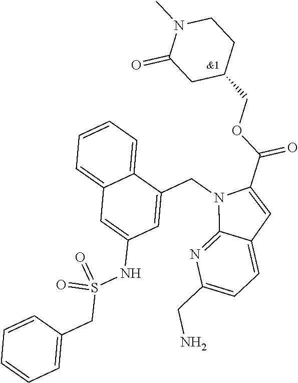 [SMILES: CN1CC[C@H](COC(=O)c2cc3ccc(CN)nc3n2Cc2cc(NS(=O)(=O)Cc3ccccc3)cc3ccccc23)CC1=O] | rac-[(4R)-1-methyl-2- oxopiperidin-4-yl]meth- yl 6-(aminomethyl)-1- {[3-(phenylmethane- sulfonamido)naphthalen- 1-yl]methyl}-1H-pyr- rolo[2,3-b]pyridine-2- carboxylate | 626.0 | 96 | A |
| 31 | 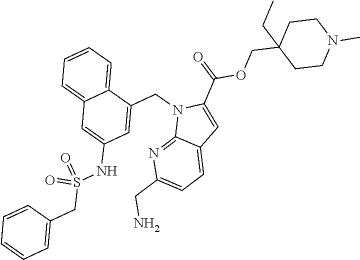 [SMILES: CCC1(COC(=O)c2cc3ccc(CN)nc3n2Cc2cc(NS(=O)(=O)Cc3ccccc3)cc3ccccc23)CCN(C)CC1] | (4-ethyl-1-methylpiper- idin-4-yl)methyl 6- (aminomethyl)-1-{[3- (phenylmethanesulfon- amido)naphthalen-1-yl]- methyl}-1H-pyrrolo[2,3- b]pyridine-2-carboxylate | 638.2 [M − H] | 97 | A |
| 32 | 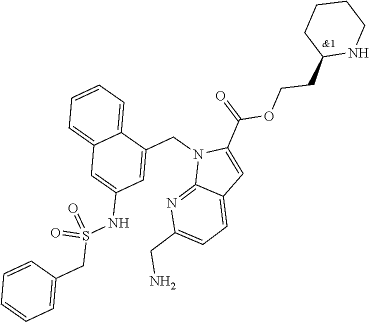 [SMILES: NCc1ccc2cc(C(=O)OCC[C@H]3CCCCN3)n(Cc3cc(NS(=O)(=O)Cc4ccccc4)cc4ccccc34)c2n1] | 2-(piperidin-2-yl)ethyl 6- (aminomethyl)-1-{[3- (phenylmethanesulfon- amido)naphthalen-1-yl]- methyl}-1H-pyrrolo[2,3- b]pyridine-2-carboxylate | 612.4 | 97 | A |
| 33 | 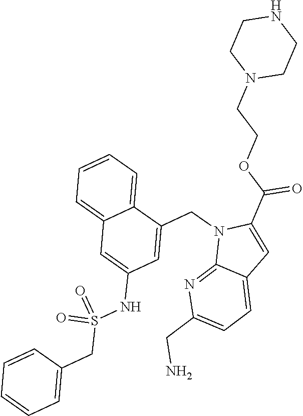 [SMILES: NCc1ccc2cc(C(=O)OCCN3CCNCC3)n(Cc3cc(NS(=O)(=O)Cc4ccccc4)cc4ccccc34)c2n1] | 2-(piperazin-1-yl)ethyl 6- (aminomethyl)-1-{[3- (phenylmethanesulfon- amido)naphthalen-1-yl]- methyl}-1H-pyrrolo[2,3- b]pyridine-2-carboxylate | 613.4 | 100 | A |
| 34 | 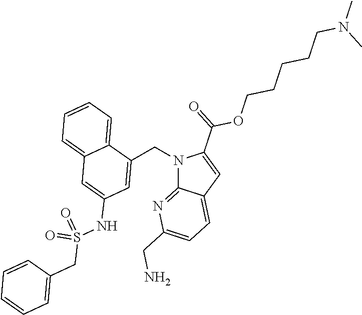 [SMILES: CN(C)CCCCCOC(=O)c1cc2ccc(CN)nc2n1Cc1cc(NS(=O)(=O)Cc2ccccc2)cc2ccccc12] | 5-(dimethylamino)pentyl 6-(aminomethyl)-1-{[3- (phenylmethanesulfon- amido)naphthalen-1-yl]- methyl}-1H-pyrrolo[2,3- b]pyridine-2-carboxylate | 614.2 | 99 | A |
| 35 | 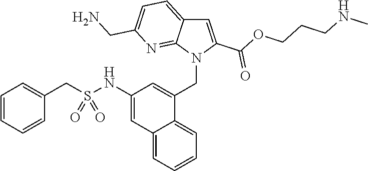 [SMILES: CNCCCOC(=O)c1cc2ccc(CN)nc2n1Cc1cc(NS(=O)(=O)Cc2ccccc2)cc2ccccc12] | 3-(methylamino)propyl 6-(aminomethyl)-1-{[3- (phenylmethanesulfon- amido)naphthalen-1-yl]- methyl}-1H-pyrrolo[2,3- b]pyridine-2-carboxylate | 572.4 | 100 | A |
| 36 | 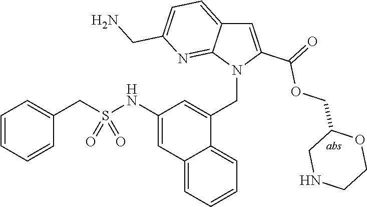 | [(2R)-morpholin-2-yl]- methyl6-(aminomethyl)- 1-{[3-(phenylmethane- sulfonamido)naphthalen- 1-yl]methyl}-1H-pyr- rolo[2,3-b]pyridine-2- carboxylate | 601.0 | 90 | A |
| 37 | 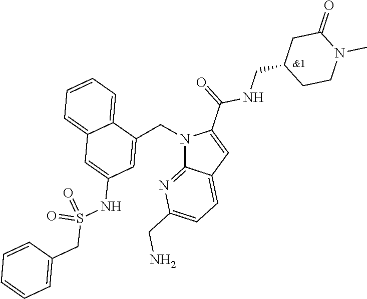 [SMILES: CN1CC[C@H](CNC(=O)c2cc3ccc(CN)nc3n2Cc2cc(NS(=O)(=O)Cc3ccccc3)cc3ccccc23)CC1=O] | rac-6-(aminomethyl)-N- {[(4R)-1-methyl-2-oxo- piperidin-4-yl]methyl}- 1-{[3-(phenylmethane- sulfonamido)naphthalen- 1-yl]methyl}-1H-pyr- rolo[2,3-b]pyridine-2- carboxamide | 625.0 | 99 | A |
| 38 | 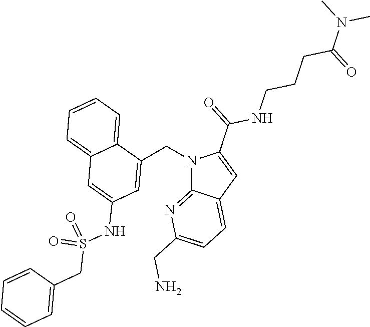 [SMILES: CN(C)C(=O)CCCNC(=O)c1cc2ccc(CN)nc2n1Cc1cc(NS(=O)(=O)Cc2ccccc2)cc2ccccc12] | 4-{[6-(aminomethyl)-1- {[3-(phenylmethane- sulfonamido)naphthalen- 1-yl]methyl}-1H-pyr- rolo[2,3-b]pyridin-2-yl]- formamido}-N,N- dimethylbutanamide | 613.0 | 100 | A |
| 39 | 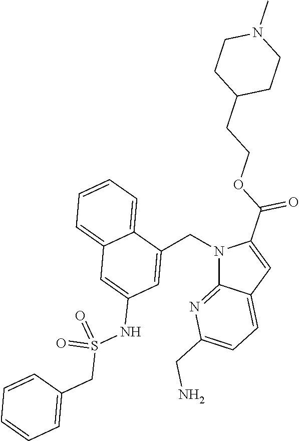 [SMILES: CN1CCC(CCOC(=O)c2cc3ccc(CN)nc3n2Cc2cc(NS(=O)(=O)Cc3ccccc3)cc3ccccc23)CC1] | 2-(1-methylpiperidin-4- yl)ethyl 6-(aminometh- yl)-1-{[3-(phenylmeth- anesulfonamido)naph- thalen-1-yl]methyl}-1H- pyrrolo[2,3-b]pyridine- 2-carboxylate | 626.4 | 99 | A |
| 40 | 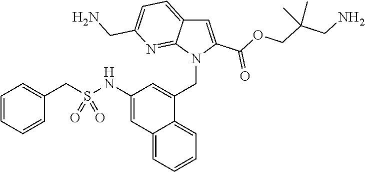 [SMILES: CC(C)(CN)COC(=O)c1cc2ccc(CN)nc2n1Cc1cc(NS(=O)(=O)Cc2ccccc2)cc2ccccc12] | 3-amino-2,2-dimethyl- propyl 6-(aminomethyl)- 1-{[3-(phenylmethane- sulfonamido)naphthalen- 1-yl]methyl}-1H-pyrrolo- [2,3-b]pyridine-2-car- boxylate | 586.0 | 100 | A |
| 41 | 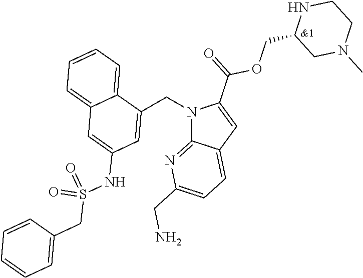 [SMILES: CN1CCN[C@@H](COC(=O)c2cc3ccc(CN)nc3n2Cc2cc(NS(=O)(=O)Cc3ccccc3)cc3ccccc23)C1] | (4-methylpiperazin-2- yl)methyl 6-(aminometh- yl)-1-{[3-(phenylmeth- ane-sulfonamido)naph- thalen-1-yl]methyl}-1H- pyrrolo[2,3-b]pyridine- 2-carboxylate | 613.4 | 99 | A |
| 42 | 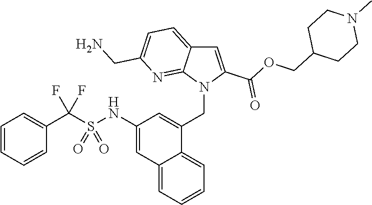 [SMILES: CN1CCC(COC(=O)c2cc3ccc(CN)nc3n2Cc2cc(NS(=O)(=O)C(F)(F)c3ccccc3)cc3ccccc23)CC1] | (1-methylpiperidin-4- yl)methyl 6-(aminometh- yl)-1-({3-[difluoro(phen- yl)methanesulfonamido]- naphthalen-1-yl}meth- yl)-1H-pyrrolo[2,3-b]- pyridine-2-carboxylate | 648.1 | 99 | A |
| 43 | 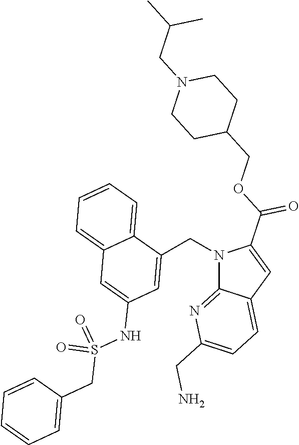 [SMILES: CC(C)CN1CCC(COC(=O)c2cc3ccc(CN)nc3n2Cc2cc(NS(=O)(=O)Cc3ccccc3)cc3ccccc23)CC1] | [1-(2-methylpropyl)- piperidin-4-yl]methyl 6- (aminomethyl)-1-{[3- (phenylmethanesulfon- amido)naphthalen-1-yl]- methyl}-1H-pyrrolo[2,3- b]pyridine-2-carboxylate | 654.0 | 100 | A |
| 44 | 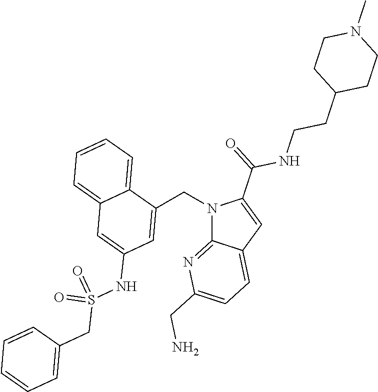 [SMILES: CN1CCC(CCNC(=O)c2cc3ccc(CN)nc3n2Cc2cc(NS(=O)(=O)Cc3ccccc3)cc3ccccc23)CC1] | 6-(aminomethyl)-N-[2- (1-methylpiperidin-4- yl)ethyl]-1-{[3-(phenyl- methanesulfonamido)- naphthalen-1-yl]methyl}- 1H-pyrrolo[2,3-b]pyr- idine-2-carboxamide | 625.4 | 99 | A |
| 45 | 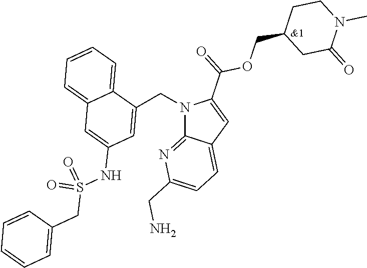 [SMILES: CN1CC[C@H](COC(=O)c2cc3ccc(CN)nc3n2Cc2cc(NS(=O)(=O)Cc3ccccc3)cc3ccccc23)CC1=O] | (1-methyl-2-oxopiper- idin-4-yl)methyl 6- (aminomethyl)-1-{[3- (phenylmethanesulfon- amido)naphthalen-1-yl]- methyl}-1H-pyrrolo[2,3- b]pyridine-2-carboxylate | 626.3 | 98 | A |
| 46 | 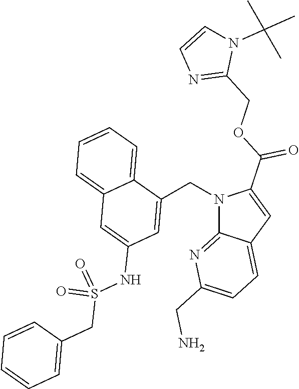 [SMILES: CC(C)(C)n1ccnc1COC(=O)c1cc2ccc(CN)nc2n1Cc1cc(NS(=O)(=O)Cc2ccccc2)cc2ccccc12] | (1-tert-butyl-1H-imid- azol-2-yl)methyl 6- (aminomethyl)-1-{[3- (phenylmethanesulfon- amido)naphthalen-1-yl]- methyl}-1H-pyrrolo[2,3- b]pyridine-2-carboxylate | 635.2 | 99 | A |
| 47 | 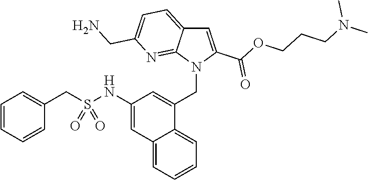 [SMILES: CN(C)CCCOC(=O)c1cc2ccc(CN)nc2n1Cc1cc(NS(=O)(=O)Cc2ccccc2)cc2ccccc12] | 3-(dimethylamino)prop- yl 6-(aminomethyl)-1- {[3-(phenylmethane- sulfonamido)naphthalen- 1-yl]methyl}-1H-pyr- rolo[2,3-b]pyridine-2- carboxylate | 586.4 | 100 | A |
| 48 | 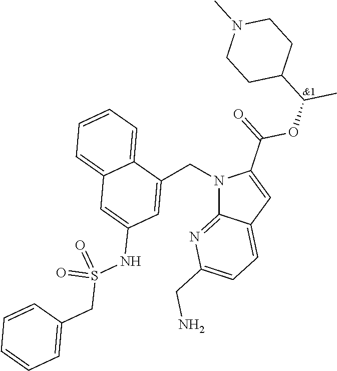 [SMILES: C[C@H](OC(=O)c1cc2ccc(CN)nc2n1Cc1cc(NS(=O)(=O)Cc2ccccc2)cc2ccccc12)C1CCN(C)CC1] | 1-(1-methylpiperidin-4- ylethyl 6-(aminomethyl)- 1-{[3-(phenylmethane- sulfonamido)naphthalen- 1-yl]methyl}-1H-pyr- rolo[2,3-b]pyridine-2- carboxylate | 626.4 | 96 | A |
| 49 | 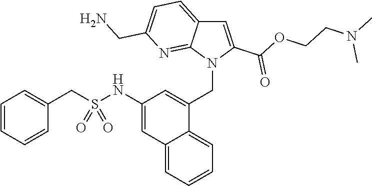 [SMILES: CN(C)CCOC(=O)c1cc2ccc(CN)nc2n1Cc1cc(NS(=O)(=O)Cc2ccccc2)cc2ccccc12] | 2-(dimethylamino)ethyl 6-(aminomethyl)-1-{[3- (phenylmethanesulfon- amido)naphthalen-1-yl]- methyl}-1H-pyrrolo[2,3- b]pyridine-2-carboxylate | 572.4 | 100 | A |
| 50 | 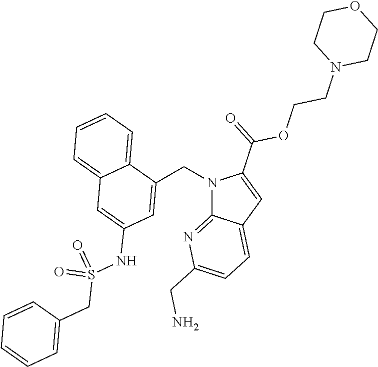 [SMILES: NCc1ccc2cc(C(=O)OCCN3CCOCC3)n(Cc3cc(NS(=O)(=O)Cc4ccccc4)cc4ccccc34)c2n1] | 2-(morpholin-4-yl)ethyl 6-(aminomethyl)-1-{[3- (phenylmethanesulfon- amido)naphthalen-1-yl]- methyl}-1H-pyrrolo[2,3- b]pyridine-2-carboxylate | 614.0 | 99 | A |
| 51 | 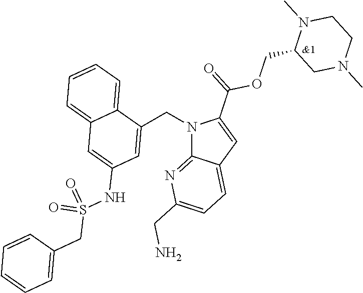 [SMILES: CN1CCN(C)[C@@H](COC(=O)c2cc3ccc(CN)nc3n2Cc2cc(NS(=O)(=O)Cc3ccccc3)cc3ccccc23)C1] | (1,4-dimethylpiperazin- 2-yl)methyl 6-(amino- methyl)-1-{[3-(phenyl- methanesulfonamido)- naphthalen-1-yl]methyl}- 1H-pyrrolo[2,3-b]pyr- idine-2-carboxylate | 627.5 | 99 | A |
| 52 | 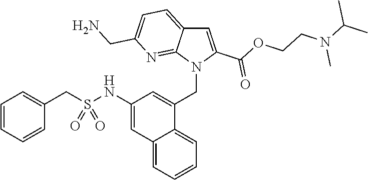 [SMILES: CC(C)N(C)CCOC(=O)c1cc2ccc(CN)nc2n1Cc1cc(NS(=O)(=O)Cc2ccccc2)cc2ccccc12] | 2-[methyl(propan-2-yl)- amino]ethyl 6-(amino- methyl)-1-{[3-(phenyl- methanesulfonamido)- naphthalen-1-yl]methyl}- 1H-pyrrolo[2,3-b]pyr- idine-2-carboxylate | 600.0 | 97 | A |
| 53 | 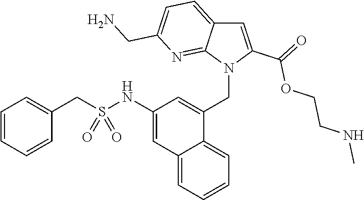 [SMILES: CNCCOC(=O)c1cc2ccc(CN)nc2n1Cc1cc(NS(=O)(=O)Cc2ccccc2)cc2ccccc12] | 2-(methylamino)ethyl 6- (aminomethyl)-1-{[3- (phenylmethanesulfon- amido)naphthalen-1-yl]- methyl}-1H-pyrrolo[2,3- b]pyridine-2-carboxylate | 558.3 | 100 | A |
| 54 | 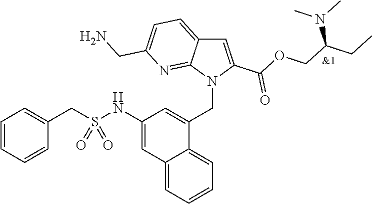 [SMILES: CC[C@@H](COC(=O)c1cc2ccc(CN)nc2n1Cc1cc(NS(=O)(=O)Cc2ccccc2)cc2ccccc12)N(C)C] | rac-(2R)-2-(dimethyl- amino)butyl 6-(amino- methyl)-1-{[3-(phenyl- methanesulfonamido)- naphthalen-1-yl]methyl}- 1H-pyrrolo[2,3-b]pyr- idine-2-carboxylate | 600.0 | 82 | A |
| 55 | 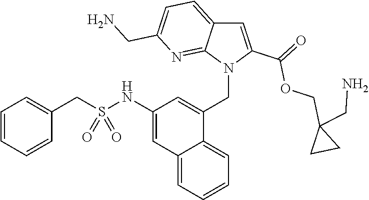 [SMILES: NCc1ccc2cc(C(=O)OCC3(CN)CC3)n(Cc3cc(NS(=O)(=O)Cc4ccccc4)cc4ccccc34)c2n1] | [1-(aminomethyl)cyclo- propyl]methyl 6-(amino- methyl)-1-{[3-(phenyl- methanesulfonamido)- naphthalen-1-yl]methyl}- 1H-pyrrolo[2,3-b]pyr- idine-2-carboxylate | 584.0 | 100 | A |
| 56 | 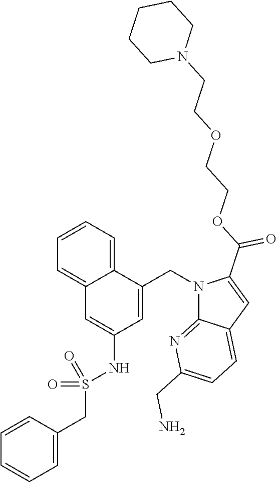 [SMILES: NCc1ccc2cc(C(=O)OCCOCCN3CCCCC3)n(Cc3cc(NS(=O)(=O)Cc4ccccc4)cc4ccccc34)c2n1] | 2-[2-(piperidin-1-yl)- ethoxy]ethyl 6-(amino- methyl)-1-{[3-(phenyl- methanesulfonamido)- naphthalen-1-yl]methyl}- 1H-pyrrolo[2,3-b]pyr- idine-2-carboxylate | 656.0 | 100 | A |
| 57 | 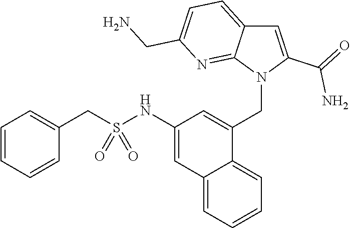 [SMILES: NCc1ccc2cc(C(N)=O)n(Cc3cc(NS(=O)(=O)Cc4ccccc4)cc4ccccc34)c2n1] | 6-(aminomethyl)-1-{[3- (phenylmethanesulfon- amido)naphthalen-1-yl]- methyl}-1H-pyrrolo[2,3- b]pyridine-2-carboxamide | 500.3 | 98 | A |
| 58 | 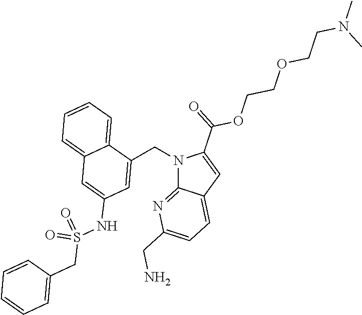 [SMILES: CN(C)CCOCCOC(=O)c1cc2ccc(CN)nc2n1Cc1cc(NS(=O)(=O)Cc2ccccc2)cc2ccccc12] | 2-[2-(dimethylamino)- ethoxy]ethyl 6-(amino- methyl)-1-{[3-(phenyl- methanesulfonamido)- naphthalen-1-yl]methyl}- 1H-pyrrolo[2,3-b]pyr- idine-2-carboxylate | 616.0 | 100 | A |
| 59 | 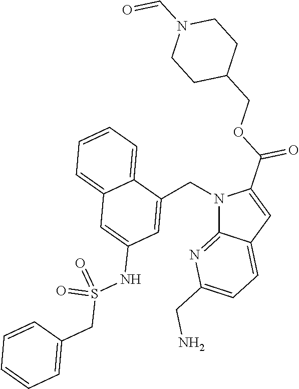 [SMILES: NCc1ccc2cc(C(=O)OCC3CCN(C=O)CC3)n(Cc3cc(NS(=O)(=O)Cc4ccccc4)cc4ccccc34)c2n1] | (1-formylpiperidin-4-yl)- methyl 6-(aminomethyl)- 1-{[3-(phenylmethane- sulfonamido)naphthalen- 1-yl]methyl}-1H-pyr- rolo[2,3-b]pyridine-2- carboxylate | 626.4 | 95 | A |
| 60 | 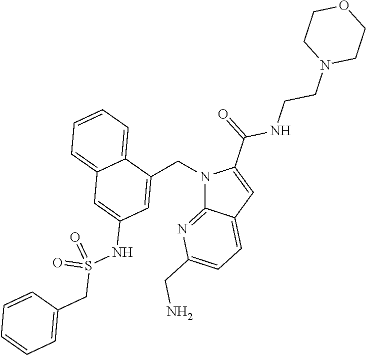 [SMILES: NCc1ccc2cc(C(=O)NCCN3CCOCC3)n(Cc3cc(NS(=O)(=O)Cc4ccccc4)cc4ccccc34)c2n1] | 6-(aminomethyl)-N-[2- (morpholin-4-yl)ethyl]- 1-{[3-(phenylmethane- sulfonamido)naphthalen- 1-yl]methyl}-1H-pyr- rolo[2,3-b]pyridine-2- carboxamide | 613.4 | 100 | A |
| 61 | 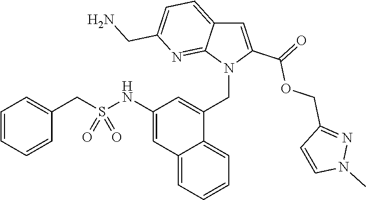 [SMILES: Cn1ccc(COC(=O)c2cc3ccc(CN)nc3n2Cc2cc(NS(=O)(=O)Cc3ccccc3)cc3ccccc23)n1] | (1-methyl-1H-pyrazol- 3-yl)methyl 6-(amino- methyl)-1-{[3-(phenyl- methanesulfonamido)- naphthalen-1-yl]methyl}- 1H-pyrrolo[2,3-b]pyr- idine-2-carboxylate | 595.3 | 98 | A |
| 62 | 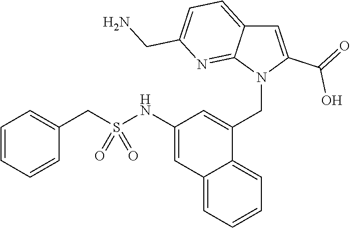 [SMILES: NCc1ccc2cc(C(=O)O)n(Cc3cc(NS(=O)(=O)Cc4ccccc4)cc4ccccc34)c2n1] | 6-(aminomethyl)-1-{[3- (phenylmethanesulfon- amido)naphthalen-1-yl]- methyl}-1H-pyrrolo[2,3- b]pyridine-2-carboxylic acid | 501.2 | 100 | A |
| 63 | 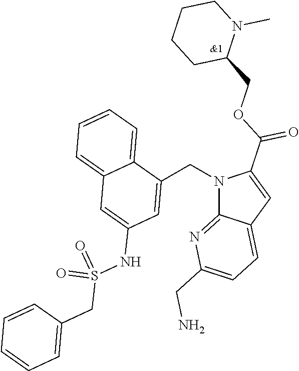 [SMILES: CN1CCCC[C@@H]1COC(=O)c1cc2ccc(CN)nc2n1Cc1cc(NS(=O)(=O)Cc2ccccc2)cc2ccccc12] | (1-methylpiperidin-2- yl)methyl 6-(aminometh- yl)-1-{[3-(phenylmeth- anesulfonamido)naph- thalen-1-yl]lmethyl}-1H- pyrrolo[2,3-b]pyridine- 2-carboxylate | 611.8 | 98 | A |
| 64 | 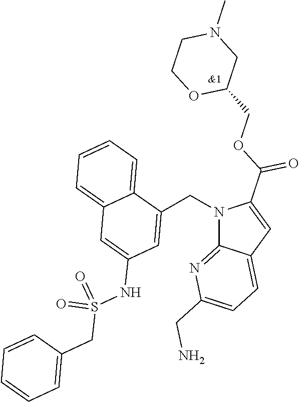 [SMILES: CN1CCO[C@@H](COC(=O)c2cc3ccc(CN)nc3n2Cc2cc(NS(=O)(=O)Cc3ccccc3)cc3ccccc23)C1] | (4-methylmorpholin-2- yl)methyl 6-(aminometh- yl)-1-{[3-(phenylmeth- anesulfonamido)naph- thalen-1-yl]methyl}-1H- pyrrolo[2,3-b]pyridine- 2-carboxylate | 613.7 | 98 | A |
| 65 | 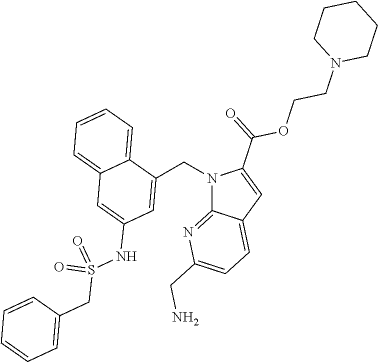 [SMILES: NCc1ccc2cc(C(=O)OCCN3CCCCC3)n(Cc3cc(NS(=O)(=O)Cc4ccccc4)cc4ccccc34)c2n1] | 2-(piperidin-1-yl)ethyl 6-(aminomethyl)-1-{[3- (phenylmethanesulfon- amido)naphthalen-1-yl]- methyl}-1H-pyrrolo[2,3- b]pyridine-2-carboxylate | 612.0 | 99 | A |
| 66 | 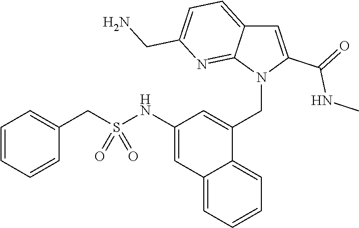 [SMILES: CNC(=O)c1cc2ccc(CN)nc2n1Cc1cc(NS(=O)(=O)Cc2ccccc2)cc2ccccc12] | 6-(aminomethyl)-N- methyl-1-(phenylmeth- anesulfonamido)naph- thalen-1-yl]methyl}-1H- pyrrolo[2,3-b]pyridine- 2-carboxamide | 514.3 | 99 | A |
| 67 | 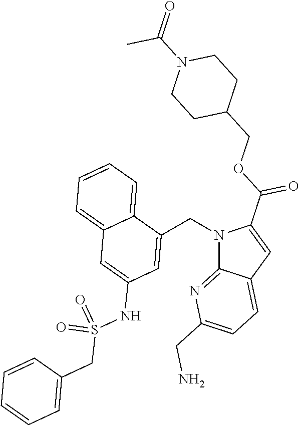 [SMILES: CC(=O)N1CCC(COC(=O)c2cc3ccc(CN)nc3n2Cc2cc(NS(=O)(=O)Cc3ccccc3)cc3ccccc23)CC1] | (1-acetylpiperidin-4-yl)- methyl 6-(aminomethyl)- 1-{[3-(phenylmethane- sulfonamido)naphthalen- 1-yl]methyl}-1H-pyr- rolo[2,3-b]pyridine-2- carboxylate | 640.0 | 99 | A |
| 68 | 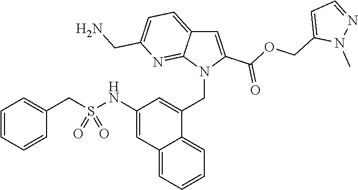 [SMILES: Cn1nccc1COC(=O)c1cc2ccc(CN)nc2n1Cc1cc(NS(=O)(=O)Cc2ccccc2)cc2ccccc12] | (1-methyl-1H-pyrazol- 5-yl)methyl 6-(amino- methyl)-1-{[3-(phenyl- methanesulfonamido)- naphthalen-1-yl]meth- yl}-1H-pyrrolo[2,3-b]- pyridine-2-carboxylate | 595.4 | 98 | A |
| 69 | 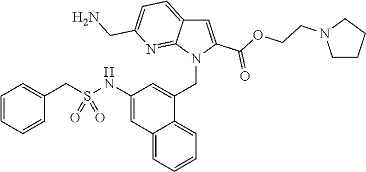 [SMILES: NCc1ccc2cc(C(=O)OCCN3CCCC3)n(Cc3cc(NS(=O)(=O)Cc4ccccc4)cc4ccccc34)c2n1] | 2-(pyrrolidin-1-yl)ethyl 6-(aminomethyl)-1-{[3- (phenylmethanesulfon- amido)naphthalen-1-yl]- methyl}-1H-pyrrolo[2,3- b]pyridine-2-carboxylate | 598.0 | 99 | A |
| 70 | 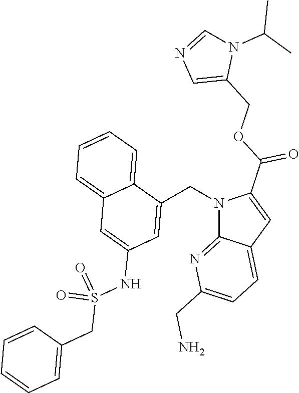 [SMILES: CC(C)n1cncc1COC(=O)c1cc2ccc(CN)nc2n1Cc1cc(NS(=O)(=O)Cc2ccccc2)cc2ccccc12] | [1-(propan-2-yl)-1H- imidazol-5-yl]methyl 6- (aminomethyl)-1-{[3- (phenylmethanesulfon- amido)naphthalen-1-yl]- methyl}-1H-pyrrolo[2,3- b]pyridine-2-carboxylate | 621.2 [M − H] | 97 | A |
| 71 | 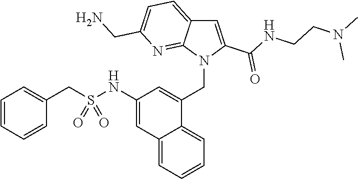 [SMILES: CN(C)CCNC(=O)c1cc2ccc(CN)nc2n1Cc1cc(NS(=O)(=O)Cc2ccccc2)cc2ccccc12] | 6-(aminomethyl)-N-[2- (dimethylamino)ethyl]-1- {[3-(phenylmethane- sulfonamido)naphthalen- 1-yl]methyl}-1H-pyr- rolo[2,3-b]pyridine-2- carboxamide | 571.3 | 100 | A |
| 72 | 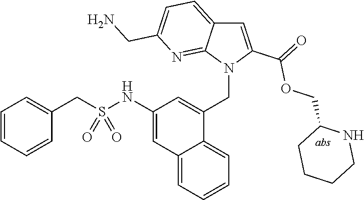 [SMILES: NCc1ccc2cc(C(=O)OC[C@H]3CCCCN3)n(Cc3cc(NS(=O)(=O)Cc4ccccc4)cc4ccccc34)c2n1] | [(2R)-piperidin-2-yl]- methyl 6-(aminomethyl)- 1-{[3-(phenylmethane- sulfonamido)naphthalen- 1-yl]methyl}-1H-pyr- rolo[2,3-b]pyridine-2- carboxylate | 598.0 | 97 | A |
| 73 | 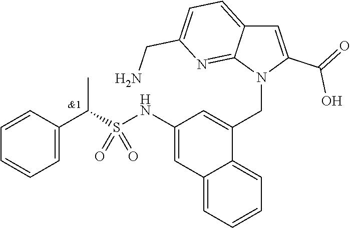 [SMILES: C[C@@H](c1ccccc1)S(=O)(=O)Nc1cc(Cn2c(C(=O)O)cc3ccc(CN)nc32)c2ccccc2c1] | 6-(aminomethyl)-1-{[3- (1-phenylethanesulfon- amido)naphthalen-1-yl]- methyl}-1H-pyrrolo[2,3- b]pyridine-2-carboxylic acid | 515.1 | 99 | A |
| 74 | 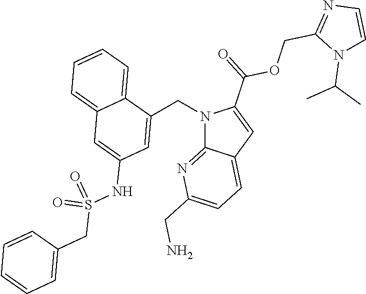 [SMILES: CC(C)n1ccnc1COC(=O)c1cc2ccc(CN)nc2n1Cc1cc(NS(=O)(=O)Cc2ccccc2)cc2ccccc12] | [1-(propan-2-yl)-1H- imidazol-2-yl]methyl 6- (aminomethyl)-1-{[3- (phenylmethanesulfon- amido)naphthalen-1-yl]- methyl}-1H-pyrrolo- [2,3-b]pyridine-2-car- boxylate | 621.2 [M − H] | 98 | A |
| 75 | 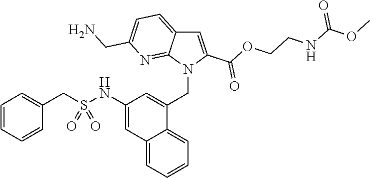 [SMILES: COC(=O)NCCOC(=O)c1cc2ccc(CN)nc2n1Cc1cc(NS(=O)(=O)Cc2ccccc2)cc2ccccc12] | 2-[(methoxycarbonyl)- amino]ethyl 6-(amino- methyl)-1-{[3-(phenyl- methanesulfonamido)- naphthalen-1-yl]methyl}- 1H-pyrrolo[2,3-b]pyr- idine-2-carboxylate | 602.1 | 99 | A |
| 76 | 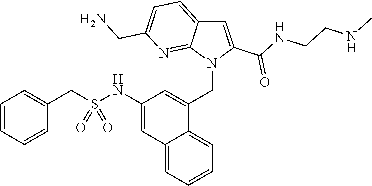 [SMILES: CNCCNC(=O)c1cc2ccc(CN)nc2n1Cc1cc(NS(=O)(=O)Cc2ccccc2)cc2ccccc12] | 6-(aminomethyl)-N-[2- (methylamino)ethyl]-1- {[3-(phenylmethane- sulfonamido)naphthalen- 1-yl]methyl}-1H-pyr- rolo[2,3-b]pyridine-2- carboxamide | 556.7 | 100 | A |
| 77 | 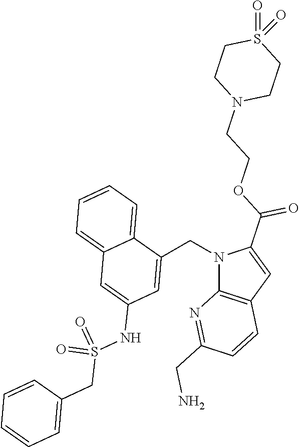 [SMILES: NCc1ccc2cc(C(=O)OCCN3CCS(=O)(=O)CC3)n(Cc3cc(NS(=O)(=O)Cc4ccccc4)cc4ccccc34)c2n1] | 2-(1,1-dioxo-1-thio- morpholin-4-yl)ethyl 6- (aminomethyl)-1-{[3- (phenylmethanesulfon- amido)naphthalen-1-yl]- methyl}-1H-pyrrolo[2,3- b]pyridine-2-carboxylate | 662.0 | 95 | A |
| 78 | 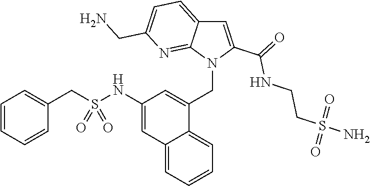 [SMILES: NCc1ccc2cc(C(=O)NCCS(N)(=O)=O)n(Cc3cc(NS(=O)(=O)Cc4ccccc4)cc4ccccc34)c2n1] | 6-(aminomethyl)-1-{[3- (phenylmethanesulfon- amido)naphthalen-1-yl]- methyl}-N-(2-sulfamoyl- ethyl)-1H-pyrrolo[2,3- b]pyridine-2-carboxamide | 607.0 | 100 | A |
| 79 | 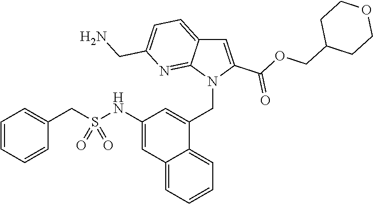 [SMILES: NCc1ccc2cc(C(=O)OCC3CCOCC3)n(Cc3cc(NS(=O)(=O)Cc4ccccc4)cc4ccccc34)c2n1] | (oxan-4-yl)methyl 6- (aminomethyl)-1-{[3- (phenylmethanesulfon- amido)naphthalen-1-yl]- methyl}-1H-pyrrolo[2,3- b]pyridine-2-carboxylate | 599.4 | 98 | A |
| 80 | 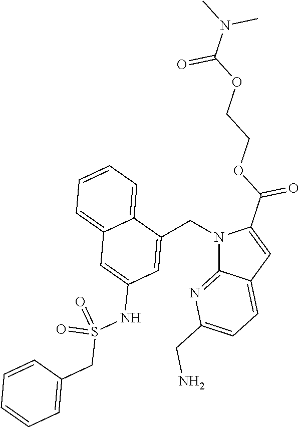 [SMILES: CN(C)C(=O)OCCOC(=O)c1cc2ccc(CN)nc2n1Cc1cc(NS(=O)(=O)Cc2ccccc2)cc2ccccc12] | 2-[(dimethylcarbamoyl)- oxy]ethyl 6-(aminometh- yl)-1-{[3-phenylmethane- sulfonamido)naphthalen- 1-yl]methyl}-1H-pyr- rolo[2,3-b]pyridine-2- carboxylate | 616.4 | 97 | A |
| 81 | 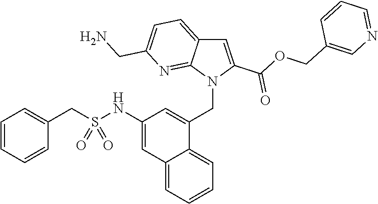 [SMILES: NCc1ccc2cc(C(=O)OCc3cccnc3)n(Cc3cc(NS(=O)(=O)Cc4ccccc4)cc4ccccc34)c2n1] | (pyridin-3-yl)methyl 6- (aminomethyl)-1-{[3- (phenylmethanesulfon- amido)naphthalen-1-yl]- methyl}-1H-pyrrolo[2,3- b]pyridine-2-carboxylate | 592.3 | 99 | A |
| 82 | 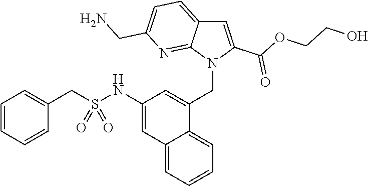 [SMILES: NCc1ccc2cc(C(=O)OCCO)n(Cc3cc(NS(=O)(=O)Cc4ccccc4)cc4ccccc34)c2n1] | 2-hydroxyethyl 6-(amino- methyl)-1-{[3-(phenyl- methanesulfonamido)- naphthalen-1-yl]methyl}- 1H-pyrrolo[2,3-b]pyr- idine-2-carboxylate | 545.1 | 100 | B |
| 83 | 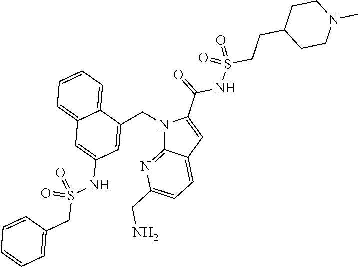 [SMILES: CN1CCC(CCS(=O)(=O)NC(=O)c2cc3ccc(CN)nc3n2Cc2cc(NS(=O)(=O)Cc3ccccc3)cc3ccccc23)CC1] | 6-(aminomethyl)-N-[2- (1-methylpiperidin-4-yl)- ethanesulfonyl]-1-{[3- (phenylmethanesulfon- amido)naphthalen-1-yl]- methyl}-1H-pyrrolo[2,3- b]pyridine-2-carboxamide | 689.3 | 100 | B |
| 84 | 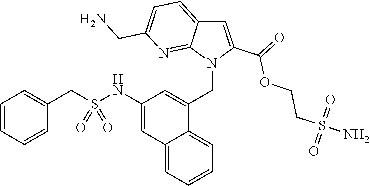 [SMILES: NCc1ccc2cc(C(=O)OCCS(N)(=O)=O)n(Cc3cc(NS(=O)(=O)Cc4ccccc4)cc4ccccc34)c2n1] | 2-sulfamoylethyl 6- (aminomethyl)-1-{[3- (phenylmethanesulfon- amido)naphthalen-1-yl]- methyl}-1H-pyrrolo[2,3- b]pyridine-2-carboxylate | 608.0 | 100 | B |
| 85 | 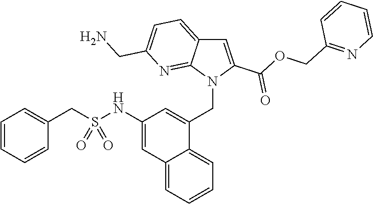 [SMILES: NCc1ccc2cc(C(=O)OCc3ccccn3)n(Cc3cc(NS(=O)(=O)Cc4ccccc4)cc4ccccc34)c2n1] | (pyridin-2-yl)methyl 6- (aminomethyl)-1-{[3- (phenylmethanesulfon- amido)naphthalen-1-yl]- methyl}-1H-pyrrolo[2,3- b]pyridine-2-carboxylate | 592.2 | 97 | B |
| 86 | 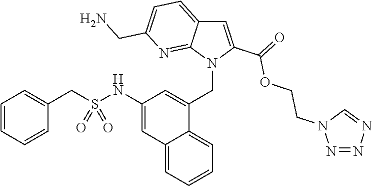 [SMILES: NCc1ccc2cc(C(=O)OCCn3cnnn3)n(Cc3cc(NS(=O)(=O)Cc4ccccc4)cc4ccccc34)c2n1] | 2-(1H-1,2,3,4-tetrazol- 1-yl)ethyl 6-(amino- methyl)-1-{[3-(phenyl- methanesulfonamido)- naphthalen-1-yl]methyl}- 1H-pyrrolo[2,3-b]pyr- idine-2-carboxylate | 597.3 | 99 | B |
| 87 | 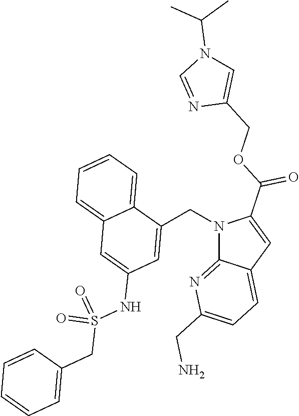 [SMILES: CC(C)n1cnc(COC(=O)c2cc3ccc(CN)nc3n2Cc2cc(NS(=O)(=O)Cc3ccccc3)cc3ccccc23)c1] | [1-(propan-2-yl)-1H- imidazol-4-yl]methyl 6- (aminomethyl)-1-{[3- (phenylmethanesulfon- amido)naphthalen-1-yl]- methyl}-1H-pyrrolo[2,3- b]pyridine-2-carboxylate | 621.2 [M − H] | 96 | B |
| 88 | 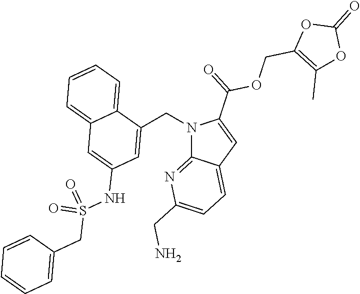 [SMILES: Cc1oc(=O)oc1COC(=O)c1cc2ccc(CN)nc2n1Cc1cc(NS(=O)(=O)Cc2ccccc2)cc2ccccc12] | (5-methyl-2-oxo-2H-1,3- dioxol-4-yl)methyl 6- (aminomethyl)-1-{[3- (phenylmethanesulfon- amido)naphthalen-1-yl]- methyl}-1H-pyrrolo[2,3- b]pyridine-2-carboxylate | 613.3 | 100 | B |
| 89 | 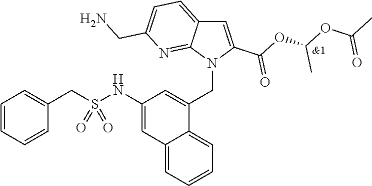 [SMILES: CC(=O)O[C@H](C)OC(=O)c1cc2ccc(CN)nc2n1Cc1cc(NS(=O)(=O)Cc2ccccc2)cc2ccccc12] | 1-[6-(aminomethyl)-1- {[3-(phenylmethanesul- fonamido)naphthalen-1- yl]methyl}-1H-pyrrolo- [2,3-b]pyridine-2-carbon- yloxy]ethyl acetate | 587.2 | 90 | B |
| 90 | 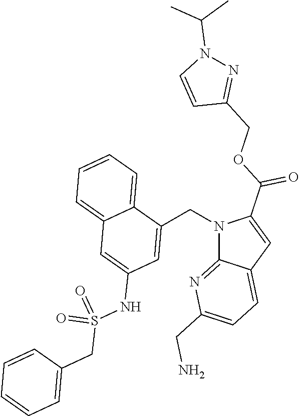 [SMILES: CC(C)n1ccc(COC(=O)c2cc3ccc(CN)nc3n2Cc2cc(NS(=O)(=O)Cc3ccccc3)cc3ccccc23)n1] | [1-(propan-2-yl)-1H- pyrazol-3-yl]methyl 6- (aminomethyl)-1-{[3- (phenylmethanesulfon- amido)naphthalen-1-yl]- methyl}-1H-pyrrolo[2,3- b]pyridine-2-carboxylate | 623.3 | 98 | B |
| 91 | 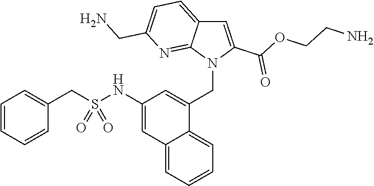 [SMILES: NCCOC(=O)c1cc2ccc(CN)nc2n1Cc1cc(NS(=O)(=O)Cc2ccccc2)cc2ccccc12] | 2-aminoethyl 6-(amino- methyl)-1-{[3-(phenyl- methanesulfonamido) naphthalen-1-yl]methyl}- 1H-pyrrolo[2,3-b]pyr- idine-2-carboxylate | 544.3 | 99 | B |
| 92 | 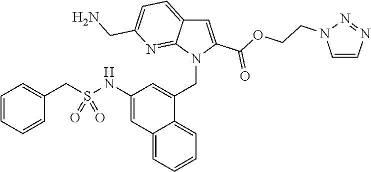 [SMILES: NCc1ccc2cc(C(=O)OCCn3ccnn3)n(Cc3cc(NS(=O)(=O)Cc4ccccc4)cc4ccccc34)c2n1] | 2-(1H-1,2,3-triazol-1-yl- ethyl 6-(aminomethyl)- 1-{[3-(phenylmethane- sulfonamido)naphthalen- 1-yl]methyl}-1H-pyrrolo- [2,3-b]pyridine-2-carbox- ylate | 596.3 | 92 | B |
| 93 | 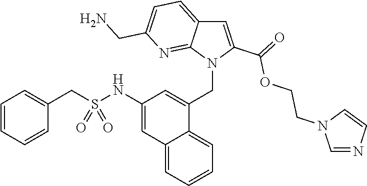 [SMILES: NCc1ccc2cc(C(=O)OCCn3ccnc3)n(Cc3cc(NS(=O)(=O)Cc4ccccc4)cc4ccccc34)c2n1] | 2-(1H-imidazol-1-yl)- ethyl 6-(aminomethyl)-1- {[3-(phenylmethanesul- fonamido)naphthalen-1- yl]methyl}-1H-pyrrolo- [2,3-b]pyridine-2-car- boxylate | 298.0 | 95 | B |
| 94 | 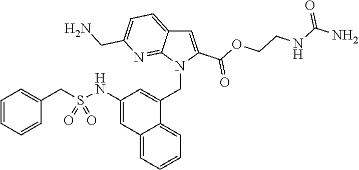 [SMILES: NCc1ccc2cc(C(=O)OCCNC(N)=O)n(Cc3cc(NS(=O)(=O)Cc4ccccc4)cc4ccccc34)c2n1] | 2-(carbamoylamino)- ethyl 6-(aminomethyl)-1- {[3-(phenylmethanesul- fonamido)naphthalen-1- yl]methyl}-1H-pyrrolo- [2,3-b]pyridine-2-carbox- ylate | 587.0 | 99 | B |
| 95 | 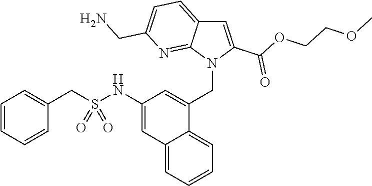 [SMILES: COCCOC(=O)c1cc2ccc(CN)nc2n1Cc1cc(NS(=O)(=O)Cc2ccccc2)cc2ccccc12] | 2-methoxyethyl 6- (aminomethyl)-1-{[3- (phenylmethanesulfon- amido)naphthalen-1-yl]- methyl}-1H-pyrrolo[2,3- b]pyridine-2-carboxylate | 559.3 | 99 | B |
| 96 | 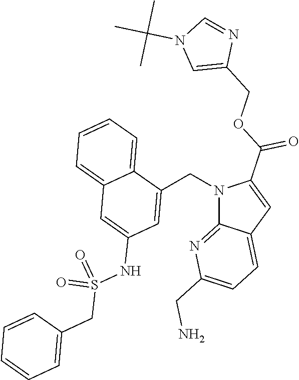 [SMILES: CC(C)(C)n1cnc(COC(=O)c2cc3ccc(CN)nc3n2Cc2cc(NS(=O)(=O)Cc3ccccc3)cc3ccccc23)c1] | (1-tert-butyl-1H-imid- azol-4-yl)methyl 6- (aminomethyl)-1-{[3- (phenylmethanesulfon- amido)naphthalen-1-yl]- methyl}-1H-pyrrolo[2,3- b]pyridine-2-carboxylate | 636.8 | 99 | B |
| 97 | 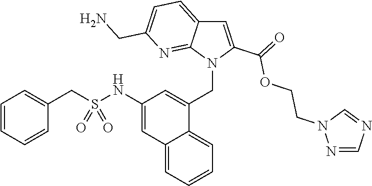 [SMILES: NCc1ccc2cc(C(=O)OCCn3cncn3)n(Cc3cc(NS(=O)(=O)Cc4ccccc4)cc4ccccc34)c2n1] | 2-(1H-1,2,4-triazol-1- yl)ethyl 6-(aminometh- yl)-1-{[3-phenylmeth- ane-sulfonamido)naph- thalen-1-yl]methyl}-1H- pyrrolo-[2,3-b]pyridine- 2-carboxylate | 596.3 | 100 | B |
| 98 | 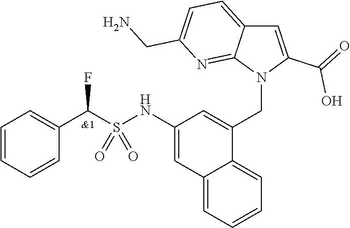 [SMILES: NCc1ccc2cc(C(=O)O)n(Cc3cc(NS(=O)(=O)[C@H](F)c4ccccc4)cc4ccccc34)c2n1] | 6-(aminomethyl)-1-({3- [fluoro(phenyl)methane- sulfonamido]naphthalen- 1-yl}methyl)-1H-pyr- rolo[2,3-b]pyridine-2- carboxylic acid | 519.3 | 99 | B |
| 99 | 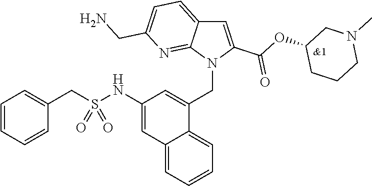 [SMILES: CN1CCC[C@H](OC(=O)c2cc3ccc(CN)nc3n2Cc2cc(NS(=O)(=O)Cc3ccccc3)cc3ccccc23)C1] | 1-methylpiperidin-3-yl 6-(aminomethyl)-1-{[3- (phenylmethanesulfon- amido)naphthalen-1-yl]- methyl}-1H-pyrrolo[2,3- b]pyridine-2-carboxylate | 597.7 | 100 | B |
| 100 | 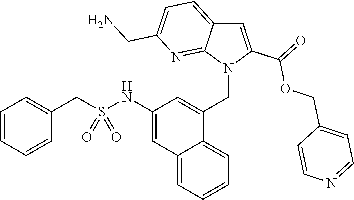 [SMILES: NCc1ccc2cc(C(=O)OCc3ccncc3)n(Cc3cc(NS(=O)(=O)Cc4ccccc4)cc4ccccc34)c2n1] | (pyridin-4-yl)methyl 6- (aminomethyl)-1-{[3- (phenylmethanesulfon- amido)naphthalen-1-yl]- methyl}-1H-pyrrolo[2,3- b]pyridine-2-carboxylate | 592.1 | 97 | B |
| 101 | 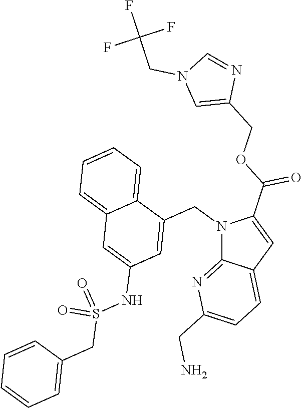 [SMILES: NCc1ccc2cc(C(=O)OCc3cn(CC(F)(F)F)cn3)n(Cc3cc(NS(=O)(=O)Cc4ccccc4)cc4ccccc34)c2n1] | [1-(2,2,2-trifluoroethyl)- 1H-imidazol-4-yl]methyl 6-(aminomethyl)-1-{[3- (phenylmethanesulfon- amido)naphthalen-1-yl]- methyl}-1H-pyrrolo[2,3- b]pyridine-2-carboxylate | 662.9 | 96 | B |
| 102 | 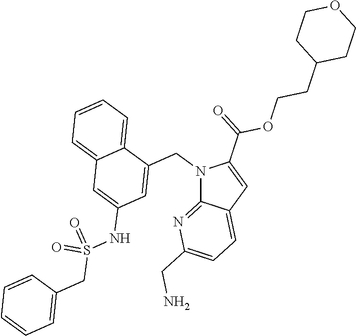 [SMILES: NCc1ccc2cc(C(=O)OCCC3CCOCC3)n(Cc3cc(NS(=O)(=O)Cc4ccccc4)cc4ccccc34)c2n1] | 2-(oxan-4-yl)ethyl 6- (aminomethyl)-1-{[3- (phenylmethanesulfon- amido)naphthalen-1-yl]- methyl}-1H-pyrrolo[2,3- b]pyridine-2-carboxylate | 611.2 [M − H] | 100 | B |
| 103 | 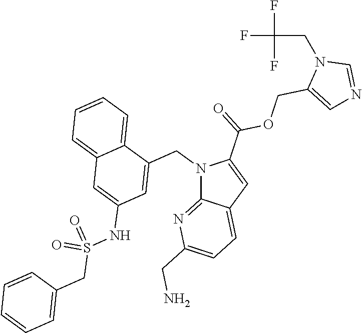 [SMILES: NCc1ccc2cc(C(=O)OCc3cncn3CC(F)(F)F)n(Cc3cc(NS(=O)(=O)Cc4ccccc4)cc4ccccc34)c2n1] | [1-(2,2,2-trifluoroethyl)- 1H-imidazol-5-yl]methyl 6-(aminomethyl)-1-{[3- (phenylmethanesulfon- amido)naphthalen-1-yl]- methyl}-1H-pyrrolo[2,3- b]pyridine-2-carboxylate | 662.7 | 99 | B |
| 104 | 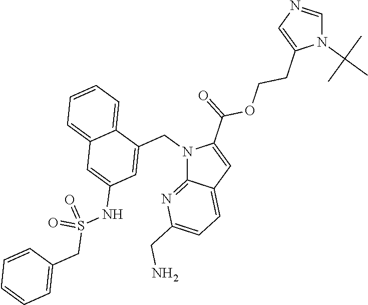 [SMILES: CC(C)(C)n1cncc1CCOC(=O)c1cc2ccc(CN)nc2n1Cc1cc(NS(=O)(=O)Cc2ccccc2)cc2ccccc12] | 2-(1-tert-butyl-1H-imid- azol-5-yl)ethyl 6-(amino- methyl)-1-{[3-(phenyl- methanesulfonamido)- naphthalen-1-yl]methyl}- 1H-pyrrolo[2,3-b]pyr- idine-2-carboxylate | 651.1 | 98 | B |
| 105 | 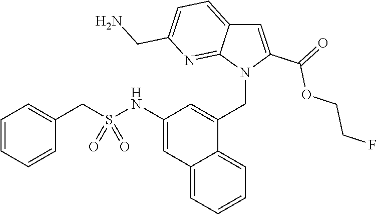 [SMILES: NCc1ccc2cc(C(=O)OCCF)n(Cc3cc(NS(=O)(=O)Cc4ccccc4)cc4ccccc34)c2n1] | 2-fluoroethyl 6-(amino- methyl)-1-{[3-(phenyl- methanesulfonamido)- naphthalen-1-yl]methyl}- 1H-pyrrolo[2,3-b]pyr- idine-2-carboxylate | 546.6 | 100 | B |
| 106 | 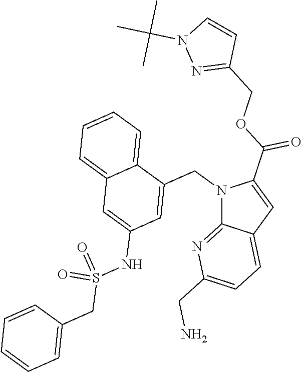 [SMILES: CC(C)(C)n1ccc(COC(=O)c2cc3ccc(CN)nc3n2Cc2cc(NS(=O)(=O)Cc3ccccc3)cc3ccccc23)n1] | (1-tert-butyl-1H-pyrazol- 3-yl)methyl 6-(amino- methyl)-1-{[3-(phenyl- methanesulfonamido)- naphthalen-1-yl]methyl}- 1H-pyrrolo[2,3-b]pyr- idine-2-carboxylate | 637.0 | 99 | B |
| 107 | 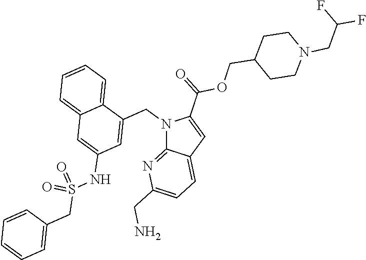 [SMILES: NCc1ccc2cc(C(=O)OCC3CCN(CC(F)F)CC3)n(Cc3cc(NS(=O)(=O)Cc4ccccc4)cc4ccccc34)c2n1] | [1-(2,2-difluoroethyl)- piperidin-4-yl]methyl 6- (aminomethyl)-1-{[3- (phenylmethanesulfon- amido)naphthalen-1-yl]- methyl}-1H-pyrrolo[2,3- b]pyridine-2-carboxylate | 662.0 | 99 | B |
| 108 | 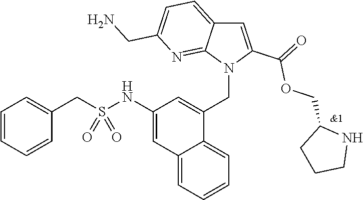 [SMILES: NCc1ccc2cc(C(=O)OC[C@H]3CCCN3)n(Cc3cc(NS(=O)(=O)Cc4ccccc4)cc4ccccc34)c2n1] | rac-[(2R)-pyrrolidin-2- yl]methyl 6-(aminometh- yl)-1-{[3-(phenylmeth- anesulfonamido)naph- thalen-1-yl]methyl}-1H- pyrrolo[2,3-b]pyridine- 2-carboxylate | 584.0 | 87 | B |
| 109 | 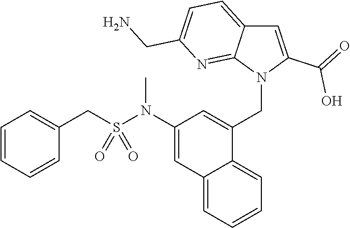 [SMILES: CN(c1cc(Cn2c(C(=O)O)cc3ccc(CN)nc32)c2ccccc2c1)S(=O)(=O)Cc1ccccc1] | 6-(aminomethyl)-1-{[3- (N-methylphenylmeth- anesulfonamido)naph- thalen-1-yl]methyl}-1H- pyrrolo[2,3-b]pyridine- 2-carboxylic acid | 515.3 | 99 | B |
| 110 | 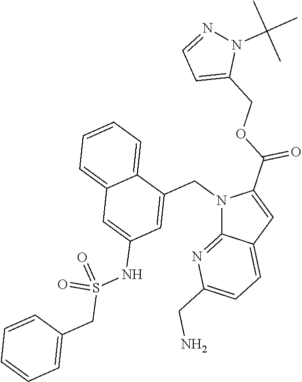 [SMILES: CC(C)(C)n1nccc1COC(=O)c1cc2ccc(CN)nc2n1Cc1cc(NS(=O)(=O)Cc2ccccc2)cc2ccccc12] | (1-tert-butyl-1H-pyrazol- 5-yl)methyl 6-(amino- methyl)-1-(phenylmeth- anesulfonamido)naph- thalen-1-yl]methyl}-1H- pyrrolo[2,3-b]pyridine- 2-carboxylate | 637.2 | 100 | B |
| 111 | 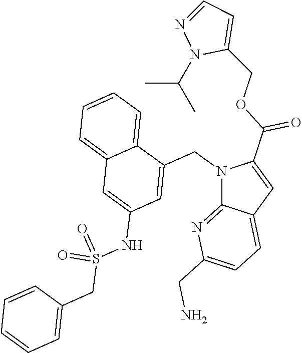 [SMILES: CC(C)n1nccc1COC(=O)c1cc2ccc(CN)nc2n1Cc1cc(NS(=O)(=O)Cc2ccccc2)cc2ccccc12] | [1-(propan-2-yl)-1H- pyrazol-5-yl]methyl 6- (aminomethyl)-1-{[3- (phenylmethanesulfon- amido)naphthalen-1-yl]- methyl}-1H-pyrrolo- [2,3-b]pyridine-2-car- boxylate | 623.0 | 96 | B |
| 112 | 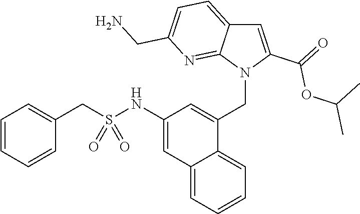 [SMILES: CC(C)OC(=O)c1cc2ccc(CN)nc2n1Cc1cc(NS(=O)(=O)Cc2ccccc2)cc2ccccc12] | propan-2-yl 6-(amino- methyl)-1-{[3-(phenyl- methanesulfonamido)- naphthalen-1-yl]methyl}- 1H-pyrrolo[2,3-b]pyr- idine-2-carboxylate | 543.3 | 99 | C |
| 113 | 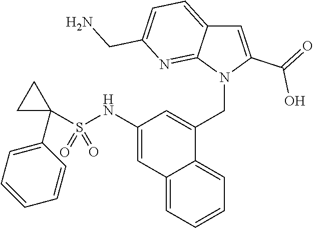 [SMILES: NCc1ccc2cc(C(=O)O)n(Cc3cc(NS(=O)(=O)C4(c5ccccc5)CC4)cc4ccccc34)c2n1] | 6-(aminomethyl)-1-{[3- (1-phenylcyclopropane- sulfonamido)naphthalen- 1-yl]methyl}-1H-pyr- rolo[2,3-b]pyridine-2- carboxylic acid | 527.3 | 99 | C |
| 114 | 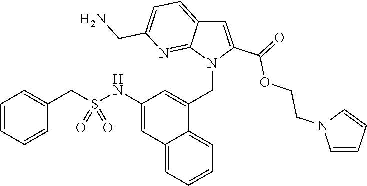 [SMILES: NCc1ccc2cc(C(=O)OCCn3cccc3)n(Cc3cc(NS(=O)(=O)Cc4ccccc4)cc4ccccc34)c2n1] | 2-(1H-pyrrol-1-yl)ethyl 6-(aminomethyl)-1-{[3- (phenylmethanesulfon- amido)naphthalen-1-yl]- methyl}-1H-pyrrolo[2,3- b]pyridine-2-carboxylate | 554.0 | 88 | C |
| 115 | 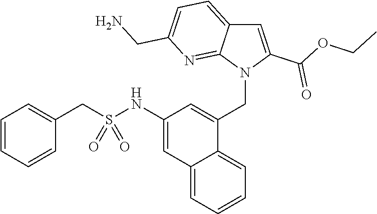 [SMILES: CCOC(=O)c1cc2ccc(CN)nc2n1Cc1cc(NS(=O)(=O)Cc2ccccc2)cc2ccccc12] | ethyl 6-(aminomethyl)-1- {[3-(phenylmethanesul- fonamido)naphthalen-1- yl]methyl}-1H-pyrrolo- [2,3-b]pyridine-2-car- boxylate | 529.1 | 99 | C |
| 116 | 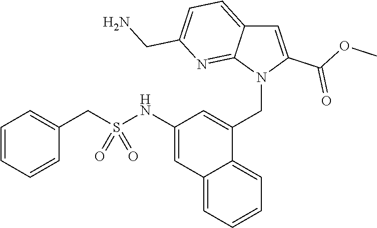 [SMILES: COC(=O)c1cc2ccc(CN)nc2n1Cc1cc(NS(=O)(=O)Cc2ccccc2)cc2ccccc12] | methyl 6-(aminomethyl)- 1-{[3-(phenylmethane- sulfonamido)naphthalen- 1-yl]methyl}-1H-pyrrolo- [2,3-b]pyridine-2-car- boxylate | 515.3 | 99 | C |
| 117 | 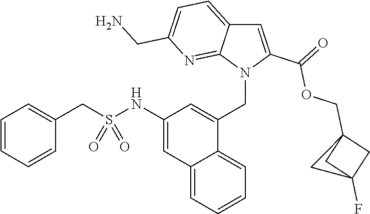 [SMILES: NCc1ccc2cc(C(=O)OCC34CC(F)(C3)C4)n(Cc3cc(NS(=O)(=O)Cc4ccccc4)cc4ccccc34)c2n1] | {3-fluorobicyclo[1.1.1]- pentan-1-yl}methyl 6- (aminomethyl)-1-{[3- (phenylmethanesulfon- amido)naphthalen-1-yl]- methyl}-1H-pyrrolo[2,3- b]pyridine-2-carboxylate | 599.3 | 100 | C |
| 118 | 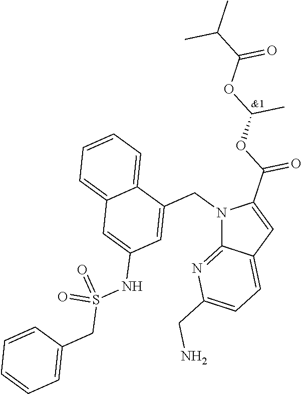 [SMILES: CC(C)C(=O)O[C@H](C)OC(=O)c1cc2ccc(CN)nc2n1Cc1cc(NS(=O)(=O)Cc2ccccc2)cc2ccccc12] | 1-[6-(aminomethyl)-1- {[3-(phenylmethanesul- fonamido)naphthalen-1- yl]methyl}-1H-pyrrolo- [2,3-b]pyridine-2-car- bonyloxylethyl 2-meth- ylpropanoate | 615.2 | 93 | C |
| 119 | 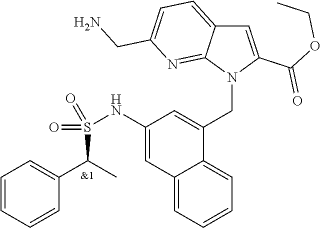 [SMILES: CCOC(=O)c1cc2ccc(CN)nc2n1Cc1cc(NS(=O)(=O)[C@@H](C)c2ccccc2)cc2ccccc12] | ethyl 6-(aminomethyl)-1- {[3-(1-phenylethanesul- fonamido)naphthalen-1- yl]methyl}-1H-pyrrolo- [2,3-b]pyridine-2-car- boxylate | 543.4 | 96 | C |
| 120 | 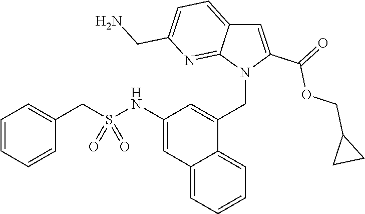 [SMILES: NCc1ccc2cc(C(=O)OCC3CC3)n(Cc3cc(NS(=O)(=O)Cc4ccccc4)cc4ccccc34)c2n1] | cyclopropylmethyl 6- (aminomethyl)-1-{[3- (phenylmethanesulfon- amido)naphthalen-1-yl]- methyl}-1H-pyrrolo[2,3- b]pyridine-2-carboxylate | 555.4 | 99 | C |
| 121 | 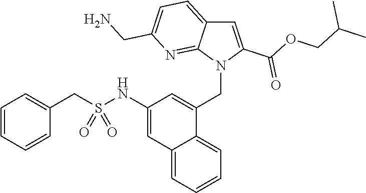 [SMILES: CC(C)COC(=O)c1cc2ccc(CN)nc2n1Cc1cc(NS(=O)(=O)Cc2ccccc2)cc2ccccc12] | 2-methylpropyl 6- (aminomethyl)-1-{[3- (phenylmethanesulfon- amido)naphthalen-1-yl]- methyl}-1H-pyrrolo[2,3- b]pyridine-2-carboxylate | 557.4 | 100 | C |
| 122 | 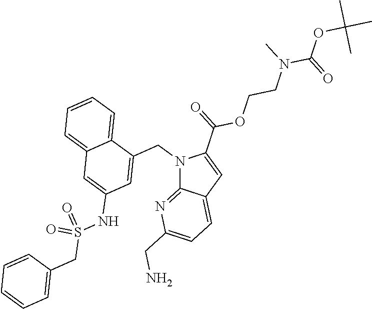 [SMILES: CN(CCOC(=O)c1cc2ccc(CN)nc2n1Cc1cc(NS(=O)(=O)Cc2ccccc2)cc2ccccc12)C(=O)OC(C)(C)C] | 2-{[(tert-butoxy)carbon- yl](methyl)amino}ethyl 6-(aminomethyl)-1-{[3- (phenylmethanesulfon- amido)naphthalen-1-yl]- methyl}-1H-pyrrolo[2,3- b]pyridine-2-carboxylate | 658.4 | 95 | C |
| 123 | 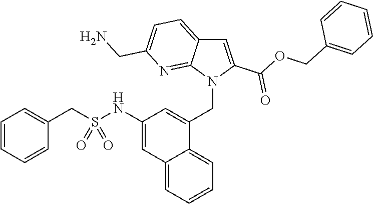 [SMILES: NCc1ccc2cc(C(=O)OCc3ccccc3)n(Cc3cc(NS(=O)(=O)Cc4ccccc4)cc4ccccc34)c2n1] | benzyl 6-(aminomethyl)- 1-{[3-(phenylmethane- sulfonamido)naphthalen- 1-yl]methyl}-1H-pyr- rolo[2,3-b]pyridine-2- carboxylate | 590.7 | 99 | C |
| 124 | 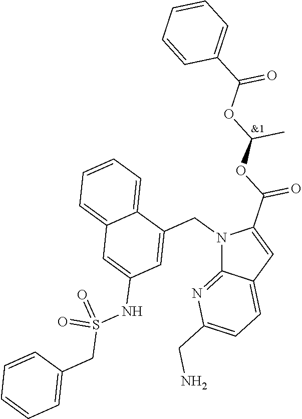 [SMILES: C[C@H](OC(=O)c1ccccc1)OC(=O)c1cc2ccc(CN)nc2n1Cc1cc(NS(=O)(=O)Cc2ccccc2)cc2ccccc12] | 1-[6-(aminomethyl)-1- {[3-(phenylmethanesul- fonamido)naphthalen-1- yl]methyl}-1H-pyrrolo- [2,3-b]pyridine-2-car- bonyloxy]ethyl benzoate | 648.7 | 100 | C |
| 125 | 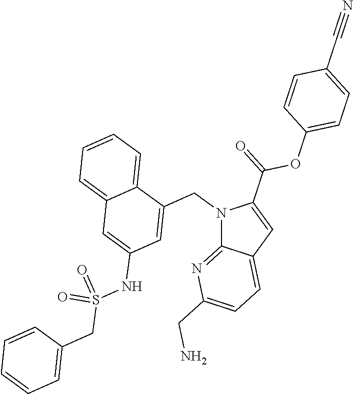 [SMILES: N#Cc1ccc(OC(=O)c2cc3ccc(CN)nc3n2Cc2cc(NS(=O)(=O)Cc3ccccc3)cc3ccccc23)cc1] | 4-cyanophenyl 6- (aminomethyl)-1-{[3- (phenylmethanesulfon- amido)naphthalen-1-yl]- methyl}-1H-pyrrolo- [2,3-b]pyridine-2-car- boxylate | 601.7 | 100 | C |
| 126 | 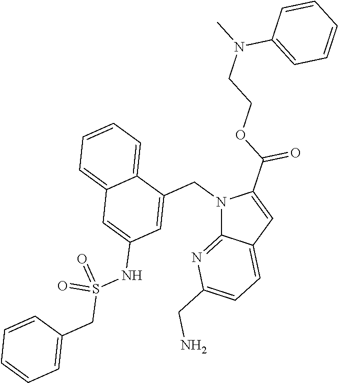 [SMILES: CN(CCOC(=O)c1cc2ccc(CN)nc2n1Cc1cc(NS(=O)(=O)Cc2ccccc2)cc2ccccc12)c1ccccc1] | 2-[methyl(phenyl)- amino]ethyl 6-(amino- methyl)-1-{[3-(phenyl- methanesulfonamido)- naphthalen-1-yl]methyl}- 1H-pyrrolo[2,3-b]pyr- idine-2-carboxylate | 634.2 | 98 | C |
| 127 | 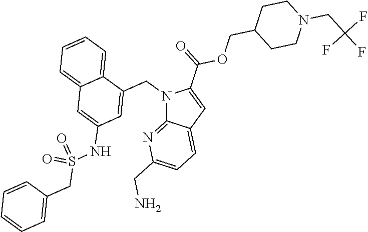 [SMILES: NCc1ccc2cc(C(=O)OCC3CCN(CC(F)(F)F)CC3)n(Cc3cc(NS(=O)(=O)Cc4ccccc4)cc4ccccc34)c2n1] | [1-(2,2,2-trifluoroethyl)- piperidin-4-yl]methyl 6- (aminomethyl)-1-{[3- (phenylmethanesulfon- amido)naphthalen-1- ylimethyl}-1H-pyrrolo- [2,3-b]pyridine-2-car- boxylate | 680.0 | 86 | |
| 128 | 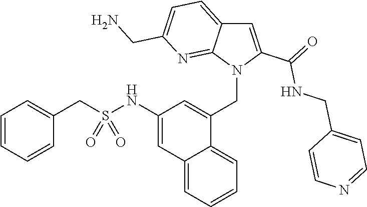 [SMILES: NCc1ccc2cc(C(=O)NCc3ccncc3)n(Cc3cc(NS(=O)(=O)Cc4ccccc4)cc4ccccc34)c2n1] | 6-(aminomethyl)-1-{[3- (phenylmethanesulfon- amido)naphthalen-1-yl]- methyl}-N-[(pyridin-4- yl)methyl]-1H-pyrrolo- [2,3-b]pyridine-2-car- boxamide | 591.0 | 100 | |
| 129 | 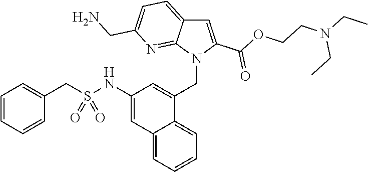 [SMILES: CCN(CC)CCOC(=O)c1cc2ccc(CN)nc2n1Cc1cc(NS(=O)(=O)Cc2ccccc2)cc2ccccc12] | 2-(diethylamino)ethyl 6-(aminomethyl)-1-{[3- (phenylmethanesulfon- amido)naphthalen-1-yl]- methyl}-1H-pyrrolo[2,3- b]pyridine-2-carboxylate | 600.0 | 93 | |
| 130 | 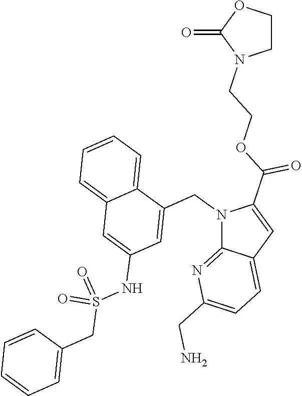 [SMILES: NCc1ccc2cc(C(=O)OCCN3CCOC3=O)n(Cc3cc(NS(=O)(=O)Cc4ccccc4)cc4ccccc34)c2n1] | 2-(2-oxo-1,3-oxazolidin- 3-yl)ethyl 6-(amino- methyl)-1-{[3-(phenyl- methanesulfonamido)- naphthalen-1-yl]meth- yl]}-1H-pyrrolo[2,3-b]- pyridine-2-carboxylate | 614.0 | 100 | B |
| 131 | 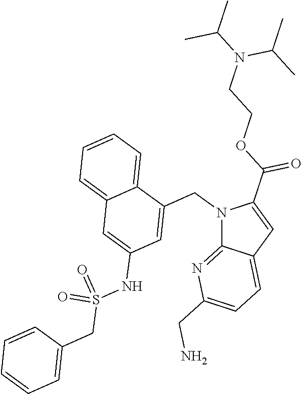 [SMILES: CC(C)N(CCOC(=O)c1cc2ccc(CN)nc2n1Cc1cc(NS(=O)(=O)Cc2ccccc2)cc2ccccc12)C(C)C] | 2-[bis(propan-2-yl)- amino]ethyl 6-(amino- methyl)-1-{[3-(phenyl- methanesulfonamido)- naphthalen-1-yl]meth- yl}-1H-pyrrolo[2,3-b]- pyridine-2-carboxylate | 628.0 | 95 | B |
| 132 | 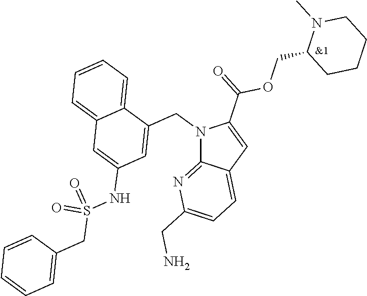 [SMILES: CN1CCCC[C@@H]1COC(=O)c1cc2ccc(CN)nc2n1Cc1cc(NS(=O)(=O)Cc2ccccc2)cc2ccccc12] | rac-[(2R)-1-methyl- piperidin-2-yl]methyl 6- (aminomethyl)-1-{[3- (phenylmethanesulfon- amido)naphthalen-1-yl]- methyl}-1H-pyrrolo[2,3- b]pyridine-2-carboxylate | 612.0 | 97 | A |
| 133 | 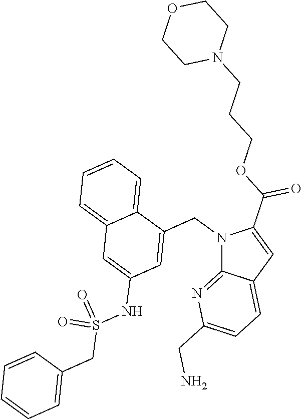 [SMILES: NCc1ccc2cc(C(=O)OCCCN3CCOCC3)n(Cc3cc(NS(=O)(=O)Cc4ccccc4)cc4ccccc34)c2n1] | 3-(morpholin-4-yl)prop- yl 6-(aminomethyl)-1- {[3-(phenylmethanesul- fonamido)naphthalen-1- ylmethyl}-1H-pyrrolo- [2,3-b]pyridine-2-car- boxylate | 628.0 | 99 | B |
| 134 | 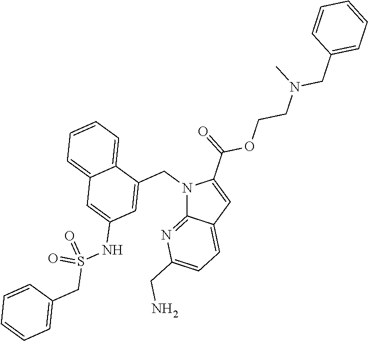 [SMILES: CN(CCOC(=O)c1cc2ccc(CN)nc2n1Cc1cc(NS(=O)(=O)Cc2ccccc2)cc2ccccc12)Cc1ccccc1] | 2-[benzyl(methyl)- amino]ethyl 6-(amino- methyl)-1-{[3-(phenyl- methanesulfonamido)- naphthalen-1-yl]methyl}- 1H-pyrrolo[2,3-b]pyr- idine-2-carboxylate | 648.0 | 85 | B |
| 135 | 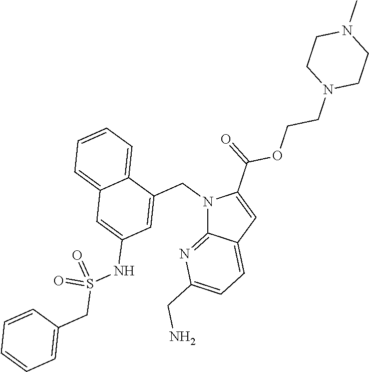 [SMILES: CN1CCN(CCOC(=O)c2cc3ccc(CN)nc3n2Cc2cc(NS(=O)(=O)Cc3ccccc3)cc3ccccc23)CC1] | 2-(4-methylpiperazin-1- ylethyl 6-(aminomethyl)- 1-{[3-(phenylmethane- sulfonamido)naphthalen- 1-yl]methyl}-1H-pyrrolo- [2,3-b]pyridine-2-car- boxylate | 627.0 | 99 | A |
| 136 | 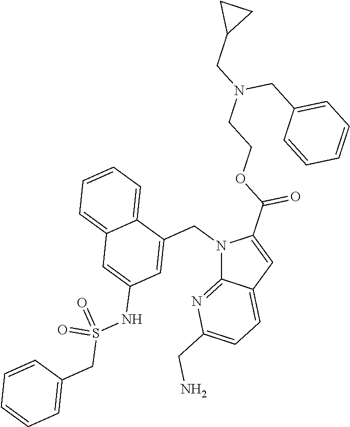 [SMILES: NCc1ccc2cc(C(=O)OCCN(Cc3ccccc3)CC3CC3)n(Cc3cc(NS(=O)(=O)Cc4ccccc4)cc4ccccc34)c2n1] | 2-[benzyl(cyclopropyl- methyl)amino]ethyl 6- (aminomethyl)-1-{[3- (phenylmethanesulfon- amido)naphthalen-1-yl]- methyl}-1H-pyrrolo[2,3- b]pyridine-2-carboxylate | 688.0 | 89 | C |
| 137 | 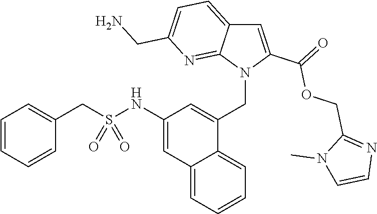 [SMILES: Cn1ccnc1COC(=O)c1cc2ccc(CN)nc2n1Cc1cc(NS(=O)(=O)Cc2ccccc2)cc2ccccc12] | (1-methyl-1H-imidazol- 2-yl)methyl 6-(amino- methyl)-1-{[3-(phenyl- methanesulfonamido)- naphthalen-1-yl]methyl}- 1H-pyrrolo[2,3-b]pyr- idine-2-carboxylate | 595.0 | 100 | A |
| 138 | 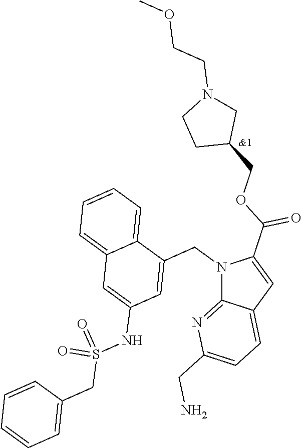 [SMILES: COCCN1CC[C@H](COC(=O)c2cc3ccc(CN)nc3n2Cc2cc(NS(=O)(=O)Cc3ccccc3)cc3ccccc23)C1] | rac-[(3R)-1-(2-methoxy- ethyl)pyrrolidin-3-yl]- methyl 6-(aminometh- yl)-1-{[3-(phenylmeth- anesulfonamido)naph- thalen-1-yl]methyl}-1H- pyrrolo[2,3-b]pyridine- 2-carboxylate | 642.0 | 99 | A |
| 139 | 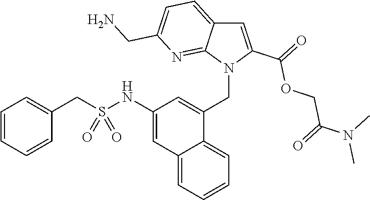 [SMILES: CN(C)C(=O)COC(=O)c1cc2ccc(CN)nc2n1Cc1cc(NS(=O)(=O)Cc2ccccc2)cc2ccccc12] | (dimethylcarbamoyl)- methyl 6-(aminomethyl)- 1-{[3-(phenylmethane- sulfonamido)naphthalen- 1-yl]methyl}-1H-pyrrolo- [2,3-b]pyridine-2-car- boxylate | 586.0 | 98 | B |
| 140 | 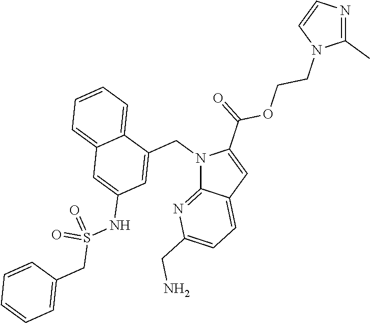 [SMILES: Cc1nccn1CCOC(=O)c1cc2ccc(CN)nc2n1Cc1cc(NS(=O)(=O)Cc2ccccc2)cc2ccccc12] | 2-(2-methyl-1H-imid- azol-1-yl)ethyl 6-(amino- methyl)-1-{[3-(phenyl- methanesulfonamido)- naphthalen-1-yl]methyl}- 1H-pyrrolo[2,3-b]pyr- idine-2-carboxylate | 609.0 | 100 | B |
| 141 | 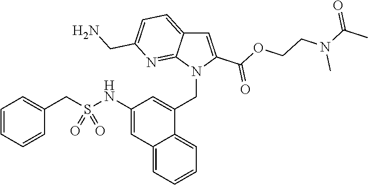 [SMILES: CC(=O)N(C)CCOC(=O)c1cc2ccc(CN)nc2n1Cc1cc(NS(=O)(=O)Cc2ccccc2)cc2ccccc12] | 2-(N-methylacetamido)- ethyl 6-(aminomethyl)- 1-{[3-(phenylmethane- sulfonamido)naphthalen- 1-yl]methyl}-1H-pyrrolo- [2,3-b]pyridine-2-car- boxylate | 600.0 | 95 | B |
| 142 | 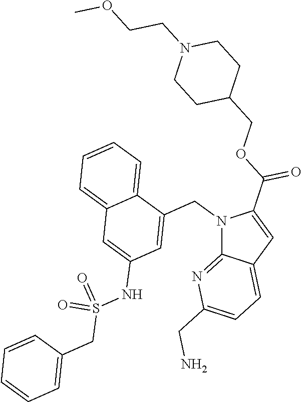 [SMILES: COCCN1CCC(COC(=O)c2cc3ccc(CN)nc3n2Cc2cc(NS(=O)(=O)Cc3ccccc3)cc3ccccc23)CC1] | [1-(2-methoxyethyl)- piperidin-4-yl]methyl 6- (aminomethyl)-1-{[3- (phenylmethanesulfon- amido)naphthalen-1-yl]- methyl}-1H-pyrrolo[2,3- b]pyridine-2-carboxylate | 656.0 | 100 | B |
| 143 | 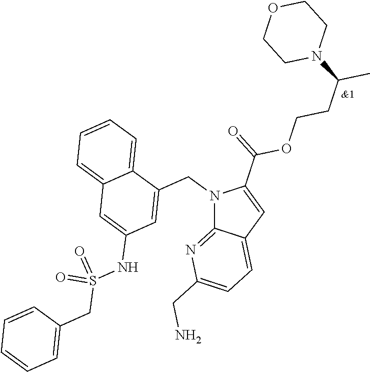 [SMILES: C[C@@H](CCOC(=O)c1cc2ccc(CN)nc2n1Cc1cc(NS(=O)(=O)Cc2ccccc2)cc2ccccc12)N1CCOCC1] | rac-(3R)-3-(morpholin-4- yl)butyl 6-(aminometh- yl)-1-{[3-(phenylmeth- anesulfonamido)naph- thalen-1-yl]methyl}-1H- pyrrolo-[2,3-b]pyridine- 2-carboxylate | 642.0 | 100 | A |
| 144 | 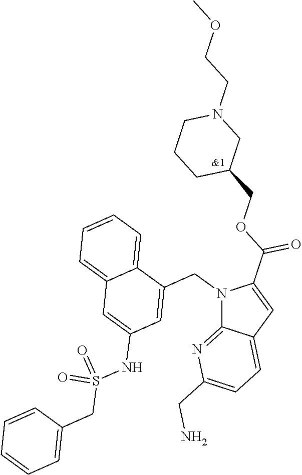 [SMILES: COCCN1CCC[C@H](COC(=O)c2cc3ccc(CN)nc3n2Cc2cc(NS(=O)(=O)Cc3ccccc3)cc3ccccc23)C1] | rac-[(3R)-1-(2-methoxy- ethyl)piperidin-3-yl]- methyl 6-(aminometh- yl)-1-(phenylmethane- sulfonamido)naphthalen- 1-yl]methyl}-1H-pyrrolo- [2,3-b]pyridine-2-car- boxylate | 656.0 | 96 | A |
| 145 | 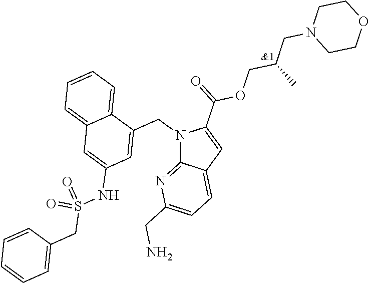 [SMILES: C[C@H](COC(=O)c1cc2ccc(CN)nc2n1Cc1cc(NS(=O)(=O)Cc2ccccc2)cc2ccccc12)CN1CCOCC1] | rac-(2R)-2-methyl-3- (morpholin-4-yl)propyl 6-(aminomethyl)-1-{[3- (phenylmethanesulfon- amido)naphthalen-1-yl]- methyl}-1H-pyrrolo[2,3- b]pyridine-2-carboxylate | 642.0 | 99 | A |
| 146 | 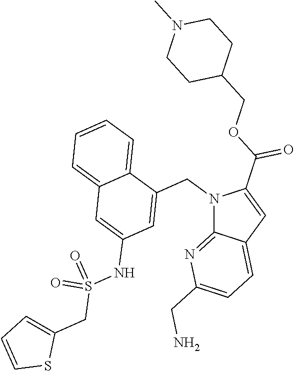 [SMILES: CN1CCC(COC(=O)c2cc3ccc(CN)nc3n2Cc2cc(NS(=O)(=O)Cc3cccs3)cc3ccccc23)CC1] | (1-methylpiperidin-4-yl)- methyl 6-(aminometh- yl)-1-({3-[(thiophen-2- yl)methanesulfonamido]- naphthalen-1-yl}methyl)- 1H-pyrrolo[2,3-b]pyr- idine-2-carboxylate | 618.4 | 91 | A |
| 147 | 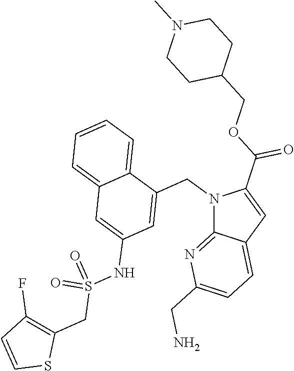 [SMILES: CN1CCC(COC(=O)c2cc3ccc(CN)nc3n2Cc2cc(NS(=O)(=O)Cc3sccc3F)cc3ccccc23)CC1] | (1-methylpiperidin-4-yl)- methyl 6-(aminomethyl)- 1-({3-[(3-fluorothiophen- 2-yl)methanesulfon- amido]naphthalen-1-yl}- methyl)-1H-pyrrolo[2,3- b]pyridine-2-carboxylate | 634.2 | 99 | A |
| 148 | 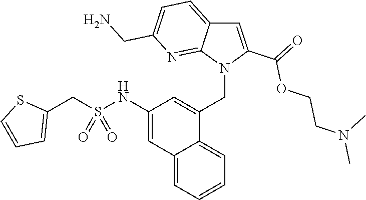 [SMILES: CN(C)CCOC(=O)c1cc2ccc(CN)nc2n1Cc1cc(NS(=O)(=O)Cc2cccs2)cc2ccccc12] | 2-(dimethylamino)ethyl 6-(aminomethyl)-1-({3- [(thiophen-2-yl)methane- sulfonamido]naphthalen- 1-yl}methyl)-1H-pyr- rolo[2,3-b]pyridine-2- carboxylate | 578.1 | 97 | A |
| 149 | 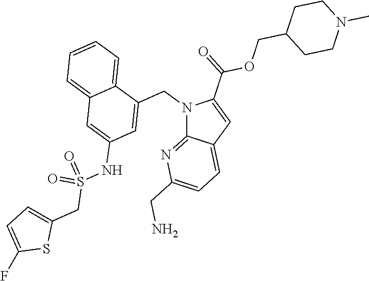 [SMILES: CN1CCC(COC(=O)c2cc3ccc(CN)nc3n2Cc2cc(NS(=O)(=O)Cc3ccc(F)s3)cc3ccccc23)CC1] | (1-methylpiperidin-4- yl)methyl 6-(amino- methyl)-1-({3-[(5-fluoro- thiophen-2-yl)methane- sulfonamido]naphthalen- 1-yl}methyl)-1H-pyr- rolo[2,3-b]pyridine-2- carboxylate | 634.1 | 97 | A |
| 150 | 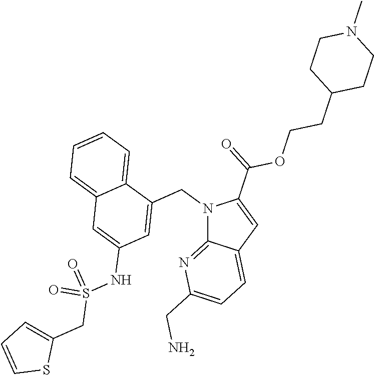 [SMILES: CN1CCC(CCOC(=O)c2cc3ccc(CN)nc3n2Cc2cc(NS(=O)(=O)Cc3cccs3)cc3ccccc23)CC1] | 2-(1-methylpiperidin-4- yl)ethyl 6-(aminometh- yl)-1-({3-[(thiophen-2- yl)methanesulfonamido]- naphthalen-1-yl}methyl)- 1H-pyrrolo[2,3-b]pyr- idine-2-carboxylate | 632.2 | 100 | A |
| 151 | 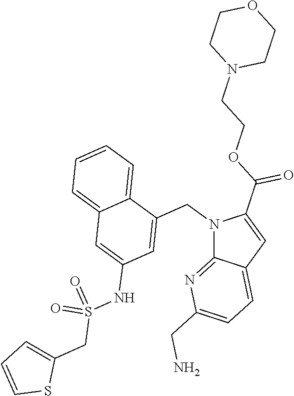 [SMILES: NCc1ccc2cc(C(=O)OCCN3CCOCC3)n(Cc3cc(NS(=O)(=O)Cc4cccs4)cc4ccccc34)c2n1] | 2-(morpholin-4-yl)ethyl 6-(aminomethyl)-1-({3- [(thiophen-2-yl)methane- sulfonamido]naphthalen- 1-yl}methyl)-1H-pyr- rolo[2,3-b]pyridine-2- carboxylate | 620.1 | 96 | A |
| 152 | 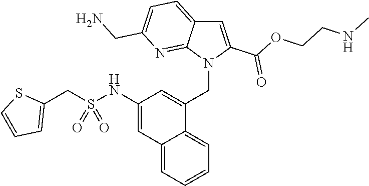 [SMILES: CNCCOC(=O)c1cc2ccc(CN)nc2n1Cc1cc(NS(=O)(=O)Cc2cccs2)cc2ccccc12] | 2-(methylamino)ethyl 6- (aminomethyl)-1-({3- [(thiophen-2-yl)methane- sulfonamido]naphthalen- 1-yl}methyl)-1H-pyr- rolo[2,3-b]pyridine-2- carboxylate | 564.2 | 95 | A |
| 153 | 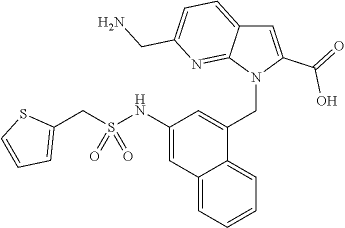 [SMILES: NCc1ccc2cc(C(=O)O)n(Cc3cc(NS(=O)(=O)Cc4cccs4)cc4ccccc34)c2n1] | 6-(aminomethyl)-1-({3- [(thiophen-2-yl)methane- sulfonamido]naphthalen- 1-yl}methyl)-1H-pyr- rolo[2,3-b]pyridine-2- carboxylic acid | 507.1 | 99 | A |
| 154 | 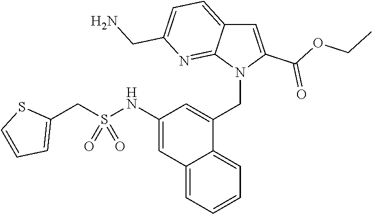 [SMILES: CCOC(=O)c1cc2ccc(CN)nc2n1Cc1cc(NS(=O)(=O)Cc2cccs2)cc2ccccc12] | ethyl 6-(aminomethyl)- 1-({3-[(thiophen-2-yl)- methanesulfonamido]- naphthalen-1-yl}methyl)- 1H-pyrrolo[2,3-b]pyr- idine-2-carboxylate | 535.3 | 100 | B |
| 155 | 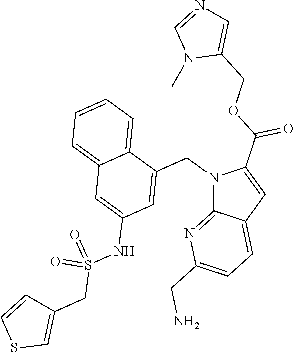 [SMILES: Cn1cncc1COC(=O)c1cc2ccc(CN)nc2n1Cc1cc(NS(=O)(=O)Cc2ccsc2)cc2ccccc12] | (1-methyl-1H-imidazol- 5-yl)methyl 6-(amino- methyl)-1-({3-[(thiophen- 3-yl)methanesulfon- amido]naphthalen-1-yl}- methyl)-1H-pyrrolo[2,3- b]pyridine-2-carboxylate | 600.9 | 100 | A |
| 156 | 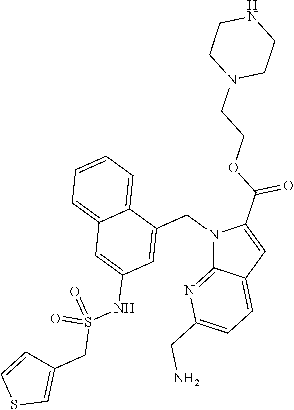 [SMILES: NCc1ccc2cc(C(=O)OCCN3CCNCC3)n(Cc3cc(NS(=O)(=O)Cc4ccsc4)cc4ccccc34)c2n1] | 2-(piperazin-1-yl)ethyl 6-(aminomethyl)-1-({3- [(thiophen-3-yl)methane- sulfonamidoJnaphthalen- 1-yl}methyl)-1H-pyr- rolo[2,3-b]pyridine-2- carboxylate | 619.1 | 100 | A |
| 157 | 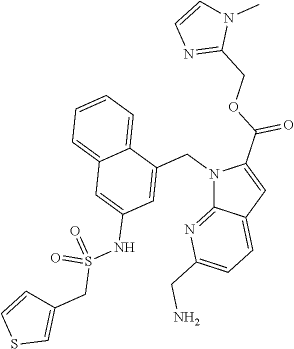 [SMILES: Cn1ccnc1COC(=O)c1cc2ccc(CN)nc2n1Cc1cc(NS(=O)(=O)Cc2ccsc2)cc2ccccc12] | (1-methyl-1H-imidazol- 2-yl)methyl 6-amino- methyl)-1-({3-[(thio- phen-3-yl)methanesul- fonamido]naphthalen-1- yl}methyl)-1H-pyrrolo- [2,3-b]pyridine-2-car- boxylate | 599.1 [M − H] | 99 | A |
| 158 | 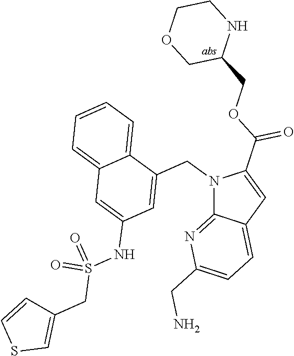 [SMILES: NCc1ccc2cc(C(=O)OC[C@H]3COCCN3)n(Cc3cc(NS(=O)(=O)Cc4ccsc4)cc4ccccc34)c2n1] | [(3R)-morpholin-3-yl]- methyl 6-(aminometh- yl)-1-({3-[(thiophen-3- yl)methanesulfonamido]- naphthalen-1-yl}meth- yl)-1H-pyrrolo[2,3-b]- pyridine-2-carboxylate | 606.3 | 98 | A |
| 159 | 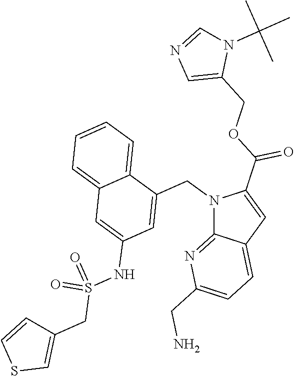 [SMILES: CC(C)(C)n1cncc1COC(=O)c1cc2ccc(CN)nc2n1Cc1cc(NS(=O)(=O)Cc2ccsc2)cc2ccccc12] | (1-tert-butyl-1H-imid- azol-5-yl)methyl 6- (aminomethyl)-1-({3- [(thiophen-3-yl)methane- sulfonamido]naphthalen- 1-yl}methyl)-1H-pyr- rolo[2,3-b]pyridine-2- carboxylate | 643.3 | 99 | A |
| 160 | 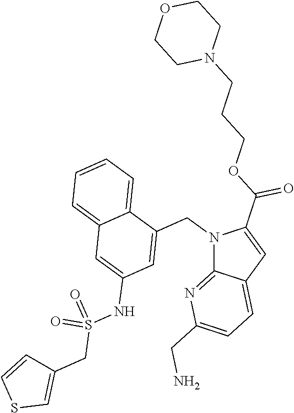 [SMILES: NCc1ccc2cc(C(=O)OCCCN3CCOCC3)n(Cc3cc(NS(=O)(=O)Cc4ccsc4)cc4ccccc34)c2n1] | 3-(morpholin-4-yl)prop- yl 6-(aminomethyl)-1- ({3-[(thiophen-3-yl)meth- anesulfonamido]naph- thalen-1-yl}methyl)-1H- pyrrolo[2,3-b]pyridine- 2-carboxylate | 634.0 | 100 | A |
| 161 | 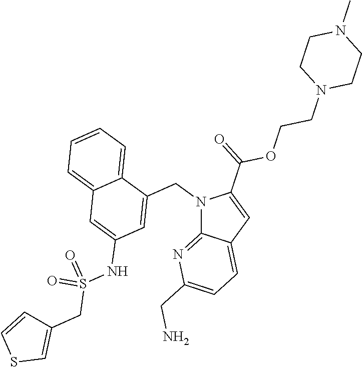 [SMILES: CN1CCN(CCOC(=O)c2cc3ccc(CN)nc3n2Cc2cc(NS(=O)(=O)Cc3ccsc3)cc3ccccc23)CC1] | 2-(4-methylpiperazin-1- yl)ethyl 6-(aminometh- yl)-1-({3-[(thiophen-3- yl)methanesulfonamido]- naphthalen-1-yl}meth- yl)-1H-pyrrolo[2,3-b]- pyridine-2-carboxylate | 633.4 | 99 | A |
| 162 | 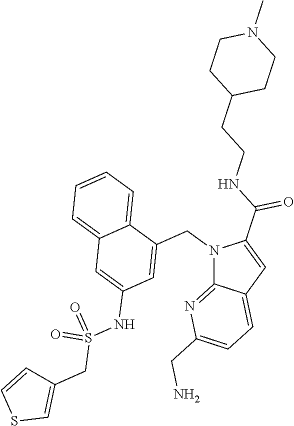 [SMILES: CN1CCC(CCNC(=O)c2cc3ccc(CN)nc3n2Cc2cc(NS(=O)(=O)Cc3ccsc3)cc3ccccc23)CC1] | 6-(aminomethyl)-N-[2- (1-methylpiperidin-4-yl)- ethyl]-1-({3-[(thiophen- 3-yl)methanesulfon- amido]naphthalen-1-yl}- methyl)-1H-pyrrolo[2,3- b]pyridine-2-carboxamide | 631.0 | 96 | A |
| 163 | 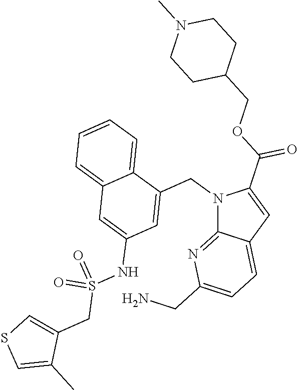 [SMILES: Cc1cscc1CS(=O)(=O)Nc1cc(Cn2c(C(=O)OCC3CCN(C)CC3)cc3ccc(CN)nc32)c2ccccc2c1] | (1-methylpiperidin-4-yl)- methyl 6-(aminomethyl)- 1-({3-[(4-methylthio- phen-3-yl)methanesul- fonamido]naphthalen-1- yl}methyl)-1H-pyrrolo- [2,3-b]pyridine-2-car- boxylate | 632.4 | 98 | A |
| 164 | 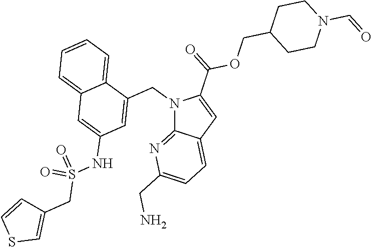 [SMILES: NCc1ccc2cc(C(=O)OCC3CCN(C=O)CC3)n(Cc3cc(NS(=O)(=O)Cc4ccsc4)cc4ccccc34)c2n1] | (1-formylpiperidin-4- yl)methyl 6-(aminometh- yl)-1-({3-[(thiophen-3- yl)methanesulfonamido]- naphthalen-1-yl}methyl)- 1H-pyrrolo[2,3-b]pyr- idine-2-carboxylate | 632.3 | 100 | A |
| 165 | 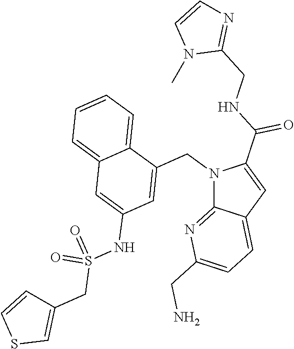 [SMILES: Cn1ccnc1CNC(=O)c1cc2ccc(CN)nc2n1Cc1cc(NS(=O)(=O)Cc2ccsc2)cc2ccccc12] | 6-(aminomethyl)-N-[(1- methyl-1H-imidazol-2- yl)methyl]-1-({3-[(thio- phen-3-yl)methanesul- fonamidoJnaphthalen-1- yl}methyl)-1H-pyrrolo- [2,3-b]pyridine-2-car- boxamide | 600.3 | 99 | A |
| 166 | 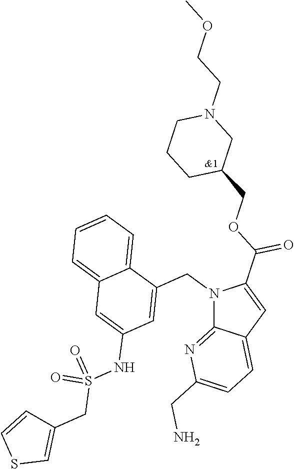 [SMILES: COCCN1CCC[C@H](COC(=O)c2cc3ccc(CN)nc3n2Cc2cc(NS(=O)(=O)Cc3ccsc3)cc3ccccc23)C1] | [1-(2-methoxyethyl)- piperidin-3-yl]methyl 6- (aminomethyl)-1-({3- [(thiophen-3-yl)methane- sulfonamido]naphthalen- 1-yl}methyl)-1H-pyr- rolo[2,3-b]pyridine-2- carboxylate | 660.2 [M − H] | 97 | A |
| 167 | 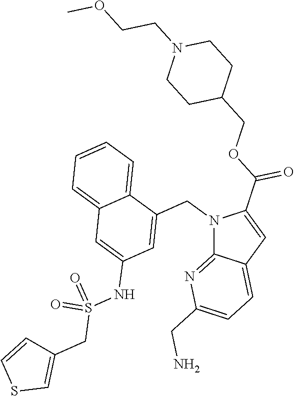 [SMILES: COCCN1CCC(COC(=O)c2cc3ccc(CN)nc3n2Cc2cc(NS(=O)(=O)Cc3ccsc3)cc3ccccc23)CC1] | [1-(2-methoxyethyl)- piperidin-4-yl]methyl 6- (aminomethyl)-1-({3- [(thiophen-3-yl)meth- anesulfonamido]naph- thalen-1-yl}methyl)-1H- pyrrolo[2,3-b]pyridine- 2-carboxylate | 662.0 | 98 | A |
| 168 | 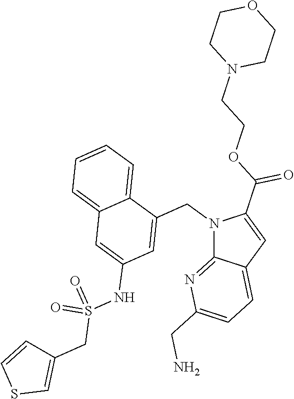 [SMILES: NCc1ccc2cc(C(=O)OCCN3CCOCC3)n(Cc3cc(NS(=O)(=O)Cc4ccsc4)cc4ccccc34)c2n1] | 2-(morpholin-4-yl)ethyl 6-(aminomethyl)-1-({3- [(thiophen-3-yl)methane- sulfonamido]naphthalen- 1-yl}methyl)-1H-pyr- rolo[2,3-b]pyridine-2- carboxylate | 620.1 | 96 | A |
| 169 | 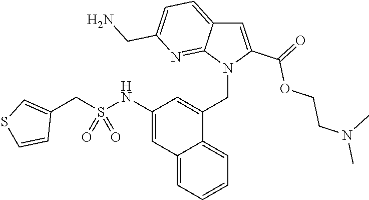 [SMILES: CN(C)CCOC(=O)c1cc2ccc(CN)nc2n1Cc1cc(NS(=O)(=O)Cc2ccsc2)cc2ccccc12] | 2-(dimethylamino)ethyl 6-(aminomethyl)-1-({3- [(thiophen-3-yl)methane- sulfonamido]naphthalen- 1-yl}methyl)-1H-pyr- rolo[2,3-b]pyridine-2- carboxylate | 578.3 | 99 | A |
| 170 | 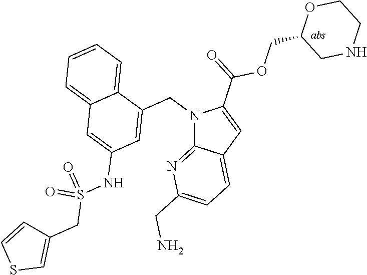 [SMILES: NCc1ccc2cc(C(=O)OC[C@H]3CNCCO3)n(Cc3cc(NS(=O)(=O)Cc4ccsc4)cc4ccccc34)c2n1] | [(2R)-morpholin-2-yl]- methyl 6-(aminomethyl)- 1-({3-[(thiophen-3-yl)- methanesulfonamido]- naphthalen-1-yl}meth- yl)-1H-pyrrolo[2,3-b]- pyridine-2-carboxylate | 606.1 | 99 | A |
| 171 | 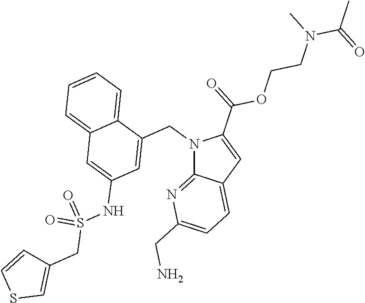 [SMILES: CC(=O)N(C)CCOC(=O)c1cc2ccc(CN)nc2n1Cc1cc(NS(=O)(=O)Cc2ccsc2)cc2ccccc12] | 2-(N-methylacetamido)- ethyl 6-(aminomethyl)- 1-({3-[(thiophen-3-yl)- methanesulfonamido]- naphthalen-1-yl}meth- yl)-1H-pyrrolo[2,3-b]- pyridine-2-carboxylate | 605.9 | 99 | A |
| 172 | 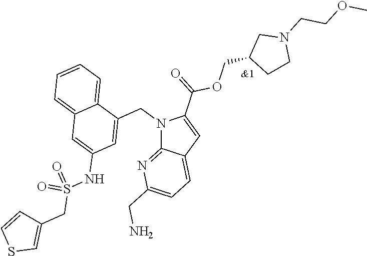 [SMILES: COCCN1CC[C@H](COC(=O)c2cc3ccc(CN)nc3n2Cc2cc(NS(=O)(=O)Cc3ccsc3)cc3ccccc23)C1] | [1-(2-methoxyethyl)- pyrrolidin-3-yl]methyl 6-(aminomethyl)-1-({3- [(thiophen-3-yl)methane- sulfonamido]naphthalen- 1-yl}methyl)-1H-pyr- rolo[2,3-b]pyridine-2- carboxylate | 646.0 [M − H] | 96 | A |
| 173 | 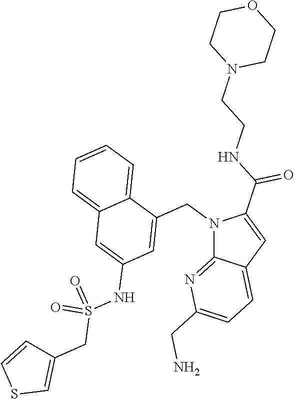 [SMILES: NCc1ccc2cc(C(=O)NCCN3CCOCC3)n(Cc3cc(NS(=O)(=O)Cc4ccsc4)cc4ccccc34)c2n1] | 6-(aminomethyl)-N-[2- (morpholin-4-yl)ethyl]- 1-({3-[(thiophen-3-yl)- methanesulfonamido]- naphthalen-1-yl}meth- yl)-1H-pyrrolo[2,3-b]- pyridine-2-carboxamide | 619.0 | 99 | A |
| 174 | 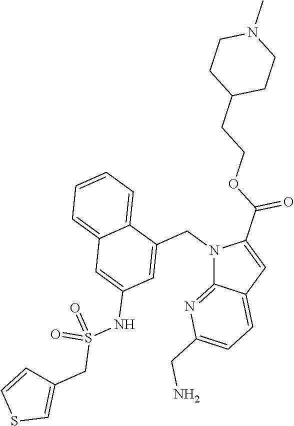 [SMILES: CN1CCC(CCOC(=O)c2cc3ccc(CN)nc3n2Cc2cc(NS(=O)(=O)Cc3ccsc3)cc3ccccc23)CC1] | 2-(1-methylpiperidin-4- ylethyl 6-(aminomethyl)- 1-({3-[(thiophen-3-yl)- methanesulfonamido]- naphthalen-1-yl}meth- yl)-1H-pyrrolo[2,3-b]- pyridine-2-carboxylate | 632.1 | 95 | A |
| 175 | 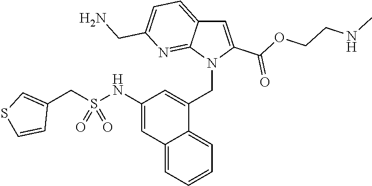 [SMILES: CNCCOC(=O)c1cc2ccc(CN)nc2n1Cc1cc(NS(=O)(=O)Cc2ccsc2)cc2ccccc12] | 2-(methylamino)ethyl 6- (aminomethyl)-1-({3- [(thiophen-3-yl)methane- sulfonamido]naphthalen- 1-yl}methyl)-1H-pyr- rolo[2,3-b]pyridine-2- carboxylate | 564.3 | 99 | A |
| 176 | 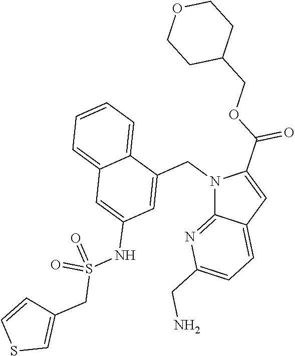 [SMILES: NCc1ccc2cc(C(=O)OCC3CCOCC3)n(Cc3cc(NS(=O)(=O)Cc4ccsc4)cc4ccccc34)c2n1] | (oxan-4-yl)methyl 6- (aminomethyl)-1-({3- [(thiophen-3-yl)methane- sulfonamido]naphthalen- 1-yl}methyl)-1H-pyr- rolo[2,3-b]pyridine-2- carboxylate | 605.0 | 100 | A |
| 177 | 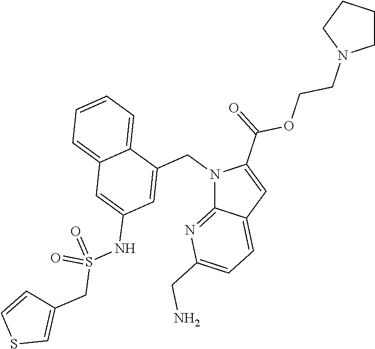 [SMILES: NCc1ccc2cc(C(=O)OCCN3CCCC3)n(Cc3cc(NS(=O)(=O)Cc4ccsc4)cc4ccccc34)c2n1] | 2-(pyrrolidin-1-yl)ethyl 6-(aminomethyl)-1-({3- [(thiophen-3-yl)methane- sulfonamido]naphthalen- 1-yl}methyl)-1H-pyr- rolo[2,3-b]pyridine-2- carboxylate | 604.3 | 98 | A |
| 178 | 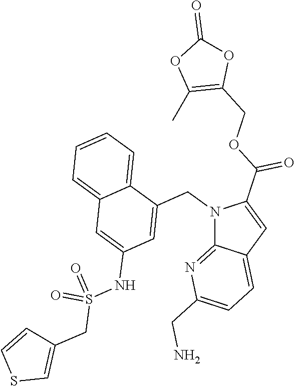 [SMILES: Cc1oc(=O)oc1COC(=O)c1cc2ccc(CN)nc2n1Cc1cc(NS(=O)(=O)Cc2ccsc2)cc2ccccc12] | (5-methyl-2-oxo-2H- 1,3-dioxol-4-yl)methyl 6-(aminomethyl)-1-({3- [(thiophen-3-yl)methane- sulfonamido]naphthalen- 1-yl}methyl)-1H-pyr- rolo[2,3-b]pyridine-2- carboxylate | 619.0 | 98 | B |
| 179 | 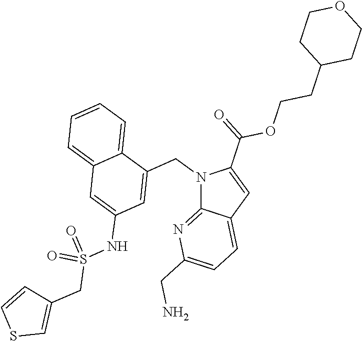 [SMILES: NCc1ccc2cc(C(=O)OCCC3CCOCC3)n(Cc3cc(NS(=O)(=O)Cc4ccsc4)cc4ccccc34)c2n1] | 2-(oxan-4-yl)ethyl 6- (aminomethyl)-1-({3- [(thiophen-3-yl)methane- sulfonamido]naphthalen- 1-yl}methyl)-1H-pyr- rolo[2,3-b]pyridine-2- carboxylate | 618.9 | 98 | B |
| 180 | 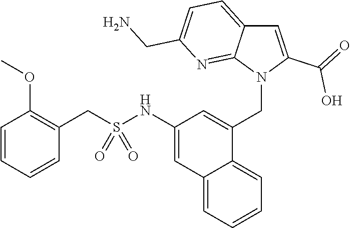 [SMILES: COc1ccccc1CS(=O)(=O)Nc1cc(Cn2c(C(=O)O)cc3ccc(CN)nc32)c2ccccc2c1] | 6-(aminomethyl)-1-({3- [(2-methoxyphenyl)- methanesulfonamido]- naphthalen-1-yl}meth- yl)-1H-pyrrolo[2,3-b]- pyridine-2-carboxylic acid | 531.1 | 98 | B |
| 181 | 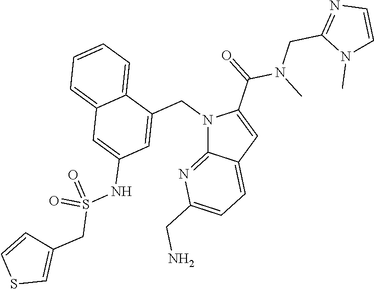 [SMILES: CN(Cc1nccn1C)C(=O)c1cc2ccc(CN)nc2n1Cc1cc(NS(=O)(=O)Cc2ccsc2)cc2ccccc12] | 6-(aminomethyl)-N- methyl-N-[(1-methyl- 1H-imidazol-2-yl)meth- yl]-1-({3-{(thiophen-3- yl)methanesulfonamido]- naphthalen-1-yl}methyl)- 1H-pyrrolo[2,3-b]pyr- idine-2-carboxamide | 614.4 | 97 | B |
| 182 | 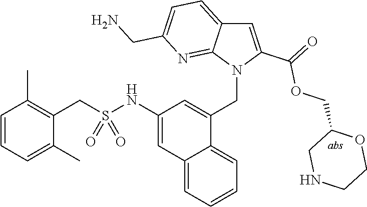 [SMILES: Cc1cccc(C)c1CS(=O)(=O)Nc1cc(Cn2c(C(=O)OC[C@H]3CNCCO3)cc3ccc(CN)nc32)c2ccccc2c1] | [(2R)-morpholin-2-yl]- methyl 6-(aminometh- yl)-1-({3-[(2,6-dimeth- ylphenyl)methanesulfon- amido]naphthalen-1-yl}- methyl)-1H-pyrrolo[2,3- b]pyridine-2-carboxylate | 628.4 | 99 | A |
| 183 | 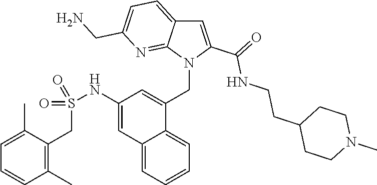 [SMILES: Cc1cccc(C)c1CS(=O)(=O)Nc1cc(Cn2c(C(=O)NCCC3CCN(C)CC3)cc3ccc(CN)nc32)c2ccccc2c1] | 6-(aminomethyl)-1-({3- [(2,6-dimethylphenyl)- methanesulfonamido]- naphthalen-1-yl}meth- yl)-N-[2-(1-methylpiper- idin-4-yl)ethyl]-1H-pyr- rolo[2,3-b]pyridine-2- carboxamide | 651.1 [M − H] | 99 | A |
| 184 | 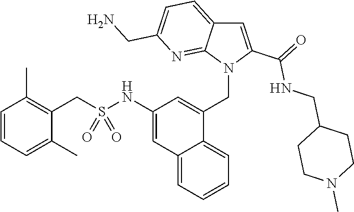 [SMILES: Cc1cccc(C)c1CS(=O)(=O)Nc1cc(Cn2c(C(=O)NCC3CCN(C)CC3)cc3ccc(CN)nc32)c2ccccc2c1] | 6-(aminomethyl)-1-({3- [(2,6-dimethylphenyl)- methanesulfonamido]- naphthalen-1-yl}meth- yl)-N-[(1-methylpiper- idin-4-yl)methyl]-1H- pyrrolo[2,3-b]pyridine- 2-carboxamide | 639.4 | 100 | A |
| 185 | 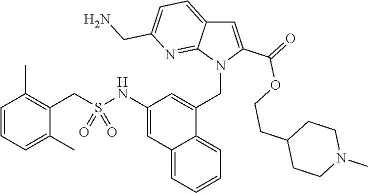 [SMILES: Cc1cccc(C)c1CS(=O)(=O)Nc1cc(Cn2c(C(=O)OCCC3CCN(C)CC3)cc3ccc(CN)nc32)c2ccccc2c1] | 2-(1-methylpiperidin-4- yl)ethyl 6-(aminometh- yl)-1-({3-[(2,6-dimethyl- phenyl)methanesulfon- amido]naphthalen-1-yl}- methyl)-1H-pyrrolo[2,3- b]pyridine-2-carboxylate | 654.4 | 97 | A |
| 186 | 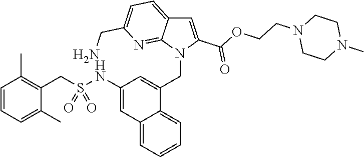 [SMILES: Cc1cccc(C)c1CS(=O)(=O)Nc1cc(Cn2c(C(=O)OCCN3CCN(C)CC3)cc3ccc(CN)nc32)c2ccccc2c1] | 2-(4-methylpiperazin-1- ylethyl 6-(aminometh- yl)-1-({3-[(2,6-dimethyl- phenyl)methanesulfon- amido]naphthalen-1-yl}- methyl)-1H-pyrrolo[2,3- b]pyridine-2-carboxylate | 655.4 | 99 | A |
| 187 | 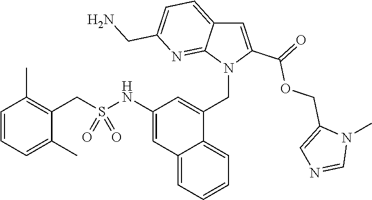 [SMILES: Cc1cccc(C)c1CS(=O)(=O)Nc1cc(Cn2c(C(=O)OCc3cncn3C)cc3ccc(CN)nc32)c2ccccc2c1] | (1-methyl-1H-imidazol- 5-yl)methyl 6-(amino- methyl)-1-({3-[(2,6- dimethylphenyl)methane- sulfonamido]naphthalen- 1-yl}methyl)-1H-pyr- rolo[2,3-b]pyridine-2- carboxylate | 623.4 | 99 | A |
| 188 | 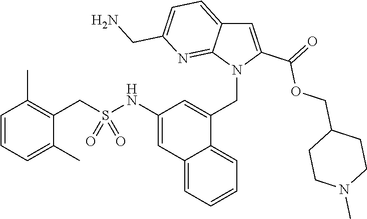 [SMILES: Cc1cccc(C)c1CS(=O)(=O)Nc1cc(Cn2c(C(=O)OCC3CCN(C)CC3)cc3ccc(CN)nc32)c2ccccc2c1] | (1-methylpiperidin-4- yl)methyl 6-(aminometh- yl)-1-({3-[(2,6-dimethyl- phenyl)methanesulfon- amido]naphthalen-1-yl}- methyl)-1H-pyrrolo[2,3- b]pyridine-2-carboxylate | 639.8 | 100 | A |
| 189 | 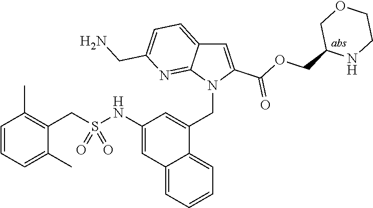 [SMILES: Cc1cccc(C)c1CS(=O)(=O)Nc1cc(Cn2c(C(=O)OC[C@H]3COCCN3)cc3ccc(CN)nc32)c2ccccc2c1] | [(3R)-morpholin-3-yl]- methyl 6-(aminomethyl)- 1-({3-[(2,6-dimethyl- phenyl)methanesulfon- amido]naphthalen-1-yl}- methyl)-1H-pyrrolo[2,3- b]pyridine-2-carboxylate | 628.4 | 99 | A |
| 190 | 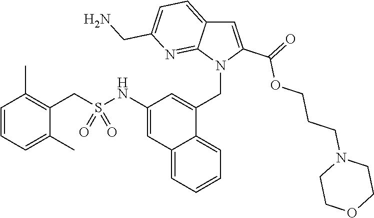 [SMILES: Cc1cccc(C)c1CS(=O)(=O)Nc1cc(Cn2c(C(=O)OCCCN3CCOCC3)cc3ccc(CN)nc32)c2ccccc2c1] | 3-(morpholin-4-yl)prop- yl 6-(aminomethyl)-1- ({3-[(2,6-dimethylphen- yl)methanesulfonamido]- naphthalen-1-yl}meth- yl)-1H-pyrrolo[2,3-b]- pyridine-2-carboxylate | 656.4 | 99 | A |
| 191 | 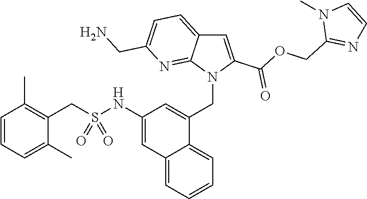 [SMILES: Cc1cccc(C)c1CS(=O)(=O)Nc1cc(Cn2c(C(=O)OCc3nccn3C)cc3ccc(CN)nc32)c2ccccc2c1] | (1-methyl-1H-imidazol- 2-yl)methyl 6-(amino- methyl)-1-({3-[(2,6- dimethylphenyl)meth- anesulfonamido]naph- thalen-1-yl}methyl)-1H- pyrrolo[2,3-b]pyridine- 2-carboxylate | 623.4 | 100 | A |
| 192 | 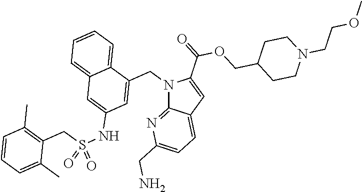 [SMILES: COCCN1CCC(COC(=O)c2cc3ccc(CN)nc3n2Cc2cc(NS(=O)(=O)Cc3c(C)cccc3C)cc3ccccc23)CC1] | [1-(2-methoxyethyl)- piperidin-4-yl]methyl 6-(aminomethyl)-1-({3- [(2,6-dimethylphenyl)- methanesulfonamido]- naphthalen-1-yl}meth- yl)-1H-pyrrolo[2,3-b]- pyridine-2-carboxylate | 684.4 | 100 | A |
| 193 | 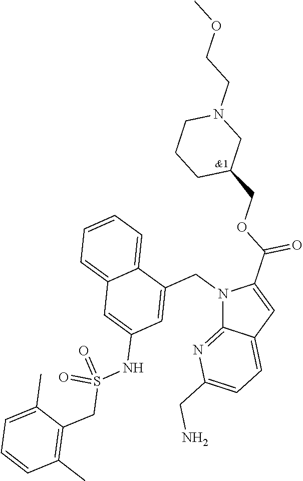 [SMILES: COCCN1CCC[C@H](COC(=O)c2cc3ccc(CN)nc3n2Cc2cc(NS(=O)(=O)Cc3c(C)cccc3C)cc3ccccc23)C1] | [1-(2-methoxyethyl)- piperidin-3-yl]methyl 6- (aminomethyl)-1-({3- [(2,6-dimethylphenyl)- methanesulfonamido]- naphthalen-1-yl}meth- yl)-1H-pyrrolo[2,3-b]- pyridine-2-carboxylate | 684.4 | 99 | A |
| 194 | 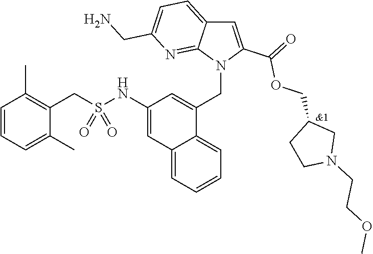 [SMILES: COCCN1CC[C@H](COC(=O)c2cc3ccc(CN)nc3n2Cc2cc(NS(=O)(=O)Cc3c(C)cccc3C)cc3ccccc23)C1] | [1-(2-methoxyethyl)pyr- rolidin-3-yl]methyl 6- (aminomethyl)-1-({3- [(2,6-dimethylphenyl)- methanesulfonamido]- naphthalen-1-yl}meth- yl)-1H-pyrrolo[2,3-b]- pyridine-2-carboxylate | 670.5 | 95 | A |
| 195 | 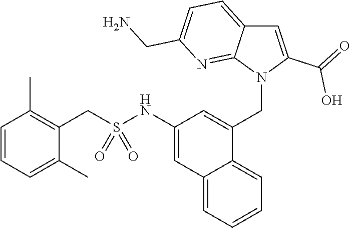 [SMILES: Cc1cccc(C)c1CS(=O)(=O)Nc1cc(Cn2c(C(=O)O)cc3ccc(CN)nc32)c2ccccc2c1] | 6-(aminomethyl)-1-({3- [(2,6-dimethylphenyl)- methanesulfonamido]- naphthalen-1-yl}methyl)- 1H-pyrrolo[2,3-b]pyr- idine-2-carboxylic acid | 529.1 | 100 | A |
| 196 | 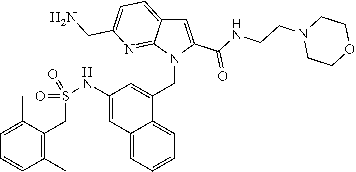 [SMILES: Cc1cccc(C)c1CS(=O)(=O)Nc1cc(Cn2c(C(=O)NCCN3CCOCC3)cc3ccc(CN)nc32)c2ccccc2c1] | 6-(aminomethyl)-1-({3- [(2,6-dimethylphenyl)- methanesulfonamido]- naphthalen-1-yl}meth- yl)-N-[2-(morpholin-4- yl)ethyl]-1H-pyrrolo- [2,3-b]pyridine-2-car- boxamide | 641.1 | 99 | A |
| 197 | 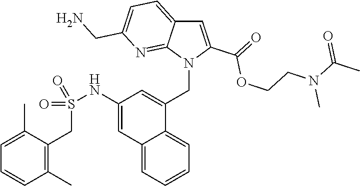 [SMILES: CC(=O)N(C)CCOC(=O)c1cc2ccc(CN)nc2n1Cc1cc(NS(=O)(=O)Cc2c(C)cccc2C)cc2ccccc12] | 2-(N-methylacetamido)- ethyl 6-(aminomethyl)-1- ({3-[(2,6-dimethylphen- yl)methanesulfonamido]- naphthalen-1-yl}methyl)- 1H-pyrrolo[2,3-b]pyr- idine-2-carboxylate | 628.5 | 98 | A |
| 198 | 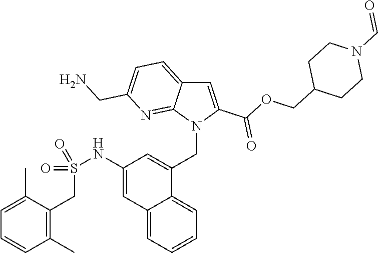 [SMILES: Cc1cccc(C)c1CS(=O)(=O)Nc1cc(Cn2c(C(=O)OCC3CCN(C=O)CC3)cc3ccc(CN)nc32)c2ccccc2c1] | (1-formylpiperidin-4-yl)- methyl 6-(aminomethyl)- 1-({3-[(2,6-dimethyl- phenyl)methanesulfon- amido]naphthalen-1-yl}- methyl)-1H-pyrrolo[2,3- b]pyridine-2-carboxylate | 654.5 | 97 | A |
| 199 | 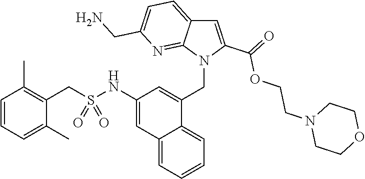 [SMILES: Cc1cccc(C)c1CS(=O)(=O)Nc1cc(Cn2c(C(=O)OCCN3CCOCC3)cc3ccc(CN)nc32)c2ccccc2c1] | 2-(morpholin-4-yl)ethyl 6-(aminomethyl)-1-({3- [(2,6-dimethylphenyl)- methanesulfonamido]- naphthalen-1-yl}meth- yl)-1H-pyrrolo[2,3-b]- pyridine-2-carboxylate | 642.1 | 100 | B |
| 200 | 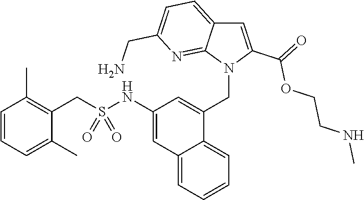 [SMILES: CNCCOC(=O)c1cc2ccc(CN)nc2n1Cc1cc(NS(=O)(=O)Cc2c(C)cccc2C)cc2ccccc12] | 2-(methylamino)ethyl 6-(aminomethyl)-1-({3- [(2,6-dimethylphenyl)- methanesulfonamido]- naphthalen-1-yl}meth- yl)-1H-pyrrolo[2,3-b]- pyridine-2-carboxylate | 586.4 | 99 | B |
| 201 | 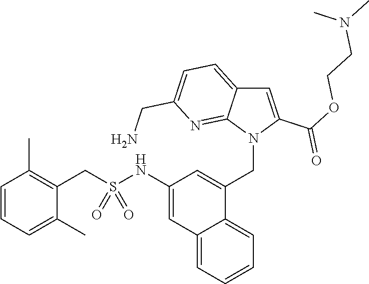 [SMILES: Cc1cccc(C)c1CS(=O)(=O)Nc1cc(Cn2c(C(=O)OCCN(C)C)cc3ccc(CN)nc32)c2ccccc2c1] | 2-(dimethylamino)ethyl 6-(aminomethyl)-1-({3- [(2,6-dimethylphenyl)- methanesulfonamido]- naphthalen-1-yl}methyl)- 1H-pyrrolo[2,3-b]pyr- idine-2-carboxylate | 600.4 | 98 | B |
| 202 | 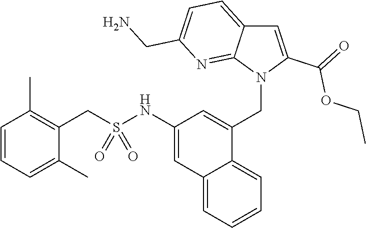 [SMILES: CCOC(=O)c1cc2ccc(CN)nc2n1Cc1cc(NS(=O)(=O)Cc2c(C)cccc2C)cc2ccccc12] | ethyl 6-(aminomethyl)- 1-({3-[(2,6-dimethyl- phenyl)methanesulfon- amido]naphthalen-1-yl}- methyl)-1H-pyrrolo[2,3- b]pyridine-2-carboxylate | 557.4 | 96 | C |
| 203 | 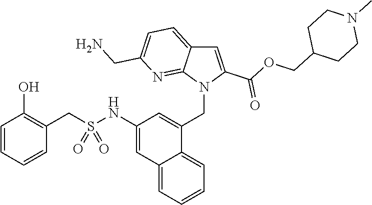 [SMILES: CN1CCC(COC(=O)c2cc3ccc(CN)nc3n2Cc2cc(NS(=O)(=O)Cc3ccccc3O)cc3ccccc23)CC1] | (1-methylpiperidin-4-yl)- methyl 6-(aminomethyl)- 1-({3-1(2-hydroxyphen- yl)methanesulfonamido]- naphthalen-1-yl}methyl)- 1H-pyrrolo[2,3-b]pyr- idine-2-carboxylate | 626.1 [M − H] | 93 | A |
| 204 | 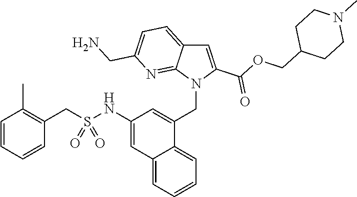 [SMILES: Cc1ccccc1CS(=O)(=O)Nc1cc(Cn2c(C(=O)OCC3CCN(C)CC3)cc3ccc(CN)nc32)c2ccccc2c1] | (1-methylpiperidin-4-yl)- methyl 6-(aminomethyl)- 1-({3-1(2-methylphen- yl)methanesulfonamido]- naphthalen-1-yl}meth- yl)-1H-pyrrolo[2,3-b]- pyridine-2-carboxylate | 626.4 | 99 | A |
| 205 | 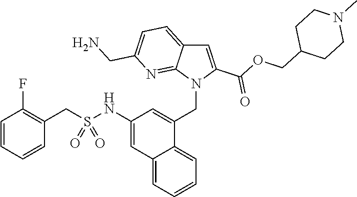 [SMILES: CN1CCC(COC(=O)c2cc3ccc(CN)nc3n2Cc2cc(NS(=O)(=O)Cc3ccccc3F)cc3ccccc23)CC1] | (1-methylpiperidin-4- yl)methyl 6-(aminometh- yl)-1-({3-[(2-fluorophen- yl)methanesulfonamido]- naphthalen-1-yl}methyl)- 1H-pyrrolo[2,3-b]pyr- idine-2-carboxylate | 630.4 | 97 | A |
| 206 | 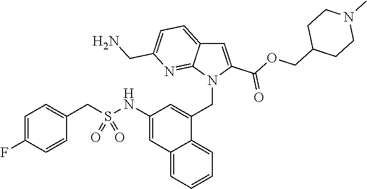 [SMILES: CN1CCC(COC(=O)c2cc3ccc(CN)nc3n2Cc2cc(NS(=O)(=O)Cc3ccc(F)cc3)cc3ccccc23)CC1] | (1-methylpiperidin-4-yl)- methyl 6-(aminomethyl)- 1-({3-[(4-fluorophenyl)- methanesulfonamido]- naphthalen-1-yl}meth- yl)-1H-pyrrolo[2,3-b]- pyridine-2-carboxylate | 630.4 | 100 | A |
| 207 | 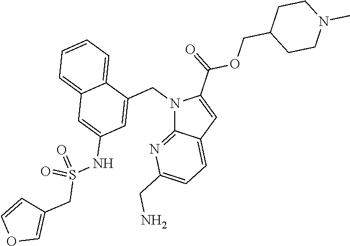 [SMILES: CN1CCC(COC(=O)c2cc3ccc(CN)nc3n2Cc2cc(NS(=O)(=O)Cc3ccoc3)cc3ccccc23)CC1] | (1-methylpiperidin-4-yl)- methyl 6-(aminomethyl)- 1-({3-[(furan-3-yl)meth- anesulfonamido]naph- thalen-1-yl}methyl)-1H- pyrrolo[2,3-b]pyridine- 2-carboxylate | 602.0 | 95 | A |
| 208 | 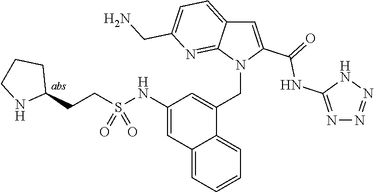 [SMILES: NCc1ccc2cc(C(=O)Nc3nnn[nH]3)n(Cc3cc(NS(=O)(=O)CC[C@@H]4CCCN4)cc4ccccc34)c2n1] | 6-(aminomethyl)-1-[(3- (2-[(2S)-pyrrolidin-2- yl]ethanesulfonamido}- naphthalen-1-yl)methyl]- N-(1H-1,2,3,4-tetrazol- 5-yl)-1H-pyrrolo[2,3-b]- pyridine-2-carboxamide | 575.3 | 99 | A |
| 209 | 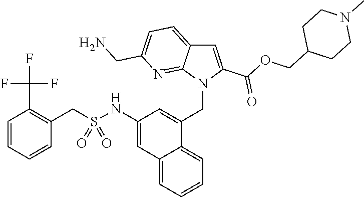 [SMILES: CN1CCC(COC(=O)c2cc3ccc(CN)nc3n2Cc2cc(NS(=O)(=O)Cc3ccccc3C(F)(F)F)cc3ccccc23)CC1] | (1-methylpiperidin-4-yl)- methyl 6-(aminomethyl)- 1-[(3-{[2-(trifluorometh- yl)phenyl]methanesul- fonamido}naphthalen-1- yl)methyl]-1H-pyrrolo- [2,3-b]pyridine-2-car- boxylate | 680.2 | 99 | A |
| 210 | 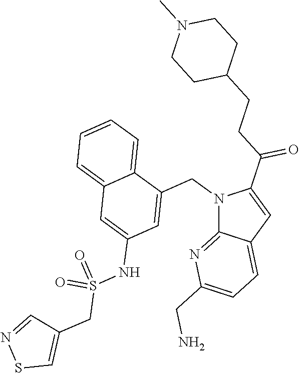 [SMILES: CN1CCC(CCC(=O)c2cc3ccc(CN)nc3n2Cc2cc(NS(=O)(=O)Cc3cnsc3)cc3ccccc23)CC1] | (1-methylpiperidin-4-yl)- methyl 6-(aminomethyl)- 1-({3-[(1,2-thiazol-4-yl)- methanesulfonamido]- naphthalen-1-yl}methyl)- 1H-pyrrolo[2,3-b]pyr- idine-2-carboxylate | 618.8 | 99 | A |
| 211 | 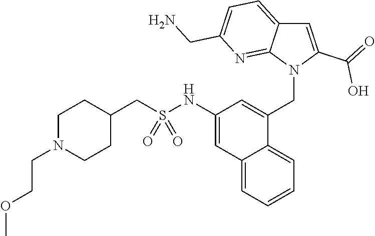 [SMILES: COCCN1CCC(CS(=O)(=O)Nc2cc(Cn3c(C(=O)O)cc4ccc(CN)nc43)c3ccccc3c2)CC1] | 6-(aminomethyl)-1-[(3- {[1-(2-methoxyethyl)- piperidin-4-yl]methane- sulfonamido}naphthalen- 1-yl)methyl]-1H-pyr- rolo[2,3-b]pyridine-2- carboxylic acid | 566.1 | 100 | A |
| 212 | 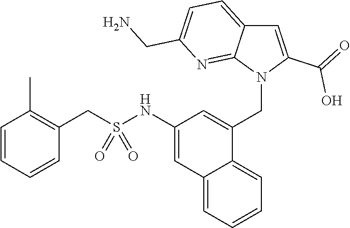 [SMILES: Cc1ccccc1CS(=O)(=O)Nc1cc(Cn2c(C(=O)O)cc3ccc(CN)nc32)c2ccccc2c1] | 6-(aminomethyl)-1-({3- [(2-methylphenyl)meth- anesulfonamido]naph- thalen-1-yl}methyl)-1H- pyrrolo[2,3-b]pyridine- 2-carboxylic acid | 515.1 | 95 | A |
| 213 | 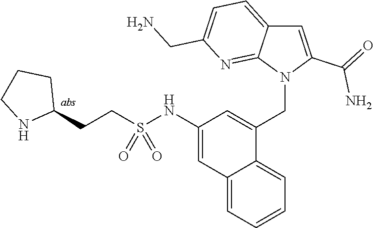 [SMILES: NCc1ccc2cc(C(N)=O)n(Cc3cc(NS(=O)(=O)CC[C@@H]4CCCN4)cc4ccccc34)c2n1] | 6-(aminomethyl)-1-[(3- {2-[(2S)-pyrrolidin-2- yl]ethanesulfonamido}- naphthalen-1-yl)methyl]- 1H-pyrrolo[2,3-b]pyr- idine-2-carboxamide | 507.3 | 100 | A |
| 214 | 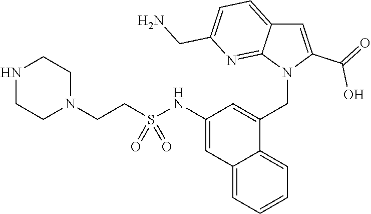 [SMILES: NCc1ccc2cc(C(=O)O)n(Cc3cc(NS(=O)(=O)CCN4CCNCC4)cc4ccccc34)c2n1] | 6-(aminomethyl)-1-({3- [2-(piperazin-1-yl)- ethanesulfonamido]- naphthalen-1-yl}meth- yl)-1H-pyrrolo[2,3-b]- pyridine-2-carboxylic acid | 523.3 | 100 | A |
| 215 | 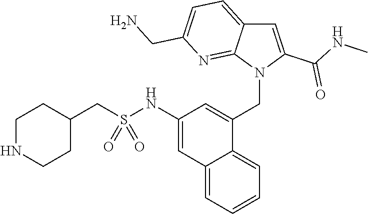 [SMILES: CNC(=O)c1cc2ccc(CN)nc2n1Cc1cc(NS(=O)(=O)CC2CCNCC2)cc2ccccc12] | 6-(aminomethyl)-N- methyl-1-({3-[(piperi- din-4-yl)methanesulfon- amido]naphthalen-1-yl}- methyl)-1H-pyrrolo[2,3- b]pyridine-2-carboxamide | 521.3 | 99 | B |
| 216 | 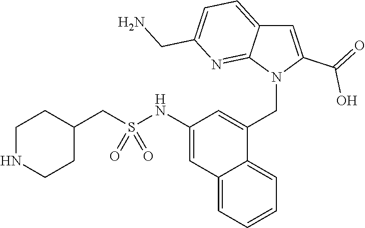 [SMILES: NCc1ccc2cc(C(=O)O)n(Cc3cc(NS(=O)(=O)CC4CCNCC4)cc4ccccc34)c2n1] | 6-(aminomethyl)-1-({3- [(piperidin-4-yl)methane- sulfonamido]naphthalen- 1-yl}methyl)-1H-pyr- rolo[2,3-b]pyridine-2- carboxylic acid | 508.4 | 98 | B |
| 217 | 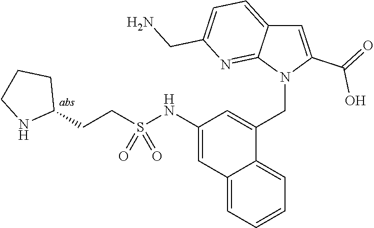 [SMILES: NCc1ccc2cc(C(=O)O)n(Cc3cc(NS(=O)(=O)CC[C@H]4CCCN4)cc4ccccc34)c2n1] | 6-(aminomethyl)-1-[(3- {2-[(2R)-pyrrolidin-2- yl]ethanesulfonamido}- naphthalen-1-yl)methyl]- 1H-pyrrolo[2,3-b]pyr- idine-2-carboxylic acid | 508.1 | 98 | B |
| 218 | 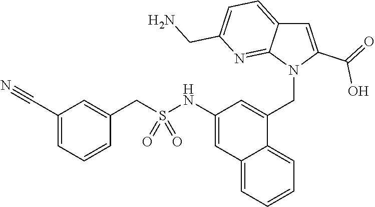 [SMILES: N#Cc1cccc(CS(=O)(=O)Nc2cc(Cn3c(C(=O)O)cc4ccc(CN)nc43)c3ccccc3c2)c1] | 6-(aminomethyl)-1-({3- [(3-cyanophenyl)meth- anesulfonamido]naph- thalen-1-yl}methyl)-1H- pyrrolo[2,3-b]pyridine- 2-carboxylic acid | 526.3 | 94 | B |
| 219 | 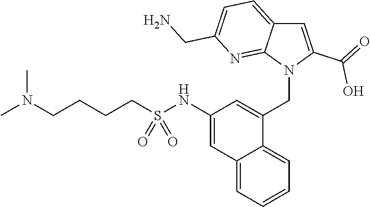 [SMILES: CN(C)CCCCS(=O)(=O)Nc1cc(Cn2c(C(=O)O)cc3ccc(CN)nc32)c2ccccc2c1] | 6-(aminomethyl)-1-({3- [4-(dimethylamino)- butanesulfonamido]naph- thalen-1-yl}methyl)-1H- pyrrolo[2,3-b]pyridine- 2-carboxylic acid | 510.0 | 94 | B |
| 220 | 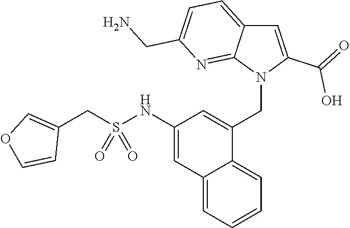 [SMILES: NCc1ccc2cc(C(=O)O)n(Cc3cc(NS(=O)(=O)Cc4ccoc4)cc4ccccc34)c2n1] | 6-(aminomethyl)-1-({3- [(furan-3-yl)methane- sulfonamido]naphthalen- 1-yl}methyl)-1H-pyr- rolo[2,3-b]pyridine-2- carboxylic acid | 491.3 | 100 | B |
| 221 | 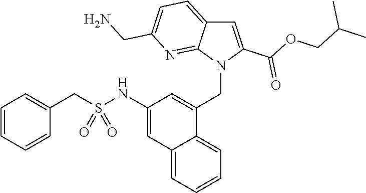 [SMILES: CC(C)COC(=O)c1cc2ccc(CN)nc2n1Cc1cc(NS(=O)(=O)Cc2ccccc2)cc2ccccc12] | (1-methylpiperidin-4- yl)methyl 6-(aminometh- yl)-1-({3-[(3-cyanophen- yl)methanesulfonamido]- naphthalen-1-yl}methyl)- 1H-pyrrolo[2,3-b]pyr- idine-2-carboxylate | 637.4 | 100 | B |
| 222 | 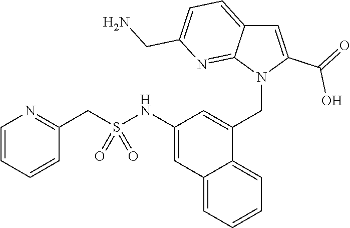 [SMILES: NCc1ccc2cc(C(=O)O)n(Cc3cc(NS(=O)(=O)Cc4ccccn4)cc4ccccc34)c2n1] | 6-(aminomethyl)-1-({3- [(pyridin-2-yl)methane- sulfonamido]naphthalen- 1-yl}methyl)-1H-pyr- rolo[2,3-b]pyridine-2- carboxylic acid | 502.1 | 99 | B |
| 223 | 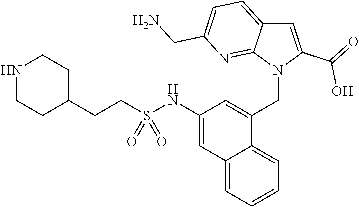 [SMILES: NCc1ccc2cc(C(=O)O)n(Cc3cc(NS(=O)(=O)CCC4CCNCC4)cc4ccccc34)c2n1] | 6-(aminomethyl)-1-({3- [2-(piperidin-4-yl)- ethanesulfonamido]- naphthalen-1-yl}meth- yl)-1H-pyrrolo[2,3-b]- pyridine-2-carboxylic acid | 522.3 | 96 | B |
| 224 | 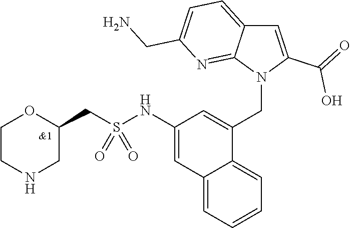 [SMILES: NCc1ccc2cc(C(=O)O)n(Cc3cc(NS(=O)(=O)C[C@H]4CNCCO4)cc4ccccc34)c2n1] | 6-(aminomethyl)-1-({3- [(morpholin-2-yl)- methanesulfonamido]- naphthalen-1-yl}meth- yl)-1H-pyrrolo[2,3-b]- pyridine-2-carboxylic acid | 510.5 | 98 | B |
| 225 | 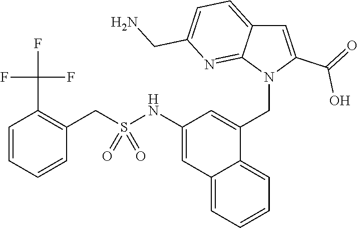 [SMILES: NCc1ccc2cc(C(=O)O)n(Cc3cc(NS(=O)(=O)Cc4ccccc4C(F)(F)F)cc4ccccc34)c2n1] | 6-(aminomethyl)-1-[(3- {[2-(trifluoromethyl)- phenyl]methanesulfon- amido}naphthalen-1- yl)methyl]-1H-pyrrolo- [2,3-b]pyridine-2-car- boxylic acid | 569.0 | 100 | B |
| 226 | 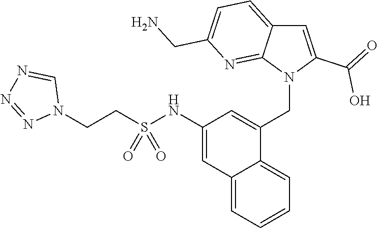 [SMILES: NCc1ccc2cc(C(=O)O)n(Cc3cc(NS(=O)(=O)CCn4cnnn4)cc4ccccc34)c2n1] | 6-(aminomethyl)-1-({3- [2-(1H-1,2,3,4-tetrazol- 1-yl)ethanesulfonamido]- naphthalen-1-yl}methyl)- 1H-pyrrolo[2,3-b]pyr- idine-2-carboxylic acid | 507.0 | 89 | B |
| 227 | 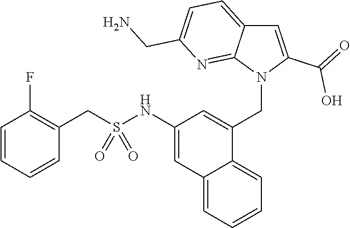 [SMILES: NCc1ccc2cc(C(=O)O)n(Cc3cc(NS(=O)(=O)Cc4ccccc4F)cc4ccccc34)c2n1] | 6-(aminomethyl)-1-({3- [(2-fluorophenyl)meth- anesulfonamido]naph- thalen-1-yl}methyl)-1H- pyrrolo[2,3-b]pyridine- 2-carboxylic acid | 519.0 | 99 | B |
| 228 | 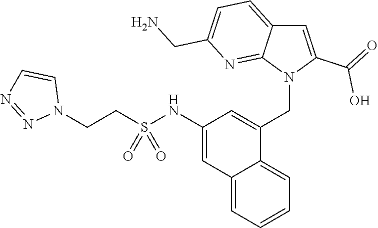 [SMILES: NCc1ccc2cc(C(=O)O)n(Cc3cc(NS(=O)(=O)CCn4ccnn4)cc4ccccc34)c2n1] | 6-(aminomethyl)-1-({3- [2-(1H-1,2,3-triazol-1- yl)ethanesulfonamido]- naphthalen-1-yl}meth- yl)-1H-pyrrolo[2,3-b]- pyridine-2-carboxylic acid | 506.0 | 99 | B |
| 229 | 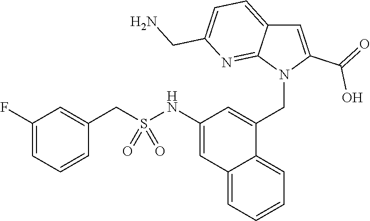 [SMILES: NCc1ccc2cc(C(=O)O)n(Cc3cc(NS(=O)(=O)Cc4cccc(F)c4)cc4ccccc34)c2n1] | 6-(aminomethyl)-1-({3- [(3-fluorophenyl)meth- anesulfonamido]naph- thalen-1-yl}methyl)-1H- pyrrolo[2,3-b]pyridine- 2-carboxylic acid | 519.0 | 99 | B |
| 230 | 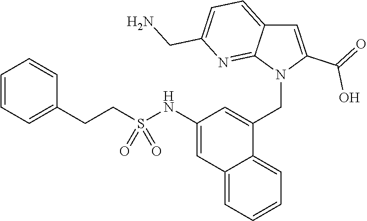 [SMILES: NCc1ccc2cc(C(=O)O)n(Cc3cc(NS(=O)(=O)CCc4ccccc4)cc4ccccc34)c2n1] | 6-(aminomethyl)-1-{[3- (2-phenylethanesulfon- amido)naphthalen-1-yl]- methyl}-1H-pyrrolo[2,3- b]pyridine-2-carboxylic acid | 515.2 | 99 | B |
| 231 | 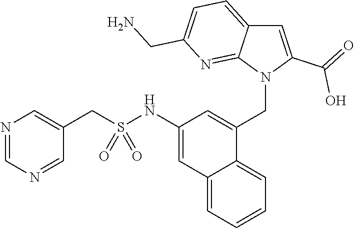 [SMILES: NCc1ccc2cc(C(=O)O)n(Cc3cc(NS(=O)(=O)Cc4cncnc4)cc4ccccc34)c2n1] | 6-(aminomethyl)-1-({3- [(pyrimidin-5-yl)methane- sulfonamido]naphthalen- 1-yl}methyl)-1H-pyr- rolo[2,3-b]pyridine-2- carboxylic acid | 503.0 | 99 | B |
| 232 | 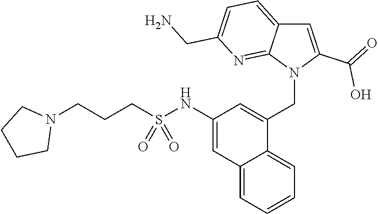 [SMILES: NCc1ccc2cc(C(=O)O)n(Cc3cc(NS(=O)(=O)CCCN4CCCC4)cc4ccccc34)c2n1] | 6-(aminomethyl)-1-({3- [3-(pyrrolidin-1-yl)- propanesulfonamido]- naphthalen-1-yl}methyl)- 1H-pyrrolo[2,3-b]pyr- idine-2-carboxylic acid | 522.0 | 92 | B |
| 233 | 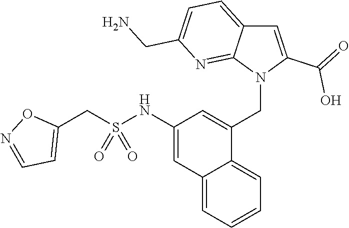 [SMILES: NCc1ccc2cc(C(=O)O)n(Cc3cc(NS(=O)(=O)Cc4ccno4)cc4ccccc34)c2n1] | 6-(aminomethyl)-1-({3- [(1,2-oxazol-5-yl)- methanesulfonamido]- naphthalen-1-yl}methyl)- 1H-pyrrolo[2,3-b]pyr- idine-2-carboxylic acid | 492.0 | 99 | B |
| 234 | 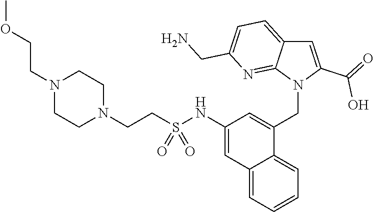 [SMILES: COCCN1CCN(CCS(=O)(=O)Nc2cc(Cn3c(C(=O)O)cc4ccc(CN)nc43)c3ccccc3c2)CC1] | 6-(aminomethyl)-1-[(3- {2-[4-(2-methoxyethyl)- piperazin-1-yl]ethane- sulfonamido}naphthalen- 1-yl)methyl]-1H-pyr- rolo[2,3-b]pyridine-2- carboxylic acid | 581.1 | 99 | B |
| 235 | 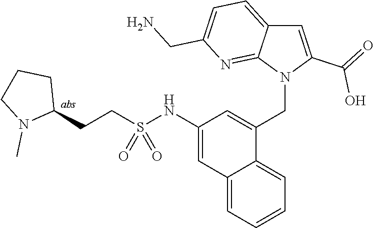 [SMILES: CN1CCC[C@H]1CCS(=O)(=O)Nc1cc(Cn2c(C(=O)O)cc3ccc(CN)nc32)c2ccccc2c1] | 6-(aminomethyl)-1-[(3- {2-[(2S)-1-methylpyr- rolidin-2-yl]ethanesul- fonamido}naphthalen-1- yl)methyl]-1H-pyrrolo- [2,3-b]pyridine-2-car- boxylic acid | 522.3 | 96 | B |
| 236 | 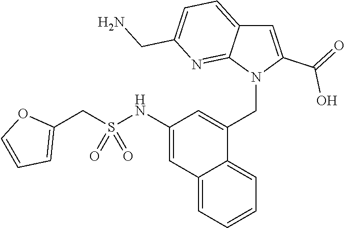 [SMILES: NCc1ccc2cc(C(=O)O)n(Cc3cc(NS(=O)(=O)Cc4ccco4)cc4ccccc34)c2n1] | 6-(aminomethyl)-1-({3- [(furan-2-yl)methane- sulfonamido]naphthalen- 1-yl}methyl)-1H-pyr- rolo[2,3-b]pyridine-2- carboxylic acid | 491.3 | 100 | B |
| 237 | 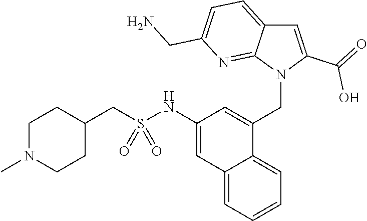 [SMILES: CN1CCC(CS(=O)(=O)Nc2cc(Cn3c(C(=O)O)cc4ccc(CN)nc43)c3ccccc3c2)CC1] | 6-(aminomethyl)-1-({3- [(1-methylpiperidin-4- yl)methanesulfonamido]- naphthalen-1-yl}methyl)- 1H-pyrrolo[2,3-b]pyr- idine-2-carboxylic acid | 522.1 | 99 | B |
| 238 | 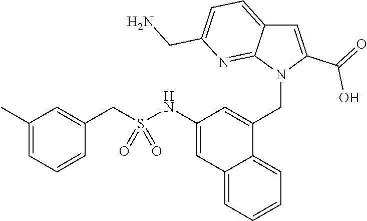 [SMILES: Cc1cccc(CS(=O)(=O)Nc2cc(Cn3c(C(=O)O)cc4ccc(CN)nc43)c3ccccc3c2)c1] | 6-(aminomethyl)-1-({3- [(3-methylphenyl)meth- anesulfonamido]naph- thalen-1-yl}methyl)-1H- pyrrolo[2,3-b]pyridine- 2-carboxylic acid | 515.1 | 97 | B |
| 239 | 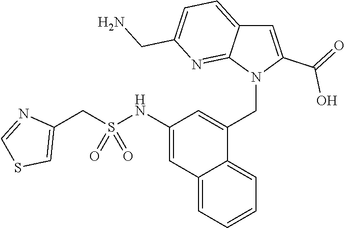 [SMILES: NCc1ccc2cc(C(=O)O)n(Cc3cc(NS(=O)(=O)Cc4cscn4)cc4ccccc34)c2n1] | 6-(aminomethyl)-1-({3- [(1,3-thiazol-4-yl)meth- anesulfonamido]naph- thalen-1-yl}methyl)-1H- pyrrolo[2,3-b]pyridine- 2-carboxylic acid | 508.0 | 100 | B |
| 240 | 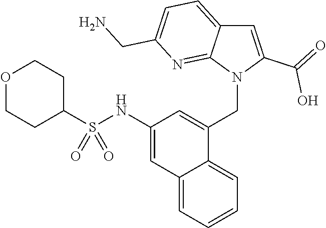 [SMILES: NCc1ccc2cc(C(=O)O)n(Cc3cc(NS(=O)(=O)C4CCOCC4)cc4ccccc34)c2n1] | 6-(aminomethyl)-1-{[3- (oxane-4-sulfonamido)- naphthalen-1-yl]methyl}- 1H-pyrrolo[2,3-b]pyr- idine-2-carboxylic acid | 495.0 | 98 | B |
| 241 | 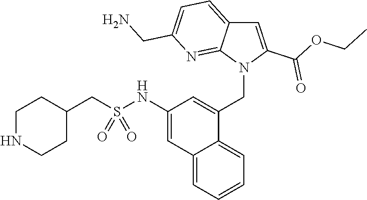 [SMILES: CCOC(=O)c1cc2ccc(CN)nc2n1Cc1cc(NS(=O)(=O)CC2CCNCC2)cc2ccccc12] | ethyl 6-(aminomethyl)- 1-({3-[(piperidin-4-yl)- methanesulfonamido]- naphthalen-1-yl}methyl)- 1H-pyrrolo[2,3-b]pyr- idine-2-carboxylate | 536.4 | 98 | B |
| 242 | 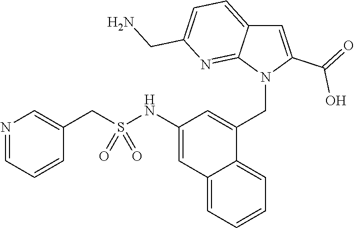 [SMILES: NCc1ccc2cc(C(=O)O)n(Cc3cc(NS(=O)(=O)Cc4cccnc4)cc4ccccc34)c2n1] | 6-(aminomethyl)-1-({3- [(pyridin-3-yl)methane- sulfonamido]naphthalen- 1-yl}methyl)-1H-pyr- rolo[2,3-b]pyridine-2- carboxylic acid | 502.0 | 96 | B |
| 243 | 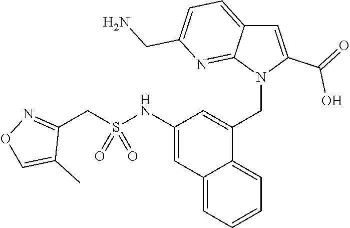 [SMILES: Cc1conc1CS(=O)(=O)Nc1cc(Cn2c(C(=O)O)cc3ccc(CN)nc32)c2ccccc2c1] | 6-(aminomethyl)-1-({3- [(4-methyl-1,2-oxazol-3- yl)methanesulfonamido]- naphthalen-1-yl}methyl)- 1H-pyrrolo[2,3-b]pyr- idine-2-carboxylic acid | 506.0 | 99 | B |
| 244 | 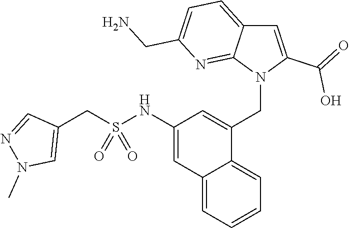 [SMILES: Cn1cc(CS(=O)(=O)Nc2cc(Cn3c(C(=O)O)cc4ccc(CN)nc43)c3ccccc3c2)cn1] | 6-(aminomethyl)-1-({3- [(1-methyl-1H-pyrazol-4- yl)methanesulfonamido]- naphthalen-1-yl}methyl)- 1H-pyrrolo[2,3-b]pyr- idine-2-carboxylic acid | 505.3 | 99 | B |
| 245 | 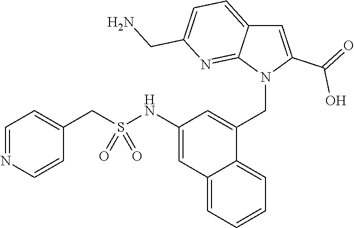 [SMILES: NCc1ccc2cc(C(=O)O)n(Cc3cc(NS(=O)(=O)Cc4ccncc4)cc4ccccc34)c2n1] | 6-(aminomethyl)-1-({3- [(pyridin-4-yl)methane- sulfonamido]naphthalen- 1-yl}methyl)-1H-pyr- rolo[2,3-b]pyridine-2- carboxylic acid | 502.3 | 97 | B |
| 246 | 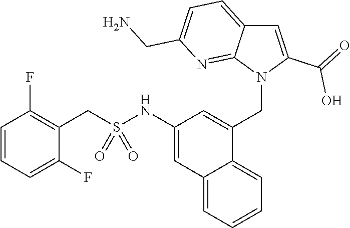 [SMILES: NCc1ccc2cc(C(=O)O)n(Cc3cc(NS(=O)(=O)Cc4c(F)cccc4F)cc4ccccc34)c2n1] | 6-(aminomethyl)-1-({3- [(2,6-difluorophenyl)- methanesulfonamido]- naphthalen-1-yl}meth- yl)-1H-pyrrolo[2,3-b]- pyridine-2-carboxylic acid | 537.2 | 99 | B |
| 247 | 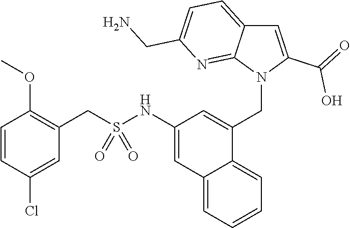 [SMILES: COc1ccc(Cl)cc1CS(=O)(=O)Nc1cc(Cn2c(C(=O)O)cc3ccc(CN)nc32)c2ccccc2c1] | 6-(aminomethyl)-1-({3- [(5-chloro-2-methoxy- phenyl)methanesulfon- amido]naphthalen-1-yl}- methyl)-1H-pyrrolo[2,3- b]pyridine-2-carboxylic acid | 566.3 | 98 | C |
| 248 | 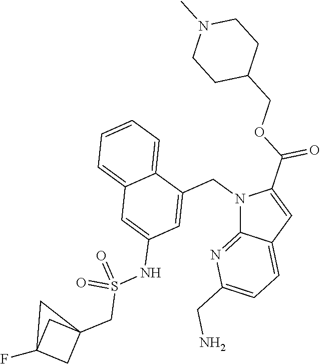 [SMILES: CN1CCC(COC(=O)c2cc3ccc(CN)nc3n2Cc2cc(NS(=O)(=O)CC34CC(F)(C3)C4)cc3ccccc23)CC1] | (1-methylpiperidin-4-yl)- methyl 6-(aminomethyl)- 1-{[3-({3-fluorobicyclo- [1.1.1]pentan-1-yl}- methanesulfonamido)- naphthalen-1-yl]methyl}- 1H-pyrrolo[2,3-b]pyr- idine-2-carboxylate | 620.5 | 98 | C |
| 249 | 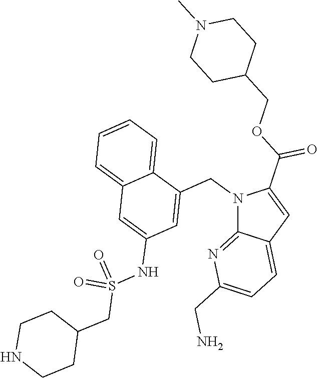 [SMILES: CN1CCC(COC(=O)c2cc3ccc(CN)nc3n2Cc2cc(NS(=O)(=O)CC3CCNCC3)cc3ccccc23)CC1] | (1-methylpiperidin-4- yl)methyl 6-(amino- methyl)-1-({3-{(piperi- din-4-yl)methanesul- fonamido]naphthalen-1- yl}methyl)-1H-pyr- rolo[2,3-b]pyridine-2- carboxylate | 619.1 | 99 | C |
| 250 | 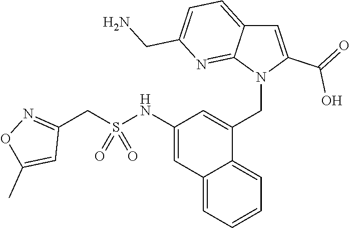 [SMILES: Cc1cc(CS(=O)(=O)Nc2cc(Cn3c(C(=O)O)cc4ccc(CN)nc43)c3ccccc3c2)no1] | 6-(aminomethyl)-1-({3- [(5-methyl-1,2-oxazol- 3-yl)methanesulfon- amido]naphthalen-1-yl}- methyl)-1H-pyrrolo[2,3- b]pyridine-2-carboxylic acid | 506.3 | 100 | C |
| 251 | 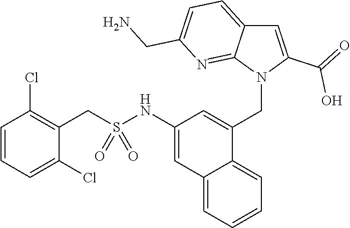 [SMILES: NCc1ccc2cc(C(=O)O)n(Cc3cc(NS(=O)(=O)Cc4c(Cl)cccc4Cl)cc4ccccc34)c2n1] | 6-(aminomethyl)-1-({3- [(2,6-dichlorophenyl)- methanesulfonamido]- naphthalen-1-yl}methyl)- 1H-pyrrolo[2,3-b]pyr- idine-2-carboxylic acid | 569.2 | 100 | C |
| 252 | 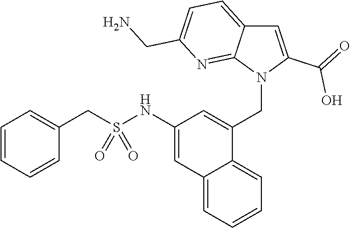 [SMILES: NCc1ccc2cc(C(=O)O)n(Cc3cc(NS(=O)(=O)Cc4ccccc4)cc4ccccc34)c2n1] | 6-(aminomethyl)-1-{[3- (phenylmethanesulfon- ylmethyl)naphthalen-1- yl]methyl}-1H-pyrrolo- [2,3-b]pyridine-2-car- boxylic acid | 500.3 | 100 | C |
| 253 | 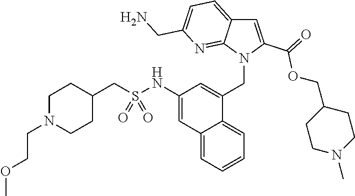 [SMILES: COCCN1CCC(CS(=O)(=O)Nc2cc(Cn3c(C(=O)OCC4CCN(C)CC4)cc4ccc(CN)nc43)c3ccccc3c2)CC1] | (1-methylpiperidin-4- yl)methyl 6-(amino- methyl)-1-1(3-{[1-(2- methoxyethyl)piperidin- 4-yl]methanesulfon]- amido}naphthalen-1-yl)- methyl]-1H-pyrrolo[2,3- b]pyridine-2-carboxylate | 677.5 | 100 | C |
| 254 | 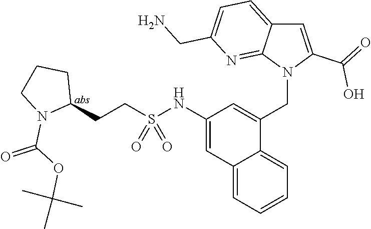 [SMILES: CC(C)(C)OC(=O)N1CCC[C@H]1CCS(=O)(=O)Nc1cc(Cn2c(C(=O)O)cc3ccc(CN)nc32)c2ccccc2c1] | 6-(aminomethyl)-1-[(3- {2-[(2S)-1-[(tert-butoxy)- carbonyl]pyrrolidin-2- yl]ethanesulfonamido}- naphthalen-1-yl)methyl]- 1H-pyrrolo[2,3-b]pyr- idine-2-carboxylic acid | 608.3 | 97 | |
| 255 | 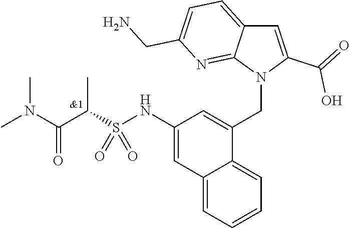 [SMILES: C[C@@H](C(=O)N(C)C)S(=O)(=O)Nc1cc(Cn2c(C(=O)O)cc3ccc(CN)nc32)c2ccccc2c1] | rac-6-(aminomethyl)-1- ({3-(dimethylcarbam- oyl)ethanesulfonamido]- naphthalen-1-yl}methyl)- 1H-pyrrolo[2,3-b]pyr- idine-2-carboxylic acid | 510.0 | 99 | A |
| 256 | 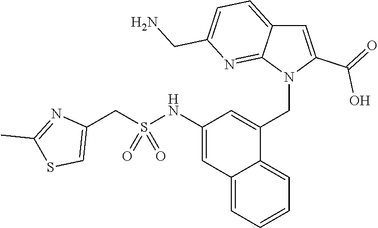 [SMILES: Cc1nc(CS(=O)(=O)Nc2cc(Cn3c(C(=O)O)cc4ccc(CN)nc43)c3ccccc3c2)cs1] | 6-(aminomethyl)-1-({3- [(2-methyl-1,3-thiazol- 4-yl)methanesulfon- amido]naphthalen-1-yl}- methyl)-1H-pyrrolo[2,3- b]pyridine-2-carboxylic acid | 522.0 | 100 | B |
| 257 | 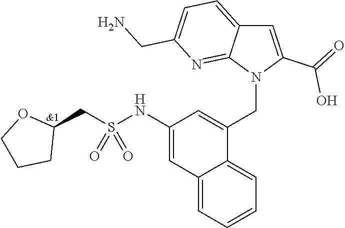 [SMILES: NCc1ccc2cc(C(=O)O)n(Cc3cc(NS(=O)(=O)C[C@H]4CCCO4)cc4ccccc34)c2n1] | rac-6-(aminomethyl)-1- [(3-{[(2R)-oxolan-2- yl]methanesulfonamido}- naphthalen-1-yl)methyl]- 1H-pyrrolo[2,3-b]pyr- idine-2-carboxylic acid | 495.0 | 98 | B |
| 258 | 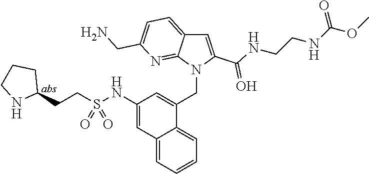 [SMILES: COC(=O)NCCNC(=O)c1cc2ccc(CN)nc2n1Cc1cc(NS(=O)(=O)CC[C@@H]2CCCN2)cc2ccccc12] | methyl N-(2-{[6-(amino- methyl)-1-[(3-{2-[(2S)- pyrrolidin-2-yl]ethane- sulfonamido}naphthalen- 1-yl)methyl]-1H-pyr- rolo[2,3-b]pyridin-2-yl]- formamido}ethyl)car- bamate | 608.4 | 98 | B |
| 259 | 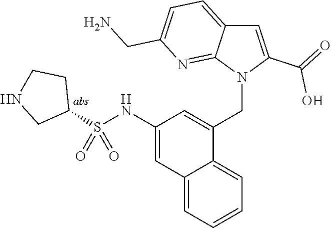 [SMILES: NCc1ccc2cc(C(=O)O)n(Cc3cc(NS(=O)(=O)[C@H]4CCNC4)cc4ccccc34)c2n1] | 6-(aminomethyl)-1-({3- [(3S)-pyrrolidine-3-sul- fonamido]naphthalen-1- yl}methyl)-1H-pyrrolo- [2,3-b]pyridine-2-car- boxylic acid | 480.3 | 100 | B |
| 260 | 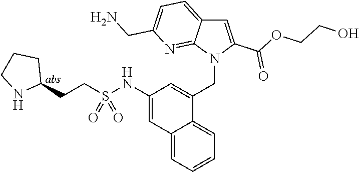 [SMILES: NCc1ccc2cc(C(=O)OCCO)n(Cc3cc(NS(=O)(=O)CC[C@@H]4CCCN4)cc4ccccc34)c2n1] | 2-hydroxyethyl 6- (aminomethyl)-1-[(3-{2- [(2S)-pyrrolidin-2-yl]- ethanesulfonamido}naph- thalen-1-yl)methyl]-1H- pyrrolo[2,3-b]pyridine- 2-carboxylate | 552.1 | 100 | B |
| 261 | 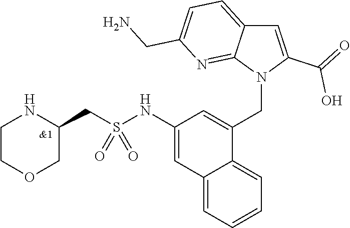 [SMILES: NCc1ccc2cc(C(=O)O)n(Cc3cc(NS(=O)(=O)C[C@H]4COCCN4)cc4ccccc34)c2n1] | 6-(aminomethyl)-1-({3- [(morpholin-3-l)methane- sulfonamido]naphthalen- 1-yl}methyl)-1H-pyr- rolo[2,3-b]pyridine-2- carboxylic acid | 510.2 | 99 | B |
| 262 | 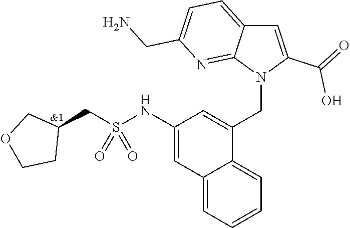 [SMILES: NCc1ccc2cc(C(=O)O)n(Cc3cc(NS(=O)(=O)C[C@H]4CCOC4)cc4ccccc34)c2n1] | rac-6-(aminomethyl)-1- [(3-{[(3R)-oxolan-3-yl]- methanesulfonamido}- naphthalen-1-yl)methyl]- 1H-pyrrolo[2,3-b]pyr- idine-2-carboxylic acid | 495.0 | 96 | B |
| 263 | 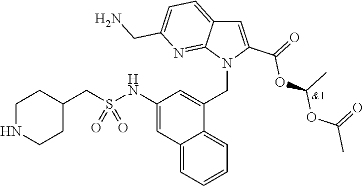 [SMILES: CC(=O)O[C@H](C)OC(=O)c1cc2ccc(CN)nc2n1Cc1cc(NS(=O)(=O)CC2CCNCC2)cc2ccccc12] | 1-[6-(aminomethyl)-1- ({3-[(piperidin-4-yl)- methanesulfonamido]- naphthlen-1-yl}methyl)- 1H-pyrrolo[2,3-b]pyr- idine-2-carbonyloxy]- ethyl acetate | 594.1 | 97 | B |
| 264 | 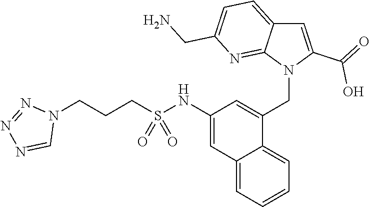 [SMILES: NCc1ccc2cc(C(=O)O)n(Cc3cc(NS(=O)(=O)CCCn4cnnn4)cc4ccccc34)c2n1] | 6-(aminomethyl)-1-({3- [3-(1H-1,2,3,4-tetrazol-1- yl)propanesulfonamido]- naphthalen-1-yl}methyl)- 1H-pyrrolo[2,3-b]pyr- idine-2-carboxylic acid | 521.0 | 100 | B |
| 265 | 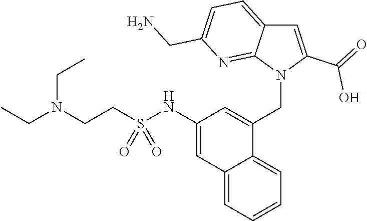 [SMILES: CCN(CC)CCS(=O)(=O)Nc1cc(Cn2c(C(=O)O)cc3ccc(CN)nc32)c2ccccc2c1] | 6-(aminomethyl)-1-({3- [2-(diethylamino)ethane- sulfonamido]naphthalen- 1-yl}methyl)-1H-pyr- rolo[2,3-b]pyridine-2- carboxylic acid | 510.0 | 95 | B |
| 266 | 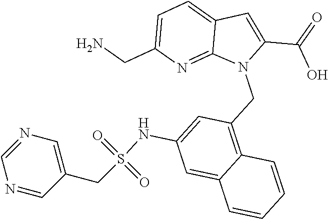 [SMILES: NCc1ccc2cc(C(=O)O)n(Cc3cc(NS(=O)(=O)Cc4cncnc4)cc4ccccc34)c2n1] | 6-(aminomethyl)-1-((3- ((pyrimidin-5-ylmeth- yl)sulfonamido)naph- thalen-1-yl)methyl)-1H- pyrrolo[2,3-b]pyridine- 2-carboxylic acid | 503.0 | 99.12 | B |
| 267 | 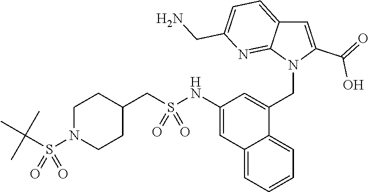 [SMILES: CC(C)(C)S(=O)(=O)N1CCC(CS(=O)(=O)Nc2cc(Cn3c(C(=O)O)cc4ccc(CN)nc43)c3ccccc3c2)CC1] | 6-(aminomethyl)-1-[(3- {[1-(2-methylpropane-2- sulfonyl)piperidin-4-yl]- methanesulfonamido}- naphthalen-1-yl)methyl]- 1H-pyrrolo[2,3-b]pyr- idine-2-carboxylic acid | 628.4 | 100 | B |
| 268 | 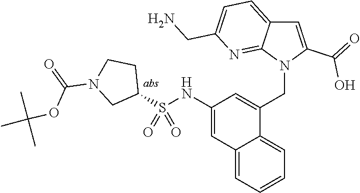 [SMILES: CC(C)(C)OC(=O)N1CC[C@H](S(=O)(=O)Nc2cc(Cn3c(C(=O)O)cc4ccc(CN)nc43)c3ccccc3c2)C1] | 6-(aminomethyl)-1-({3- [(3S)-1-[(tert-butoxy)- carbonyl]pyrrolidine-3- sulfonamido]naphthalen- 1-yl}methyl)-1H-pyr- rolo[2,3-blpyridine-2- carboxylic acid | 580.4 | 99 | B |
| 269 | 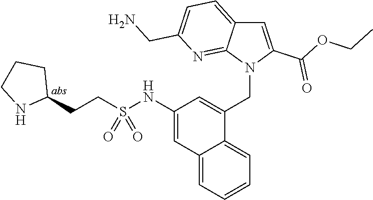 [SMILES: CCOC(=O)c1cc2ccc(CN)nc2n1Cc1cc(NS(=O)(=O)CC[C@@H]2CCCN2)cc2ccccc12] | ethyl 6-(aminomethyl)-1- [(3-{2-[(2S)-pyrrolidin-2- yl]ethanesulfonamido}- naphthalen-1-yl)methyl]- 1H-pyrrolo[2,3-b]pyr- idine-2-carboxylate | 535.6 | 92 | C |
| 270 | 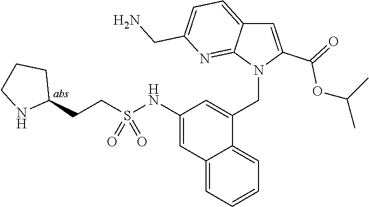 [SMILES: CC(C)OC(=O)c1cc2ccc(CN)nc2n1Cc1cc(NS(=O)(=O)CC[C@@H]2CCCN2)cc2ccccc12] | propan-2-yl 6-(amino- methyl)-1-[(3-{2-[(2S)- pyrrolidin-2-yl]ethane- sulfonamido}naphthalen- 1-yl)methyl]-1H-pyrrolo- [2,3-b]pyridine-2-car- boxylate | 550.4 | 86 | C |
| 271 | 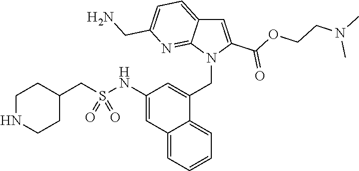 [SMILES: CN(C)CCOC(=O)c1cc2ccc(CN)nc2n1Cc1cc(NS(=O)(=O)CC2CCNCC2)cc2ccccc12] | 2-(dimethylamino)ethyl 6-(aminomethyl)-1-({3- [(piperidin-4-yl)meth- anesulfonamido]naph- thalen-1-yl}methyl)-1H- pyrrolo[2,3-b]pyridine- 2-carboxylate | 579.2 | 97 | C |
| 272 | 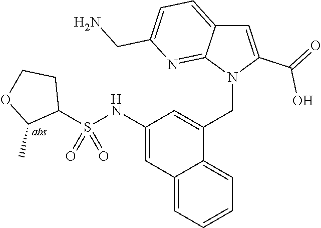 [SMILES: C[C@@H]1OCCC1S(=O)(=O)Nc1cc(Cn2c(C(=O)O)cc3ccc(CN)nc32)c2ccccc2c1] | 6-(aminomethyl)-1-({3- [(2S)-2-methyloxolane- 3-sulfonamido]naph- thalen-1-yl}methyl)-1H- pyrrolo[2,3-b]pyridine- 2-carboxylic acid | 495.0 | 99 | C |
| 273 | 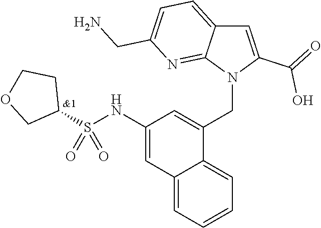 [SMILES: NCc1ccc2cc(C(=O)O)n(Cc3cc(NS(=O)(=O)[C@H]4CCOC4)cc4ccccc34)c2n1] | rac-6-(aminomethyl)-1- ({3-[(3R)-oxolane-3- sulfonamido]naphthalen- 1-yl}methyl)-1H-pyr- rolo[2,3-b]pyridine-2- carboxylic acid | 481.0 | 92 | C |
| 274 | 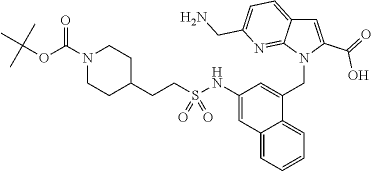 [SMILES: CC(C)(C)OC(=O)N1CCC(CCS(=O)(=O)Nc2cc(Cn3c(C(=O)O)cc4ccc(CN)nc43)c3ccccc3c2)CC1] | 6-(aminomethyl)-1-{[3- (2-{1-[(tert-butoxy)car- bonyl]piperidin-4-yl}- ethanesulfonamido)naph- thalen-1-yl]methyl}-1H- pyrrolo[2,3-b]pyridine- 2-carboxylic acid | 622.5 | 99 | C |
| 275 | 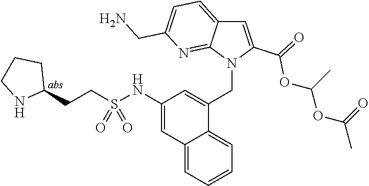 [SMILES: CC(=O)OC(C)OC(=O)c1cc2ccc(CN)nc2n1Cc1cc(NS(=O)(=O)CC[C@@H]2CCCN2)cc2ccccc12] | 1-[6-(aminomethyl)-1- [(3-{2-[(2S)-pyrrolidin- 2-yl]ethanesulfonamido} naphthalen-1-yl)methyl]- 1H-pyrrolo[2,3-b]pyr- idine-2-carbonyloxy]- ethyl acetate | 594.1 | 99 | C |
| 276 | 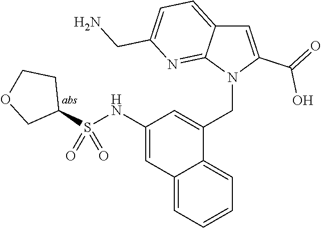 [SMILES: NCc1ccc2cc(C(=O)O)n(Cc3cc(NS(=O)(=O)[C@@H]4CCOC4)cc4ccccc34)c2n1] | 6-(aminomethyl)-1-({3- [(3R)-oxolane-3-sulfon- amido]naphthalen-1-yl}- methyl)-1H-pyrrolo[2,3- blpyridine-2-carboxylic acid | 481.0 | 97 | C |
| 277 | 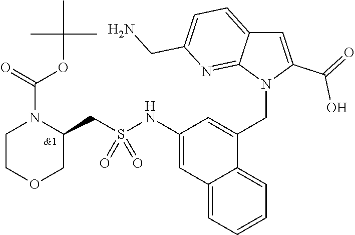 [SMILES: CC(C)(C)OC(=O)N1CCOC[C@@H]1CS(=O)(=O)Nc1cc(Cn2c(C(=O)O)cc3ccc(CN)nc32)c2ccccc2c1] | 6-(aminomethyl)-1-{[3- ({4-[(tert-butoxy)car- bonyl]morpholin-3-yl}- methanesulfonamido)- naphthalen-1-yl]methyl}- 1H-pyrrolo[2,3-b]pyr- idine-2-carboxylic acid | 610.1 | 96 | C |
| 278 | 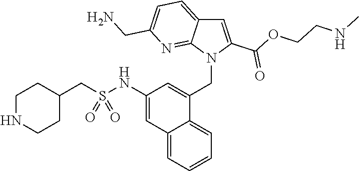 [SMILES: CNCCOC(=O)c1cc2ccc(CN)nc2n1Cc1cc(NS(=O)(=O)CC2CCNCC2)cc2ccccc12] | 2-(methylamino)ethyl 6- (aminomethyl)-1-({3- [(piperidin-4-yl)methane- sulfonamido]naphthalen- 1-yl}methyl)-1H-pyr- rolo[2,3-b]pyridine-2- carboxylate | 565.4 | 100 | C |
| 279 | 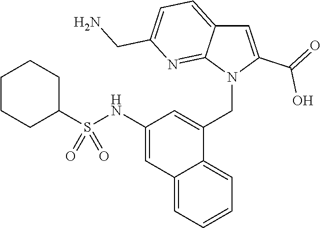 [SMILES: NCc1ccc2cc(C(=O)O)n(Cc3cc(NS(=O)(=O)C4CCCCC4)cc4ccccc34)c2n1] | 6-(aminomethyl)-1-[(3- cyclohexanesulfon- amidonaphthalen-1-yl)- methyl]-1H-pyrrolo[2,3- b]pyridine-2-carboxylic acid | 493.0 | 99 | C |
| 280 | 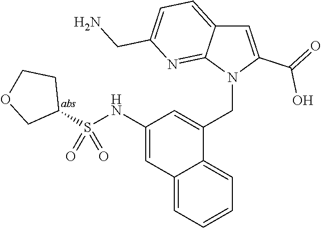 [SMILES: NCc1ccc2cc(C(=O)O)n(Cc3cc(NS(=O)(=O)[C@H]4CCOC4)cc4ccccc34)c2n1] | 6-(aminomethyl)-1-({3- [(3S)-oxolane-3-sulfon- amido]naphthalen-1-yl}- methyl)-1H-pyrrolo[2,3- b]pyridine-2-carboxylic acid | 481.0 | 94 | C |
| 281 | 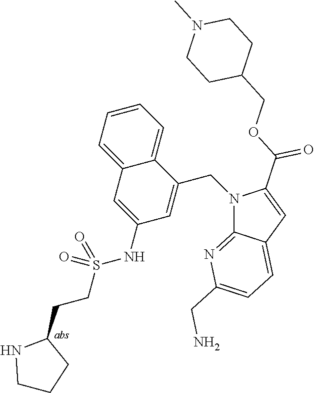 [SMILES: CN1CCC(COC(=O)c2cc3ccc(CN)nc3n2Cc2cc(NS(=O)(=O)CC[C@H]3CCCN3)cc3ccccc23)CC1] | (1-methylpiperidin-4- yl)methyl 6-(aminometh- yl)-1-[(3-{2-[(2R)-pyr- rolidin-2-yl]ethanesul- fonamido}naphthalen-1- yl)methyl]-1H-pyrrolo- [2,3-b]pyridine-2-car- boxylate | 619.4 | 98 | C |
| 282 | 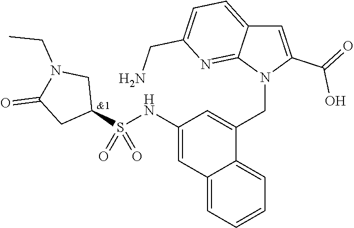 [SMILES: CCN1C[C@@H](S(=O)(=O)Nc2cc(Cn3c(C(=O)O)cc4ccc(CN)nc43)c3ccccc3c2)CC1=O] | rac-6-(aminomethyl)-1- ({3-[(3R)-1-ethyl-5- oxopyrrolidine-3-sulfon- amido]naphthalen-1-yl}- methyl)-1H-pyrrolo[2,3- b]pyridine-2-carboxylic acid | 522.0 | 94 | C |
| 283 | 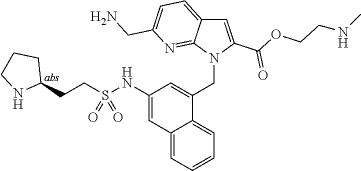 [SMILES: CNCCOC(=O)c1cc2ccc(CN)nc2n1Cc1cc(NS(=O)(=O)CC[C@@H]2CCCN2)cc2ccccc12] | 2-(methylamino)ethyl 6- (aminomethyl)-1-[(3-{2- [(2S)-pyrrolidin-2-yl]- ethanesulfonamido}- naphthalen-1-yl)methyl]- 1H-pyrrolo[2,3-b]pyr- idine-2-carboxylate | 565.1 | 94 | C |
| 284 | 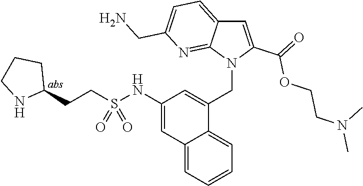 [SMILES: CN(C)CCOC(=O)c1cc2ccc(CN)nc2n1Cc1cc(NS(=O)(=O)CC[C@@H]2CCCN2)cc2ccccc12] | 2-(dimethylamino)ethyl 6-(aminomethyl)-1-[(3- {2-[(2S)-pyrrolidin-2- yl]ethanesulfonamido}- naphthalen-1-yl)methyl]- 1H-pyrrolo[2,3-b]pyr- idine-2-carboxylate | 579.4 | 95 | C |
| 285 | 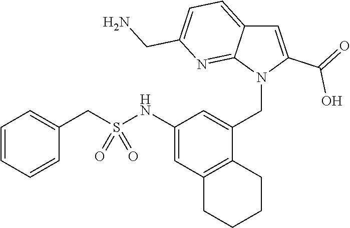 [SMILES: NCc1ccc2cc(C(=O)O)n(Cc3cc(NS(=O)(=O)Cc4ccccc4)cc4c3CCCC4)c2n1] | 6-(aminomethyl)-1-{[3- (phenylmethanesulfon- amido)-5,6,7,8-tetra- hydronaphthalen-1-yl]- methyl}-1H-pyrrolo[2,3- b]pyridine-2-carboxylic acid | 505.3 | 100 | B |
| 286 | 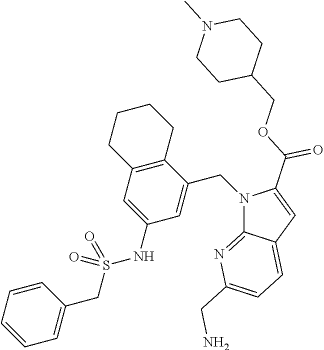 [SMILES: CN1CCC(COC(=O)c2cc3ccc(CN)nc3n2Cc2cc(NS(=O)(=O)Cc3ccccc3)cc3c2CCCC3)CC1] | (1-methylpiperidin-4-yl)- methyl 6-(aminomethyl)- 1-{[3-(phenylmethane- sulfonamido)-5,6,7,8- tetrahydronaphthalen-1- yl]methyl}-1H-pyrrolo- [2,3-b]pyridine-2-carbox- ylate | 616.5 | 98 | A |
| 287 | 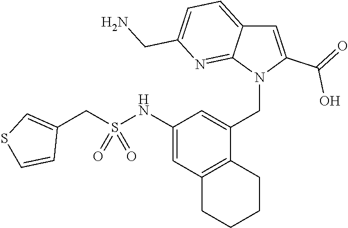 [SMILES: NCc1ccc2cc(C(=O)O)n(Cc3cc(NS(=O)(=O)Cc4ccsc4)cc4c3CCCC4)c2n1] | 6-(aminomethyl)-1-({3- [(thiophen-3-yl)methane- sulfonamido]-5,6,7,8- tetrahydronaphthalen-1- yl}methyl)-1H-pyrrolo- [2,3-b]pyridine-2-carbox- ylic acid | 511.3 | 98 | B |
| 288 | 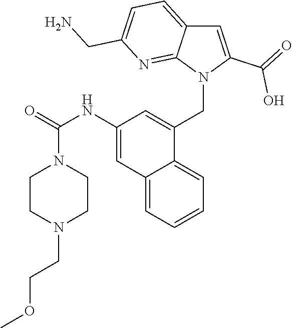 [SMILES: COCCN1CCN(C(=O)Nc2cc(Cn3c(C(=O)O)cc4ccc(CN)nc43)c3ccccc3c2)CC1] | 6-(aminomethyl)-1-[(3- {[4-(2-methoxyethyl)- piperazine-1-carbonyl]- amino}naphthalen-1-yl)- methyl]-1H-pyrrolo[2,3- b]pyridine-2-carboxylic acid | 517.3 | 99 | B |
| 289 | 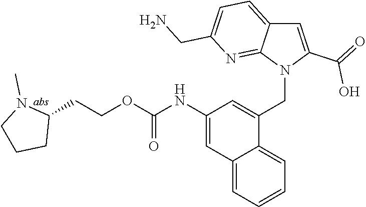 [SMILES: CN1CCC[C@H]1CCOC(=O)Nc1cc(Cn2c(C(=O)O)cc3ccc(CN)nc32)c2ccccc2c1] | 6-(aminomethyl)-1-({3- [({2-[(2S)-1-methyl- pyrrolidin-2-yl]ethoxy}- carbonyl)amino]naph- thalen-1-yl}methyl)-1H- pyrrolo[2,3-b]pyridine- 2-carboxylic acid | 502.4 | 98 | B |
| 290 | 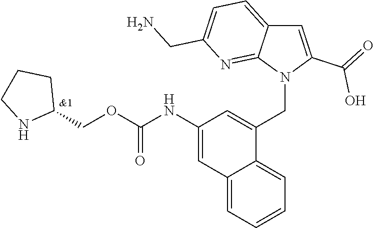 [SMILES: NCc1ccc2cc(C(=O)O)n(Cc3cc(NC(=O)OC[C@H]4CCCN4)cc4ccccc34)c2n1] | 6-(aminomethyl)-1-{[3- ({[(pyrrolidin-2-yl)meth- oxy]carbonyl}amino)- naphthalen-1-yl]methyl}- 1H-pyrrolo[2,3-b]pyr- idine-2-carboxylic acid | 474.3 | 98 | B |
| 291 | 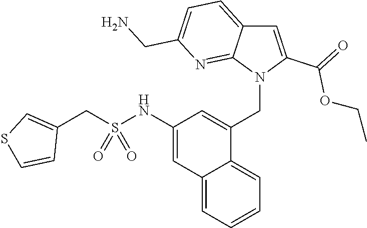 [SMILES: CCOC(=O)c1cc2ccc(CN)nc2n1Cc1cc(NS(=O)(=O)Cc2ccsc2)cc2ccccc12] | ethyl 6-(aminomethyl)- 1-({3-[(thiophen-3-yl)- methanesulfonamido]- naphthalen-1-yl}methyl)- 1H-pyrrolo[2,3-b]pyr- idine-2-carboxylate | 535.0 | 99 | B |
| 292 | 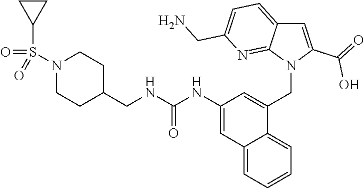 [SMILES: NCc1ccc2cc(C(=O)O)n(Cc3cc(NC(=O)NCC4CCN(S(=O)(=O)C5CC5)CC4)cc4ccccc34)c2n1] | 6-(aminomethyl)-1-({3- [({[1-(cyclopropanesul- fonyl)piperidin-4-yl]- methyl}carbamoyl)- amino]naphthalen-1-yl}- methyl)-1H-pyrrolo[2,3- b]pyridine-2-carboxylic acid | 591.4 | 97 | B |
| 293 | 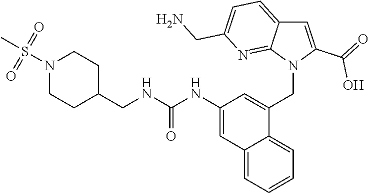 [SMILES: CS(=O)(=O)N1CCC(CNC(=O)Nc2cc(Cn3c(C(=O)O)cc4ccc(CN)nc43)c3ccccc3c2)CC1] | 6-(aminomethyl)-1-{[3- ({[(1-methanesulfonyl- piperidin-4-yl)methyl]- carbamoyl}amino)naph- thalen-1-yl]methyl}-1H- pyrrolo[2,3-b]pyridine- 2-carboxylic acid | 565.4 | 95 | B |
| 294 | 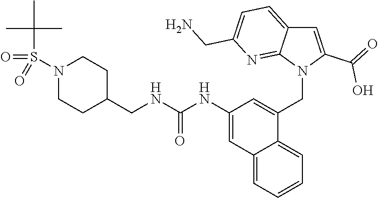 [SMILES: CC(C)(C)S(=O)(=O)N1CCC(CNC(=O)Nc2cc(Cn3c(C(=O)O)cc4ccc(CN)nc43)c3ccccc3c2)CC1] | 6-(aminomethyl)-1-({3- [({[1-(2-methylpropane- 2-sulfonyl)piperidin-4- yl]methyl}carbamoyl)- amino]naphthalen-1-yl}- methyl)-1H-pyrrolo[2,3- b]pyridine-2-carboxylic acid | 607.4 | 100 | B |
| 295 | 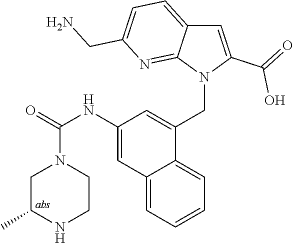 [SMILES: C[C@@H]1CN(C(=O)Nc2cc(Cn3c(C(=O)O)cc4ccc(CN)nc43)c3ccccc3c2)CCN1] | 6-(aminomethyl)-1-[(3- {[(3R)-3-methylpipera- zine-1-carbonyl]amino}- naphthalen-1-yl)methyl]- 1H-pyrrolo[2,3-b]pyr- idine-2-carboxylic acid | 473.0 | 93 | C |
| 296 | 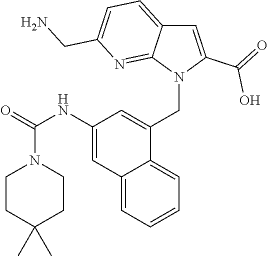 [SMILES: CC1(C)CCN(C(=O)Nc2cc(Cn3c(C(=O)O)cc4ccc(CN)nc43)c3ccccc3c2)CC1] | 6-(aminomethyl)-1-({3- [(4,4-dimethylpiperidine- 1-carbonyl)amino]naph- thalen-1-yl}methyl)-1H- pyrrolo[2,3-b]pyridine- 2-carboxylic acid | 486.0 | 98 | C |
| 297 | 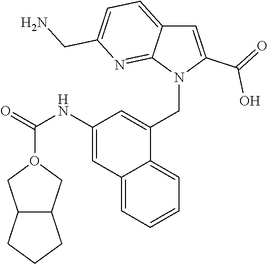 | 6-(aminomethyl)-1-{[3- ({octahydrocyclopenta- [c]pyrrole-2-carbonyl}- amino)naphthalen-1-yl]- methyl}-1H-pyrrolo[2,3- b]pyridine-2-carboxylic acid | 484.0 | 94 | C |
| 298 | 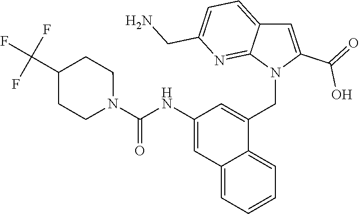 [SMILES: NCc1ccc2cc(C(=O)O)n(Cc3cc(NC(=O)N4CCC(C(F)(F)F)CC4)cc4ccccc34)c2n1] | 6-(aminomethyl)-1-[(3- {[4-(trifluoromethyl)- piperidine-1-carbonyl]- amino}naphthalen-1-yl)- methyl]-1H-pyrrolo[2,3- b]pyridine-2-carboxylic acid | 526.1 | 94 | C |
| 299 | 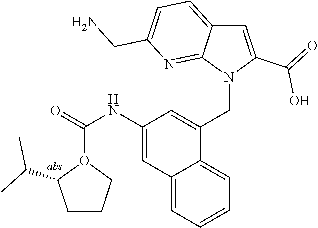 | 6-(aminomethyl)-1-[(3- {[(2R)-2-(propan-2-yl)- pyrrolidine-1-carbonyl]- amino}naphthalen-1-yl)- methyl]-1H-pyrrolo[2,3- b]pyridine-2-carboxylic acid | 486.0 | 100 | C |
| 300 | 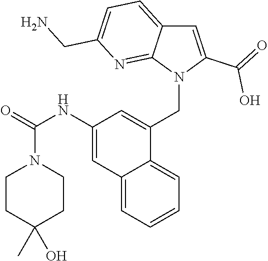 [SMILES: CC1(O)CCN(C(=O)Nc2cc(Cn3c(C(=O)O)cc4ccc(CN)nc43)c3ccccc3c2)CC1] | 6-(aminomethyl)-1-({3- [(4-hydroxy-4-methyl- piperidine-1-carbonyl)- aminoJnaphthalen-1-yl}- methyl)-1H-pyrrolo[2,3- b]pyridine-2-carboxylic acid | 488.0 | 96 | C |
| 301 | 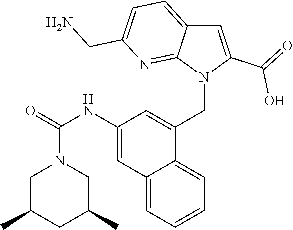 [SMILES: C[C@@H]1C[C@H](C)CN(C(=O)Nc2cc(Cn3c(C(=O)O)cc4ccc(CN)nc43)c3ccccc3c2)C1] | 6-(aminomethyl)-1-[(3- {[(3R,5S)-3,5-dimeth- ylpiperidine-1-carbonyl]- amino}naphthalen-1-yl)- methyl]-1H-pyrrolo[2,3- b]pyridine-2-carboxylic acid | 486.0 | 100 | C |
| 302 | 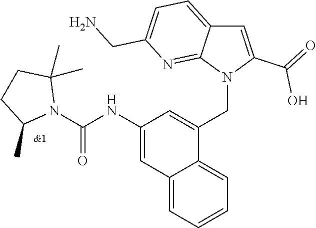 [SMILES: C[C@H]1CCC(C)(C)N1C(=O)Nc1cc(Cn2c(C(=O)O)cc3ccc(CN)nc32)c2ccccc2c1] | rac-6-(aminomethyl)-1- [(3-{[(SR)-2,2,5-trimeth- ylpyrrolidine-1-carbon- yl]amino}naphthalen-1- yl)methyl]-1H-pyrrolo- [2,3-b]pyridine-2-carbox- ylic acid | 486.0 | 93 | C |
| 303 | 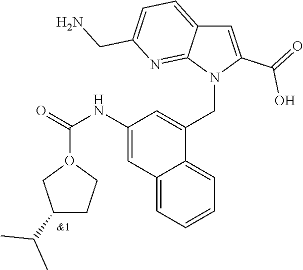 | rac-6-(aminomethyl)-1- [(3-{[(3R)-3-(propan-2- yl)pyrrolidine-1-carbon- yl]amino}naphthalen-1- yl)methyl]-1H-pyrrolo- [2,3-b]pyridine-2-car- boxylic acid | 486.0 | 95 | C |
| 304 | 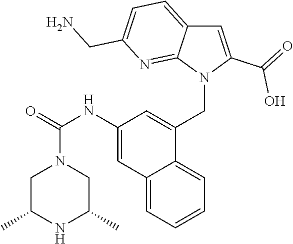 [SMILES: C[C@@H]1CN(C(=O)Nc2cc(Cn3c(C(=O)O)cc4ccc(CN)nc43)c3ccccc3c2)C[C@H](C)N1] | 6-(aminomethyl)-1-[(3- {[(3R,5S)-3,5-dimeth- ylpiperazine-1-carbon- yl]amino}naphthalen-1- yl)methyl]-1H-pyrrolo- [2,3-b]pyridine-2-car- boxylic acid | 487.0 | 94 | C |
| 305 | 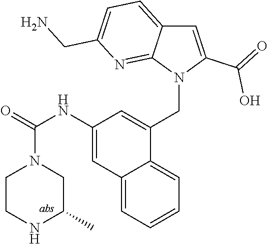 [SMILES: C[C@H]1CN(C(=O)Nc2cc(Cn3c(C(=O)O)cc4ccc(CN)nc43)c3ccccc3c2)CCN1] | 6-(aminomethyl)-1-1(3- {[(3S)-3-methylpipera- zine-1-carbonyl]amino}- naphthalen-1-yl)methyl]- 1H-pyrrolo[2,3-b]pyr- idine-2-carboxylic acid | 473.0 | 97 | C |
| 306 | 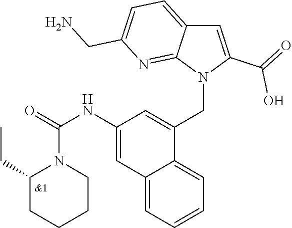 [SMILES: CC[C@H]1CCCCN1C(=O)Nc1cc(Cn2c(C(=O)O)cc3ccc(CN)nc32)c2ccccc2c1] | rac-6-(aminomethyl)-1- [(3-{[(2R)-2-ethylpiper- idine-1-carbonyl]amino}- naphthalen-1-yl)methyl]- 1H-pyrrolo[2,3-b]pyr- idine-2-carboxylic acid | 486.0 | 88 | C |
| 307 | 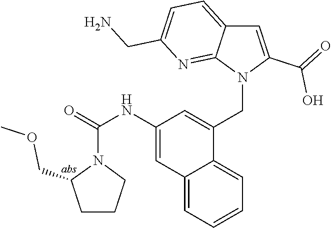 [SMILES: COC[C@H]1CCCN1C(=O)Nc1cc(Cn2c(C(=O)O)cc3ccc(CN)nc32)c2ccccc2c1] | 6-(aminomethyl)-1-1(3- {[(2R)-2-(methoxymeth- yl)pyrrolidine-1-carbon- yl]amino}naphthalen-1- yl)methyl]-1H-pyrrolo- [2,3-b]pyridine-2-car- boxylic acid | 488.0 | 100 | C |
| 308 | 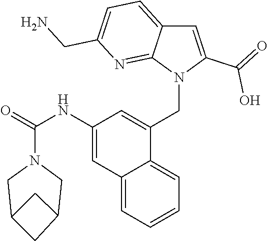 [SMILES: NCc1ccc2cc(C(=O)O)n(Cc3cc(NC(=O)N4CC5CC(C5)C4)cc4ccccc34)c2n1] | 6-(aminomethyl)-1-{[3- ({3-azabicyclo[3.1.1]- heptane-3-carbonyl}- amino)naphthalen-1-yl]- methyl}-1H-pyrrolo[2,3- b]pyridine-2-carboxylic acid | 470.0 | 95 | C |
| 309 | 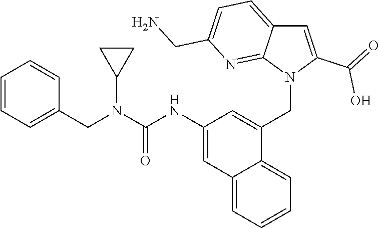 [SMILES: NCc1ccc2cc(C(=O)O)n(Cc3cc(NC(=O)N(Cc4ccccc4)C4CC4)cc4ccccc34)c2n1] | 6-(aminomethyl)-1-[(3- {[benzyl(cyclopropyl)- carbamoyl]amino}naph- thalen-1-yl)methyl]-1H- pyrrolo[2,3-b]pyridine- 2-carboxylic acid | 520.0 | 100 | |
| 310 | 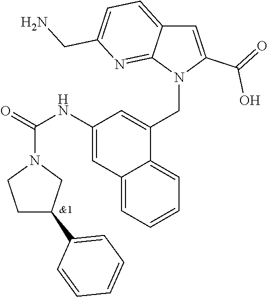 [SMILES: NCc1ccc2cc(C(=O)O)n(Cc3cc(NC(=O)N4CC[C@H](c5ccccc5)C4)cc4ccccc34)c2n1] | rac-6-(aminomethyl)-1- [(3-{[(3R)-3-phenylpyr- rolidine-1-carbonyl]- amino}naphthalen-1-yl)- methyl]-1H-pyrrolo[2,3- b]pyridine-2-carboxylic acid | 520.0 | 100 | |
| 311 | 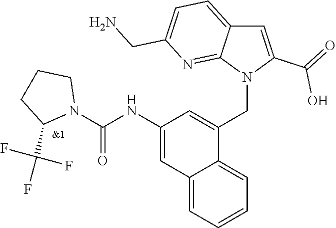 [SMILES: NCc1ccc2cc(C(=O)O)n(Cc3cc(NC(=O)N4CCC[C@H]4C(F)(F)F)cc4ccccc34)c2n1] | rac-6-(aminomethyl)-1- [(3-{[(2R)-2-(trifluoro- methyl)pyrrolidine-1- carbonyl]amino}naph- thalen-1-yl)methyl]-1H- pyrrolo[2,3-b]pyridine- 2-carboxylic acid | 512.0 | 100 | |
| 312 | 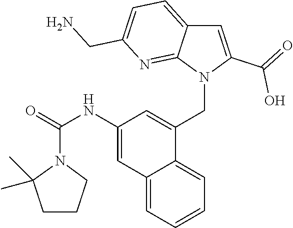 [SMILES: CC1(C)CCCN1C(=O)Nc1cc(Cn2c(C(=O)O)cc3ccc(CN)nc32)c2ccccc2c1] | 6-(aminomethyl)-1-({3- [(2,2-dimethylpyrrol- idine-1-carbonyl)amino]- naphthalen-1-yl}meth- yl)-1H-pyrrolo[2,3-b]- pyridine-2-carboxylic acid | 472.0 | 100 | |
| 313 | 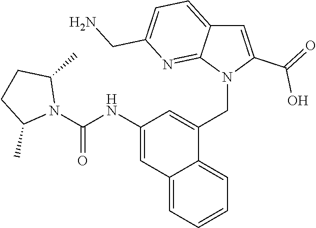 [SMILES: C[C@@H]1CC[C@H](C)N1C(=O)Nc1cc(Cn2c(C(=O)O)cc3ccc(CN)nc32)c2ccccc2c1] | 6-(aminomethyl)-1-[(3- {[(2R,5S)-2,5-dimeth- ylpyrrolidine-1-carbon- yl]amino}naphthalen-1- yl)methyl]-1H-pyrrolo- [2,3-b]pyridine-2-car- boxylic acid | 472.0 | 100 | |
| 314 | 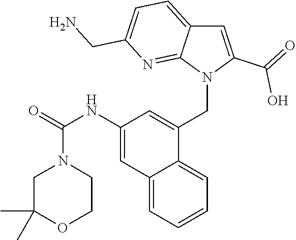 [SMILES: CC1(C)CN(C(=O)Nc2cc(Cn3c(C(=O)O)cc4ccc(CN)nc43)c3ccccc3c2)CCO1] | 6-(aminomethyl)-1-({3- [(2,2-dimethylmorph- oline-4-carbonyl)amino]- naphthalen-1-yl}meth- yl)-1H-pyrrolo[2,3-b]- pyridine-2-carboxylic acid | 488.0 | 100 | |
| 315 | 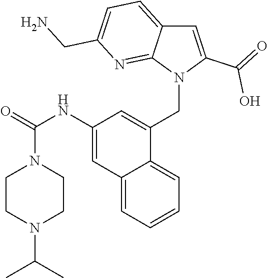 [SMILES: CC(C)N1CCN(C(=O)Nc2cc(Cn3c(C(=O)O)cc4ccc(CN)nc43)c3ccccc3c2)CC1] | 6-(aminomethyl)-1-[(3- {[4-(propan-2-yl)piper- azine-1-carbonyl]amino}- naphthalen-1-yl)methyl]- 1H-pyrrolo[2,3-blpyr- idine-2-carboxylic acid | 501.0 | 99 | |
| 316 | 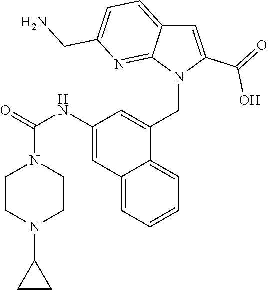 [SMILES: NCc1ccc2cc(C(=O)O)n(Cc3cc(NC(=O)N4CCN(C5CC5)CC4)cc4ccccc34)c2n1] | 6-(aminomethyl)-1-({3- [(4-cyclopropylpiper- azine-1-carbonyl)amino]- naphthalen-1-yl}methyl)- 1H-pyrrolo[2,3-b]pyr- idine-2-carboxylic acid | 499.0 | 99 | |
| 317 | 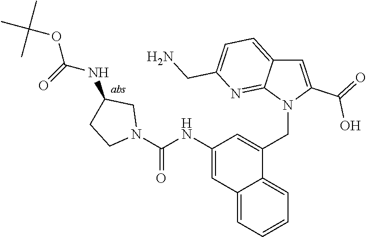 [SMILES: CC(C)(C)OC(=O)N[C@@H]1CCN(C(=O)Nc2cc(Cn3c(C(=O)O)cc4ccc(CN)nc43)c3ccccc3c2)C1] | 6-(aminomethyl)-1-[(3- {[(3R)-3-{[(tert-butoxy)- carbonyl]amino}pyrrol- idine-1-carbonyl]amino}- naphthalen-1-yl)methyl]- 1H-pyrrolo[2,3-b]pyr- idine-2-carboxylic acid | 559.0 | 99 | |
| 318 | 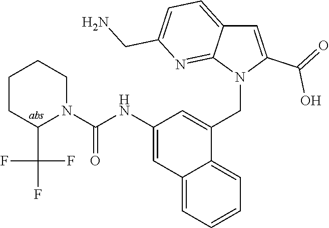 | 6-(aminomethyl)-1-[(3- {[(2R)-2-(trifluorometh- yl)piperidine-1-carbon- yl]amino}naphthalen-1- yl)methyl]-1H-pyrrolo- [2,3-b]pyridine-2-car- boxylic acid | 526.0 | 99 | |
| 319 | 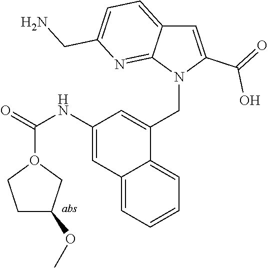 | 6-(aminomethyl)-1-[(3- {[(3S)-3-methoxypyr- rolidine-1-carbonyl]- amino}naphthalen-1-yl)- methyl]-1H-pyrrolo[2,3- b]pyridine-2-carboxylic acid | 474.0 | 99 | |
| 320 | 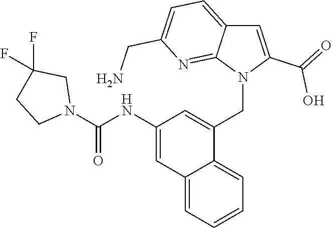 [SMILES: NCc1ccc2cc(C(=O)O)n(Cc3cc(NC(=O)N4CCC(F)(F)C4)cc4ccccc34)c2n1] | 6-(aminomethyl)-1-({3- [(3,3-difluoropyrrol- idine-1-carbonyl)amino]- naphthalen-1-yl}methyl)- 1H-pyrrolo[2,3-b]pyr- idine-2-carboxylic acid | 480.0 | 99 | |
| 321 | 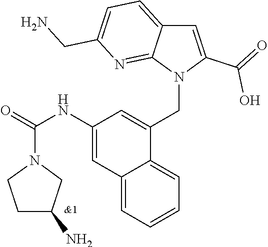 [SMILES: NCc1ccc2cc(C(=O)O)n(Cc3cc(NC(=O)N4CC[C@H](N)C4)cc4ccccc34)c2n1] | rac-6-(aminomethyl)-1- [(3-([(3R)-3-aminopyr- rolidine-1-carbonyl]- amino}naphthalen-1-yl)- methyl]-1H-pyrrolo[2,3- b]pyridine-2-carboxylic acid | 459.0 | 98 | |
| 322 | 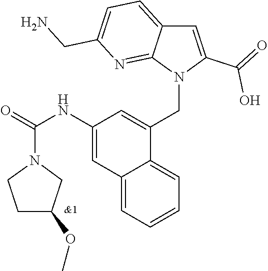 [SMILES: CO[C@H]1CCN(C(=O)Nc2cc(Cn3c(C(=O)O)cc4ccc(CN)nc43)c3ccccc3c2)C1] | rac-6-(aminomethyl)-1- [(3-{[(3R)-3-methoxy- pyrrolidine-1-carbonyl]- amino}naphthalen-1-yl)- methyl]-1H-pyrrolo[2,3- b]pyridine-2-carboxylic acid | 474.0 | 98 | |
| 323 | 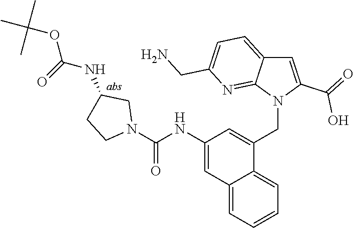 [SMILES: CC(C)(C)OC(=O)N[C@H]1CCN(C(=O)Nc2cc(Cn3c(C(=O)O)cc4ccc(CN)nc43)c3ccccc3c2)C1] | 6-(aminomethyl)-1-[(3- {[(3S)-3-{[(tert-butoxy)- carbonyl]amino}pyrrol- idine-1-carbonyl]amino}- naphthalen-1-yl)methyl]- 1H-pyrrolo[2,3-b]pyr- idine-2-carboxylic acid | 559.0 | 98 | |
| 324 | 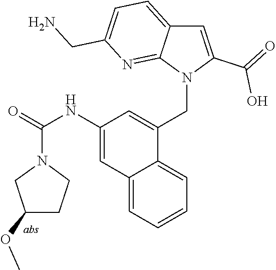 [SMILES: CO[C@@H]1CCN(C(=O)Nc2cc(Cn3c(C(=O)O)cc4ccc(CN)nc43)c3ccccc3c2)C1] | 6-(aminomethyl)-1-[(3- {[(3R)-3-methoxypyr- rolidine-1-carbonyl]- amino}naphthalen-1-yl)- methyl]-1H-pyrrolo[2,3- b]pyridine-2-carboxylic acid | 474.0 | 98 | |
| 325 | 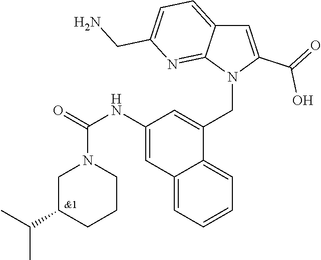 [SMILES: CC(C)[C@H]1CCCN(C(=O)Nc2cc(Cn3c(C(=O)O)cc4ccc(CN)nc43)c3ccccc3c2)C1] | rac-6-(aminomethyl)-1- [(3-{[(3R)-3-(propan-2- yl)piperidine-1-carbon- yl]amino}naphthalen-1- yl)methyl]-1H-pyrrolo- [2,3-blpyridine-2-car- boxylic acid | 500.0 | 97 | |
| 326 | 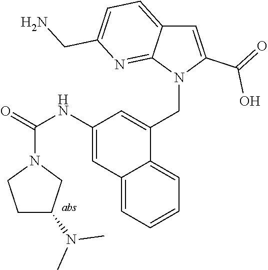 [SMILES: CN(C)[C@@H]1CCN(C(=O)Nc2cc(Cn3c(C(=O)O)cc4ccc(CN)nc43)c3ccccc3c2)C1] | 6-(aminomethyl)-1-[(3- {[(3R)-3-(dimethyl- amino)pyrrolidine-1-car- bonyl]amino}naph- thalen-1-yl)methyl]-1H- pyrrolo[2,3-b]pyridine- 2-carboxylic acid | 487.0 | 97 | |
| 327 | 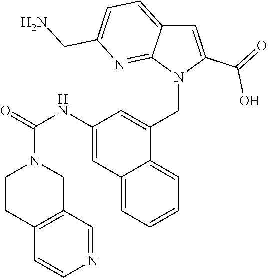 [SMILES: NCc1ccc2cc(C(=O)O)n(Cc3cc(NC(=O)N4CCc5ccncc5C4)cc4ccccc34)c2n1] | 6-(aminomethyl)-1-({3- [(1,2,3,4-tetrahydro-2,7- naphthyridine-2-carbon- yl)amino]naphthalen-1- yl}methyl)-1H-pyrrolo- [2,3-b]pyridine-2-car- boxylic acid | 507.0 | 97 | |
| 328 | 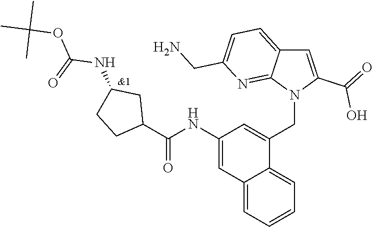 [SMILES: CC(C)(C)OC(=O)N[C@H]1CCC(C(=O)Nc2cc(Cn3c(C(=O)O)cc4ccc(CN)nc43)c3ccccc3c2)C1] | rac-6-(aminomethyl)-1- [(3-{[(3R)-3-{[(tert- butoxy)carbonyl]amino}- pyrrolidine-1-carbonyl]- amino}naphthalen-1-yl)- methyl]-1H-pyrrolo[2,3- b]pyridine-2-carboxylic acid | 559.0 | 95 | |
| 329 | 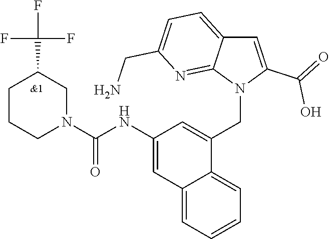 [SMILES: NCc1ccc2cc(C(=O)O)n(Cc3cc(NC(=O)N4CCC[C@H](C(F)(F)F)C4)cc4ccccc34)c2n1] | rac-6-(aminomethyl)-1- [(3-{[(3R)-3-(trifluoro- methyl)piperidine-1-car- bonyl]amino}naph- thalen-1-yl)methyl]-1H- pyrrolo[2,3-b]pyridine- 2-carboxylic acid | 526.0 | 95 | |
| 330 | 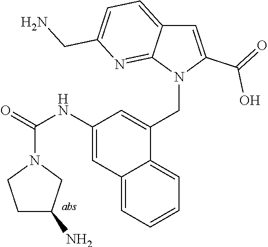 [SMILES: NCc1ccc2cc(C(=O)O)n(Cc3cc(NC(=O)N4CC[C@H](N)C4)cc4ccccc34)c2n1] | 6-(aminomethyl)-1-[(3- {[(3S)-3-aminopyrrol- idine-1-carbonyl]amino}- naphthalen-1-yl)methyl]- 1H-pyrrolo[2,3-b]pyr- idine-2-carboxylic acid | 459.0 | 93 | |
| 331 | 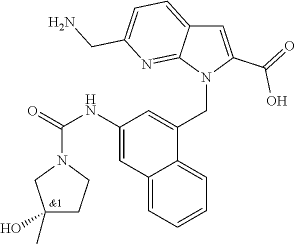 [SMILES: C[C@]1(O)CCN(C(=O)Nc2cc(Cn3c(C(=O)O)cc4ccc(CN)nc43)c3ccccc3c2)C1] | rac-6-(aminomethyl)-1- [(3-{[(3R)-3-hydroxy-3- methylpyrrolidine-1-car- bonyl]amino}naphthalen- 1-yl)methyl]-1H-pyr- rolo[2,3-b]pyridine-2- carboxylic acid | 474.0 | 92 | |
| 332 | 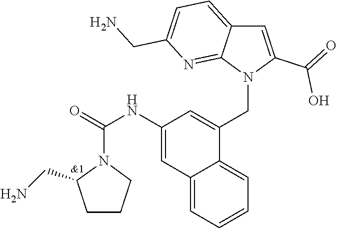 [SMILES: NCc1ccc2cc(C(=O)O)n(Cc3cc(NC(=O)N4CCC[C@@H]4CN)cc4ccccc34)c2n1] | rac-6-(aminomethyl)-1- [(3-{[(2R)-2-(amino- methyl)pyrrolidine-1- carbonyl]amino}naph- naphthalen-1-yl)meth- yl]-1H-pyrrolo[2,3-b]- pyridine-2-carboxylic acid | 473.0 | 91 | |
| 333 | 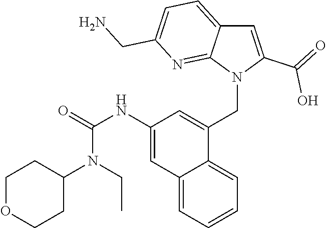 [SMILES: CCN(C(=O)Nc1cc(Cn2c(C(=O)O)cc3ccc(CN)nc32)c2ccccc2c1)C1CCOCC1] | 6-(aminomethyl)-1-[(3- {[ethyl(oxan-4-yl)car- bamoyl]amino}naph- thalen-1-yl)methyl]-1H- pyrrolo[2,3-b]pyridine- 2-carboxylic acid | 502.0 | 88 | |
| 334 | 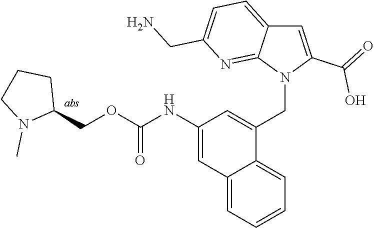 [SMILES: CN1CCC[C@H]1COC(=O)Nc1cc(Cn2c(C(=O)O)cc3ccc(CN)nc32)c2ccccc2c1] | 6-(aminomethyl)-1-({3- [({[(2S)-1-methylpyr- rolidin-2-yl]methoxy}- carbonyl)amino]naph- thalen-1-yl}methyl)- 1H-pyrrolo[2,3-b]pyr- idine-2-carboxylic acid | 488.3 | 99 | B |
| 335 | 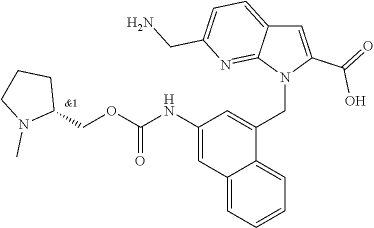 [SMILES: CN1CCC[C@@H]1COC(=O)Nc1cc(Cn2c(C(=O)O)cc3ccc(CN)nc32)c2ccccc2c1] | 6-(aminomethyl)-1-{[3- ({[(1-methylpyrrolidin- 2-yl)methoxy]carbon- yl}amino)naphthalen-1- yl]methyl}-1H-pyrrolo- [2,3-b]pyridine-2-car- boxylic acid | 488.2 | 99 | B |
| 336 | 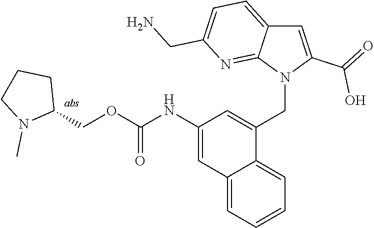 [SMILES: CN1CCC[C@@H]1COC(=O)Nc1cc(Cn2c(C(=O)O)cc3ccc(CN)nc32)c2ccccc2c1] | 6-(aminomethyl)-1-({3- [({[(2R)-1-methylpyr- rolidin-2-yl]methoxy}- carbonyl)amino]naph- thalen-1-yl}methyl)- 1H-pyrrolo[2,3-b]pyr- idine-2-carboxylic acid | 488.2 | 98 | B |
| 337 | 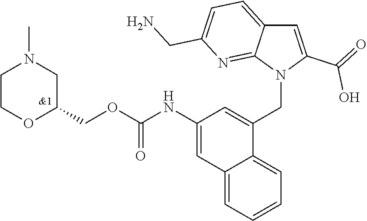 [SMILES: CN1CCO[C@@H](COC(=O)Nc2cc(Cn3c(C(=O)O)cc4ccc(CN)nc43)c3ccccc3c2)C1] | 6-(aminomethyl)-1-{[3- ({[(4-methylmorpholin- 2-yl)methoxy]carbon- yl}amino)naphthalen-1- yl]methyl}-1H-pyrrolo- [2,3-b]pyridine-2-car- boxylic acid | 504.4 | 99 | B |
| 338 | 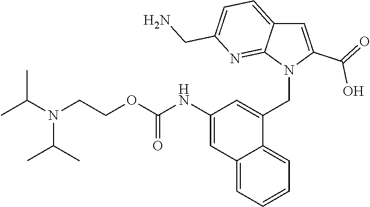 [SMILES: CC(C)N(CCOC(=O)Nc1cc(Cn2c(C(=O)O)cc3ccc(CN)nc32)c2ccccc2c1)C(C)C] | 6-(aminomethyl)-1-({3- [({2-[bis(propan-2-yl)- amino]ethoxy}carbonyl)- amino]naphthalen-1-yl}- methyl)-1H-pyrrolo[2,3- b]pyridine-2-carboxylic acid | 518.0 | 95 | C |
| 339 | 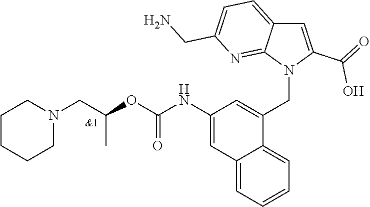 [SMILES: C[C@@H](CN1CCCCC1)OC(=O)Nc1cc(Cn2c(C(=O)O)cc3ccc(CN)nc32)c2ccccc2c1] | rac-6-(aminomethyl)-1- ({3-[({[(2R)-1-(piperidin- 1-yl)propan-2-yl]oxy}- carbonyl)amino]naph- thalen-1-yl}methyl)-1H- pyrrolo[2,3-b]pyridine- 2-carboxylic acid | 516.0 | 97 | C |
| 340 | 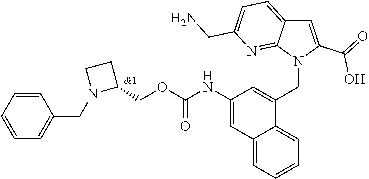 [SMILES: NCc1ccc2cc(C(=O)O)n(Cc3cc(NC(=O)OC[C@H]4CCN4Cc4ccccc4)cc4ccccc34)c2n1] | rac-6-(aminomethyl)-1- ({3-[({[(2R)-1-benzyl- azetidin-2-yl]methoxy}- carbonyl)amino]naph- thalen-1-yl}methyl)-1H- pyrrolo[2,3-b]pyridine- 2-carboxylic acid | 550.0 | 95 | C |
| 341 | 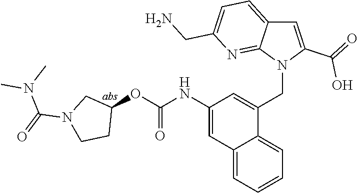 [SMILES: CN(C)C(=O)N1CC[C@H](OC(=O)Nc2cc(Cn3c(C(=O)O)cc4ccc(CN)nc43)c3ccccc3c2)C1] | 6-(aminomethyl)-1-({3- [{[(3S)-1-(dimethylcar- bamoyl)pyrrolidin-3-yl]- oxy}carbonyl)amino]- naphthalen-1-yl}meth- yl)-1H-pyrrolo[2,3-b]- pyridine-2-carboxylic acid | 531.0 | 93 | C |
| 342 | 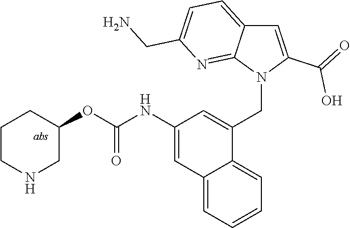 [SMILES: NCc1ccc2cc(C(=O)O)n(Cc3cc(NC(=O)O[C@@H]4CCCNC4)cc4ccccc34)c2n1] | 6-(aminomethyl)-1-{[3- ({[(3R)-piperidin-3-yl- oxy]carbonyl}amino)- naphthalen-1-yl]methyl}- 1H-pyrrolo[2,3-b]pyr- idine-2-carboxylic acid | 474.0 | 85 | |
| 343 | 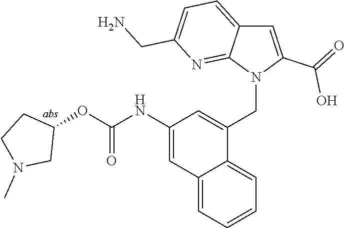 [SMILES: CN1CC[C@H](OC(=O)Nc2cc(Cn3c(C(=O)O)cc4ccc(CN)nc43)c3ccccc3c2)C1] | 6-(aminomethyl)-1-({3- [({[(3S)-1-methylpyr- rolidin-3-yl]oxy}carbon- yl)amino]naphthalen-1- yl}methyl)-1H-pyrrolo- [2,3-b]pyridine-2-car- boxylic acid | 474.0 | 85 | |
| 344 | 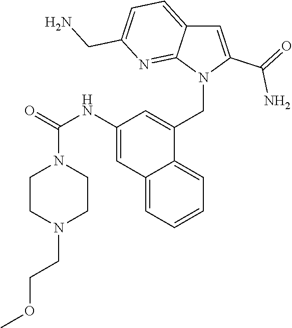 [SMILES: COCCN1CCN(C(=O)Nc2cc(Cn3c(C(N)=O)cc4ccc(CN)nc43)c3ccccc3c2)CC1] | N-(4-{[6-(aminomethyl)- 2-carbamoyl-1H-pyr- rolo[2,3-b]pyridin-1-yl]- methyl}naphthalen-2- yl)-4-(2-methoxyethyl)- piperazine-1-carboxamide | 516.3 | 100 | B |
| 345 | 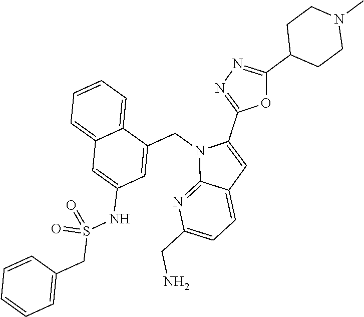 [SMILES: CN1CCC(c2nnc(-c3cc4ccc(CN)nc4n3Cc3cc(NS(=O)(=O)Cc4ccccc4)cc4ccccc34)o2)CC1] | N-(4-{[6-(aminomethyl)- 2-[5-(1-methylpiperidin- 4-yl)-1,3,4-oxadiazol-2- yl]-1H-pyrrolo[2,3-b]- pyridin-1-yl]methyl}- naphthalen-2-yl)-1-phen- ylmethanesulfonamide | 622.4 | 98 | A |
| 346 | 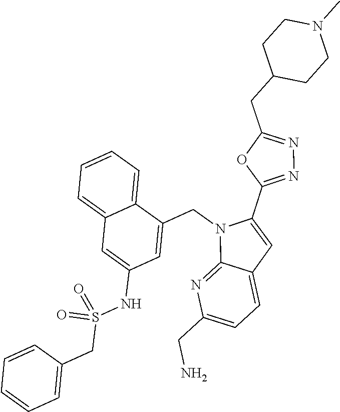 [SMILES: CN1CCC(Cc2nnc(-c3cc4ccc(CN)nc4n3Cc3cc(NS(=O)(=O)Cc4ccccc4)cc4ccccc34)o2)CC1] | N-(4-{[6-(aminomethyl)- 2-{5-[(1-methylpiperidin- 4-yl)methyl]-1,3,4-oxa- diazol-2-yl}-1H-pyrrolo- [2,3-b]pyridin-1-yl]meth- yl}naphthalen-2-yl)-1- phenylmethanesulfon- amide | 636.4 | 99 | B |
| 347 | 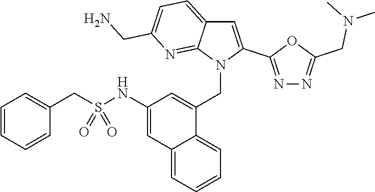 [SMILES: CN(C)Cc1nnc(-c2cc3ccc(CN)nc3n2Cc2cc(NS(=O)(=O)Cc3ccccc3)cc3ccccc23)o1] | N-(4-{[6-(aminomethyl)- 2-{5-[(dimethylamino)- methyl]-1,3,4-oxadiazol- 2-yl}-1H-pyrrolo[2,3- b]pyridin-1-yl]methyl}- naphthalen-2-yl)-1-phen- ylmethanesulfonamide | 582.4 | 96 | B |
| 348 | 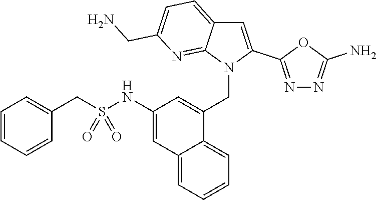 [SMILES: NCc1ccc2cc(-c3nnc(N)o3)n(Cc3cc(NS(=O)(=O)Cc4ccccc4)cc4ccccc34)c2n1] | N-(4-{[2-(5-amino-1,3,4- oxadiazol-2-yl)-6-(amino- methyl)-1H-pyrrolo[2,3- b]pyridin-1-yl]methyl}- naphthalen-2-yl)-1-phen- ylmethanesulfonamide | 540.3 | 97 | A |
| 349 | 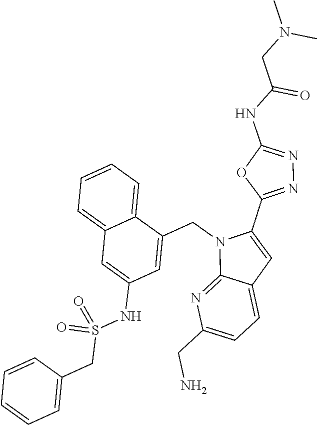 [SMILES: CN(C)CC(=O)Nc1nnc(-c2cc3ccc(CN)nc3n2Cc2cc(NS(=O)(=O)Cc3ccccc3)cc3ccccc23)o1] | N-{5-[6-(aminomethyl)- 1-{[3-(phenylmethane- sulfonamido)naphthalen- 1-yl]methyl}-1H-pyr- rolo[2,3-b]pyridin-2-yl]- 1,3,4-oxadiazol-2-yl}-2- (dimethylamino)acet- amide | 625.4 | 97 | A |
| 350 | 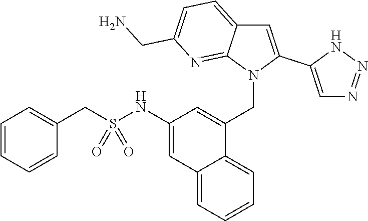 [SMILES: NCc1ccc2cc(-c3cnn[nH]3)n(Cc3cc(NS(=O)(=O)Cc4ccccc4)cc4ccccc34)c2n1] | N-(4-{[6-(aminomethyl)- 2-(1H-1,2,3-triazol-5-yl)- 1H-pyrrolo[2,3-b]pyridin- 1-yl]methyl}naphthalen- 2-yl)-1-phenylmethane- sulfonamide | 524.3 | 84 | B |
| 351 | 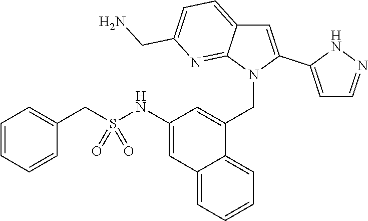 [SMILES: NCc1ccc2cc(-c3ccn[nH]3)n(Cc3cc(NS(=O)(=O)Cc4ccccc4)cc4ccccc34)c2n1] | N-(4-{[6-(aminomethyl)- 2-(1H-pyrazol-5-yl)-1H- pyrrolo[2,3-b]pyridin-1- yl]methyl}naphthalen-2- yl)-1-phenylmethane- sulfonamide | 523.3 | 97 | A |
| 352 | 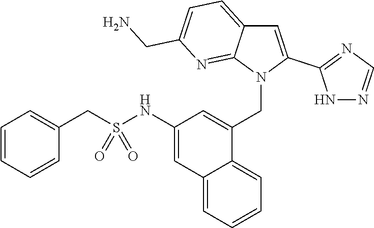 [SMILES: NCc1ccc2cc(-c3ncn[nH]3)n(Cc3cc(NS(=O)(=O)Cc4ccccc4)cc4ccccc34)c2n1] | N-(4-{[6-(aminomethyl)- 2-(1H-1,2,4-triazol-5-yl)- 1H-pyrrolo[2,3-b]pyridin- 1-ylmethyl}naphthalen- 2-yl)-1-phenylmethane- sulfonamide | 524.3 | 97 | A |
| 353 | 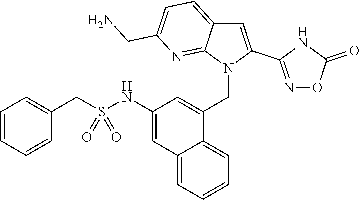 [SMILES: NCc1ccc2cc(-c3noc(=O)[nH]3)n(Cc3cc(NS(=O)(=O)Cc4ccccc4)cc4ccccc34)c2n1] | N-(4-{[6-(aminomethyl)- 2-(5-oxo-4,5-dihydro- 1,2,4-oxadiazol-3-yl)- 1H-pyrrolo[2,3-b]pyridin- 1-yl]methyl}naphthalen- 2-yl)-1-phenylmethane- sulfonamide | 541.2 | 95 | B |
| 354 | 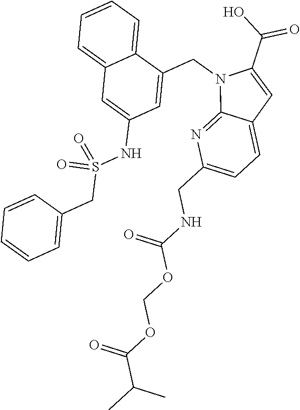 [SMILES: CC(C)C(=O)OCOC(=O)NCc1ccc2cc(C(=O)O)n(Cc3cc(NS(=O)(=O)Cc4ccccc4)cc4ccccc34)c2n1] | 6-{[({[(2-methylpropan- oyl)oxy]methoxy}car- bonyl)amino]methyl}-1- 1{[3-(phenylmethane- sulfonamido)naphthalen- 1-yl]methyl}-1H-pyr- rolo-[2,3-b]pyridine-2- carboxylic acid | 645.3 | 99 | |
| 355 | 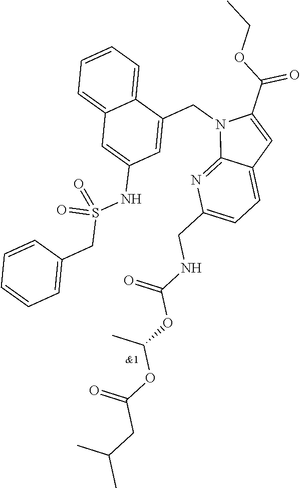 [SMILES: CCOC(=O)c1cc2ccc(CNC(=O)O[C@@H](C)OC(=O)CC(C)C)nc2n1Cc1cc(NS(=O)(=O)Cc2ccccc2)cc2ccccc12] | ethyl 6-{[({1-[(3-methyl- butanoyl)oxy]ethoxy}- carbonyl)aminomethyl}- 1-{[3-(phenylmethane- sulfonamido)naphthalen- 1-yl]methyl}-1H-pyrrolo- [2,3-b]pyridine-2-car- boxylate | 701.5 | 90 | |
| 356 | 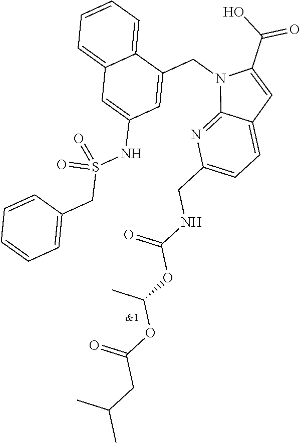 [SMILES: CC(C)CC(=O)O[C@H](C)OC(=O)NCc1ccc2cc(C(=O)O)n(Cc3cc(NS(=O)(=O)Cc4ccccc4)cc4ccccc34)c2n1] | 6-{[({1-[(2-methyl- propanoyl)oxy]ethoxy}- carbonyl)amino]methyl}- 1-{[3-(phenylmethane- sulfonamido)naphthalen- 1-yl]methyl}-1H-pyr- rolo[2,3-b]pyridine-2- carboxylic acid | 659.4 | 98 | |
| 357 | 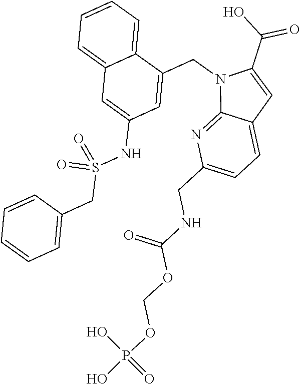 [SMILES: O=C(NCc1ccc2cc(C(=O)O)n(Cc3cc(NS(=O)(=O)Cc4ccccc4)cc4ccccc34)c2n1)OCOP(=O)(O)O] | 1-{[3-(phenylmethane- sulfonamido)naphthalen- 1-yl]methyl}-6-[({[(phos- phonooxy)methoxy]car- bonyl}amino)methyl]- 1H-pyrrolo[2,3-b]pyr- idine-2-carboxylic acid | 655.2 | 80 | |
| 358 | 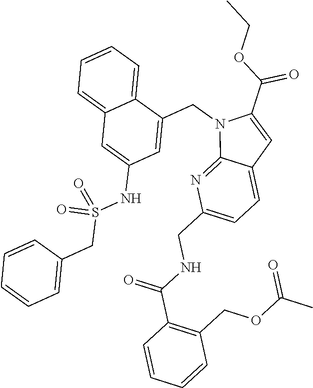 [SMILES: CCOC(=O)c1cc2ccc(CNC(=O)c3ccccc3COC(C)=O)nc2n1Cc1cc(NS(=O)(=O)Cc2ccccc2)cc2ccccc12] | ethyl 6-[({2-[(acetyl- oxy)methyl]phenyl}- formamido)methyl]-1- {[3-(phenylmethane- sulfonamido)naphthalen- 1-yl]methyl}-1H-pyr- rolo[2,3-b]pyridine-2- carboxylate | 705.3 | 98 | C |
| 359 | 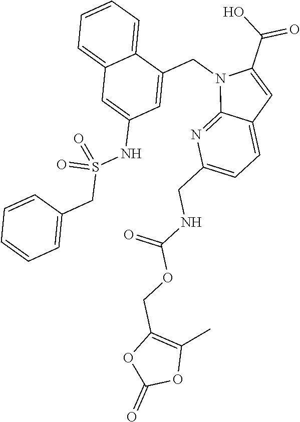 [SMILES: Cc1oc(=O)oc1COC(=O)NCc1ccc2cc(C(=O)O)n(Cc3cc(NS(=O)(=O)Cc4ccccc4)cc4ccccc34)c2n1] | 6-{({[(5-methyl-2-oxo- 2H-1,3-dioxol-4-yl)meth- oxy]carbonyl}amino)- methyl]-1-1[3-(phenyl- methanesulfonamido)- naphthalen-1-yl]methyl}- 1H-pyrrolo[2,3-b]pyr- idine-2-carboxylic acid | 657.3 | 99 | B |
| 360 | 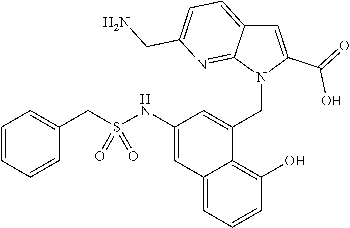 [SMILES: NCc1ccc2cc(C(=O)O)n(Cc3cc(NS(=O)(=O)Cc4ccccc4)cc4cccc(O)c34)c2n1] | 6-(aminomethyl)-1-{[8- hydroxy-3-(phenyl- methanesulfonamido)- naphthalen-1-yl]meth- yl}-1H-pyrrolo[2,3-b]- pyridine-2-carboxylic acid | 517.3 | 95 | B |
| 361 | 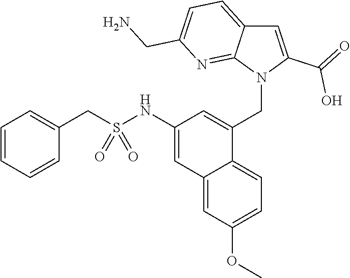 [SMILES: COc1ccc2c(Cn3c(C(=O)O)cc4ccc(CN)nc43)cc(NS(=O)(=O)Cc3ccccc3)cc2c1] | 6-(aminomethyl)-1-{[6- methoxy-3-(phenyl- methanesulfonamido)- naphthalen-1-yl]methyl}- 1H-pyrrolo[2,3-b]pyr- idine-2-carboxylic acid | 531.3 | 96 | B |
| 362 | 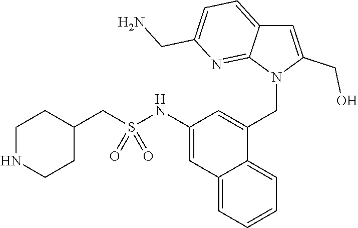 [SMILES: NCc1ccc2cc(CO)n(Cc3cc(NS(=O)(=O)CC4CCNCC4)cc4ccccc34)c2n1] | N-(4-{[6-(aminomethyl)- 2-(hydroxymethyl)-1H- pyrrolo[2,3-b]pyridin-1- yl]methyl}naphthalen-2- yl)-1-(piperidin-4-yl)- methanesulfonamide | 494.3 | 97 | B |
| 363 | 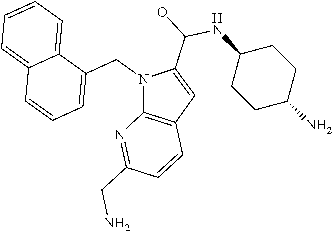 [SMILES: NCc1ccc2cc(C(O)N[C@H]3CC[C@H](N)CC3)n(Cc3cccc4ccccc34)c2n1] | 6-(aminomethyl)-1- [(naphthalen-1-yl)meth- yl]-N-[(1r,4r)-4-amino- cyclohexyl]-1H-pyrrolo- [2,3-b]pyridine-2-car- boxamide | 428.0 | 95 | C |
| 364 | 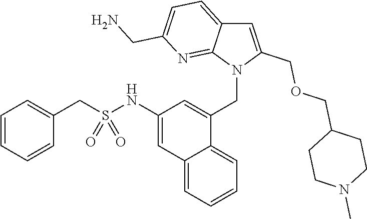 [SMILES: CN1CCC(COCc2cc3ccc(CN)nc3n2Cc2cc(NS(=O)(=O)Cc3ccccc3)cc3ccccc23)CC1] | N-(4-{[6-(aminomethyl)- 2-{[(1-methylpiperidin- 4-yl)methoxy]methyl}- 1H-pyrrolo[2,3-b]pyri- din-1-yl]methyl}naph- thalen-2-yl)-1-phenyl- methanesulfonamide | 598.4 | 99 | C |
| 365 | 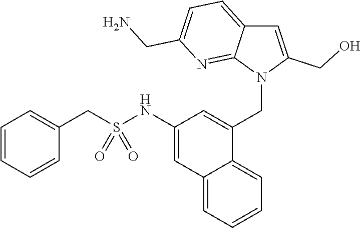 [SMILES: NCc1ccc2cc(CO)n(Cc3cc(NS(=O)(=O)Cc4ccccc4)cc4ccccc34)c2n1] | N-(4-{[6-(aminomethyl)- 2-(hydroxymethyl)-1H- pyrrolo[2,3-b]pyridin-1- yl]methyl}naphthalen-2- yl)-1-phenylmethane- sulfonamide | 487.2 | 96 | C |
| 366 | 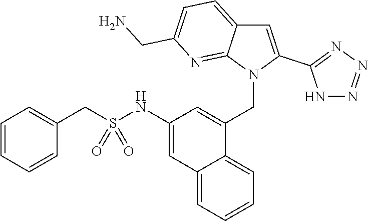 [SMILES: NCc1ccc2cc(-c3nnn[nH]3)n(Cc3cc(NS(=O)(=O)Cc4ccccc4)cc4ccccc34)c2n1] | N-(4-{[6-(aminomethyl)- 2-(1H-1,2,3,4-tetrazo1- 5-yl)-1H-pyrrolo[2,3-b]- pyridin-1-yl]methyl}- naphthalen-2-yl)-1-phen- ylmethanesulfonamide | 525.3 | 99 | A |
| 367 | 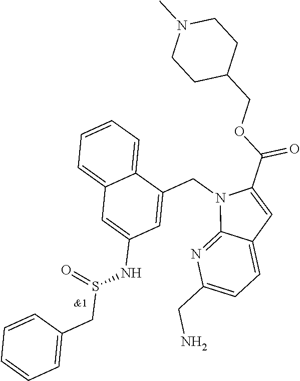 [SMILES: CN1CCC(COC(=O)c2cc3ccc(CN)nc3n2Cc2cc(N[S@@](=O)Cc3ccccc3)cc3ccccc23)CC1] | (1-methylpiperidin-4-yl)- methyl 6-(aminometh- yl)-1-{3-(phenylmeth- anesulfinylamino)naph- thalen-1-yl]methyl}-1H- pyrrolo[2,3-b]pyridine- 2-carboxylate | 596.4 | 80 | C |
| 368 | 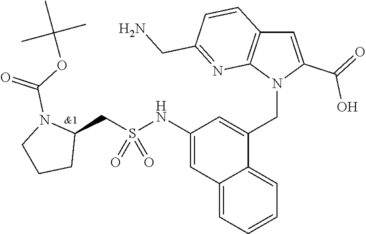 [SMILES: CC(C)(C)OC(=O)N1CCC[C@@H]1CS(=O)(=O)Nc1cc(Cn2c(C(=O)O)cc3ccc(CN)nc32)c2ccccc2c1] | 6-(aminomethyl)-1-{[3- ({1-[(tert-butoxy)car- bonyl]pyrrolidin-2-yl} methanesulfonamido)- naphthalen-1-yl]methyl}- 1H-indole-2-carboxylic acid | 593.1 | 94 | |
| 369 | 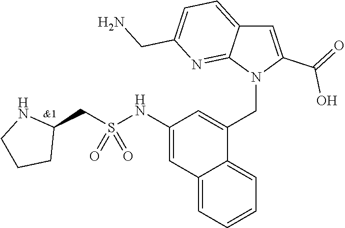 [SMILES: NCc1ccc2cc(C(=O)O)n(Cc3cc(NS(=O)(=O)C[C@H]4CCCN4)cc4ccccc34)c2n1] | 6-(aminomethyl)-1-({3- [(pyrrolidin-2-yl)meth- anesulfonamido]naph- thalen-1-yl}methyl)-1H- indole-2-carboxylic acid | 493.1 | 100 | B |
| 370 | 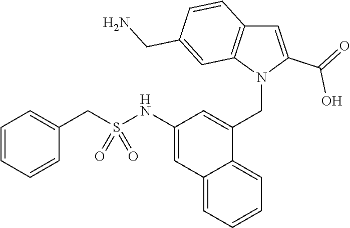 [SMILES: NCc1ccc2cc(C(=O)O)n(Cc3cc(NS(=O)(=O)Cc4ccccc4)cc4ccccc34)c2c1] | 6-(aminomethyl)-1-{[3- (phenylmethanesulfon- amido)naphthalen-1-yl]- methyl}-1H-indole-2- carboxylic acid | 500.0 | 100 | A |
| 371 | 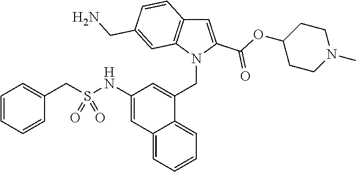 [SMILES: CN1CCC(OC(=O)c2cc3ccc(CN)cc3n2Cc2cc(NS(=O)(=O)Cc3ccccc3)cc3ccccc23)CC1] | 1-methylpiperidin-4-yl 6-(aminomethyl)-1-{[3- (phenylmethanesulfon- amido)naphthalen-1-yl]- methyl}-1H-indole-2- carboxylate | 596.4 | 100 | A |
| 372 | 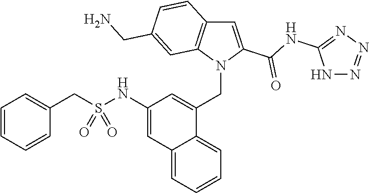 [SMILES: NCc1ccc2cc(C(=O)Nc3nnn[nH]3)n(Cc3cc(NS(=O)(=O)Cc4ccccc4)cc4ccccc34)c2c1] | 6-(aminomethyl)-1-{[3- (phenylmethanesulfon- amido)naphthalen-1-yl]- methyl}-N-(1H-1,2,3,4- tetrazol-5-yl)-1H-indole- 2-carboxamide | 567.1 | 100 | B |
| 373 | 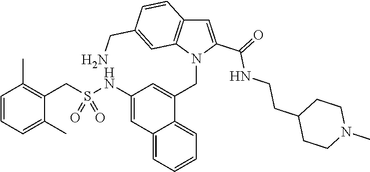 [SMILES: Cc1cccc(C)c1CS(=O)(=O)Nc1cc(Cn2c(C(=O)NCCC3CCN(C)CC3)cc3ccc(CN)cc32)c2ccccc2c1] | 6-(aminomethyl)-1-({3- [(2,6-dimethylphenyl)- methanesulfonamido]- naphthalen-1-yl}methyl)- N-[2-(1-methylpiperidin- 4-yl)ethyl]-1H-indole-2- carboxamide | 650.3 | 99 | A |
| 374 | 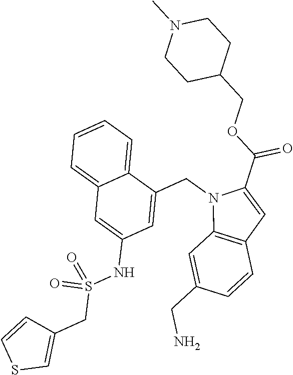 [SMILES: CN1CCC(COC(=O)c2cc3ccc(CN)cc3n2Cc2cc(NS(=O)(=O)Cc3ccsc3)cc3ccccc23)CC1] | (1-methylpiperidin-4-yl)- methyl 6-(aminomethyl)- 1-({3-[(thiophen-3-yl)- methanesulfonamido]- naphthalen-1-yl}methyl)- 1H-indole-2-carboxylate | 617.1 | 97 | A |
| 375 | 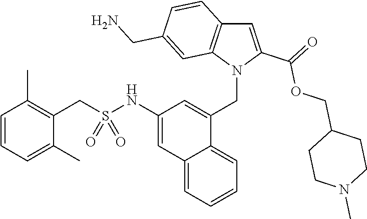 [SMILES: Cc1cccc(C)c1CS(=O)(=O)Nc1cc(Cn2c(C(=O)OCC3CCN(C)CC3)cc3ccc(CN)cc32)c2ccccc2c1] | (1-methylpiperidin-4-yl)- methyl 6-(aminomethyl)- 1-({3-[(2,6-dimethyl- phenyl)methanesulfon- amido)naphthalen-1-yl}- methyl)-1H-indole-2- carboxylate | 639.5 | 97 | A |
| 376 | 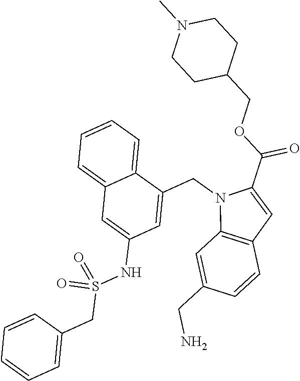 [SMILES: CN1CCC(COC(=O)c2cc3ccc(CN)cc3n2Cc2cc(NS(=O)(=O)Cc3ccccc3)cc3ccccc23)CC1] | (1-methylpiperidin-4-yl)- methyl 6-(aminomethyl)- 1-1{[3-(phenylmethane- sulfonamido)naphthalen- 1-yl]methyl}-1H-indole- 2-carboxylate | 611.2 | 98 | A |
| 377 | 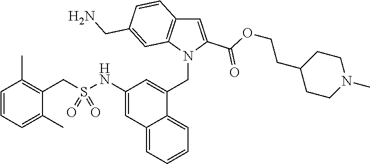 [SMILES: Cc1cccc(C)c1CS(=O)(=O)Nc1cc(Cn2c(C(=O)OCCC3CCN(C)CC3)cc3ccc(CN)cc32)c2ccccc2c1] | 2-(1-methylpiperidin-4- yl)ethyl 6-(aminometh- yl)-1-({3-[(2,6-dimethyl- phenyl)methanesulfon- amido]naphthalen-1-yl}- methyl)-1H-indole-2- carboxylate | 653.5 | 96 | A |
| 378 | 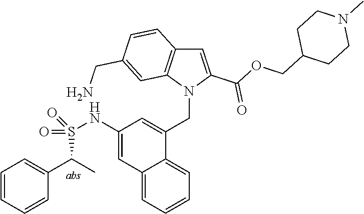 [SMILES: C[C@H](c1ccccc1)S(=O)(=O)Nc1cc(Cn2c(C(=O)OCC3CCN(C)CC3)cc3ccc(CN)cc32)c2ccccc2c1] | (1-methylpiperidin-4-yl)- methyl 6-(aminomethyl)- 1-({3-[(1R)-1-phenyl- ethane-sulfonamido]- naphthalen-1-yl}methyl)- 1H-indole-2-carboxylate | 625.5 | 96 | A |
| 379 | 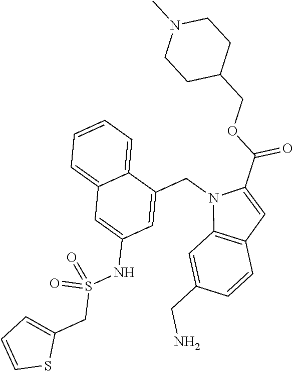 [SMILES: CN1CCC(COC(=O)c2cc3ccc(CN)cc3n2Cc2cc(NS(=O)(=O)Cc3cccs3)cc3ccccc23)CC1] | (1-methylpiperidin-4-yl)- methyl 6-(aminomethyl)- 1-({3-[(thiophen-2-yl)- methanesulfonamido]- naphthalen-1-yl}meth- yl)-1H-indole-2-carbox- ylate | 617.4 | 97 | A |
| 380 | 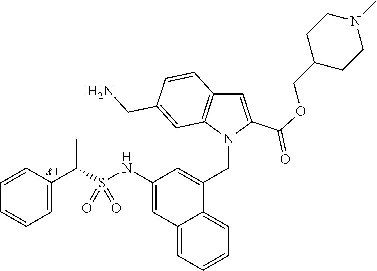 [SMILES: C[C@@H](c1ccccc1)S(=O)(=O)Nc1cc(Cn2c(C(=O)OCC3CCN(C)CC3)cc3ccc(CN)cc32)c2ccccc2c1] | (1-methylpiperidin-4-yl)- methyl 6-(aminomethyl)- 1-{[3-(1-phenylethane- sulfonamido)naphthalen- 1-yl]methyl}-1H-indole- 2-carboxylate | 625.5 | 99 | A |
| 381 | 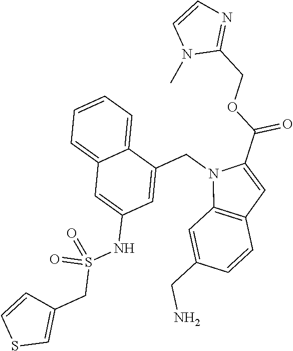 [SMILES: Cn1ccnc1COC(=O)c1cc2ccc(CN)cc2n1Cc1cc(NS(=O)(=O)Cc2ccsc2)cc2ccccc12] | (1-methyl-1H-imidazol- 2-yl)methyl 6-(amino- methyl)-1-({3-[(thio- phen-3-yl)methanesulfon- amido]naphthalen-1-yl}- methyl)-1H-indole-2- carboxylate | 598.1 | 99 | A |
| 382 | 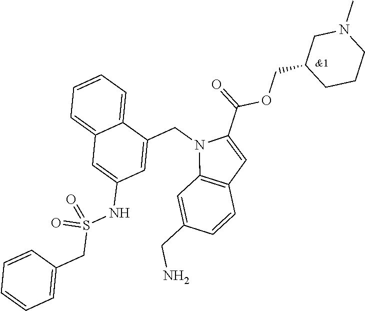 [SMILES: CN1CCC[C@H](COC(=O)c2cc3ccc(CN)cc3n2Cc2cc(NS(=O)(=O)Cc3ccccc3)cc3ccccc23)C1] | (1-methylpiperidin-3-yl)- methyl 6-(aminomethyl)- 1-{[3-(phenylmethane- sulfonamido)naphthalen- 1-yl]methyl}-1H-indole- 2-carboxylate | 611.1 | 100 | A |
| 383 | 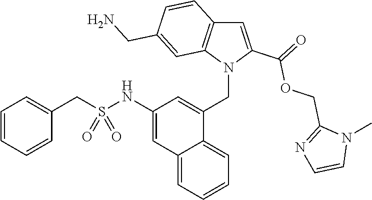 [SMILES: Cn1ccnc1COC(=O)c1cc2ccc(CN)cc2n1Cc1cc(NS(=O)(=O)Cc2ccccc2)cc2ccccc12] | (1-methyl-1H-imidazol- 2-yl)methyl 6-(amino- methyl)-1-{[3-(phenyl- methanesulfonamido)- naphthalen-1-yl]meth- yl}-1H-indole-2-carbox- ylate | 594.0 | 98 | A |
| 384 | 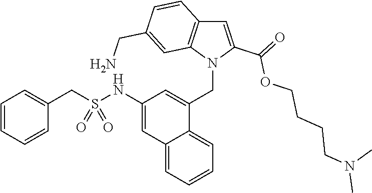 [SMILES: CN(C)CCCCOC(=O)c1cc2ccc(CN)cc2n1Cc1cc(NS(=O)(=O)Cc2ccccc2)cc2ccccc12] | 4-(dimethylamino)butyl 6-(aminomethyl)-1-{[3- (phenylmethanesulfon- amido)naphthalen-1-yl]- methyl}-1H-indole-2- carboxylate | 599.4 | 99 | B |
| 385 | 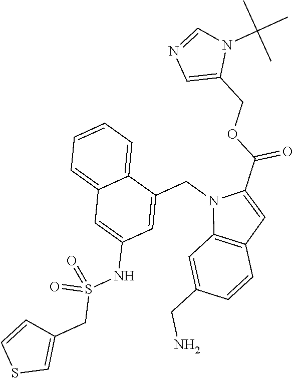 [SMILES: CC(C)(C)n1cncc1COC(=O)c1cc2ccc(CN)cc2n1Cc1cc(NS(=O)(=O)Cc2ccsc2)cc2ccccc12] | (1-tert-butyl-1H-imid- azol-5-yl)methyl 6- (aminomethyl)-1-({3- [(thiophen-3-yl)methane- sulfonamido]naphthalen- 1-yl}methyl)-1H-indole- 2-carboxylate | 642.0 | 97 | B |
| 386 | 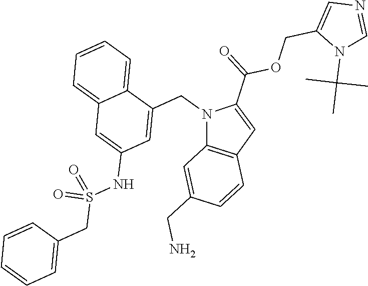 [SMILES: CC(C)(C)n1cncc1COC(=O)c1cc2ccc(CN)cc2n1Cc1cc(NS(=O)(=O)Cc2ccccc2)cc2ccccc12] | (1-tert-butyl-1H-imid- azol-5-yl)methyl 6- (aminomethyl)-1-{[3- (phenylmethanesulfon- amido)naphthalen-1-yl]- methyl}-1H-indole-2- carboxylate | 636.1 | 97 | B |
| 387 | 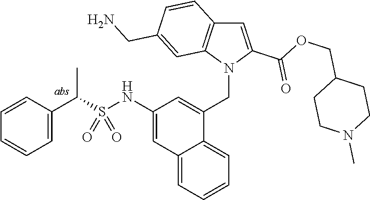 [SMILES: C[C@@H](c1ccccc1)S(=O)(=O)Nc1cc(Cn2c(C(=O)OCC3CCN(C)CC3)cc3ccc(CN)cc32)c2ccccc2c1] | (1-methylpiperidin-4-yl)- methyl 6-(aminomethyl)- 1-({3-[(1S)-1-phenyl- ethanesulfonamido]- naphthalen-1-yl}methyl)- 1H-indole-2-carboxylate | 625.5 | 100 | B |
| 388 | 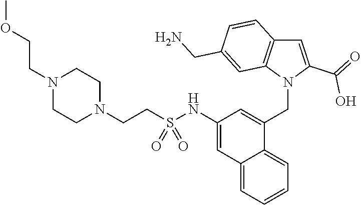 [SMILES: COCCN1CCN(CCS(=O)(=O)Nc2cc(Cn3c(C(=O)O)cc4ccc(CN)cc43)c3ccccc3c2)CC1] | 6-(aminomethyl)-1-[(3- {2-[4-(2-methoxyethyl)- piperazin-1-yl]ethanesul- fonamido}naphthalen-1- yl)methyl]-1H-indole-2- carboxylic acid | 580.1 | 98 | B |
| 389 | 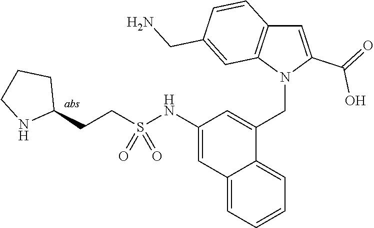 [SMILES: NCc1ccc2cc(C(=O)O)n(Cc3cc(NS(=O)(=O)CC[C@@H]4CCCN4)cc4ccccc34)c2c1] | 6-(aminomethyl)-1-[(3- {2-[(2S)-pyrrolidin-2- yl]ethanesulfonamido}- naphthalen-1-yl)methyl]- 1H-indole-2-carboxylic acid | 507.0 | 96 | B |
| 390 | 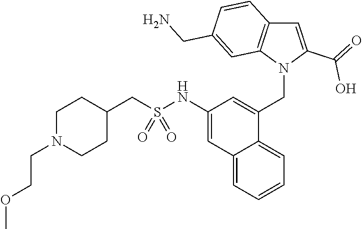 [SMILES: COCCN1CCC(CS(=O)(=O)Nc2cc(Cn3c(C(=O)O)cc4ccc(CN)cc43)c3ccccc3c2)CC1] | 6-(aminomethyl)-1-[(3- {[1-(2-methoxyethyl)- piperidin-4-yl]methane- sulfonamido}naphthalen- 1-yl)methyl]-1H-indole- 2-carboxylic acid | 565.1 | 92 | B |
| 391 | 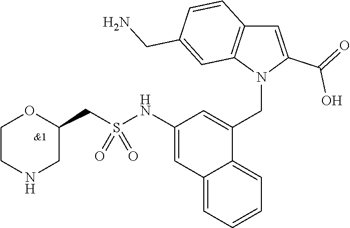 [SMILES: NCc1ccc2cc(C(=O)O)n(Cc3cc(NS(=O)(=O)C[C@H]4CNCCO4)cc4ccccc34)c2c1] | 6-(aminomethyl)-1-({3- [(morpholin-2-l)methane- sulfonamido]naphthalen- 1-yl}methyl)-1H-indole- 2-carboxylic acid | 509.0 | 99 | B |
| 392 | 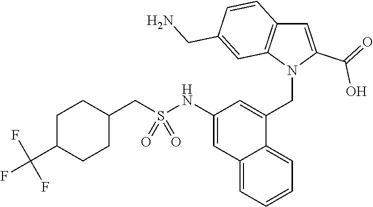 [SMILES: NCc1ccc2cc(C(=O)O)n(Cc3cc(NS(=O)(=O)CC4CCC(C(F)(F)F)CC4)cc4ccccc34)c2c1] | 6-(aminomethyl)-1-1(3- {[4-(trifluoromethyl)- cyclohexyl]methanesul- fonamido}naphthalen-1- yl)methyl]-1H-indole-2- carboxylic acid | 573.9 | 96 | C |
| 393 | 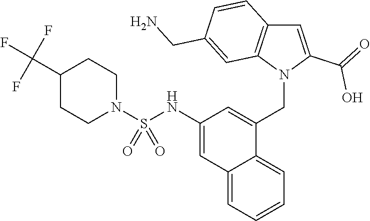 [SMILES: NCc1ccc2cc(C(=O)O)n(Cc3cc(NS(=O)(=O)N4CCC(C(F)(F)F)CC4)cc4ccccc34)c2c1] | 6-(aminomethyl)-1-{[3- ({[4-(trifluoromethyl)- piperidin-1-yl]sulfonyl}- amino)naphthalen-1-yl]- methyl}-1H-indole-2- carboxylic acid | 561.0 | 98 | C |
| 394 | 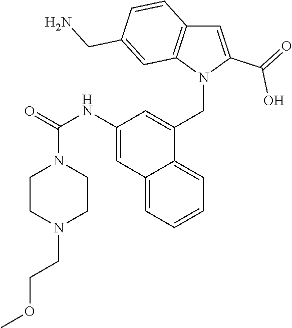 [SMILES: COCCN1CCN(C(=O)Nc2cc(Cn3c(C(=O)O)cc4ccc(CN)cc43)c3ccccc3c2)CC1] | 6-(aminomethyl)-1-[(3- {[4-(2-methoxyethyl)- piperazine-1-carbonyl]- amino}naphthalen-1-yl)- methyl]-1H-indole-2- carboxylic acid | 516.2 | 99 | C |
| 395 | 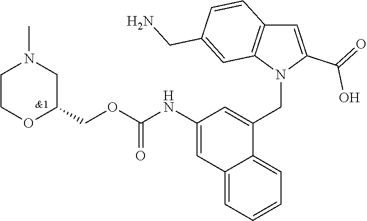 [SMILES: CN1CCO[C@@H](COC(=O)Nc2cc(Cn3c(C(=O)O)cc4ccc(CN)cc43)c3ccccc3c2)C1] | 6-(aminomethyl)-1-{[3- ({[(4-methylmorpholin- 2-yl)methoxy]carbonyl}- amino)naphthalen-1-yl]- methyl}-1H-indole-2- carboxylic acid | 503.2 | 96 | C |
| 396 | 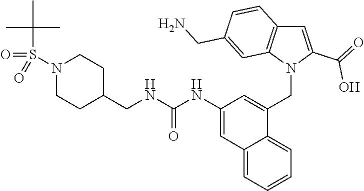 [SMILES: CC(C)(C)S(=O)(=O)N1CCC(CNC(=O)Nc2cc(Cn3c(C(=O)O)cc4ccc(CN)cc43)c3ccccc3c2)CC1] | 6-(aminomethyl)-1-({3- [({[1-(2-methylpropane- 2-sulfonyl)piperidin-4- yl]methyl}carbamoyl)- amino]naphthalen-1-yl}- methyl)-1H-indole-2- carboxylic acid | 606.0 | 99 | B |
| 397 | 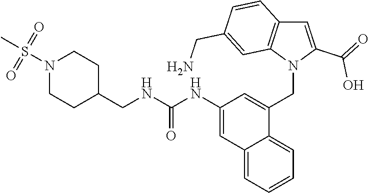 [SMILES: CS(=O)(=O)N1CCC(CNC(=O)Nc2cc(Cn3c(C(=O)O)cc4ccc(CN)cc43)c3ccccc3c2)CC1] | 6-(aminomethyl)-1-{[3- ({[(1-methanesulfonyl- piperidin-4-yl)methyl]- carbamoyl}amino)naph- thalen-1-yl]methyl}-1H- indole-2-carboxylic acid | 564.2 | 95.97 | B |
| 398 | 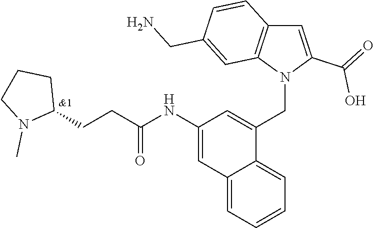 [SMILES: CN1CCC[C@@H]1CCC(=O)Nc1cc(Cn2c(C(=O)O)cc3ccc(CN)cc32)c2ccccc2c1] | 6-(aminomethyl)-1-{[3- ({[(1-methylpyrrolidin- 2-yl)methoxy]carbon- yl}amino]naphthalen-1- yl]methyl}-1H-indole-2- carboxylic acid | 487.5 | 93 | C |
| 399 | 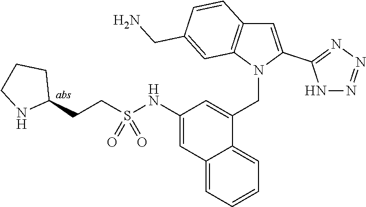 [SMILES: NCc1ccc2cc(-c3nnn[nH]3)n(Cc3cc(NS(=O)(=O)CC[C@@H]4CCCN4)cc4ccccc34)c2c1] | N-(4-{[6-(aminomethyl)- 2-(1H-1,2,3,4-tetrazol-5- yl)-1H-indol-1-yl]meth- yl}naphthalen-2-yl)-2- [(2S)-pyrrolidin-2-yl]- ethane-1-sulfonamide | 531.3 | 93 | B |

For Table B. (i) Kᵢ≤0.1 μM is indicated as A. (ii) 0.1 μM<Kᵢ≤0.5 μM is indicated as B. (iii) 0.5 μM<K≤3 μM is indicated as C; and (iv) Kᵢ>3 μM is indicated as D.

TABLE B

| Cpmd | | Mass by LCMS | Mean Kᵢ |
|---|---|---|---|
| # | Structure | [M + 1]+ | (uM) |
| 2.1 | 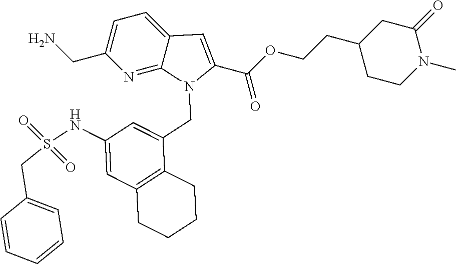 [SMILES: CN1CCC(CCOC(=O)c2cc3ccc(CN)nc3n2Cc2cc(NS(=O)(=O)Cc3ccccc3)cc3c2CCCC3)CC1=O] | 644.2 | B |
| 2.2 | 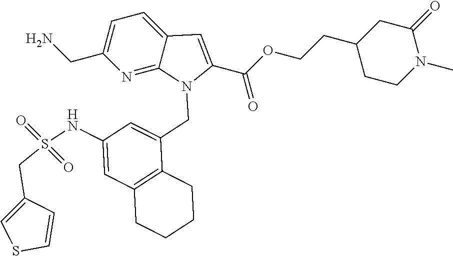 [SMILES: CN1CCC(CCOC(=O)c2cc3ccc(CN)nc3n2Cc2cc(NS(=O)(=O)Cc3ccsc3)cc3c2CCCC3)CC1=O] | 650.15 | B |
| 2.3 | 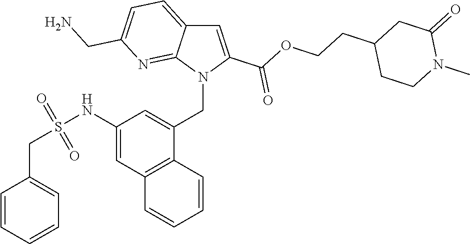 [SMILES: CN1CCC(CCOC(=O)c2cc3ccc(CN)nc3n2Cc2cc(NS(=O)(=O)Cc3ccccc3)cc3ccccc23)CC1=O] | 640.15 | A |
| 2.4 | 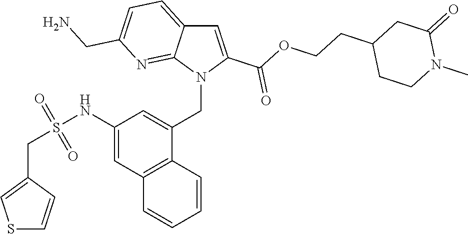 [SMILES: CN1CCC(CCOC(=O)c2cc3ccc(CN)nc3n2Cc2cc(NS(=O)(=O)Cc3ccsc3)cc3ccccc23)CC1=O] | 646.1 | A |
| 2.5 | 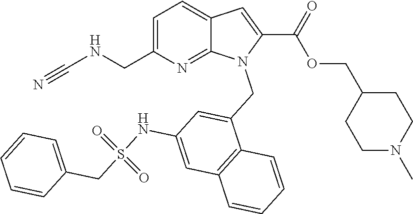 [SMILES: CN1CCC(COC(=O)c2cc3ccc(CNC#N)nc3n2Cc2cc(NS(=O)(=O)Cc3ccccc3)cc3ccccc23)CC1] | 655.4 | D |
| 2.6 | 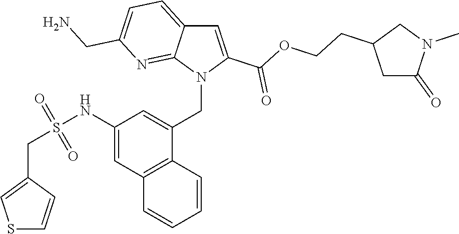 [SMILES: CN1CC(CCOC(=O)c2cc3ccc(CN)nc3n2Cc2cc(NS(=O)(=O)Cc3ccsc3)cc3ccccc23)CC1=O] | 632.4 | A |
| 2.7 | 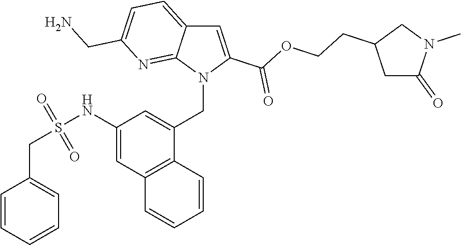 [SMILES: CN1CC(CCOC(=O)c2cc3ccc(CN)nc3n2Cc2cc(NS(=O)(=O)Cc3ccccc3)cc3ccccc23)CC1=O] | 626.4 | A |
| 2.8 | 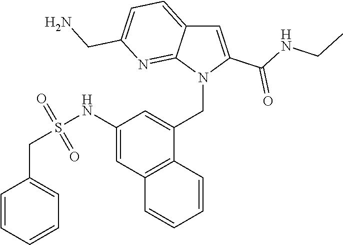 [SMILES: CCNC(=O)c1cc2ccc(CN)nc2n1Cc1cc(NS(=O)(=O)Cc2ccccc2)cc2ccccc12] | 527.95 | |
| 2.9 | 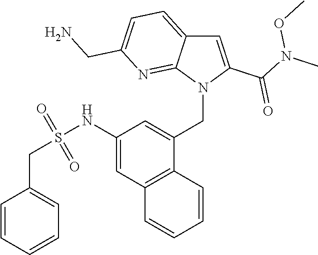 [SMILES: CON(C)C(=O)c1cc2ccc(CN)nc2n1Cc1cc(NS(=O)(=O)Cc2ccccc2)cc2ccccc12] | 544.3 | |
| 2.10 | 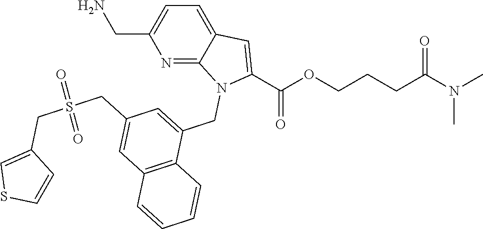 [SMILES: CN(C)C(=O)CCCOC(=O)c1cc2ccc(CN)nc2n1Cc1cc(CS(=O)(=O)Cc2ccsc2)cc2ccccc12] | 619.95 | A |
| 2.11 | 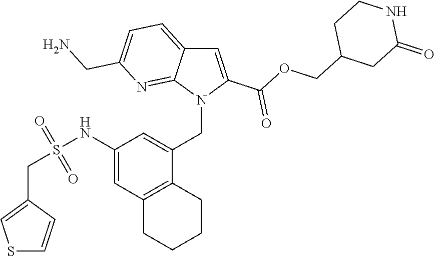 [SMILES: NCc1ccc2cc(C(=O)OCC3CCNC(=O)C3)n(Cc3cc(NS(=O)(=O)Cc4ccsc4)cc4c3CCCC4)c2n1] | 622.4 | A |
| 2.12 | 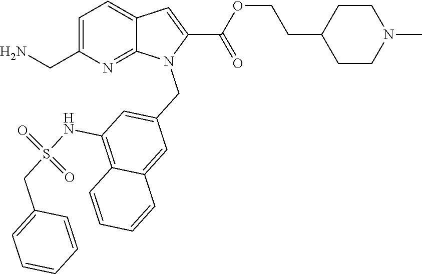 [SMILES: CN1CCC(CCOC(=O)c2cc3ccc(CN)nc3n2Cc2cc(NS(=O)(=O)Cc3ccccc3)c3ccccc3c2)CC1] | 626.4 | B |
| 2.13 | 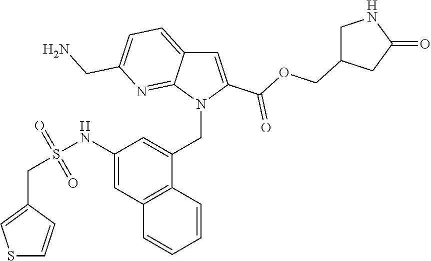 [SMILES: NCc1ccc2cc(C(=O)OCC3CNC(=O)C3)n(Cc3cc(NS(=O)(=O)Cc4ccsc4)cc4ccccc34)c2n1] | 603.95 | A |
| 2.14 | 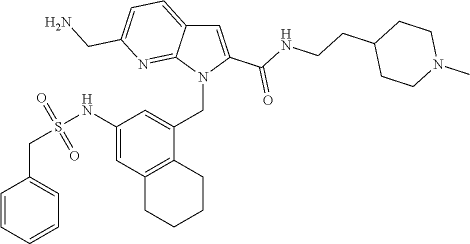 [SMILES: CN1CCC(CCNC(=O)c2cc3ccc(CN)nc3n2Cc2cc(NS(=O)(=O)Cc3ccccc3)cc3c2CCCC3)CC1] | 692.5 | A |
| 2.15 | 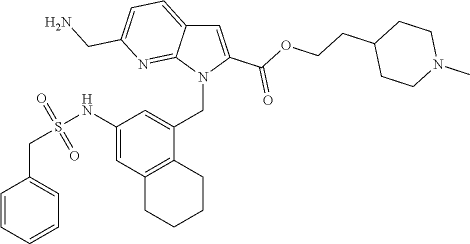 [SMILES: CN1CCC(CCOC(=O)c2cc3ccc(CN)nc3n2Cc2cc(NS(=O)(=O)Cc3ccccc3)cc3c2CCCC3)CC1] | 630.2 | A |
| 2.16 | 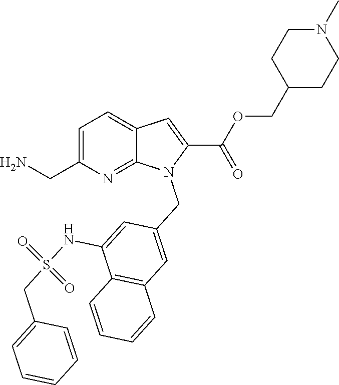 [SMILES: CN1CCC(COC(=O)c2cc3ccc(CN)nc3n2Cc2cc(NS(=O)(=O)Cc3ccccc3)c3ccccc3c2)CC1] | 612.4 | B |
| 2.17 | 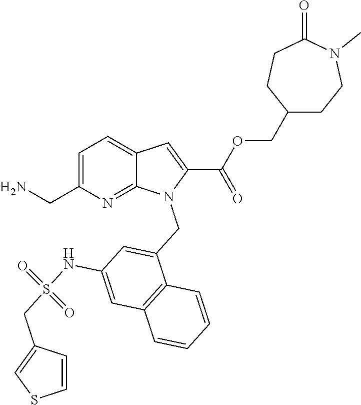 [SMILES: CN1CCC(COC(=O)c2cc3ccc(CN)nc3n2Cc2cc(NS(=O)(=O)Cc3ccsc3)cc3ccccc23)CCC1=O] | 646.4 | A |
| 2.18 | 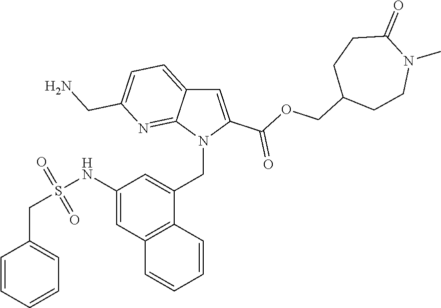 [SMILES: CN1CCC(COC(=O)c2cc3ccc(CN)nc3n2Cc2cc(NS(=O)(=O)Cc3ccccc3)cc3ccccc23)CCC1=O] | 640.4 | A |
| 2.19 | 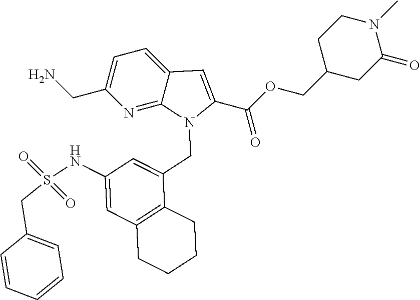 [SMILES: CN1CCC(COC(=O)c2cc3ccc(CN)nc3n2Cc2cc(NS(=O)(=O)Cc3ccccc3)cc3c2CCCC3)CC1=O] | 630.4 | A |
| 2.20 | 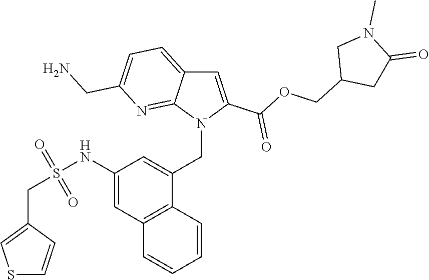 [SMILES: CN1CC(COC(=O)c2cc3ccc(CN)nc3n2Cc2cc(NS(=O)(=O)Cc3ccsc3)cc3ccccc23)CC1=O] | 618.1 | A |
| 2.21 | 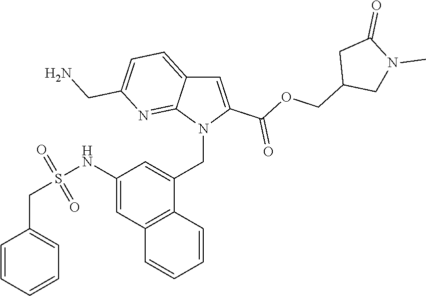 [SMILES: CN1CC(COC(=O)c2cc3ccc(CN)nc3n2Cc2cc(NS(=O)(=O)Cc3ccccc3)cc3ccccc23)CC1=O] | 612 | A |
| 2.22 | 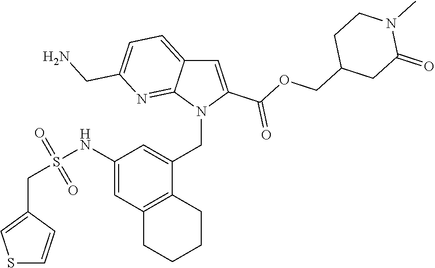 [SMILES: CN1CCC(COC(=O)c2cc3ccc(CN)nc3n2Cc2cc(NS(=O)(=O)Cc3ccsc3)cc3c2CCCC3)CC1=O] | 636.4 | A |
| 2.23 | 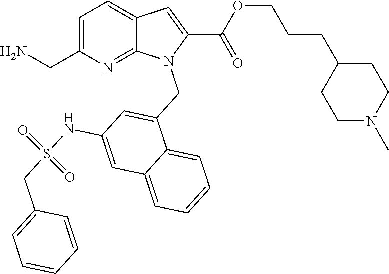 [SMILES: CN1CCC(CCCOC(=O)c2cc3ccc(CN)nc3n2Cc2cc(NS(=O)(=O)Cc3ccccc3)cc3ccccc23)CC1] | 640.2 | A |
| 2.24 | 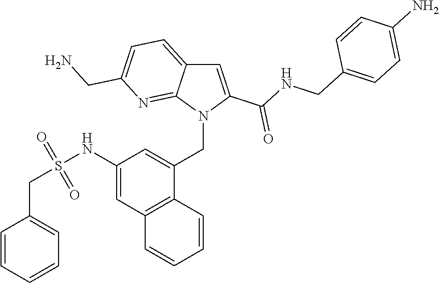 [SMILES: NCc1ccc2cc(C(=O)NCc3ccc(N)cc3)n(Cc3cc(NS(=O)(=O)Cc4ccccc4)cc4ccccc34)c2n1] | 605 | A |
| 2.25 | 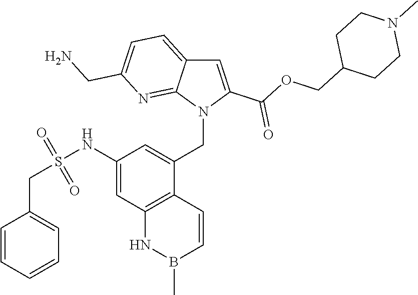 [SMILES: CB1C=Cc2c(Cn3c(C(=O)OCC4CCN(C)CC4)cc4ccc(CN)nc43)cc(NS(=O)(=O)Cc3ccccc3)cc2N1] | 627.4 | A |
| 2.26 | 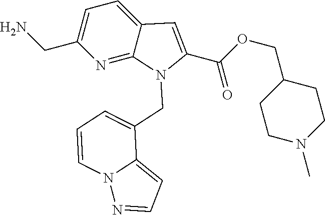 [SMILES: CN1CCC(COC(=O)c2cc3ccc(CN)nc3n2Cc2cccn3nccc23)CC1] | 433.05 | D |
| 2.27 | 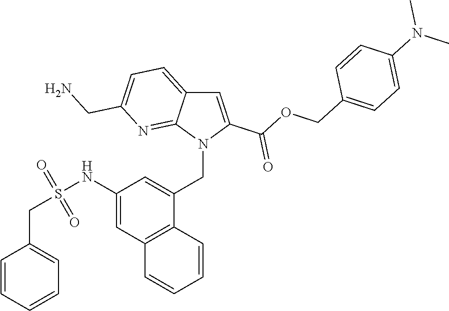 [SMILES: CN(C)c1ccc(COC(=O)c2cc3ccc(CN)nc3n2Cc2cc(NS(=O)(=O)Cc3ccccc3)cc3ccccc23)cc1] | 634.1 | B |
| 2.28 | 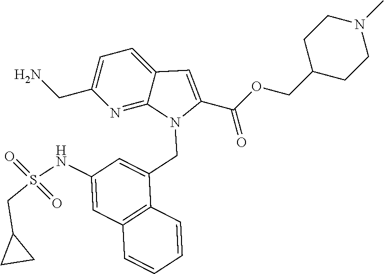 [SMILES: CN1CCC(COC(=O)c2cc3ccc(CN)nc3n2Cc2cc(NS(=O)(=O)CC3CC3)cc3ccccc23)CC1] | 576.3 | B |
| 2.29 | 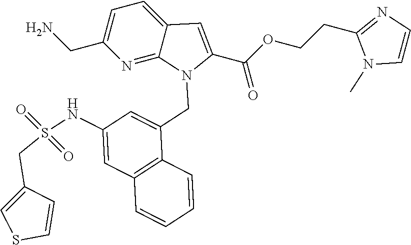 [SMILES: Cn1ccnc1CCOC(=O)c1cc2ccc(CN)nc2n1Cc1cc(NS(=O)(=O)Cc2ccsc2)cc2ccccc12] | 615.3 | A |
| 2.30 | 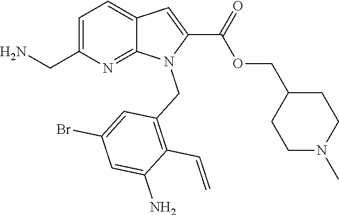 [SMILES: C=Cc1c(N)cc(Br)cc1Cn1c(C(=O)OCC2CCN(C)CC2)cc2ccc(CN)nc21] | 512.3 | D |
| 2.31 | 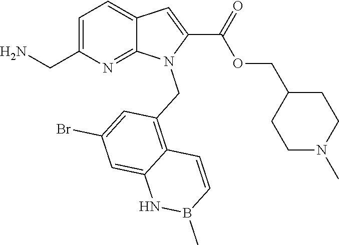 [SMILES: CB1C=Cc2c(Cn3c(C(=O)OCC4CCN(C)CC4)cc4ccc(CN)nc43)cc(Br)cc2N1] | 536.2 | C |
| 2.32 | 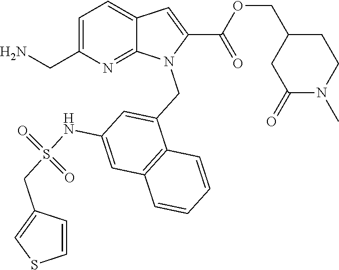 [SMILES: CN1CCC(COC(=O)c2cc3ccc(CN)nc3n2Cc2cc(NS(=O)(=O)Cc3ccsc3)cc3ccccc23)CC1=O] | 632.3 | A |
| 2.33 | 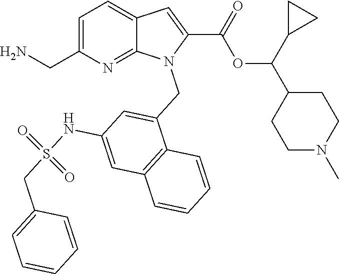 [SMILES: CN1CCC(C(OC(=O)c2cc3ccc(CN)nc3n2Cc2cc(NS(=O)(=O)Cc3ccccc3)cc3ccccc23)C2CC2)CC1] | 652.02 | A |
| 2.34 | 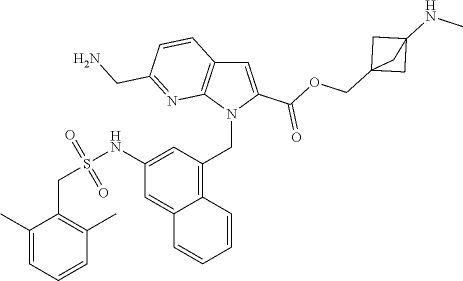 | 638.05 | A |
| 2.35 | 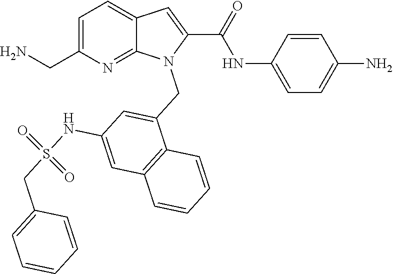 [SMILES: NCc1ccc2cc(C(=O)Nc3ccc(N)cc3)n(Cc3cc(NS(=O)(=O)Cc4ccccc4)cc4ccccc34)c2n1] | 591.2 | B |
| 2.36 | 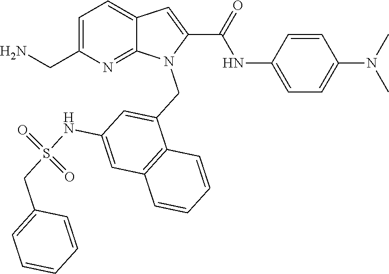 [SMILES: CN(C)c1ccc(NC(=O)c2cc3ccc(CN)nc3n2Cc2cc(NS(=O)(=O)Cc3ccccc3)cc3ccccc23)cc1] | 619 | B |
| 2.37 | 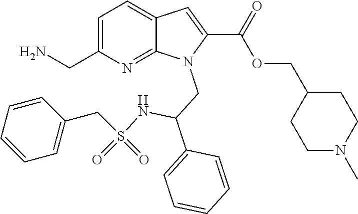 [SMILES: CN1CCC(COC(=O)c2cc3ccc(CN)nc3n2CC(NS(=O)(=O)Cc2ccccc2)c2ccccc2)CC1] | 576.4 | D |
| 2.38 | 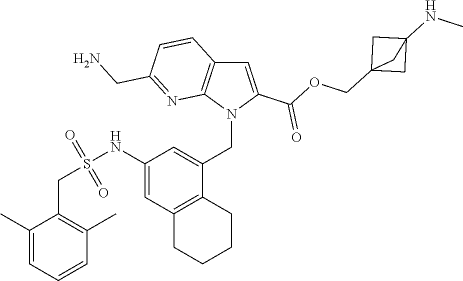 | 642.4 | A |
| 2.39 | 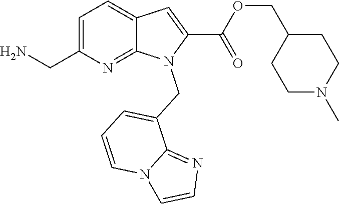 [SMILES: CN1CCC(COC(=O)c2cc3ccc(CN)nc3n2Cc2cccn3ccnc23)CC1] | 433.05 | D |
| 2.40 | 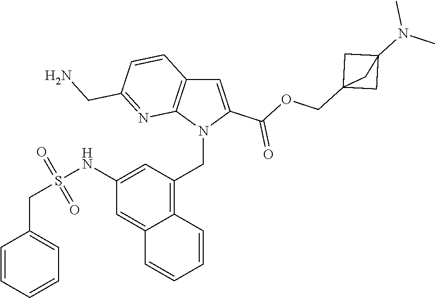 [SMILES: CN(C)C12CC(COC(=O)c3cc4ccc(CN)nc4n3Cc3cc(NS(=O)(=O)Cc4ccccc4)cc4ccccc34)(C1)C2] | 624.4 | A |
| 2.41 | 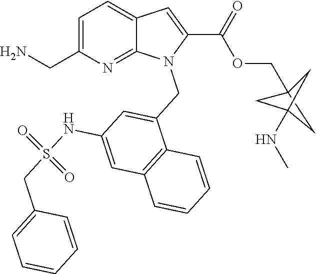 [SMILES: CNC12CC(COC(=O)c3cc4ccc(CN)nc4n3Cc3cc(NS(=O)(=O)Cc4ccccc4)cc4ccccc34)(C1)C2] | 610.4 | A |
| 2.42 | 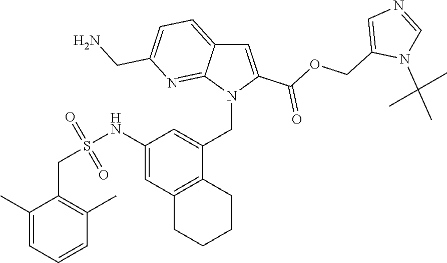 [SMILES: Cc1cccc(C)c1CS(=O)(=O)Nc1cc2c(c(Cn3c(C(=O)OCc4cncn4C(C)(C)C)cc4ccc(CN)nc43)c1)CCCC2] | 669.5 | B |
| 2.43 | 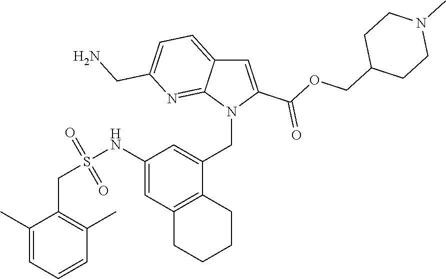 | 644.4 | A |
| 2.44 | 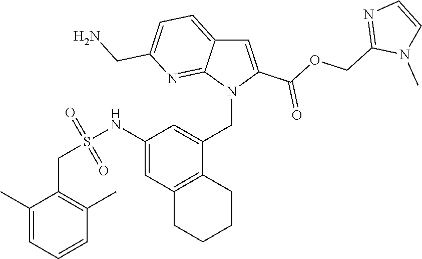 [SMILES: Cc1cccc(C)c1CS(=O)(=O)Nc1cc2c(c(Cn3c(C(=O)OCc4nccn4C)cc4ccc(CN)nc43)c1)CCCC2] | 627.5 | B |
| 2.45 | 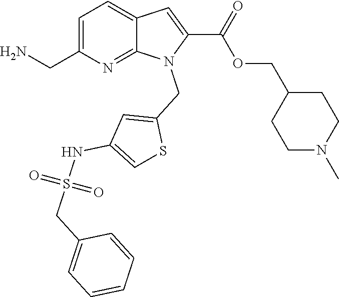 [SMILES: CN1CCC(COC(=O)c2cc3ccc(CN)nc3n2Cc2cc(NS(=O)(=O)Cc3ccccc3)cs2)CC1] | 568.3 | D |
| 2.46 | 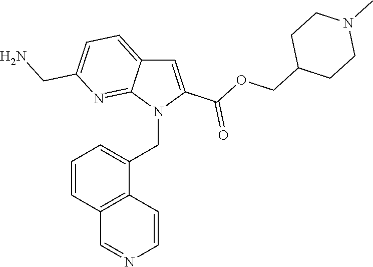 [SMILES: CN1CCC(COC(=O)c2cc3ccc(CN)nc3n2Cc2cccc3cnccc23)CC1] | 444.4 | D |
| 2.47 | 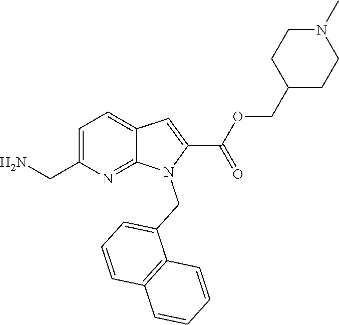 [SMILES: CN1CCC(COC(=O)c2cc3ccc(CN)nc3n2Cc2cccc3ccccc23)CC1] | 444.2 | C |
| 2.48 | 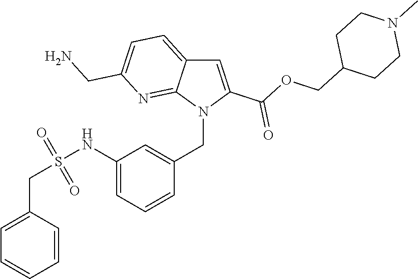 [SMILES: CN1CCC(COC(=O)c2cc3ccc(CN)nc3n2Cc2cccc(NS(=O)(=O)Cc3ccccc3)c2)CC1] | 562.3 | B |

TABLE C

| Cmpd # | Structure |
|---|---|
| 3.1 | 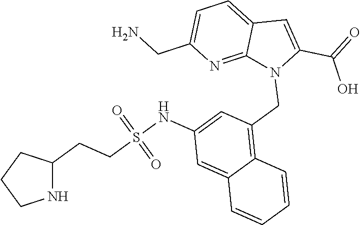 [SMILES: NCc1ccc2cc(C(=O)O)n(Cc3cc(NS(=O)(=O)CCC4CCCN4)cc4ccccc34)c2n1] |
| 3.2 | 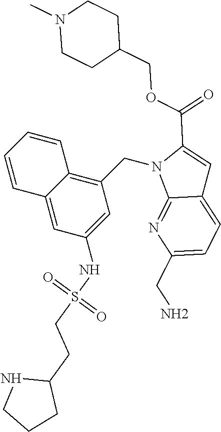 [SMILES: CN1CCC(COC(=O)c2cc3ccc(CN)nc3n2Cc2cc(NS(=O)(=O)CCC3CCCN3)cc3ccccc23)CC1] |
| 3.3 | 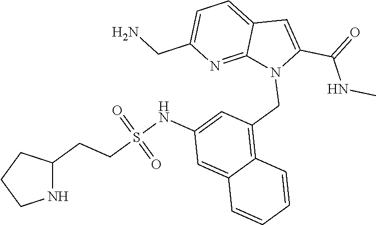 [SMILES: CNC(=O)c1cc2ccc(CN)nc2n1Cc1cc(NS(=O)(=O)CCC2CCCN2)cc2ccccc12] |
| 3.8 | 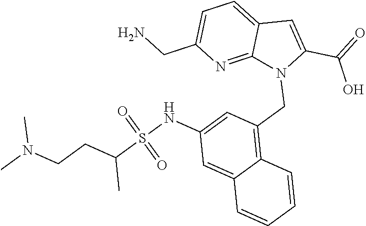 [SMILES: CC(CCN(C)C)S(=O)(=O)Nc1cc(Cn2c(C(=O)O)cc3ccc(CN)nc32)c2ccccc2c1] |
| 3.11 | 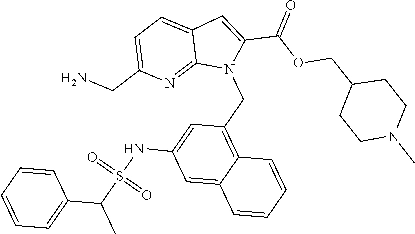 [SMILES: CC(c1ccccc1)S(=O)(=O)Nc1cc(Cn2c(C(=O)OCC3CCN(C)CC3)cc3ccc(CN)nc32)c2ccccc2c1] |
| 3.22 | 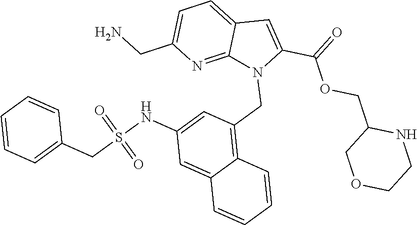 [SMILES: NCc1ccc2cc(C(=O)OCC3COCCN3)n(Cc3cc(NS(=O)(=O)Cc4ccccc4)cc4ccccc34)c2n1] |
| 3.23 | 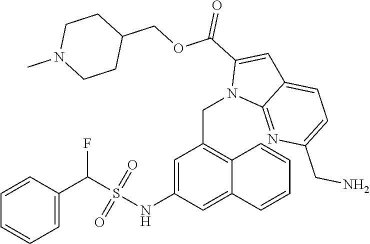 [SMILES: CN1CCC(COC(=O)c2cc3ccc(CN)nc3n2Cc2cc(NS(=O)(=O)C(F)c3ccccc3)cc3ccccc23)CC1] |
| 3.24 | 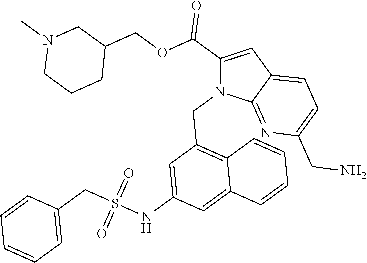 [SMILES: CN1CCCC(COC(=O)c2cc3ccc(CN)nc3n2Cc2cc(NS(=O)(=O)Cc3ccccc3)cc3ccccc23)C1] |
| 3.27 | 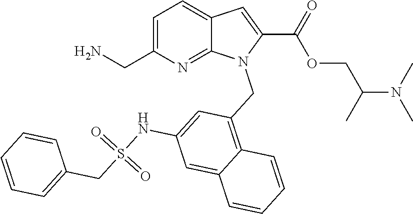 [SMILES: CC(COC(=O)c1cc2ccc(CN)nc2n1Cc1cc(NS(=O)(=O)Cc2ccccc2)cc2ccccc12)N(C)C] |
| 3.28 | 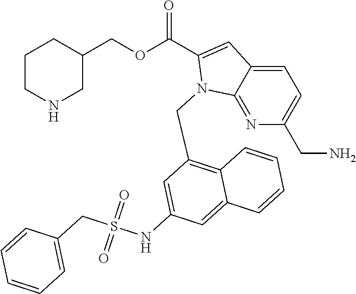 [SMILES: NCc1ccc2cc(C(=O)OCC3CCCNC3)n(Cc3cc(NS(=O)(=O)Cc4ccccc4)cc4ccccc34)c2n1] |
| 3.30 | 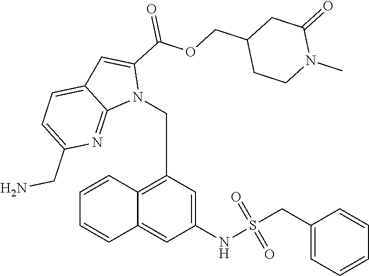 [SMILES: CN1CCC(COC(=O)c2cc3ccc(CN)nc3n2Cc2cc(NS(=O)(=O)Cc3ccccc3)cc3ccccc23)CC1=O] |
| 3.32 | 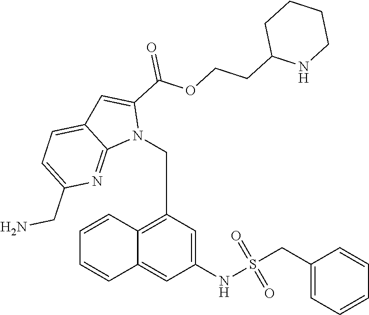 [SMILES: NCc1ccc2cc(C(=O)OCCC3CCCCN3)n(Cc3cc(NS(=O)(=O)Cc4ccccc4)cc4ccccc34)c2n1] |
| 3.36 | 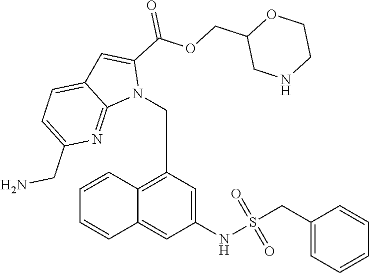 [SMILES: NCc1ccc2cc(C(=O)OCC3CNCCO3)n(Cc3cc(NS(=O)(=O)Cc4ccccc4)cc4ccccc34)c2n1] |
| 3.37 | 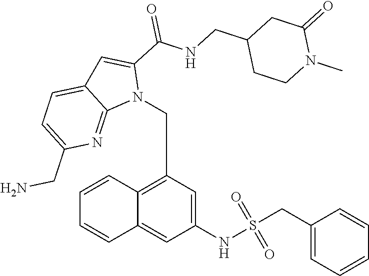 [SMILES: CN1CCC(CNC(=O)c2cc3ccc(CN)nc3n2Cc2cc(NS(=O)(=O)Cc3ccccc3)cc3ccccc23)CC1=O] |
| 3.41 | 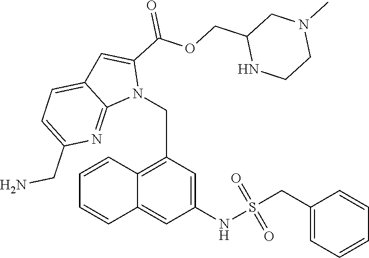 [SMILES: CN1CCNC(COC(=O)c2cc3ccc(CN)nc3n2Cc2cc(NS(=O)(=O)Cc3ccccc3)cc3ccccc23)C1] |
| 3.45 | 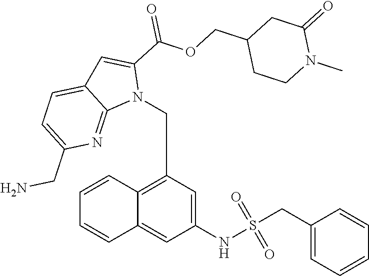 [SMILES: CN1CCC(COC(=O)c2cc3ccc(CN)nc3n2Cc2cc(NS(=O)(=O)Cc3ccccc3)cc3ccccc23)CC1=O] |
| 3.48 | 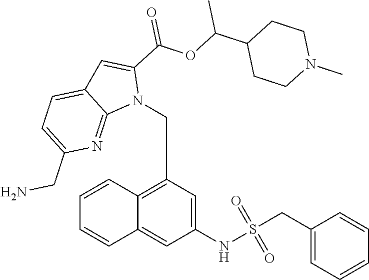 [SMILES: CC(OC(=O)c1cc2ccc(CN)nc2n1Cc1cc(NS(=O)(=O)Cc2ccccc2)cc2ccccc12)C1CCN(C)CC1] |
| 3.51 | 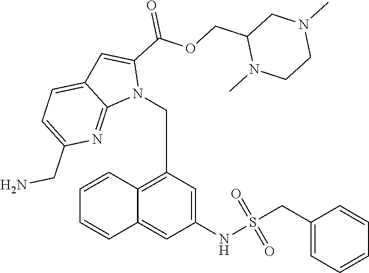 [SMILES: CN1CCN(C)C(COC(=O)c2cc3ccc(CN)nc3n2Cc2cc(NS(=O)(=O)Cc3ccccc3)cc3ccccc23)C1] |
| 3.54 | 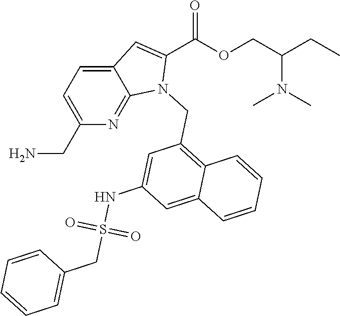 [SMILES: CCC(COC(=O)c1cc2ccc(CN)nc2n1Cc1cc(NS(=O)(=O)Cc2ccccc2)cc2ccccc12)N(C)C] |
| 3.63 | 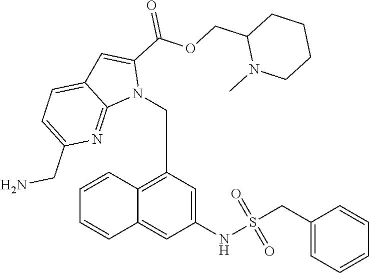 [SMILES: CN1CCCCC1COC(=O)c1cc2ccc(CN)nc2n1Cc1cc(NS(=O)(=O)Cc2ccccc2)cc2ccccc12] |
| 3.64 | 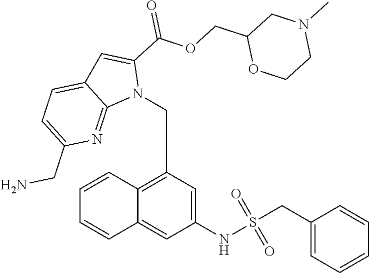 [SMILES: CN1CCOC(COC(=O)c2cc3ccc(CN)nc3n2Cc2cc(NS(=O)(=O)Cc3ccccc3)cc3ccccc23)C1] |
| 3.72 | 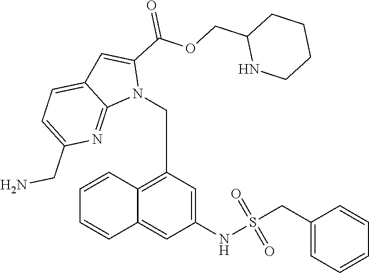 [SMILES: NCc1ccc2cc(C(=O)OCC3CCCCN3)n(Cc3cc(NS(=O)(=O)Cc4ccccc4)cc4ccccc34)c2n1] |
| 3.73 | 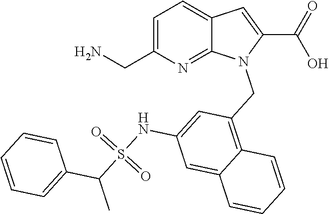 [SMILES: CC(c1ccccc1)S(=O)(=O)Nc1cc(Cn2c(C(=O)O)cc3ccc(CN)nc32)c2ccccc2c1] |
| 3.89 | 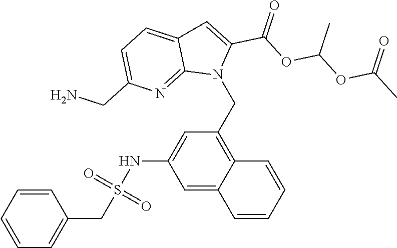 [SMILES: CC(=O)OC(C)OC(=O)c1cc2ccc(CN)nc2n1Cc1cc(NS(=O)(=O)Cc2ccccc2)cc2ccccc12] |
| 3.98 | 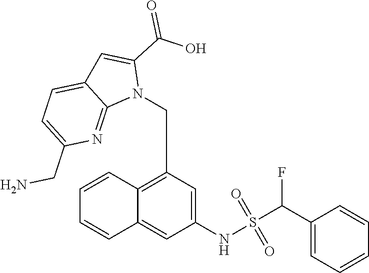 [SMILES: NCc1ccc2cc(C(=O)O)n(Cc3cc(NS(=O)(=O)C(F)c4ccccc4)cc4ccccc34)c2n1] |
| 3.108 | 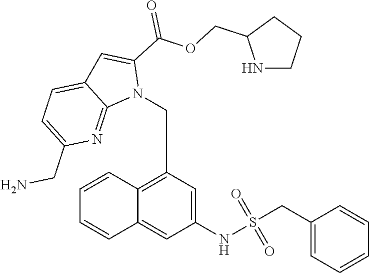 [SMILES: NCc1ccc2cc(C(=O)OCC3CCCN3)n(Cc3cc(NS(=O)(=O)Cc4ccccc4)cc4ccccc34)c2n1] |
| 3.118 | 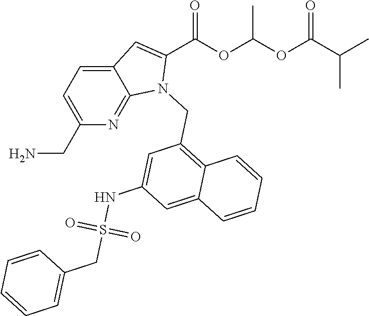 [SMILES: CC(OC(=O)c1cc2ccc(CN)nc2n1Cc1cc(NS(=O)(=O)Cc2ccccc2)cc2ccccc12)OC(=O)C(C)C] |
| 3.119 | 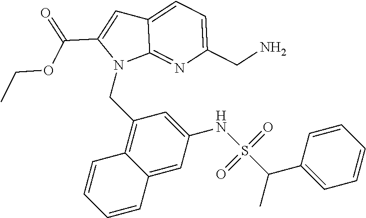 [SMILES: CCOC(=O)c1cc2ccc(CN)nc2n1Cc1cc(NS(=O)(=O)C(C)c2ccccc2)cc2ccccc12] |
| 3.124 | 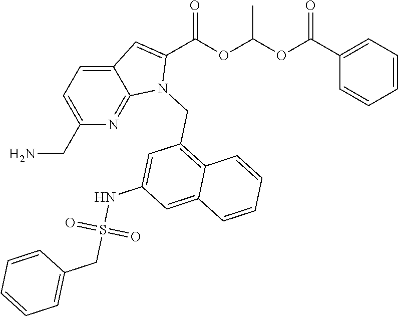 [SMILES: CC(OC(=O)c1ccccc1)OC(=O)c1cc2ccc(CN)nc2n1Cc1cc(NS(=O)(=O)Cc2ccccc2)cc2ccccc12] |
| 3.132 | 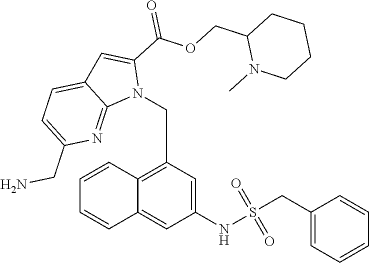 [SMILES: CN1CCCCC1COC(=O)c1cc2ccc(CN)nc2n1Cc1cc(NS(=O)(=O)Cc2ccccc2)cc2ccccc12] |
| 3.138 | 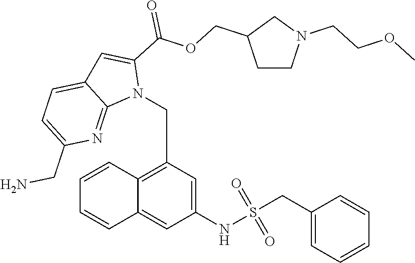 [SMILES: COCCN1CCC(COC(=O)c2cc3ccc(CN)nc3n2Cc2cc(NS(=O)(=O)Cc3ccccc3)cc3ccccc23)C1] |
| 3.143 | 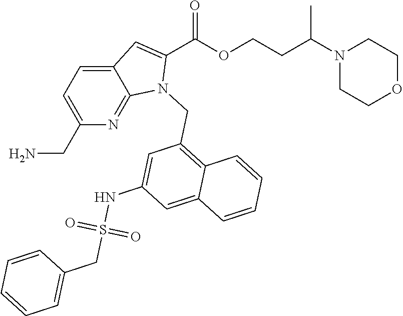 [SMILES: CC(CCOC(=O)c1cc2ccc(CN)nc2n1Cc1cc(NS(=O)(=O)Cc2ccccc2)cc2ccccc12)N1CCOCC1] |
| 3.144 | 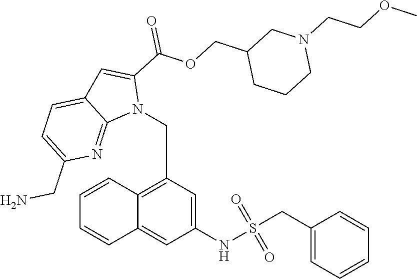 [SMILES: COCCN1CCCC(COC(=O)c2cc3ccc(CN)nc3n2Cc2cc(NS(=O)(=O)Cc3ccccc3)cc3ccccc23)C1] |
| 3.145 | 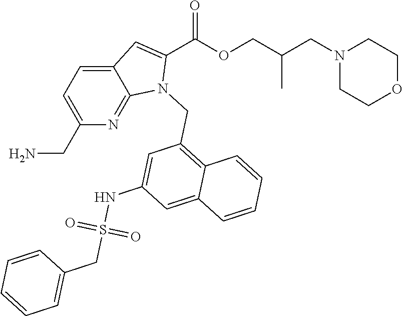 [SMILES: CC(COC(=O)c1cc2ccc(CN)nc2n1Cc1cc(NS(=O)(=O)Cc2ccccc2)cc2ccccc12)CN1CCOCC1] |
| 3.158 | 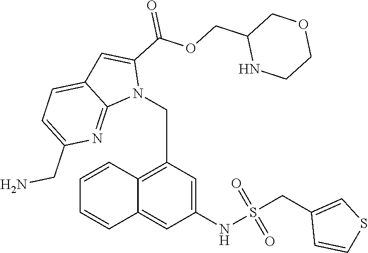 [SMILES: NCc1ccc2cc(C(=O)OCC3COCCN3)n(Cc3cc(NS(=O)(=O)Cc4ccsc4)cc4ccccc34)c2n1] |
| 3.170 | 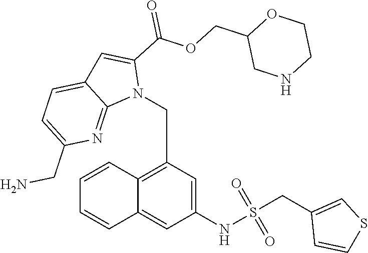 [SMILES: NCc1ccc2cc(C(=O)OCC3CNCCO3)n(Cc3cc(NS(=O)(=O)Cc4ccsc4)cc4ccccc34)c2n1] |
| 3.172 | 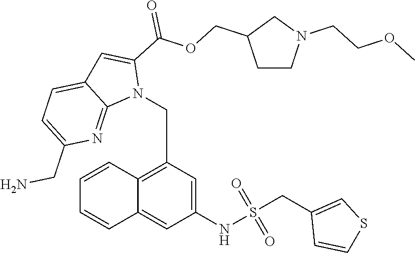 [SMILES: COCCN1CCC(COC(=O)c2cc3ccc(CN)nc3n2Cc2cc(NS(=O)(=O)Cc3ccsc3)cc3ccccc23)C1] |
| 3.182 | 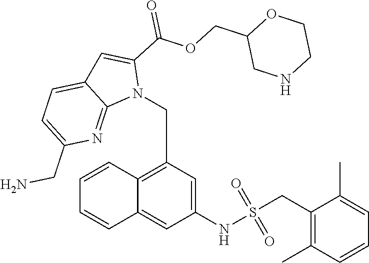 [SMILES: Cc1cccc(C)c1CS(=O)(=O)Nc1cc(Cn2c(C(=O)OCC3CNCCO3)cc3ccc(CN)nc32)c2ccccc2c1] |
| 3.189 | 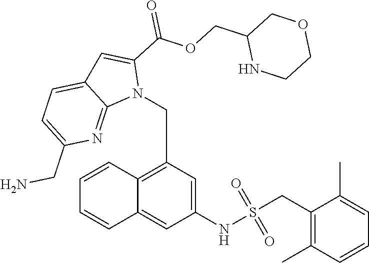 [SMILES: Cc1cccc(C)c1CS(=O)(=O)Nc1cc(Cn2c(C(=O)OCC3COCCN3)cc3ccc(CN)nc32)c2ccccc2c1] |
| 3.193 | 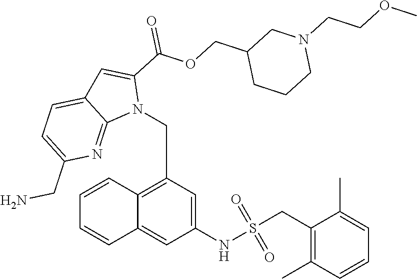 [SMILES: COCCN1CCCC(COC(=O)c2cc3ccc(CN)nc3n2Cc2cc(NS(=O)(=O)Cc3c(C)cccc3C)cc3ccccc23)C1] |
| 3.194 | 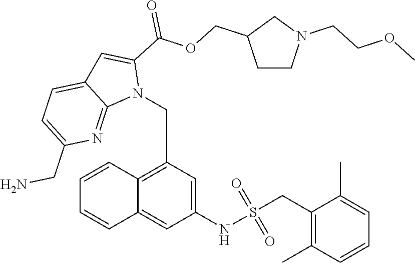 [SMILES: COCCN1CCC(COC(=O)c2cc3ccc(CN)nc3n2Cc2cc(NS(=O)(=O)Cc3c(C)cccc3C)cc3ccccc23)C1] |
| 3.208 | 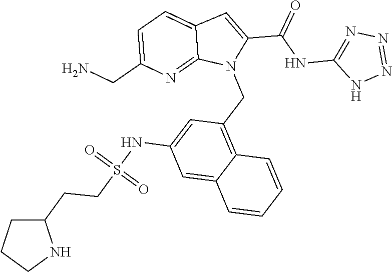 [SMILES: NCc1ccc2cc(C(=O)Nc3nnn[nH]3)n(Cc3cc(NS(=O)(=O)CCC4CCCN4)cc4ccccc34)c2n1] |
| 3.213 | 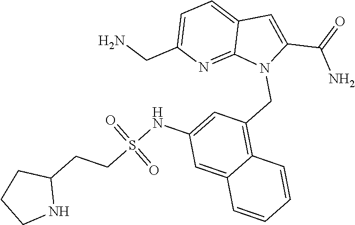 [SMILES: NCc1ccc2cc(C(N)=O)n(Cc3cc(NS(=O)(=O)CCC4CCCN4)cc4ccccc34)c2n1] |
| 3.217 | 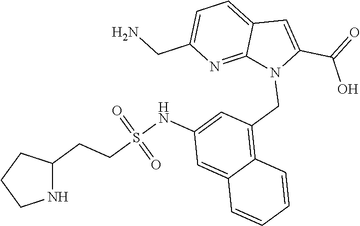 [SMILES: NCc1ccc2cc(C(=O)O)n(Cc3cc(NS(=O)(=O)CCC4CCCN4)cc4ccccc34)c2n1] |
| 3.224 | 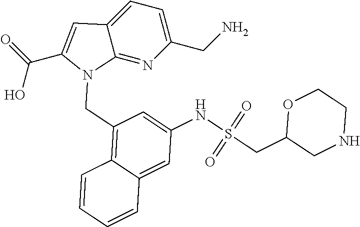 [SMILES: NCc1ccc2cc(C(=O)O)n(Cc3cc(NS(=O)(=O)CC4CNCCO4)cc4ccccc34)c2n1] |
| 3.235 | 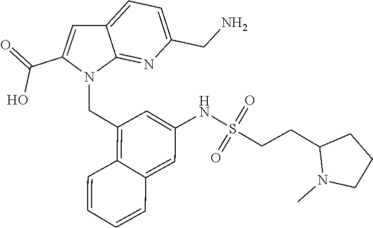 [SMILES: CN1CCCC1CCS(=O)(=O)Nc1cc(Cn2c(C(=O)O)cc3ccc(CN)nc32)c2ccccc2c1] |
| 3.254 | 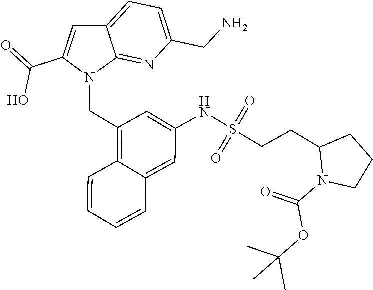 [SMILES: CC(C)(C)OC(=O)N1CCCC1CCS(=O)(=O)Nc1cc(Cn2c(C(=O)O)cc3ccc(CN)nc32)c2ccccc2c1] |
| 3.255 | 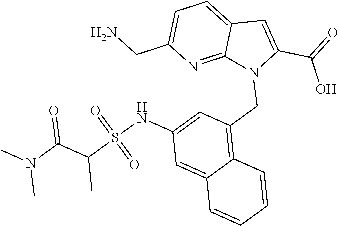 [SMILES: CC(C(=O)N(C)C)S(=O)(=O)Nc1cc(Cn2c(C(=O)O)cc3ccc(CN)nc32)c2ccccc2c1] |
| 3.257 | 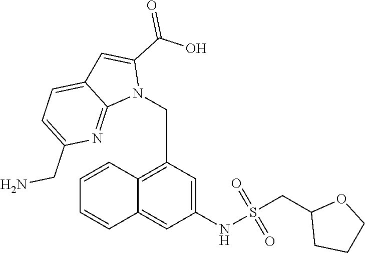 [SMILES: NCc1ccc2cc(C(=O)O)n(Cc3cc(NS(=O)(=O)CC4CCCO4)cc4ccccc34)c2n1] |
| 3.258 | 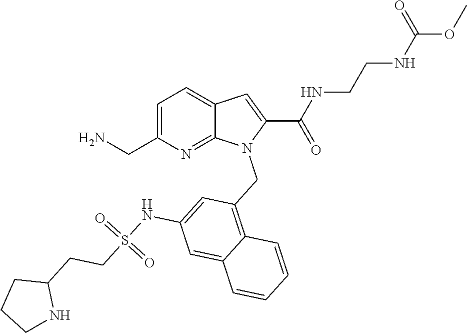 [SMILES: COC(=O)NCCNC(=O)c1cc2ccc(CN)nc2n1Cc1cc(NS(=O)(=O)CCC2CCCN2)cc2ccccc12] |
| 3.259 | 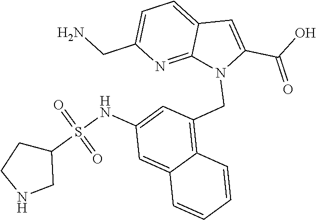 [SMILES: NCc1ccc2cc(C(=O)O)n(Cc3cc(NS(=O)(=O)C4CCNC4)cc4ccccc34)c2n1] |
| 3.260 | 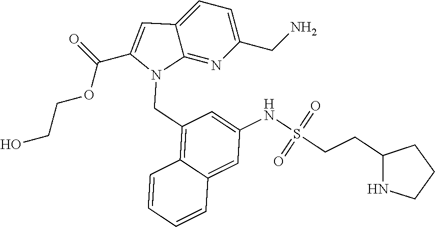 [SMILES: NCc1ccc2cc(C(=O)OCCO)n(Cc3cc(NS(=O)(=O)CCC4CCCN4)cc4ccccc34)c2n1] |
| 3.261 | 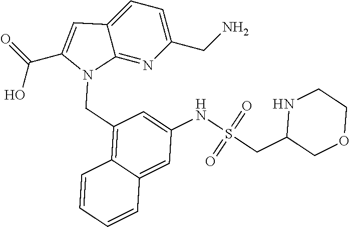 [SMILES: NCc1ccc2cc(C(=O)O)n(Cc3cc(NS(=O)(=O)CC4COCCN4)cc4ccccc34)c2n1] |
| 3.262 | 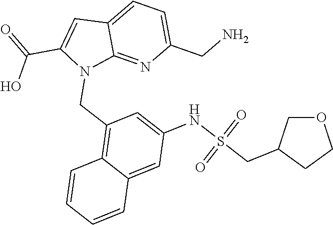 [SMILES: NCc1ccc2cc(C(=O)O)n(Cc3cc(NS(=O)(=O)CC4CCOC4)cc4ccccc34)c2n1] |
| 3.263 | 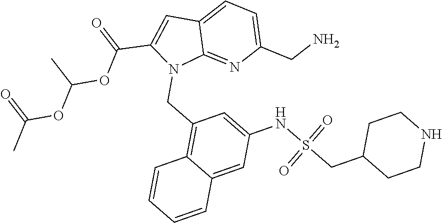 [SMILES: CC(=O)OC(C)OC(=O)c1cc2ccc(CN)nc2n1Cc1cc(NS(=O)(=O)CC2CCNCC2)cc2ccccc12] |
| 3.268 | 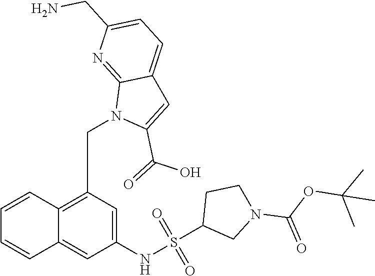 [SMILES: CC(C)(C)OC(=O)N1CCC(S(=O)(=O)Nc2cc(Cn3c(C(=O)O)cc4ccc(CN)nc43)c3ccccc3c2)C1] |
| 3.269 | 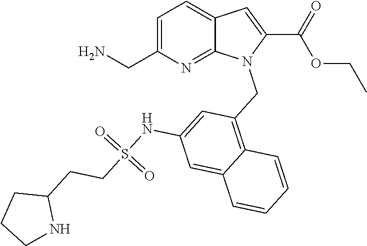 [SMILES: CCOC(=O)c1cc2ccc(CN)nc2n1Cc1cc(NS(=O)(=O)CCC2CCCN2)cc2ccccc12] |
| 3.270 | 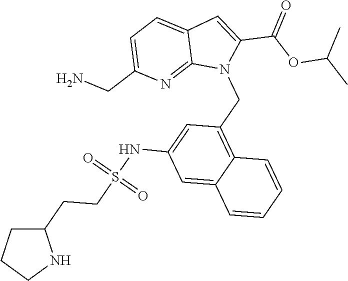 [SMILES: CC(C)OC(=O)c1cc2ccc(CN)nc2n1Cc1cc(NS(=O)(=O)CCC2CCCN2)cc2ccccc12] |
| 3.272 | 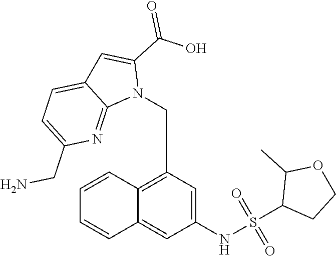 [SMILES: CC1OCCC1S(=O)(=O)Nc1cc(Cn2c(C(=O)O)cc3ccc(CN)nc32)c2ccccc2c1] |
| 3.273 | 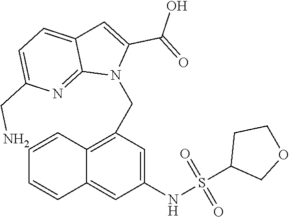 [SMILES: NCc1ccc2cc(C(=O)O)n(Cc3cc(NS(=O)(=O)C4CCOC4)cc4ccccc34)c2n1] |
| 3.275 | 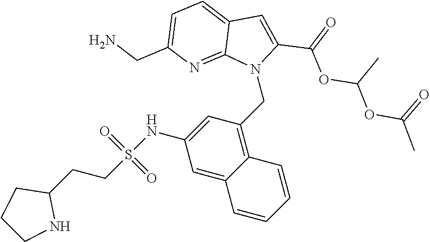 [SMILES: CC(=O)OC(C)OC(=O)c1cc2ccc(CN)nc2n1Cc1cc(NS(=O)(=O)CCC2CCCN2)cc2ccccc12] |
| 3.276 | 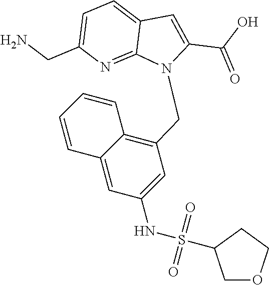 [SMILES: NCc1ccc2cc(C(=O)O)n(Cc3cc(NS(=O)(=O)C4CCOC4)cc4ccccc34)c2n1] |
| 3.277 | 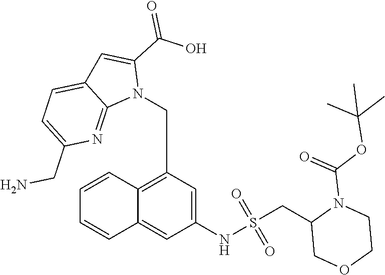 [SMILES: CC(C)(C)OC(=O)N1CCOCC1CS(=O)(=O)Nc1cc(Cn2c(C(=O)O)cc3ccc(CN)nc32)c2ccccc2c1] |
| 3.280 | 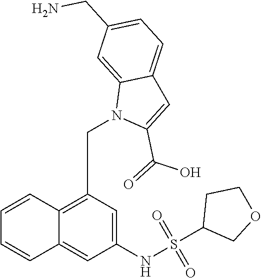 [SMILES: NCc1ccc2cc(C(=O)O)n(Cc3cc(NS(=O)(=O)C4CCOC4)cc4ccccc34)c2c1] |
| 3.281 | 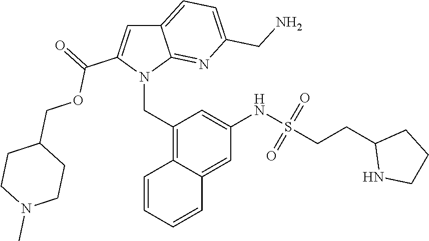 [SMILES: CN1CCC(COC(=O)c2cc3ccc(CN)nc3n2Cc2cc(NS(=O)(=O)CCC3CCCN3)cc3ccccc23)CC1] |
| 3.282 | 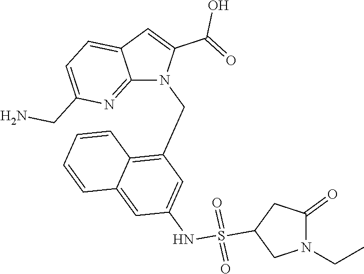 [SMILES: CCN1CC(S(=O)(=O)Nc2cc(Cn3c(C(=O)O)cc4ccc(CN)nc43)c3ccccc3c2)CC1=O] |
| 3.283 | 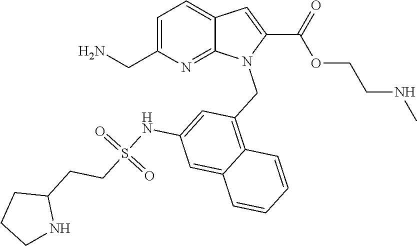 [SMILES: CNCCOC(=O)c1cc2ccc(CN)nc2n1Cc1cc(NS(=O)(=O)CCC2CCCN2)cc2ccccc12] |
| 3.284 | 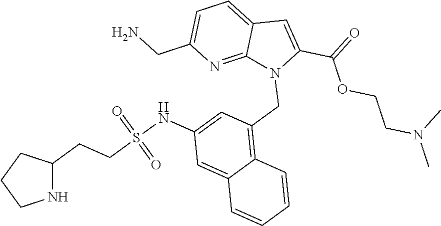 [SMILES: CN(C)CCOC(=O)c1cc2ccc(CN)nc2n1Cc1cc(NS(=O)(=O)CCC2CCCN2)cc2ccccc12] |
| 3.289 | 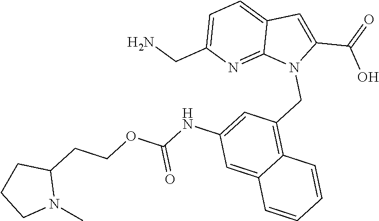 [SMILES: CN1CCCC1CCOC(=O)Nc1cc(Cn2c(C(=O)O)cc3ccc(CN)nc32)c2ccccc2c1] |
| 3.290 | 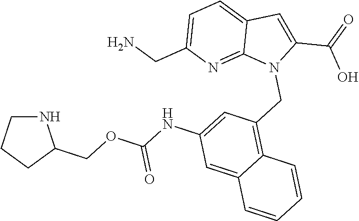 [SMILES: NCc1ccc2cc(C(=O)O)n(Cc3cc(NC(=O)OCC4CCCN4)cc4ccccc34)c2n1] |
| 3.295 | 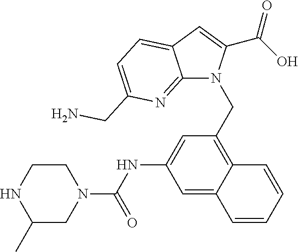 [SMILES: CC1CN(C(=O)Nc2cc(Cn3c(C(=O)O)cc4ccc(CN)nc43)c3ccccc3c2)CCN1] |
| 3.299 | 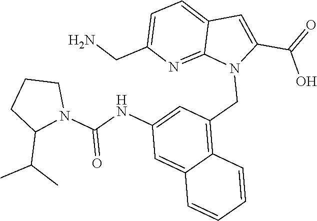 [SMILES: CC(C)C1CCCN1C(=O)Nc1cc(Cn2c(C(=O)O)cc3ccc(CN)nc32)c2ccccc2c1] |
| 3.301 | 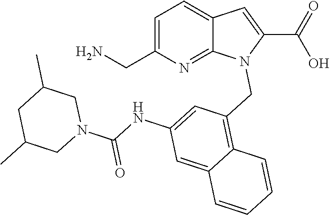 [SMILES: CC1CC(C)CN(C(=O)Nc2cc(Cn3c(C(=O)O)cc4ccc(CN)nc43)c3ccccc3c2)C1] |
| 3.302 | 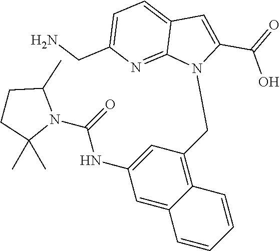 [SMILES: CC1CCC(C)(C)N1C(=O)Nc1cc(Cn2c(C(=O)O)cc3ccc(CN)nc32)c2ccccc2c1] |
| 3.303 | 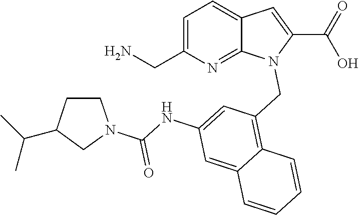 [SMILES: CC(C)C1CCN(C(=O)Nc2cc(Cn3c(C(=O)O)cc4ccc(CN)nc43)c3ccccc3c2)C1] |
| 3.304 | 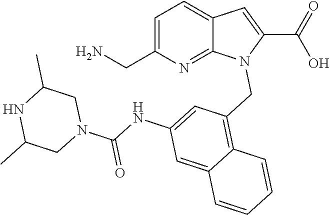 [SMILES: CC1CN(C(=O)Nc2cc(Cn3c(C(=O)O)cc4ccc(CN)nc43)c3ccccc3c2)CC(C)N1] |
| 3.305 | 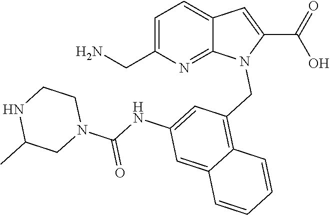 [SMILES: CC1CN(C(=O)Nc2cc(Cn3c(C(=O)O)cc4ccc(CN)nc43)c3ccccc3c2)CCN1] |
| 3.306 | 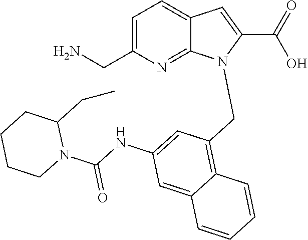 [SMILES: CCC1CCCCN1C(=O)Nc1cc(Cn2c(C(=O)O)cc3ccc(CN)nc32)c2ccccc2c1] |
| 3.307 | 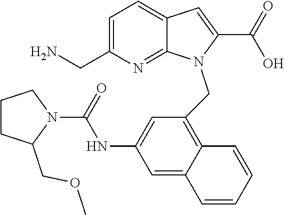 [SMILES: COCC1CCCN1C(=O)Nc1cc(Cn2c(C(=O)O)cc3ccc(CN)nc32)c2ccccc2c1] |
| 3.310 | 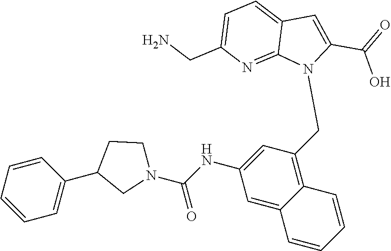 [SMILES: NCc1ccc2cc(C(=O)O)n(Cc3cc(NC(=O)N4CCC(c5ccccc5)C4)cc4ccccc34)c2n1] |
| 3.311 | 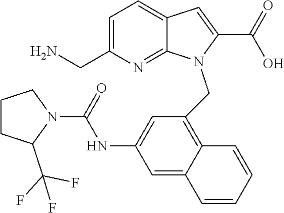 [SMILES: NCc1ccc2cc(C(=O)O)n(Cc3cc(NC(=O)N4CCCC4C(F)(F)F)cc4ccccc34)c2n1] |
| 3.313 | 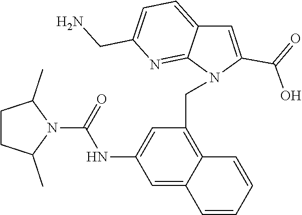 [SMILES: CC1CCC(C)N1C(=O)Nc1cc(Cn2c(C(=O)O)cc3ccc(CN)nc32)c2ccccc2c1] |
| 3.317 | 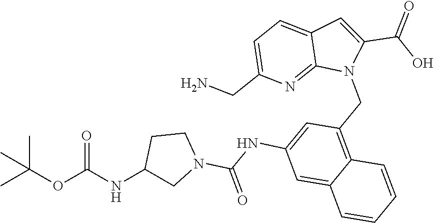 [SMILES: CC(C)(C)OC(=O)NC1CCN(C(=O)Nc2cc(Cn3c(C(=O)O)cc4ccc(CN)nc43)c3ccccc3c2)C1] |
| 3.318 | 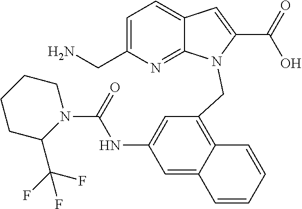 [SMILES: NCc1ccc2cc(C(=O)O)n(Cc3cc(NC(=O)N4CCCCC4C(F)(F)F)cc4ccccc34)c2n1] |
| 3.319 | 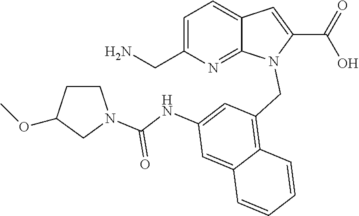 [SMILES: COC1CCN(C(=O)Nc2cc(Cn3c(C(=O)O)cc4ccc(CN)nc43)c3ccccc3c2)C1] |
| 3.321 | 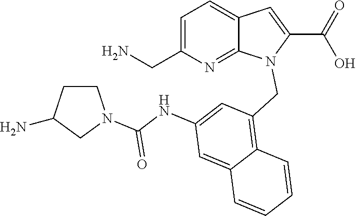 [SMILES: NCc1ccc2cc(C(=O)O)n(Cc3cc(NC(=O)N4CCC(N)C4)cc4ccccc34)c2n1] |
| 3.322 | 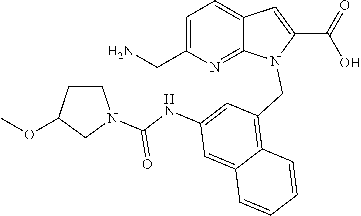 [SMILES: COC1CCN(C(=O)Nc2cc(Cn3c(C(=O)O)cc4ccc(CN)nc43)c3ccccc3c2)C1] |
| 3.323 | 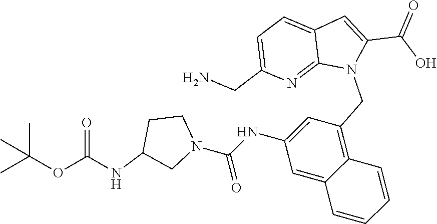 [SMILES: CC(C)(C)OC(=O)NC1CCN(C(=O)Nc2cc(Cn3c(C(=O)O)cc4ccc(CN)nc43)c3ccccc3c2)C1] |
| 3.324 | 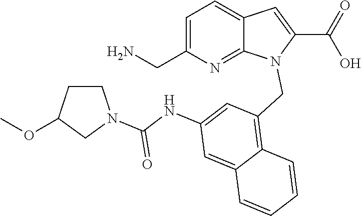 [SMILES: COC1CCN(C(=O)Nc2cc(Cn3c(C(=O)O)cc4ccc(CN)nc43)c3ccccc3c2)C1] |
| 3.325 | 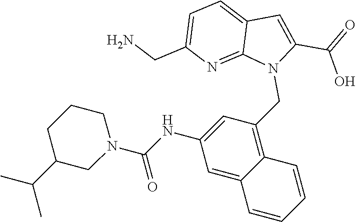 [SMILES: CC(C)C1CCCN(C(=O)Nc2cc(Cn3c(C(=O)O)cc4ccc(CN)nc43)c3ccccc3c2)C1] |
| 3.326 | 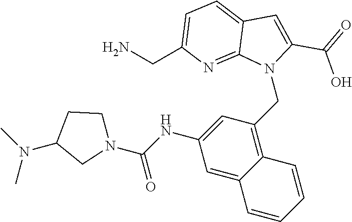 [SMILES: CN(C)C1CCN(C(=O)Nc2cc(Cn3c(C(=O)O)cc4ccc(CN)nc43)c3ccccc3c2)C1] |
| 3.328 | 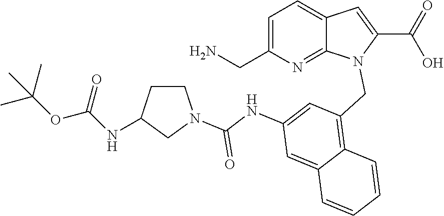 [SMILES: CC(C)(C)OC(=O)NC1CCN(C(=O)Nc2cc(Cn3c(C(=O)O)cc4ccc(CN)nc43)c3ccccc3c2)C1] |
| 3.329 | 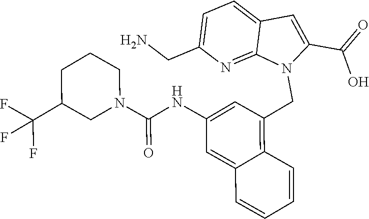 [SMILES: NCc1ccc2cc(C(=O)O)n(Cc3cc(NC(=O)N4CCCC(C(F)(F)F)C4)cc4ccccc34)c2n1] |
| 3.330 | 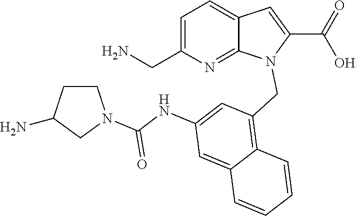 [SMILES: NCc1ccc2cc(C(=O)O)n(Cc3cc(NC(=O)N4CCC(N)C4)cc4ccccc34)c2n1] |
| 3.331 | 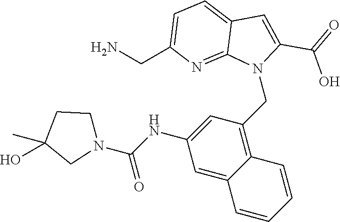 [SMILES: CC1(O)CCN(C(=O)Nc2cc(Cn3c(C(=O)O)cc4ccc(CN)nc43)c3ccccc3c2)C1] |
| 3.332 | 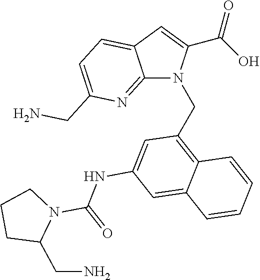 [SMILES: NCc1ccc2cc(C(=O)O)n(Cc3cc(NC(=O)N4CCCC4CN)cc4ccccc34)c2n1] |
| 3.334 | 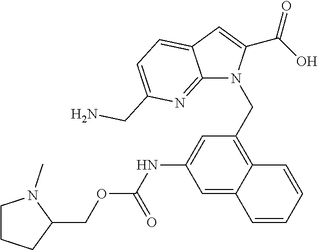 [SMILES: CN1CCCC1COC(=O)Nc1cc(Cn2c(C(=O)O)cc3ccc(CN)nc32)c2ccccc2c1] |
| 3.335 | 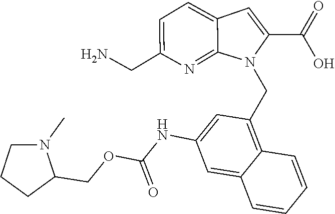 [SMILES: CN1CCCC1COC(=O)Nc1cc(Cn2c(C(=O)O)cc3ccc(CN)nc32)c2ccccc2c1] |
| 3.336 | 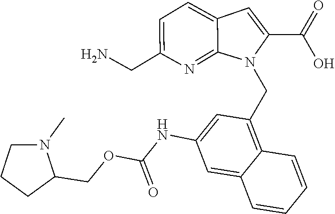 [SMILES: CN1CCCC1COC(=O)Nc1cc(Cn2c(C(=O)O)cc3ccc(CN)nc32)c2ccccc2c1] |
| 3.337 | 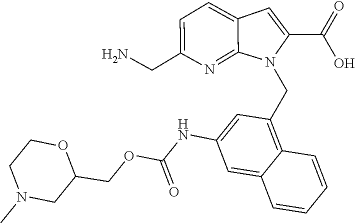 [SMILES: CN1CCOC(COC(=O)Nc2cc(Cn3c(C(=O)O)cc4ccc(CN)nc43)c3ccccc3c2)C1] |
| 3.339 | 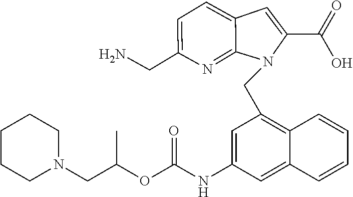 [SMILES: CC(CN1CCCCC1)OC(=O)Nc1cc(Cn2c(C(=O)O)cc3ccc(CN)nc32)c2ccccc2c1] |
| 3.340 | 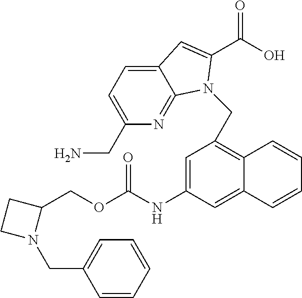 [SMILES: NCc1ccc2cc(C(=O)O)n(Cc3cc(NC(=O)OCC4CCN4Cc4ccccc4)cc4ccccc34)c2n1] |
| 3.341 | 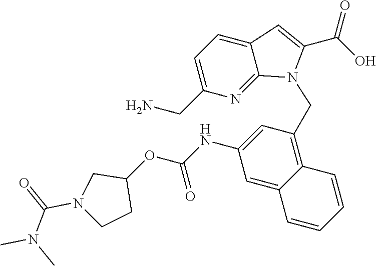 [SMILES: CN(C)C(=O)N1CCC(OC(=O)Nc2cc(Cn3c(C(=O)O)cc4ccc(CN)nc43)c3ccccc3c2)C1] |
| 3.342 | 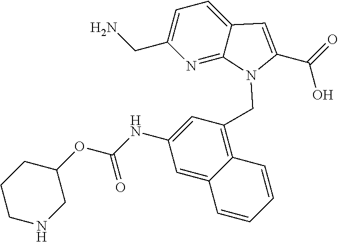 [SMILES: NCc1ccc2cc(C(=O)O)n(Cc3cc(NC(=O)OC4CCCNC4)cc4ccccc34)c2n1] |
| 3.343 | 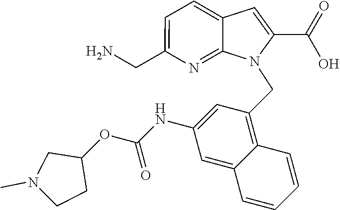 [SMILES: CN1CCC(OC(=O)Nc2cc(Cn3c(C(=O)O)cc4ccc(CN)nc43)c3ccccc3c2)C1] |
| 3.363 | 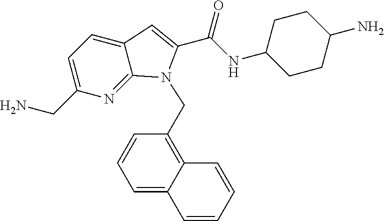 [SMILES: NCc1ccc2cc(C(=O)NC3CCC(N)CC3)n(Cc3cccc4ccccc34)c2n1] |
| 3.367 | 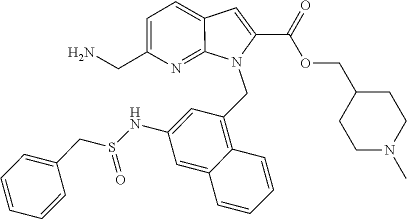 [SMILES: CN1CCC(COC(=O)c2cc3ccc(CN)nc3n2Cc2cc(NS(=O)Cc3ccccc3)cc3ccccc23)CC1] |
| 3.368 | 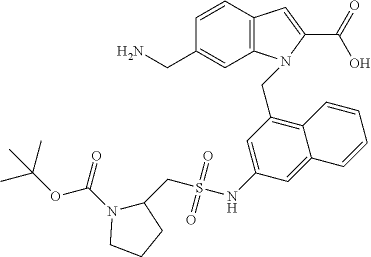 [SMILES: CC(C)(C)OC(=O)N1CCCC1CS(=O)(=O)Nc1cc(Cn2c(C(=O)O)cc3ccc(CN)cc32)c2ccccc2c1] |
| 3.369 | 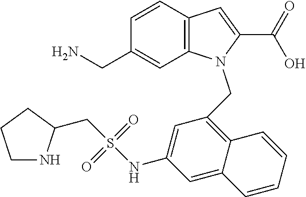 [SMILES: NCc1ccc2cc(C(=O)O)n(Cc3cc(NS(=O)(=O)CC4CCCN4)cc4ccccc34)c2c1] |
| 3.378 | 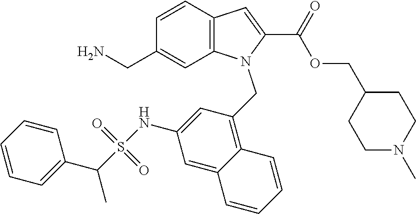 [SMILES: CC(c1ccccc1)S(=O)(=O)Nc1cc(Cn2c(C(=O)OCC3CCN(C)CC3)cc3ccc(CN)cc32)c2ccccc2c1] |
| 3.382 | 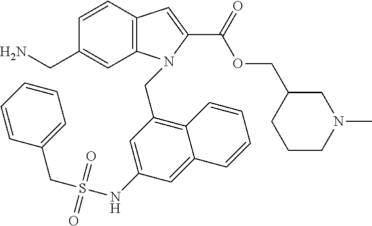 [SMILES: CN1CCCC(COC(=O)c2cc3ccc(CN)cc3n2Cc2cc(NS(=O)(=O)Cc3ccccc3)cc3ccccc23)C1] |
| 3.387 | 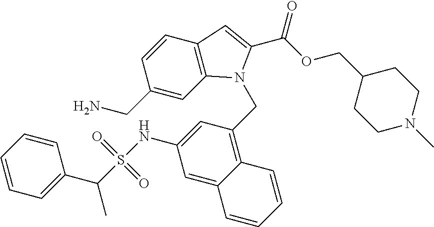 [SMILES: CC(c1ccccc1)S(=O)(=O)Nc1cc(Cn2c(C(=O)OCC3CCN(C)CC3)cc3ccc(CN)cc32)c2ccccc2c1] |
| 3.389 | 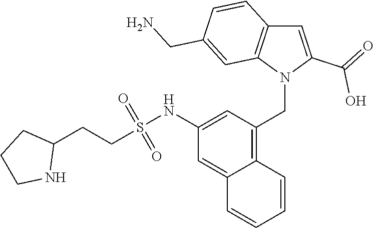 [SMILES: NCc1ccc2cc(C(=O)O)n(Cc3cc(NS(=O)(=O)CCC4CCCN4)cc4ccccc34)c2c1] |
| 3.391 | 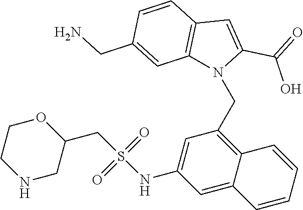 [SMILES: NCc1ccc2cc(C(=O)O)n(Cc3cc(NS(=O)(=O)CC4CNCCO4)cc4ccccc34)c2c1] |
| 3.395 | 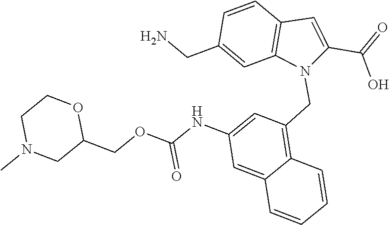 [SMILES: CN1CCOC(COC(=O)Nc2cc(Cn3c(C(=O)O)cc4ccc(CN)cc43)c3ccccc3c2)C1] |
| 3.398 | 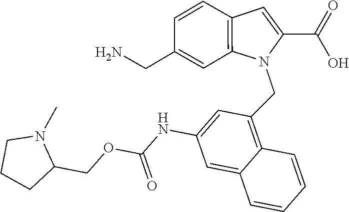 [SMILES: CN1CCCC1COC(=O)Nc1cc(Cn2c(C(=O)O)cc3ccc(CN)cc32)c2ccccc2c1] |
| 3.399 | 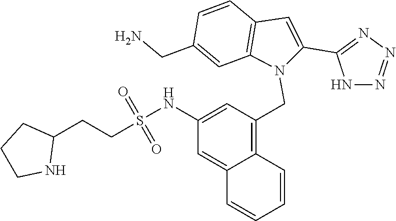 [SMILES: NCc1ccc2cc(-c3nnn[nH]3)n(Cc3cc(NS(=O)(=O)CCC4CCCN4)cc4ccccc34)c2c1] |

In some embodiments, the present disclosure provides a compound described in the examples below, or a pharmaceutically acceptable salt thereof.

In some embodiments, a compound of the disclosure is not:

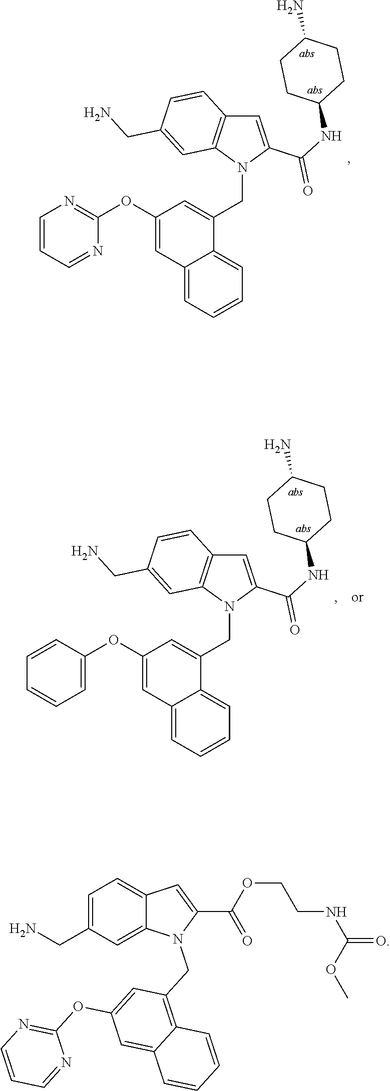 [SMILES: NCc1ccc2cc(C(=O)N[C@H]3CC[C@H](N)CC3)n(Cc3cc(Oc4ncccn4)cc4ccccc34)c2c1 ; NCc1ccc2cc(C(=O)N[C@H]3CC[C@H](N)CC3)n(Cc3cc(Oc4ccccc4)cc4ccccc34)c2c1 ; COC(=O)NCCOC(=O)c1cc2ccc(CN)cc2n1Cc1cc(Oc2ncccn2)cc2ccccc12]

In some embodiments, a compound of the disclosure is not:

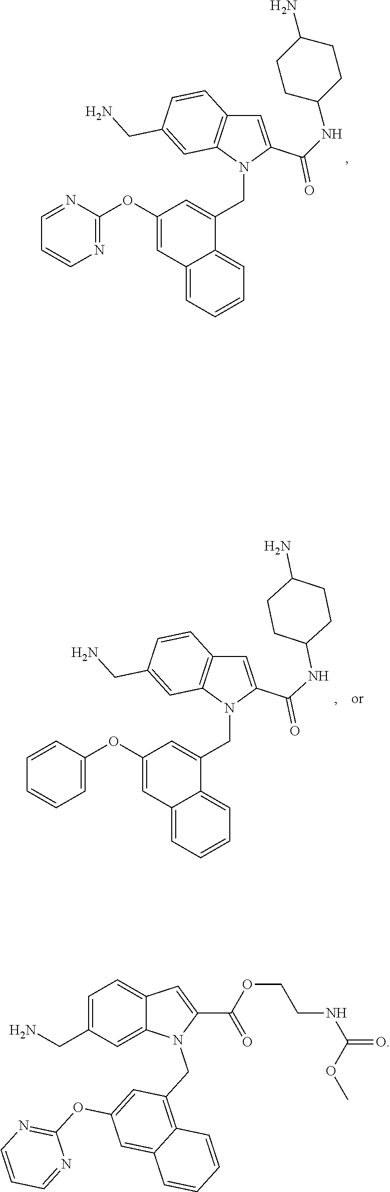 [SMILES: NCc1ccc2cc(C(=O)NC3CCC(N)CC3)n(Cc3cc(Oc4ncccn4)cc4ccccc34)c2c1 ; NCc1ccc2cc(C(=O)NC3CCC(N)CC3)n(Cc3cc(Oc4ccccc4)cc4ccccc34)c2c1 ; COC(=O)NCCOC(=O)c1cc2ccc(CN)cc2n1Cc1cc(Oc2ncccn2)cc2ccccc12]

### Uses, Formulation and Administration

The present disclosure also provides compositions comprising a compound disclosed herein, or pharmaceutically acceptable salts thereof, as well as methods of using the compounds and compositions provided herein. The methods herein should be understood to encompass method, use, Swiss-type claims, and the like (e.g., first medical use claims, purpose-limited composition claims, second medical use, and Swiss-type claim).

In one aspect, provided herein are pharmaceutical compositions comprising a compound as disclosed herein, or a pharmaceutically acceptable salt thereof, and a pharmaceutically acceptable excipient.

In another aspect, provided herein are pharmaceutical compositions comprising a compound as disclosed herein, or a pharmaceutically acceptable salt thereof, and a pharmaceutically acceptable carrier, adjuvant, or vehicle.

In a further aspect, provided herein are methods for treating a low hepcidin disorder, disease, and/or condition in a subject, comprising administering to the subject a compound as disclosed herein, or a pharmaceutically acceptable salt thereof, or a pharmaceutical composition as disclosed herein.

In one aspect, provided herein are methods for increasing hepcidin production by the liver in a subject, comprising administering to the subject a compound as disclosed herein, or a pharmaceutically acceptable salt thereof, or a pharmaceutical composition as disclosed herein.

In another aspect, provided herein are methods for treating an iron overload disorder, disease, and/or condition in a subject, comprising administering to the subject a compound as disclosed herein, or a pharmaceutically acceptable salt thereof, or a pharmaceutical composition as disclosed herein. In some embodiments, the iron overload disorder, disease, and/or condition is selected from the group consisting of hemochromatosis Type 1, 2a, 2b, and 3 (hemochromatosis, Hfe hemochromatosis (Type 1), juvenile hemochromatosis (types 2a and 2b), hepcidin deficiency, transfusional iron overload, African iron overload, and iron overload cardiomyopathy.

In a further aspect, provided herein are methods for treating an iron loading anemia in a subject, comprising administering to the subject a compound as disclosed herein, or a pharmaceutically acceptable salt thereof, or a pharmaceutical composition as disclosed herein. In some embodiments, the iron loading anemia is selected from the group consisting of beta thalassemia, HbE/thalassemia (thalassemia major, thalassemia intermedia, thalassemia minor, non-transfusion dependent thalassemia, transfusion-dependent thalassemia), alpha thalassemia, congenital dyserythropoietic anemias (Type I and Type II), pyruvate kinase deficiency, and myelodysplasia (such as myelodysplastic syndrome, and RARS SF3B1 associated MDS).

In one aspect, provided herein are methods method for treating a hematological disease, disorder, and/or condition in a subject, comprising administering to the subject a compound as disclosed herein, or a pharmaceutically acceptable salt thereof, or a pharmaceutical composition as disclosed herein. In some embodiments, the hematological disease, disorder, and/or condition is selected from the group consisting of sickle cell disease (such as sickle cell anemia), polycythemia vera, sideroblastic anemia, and bone marrow transplantation.

In a further aspect, provided herein are methods for treating a liver disease in a subject, comprising administering to the subject a compound as disclosed herein, or a pharmaceutically acceptable salt thereof, or a pharmaceutical composition as disclosed herein. In some embodiments, the liver disease is selected from the group consisting of Hepatitis B, Hepatitis C, alcoholic liver disease, cirrhosis of the liver, epahtocellular carcinoma, and non-alcoholic steatohepatitis (NASH).

In another aspect, provided herein are methods of treating a metabolic disease in a subject, comprising administering to the subject a compound as disclosed herein, or a pharmaceutically acceptable salt thereof, or a pharmaceutical composition as disclosed herein. In some embodiments, metabolic disease is selected from the group consisting of metabolic syndrome, insulin resistance, Type II diabetes, porphyria, porphyria cutanea tarda, Wilson's Disease. and acute iron overdose.

In a further aspect, provided herein are methods for treating a neurodegenerative disorder in a subject, comprising administering to the subject a compound as disclosed herein, or a pharmaceutically acceptable salt thereof, or a pharmaceutical composition as disclosed herein.

In another aspect, provided herein are methods for treating an infectious disease in a subject, comprising administering to the subject a compound as disclosed herein, or a pharmaceutically acceptable salt thereof, or a pharmaceutical composition as disclosed herein. In some embodiments, the infectious disease is a siderophilic infection.

In one aspect, provided herein are methods for treating polycythemia vera in a subject, comprising administering to the subject a compound as disclosed herein, or a pharmaceutically acceptable salt thereof, or a pharmaceutical composition as disclosed herein.

In some embodiments, the subject (i.e., the subject of the method) is a subject in need thereof.

In a further aspect, provided herein are methods of inhibiting matriptase 2, or a mutant thereof, in a biological sample, comprising contacting the sample with a compound as disclosed herein, or a pharmaceutically acceptable salt thereof, or a pharmaceutical composition as disclosed herein. In some embodiments, the contacting is in vivo or in vitro.

In a further aspect, provided herein are methods of inhibiting matriptase 2, or a mutant thereof, in a subject, comprising administering to the subject a compound as disclosed herein, or a pharmaceutically acceptable salt thereof, or a pharmaceutical composition as disclosed herein. In some embodiments, the contacting is in vivo or in vitro.

### Pharmaceutical Compositions

Embodiments described herein can be used in in pharmaceutical compositions and can be formulated by standard techniques using one or more physiologically acceptable carriers or excipients. In some embodiments, the present disclosure provides a composition comprising a compound of the present disclosure or a pharmaceutically acceptable derivative thereof and a pharmaceutically acceptable carrier, adjuvant, or vehicle. The amount of compound in compositions of the present disclosure is such that is effective to measurably inhibit Matriptase 2, or a mutant thereof, in a biological sample or in a patient. In certain embodiments, the amount of compound in compositions of the present disclosure is such that is effective to measurably inhibit Matriptase 2, or a mutant thereof, in a biological sample or in a patient. In certain embodiments, a composition of the present disclosure is formulated for administration to a patient in need of such composition. In some embodiments, a composition of the present disclosure is formulated for oral administration to a patient.

As used herein, the term “subject.” “individual” or “patient.” used interchangeably, means any animal, including preferably mammals, such as mice, rats, other rodents, rabbits, dogs, cats, swine, cattle, sheep, horses, or primates, such as most preferably humans.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of the present disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of the present disclosure that, upon administration to a recipient, is capable of providing, either directly or indirectly, a compound of the present disclosure or an inhibitorily active metabolite or residue thereof.

As used herein, the term “inhibitorily active metabolite or residue thereof” means that a metabolite or residue thereof is also an inhibitor of Matriptase 2, or a mutant thereof.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of the present disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed, including synthetic mono- or di-glycerides. Fatty acids, such as oleic acid and its glyceride derivatives, are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersants, such as carboxymethyl cellulose or similar dispersing agents that are commonly used in the formulation of pharmaceutically acceptable dosage forms, including emulsions and suspensions. Other commonly used surfactants, such as Tweens, Spans and other emulsifying agents or bioavailability enhancers which are commonly used in the manufacture of pharmaceutically acceptable solid, liquid, or other dosage forms, may also be used for the purposes of formulation.

Pharmaceutical compositions of the present disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutical compositions of the present disclosure may be administered in the form of suppositories for rectal administration. These can be prepared by mixing the agent with a suitable non-irritating excipient that is solid at room temperature but liquid at the rectal temperature and, therefore will melt in the rectum to release the drug. Such materials include cocoa butter, beeswax and polyethylene glycols.

Pharmaceutical compositions of the present disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutical compositions may be formulated in a suitable ointment containing the active component suspended or dissolved in one or more carriers. Carriers for topical administration of compounds of the present disclosure include, but are not limited to, mineral oil, liquid petrolatum, white petrolatum, propylene glycol, polyoxyethylene, polyoxypropylene compound, emulsifying wax and water. Alternatively, provided pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutical compositions may be formulated as micronized suspensions in isotonic, pH adjusted sterile saline, or, preferably, as solutions in isotonic. pH adjusted sterile saline, either with or without a preservative such as benzylalkonium chloride. Alternatively, for ophthalmic uses, the pharmaceutical compositions may be formulated in an ointment such as petrolatum.

Pharmaceutical compositions of the present disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Pharmaceutical compositions of the present disclosure are formulated for oral administration. Such formulations may be administered with or without food. In some embodiments, pharmaceutical compositions of the present disclosure are administered without food. In other embodiments, pharmaceutical compositions of the present disclosure are administered with food.

The amount of compounds of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

### Dosages

Compounds as described and provided for herein may be administered to a patient at therapeutically effective doses to prevent, treat, or control diseases and disorders mediated, in whole or in part, by a Matriptase 2 interaction described herein. Pharmaceutical compositions comprising the compound as described and provided herein may be administered to a patient in an amount sufficient to elicit an effective protective or therapeutic response in the patient. The dose will be determined by the efficacy of the particular compound employed and the condition of the subject, as well as the body weight or surface area of the area to be treated. The size of the dose also will be determined by the existence, nature, and extent of any adverse effects that accompany the administration of a particular compound or vector in a particular subject.

The amount and frequency of administration of the compound comprising the compound as described and provided herein prepared according to a method described herein and/or the pharmaceutically acceptable salts thereof can be regulated according to the judgment of the attending clinician considering such factors as age, condition and size of the patient as well as severity of the symptoms being treated. An ordinarily skilled physician or veterinarian can readily determine and prescribe the effective amount of the drug required to prevent, counter or arrest the progress of the condition. In general, it is contemplated that an effective amount would be from 0.001 mg/kg to 10 mg/kg body weight, and in particular from 0.01 mg/kg to 1 mg/kg body weight. More specifically, it is contemplated that an effective amount would be to continuously infuse by intravenous administration from 0.01 micrograms/kg body weight/min to 100 micrograms/kg body weight/min for a period of 12 hours to 14 days. It may be appropriate to administer the required dose as two, three, four or more sub-doses at appropriate intervals throughout the day. Sub-doses may be formulated as unit dosage forms, for example, containing 0.01 to 500 mg, and in particular 0.1 mg to 200 mg of active ingredient per unit dosage form.

In some embodiments, the pharmaceutical preparations as described and provided herein are in unit dosage forms. In such form, the preparation is subdivided into suitably sized unit doses containing appropriate quantities of the active component, e.g., an effective amount to achieve the desired purpose. The quantity of active compound in a unit dose of preparation may be varied or adjusted from about 0.01 mg to about 1000 mg, from about 0.01 mg to about 750 mg, from about 0.01 mg to about 500 mg, or from about 0.01 mg to about 250 mg, according to the particular application. The actual dosage employed may be varied depending upon the requirements of the patient and the severity of the condition being treated. Determination of the proper dosage regimen for a particular situation is within the skill of the art. For convenience, the total dosage may be divided and administered in portions during the day as required.

### Uses of Compounds and Pharmaceutical Compositions

Compounds and pharmaceutical compositions as described and provided for herein are generally useful for the inhibition of Matriptase 2, or a mutant thereof.

The activity of a compound utilized in the present disclosure as an inhibitor of Matriptase 2, or a mutant thereof, may be assayed in vitro, in vivo or in a cell line. In vitro assays include assays that determine inhibition of Matriptase 2, or a mutant thereof. Alternate in vitro assays quantitate the ability of the inhibitor to bind to Matriptase 2, or a mutant thereof. Detailed conditions for assaying a compound utilized in the present disclosure as an inhibitor of Matriptase 2, or a mutant thereof, are set forth in the Examples below.

As used herein, the terms “treatment,” “treat.” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

As used herein, the terms “low hepcidin” disorders, diseases, and/or conditions as used herein means any disease or other deleterious condition in which absolute or relative hepcidin deficiency is known to play a role, or in which an increase in hepcidin may be therapeutically useful.

Provided compounds as described herein are inhibitors of Matriptase 2, or a mutant thereof, and are therefore useful for treating low hepcidin disorders, diseases, and/or conditions. Accordingly, in certain embodiments, the present disclosure provides methods for treating a low hepcidin disorder, disease, and/or condition, comprising the step of administering to a patient in need thereof a compound of the present disclosure or pharmaceutically acceptable composition thereof.

Without wishing to be bound by any specific theory, inhibition of Matriptase-2 has been found to lead to increased hepcidin production by the liver. Accordingly, in some embodiments, the present disclosure provides methods for increasing hepcidin production by the liver in a patient, comprising the step of administering to the patient a compound of the present disclosure or pharmaceutically acceptable composition thereof. In some embodiments, the present disclosure provides methods for treating absolute and/or relative hepcidin deficiency in a patient, comprising the step of administering to the patient a compound of the present disclosure or pharmaceutically acceptable composition thereof. In some embodiments, the present disclosure provides methods for treating hepcidin underproduction in a patient, comprising the step of administering to the patient a compound of the present disclosure or pharmaceutically acceptable composition thereof. In some embodiments, the present disclosure provides methods for treating excess or increased iron absorption or accumulation in a patient, comprising the step of administering to the patient a compound of the present disclosure, or pharmaceutically acceptable composition thereof in order to increase hepcidin production by the liver. In some embodiments, the present disclosure provides methods for treating ineffective erythropoiesis in a patient, comprising the step of administering to the patient a compound of the present disclosure or pharmaceutically acceptable composition thereof.

In some embodiments, the present disclosure provides methods for treating one or more iron overload disorders, disease, and/or conditions, comprising the step of administering to a patient in need thereof a compound of the present disclosure or pharmaceutically acceptable composition thereof.

As used herein, the term “iron overload disorder, disease, and/or condition” refers to a condition, disease, or disorder associated with excessive iron levels or iron overload. Large amounts of free iron in the bloodstream can lead to cell damage, especially in the liver, heart and endocrine glands. The causes of excess iron may be genetic, for example, the iron excess may be caused by a genetic condition such as hemochromatosis type 1 (classical hemochromatosis), hemochromatosis type 2A or 2B (juvenile hemochromatosis), hemochromatosis type 3, African iron overload, neonatal hemochromatosis, aceruloplasminemia, or congenital atransferrinemia. Examples of non-genetic causes of iron excess include dietary iron overload (including African iron overload), transfusional iron overload (due to a blood transfusion given to patients with thalassaemia or other congenital hematological disorders), hemodialysis, chronic liver disease (such as hepatitis C, cirrhosis, non-alcoholic steatohepatitis), porphyria cutanea tarda, post-portacaval shunting, dysmetabolic overload syndrome, iron tablet overdose (such as that caused by consumption by children of iron tablets intended for adults), or any other cause of acute or chronic iron overload.

In some embodiments, an iron overload disorder, disease, and/or condition is Hemochromatosis Type 1. In some embodiments, an iron overload disorder, disease, and/or condition is Hemochromatosis Type 2a. In some embodiments, an iron overload disorder, disease, and/or condition is Hemochromatosis Type 2b. In some embodiments, an iron overload disorder, disease, and/or condition is Hemochromatosis Type 3.

In some embodiments, an iron overload disorder, disease, and/or condition is hepcidin deficiency. In some embodiments, an iron overload disorder, disease, and/or condition is Transfusional iron overload. In some embodiments, an iron overload disorder, disease, and/or condition is African iron overload. In some embodiments, an iron overload disorder, disease, and/or condition is Iron overload cardiomyopathy.

In some embodiments, the present disclosure provides methods for treating one or more iron loading anemia, comprising the step of administering to a patient in need thereof a compound of the present disclosure, or pharmaceutically acceptable composition thereof. In some embodiments, an Iron Loading Anemia is beta-thalassemia, HbE/beta-thalassemia, or other variants thereof, including but not limited to: thalassemia major, thalassemia intermedia, thalassemia minor, non-transfusion dependent thalassemia, and transfusion-dependent thalassemia. In some embodiments, an iron loading anemia is associated with, and/or caused by, alpha thalassemia. In some embodiments, an iron loading anemia is congenital dyserythropoietic anemia Type I and/or Type II. In some embodiments, an iron loading anemia is pyruvate kinase deficiency. In some embodiments, an iron loading anemia is myelodysplasia including but not limited to myelodysplastic syndrome (MDS), RARS and/or SF3B1 associated MDS.

In some embodiments, the present disclosure provides methods for treating one or more hematological diseases, disorders, and/or conditions, comprising the step of administering to a patient in need thereof a compound of the present disclosure or pharmaceutically acceptable composition thereof. In some embodiments, a hematological disease, disorder, and/or condition is sickle cell disease. In some embodiments, a hematological disease, disorder, and/or condition is sickle cell anemia. In some embodiments, a hematological disease, disorder, and/or condition is polycythemia vera. In some embodiments, a hematological disease, disorder, and/or condition is sideroblastic anemia. In some embodiments, a hematological disease, disorder, and/or condition is bone marrow transplantation.

In some embodiments, the present disclosure provides methods for treating one or more liver diseases, comprising the step of administering to a patient in need thereof a compound of the present disclosure or pharmaceutically acceptable composition thereof. In some embodiments, a liver disease is Hepatitis B. In some embodiments, a liver disease is Hepatitis C or other forms of viral hepatitis. In some embodiments, a liver disease is alcoholic liver disease. In some embodiments, a liver disease is cirrhosis of the liver. In some embodiments, a liver disease is hepatocellular carcinoma. In some embodiments, a liver disease is non-alcoholic steatohepatitis (NASH).

In some embodiments, the present disclosure provides methods for treating one or more metabolic disease, comprising the step of administering to a patient in need thereof a compound of the present disclosure, or pharmaceutically acceptable composition thereof. In some embodiments, a metabolic disease is metabolic syndrome. In some embodiments, a metabolic disease is insulin resistance. In some embodiments, a metabolic disease is Type II diabetes. In some embodiments, a metabolic disease is porphyria. In some embodiments, a metabolic disease is porphyria cutanea tarda. In some embodiments, a metabolic disease is Wilson's Disease. In some embodiments, a metabolic disease is acute iron overdose.

In some embodiments, the present disclosure provides methods for treating one or more neurodegenerative disorders, comprising the step of administering to a patient in need thereof a compound of the present disclosure, or pharmaceutically acceptable composition thereof. In some embodiments, a neurodegenerative disorder is selected from the group consisting of Huntington's Disease (HD); Parkinson's Disease (PD); amyotrophic lateral sclerosis (ALS); frontotemporal dementia (FTD); corticobasal degeneration (CBD); progressive supranuclear palsy (PSP); dementia with Lewy Bodies (DLB); and multiple sclerosis (MS).

In some embodiments, the present disclosure provides methods for treating one or more infectious diseases, comprising the step of administering to a patient in need thereof a compound of the present disclosure, or pharmaceutically acceptable composition thereof. In some embodiments, an infectious disease is a siderophilic infection.

In some embodiments, the present disclosure provides methods for treating polycythemia vera, comprising the step of administering to a patient in need thereof a compound of the present disclosure, or pharmaceutically acceptable composition thereof.

The compounds and compositions, according to the method of the present disclosure, may be administered using any amount and any route of administration effective for treating or lessening the severity of a low hepcidin disease, disorder, and/or condition, or any amount and any route of administration effective for increasing hepcidin production by the liver. The exact amount required will vary from subject to subject, depending on the species, age, and general condition of the subject, the severity of the disease or condition, the particular agent, its mode of administration, and the like. Compounds of the present disclosure are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present disclosure will be decided by the attending physician within the scope of sound medical judgment. The specific effective dose level for any particular patient or organism will depend upon a variety of factors, including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutical compositions of the present disclosure can be administered to humans and other animals orally, rectally, parenterally, intracisternally, intravaginally, intraperitoneally, topically (as by powders, ointments, or drops), buccally, as an oral or nasal spray, or the like, depending on the severity of the disease or disorder being treated. In certain embodiments, the compounds of the present disclosure may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water. Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions, which can be dissolved or dispersed in sterile water or other sterile injectable medium prior to use.

In order to prolong the effect of a compound of the present disclosure, it is often desirable to slow the absorption of the compound from a subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of the present disclosure with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose, or starch. Such dosage forms may also comprise, as is normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of the present disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of the present disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In some embodiments, the present disclosure relates to a method of inhibiting matriptase 2 activity, or a mutant thereof, in a biological sample comprising the step of contacting said biological sample with a compound of the present disclosure, or a composition comprising said compound.

The term “biological sample”, as used herein, includes, without limitation, cell cultures or extracts thereof, biopsied material obtained from a mammal or extracts thereof; and blood, saliva, urine, feces, semen, tears, or other body fluids or extracts thereof.

Depending upon the particular condition, or disease, to be treated, additional therapeutic agents that are normally administered to treat that condition, may also be present in the compositions of the present disclosure. As used herein, additional therapeutic agents that are normally administered to treat a particular disease, or condition, are known as “appropriate for the disease, or condition, being treated.”

In some embodiments, the present disclosure provides methods of treating a disclosed disease or condition comprising administering to a subject in need thereof an effective amount of a compound disclosed herein or a pharmaceutically acceptable salt thereof and co-administering simultaneously or sequentially an effective amount of one or more additional therapeutic agents. In some embodiments, the method includes co-administering one additional therapeutic agent. In some embodiments, the method includes co-administering two or more additional therapeutic agents. In some embodiments, the combination of the disclosed compound and the additional therapeutic agent or agents acts synergistically. In some embodiments, an additional therapeutic agent is an iron chelating compound, or a pharmaceutically acceptable salt thereof. In some embodiments, an iron chelating compound, or a pharmaceutically acceptable salt thereof, is selected from the group consisting of deferasirox, deferiprone and deferoxamine.

In some embodiments, the present disclosure provides methods of treating a disclosed disease or condition comprising administering to a subject in need thereof an effective amount of a compound disclosed herein or a pharmaceutically acceptable salt thereof, and an iron chelating compound or a pharmaceutically acceptable salt thereof. In some embodiments, a subject is a subject with iron overload. In some embodiments, a subject is a subject with cardiac iron overload or iron overload related cardiomyopathy. In some embodiments, an iron chelating compound, or a pharmaceutically acceptable salt thereof, is selected from the group consisting of deferasirox, deferiprone and deferoxamine.

### Combination Therapies

In some embodiments, the present disclosure provides methods for treating one or more iron overload disorders, diseases, and/or conditions, comprising the step of administering to a patient in need thereof a compound of the present disclosure, or pharmaceutically acceptable composition thereof, in combination with other drugs for the treatment of one or more iron overload disorders, diseases, and/or conditions.

In the combination therapies, the compound as described and provided for herein is co-administered with one or more drugs for the treatment of one or more iron overload disorders, diseases, and/or conditions to increase efficacy and to reduce side effects associated with high doses of these therapeutics.

The combination therapies described above have synergistic and additive therapeutic effects. An improvement in the drug therapeutic regimen can be described as the interaction of two or more agents so that their combined effect reduces the incidence of adverse event (AE) of either or both agents used in co-therapy. This reduction in the incidence of adverse effects can be a result of, e.g., administration of lower dosages of either or both agent used in the co-therapy. For example, if the effect of Drug A alone is 25% and has an adverse event incidence of 45% at labeled dose; and the effect of Drug B alone is 25% and has an adverse event incidence of 30% at labeled dose, but when the two drugs are combined at lower than labeled doses of each, if the overall effect is 35% (an improvement, but not synergistic or additive) and the adverse incidence rate is 20%, there is an improvement in the drug therapeutic regimen.

In some embodiments, the compounds described herein are administered as a mono-therapy. In some embodiments, the compounds described herein are administered as part of a combination therapy. For example, a compound may be used in combination with other drugs or therapies that are used in the treatment/prevention/suppression and/or amelioration of the diseases or conditions for which compounds are useful.

Such other drug(s) may be administered by a route and in an amount commonly used therefore, contemporaneously or sequentially with the compounds described herein. When a compound described herein is used contemporaneously with one or more other drugs, a pharmaceutical unit dosage form containing such other drugs in addition to the compound described herein may be employed. Accordingly, the pharmaceutical compositions include those that also contain one or more other active ingredients, in addition to the compounds described herein.

A subject or patient in whom administration of therapeutic compound is an effective therapeutic regimen for a disease or disorder is often a human, but can be any animal, including a laboratory animal in the context of a clinical trial or screening or activity experiment. Thus, as can be readily appreciated by one of ordinary skill in the art, the methods, compound and compositions are particularly suited to administration to any animal, such as a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

The following examples are merely illustrative and should not be construed as limiting the scope of the embodiments in any way as many variations and equivalents that are encompassed by these embodiments will become apparent to those skilled in the art upon reading the present disclosure.

In order that the embodiments disclosed herein may be more efficiently understood, examples are provided below. It should be understood that these examples are for illustrative purposes only and are not to be construed as limiting the embodiments in any manner.

Although the present embodiments have been described in connection with certain specific embodiments for instructional purposes, the present embodiments are not limited thereto. Accordingly, various modifications, adaptations, and combinations of various features of the described embodiments can be practiced without departing from the scope of the present disclosure as set forth in the claims. Furthermore, the following examples are illustrative, but not limiting, of the compounds, compositions and methods described herein. Other suitable modifications and adaptations of the variety of conditions and parameters normally encountered in therapy, synthesis, and other embodiments disclosed herein are within the spirit and scope of the embodiments. Other suitable modifications and adaptations known to those skilled in the art are within the scope of the following embodiments. Any and all journal articles, patent applications, issued patents, or other cited references are incorporated by reference in their entirety.

### EXAMPLES

### General Synthetic Methods

As depicted in the Examples below, in certain exemplary embodiments, compounds are prepared according to the following general procedures. It will be appreciated that, although the general methods depict the synthesis of certain compounds of the present disclosure, the following general methods, and other methods known to one of ordinary skill in the art, can be applied to all compounds and subclasses and species of each of these compounds, as described herein.

### General Modes of Preparation:

The general synthetic methods used in each General Procedure follow and include an illustration of a compound that was synthesized using the designated General Procedure. None of the specific conditions and reagents noted herein are to be construed as limiting the scope of the present disclosure and are provided for illustrative purposes only.

Compounds of the present disclosure may be made by synthetic chemical processes, examples of which are shown herein. It is meant to be understood that the order of the steps in the processes may be varied, that reagents, solvents, and reaction conditions may be substituted for those specifically mentioned, and those vulnerable moieties may be protected and deprotected, as necessary.

Unless otherwise stated, work-up includes distribution of the reaction mixture between the organic and aqueous phase indicated within parentheses, separation of layers and drying the organic layer over anhydrous sodium sulphate, filtration and distillation of the solvent under reduced pressure. Purification, unless otherwise mentioned, includes purification by silica gel chromatographic techniques, generally using ethyl acetate/petroleum ether mixture of a suitable polarity as the mobile phase.

The following abbreviations refer respectively to the definitions below:

ACN—Acetonitrile; br—Broad; ° C.—Degree Celsius; CHCl₃—Chloroform; CD₃OD—Deuterated Methanol; DMSO-d⁶—Deuterated dimethylsulfoxide; DCM—Dichloromethane; DIPEA—Diisopropylethylamine; DMF—N, N-Dimethylformamide; d—Doublet; dd—Doublet of doublet; EDC·HCl—1-(3-Dimethylaminopropyl)-3-ethylcarbodiimide hydrochloride; mg—Miligram; g—Gram; h—Hours; ¹H—Proton; HCl—Hydrochloric acid; HPLC—High-Performance Liquid Chromatography; H₂—Hydrogen; HOBt—1-Hydroxy benzotriazole; K₂CO₃—Potassium carbonate; LCMS—Liquid chromatography-mass spectroscopy; LiOH·H₂O—Lithium hydroxide monohydrate; M—Molar; MHz—Mega hertz (frequency); MeOH—Methanol; mL—MilliLiter; min—Minutes; mol—Moles; M⁺—Molecular ion; M—Multiplet; N₂—Nitrogen; NH₃—Ammonia; NBS—N-Bromosuccinimide; NCS—N-Chlorosuccinimide; NMR—Nuclear Magnetic Resonance; NaOH—Sodium Hydroxide; RT—Room temperature; s—Singlet; t—Triplet; TLC—Thin Layer Chromatography; TFA—Trifluoroacetic acid; TEA—Triethylamine; THF—Tetrahydrofuran; %—Percentage; μ—Micron; and δ—Delta; Zn—Zinc; mmol—millimoles.

Analysis for the compounds of the present disclosure unless mentioned, was conducted in the general methods well known to the person skilled in the art. Having described the present disclosure with reference to certain preferred embodiments, other embodiments will become apparent to one skilled in the art from consideration of the specification. The present disclosure is further defined by reference to the following examples, describing in detail the analysis of the compounds of the present disclosure.

LCMS data has been recorded in +ve mode unless otherwise mentioned.

It will be apparent to those skilled in the art that many modifications, both to materials and methods, may be practiced without departing from the scope of the present disclosure.

### General Synthetic Scheme 1 Intermediates:

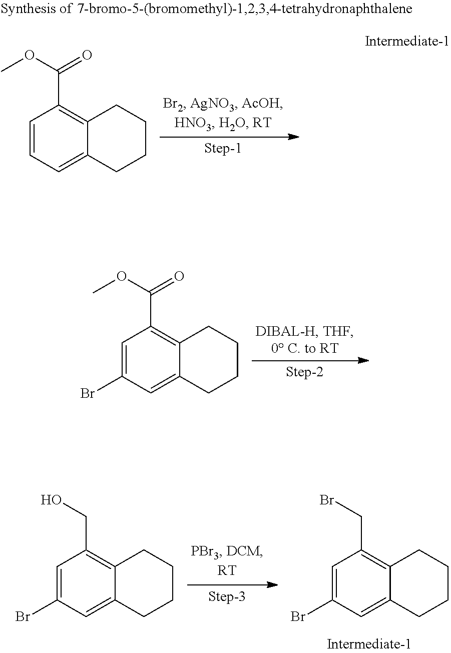

### Step-1: Synthesis of methyl 3-bromo-5,6,7,8-tetrahydronaphthalene-1-carboxylate

A stirred solution of methyl 5,6,7,8-tetrahydronaphthalene-1-carboxylate (4.2 g, 22 mmol) in acetic acid (37.5 mL) was cooled 0° C. and to the solution were added AgNO₃ (5.6 g, 33.1 mmol), bromine (1.76 g, 22 mmol) followed by Conc. nitric acid (1.6 mL). The reaction mixture was stirred at RT for 16 h, then quenched with Na₂S₂O₃ and extracted with ethyl acetate. The organic portion was dried over Na₂SO₄ and concentrated. The residue was purified by Combi flash chromatography using hexane as an eluent to afford the pure title compound (2.94 g, 49.4%).

¹HNMR (CDCl₃, 400 MHz): δ 1.80-1.90 (m, 4H), 2.80-2.95 (m, 2H), 3.00-3.10 (m, 2H), 3.90 (s, 3H), 7.35-7.36 (d, 1H), 7.45-7.55 (m, 1H), 7.75-7.85 (m, 1H).

### Step-2: Synthesis of (3-bromo-5,6,7,8-tetrahydronaphthalen-1-yl)methanol

To a stirred solution of methyl 3-bromo-5,6,7,8-tetrahydronaphthalene-1-carboxylate (3.5 g, 13 mmol) in DCM (35 mL), which was cooled to 0° C., was added DIBAL-H (3.67 g, 26 mmol) under nitrogen atmosphere. The resulting reaction mixture was stirred at RT for overnight, quenched by methanol at 0° C. and stirred for 30 minutes. The mixture was added Aqueous sodium potassium tartrate solution and stirred for about 1 h. The organic portion was dried over Na₂SO₄ and concentrated to yield the title compound (3.1 g, 98.8%).

¹HNMR (CDCl₃, 400 MHz): δ 1.82-1.90 (m, 4H), 2.81-2.96 (m, 5H), 4.64-4.65 (d, 2H), 7.15-7.25 (m, 1H), 7.30-7.45 (m, 1H), 7.47-7.60 (m, 1H).

### Step-3: Synthesis of 7-bromo-5-(bromomethyl)-1,2,3,4-tetrahydronaphthalene

A stirred solution of (3-bromo-5,6,7,8-tetrahydronaphthalen-1-yl)methanol (3.3 g, 13.9 mmol) in DCM (38 mL) was cooled to 0° C. and to the solution was added PBr₃ (1.8 g, 6.9 mmol) under nitrogen atmosphere. The reaction mixture was stirred at RT for overnight. Then the reaction mixture was quenched with Aqueous NaHCO₃, extracted with ethyl acetate, the organic portion was dried over Na₂SO₄ and concentrated to yield the title compound (3.69 g, 87.1%).

¹HNMR (CDCl₃, 400 MHz): δ 1.70-1.85 (m, 4H), 2.85-2.96 (m, 4H), 4.66 (s, 2H), 7.05-7.15 (m, 1H), 7.26-7.34 (m, 1H), 7.57-7.60 (m, 1H).

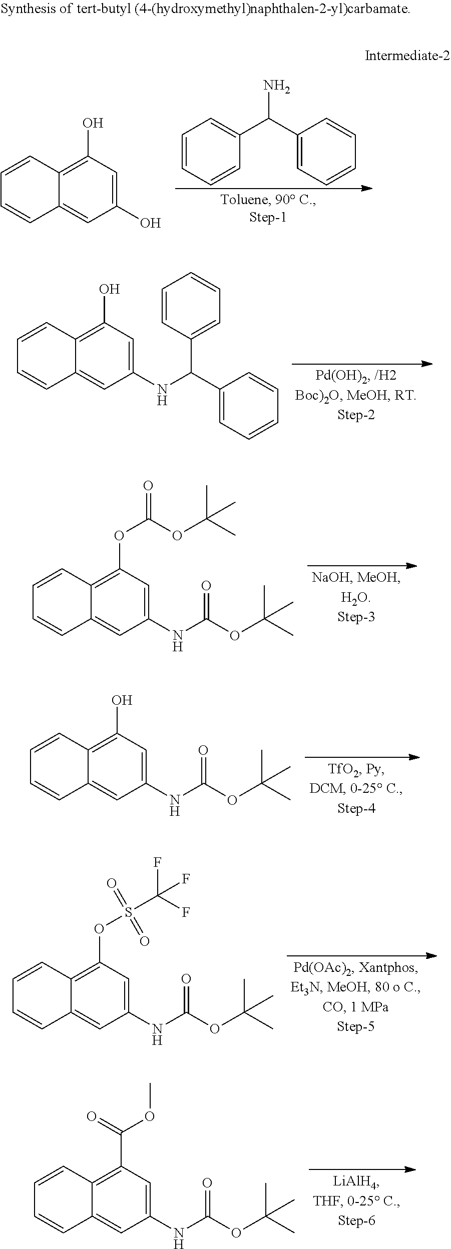

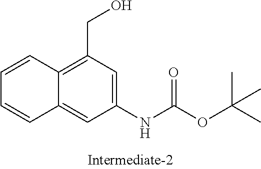 [SMILES: CC(C)(C)OC(=O)Nc1cc(CO)c2ccccc2c1]

### Step-1: Synthesis of 3-(benzhydrylamino)naphthalen-1-ol

To a mixture of naphthalene-1,3-diol (300 g, 1870 mmol) in toluene (2500 mL) was added diphenylmethanamine (500 g, 2730 mmol, 471 mL) in one portion at RT under nitrogen atmosphere. The mixture was stirred at 90° C. for 12 h. The reaction mixture was cooled to RT, filtered, and the filtrate was concentrated to yield the title compound obtained as black-brown oil (600 g, crude yield). This crude product was used as such in the next step without any purifications. LC-MS: 326.2 [M+H]⁺.

### Step-2: Synthesis of tert-butyl (4-((tert-butoxycarbonyl)oxy)naphthalen-2-yl)carbamate

To a solution of 3-(benzhydrylamino)naphthalen-1-ol (423 g, 1290 mmol) in MeOH (1000 mL) were added Pd(OH)₂ (45.8 g, 65.3 mmol, 20% load) and (Boc)₂O (133 g, 610 mmol) under nitrogen atmosphere. The suspension was degassed using vacuum and purged with hydrogen gas several times, stirred under a positive pressure of hydrogen (40 Psi) at RT for 24 h. The reaction mixture was filtered, and the filtrate was concentrated to yield the title compound as brown solid (1000 g. crude yield). LC-MS: 361.1 [M+H]⁺.

### Step-3: Synthesis of tert-butyl (4-hydroxynaphthalen-2-yl)carbamate

To a solution of tert-butyl (4-((tert-butoxycarbonyl)oxy)naphthalen-2-yl)carbamate (500 g, 1390 mmol) in MeOH (2500 mL) was added Aqueous solution of NaOH (5M, 1000 mL) drop-wise at RT and stirred for 2 h. The reaction mixture was concentrated, the residue was dissolved in ethyl acetate, washed with water and then by brine solution, dried over anhydrous magnesium sulfate and filtrated. The filtrate was concentrated to yield the title compound as black oil (660 g, 45.7%). This was used as such in the next step. LC-MS: 260.2 [M+H]⁺.

### Step-4: Synthesis of 3-(tert-butoxycarbonyl)amino)naphthalen-1-yl trifluoromethanesulfonate

A solution of tert-butyl (4-hydroxynaphthalen-2-yl)carbamate (330 g, 636.33 mmol) and pyridine (302 g, 3820 mmol, 308 mL) in DCM (2000 mL) was cooled to 0° C. and to the solution was added Tf₂O (359 g, 1270 mmol, 210 mL) drop-wise over a period of 5 mins under nitrogen atmosphere. After completion of addition, the reaction mixture was stirred at RT for 16 h. The reaction mixture was added water, the organic portion was washed with brine, dried over anhydrous Na₂SO₄ and concentrated. The residue was purified by silica gel MPLC using 30% ethyl acetate in hexane as eluent, the compound obtained was triturated with hexane and stirred for 12 h, filtered and dried to yield the title compound as white solid (350 g, 63.2%).

### Step-5: Synthesis of methyl 3-((tert-butoxycarbonyl)amino)-1-naphthoate

To a solution of 3-((tert-butoxycarbonyl)amino)naphthalen-1-yl trifluoromethanesulfonate (100 g, 255 mmol) in MeOH (2000 mL) was added Xantphos (14.78 g, 25.55 mmol). Pd(OAc)₂ (5.74 g, 25.5 mmol) and Et₃N (77.5 g, 766 mmol, 106 mL) under nitrogen atmosphere. The suspension was degassed using vacuum and purged with carbon monoxide gas several times. The mixture was stirred under a positive pressure of carbon monoxide (1 Mpa) at 80° C. for 16 hours. The reaction mixture was cooled to RT, filtered through Celite and the filtrate was concentrated to yield the title compound as a yellow solid (75 g, 98.1%), ¹H NMR (CDCl₃, 400 MHz): δ 1.48 (s, 9H), 3.91 (s, 3H), 6.68 (s, 1H), 7.38-7.44 (m, 2H), 7.68-7.75 (m, 1H), 8.00 (d, 1H), 8.13 (bs, 1H), 8.62-8.68 (m, 1H).

### Step-6: Synthesis of tert-butyl (4-(hydroxymethyl)naphthalen-2-yl)carbamate

To a solution of methyl 3-((tert-butoxycarbonyl)amino)-1-naphthoate (22.5 g, 74.7 mmol) in THF (100 mL) was added LiAlH₄ (5.67 g, 149 mmol) in one portion at 0° C. under nitrogen atmosphere. The reaction mixture was stirred at RT for 1 h and quenched with water (12 mL) at 0° C. and was stirred at RT for 30 minutes. Solid separated was filtered and the filtrate was concentrated to yield the title compound as yellow solids (20 g, crude yield). ¹H NMR: (CDCl₃, 400 MHz): δ 1.48 (s, 9H), 5.01 (s, 2H), 6.68 (br s, 1H), 7.29-7.41 (m, 3H), 7.64-7.75 (m, 1H), 7.81-7.94 (m, 2H).

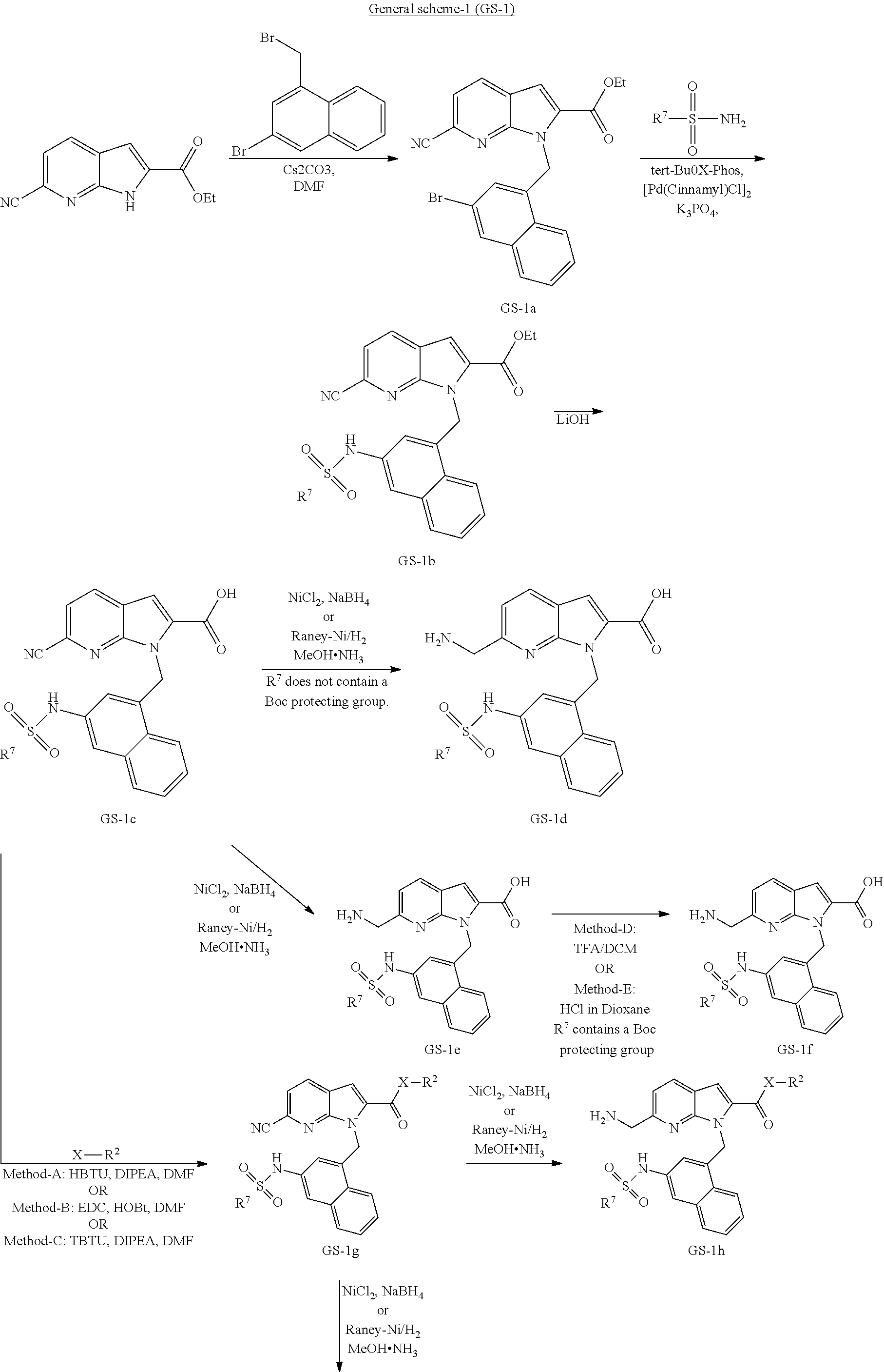

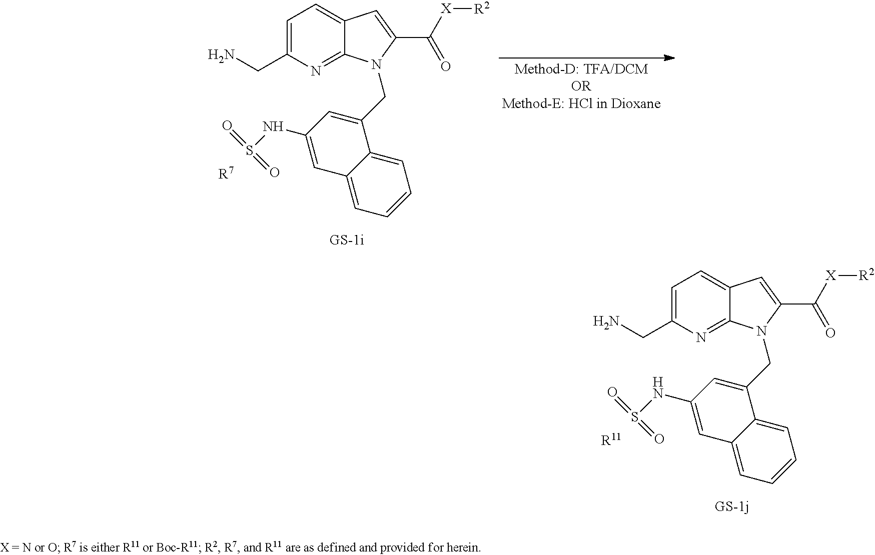

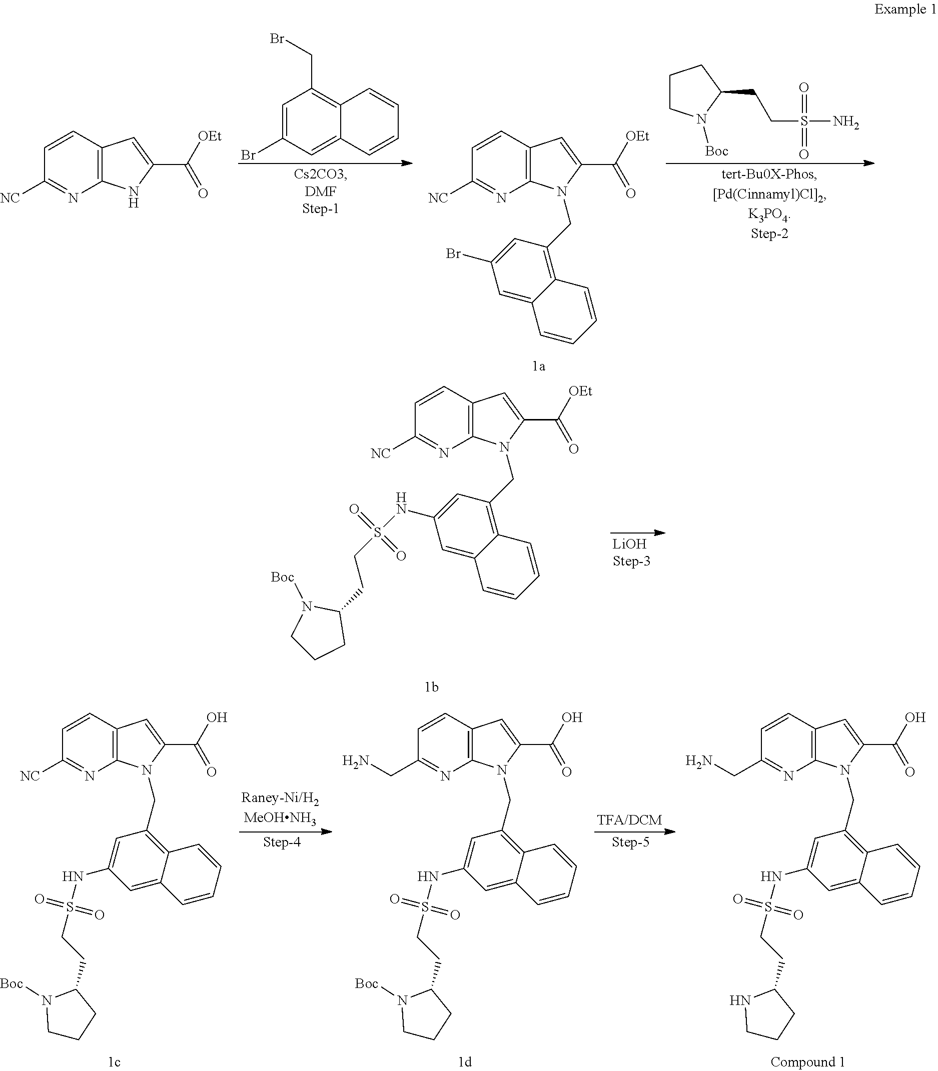

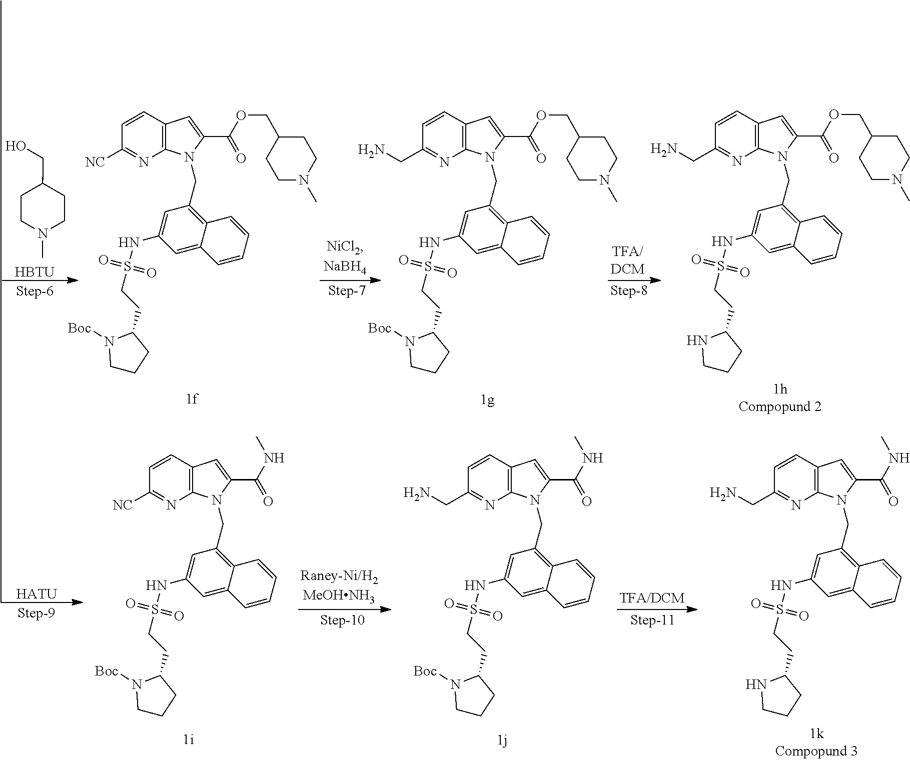

### Step-1: Synthesis of Synthesis of ethyl 1-((3-bromonaphthalen-1-yl)methyl)-6-cyano-1H-pyrrolo[2,3-b]pyridine-2-carboxylate

To a solution of ethyl 6-cyano-1H-pyrrolo[2,3-b]pyridine-2-carboxylate (3.17 g, 14.7 mmol) in DMF (50 mL) was added Cs₂CO₃ (21.33 g, 65.4 mmol) and 3-bromo-1-(bromomethyl)naphthalene (4.9 g, 16.3 mmol) respectively. After addition, the mixture was stirred at RT for 15 h. The reaction mixture was poured into ice-cold water, the precipitate formed was filtered and dried to yield the title compound 1a (6.0 g, 84%). LC-MS: 434.2 [M+H]⁺.

### Step-2: Synthesis of ethyl(S)-1-((3-((2-(1-(tert-butoxycarbonyl)pyrrolidin-2-yl)ethyl)sulfonamido)naphthalen-1-yl)methyl)-6-cyano-1H-pyrrolo[2,3-b]pyridine-2-carboxylate

A solution of ethyl 1-((3-bromonaphthalen-1-yl)methyl)-6-cyano-1H-pyrrolo[2,3-b]pyridine-2-carboxylate (2.5 g, 5.76 mmol, 1.0 eq), tert-butyl(S)-2-(2-sulfamoylethyl)pyrrolidine-1-carboxylate (2.35 g, 8.44 mmol) in 1,4-Dioxane (60 mL) was purged with nitrogen, added K₃PO₄ (3.52 g, 16.6 mmol) followed by Palladium Cinnamyl Chloride dimer (298 mg, 0.575 mmol) and tert butyl-XPhos (0.488 g, 1.15 mmol). After addition, the mixture was stirred at 90° C. for 15 h. The reaction mixture was then filtered through Celite, washed with ethyl acetate and the filtrate was concentrated to get crude. The crude compound was purified by combi flash column chromatography (liquid packing). By eluting with of 40% EtOAc in Hexane. This afforded the pure title compound 1b (2.35 g, 70%.) LC-MS: 631.7 [M+H]⁺.

### Step-3: Synthesis of(S)-1-(3-((2-(1-(tert-butoxycarbonyl)pyrrolidin-2-yl)ethyl)sulfonamido)naphthalen-1-yl)methyl)-6-cyano-1H-pyrrolo[2,3-b]pyridine-2-carboxylic acid

A solution of ethyl(S)-1-((3-((2-(1-(tert-butoxy carbonyl)pyrrolidin-2-yl)ethyl)sulfonamido)naphthalen-1-yl)methyl)-6-cyano-1H-pyrrolo[2,3-b]pyridine-2-carboxylate (2.2 g, 3.48 mmol) in THF (20 mL) and EtOH (10 mL) was cooled to 0° C. This was added LiOH·H₂O (0.439 g, 10.45 mmol) in water (10 mL). Then the reaction mixture was warmed to RT and stirred for 4 h. The reaction mixture was concentrated, and the residue was dissolved in cold water, acidified with 1N HCl (PH˜4.0-5.0). The product precipitated was filtered and dried under vacuum to yield the title compound 1c (2.0 g, 95%). LC-MS: 504.1 [M+H-Boc]+.

### Step-4: Synthesis of(S)-6-(aminomethyl)-1-((3-((2-(1-(tert-butoxycarbonyl)pyrrolidin-2-ylethyl)sulfonamido)naphthalen-1-yl)methyl)-1H-pyrrolo[2,3-b]pyridine-2-carboxylic acid

To a solution of(S)-1-((3-((2-(1-(tert-butoxycarbonyl)pyrrolidin-2-yl)ethyl)sulfonamido)naphthalen-1-yl)methyl)-6-cyano-1H-pyrrolo[2,3-b]pyridine-2-carboxylic acid (0.250 g, 0.414 mmol) in MeOH (10 mL) was added methanolic ammonia (5 mL) and Raney nickel (0.4 g) at RT and the reaction mixture was stirred for 2 h under positive pressure of hydrogen using a bladder. The reaction mixture was filtered through Celite, and the filtrate was concentrated to yield the title compound 1d (0.2 g, 80%). LC-MS: 608.3 [M+H]⁺.

### Step-5: Synthesis of(S)-6-(aminomethyl)-1-((3-((2-(pyrrolidin-2-yl)ethyl)sulfonamido)naphthalen-1-yl)methyl)-1H-pyrrolo[2,3-b]pyridine-2-carboxylic acid (Compound 1)

A solution of(S)-6-(aminomethyl)-1-((3-((2-(1-(tert-butoxycarbonyl)pyrrolidin-2-yl)ethyl)sulfonamido)naphthalen-1-yl)methyl)-1H-pyrrolo[2,3-b]pyridine-2-carboxylic acid (0.2 g, 0.329 mmol) in DCM (5 mL) was cooled to 0° C. and to the solution was added TFA (0.3 mL). The reaction mixture was stirred at RT for 5 h and concentrated to get the crude mass. This was purified by preparative-HPLC to afford pure title compound 1e (Compound 1) (0.025 g, 15.0%). (Preparative HPLC method: Column-LUNA C18 (250 mm×21.2 mm), 5.0μ; Eluent-A 0.1% TFA in water, B—is (1:1) acetonitrile and methanol, gradient—15% B at 0-minute, 25% B at 2 minutes, 55% at 10 minutes). LC-MS: 508.3 [M+H]⁺; ¹HNMR (CD₃OD, 300 MHz): δ 1.44-1.51 (m, 1H), 1.87-2.00 (m, 3H), 2.04-2.13 (m, 2H), 3.03-3.08 (m, 2H), 3.18-3.28 (m, 2H), 3.44-3.52 (m, 1H), 4.299 (s, 2H), 6.19 (s, 1H), 6.48 (s, 2H), 7.23-7.26 (d, 1H), 7.47-7.52 (m, 4H), 7.770-7.79 (m, 1H), 8.16-8.23 (m, 2H).

### Step-6: Synthesis of (1-methylpiperidin-4-yl)methyl(S)-1-((3-((2-(1-(tert-butoxycarbonyl)pyrrolidin-2-yl)ethyl)sulfonamido)naphthalen-1-yl)methyl)-6-cyano-1H-pyrrolo[2,3-b]pyridine-2-carboxylate

A solution of(S)-6-(aminomethyl)-1-((3-((2-(pyrrolidin-2-yl)ethyl)sulfonamido)naphthalen-1-yl)methyl)-1H-pyrrolo[2,3-b]pyridine-2-carboxylic acid (0.1 g, 0.16 mmol), DIPEA (0.088 g, 0.68 mmol) in DMF (1 mL) was cooled to 0° C. and to the solution was added HBTU (0.13 g, 0.33 mmol). After stirring for 10 minutes at the same temperature. (1-methylpiperidin-4-yl)methanol (0.086 g, 0.66 mmol) was added and gradually warmed to RT and stirred for 12 h. The reaction mixture was then poured into ice-cold water, the precipitate formed was filtered, washed and dried. This afforded title compound 1f (0.09 g, 75.8%). LC-MS: 715.50 [M+H]⁺.

### Step-7: (1-methylpiperidin-4-yl)methyl(S)-6-(aminomethyl)-1-((3-((2-(1-(tert-butoxycarbonyl)pyrrolidin-2-yl)ethyl)sulfonamido)naphthalen-1-yl)methyl)-1H-pyrrolo[2,3-b]pyridine-2-carboxylate

A solution of (1-methylpiperidin-4-yl)methyl(S)-1-((3-((2-(1-(tert-butoxycarbonyl)pyrrolidin-2-yl)ethyl)sulfonamido)naphthalen-1-yl)methyl)-6-cyano-1H-pyrrolo[2,3-b]pyridine-2-carboxylate (0.17 g, 0.238 mmol) in methanol (2 mL) was cooled to 0° C., added NiCl₂·6H₂O (0.003 g, 0.01 mmol) and stirred for 5 minutes. Then NaBH₄ (0.027 g, 0.71 mmol) was added in portions by maintaining same temperature. After stirring for 2 h at 0° C., the reaction mixture was quenched with cold water. The precipitate formed was filtered, washed with water and dried to yield the title compound 1g (0.135 g, 78.9%). LC-MS: 719.50 [M+H]⁺.

### Step-8: Synthesis of (1-methylpiperidin-4-yl)methyl (S)-6-(aminomethyl)-1-(3-(2-(pyrrolidin-2-yl)ethyl)sulfonamido)naphthalen-1-yl)methyl)-1H-pyrrolo[2,3-b]pyridine-2-carboxylate. (Compound 2)

A solution of (1-methylpiperidin-4-yl)methyl(S)-6-(aminomethyl)-1-((3-((2-(1-(tert-butoxycarbonyl)pyrrolidin-2-yl)ethyl)sulfonamido)naphthalen-1-yl)methyl)-1H-pyrrolo[2,3-b]pyridine-2-carboxylate (0.15 g, 0.20 mmol) in DCM (3 mL) was cooled to 0° C. and to the solution was added TFA (0.048 g, 1.99 mmol). The reaction mixture was stirred at RT for 2 h and concentrated to get residue. The residue was purified by preparative HPLC to get the pure title compound 1h (Compound 2) (0.022 g, 17.1%). LC-MS: 618.78 [M+H]⁺; ¹HNMR (CD₃OD, 400 MHz): δ 1.43-1.47 (m, 2H), 1.51-1.57 (m, 1H), 1.77-1.78 (d, 2H), 1.91-2.18 (m, 6H), 2.73-2.81 (m, 4H), 3.10-3.14 (t, 2H), 3.32 (s, 3H), 3.39-3.40 (d, 2H), 3.45-3.59 (m, 1H), 4.08-4.09 (d, 2H), 4.34 (s, 2H), 6.24 (s, 1H), 6.50 (s, 2H), 7.29-7.30 (d, 2H), 7.52 (s, 1H), 7.55-7.58 (m, 2H), 7.83-7.85 (m, 1H), 8.21-8.23 (m, 1H), 8.25-8.26 (d, 1H).

### Step-9: Synthesis of tert-butyl(S)-2-(2-(N-(4-((6-cyano-2-(methylcarbamoyl)-1H-pyrrolo[2,3-b]pyridin-1-yl)methyl)naphthalen-2-yl) sulfamoyl)ethyl)pyrrolidine-1-carboxylate

To a solution of(S)-1-((3-((2-(1-(tert-butoxycarbonyl)pyrrolidin-2-yl)ethyl)sulfonamido)naphthalen-1-yl)methyl)-6-cyano-1H-pyrrolo[2,3-b]pyridine-2-carboxylic acid (0.3 g, 0.49 mmol), DIPEA (0.25 mL, 1.49 mmol) in DMF (5 mL) was added HATU (0.28 g, 0.745 mmol) and stirred for 10 minutes. Then methylamine hydrochloride (0.1 g, 0.149 mmol) was added and the mixture was stirred at RT for 4 h. The reaction mixture was added into ice-cold water, the formed precipitate was filtered and washed with water and dried. This was further purified by Combi-flash column chromatography using 0-10% methanol in DCM as eluent to afford the pure title compound 1i (0.21 g, 21.0%). LC-MS: 615.2 [M−H]⁻

### Step-10: Synthesis of ter-butyl(S)-2-(2-(N-(4-((6-(aminomethyl)-2-(methylcarbamoyl)-1H-pyrrolo[2,3-b]pyridin-1-yl)methyl)naphthalen-2-yl) sulfamoyl)ethyl)pyrrolidine-1-carboxylate

A solution of tert-butyl(S)-2-(2-(N-(4-((6-cyano-2-(methylcarbamoyl)-1H-pyrrolo[2,3-b]pyridin-1-yl)methyl)naphthalen-2-yl) sulfamoyl)ethyl)pyrrolidine-1-carboxylate (0.205 g, 0.33 mmol) in methanolic ammonia (2 mL) was added Raney nickel (0.2 g, 3.3 mmol) and stirred under positive pressure of hydrogen for 2 h. Then the reaction mixture was diluted with methanol, filtered through Celite and the filtrate was concentrated to yield the title compound 1j (0.165 g, crude yield). LC-MS: 621.4 [M+H]⁺.

### Step-11: Synthesis of(S)-6-(aminomethyl)-N-methyl-1-(3-(2-(pyrrolidin-2-yl)ethyl)sulfonamido)naphthalen-1-yl)methyl)-1H-pyrrolo[2,3-b]pyridine-2-carboxamide. (Compound 3)

A solution of tert-butyl (S)-2-(2-(N-(4-((6-(aminomethyl)-2-(methylcarbamoyl)-1H-pyrrolo[2,3-b]pyridin-1-yl)methyl)naphthalen-2-yl) sulfamoyl)ethyl)pyrrolidine-1-carboxylate (0.19 g, 0.26 mmol) in DCM (10 mL) was cooled to 0° C. and to the solution was added TFA (4 mL). The reaction mixture was stirred at RT for 4 h and concentrated. The residue was purified by preparative HPLC to afford the pure title compound (Compound 3) (0.022 g, 10%). (Prep HPLC method: Column-ATLANTIS (250 mm×19 mm), flow rate-15 mL/min, mobile phase-A was 0.1% TFA in water. B was acetonitrile). LC-MS: 521.30 [M+H]⁺; ¹HNMR (CD₃OD, 400 MHz): δ 1.44-1.59 (m, 1H), 1.94-2.03 (m, 3H), 2.10-2.12 (t, 1H), 2.77 (s, 3H), 3.10-3.12 (t, 2H), 3.21-3.29 (m, 2H), 3.50-3.58 (d, 2H), 4.31 (s, 2H), 6.43-6.47 (d, 3H), 7.18 (s, 1H), 7.20-7.24 (d, 1H), 7.50-7.54 (m, 3H), 7.78-7.82 (m, 1H), 8.16-8.18 (d, 2H).

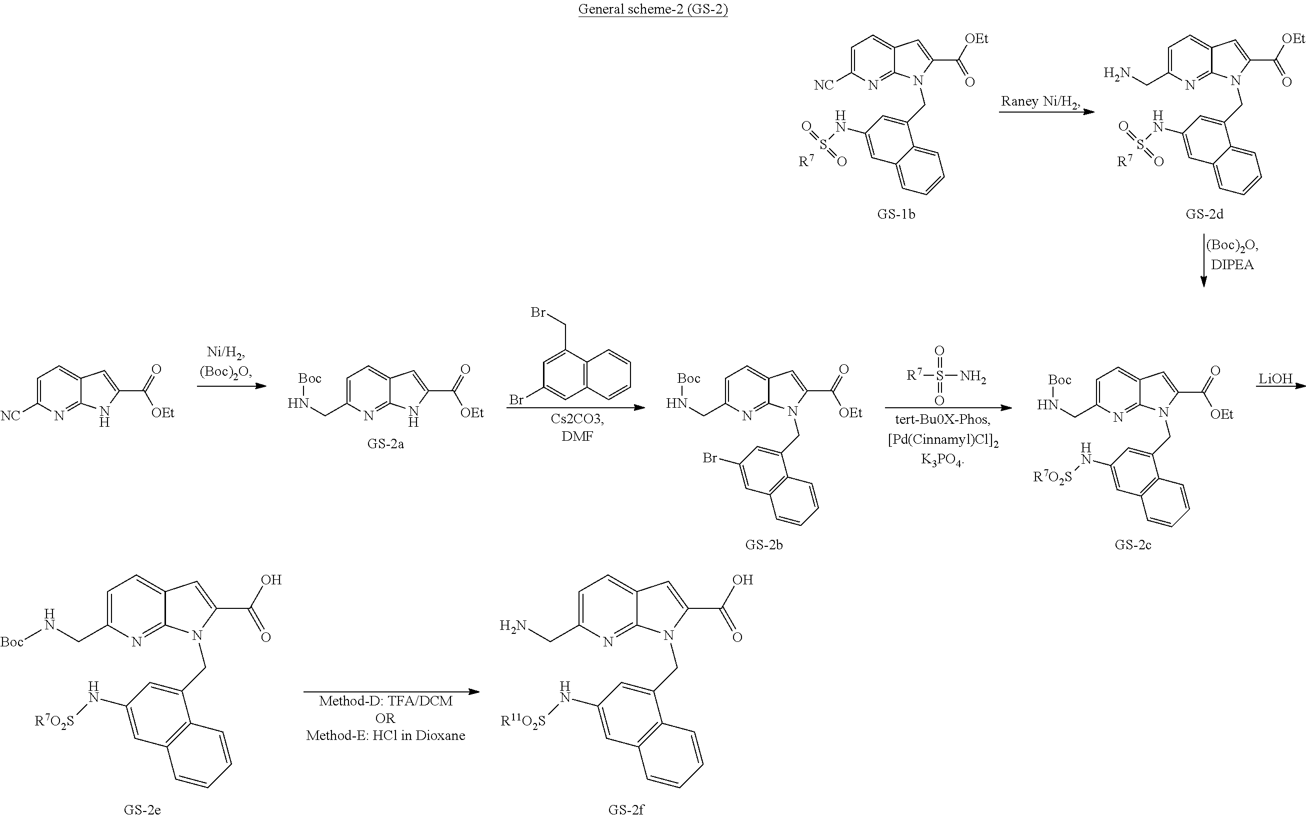

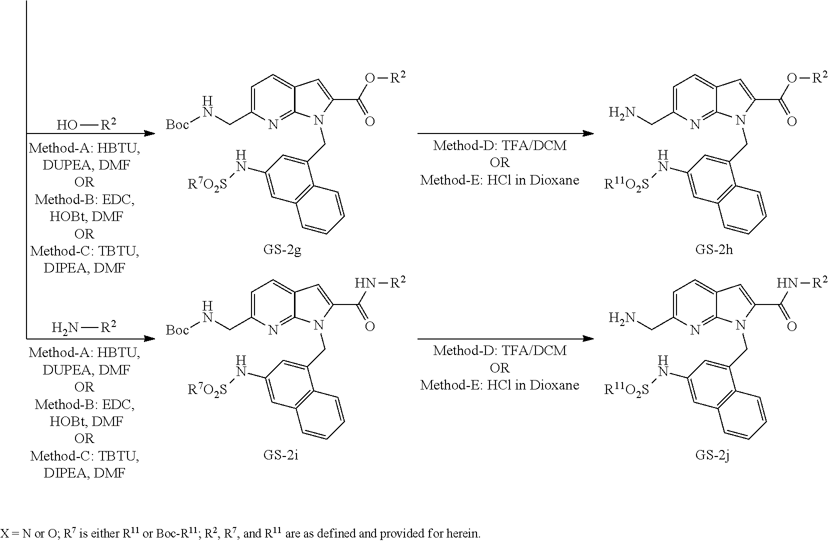

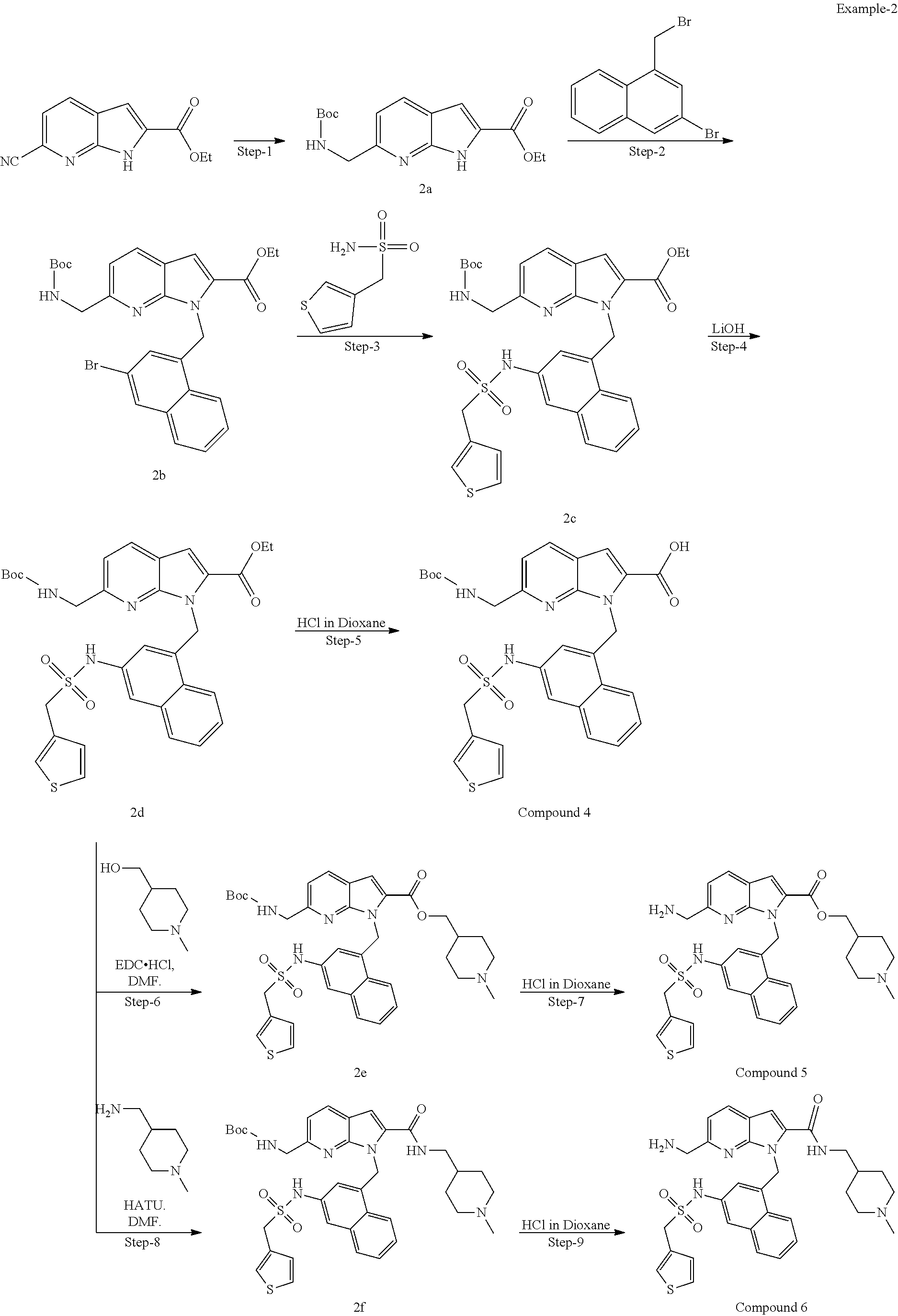

### Step-1: Synthesis of ethyl 6-(((tert-butoxycarbonyl)amino)methyl)-1H-pyrrolo[2,3-b]pyridine-2-carboxylate

A stirred solution of ethyl 6-cyano-1H-pyrrolo[2,3-b]pyridine-2-carboxylate (4 g, 18.5 mmol) in methanol (200 mL) was cooled to 0° C. and to the solution was added Boc anhydride (6.08 g, 27.8 mmol) followed by NiCl₂·6H₂O (0.44 g, 1.85 mmol). NaBH₄ (4.92 g, 130.9 mmol) was added in portions by maintaining the same temperature. The reaction mixture was stirred at RT for 2 h, added diethylenetriamine (1.9 g, 18.5 mmol) and the stirring continued for 1 h. The reaction mixture was concentrated to form a residue that was dissolved in ethyl acetate, washed with sat. NaHCO₃ solution followed by brine solution, dried over Na₂SO₄ and concentrated. The crude mass was purified by Combi-flash column chromatography using 0-30% ethyl acetate in hexane as eluent. This afforded the pure title compound 2a (4 g, 67.3%). LC-MS: 320.1 [M+H]⁺.

### Step-2: Synthesis of ethyl 1-((3-bromonaphthalen-1-yl)methyl)-6-(((tert-butoxycarbonyl)amino)methyl)-1H-pyrrolo[2,3-b]pyridine-2-carboxylate

To a solution of ethyl 6-(((tert-butoxycarbonyl)amino)methyl)-1H-pyrrolo[2,3-b]pyridine-2-carboxylate (4.1 g, 12.8 mmol) in DMF (60 mL) was added Cs₂CO₃ (10.4 g, 32 mmol) followed by 3-bromo-1-(bromomethyl)naphthalene (4.62 g, 15.4 mmol). The reaction mixture was stirred at rt for 18 h. Then the reaction mixture was poured into ice-cold water, the precipitate formed was filtered and dried. The solid obtained was washed with hexane and dried to afford title compound 2b (5.2 g, 75.2%). LC-MS: 540.40 [M+2]⁺.

### Step-3: Synthesis of ethyl 6-(((tert-butoxycarbonyl)amino)methyl)-1-((3-((thiophen-3-ylmethyl)sulfonamido)naphthalen-1-yl)methyl)-1H-pyrrolo[2,3-b]pyridine-2-carboxylate

A mixture of ethyl 1-((3-bromonaphthalen-1-yl)methyl)-6-(((tert-butoxycarbonyl)amino)methyl)-1H-pyrrolo[2,3-b]pyridine-2-carboxylate (2 g, 3.71 mmol), thiophen-3-ylmethanesulfonamide (0.79 g, 4.4 mmol) in dioxane was added K₃PO₄ and purged with N₂. To the reaction mixture were then added tert-butyl-Xphos (0.15 g, 0.17 mmol), palladium (π-cinnamyl) chloride dimer (0.095 g, 0.18 mmol) and the resulting mixture was heated to 90° C. for 12 h. The reaction mixture was filtered through Celite and the collected filtrate was concentrated to get crude mass. This was purified by Combi-flash column chromatography using 0-15% ethyl acetate in hexane as eluent to afford the pure title compound 2c (1.4 g, 59.3%). LC-MS: 632.40 [M−H]⁻.

### Step-4: Synthesis of 6-(((tert-butoxycarbonyl)amino)methyl)-1-((3-((thiophen-3-ylmethyl)sulfonamido)naphthalen-1-yl)methyl)-1H-pyrrolo[2,3-b]pyridine-2-carboxylic acid

A mixture of ethyl 6-(((tert-butoxycarbonyl)amino)methyl)-1-((3-((thiophen-3-ylmethyl)sulfonamido)naphthalen-1-yl)methyl)-1H-pyrrolo[2,3-b]pyridine-2-carboxylate (1.7 g, 2.67 mmol), ethanol (12 mL) and THF (6 mL) was added LiOH hexahydrate (0.67 g, 16.0 mmol) solution in water (12 mL) at RT. The reaction mixture was concentrated and the resulting residue was dissolved in water, extracted with diethyl ether. The aqueous portion was acidified using saturated citric acid extracted with ethyl acetate, dried over Na₂SO₄, concentrated to yield the title compound 2d (1.56 g, 96.05%). LC-MS: 604.30 [M−H]⁻.

### Step-5: Synthesis of 6-(aminomethyl)-1-(3-((thiophen-3-ylmethyl)sulfonamido)naphthalen-1-yl)methyl)-1H-pyrrolo[2,3-b]pyridine-2-carboxylic acid. (Compound 4)

A stirred solution of 6-(((tert-butoxycarbonyl)amino)methyl)-1-((3-((thiophen-3-ylmethyl)sulfonamido)naphthalen-1-yl)methyl)-1H-pyrrolo[2,3-b]pyridine-2-carboxylic acid (0.5 g, 0.82 mmol) in DMF (8 mL) was cooled to 0° C., added HBTU (0.62 g, 1.64 mmol), N,N-DIPEA (0.42 g, 3.29 mmol) and stirred for 15 minutes at the same temperature. Then to the reaction mixture was added (1-ethylpiperidin-4-yl)methanol (0.42 g, 3.29 mmol) and the mixture stirred for 15 h at RT. The reaction mixture was quenched by adding water, precipitated formed was filtered, washed with water and dried. The crude compound was purified by Combi-flash column chromatography using 0-3.5% methanol in DCM as eluent to afford the pure title compound (Compound 4) (0.48 g, 81.1%). LC-MS: 506.95 [M+H]⁺; ¹HNMR (CD₃OD, 400 MHz): δ 4.27 (s, 2H), 4.33 (s, 2H), 6.18 (s, 1H), 6.56 (s, 2H), 6.85-6.87 (d, 1H), 7.04 (s, 1H), 7.28-7.30 (m, 2H), 7.51-7.59 (m, 4H), 7.80-7.83 (m, 1H), 8.23-8.29 (m, 2H).

### Step-6: Synthesis of (1-methylpiperidin-4-yl)methyl 6-(((tert-butoxycarbonyl)amino)methyl)-1-((3-((thiophen-3-ylmethyl)sulfonamido)naphthalen-1-yl)methyl)-1H-pyrrolo[2,3-b]pyridine-2-carboxylate

To a solution of 6-(((tert-butoxy carbonyl)amino)methyl)-1-((3-((thiophen-3-ylmethyl)sulfonamido)naphthalen-1-yl)methyl)-1H-pyrrolo[2,3-b]pyridine-2-carboxylic acid (0.25 g, 0.412 mmol) in DMF (4 mL) was added EDC·HCl (0.192 g, 1.48 mmol), catalytic amount of DMAP and stirred for 10 minutes. Then (1-methylpiperidin-4-yl)methanol (0.21 g, 1.67 mmol) was added and the mixture was stirred at RT for 18 h. The reaction mixture was added into ice-cold water, and the resulting precipitate was filtered and washed with water and dried. This was further purified by Combi-flash column chromatography using 0-4.5% methanol in DCM as eluent to afford the pure title compound 2e (0.15 g, 50.7%). LC-MS: 718.05 [M+H]⁺.

### Step-7: Synthesis of (1-methylpiperidin-4-yl)methyl 6-(aminomethyl)-1-(3-((thiophen-3-ylmethyl)sulfonamido)naphthalen-1-yl)methyl)-1H-pyrrolo[2,3-b]pyridine-2-carboxylate. (Compound 5)

To a stirred solution of tert-butyl (1-methylpiperidin-4-yl)methyl 6-(((tert-butoxycarbonyl)amino)methyl)-1-((3-((thiophen-3-ylmethyl)sulfonamido)naphthalen-1-yl)methyl)-1H-pyrrolo[2,3-b]pyridine-2-carboxylate (0.15 g, 0.209 mmol) in 1,4-dioxane (4 mL) was added 4M HCl in 1,4-dioxane (6 mL) and stirred for 5 h at RT. The reaction mixture was then concentrated, added diethyl ether and filtered to get pure title compound (Compound 5) (0.07 g, 54.2%). LC-MS: 616.2 [M−H]⁻; ¹HNMR (CD₃OD. 400 MHz): δ 1.38-1.47 (m, 2H), 1.75-1.82 (m, 4H), 2.71-2.79 (m, 4H), 3.34-3.40 (d, 2H), 4.10-4.11 (d, 2H), 4.24 (s, 2H), 4.36 (s, 2H), 6.23 (s, 1H), 6.55 (s, 2H), 6.88-6.89 (d, 1H), 7.06-7.07 (d, 1H), 7.29-7.31 (m, 1H), 7.33-7.35 (d, 1H), 7.45 (s, 1H), 7.57-7.62 (m, 3H), 7.83-7.85 (m, 1H), 8.24-8.26 (m, 1H), 8.30-8.32 (d, 1H).

### Step-8: Synthesis of tert-butyl ((2-(((1-methylpiperidin-4-yl)methyl) carbamoyl)-1-((3-((thiophen-3-ylmethyl)sulfonamido)naphthalen-1-yl)methyl)-1H-pyrrolo[2,3-b]pyridin-6-yl)methyl)carbamate

To a solution of 6-(((tert-butoxycarbonyl)amino)methyl)-1-((3-((thiophen-3-ylmethyl)sulfonamido)naphthalen-1-yl)methyl)-1H-pyrrolo[2,3-b]pyridine-2-carboxylic acid (0.3 g, 0.49 mmol), DIPEA (0.192 g, 1.48 mmol) in DMF (4 mL) was added HATU (0.282 g, 0.74 mmol) and stirred for 10 minutes. Then (1-methylpiperidin-4-yl) methanamine (0.095 g, 0.74 mmol) was added and the mixture was stirred at RT for 3 h. The reaction mixture was added into ice-cold water, and the resulting precipitate was filtered and washed with water and dried. This was further purified by Combi-flash column chromatography using 0-10% methanol in DCM as eluent to afford the pure title compound 2f (0.14 g, 39.5%). LC-MS: 717.3 [M+H]⁺.

### Step-9: Synthesis of 6-(aminomethyl)-N-((1-methylpiperidin-4-yl)methyl)-1-((3-((thiophen-3-ylmethyl)sulfonamido)naphthalen-1-yl)methyl)-1H-pyrrolo[2,3-b]pyridine-2-carboxamide. (Compound 6)

To a stirred solution of tert-butyl ((2-(((1-methylpiperidin-4-yl)methyl) carbamoyl)-1-((3-((thiophen-3-ylmethyl)sulfonamido)naphthalen-1-yl)methyl)-1H-pyrrolo[2,3-b]pyridin-6-yl)methyl)carbamate (0.13 g, 0.18 mmol) in 1,4-dioxane (6 mL) was added 4M HCl in 1,4-dioxane (4 mL) and stirred for 12 h at RT. The reaction mixture was then concentrated to get the pure title compound (Compound 6) (0.05 g, 44.7%). LC-MS: 615.2 [M−H]⁻; ¹HNMR (CD₃OD, 400 MHz): δ 1.24-1.33 (m, 3H), 1.66-1.73 (t, 3H), 2.72-2.81 (m, 4H), 3.14-3.16 (d, 2H), 3.30 (s, 2H), 4.38 (s, 2H), 4.48 (s, 2H), 6.45 (s, 1H), 6.53-6.55 (d, 2H), 6.86-6.91 (m, 2H), 7.25-7.27 (d, 1H), 7.32-7.35 (t, 2H), 7.46 (s, 1H), 7.50-7.55 (m, 2H), 7.80-7.82 (d, 1H), 8.15-8.17 (d, 1H), 8.24-8.26 (d, 1H).

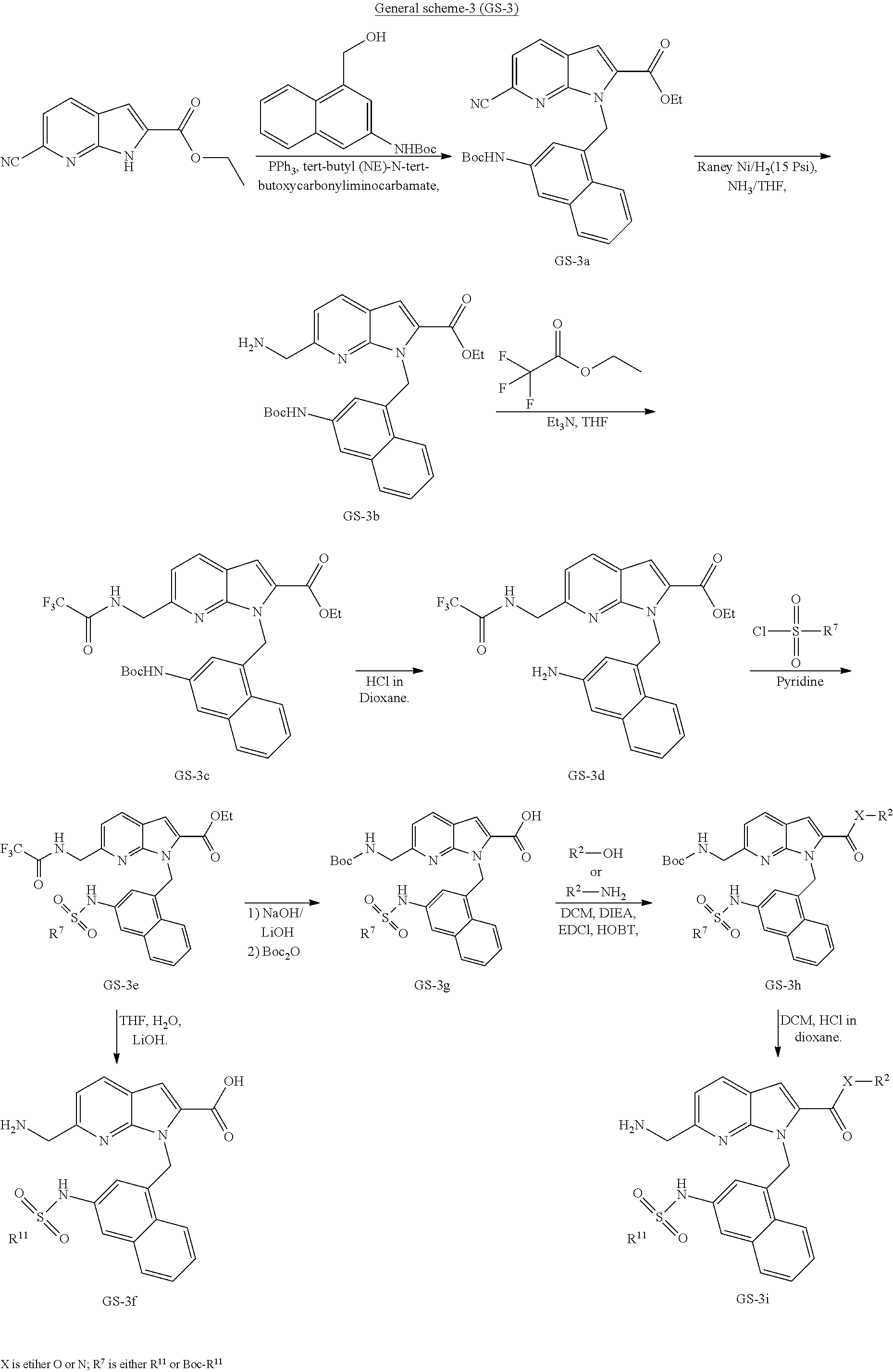

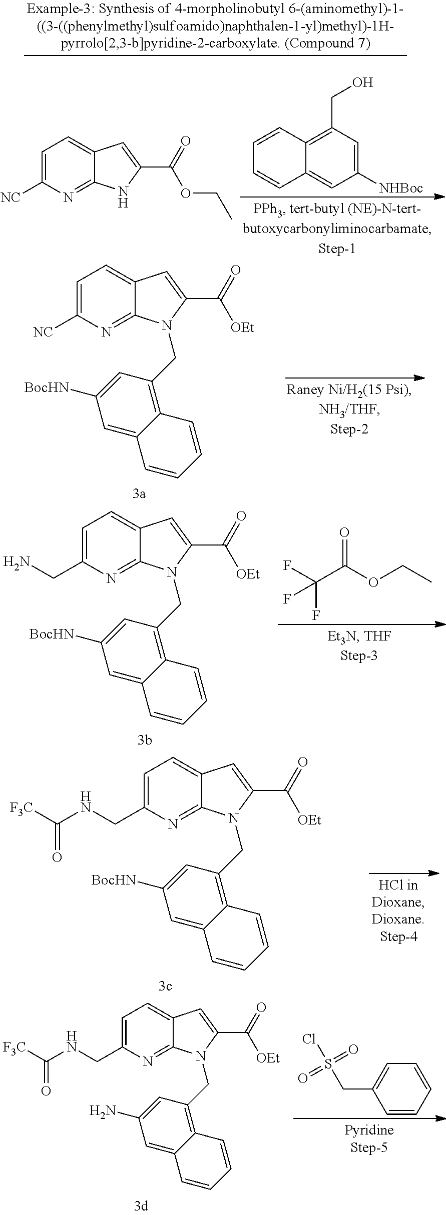

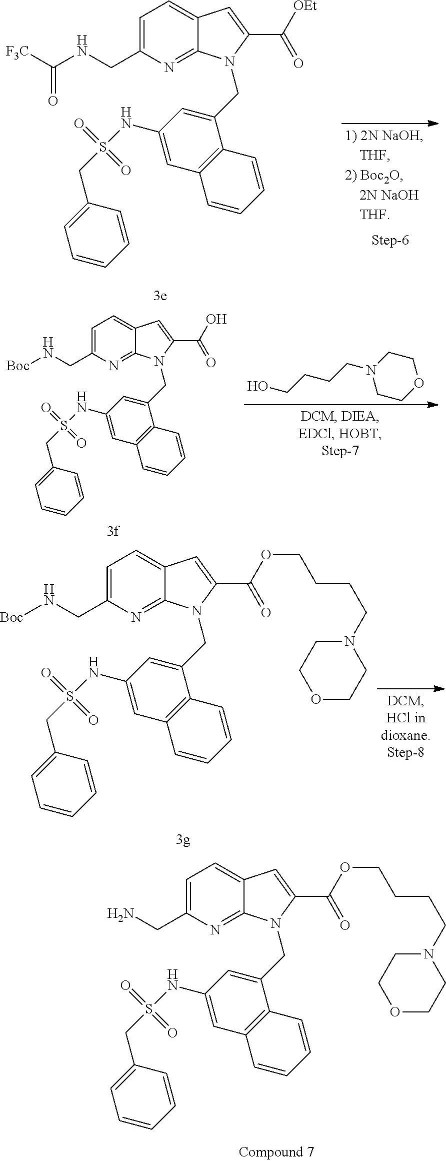 [SMILES: CCOC(=O)c1cc2ccc(CNC(=O)C(F)(F)F)nc2n1Cc1cc(NS(=O)(=O)Cc2ccccc2)cc2ccccc12 ; CC(C)(C)OC(=O)NCc1ccc2cc(C(=O)O)n(Cc3cc(NS(=O)(=O)Cc4ccccc4)cc4ccccc34)c2n1 ; OCCCCN1CCOCC1 ; CC(C)(C)OC(=O)NCc1ccc2cc(C(=O)OCCCCN3CCOCC3)n(Cc3cc(NS(=O)(=O)Cc4ccccc4)cc4ccccc34)c2n1 ; NCc1ccc2cc(C(=O)OCCCCN3CCOCC3)n(Cc3cc(NS(=O)(=O)Cc4ccccc4)cc4ccccc34)c2n1]

### Step-1: Synthesis of ethyl 1-((3-((tert-butoxycarbonyl)amino)naphthalen-1-yl)methyl)-6-cyano-1H-pyrrolo[2,3-b]pyridine-2-carboxylate

To a mixture of tert-butyl (4-(hydroxymethyl)naphthalen-2-yl)carbamate (30.0 g, 109 mmol) and ethyl 6-cyano-1H-pyrrolo[2,3-b]pyridine-2-carboxylate (23.6 g, 109 mmol) in THF (1000 mL) was added PPh₃ (43.2 g, 164 mmol) and tert-butyl (NE)-N-tert-butoxycarbonyliminocarbamate (37.9 g, 164 mmol,) in one portion at RT under nitrogen atmosphere. The mixture was stirred at RT for 2 h. To the mixture was added water and the mixture was extracted with ethyl acetate and concentrated. The residue was purified by silica gel MPLC using 20% ethyl acetate in hexane to afford the pure title compound 3a as yellow solids (41.0 g, 75.4%), ¹H NMR (DMSO-d₆, 400 MHz): δ 1.16-1.12 (m, 3H), 1.40 (s, 9H), 4.21 (m, 2H), 6.19 (s, 1H), 6.31 (s, 2H), 7.58-7.48 (m, 2H), 7.65 (s, 1H), 7.91-7.80 (m, 2H), 8.22-8.07 (m, 2H), 8.56 (d, 1H), 9.16 (s, 1H).

### Step-2: Synthesis of ethyl 6-(aminomethyl)-1-((3-((tert-butoxycarbonyl)amino)naphthalen-1-yl)methyl)-1H-pyrrolo[2,3-b]pyridine-2-carboxylate

Ammonia in THF (200 mL) was added to ethyl 1-((3-((tert-butoxycarbonyl)amino)naphthalen-1-yl)methyl)-6-cyano-1H-pyrrolo[2,3-b]pyridine-2-carboxylate (41.0 g, 87.1 mmol) in THF (500 mL) followed by Raney-Nickel (5 g, 58.3 mmol) was added under nitrogen atmosphere. The mixture was stirred under a positive pressure of hydrogen (15 Psi) at RT for 16 h. The reaction mixture was then filtered and the filtrate was concentrated to yield the title compound 3b as a yellow oil (45 g, crude yield). This was used as such in the next step. LC-MS: 475.3 [M+H]⁺.

### Step-3: Synthesis of ethyl 1-((3-(tert-butoxycarbonyl)amino)naphthalen-1-yl)methyl)-6-((2,2,2-trifluoroacetamido)methyl)-1H-pyrrolo[2,3-b]pyridine-2-carboxylate

To a mixture of ethyl 6-(aminomethyl)-1-((3-((tert-butoxycarbonyl)amino)naphthalen-1-yl)methyl)-1H-pyrrolo[2,3-b]pyridine-2-carboxylate (41.0 g, 86.4 mmol) and Et₃N (52.4 g, 518 mmol, 72.1 mL) in THF (1000 mL) was added ethyl 2,2,2-trifluoroacetate (36.8 g, 259 mmol, 35.7 mL) in one portion at RT under nitrogen atmosphere. The mixture was stirred at RT for 16 h. The reaction mixture was concentrated, and the residue was purified by silica gel MPLC using 20% THF in hexane to afford the pure title compound 3c as yellow solid (43.5 g, 79.4%). LC-MS: 571.3 [M+H]⁺.

### Step-4: Synthesis of ethyl 1-((3-aminonaphthalen-1-yl)methyl)-6-((2,2,2-trifluoroacetamido)methyl)-1H-pyrrolo[2,3-b]pyridine-2-carboxylate

To a mixture of ethyl 1-((3-((tert-butoxycarbonyl)amino)naphthalen-1-yl)methyl)-6-((2,2,2-trifluoroacetamido)methyl)-1H-pyrrolo[2,3-b]pyridine-2-carboxylate (43.5 g, 76.2 mmol) in dioxane (250 mL) was added 4M HCl in dioxane (250 mL) in one portion at RT under nitrogen atmosphere. The mixture was stirred at RT for 16 h. The reaction mixture was concentrated to get title compound 3d as yellow solid. (37.1 g, 93.1%) was obtained as yellow solid. LC-MS: 471.2 [M+H]⁺.

### Step-5: Synthesis of ethyl 1-((3-((phenylmethyl)sulfonamido)naphthalen-1-yl)methyl)-6-((2,2,2-trifluoroacetamido)methyl)-1H-pyrrolo[2,3-b]pyridine-2-carboxylate

To a mixture of ethyl 1-((3-aminonaphthalen-1-yl)methyl)-6-((2,2,2-trifluoroacetamido)methyl)-1H-pyrrolo[2,3-b]pyridine-2-carboxylate (6 g, 12.7 mmol) in pyridine (120 mL) was added phenylmethanesulfonyl chloride (4.86 g, 25.51 mmol) in one portion at RT under nitrogen atmosphere. After stirring for 2 h a RT, the mixture was poured into water (300 mL) and stirred for 5 minutes, extracted with ethyl acetate. The organic portion was washed with brine, dried over anhydrous MgSO₄ and concentrated. The residue was purified by silica gel MPLC using 50% THF in hexane to get pure title compound 3e as a yellow solid (5.5 g, 65.6%). LC-MS: 625.2 [M+H]⁺.

### Step-6: Synthesis of 6-(((tert-butoxycarbonyl)amino methyl)-1-(3-((phenylmethyl)sulfonamido)naphthalen-1-yl)methyl)-1H-pyrrolo[2,3-b]pyridine-2-carboxylic acid

To a mixture of ethyl 1-((3-((phenylmethyl)sulfonamido)naphthalen-1-yl)methyl)-6-((2,2,2-trifluoroacetamido)methyl)-1H-pyrrolo[2,3-b]pyridine-2-carboxylate (5.5 g, 8.81 mmol) in THF (50 mL) was added Aqueous 2M NaOH (62.5 mL) in one portion at RT. The mixture was stirred RT for 16 h. This was added (Boc)₂₀ (5.77 g, 26.4 mmol, 6.07 mL) and stirred at RT for 2 h. The reaction mixture was concentrated, and the residue was neutralized with 1M hydrochloric acid to pH 5. Solid separated was extracted with ethyl acetate-THF (1:1). The combined organic portion was dried over MgSO₄, and concentrated. The residue was purified by silica gel MPLC using 10% MeOH in DCM as eluent. This afforded the pure title compound 3f as yellow solid (4.20 g, 79.4%). LC-MS: 601.2 [M+H]⁺.

### Step-7: Synthesis of 4-morpholinobutyl 6-(((tert-butoxycarbonyl)amino)methyl)-1-((3-((phenylmethyl)sulfonamido)naphthalen-1-yl)methyl)-1H-pyrrolo[2,3-b]pyridine-2-carboxylate

To a solution of 6-(((tert-butoxycarbonyl)amino)methyl)-1-((3-(phenylmethylsulfonamido) naphthalen-1-yl)methyl)-1H-pyrrolo[2,3-b]pyridine-2-carboxylic acid (0.15 g, 0.250 mmol) and 4-morpholinobutan-1-ol (0.059 g, 0.375 mmol) in DCM (4 mL) was added DIEA (0.23 mL, 1.25 mmol). EDCI (0.057 g, 0.3 mmol) and HOBT (0.040 g, 0.3 mmol) at RT. The resulting mixture was stirred at 50° C. for 16 h. The reaction mixture was concentrated to yield the title compound 3g. The crude product was used into the next step without further purification.

### Step 8: 4-morpholinobutyl 6-(aminomethyl)-1-((3-((phenylmethyl)sulfonamido)naphthalen-1-yl)methyl)-1H-pyrrolo[2,3-b]pyridine-2-carboxylate. (Compound 7)

To a solution of 4-morpholinobutyl 6-(((tert-butoxy carbonyl)amino)methyl)-1-((3-(phenylmethylsulfonamido)naphthalen-1-yl)methyl)-1H-pyrrolo[2,3-b]pyridine-2-carboxylate in DCM (2 mL) was added 4M HCl in dioxane (2 mL). The reaction mixture was stirred at 30° C. for 2 h. The reaction mixture was concentrated to get the residue. The residue was purified by preparative HPLC to obtain the pure title compound (Compound 7) (0.048 g). LC-MS: 642.3 [M+H]⁺.

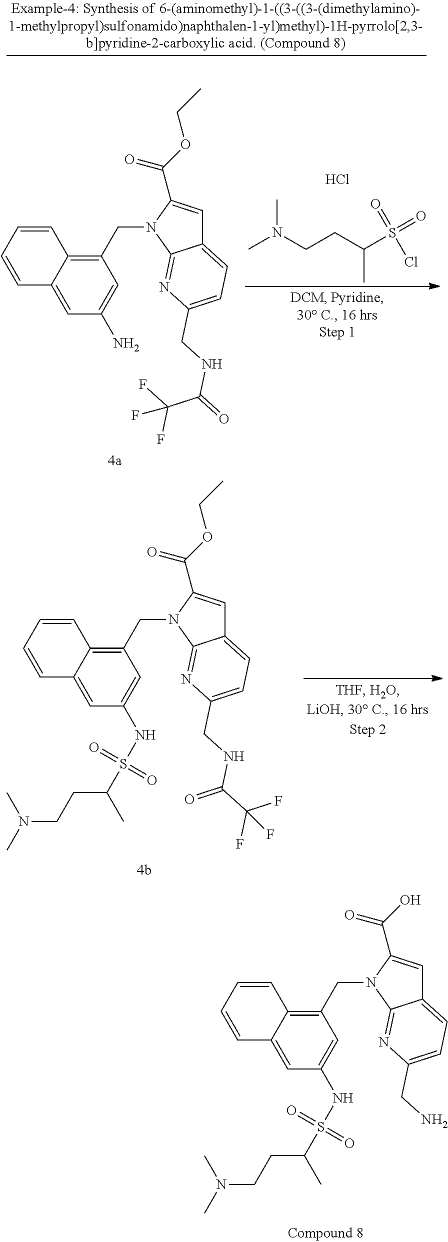

### Step-1: Synthesis of ethyl 1-((3-((3-(dimethylamino)-1-methylpropyl)sulfonamido)naphthalen-1-yl)methyl)-6-((2,2,2-trifluoroacetamido)methyl)-1H-pyrrolo[2,3-b]pyridine-2-carboxylate

To a solution of ethyl 1-((3-aminonaphthalen-1-yl)methyl)-6-((2,2,2-trifluoroacetamido)methyl)-1H-pyrrolo[2,3-b]pyridine-2-carboxylate (4a) (0.1 g, 0.17 mmol) and 4-(dimethylamino) butane-2-sulfonyl chloride hydrochloride (0.60 g, 0.258 mmol) in DCM (4 mL) was added pyridine (0.3 mL, 0.102 mmol) at 0° C. The resulting mixture was stirred at 30° C. for 16 h. The reaction mixture was concentrated get pure title compound 4b.

### Step-2: Synthesis of 6-(aminomethyl)-1-((3-((3-(dimethylamino)-1-methylpropyl)sulfonamido)naphthalen-1-yl)methyl)-1H-pyrrolo[2,3-b]pyridine-2-carboxylic acid. (Compound 8)

To a solution of ethyl 1-((3-(3-(dimethylamino)-1-methylpropylsulfonamido) naphthalen-1-yl)methyl)-6-((2, 2, 2-trifluoroacetamido)methyl)-1H-pyrrolo[2,3-b] pyridine-2-carboxylate (0.1 g, 0.158 mmol) in THF (4 mL), water (1 mL) was added LiOH (0.15 g, 0.632 mmol). The reaction mixture was stirred at 30° C. for 16 h. Reaction mixture was concentrated and purified by preparative HPLC to get pure title compound (Compound 8) (0.25 g). LC-MS: 510.2 [M+H]⁺.

The following compounds listed in Table 1 were prepared according to GS-1 or GS-2 or GS-3 by following the similar procedures as described above for Example-1 or Example-2 or Example-3 or Example-4 using appropriate reagents with suitable modifications known to the one skilled in the art.

TABLE 1

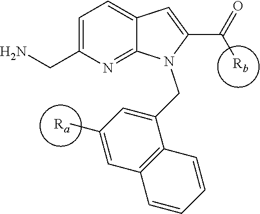

| Cmpd | | | | |
|---|---|---|---|---|
| # | Rₐ | R_{b} | Scheme | Spectral data |
| 9 | 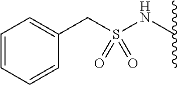 [SMILES: *NS(=O)(=O)Cc1ccccc1] | 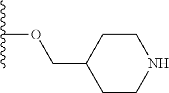 [SMILES: *OCC1CCNCC1] | GS-2 | LC-MS: 598.40[M + H]⁺; ¹HNMR (DMSO-d₆, 300 MHz): δ 1.28-1.33 (m, 2H), 1.51-1.55 (m, 2H), 1.60-1.75 (m, 1H), 2.50-2.65 (m, 2H), 3.06-3.10 (m, 2H), 3.95-3.97 (d, 2H), 4.23-4.30 (m, 4H), 6.18 (s, 1H), 6.44 (s, 2H), 7.04-7.07 |
| | | | | (d, 2H), 7.21-7.30(m, 3H), 7.37-7.40 (d, |
| | | | | 1H), 7.50 (s, 1H), 7.56-7.63 (m, 2H), |
| | | | | 7.86-7.89 (m, 1H), 8.22-8.25 (m, 1H), |
| | | | | 8.32-8.38 (m, 3H), 8.60-8.70 (m, 1H), |
| | | | | 8.80-8.85 (m, 1H),9.79 (s, 1H). |
| 10 | 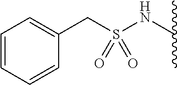 [SMILES: *NS(=O)(=O)Cc1ccccc1] | 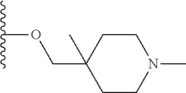 [SMILES: *OCC1(C)CCN(C)CC1] | GS-2 | LC-MS: 626.5 [M + H]⁺; ¹HNMR (CD₃OD, 400 MHz): δ 1.45-1.48 (d, 2H), 1.69-1.72 (d, 2H), 2.01(s, 1H), 2.68 (s, 1H), 2.80-2.81 (d, 2H), 2.92-2.98 (d, 1H), 3.01-3.04 (t, 2H), 3.10 (d, 2H), 3.98 (s,2H), 4.15 (s, 2H), 4.22 (s, 1H), 4.34 (s, |
| | | | | 2H), 6.27 (s, 1H), 6.54 (s, 2H), 7.07 (d, |
| | | | | 2H), 7.21-7.26 (m, 2H), 7.30-7.32 (d, |
| | | | | 2H), 7.37 (s, 1H), 7.55-7.57 (m, 2H), |
| | | | | 7.62 (s, 1H), 7.77-7.79 (m, 1H), 8.23 (s, |
| | | | | 1H), 8.29-8.30 (d, 1H). |
| 11 | 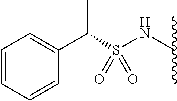 [SMILES: *NS(=O)(=O)[C@@H](C)c1ccccc1] | 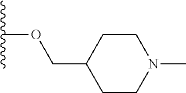 [SMILES: *OCC1CCN(C)CC1] | GS-2 | LC-MS: 626.4 [M + H]⁺; ¹HNMR (CD₃OD, 400 MHz): δ 1.36-1.46 (m, 2H), 1.55-1.56 (d, 3H), 1.71-1.80 (m, 3H), 2.68-2.76 (m, 5H), 3.33-3.36 (d, 2H), 4.08-4.12 (m, 3H), 4.34 (s, 2H), 6.29- 6.30 (d, 1H), 6.47-6.58 (q, 2H), 7.09-7.12 (d, 2H), 7.18-7.27 (m, 4H), 7.31-7.33 (d, |
| | | | | 1H), 7.53-7.61 (m, 3H), 7.75-7.77 (m, |
| | | | | 1H), 8.22-8.24 (m, 1H), 8.29-8.31 (d, |
| | | | | 1H). |
| 12 | 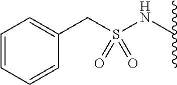 [SMILES: *NS(=O)(=O)Cc1ccccc1] | 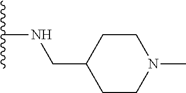 [SMILES: *NCC1CCN(C)CC1] | GS-2 | LC-MS: 611.1 [M + H]⁺; ¹HNMR (CD₃OD, 400 MHz): δ 1.23-1.29 (m, 2H), 1.65-1.72 (m, 2H), 2.71-2.73 (m, 1H), 2.77 (s, 3H), 2.75-2.79 (m, 1H), 2.97- 2.99 (m, 1H), 3.12.-3.14 (d, 2H), 3.34 (s, 2H), 4.26 (s, 2H), 4.38 (s, 2H), 6.49-6.54 |
| | | | | (m, 3H), 7.10-7.12 (d, 2H), 7.21-7.32 (m, |
| | | | | 5H), 7.32 (s, 1H), 7.47-7.55 (m, 2H), |
| | | | | 7.78-7.79 (d, 1H), 8.14-8.16 (d, 1H), |
| | | | | 8.23-8.25 (d, 1H). |
| 13 | 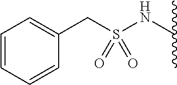 [SMILES: *NS(=O)(=O)Cc1ccccc1] | 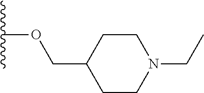 [SMILES: *OCC1CCN(CC)CC1] | GS-3 | LC-MS: 626 [M + H]⁺. |
| 14 | 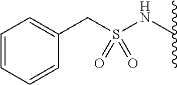 [SMILES: *NS(=O)(=O)Cc1ccccc1] | 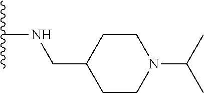 [SMILES: *NCC1CCN(C(C)C)CC1] | GS-3 | LC-MS: 639 [M + H]⁺. |
| 15 | 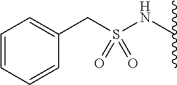 [SMILES: *NS(=O)(=O)Cc1ccccc1] | 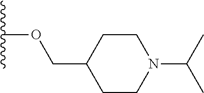 [SMILES: *OCC1CCN(C(C)C)CC1] | GS-3 | LC-MS: 640 [M + H]⁺. |
| 16 | 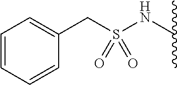 [SMILES: *NS(=O)(=O)Cc1ccccc1] | 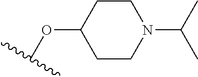 [SMILES: *OC1CCN(C(C)C)CC1] | GS-3 | LC-MS: 626 [M + H]⁺. |
| 17 | 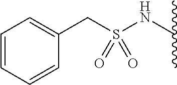 [SMILES: *NS(=O)(=O)Cc1ccccc1] | 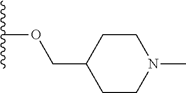 [SMILES: *OCC1CCN(C)CC1] | GS-2 | LC-MS: 612.4 [M + H]⁺; ¹HNMR (CD₃OD, 400 MHz): δ 1.29-1.40 (m, 2H), 1.70-1.95 (m, 2H), 2.68-2.75 (m, 2H), 2.78 (s, 3H), 3.30-3.45 (m, 3H), 4.10- 4.11 (d, 2H), 4.19 (s, 2H),4.36 (s, 2H), 6.29 (s, 1H), 6.56 (s, 2H), 7.08-7.10 (d, |
| | | | | 2H), 7.22-7.34 (m, 4H), 7.40 (s, 1H), |
| | | | | 7.58-7.61 (m, 3H), 7.81-7.84 (m, 1H), |
| | | | | 8.23-8.26 (m, 1H), 8.30-8.33 (d, 1H). |
| 18 | 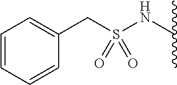 [SMILES: *NS(=O)(=O)Cc1ccccc1] | 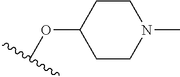 [SMILES: *OC1CCN(C)CC1] | GS-2 | LC-MS: 597.7 [M + H]⁺; ¹HNMR (CD₃OD, 400 MHz): δ 1.94-2.00 (m, 3H), 2.12, 2.54 (s, 3H), 2.73-2.79 (m, 2H), 3.13-3.16 (d, 2H), 4.15-4.15 (bs, 2H), 4.34 (s, 2H), 6.26 (s, 1H), 6.31 (s, 1H), 6.54-6.55 (d, 2H), 7.06-7.09 (m, |
| | | | | 2H), 7.21-7.28 (m, 3H), 7.30-7.34 (m, |
| | | | | 1H), 7.38-7.39 (d, 1H), 7.53-7.62 (m, |
| | | | | 3H), 7.76 (s, 1H), 7.78-7.83 (m, 1H), |
| | | | | 8.22-8.20 (m, 1H), 8.30-8.32 (d, 1H). |
| 19 | 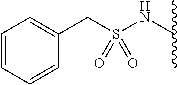 [SMILES: *NS(=O)(=O)Cc1ccccc1] | 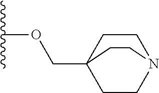 [SMILES: *OCC12CCN(CC1)CC2] | GS-2 | LC-MS: 624.4 [M + H]⁺; ¹HNMR (CD₃OD, 400 MHz): δ 1.54-1.58 (t, 6H), 3.04-3.08 (t, 6H), 4.07 (s, 2H), 4.20 (s, 2H), 4.36 (s, 2H), 6.26 (s, 1H), 6.55 (s, 2H), 7.09-7.10 (d, 2H), 7.21-7.25 (m, 2H), 7.27-7.29 (d, 1H), 7.33-7.35 (d, |
| | | | | 1H), 7.39 (s, 1H), 7.59-7.64 (m, 2H), |
| | | | | 7.66 (s, 1H), 7.82-7.84 (m, 1H), 8.25- |
| | | | | 8.27 (d, 1H), 8.31-8.33 (d, 1H). |
| 20 | 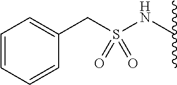 [SMILES: *NS(=O)(=O)Cc1ccccc1] | 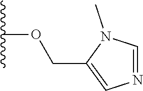 [SMILES: *OCc1cncn1C] | GS-3 | LC-MS: 595 [M + H]⁺ |
| 21 | 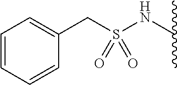 [SMILES: *NS(=O)(=O)Cc1ccccc1] | 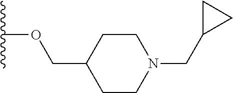 [SMILES: *OCC1CCN(CC2CC2)CC1] | GS-3 | LC-MS: 652 [M + H]⁺. |
| 22 | 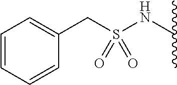 [SMILES: *NS(=O)(=O)Cc1ccccc1] | 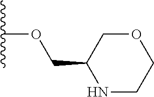 [SMILES: *OC[C@H]1COCCN1] | GS-3 | LC-MS: 600 [M + H]⁺. |
| 23 | 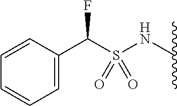 [SMILES: *NS(=O)(=O)[C@H](F)c1ccccc1] | 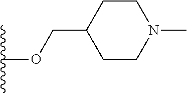 [SMILES: *OCC1CCN(C)CC1] | GS-2 | LC-MS: 630.4 [M + H]⁺; ¹HNMR (CD₃OD, 400 MHz): δ 1.36-1.39 (m, 2H), 1.67-1.74 (m, 3H), 2.61-2.67 (m, 2H), 2.73 (s, 3H), 3.32-3.33 (s, 2H), 4.05-4.07 (d, 2H), 4.33 (s, 2H), 6.00-6.11 (s, 1H), 6.32 (s, 2H), 6.53 (s, 1H), 7.29-7.31 (d, 1H), 7.33-7.46 (m, 6H), 7.55-7.62 (m, |
| | | | | 3H), 7.81-7.83 (d, 1H), 8.23-8.25 (d, |
| | | | | 1H), 8.28-8.30 (d, 1H). |
| 24 | 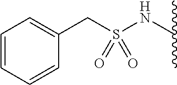 [SMILES: *NS(=O)(=O)Cc1ccccc1] | 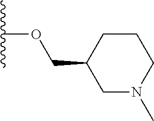 [SMILES: *OC[C@H]1CCCN(C)C1] | GS-2 | LC-MS: 610.2 [M + H]⁺; ¹HNMR (CD₃OD, 400 MHz): δ 1.05-1.14 (m, 1H), 1.58-1.70 (m, 2H), 1.76-1.84 (m, 1H), 2.04-2.18 (m, 1H), 2.42-2.55 (m, 2H), 2.65 (s, 3H), 3.29-3.32 (m, 2H), 4.13- 4.19 (m, 4H), 4.33 (s, 2H), 6.28 (s, 1H), 6.55 (s, 2H), 7.05-7.06 (d, 2H), 7.20-7.27 (m, 3H), 7.31-7.33 (d, 1H), 7.37 (d, 1H), |
| | | | | 7.56-7.60 (m, 2H), 7.66 (s, 1H), 7.80- |
| | | | | 7.84 (m, 1H), 8.24-8.26 (d, 1H), 8.29- |
| | | | | 8.31 (d, 1H). |
| 25 | 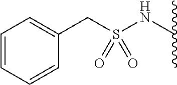 [SMILES: *NS(=O)(=O)Cc1ccccc1] | 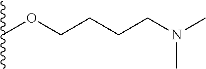 [SMILES: *OCCCCN(C)C] | GS-2 | LC-MS: 600.4 [M + H]⁺; ¹HNMR (CD₃OD, 400 MHz): δ 1.61-1.63 (m, 4H), 2.70 (s, 6H), 2.92-2.94 (t, 2H), 4.15 (s, 2H), 4.22-4.25 (t, 2H), 4.33 (s, 2H), 6.30 (s, 1H), 6.55 (s, 2H), 7.06-7.08 (d, 2H), 7.23-7.32 (m, 4H), 7.37(s, 1H), 7.55-7.59 |
| | | | | (m, 3H), 7.75-7.85 (m, 1H), 8.20-8.31 |
| | | | | (m, 2H). |
| 26 | 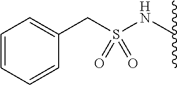 [SMILES: *NS(=O)(=O)Cc1ccccc1] | 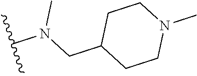 [SMILES: *N(C)CC1CCN(C)CC1] | GS-2 | LC-MS: 625.3 [M + H]⁺; ¹HNMR (CD₃OD, 400 MHz): δ 1.46-1.64 (m, 4H), 2.96 (s, 3H), 3.08 (m, 6H), 3.19-3.21 (d, 2H), 3.30-3.33 (m, 2H), 4.26 (s, 2H), 4.38 (s, 2H), 6.51 (s, 3H), 7.10-7.12 (d, 2H), 7.21-7.32 (m, 6H), 7.32 (s, 1H), |
| | | | | 7.45-7.54 (m, 2H), 7.76-7.79 (d, 1H), |
| | | | | 8.12-8.14 (d, 1H), 8.22-8.25 (d, 1H). |
| 27 | 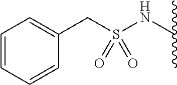 [SMILES: *NS(=O)(=O)Cc1ccccc1] | 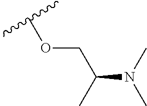 [SMILES: *OC[C@H](C)N(C)C] | GS-3 | LC-MS: 586 [M + H]⁺. |
| 28 | 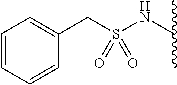 [SMILES: *NS(=O)(=O)Cc1ccccc1] | 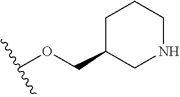 [SMILES: *OC[C@H]1CCCNC1] | GS-2 | LC-MS: 598.2 [M + H]⁺; ¹HNMR (DMSO-d₆, 400 MHz): δ 1.10-1.20 (m, 1H), 1.52-1.59 (m, 3H), 1.90-2.10 (m, 2H), 2.55-2.65 (m, 2H), 3.10-3.22 (m, 2H), 4.04-4.05 (d, 2H), 4.26 (s, 2H), 4.32 (s, 2H), 6.20 (s, 1H), 6.45 (s, 2H), 7.06- 7.08 (m, 2H), 7.23-7.31 (m, 3H), 7.40- |
| | | | | 7.42 (m, 1H), 7.52 (s, 1H), 7.59-7.60- |
| | | | | 7.66 (m, 2H), 7.67 (s, 1H), 7.88-7.90 (m, |
| | | | | 1H), 8.23-8.25 (m, 1H), 8.35-8.38 (m, |
| | | | | 2H), 8.80-8.90 (m, 1H), 9.05-9.15 (m, |
| | | | | 1H). |
| 29 | 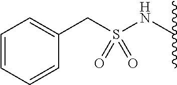 [SMILES: *NS(=O)(=O)Cc1ccccc1] | 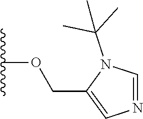 [SMILES: *OCc1cncn1C(C)(C)C] | GS-2 | LC-MS: 637.2 [M + H]⁺; ¹HNMR (CD₃OD, 400 MHz): δ 1.62 (s, 9H), 4.11 (s, 2H), 4.34 (s, 2H), 5.40 (s, 2H), 6.20 (s, 1H), 6.51 (s, 2H), 7.02-7.04 (d, 2H), 7.16-7.25 (m, 3H), 7.31-7.33 (d, 2H), 7.42 (s, 1H), 7.54-7.55 (d, 2H), 7.60 (s, 1H), 7.76-7.80 (d, 1H), 8.10 (br, 1H), 8.29-8.31 (d, 1H), 8.81 (s,1H). |
| 30 | 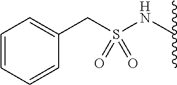 [SMILES: *NS(=O)(=O)Cc1ccccc1] | 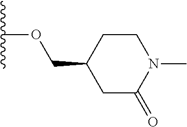 [SMILES: *OC[C@H]1CCN(C)C(=O)C1] | GS-3 | LC-MS: 626 [M + H]⁺. |
| 31 | 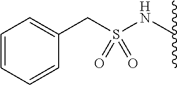 [SMILES: *NS(=O)(=O)Cc1ccccc1] | 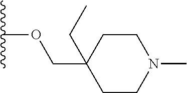 [SMILES: *OCC1(CC)CCN(C)CC1] | GS-2 | LC-MS: 638.2[M − H]⁻; ¹HNMR (CD₃OD, 400 MHz): δ 1.20-1.60 (m, 6H), 2.60-2.90 (m, 6H), 3.00-3.15 (m, 4H), 4.00 (s, 1H), 4.20-4.27 (m, 5H), 6.38 (s, 1H), 6.55 (s, 2H), 6.95-6.97 (d, 2H), 7.20-7.308 (m, 5H), 7.60-7.80 (m, 4H), 8.30-8.35 (m, 2H). |
| 32 | 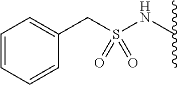 [SMILES: *NS(=O)(=O)Cc1ccccc1] | 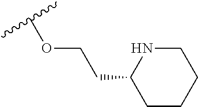 [SMILES: *OCC[C@H]1CCCCN1] | GS-2 | LC-MS: 612.4 [M + H]⁺; ¹HNMR (CD₃OD, 400 MHz): δ 1.25-1.50 (m, 4H), 1.70-1.95 (m, 4H), 2.55-2.60 (m, 1H), 2.80 (d, 2H), 3.20 (d, 1H), 4.12 (s, 2H), 4.29-4.36 (m, 4H), 6.27 (s, 1H), 6.57 (s, 2H), 7.05-7.07 (d, 2H), 7.21-7.27 (m, 2H), 7.31-7.33 (d, 1H), 7.37 (s, 1H), |
| | | | | 7.56-7.59 (m, 2H), 7.63 (s, 1H), 7.79- |
| | | | | 7.81 (m, 1H) 8.24-8.26 (d, 1H) 8.30-8.32 |
| | | | | (d, 1H). |
| 33 | 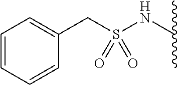 [SMILES: *NS(=O)(=O)Cc1ccccc1] | 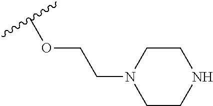 [SMILES: *OCCN1CCNCC1] | GS-2 | LC-MS: 613.4 [M + H]⁺; ¹HNMR (DMSO-d₆, 400 MHz): δ 3.10-3.32 (m, 4H), 3.50-3.55 (m, 4H), 4.26-4.32 (m, 8H), 6.21 (s, 1H), 6.46 (s, 2H), 7.08-7.09 (m, 2H), 7.26-7.33 (m, 3H), 7.38-7.41 (m, 1H), 7.50 (s, 1H), 7.58-7.61 (m, 2H), 7.70-7.75 (m, 1H), 7.85-7.95 (m, 1H), |
| | | | | 8.23-8.35 (m, 5H), 9.79 (s, 1H). |
| 34 | 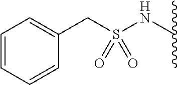 [SMILES: *NS(=O)(=O)Cc1ccccc1] | 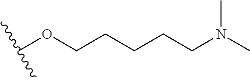 [SMILES: *OCCCCCN(C)C] | GS-2 | LC-MS: 614.2 [M + H]⁺; ¹HNMR (CD₃OD, 400 MHz): δ 1.13-1.19 (m, 2H), 1.45-1.57 (m, 4H), 2.77 (s, 6H), 2.83- 2.87 (t, 2H), 4.19 (s, 2H), 4.22-4.25 (t, 2H), 4.35 (s, 2H), 6.26 (s, 1H), 6.56 (s, 2H), 7.21-7.33 (m, 6H), 7.42 (s, 1H), |
| | | | | 7.59-7.60 (m, 3H), 7.82-7.84 (m, 1H), |
| | | | | 8.25-8.32 (m, 2H). |
| 35 | 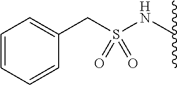 [SMILES: *NS(=O)(=O)Cc1ccccc1] | 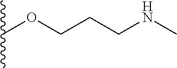 [SMILES: *OCCCNC] | GS-2 | LC-MS: 572.4 [M + H]⁺; ¹HNMR (CD₃OD, 400 MHz): δ 1.95-1.99 (m, 2H), 2.52 (s, 3H), 2.87-2.91 (t, 2H), 4.14 (s, 2H), 4.28-4.31 (t, 2H), 4.35 (s, 2H), 6.30 (s, 1H), 6.57 (s, 2H), 7.07-7.08 (d, 2H), 7.22-7.28 (m, 3H), 7.31-7.33 (d, 1H), |
| | | | | 7.37 (s, 1H), 7.57-7.59 (m, 2H), 7.63 (s, |
| | | | | 1H), 7.79-7.82 (m, 1H), 8.24-8.26 (m, |
| | | | | 1H), 8.30-8.32 (d, 1H). |
| 36 | 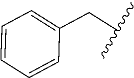 [SMILES: *Cc1ccccc1] | 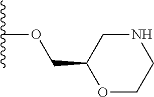 [SMILES: *OC[C@H]1CNCCO1] | GS-3 | LC-MS: 601 [M + H]⁺. |
| 37 | 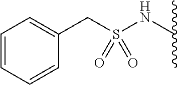 [SMILES: *NS(=O)(=O)Cc1ccccc1] | 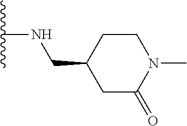 [SMILES: *NC[C@H]1CCN(C)C(=O)C1] | GS-3 | LC-MS: 625 [M + H]⁺. |
| 38 | 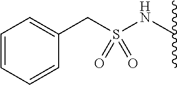 [SMILES: *NS(=O)(=O)Cc1ccccc1] | 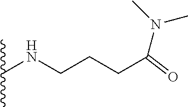 [SMILES: *NCCCC(=O)N(C)C] | GS-3 | LC-MS: 613 [M + H]⁺. |
| 39 | 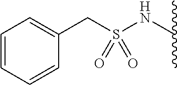 [SMILES: *NS(=O)(=O)Cc1ccccc1] | 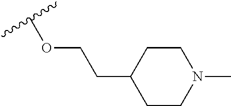 [SMILES: *OCCC1CCN(C)CC1] | GS-2 | LC-MS: 626.4 [M + H]⁺; ¹HNMR (CD₃OD, 400 MHz): δ 1.09 (bs, 2H), 1.20 (s, 1H), 1.36 (s, 2H), 1.54-1.56 (d, 2H), 2.46 (bs, 2H), 2.64 (s, 3H), 3.21 (s, 2H), 4.10 (s, 2H), 4.18-4.21 (t, 2H), 4.25 (s, 2H), 6.14 (s, 1H), 6.47 (s, 2H), 7.00-7.02 (d, 2H), 7.13-7.16 (t, 2H), 7.18-7.20 (d, |
| | | | | 1H), 7.23-7.25 (d, 1H), 7.31 (s, 1H), |
| | | | | 7.51-7.53 (d, 3H), 7.72-7.74 (m, 1H), |
| | | | | 8.17-8.19 (d, 1H), 8.21-8.23 (d, 1H). |
| 40 | 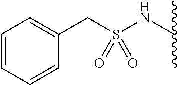 [SMILES: *NS(=O)(=O)Cc1ccccc1] | 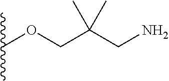 [SMILES: *OCC(C)(C)CN] | GS-3 | LC-MS: 586 [M + H]⁺. |
| 41 | 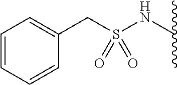 [SMILES: *NS(=O)(=O)Cc1ccccc1] | 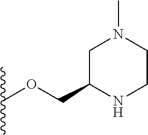 [SMILES: *OC[C@H]1CN(C)CCN1] | GS-2 | LC-MS: 613.4 [M + H]⁺; ¹HNMR (CD₃OD, 400 MHz): δ 2.92 (s, 3H), 3.41- 3.47 (m, 1H), 3.51-3.61 (m, 2H), 3.71- 3.77 (m, 3H), 4.09-4.12 (m, 1H), 4.16 (s, 2H), 4.34 (s, 2H), 4.47-4.51 (dd, 1H), 4.55-4.59 (dd, 1H), 6.33 (s, 1H), 6.55- 6.56 (d, 2H), 7.07-7.08 (d, 2H), 7.20-7.29 (m, 3H), 7.32-7.34 (m, 2H), 7.537.593 (m, 2H), 7.77-7.80 (m, 1H), 7.96 (s, 1H), |
| | | | | 8.24-8.26 (dd, 1H), 8.33-8.35 (d, 1H). |
| 42 | 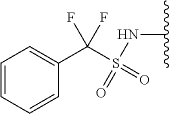 [SMILES: *NS(=O)(=O)C(F)(F)c1ccccc1] | 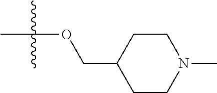 [SMILES: *OCC1CCN(C)CC1] | GS-2 | LC-MS: 648.1 [M + H]⁺; ¹HNMR (CD₃OD, 400 MHz): δ 1.34-1.37 (m, 2H), 1.70-1.74 (d, 3H), 2.61-2.64 (t, 2H), 2.760 (s, 3H), 3.0 (bs, 1H), 3.33-3.34 (d, 1H), 4.08-4.10 (d, 2H), 4.36 (s, 2H), 6.33 (s, 1H), 6.57 (s, 2H), 7.32-7.34 (d, 2H), 7.45-7.49 (m, 4H), 7.56-7.66 (m, 4H), 7.84-7.86 (d, 1H), 8.26-8.28 (d,1H), |
| | | | | 8.32-8.34 (d,1H). |
| 43 | 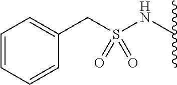 [SMILES: *NS(=O)(=O)Cc1ccccc1] | 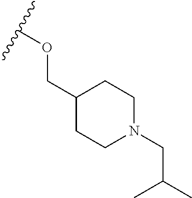 [SMILES: *OCC1CCN(CC(C)C)CC1] | GS-3 | LC-MS: 654 [M + H]⁺. |
| 44 | 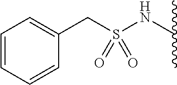 [SMILES: *NS(=O)(=O)Cc1ccccc1] | 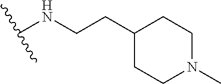 [SMILES: *NCCC1CCN(C)CC1] | GS-2 | LC-MS: 625.4 [M + H]⁺; ¹HNMR (CD₃OD, 400 MHz): δ 1.21-1.23 (m, 3H), 1.37-1.39 (m, 2H), 1.78-1.84 (m, 2H), 2.35 (m, 2H), 2.66 (s, 3H), 3.23-3.33 (m, 4H), 4.20 (s, 2H), 4.36 (s, 2H), 6.30 (s, 1H), 6.58 (s, 2H), 7.06-7.08 (d, 2H), |
| | | | | 7.18-7.22 (t, 2H), 7.25-7.32 (m, 3H), |
| | | | | 7.39 (d, 1H), 7.56-7.59 (m, 2H), 7.78- |
| | | | | 7.82 (m, 1H), 8.24-8.26 (m, 2H). |
| 45 | 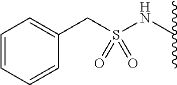 [SMILES: *NS(=O)(=O)Cc1ccccc1] | 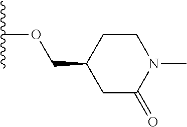 [SMILES: *OC[C@H]1CCN(C)C(=O)C1] | GS-2 | LC-MS: 626.3 [M + H]⁺; ¹HNMR (CD₃OD, 400 MHz): δ 1.26-1.31 (m, 2H), 1.59-1.63 (m, 1H), 1.86-1.92 (m, 2H), 2.21-2.24 (d, 1H), 2.79 (s, 3H), 2.99-306 (m, 2H), 4.10-4.11 (d, 2H), 4.20 (s, 2H), 4.35 (s, 2H), 6.27 (s, 1H), 6.55 (s, 2H), 7.09-7.10 (d, 2H), 7.21-7.28 (m, 3H), 7.31-7.33 (d, 1H), 7.56-7.59 (m, 2H), |
| | | | | 7.62 (s, 1H), 7.81-7.83 (m, 1H), 8.23- |
| | | | | 8.25 (m, 1H), 8.30-8.32 (d, 1H). |
| 46 | 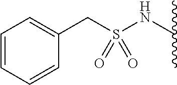 [SMILES: *NS(=O)(=O)Cc1ccccc1] | 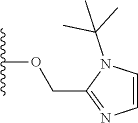 [SMILES: *OCc1nccn1C(C)(C)C] | GS-2 | LC-MS: 638.2 [M + H]⁺; ¹HNMR (CD₃OD, 400 MHz): δ 1.63 (s, 9H), 4.11 (s, 2H), 4.35 (s, 2H), 5.62 (s, 2H), 6.25- 6.26 (d, 1H), 7.03-7.05 (d, 2H), 7.17-7.24 (m, 3H), 7.32-7.34 (m, 3H), 7.53-7.55 (m, 2H), 7.62 (d, 2H), 7.69 (s, 1H), 7.76- 7.78 (m, 2H), 8.16-8.18 (m, 1H), 8.31- 8.33 (d, 1H). |
| 47 | 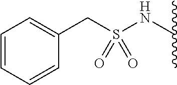 [SMILES: *NS(=O)(=O)Cc1ccccc1] | 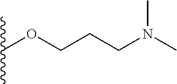 [SMILES: *OCCCN(C)C] | GS-2 | LC-MS: 586.4 [M + H]⁺; ¹HNMR (CD₃OD, 400 MHz): δ 1.95-1.99 (m, 2H), 2.65-2.66 (m, 6H), 2.93-2.97 (m, 2H), 4.15-4.17 (m, 2H), 4.28-4.31 (m, 2H), 4.34 (s, 2H), 6.30 (s, 1H), 6.56 (s, 2H), 7.07-7.08 (d, 2H), 7.22-7.33 (m, 4H), |
| | | | | 7.37 (s, 1H), 7.56-7.59 (m, 2H), 7.64 (s, |
| | | | | 1H), 7.80-7.82 (m, 1H), 8.25-8.27 (d, |
| | | | | 1H), 8.30-8.32 (d, 1H). |
| 48 | 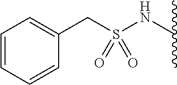 [SMILES: *NS(=O)(=O)Cc1ccccc1] | 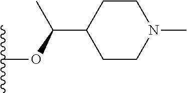 [SMILES: *O[C@@H](C)C1CCN(C)CC1] | GS-2 | LC-MS: 626.4 [M + H]⁺; ¹HNMR (CD₃OD, 400 MHz): δ 1.15-1.17 (d, 2H), 1.290-1.43 (m, 3H), 1.60 (m, 1H), 1.73- 1.76 (bs, 1H), 2.03 (m, 1H), 2.17-2.21 (m, 1H), 2.62-2.68 (t, 1H), 2.77 (s, 2H), 3.34 (s, 3H), 3.37 (m, 1H), 4.19 (s, 2H), |
| | | | | 4.36 (s, 2H), 6.27 (s, 1H), 6.51-6.60 (dd, |
| | | | | 2H), 7.09-7.10 (d, 2H), 7.22-7.27 (m, |
| | | | | 3H), 7.31-7.34 (m, 1H), 7.38 (s, 1H), |
| | | | | 7.58-7.60 (t, 3H), 7.81-7.83 (m, 1H), |
| | | | | 8.24-8.26 (m, 1H), 8.30-8.32 (d, 1H). |
| 49 | 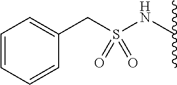 [SMILES: *NS(=O)(=O)Cc1ccccc1] | 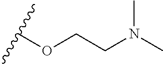 [SMILES: *OCCN(C)C] | GS-2 | LC-MS: 572.4 [M + H]⁺; ¹HNMR (CD₃OD, 300 MHz): δ 2.88 (s, 6H), 3.42- 3.44 (t, 2H), 4.17 (s, 2H), 4.36 (s, 2H), 4.54-4.57 (t, 2H), 6.37 (s, 1H), 6.58 (s, 2H), 7.10-7.17 (m, 2H), 7.23-7.38 (m, 5H), 7.58-7.61 (m, 2H), 7.73 (s, 1H), |
| | | | | 7.81-7.83 (d, 1H), 8.26-8.33 (m, 2H). |
| 50 | 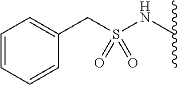 [SMILES: *NS(=O)(=O)Cc1ccccc1] | 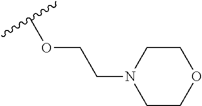 [SMILES: *OCCN1CCOCC1] | GS-3 | LC-MS: 614.4 [M + H]⁺. |
| 51 | 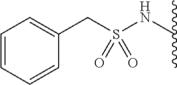 [SMILES: *NS(=O)(=O)Cc1ccccc1] | 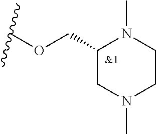 [SMILES: *OC[C@H]1CN(C)CCN1C] | GS-2 | LC-MS: 627.5 [M + H]⁺; ¹HNMR (CD₃OD, 400 MHz): δ 2.50 (s, 3H), 2.69 (s, 3H), 2.86-2.89 (m, 3H), 3.0 (d, 1H), 3.13 (d, 1H), 3.34 (s, 1H), 3.38-3.39 (d, 1H), 4.17 (s, 2H), 4.29-4.30 (d, 3H), 4.53-4.54 (m, 1H), 6.29 (s, 1H), 6.57 (s, 2H), 7.08-7.09 (d, 2H), 7.20-7.28 (m, 3H), 7.33-7.34 (d, 1H), 7.40 (s, 1H), 7.58-7.61 (m, 2H), 7.77 (s, 1H), 7.81- |
| | | | | 7.83 (m, 1H), 8.25-8.26 (d, 1H), 8.30- |
| | | | | 8.31 (d, 1H). |
| 52 | 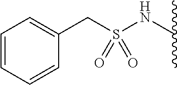 [SMILES: *NS(=O)(=O)Cc1ccccc1] | 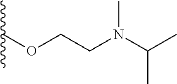 [SMILES: *OCCN(C)C(C)C] | GS-3 | LC-MS: 600 [M + H]⁺. |
| 53 | 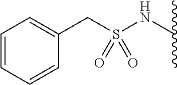 [SMILES: *NS(=O)(=O)Cc1ccccc1] | 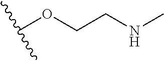 [SMILES: *OCCNC] | GS-2 | LC-MS: 558.3 [M + H]⁺; ¹HNMR (CD₃OD, 400 MHz): δ 2.69 (s, 3H), 3.29- 3.32 (m, 2H), 4.16 (s, 2H), 4.35 (s, 2H), 4.45-4.48 (m, 2H), 6.34 (s, 1H), 6.56 (s, 2H), 7.07-7.09 (d, 2H), 7.23-7.26 (m, 3H), 7.32-7.34 (d, 1H), 7.37 (s, 1H), |
| | | | | 7.56-7.58 (m, 2H), 7.73 (s, 1H), 7.97- |
| | | | | 8.01 (m, 1H), 8.22-8.24 (d, 1H), 8.30- |
| | | | | 8.32 (d, 1H). |
| 54 | 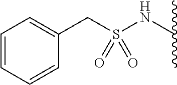 [SMILES: *NS(=O)(=O)Cc1ccccc1] | 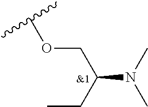 [SMILES: *OC[C@H](CC)N(C)C] | GS-3 | LC-MS: 600 [M + H]⁺ |
| 55 | 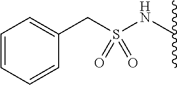 [SMILES: *NS(=O)(=O)Cc1ccccc1] | 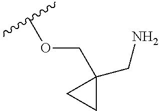 [SMILES: *OCC1(CN)CC1] | GS-3 | LC-MS: 584 [M + H]⁺. |
| 56 | 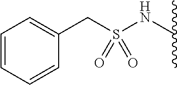 [SMILES: *NS(=O)(=O)Cc1ccccc1] | 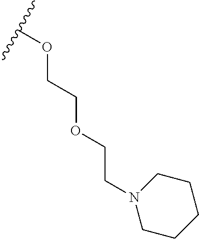 [SMILES: *OCCOCCN1CCCCC1] | GS-3 | LC-MS: 656 [M + H]⁺. |
| 57 | 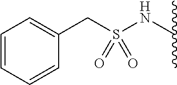 [SMILES: *NS(=O)(=O)Cc1ccccc1] | 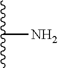 [SMILES: *N] | | LC-MS: 500.3 [M + H]⁺; ¹HNMR (CD₃OD, 300 MHz): δ 4.21 (s, 2H), 4.32 (s, 2H), 6.38 (s, 1H), 6.57 (s, 2H), 7.07- 7.08 (d, 2H), 7.23-7.29 (m, 4H), 7.37- 7.39 (m, 2H), 7.52-7.55 (m, 2H), 7.76- 7.79 (m, 1H) 8.21-8.24 (m, 2H). |
| 58 | 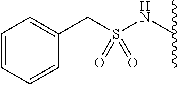 [SMILES: *NS(=O)(=O)Cc1ccccc1] | 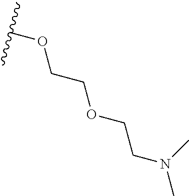 [SMILES: *OCCOCCN(C)C] | GS-3 | LC-MS: 616 [M + H]⁺. |
| 59 | 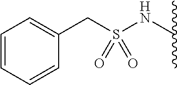 [SMILES: *NS(=O)(=O)Cc1ccccc1] | 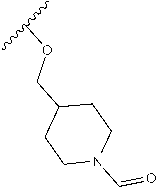 [SMILES: *OCC1CCN(C=O)CC1] | GS-2 | LC-MS: 626.4 [M + H]⁺; ¹HNMR (CD₃OD, 400 MHz): δ 0.86-0.97 (m, 2H), 1.40-1.49 (m, 2H), 4.65-1.69 (m, 2H), 2.24-2.31 (m, 1H), 2.72-2.79 (m, 1H), 3.44-3.47 (bs, 1H), 4.01-4.10 (m, 3H), 4.16 (s, 2H), 4.33 (s, 2H), 6.25-6.25 (bs, 1H), 6.53 (s, 2H), 7.06-7.08 (d, 1H), 7.19-7.31 (m, 4H), 7.42-7.43 (d, 1H), 7.54-7.59 (m, 3H), 7.80-7.82 (m, 2H), 8.22-8.24 (m, 1H), 8.28-8.30 (d, 1H). |
| 60 | 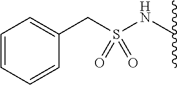 [SMILES: *NS(=O)(=O)Cc1ccccc1] | 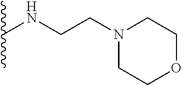 [SMILES: *NCCN1CCOCC1] | GS-2 | LC-MS: 613.4 [M + H]⁺; ¹HNMR (CD₃OD, 400 MHz): δ 2.94-3.02 (m, 3H), 3.14-3.20 (m, 4H), 3.42-3.53 (m, 2H), 2.61-2.69 (m, 2H), 3.78-3.84 (m, 2H), 4.21 (s, 2H), 4.38 (m, 2H), 6.41 (s, 1H), 6.59 (s, 2H), 7.09-7.11 (d, 2H), 7.21-7.28 |
| | | | | (m, 3H), 7.32-7.34 (m, 2H), 7.41 (s, 1H), |
| | | | | 7.56-7.58 (m, 2H), 8.45-8.52 (s, 1H), |
| | | | | 8.27-8.29 (d, 1H). |
| 61 | 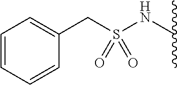 [SMILES: *NS(=O)(=O)Cc1ccccc1] | 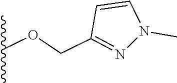 [SMILES: *OCc1ccn(C)n1] | | LC-MS: 595.3 [M + H]⁺; ¹HNMR (CD₃OD, 400 MHz): δ 3.64 (s, 3H), 4.11 (s, 2H), 4.31 (s, 2H), 5.14 (s, 2H), 6.18 (s, 1H), 6.51 (s, 2H), 7.16-7.28 (m, 5H), 7.43 (s, 1H), 7.52-7.56 (m, 3H), 7.78- 7.80 (m, 2H), 8.15-8.18 (m, 2H), 8.25- |
| | | | | 8.27 (m, 2H). |
| 62 | 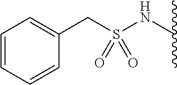 [SMILES: *NS(=O)(=O)Cc1ccccc1] | 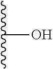 [SMILES: *O] | GS-2 | LC-MS: 501.2 [M + H]⁺; ¹HNMR (CD₃OD, 300 MHz): δ 4.21 (s, 2H), 4.32 (s, 2H), 6.23 (s, 1H), 6.57 (s, 2H), 7.06- 7.09 (d, 2H), 7.23-7.29 (t, 4H), 7.45 (s, 1H), 7.53-7.57 (t, 3H), 7.78-7.79 (t, 1H), 8.25-8.28 (t, 2H). |
| 63 | 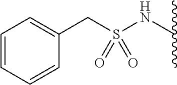 [SMILES: *NS(=O)(=O)Cc1ccccc1] | 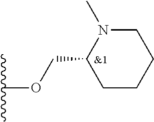 [SMILES: *OC[C@H]1CCCCN1C] | GS-2 | LC-MS: 611.7 [M + H]⁺; ¹HNMR (CD₃OD, 400 MHz): δ 1.48 (m, 1H), 1.79 (m, 5H), 2.85 (s, 3H), 2.99-3.00 (m, 1H), 3.43-3.48 (d, 1H), 4.15 (s, 2H), 4.35 (s, 3H), 4.54-4.59 (d, 1H), 4.77 (m, 1H), 6.35 (s, 1H), 6.55 (s, 2H), 7.07-7.09 (d, 2H), 7.23-7.25 (m, 3H), 7.32-7.34 (d, 2H), 7.56-7.58 (m, 2H), 7.78-7.79 (m, |
| | | | | 2H), 8.24-8.26 (d, 1H), 8.31-8.34 (d, |
| | | | | 1H). |
| 64 | 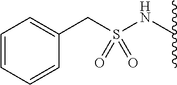 [SMILES: *NS(=O)(=O)Cc1ccccc1] | 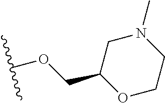 [SMILES: *OC[C@H]1CN(C)CCO1] | GS-2 | LC-MS: 613.7 [M + H]⁺; ¹HNMR(CD₃OD, 400 MHz): δ 2.66-2.73 (m, 2H), 2.71 (s, 3H), 3.29-3.36 (m, 2H), 3.56-3.59 (m, 1H), 3.82-3.89 (m, 2H), 4.15 (s, 2H), 4.30-4.32 (t, 2H), 4.33 (s, 2H), 6.26-6.26 (bs, 1H), 6.54 (s, 2H), 7.06-7.07 (d, 2H), 7.20-7.27 (m, 3H), |
| | | | | 7.30-7.32 (d, 1H), 7.39 (s, 1H), 7.56-7.60 |
| | | | | (m, 2H), 7.67 (s, 1H), 7.80-7.82 (m, 1H), |
| | | | | 8.24-8.26 (m, 1H), 8.29-8.31 (d, 1H). |
| 65 | 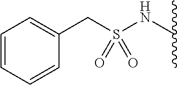 [SMILES: *NS(=O)(=O)Cc1ccccc1] | 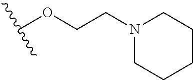 [SMILES: *OCCN1CCCCC1] | GS-3 | LC-MS: 612 [M + H]⁺. |
| 66 | 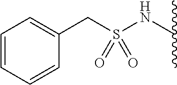 [SMILES: *NS(=O)(=O)Cc1ccccc1] | 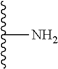 [SMILES: *N] | | LC-MS: 514.3 [M + H]⁺; ¹HNMR (CD₃OD, 400 MHz): δ 2.77 (s, 3H), 4.18 (s, 2H), 4.32 (s, 2H), 6.45 (s, 1H), 6.55 (s, 2H), 7.07-7.08 (d, 2H), 7.23-7.29 (m, 5H), 7.38 (s, 1H), 7.52-7.55 (m, 2H), 7.77-7.79 (m, 1H), 8.20-8.23 (m, 2H). |
| 67 | 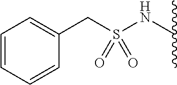 [SMILES: *NS(=O)(=O)Cc1ccccc1] | 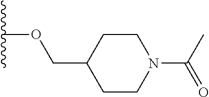 [SMILES: *OCC1CCN(C(C)=O)CC1] | GS-3 | LC-MS: 640 [M + H]⁺. |
| 68 | 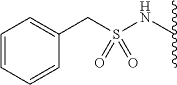 [SMILES: *NS(=O)(=O)Cc1ccccc1] | 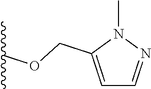 [SMILES: *OCc1ccnn1C] | GS-2 | LC-MS: 595.4 [M + H]⁺; ¹HNMR (CD₃OD, 400 MHz): δ 3.63 (s, 3H), 4.07 (s, 2H), 4.32 (s, 2H), 5.26 (s, 2H), 6.09 (s, 1H), 6.22 (s, 1H), 6.55 (s, 2H), 7.17- 7.30 (m, 5H), 7.40 (s, 1H), 7.54-7.59 (m, 3H), 7.78-7.81 (m, 2H), 8.18-8.20 (m, |
| | | | | 2H), 8.27-8.29 (m, 1H). |
| 69 | 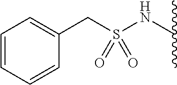 [SMILES: *NS(=O)(=O)Cc1ccccc1] | 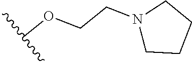 [SMILES: *OCCN1CCCC1] | GS-3 | LC-MS: 598.3 [M + H]⁺. |
| 70 | 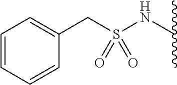 [SMILES: *NS(=O)(=O)Cc1ccccc1] | 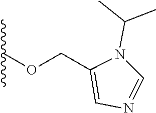 [SMILES: *OCc1cncn1C(C)C] | GS-2 | LC-MS: 621.2 [M − H]⁺; ¹HNMR (CD₃OD, 400 MHz): δ 1.47-1.48 (d, 6H), 4.12 (s, 2H), 4.36 (s, 2H), 4.65-4.72 (m, 1H), 5.40 (s, 2H), 6.28 (s, 1H), 6.56 (s, 2H), 7.05-7.07 (d, 2H), 7.19-7.28 (m, 3H), 7.32-7.36 (m, 2H), 7.41 (s, 1H), 7.57-7.61 (m, 3H), 7.80-7.82 (m, 1H), |
| | | | | 8.22-8.24 (m, 1H), 8.31-8.33 (d, 1H), |
| | | | | 8.98 (s, 1H). |
| 71 | 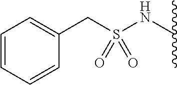 [SMILES: *NS(=O)(=O)Cc1ccccc1] | 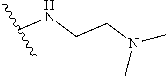 [SMILES: *NCCN(C)C] | GS-2 | LC-MS: 571.3 [M + H]⁺; ¹HNMR (CD₃OD, 400 MHz): δ 2.77 (s, 6H), 3.16- 3.19 (t, 2H), 3.58-3.61 (t, 2H), 4.19 (s, 2H), 4.34 (s, 2H), 6.45 (s, 1H), 6.56 (s, 2H), 7.08-7.10 (d, 2H), 7.20-7.27 (m, 3H), 7.29-7.31 (d, 1H), 7.33 (m, 2H), |
| | | | | 7.53-7.55 (m, 2H), 7.77-7.79 (m, 1H), |
| | | | | 8.20-8.23 (m, 1H), 8.24-8.26 (d, 1H). |
| 72 | 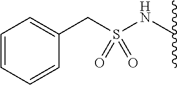 [SMILES: *NS(=O)(=O)Cc1ccccc1] | 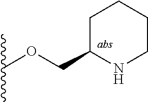 [SMILES: *OC[C@H]1CCCCN1] | GS-3 | LC-MS: 598 [M + H]⁺. |
| 73 | 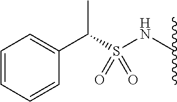 [SMILES: *NS(=O)(=O)[C@@H](C)c1ccccc1] | 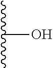 [SMILES: *O] | GS-2 | LC-MS: 515.1 [M + H]⁺; ¹HNMR (CD₃OD, 400 MHz): δ 1.58-1.59 (d, 3H), 4.16-4.18 (q, 1H), 4.32 (s, 2H), 6.26 (s, 1H), 6.53-6.58 (m, 2H), 7.12-7.14 (m, 2H), 7.21-7.23 (m, 2H), 7.26-7.28 (d, 1H), 7.30 (s, 1H), 7.52 (s, 1H), 7.53-7.55 (m, 3H), 7.74-7.76 (m, 1H), 8.21-8.23 |
| | | | | (m, 1H), 8.26-8.28 (d, 1H). |
| 74 | 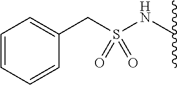 [SMILES: *NS(=O)(=O)Cc1ccccc1] | 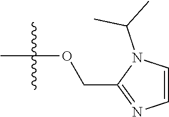 [SMILES: *OCc1nccn1C(C)C] | GS-2 | LC-MS: 621.2 [M − H]⁺; ¹HNMR (CD₃OD, 400 MHz): δ 1.38-1.40 (d, 6H), 4.11 (s, 2H), 4.37 (s, 2H), 4.73-4.77 (m, 1H), 5.52 (s, 2H), 6.29 (s, 1H), 6.54 (s, 2H), 7.05-7.07 (d, 2H), 7.21-7.29 (m, 3H), 7.34 (s, 1H), 7.36-7.37 (m, 2H), 7.56-7.59 (m, 2H), 7.64-7.64 (bs, 1H), 7.67 (s, 1H), 7.785-7.81 (m, 1H), 8.21- |
| | | | | 8.23 (m, 1H), 8.33-8.35 (d, 1H). |
| 75 | 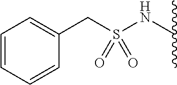 [SMILES: *NS(=O)(=O)Cc1ccccc1] | 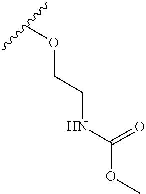 [SMILES: *OCCNC(=O)OC] | GS-2 | LC-MS: 602.0 [M + H]⁺; ¹HNMR (CD₃OD, 400 MHz): δ 3.51 (s, 3H), 4.19- 4.21 (m, 4H), 4.34 (s, 2H), 6.27 (s, 1H), 6.56 (s, 2H), 7.05-7.07 (d, 2H), 7.18-7.21 (t, 2H), 7.24-7.28 (d, 2H), 7.30-7.32 (d, 1H), 7.46 (s, 1H), 7.56-7.58 (m, 2H), 7.58-7.61 (s, 1H), 7.80-7.82 (m, 2H), 8.24-8.26 (d, 1H), 8.29-8.31 (d, 1H). |
| 76 | 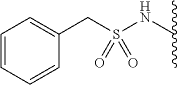 [SMILES: *NS(=O)(=O)Cc1ccccc1] | 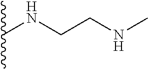 [SMILES: *NCCNC] | GS-2 | LC-MS: 556.6 [M + H]⁺; ¹HNMR (CD₃OD, 400 MHz): δ 2.58 (s, 3H), 3.05- 3.08 (t, 2H), 3.52-3.55 (t, 2H), 4.18 (s, 2H), 4.35 (s, 2H), 6.46 (s, 1H), 6.57 (s, 2H), 7.08-7.10 (d, 2H), 7.21-7.32 (m, 4H), 7.36 (s, 2H), 7.54-7.57 (m, 2H), |
| | | | | 7.78-7.81 (m, 1H), 8.22-8.24 (m, 1H), |
| | | | | 8.25-8.27 (d, 1H). |
| 77 | 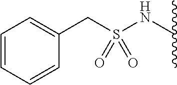 [SMILES: *NS(=O)(=O)Cc1ccccc1] | 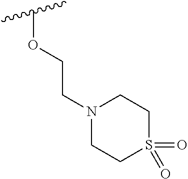 [SMILES: *OCCN1CCS(=O)(=O)CC1] | GS-3 | LC-MS: 662 [M + H]⁺. |
| 78 | 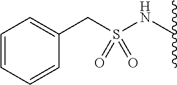 [SMILES: *NS(=O)(=O)Cc1ccccc1] | 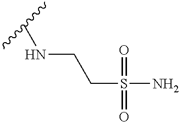 [SMILES: *NCCS(N)(=O)=O] | GS-3 | LC-MS: 607 [M + H]⁺. |
| 79 | 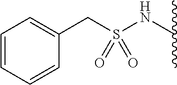 [SMILES: *NS(=O)(=O)Cc1ccccc1] | 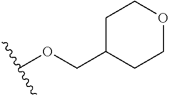 [SMILES: *OCC1CCOCC1] | GS-2 | LC-MS: 599.4 [M + H]⁺; ¹HNMR (CD₃OD, 400 MHz): δ 1.10-1.17 (m, 2H), 1.29-1.32 (m, 2H), 1.55-1.65 (m, 1H), 3.02-3.09 (t, 2H),3.69-3.73 (d, 2H), 4.01- 4.02 (d, 2H), 4.17 (s, 2H), 4.35 (s, 2H), 6.26 (s, 1H), 6.55 (s, 2H), 7.08-7.09 (d, 2H), 7.22-7.33 (m, 4H), 7.44 (s, 1H), |
| | | | | 7.56-7.61 (m, 3H), 7.81-7.84 (m, 1H), |
| | | | | 8.24-8.32 (m, 2H). |
| 80 | 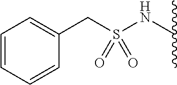 [SMILES: *NS(=O)(=O)Cc1ccccc1] | 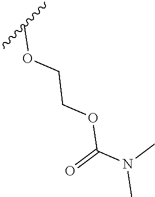 [SMILES: *OCCOC(=O)N(C)C] | GS-2 | LC-MS: 616.4 [M + H]⁺; ¹HNMR (CD₃OD, 400 MHz): δ 2.66 (s, 3H), 2.75 (s, 3H), 4.15-4.17 (m, 4H), 4.34 (s, 2H), 4.40-4.42 (m, 2H), 6.26 (s, 1H), 6.56 (s, 2H), 7.06-7.08 (d, 2H), 7.19-7.27 (m, 3H), 7.30-7.32 (d, 1H), 7.45-7.46 (d, 1H), 7.56-7.59 (m, 3H), 7.80-7.82 (m, 1H), 8.24-8.26 (m, 1H), 8.30-8.32 (d, 1H). |
| 81 | 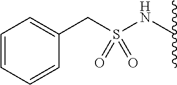 [SMILES: *NS(=O)(=O)Cc1ccccc1] | 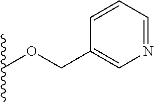 [SMILES: *OCc1cccnc1] | GS-2 | LC-MS: 592.3 [M + H]⁺; ¹HNMR (CD₃OD, 400 MHz): δ 4.09 (s, 2H), 4.33 (s, 2H), 5.32 (s, 2H), 6.21 (s, 1H), 6.51 (s, 2H), 7.00-7.02 (m, 2H), 7.15-7.24 (m, 3H), 7.29-7.31 (m, 1H), 7.37 (s, 1H), 7.44-7.47 (m, 1H), 7.52-7.57 (m, 2H), 7.66 (s, 1H), 7.77-7.79 (m, 1H), 7.90- |
| | | | | 7.92 (m, 1H), 8.15-8.17 (m, 1H), 8.28- |
| | | | | 8.30 (m, 1H), 8.46-8.47 (m, 1H), 8.53 |
| | | | | (bs, 1H). |
| 82 | 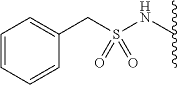 [SMILES: *NS(=O)(=O)Cc1ccccc1] | 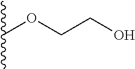 [SMILES: *OCCO] | GS-1 | LC-MS: 595.4 [M + H]⁺; ¹HNMR (CD₃OD, 400 MHz): δ 3.63 (s, 3H), 4.07 (s, 2H), 4.32 (s, 2H), 5.26 (s, 2H), 6.09 (s, 1H), 6.22 (s, 1H), 6.55 (s, 2H), 7.17- 7.30 (m, 5H), 7.40 (s, 1H), 7.54-7.59 (m, 3H), 7.78-7.81 (m, 2H), 8.18-8.20 (m, |
| | | | | 2H), 8.27-8.29 (m, 1H). |
| 83 | 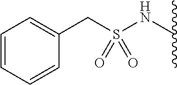 [SMILES: *NS(=O)(=O)Cc1ccccc1] | 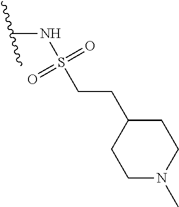 [SMILES: *NS(=O)(=O)CCC1CCN(C)CC1] | GS-2 | LC-MS: 689.3 [M + H]⁺; ¹HNMR (CD₃OD, 400 MHz): δ 1.21-1.30 (m, 2H), 1.43-1.52 (m, 2H), 1.53-1.59 (m, 2H), 1.66-1.69 (m, 2H), 2.68-2.79 (m, 5H), 3.25-3.34 (m, 4H), 4.19 (s, 2H), 4.36 (s, 2H), 6.41-6.46 (d, 3H), 7.07-7.09 (d, 2H), 7.19-7.27 (m, 3H), 7.32-7.34 (d, 1H), 7.42 (s, 1H), 7.47-7.54 (m, 3H), 7.76-7.78 (d, 1H), 8.13-8.15 (d, 1H), 8.27-8.29 (d, 1H). |
| 84 | 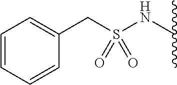 [SMILES: *NS(=O)(=O)Cc1ccccc1] | 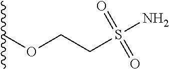 [SMILES: *OCCS(N)(=O)=O] | GS-3 | LC-MS: 608 [M + H]⁺. |
| 85 | 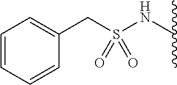 [SMILES: *NS(=O)(=O)Cc1ccccc1] | 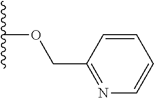 [SMILES: *OCc1ccccn1] | GS-2 | LC-MS: 592.2 [M + H]⁺; ¹HNMR (CD₃OD, 400 MHz): δ 4.12 (s, 2H), 4.33 (s, 2H), 5.30 (s, 2H), 6.24 (d, 1H), 6.52 (s, 2H), 7.01-7.03 (m, 2H), 7.14-7.21 (m, 3H), 7.24-7.31 (m, 3H), 7.38-7.39 (d, 1H), 7.51-7.54 (m, 2H), 7.63-7.68 (m, |
| | | | | 2H), 7.76-7.78 (m, 1H), 8.14-8.16 (m, |
| | | | | 1H), 8.30 (d, 1H), 8.36-8.37 (d, 1H). |
| 86 | 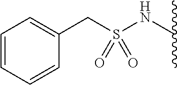 [SMILES: *NS(=O)(=O)Cc1ccccc1] | 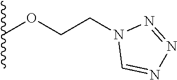 [SMILES: *OCCn1cnnn1] | GS-2 | LC-MS: 597.3 [M + H]⁺; ¹HNMR (CD₃OD, 400 MHz): δ 4.18 (s, 2H), 4.34 (s, 2H), 4.61-4.64 (t, 2H), 4.77-4.79 (t, 2H), 6.24 (s, 1H), 6.50 (s, 2H), 7.06-7.08 (d, 2H), 7.18-7.27 (m, 3H), 7.30-7.32 (d, 1H), 7.41 (bs, 1H), 7.52 (s, 1H), 7.54- |
| | | | | 7.59 (t, 2H), 7.79-7.82 (m, 1H), 8.20- |
| | | | | 8.22 (m, 1H), 8.29-8.32 (m, 1H), 9.14 (s, |
| | | | | 1H). |
| 87 | 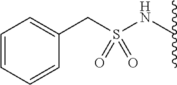 [SMILES: *NS(=O)(=O)Cc1ccccc1] | 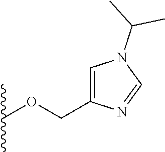 [SMILES: *OCc1cn(C(C)C)cn1] | GS-2 | LC-MS: 621.2 [M − H]⁻; ¹HNMR (CD₃OD, 400 MHz): δ 1.42-1.43 (d, 6H), 4.12 (s, 2H), 4.35 (s, 2H), 4.43-4.46 (m, 1H), 5.29 (s, 2H), 6.22 (s, 1H), 6.56 (s, 2H), 7.03-7.04 (d, 2H), 7.17-7.21 (t, 2H), 7.24-7.28 (m, 1H), 7.32-7.34 (d, 1H), 7.38 (s, 1H), 7.54 (s, 1H), 7.57-7.60 (m, 2H), 7.65 (s, 1H), 7.80-7.82 (m, 1H), 8.22-8.24 (m, 1H), 8.30-8.32 (d, 1H), 8.71 (s, 1H). |
| 88 | 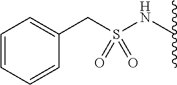 [SMILES: *NS(=O)(=O)Cc1ccccc1] | 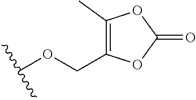 [SMILES: *OCc1oc(=O)oc1C] | GS-2 | LC-MS: 613.3 [M + H]⁺; ¹HNMR (CD₃OD, 400 MHz): δ 1.82 (s, 3H), 3.28 (s, 2H), 4.18 (s, 2H), 4.34 (s, 2H), 4.98 (s, 2H), 6.28 (s, 1H), 6.51 (s, 2H), 7.05- 7.10 (d, 2H), 7.20-7.31 (m, 4H), 7.47 (m, 1H), 7.52 (s, 2H), 7.62 (s, 1H), 7.81 (bs, 1H), 8.20 (bs, 1H), 8.28-8.32 (d, 1H). |
| 89 | 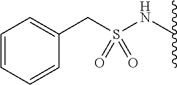 [SMILES: *NS(=O)(=O)Cc1ccccc1] | 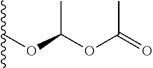 [SMILES: *O[C@@H](C)OC(C)=O] | GS-2 | LC-MS: 587.2 [M + H]⁺; ¹HNMR (CD₃OD, 400 MHz): δ 1.37-1.38 (d, 4H), 1.70 (s, 3H), 3.99 (s, 2H), 4.17 (s, 2H), 6.25 (s, 1H), 6.48 (s, 2H), 6.88-6.89 (d, 1H), 7.06-7.08 (d, 2H), 7.17-7.19 (m, 1H), 7.22-7.26 (m, 2H), 7.52-7.56 (m, |
| | | | | 3H), 7.73-7.82 (m, 2H), 8.18-8.20 (d, |
| | | | | 1H), 8.24-8.27 (d, 1H). |
| 90 | 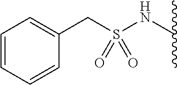 [SMILES: *NS(=O)(=O)Cc1ccccc1] | 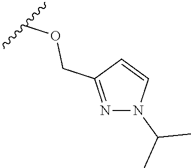 [SMILES: *OCc1ccn(C(C)C)n1] | GS-2 | LC-MS: 623.3 [M + H]⁺; ¹HNMR (CD₃OD, 400 MHz): δ 1.33-1.35 (s, 6H), 4.12 (s, 2H), 4.29-4.36 (m, 3H), 5.16 (s, 2H), 6.19 (s, 1H), 6.53 (s, 2H), 7.01-7.03 (m, 3H), 7.15-7.27 (m, 3H), 7.26-7.28 (d, 1H), 7.37 (bs, 1H), 7.44 (s, 1H), 7.53- 7.56 (m, 3H), 7.78-7.80 (m, 1H), 8.18- 8.20 (m, 1H), 8.25-8.27 (d, 1H). |
| 91 | 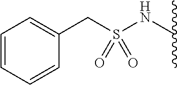 [SMILES: *NS(=O)(=O)Cc1ccccc1] | 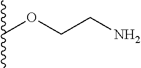 [SMILES: *OCCN] | GS-2 | LC-MS: 544.3 [M + H]⁺; ¹HNMR (CD₃OD, 400 MHz): δ 3.24-3.26 (t, 2H), 4.15 (s, 2H), 4.36 (s, 2H), 4.42-4.44 (t, 2H) 6.35 (s, 1H), 6.58 (s, 2H), 7.07-7.09 (d, 2H), 7.22-7.36 (m, 5H), 7.57-7.60 (m, 2H), 7.74 (s, 1H), 7.80-7.82 (m, 1H), |
| | | | | 8.24-8.33 (m, 2H). |
| 92 | 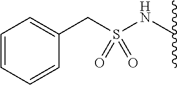 [SMILES: *NS(=O)(=O)Cc1ccccc1] | 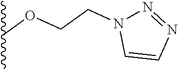 [SMILES: *OCCn1ccnn1] | GS-2 | LC-MS: 596.3 [M + H]⁺; ¹HNMR (CD₃OD, 400 MHz): δ 4.28 (s, 2H), 4.33 (s, 2H), 4.57-4.59 (m, 2H), 4.69-4.71 (m, 2H), 6.17 (s, 1H), 6.42 (s, 2H), 7.08-7.10 (d, 2H), 7.25-7.28 (m, 2H), 7.31-7.33 (d, 1H), 7.35-7.38 (d, 1H),7.40 (s, 1H), 7.53 |
| | | | | (s, 1H), 7.61-7.63 (m, 2H), 7.76 (s, 1H), |
| | | | | 7.90-7.92 (m, 1H), 8.20-8.24 (m, 2H), |
| | | | | 8.38-8.40 (d, 1H). |
| 93 | 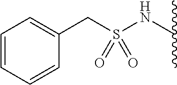 [SMILES: *NS(=O)(=O)Cc1ccccc1] | 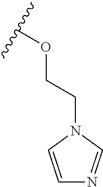 [SMILES: *OCCn1ccnc1] | GS-3 | LC-MS: 298 [M + H]⁺. |
| 94 | 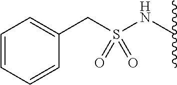 [SMILES: *NS(=O)(=O)Cc1ccccc1] | 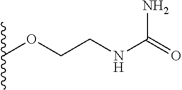 [SMILES: *OCCNC(N)=O] | GS-3 | LC-MS: 587 [M + H]⁺. |
| 95 | 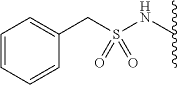 [SMILES: *NS(=O)(=O)Cc1ccccc1] | 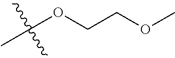 [SMILES: *OCCOC] | GS-2 | LC-MS: 559.3 [M + H]⁺; ¹HNMR (CD₃OD, 400 MHz): δ 3.11 (s, 3H), 3.42- 3.44 (d, 2H), 4.19 (s, 2H), 4.28-4.31 (m, 2H), 4.34 (s, 2H), 6.25 (s, 1H), 6.56 (s, 2H), 7.07-7.09 (d, 2H), 7.20-7.27 (m, 2H), 7.30-7.32 (d, 1H), 7.46 (s, 1H), |
| | | | | 7.56-7.59 (m, 2H), 7.60-7.61 (d, 1H), |
| | | | | 7.80-7.83 (d, 2H), 8.24-8.25 (d, 1H), |
| | | | | 8.29-8.31 (d, 1H). |
| 96 | 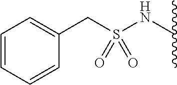 [SMILES: *NS(=O)(=O)Cc1ccccc1] | 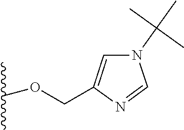 [SMILES: *OCc1cn(C(C)(C)C)cn1] | GS-2 | LC-MS: 636.8 [M + H]⁺; ¹HNMR (CD₃OD, 400 MHz): δ 1.51 (s, 9H), 4.10 (s, 2H), 4.35 (s, 2H), 5.30 (s, 2H), 6.22- 6.23 (bs, 1H), 6.56 (s, 2H), 7.02-7.04 (d, 2H), 7.17-7.21 (t, 2H), 7.24-7.28 (m, 1H), 7.32-7.34 (d, 1H), 7.38-7.38 (bs, 1H), 7.56-7.61 (m, 2H), 7.65 (s, 1H), 7.69-7.69 (bs, 1H), 7.79-7.82 (m, 1H), 8.22-8.25 (m, 1H), 8.30-8.32 (d, 1H), |
| | | | | 8.76 (s, 1H). |
| 97 | 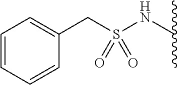 [SMILES: *NS(=O)(=O)Cc1ccccc1] | 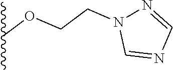 [SMILES: *OCCn1cncn1] | GS-2 | LC-MS: 596.3 [M + H]⁺; ¹HNMR (CD₃OD, 400 MHz): δ 4.17 (s, 2H), 4.34 (s, 2H), 4.49-4.51 (m, 2H), 4.56-4.58 (m, 2H), 6.25 (d, 1H), 6.51 (s, 2H), 7.06-7.07 (d, 2H), 7.18-7.28 (m, 3H), 7.30-7.32 (d, 1H), 7.42 (s, 1H), 7.51 (s, 1H), 7.56-7.60 |
| | | | | (m, 2H), 7.80-7.82 (m, 1H), 7.93 (s, 1H), |
| | | | | 8.21-8.23 (m, 1H), 8.28-8.30 (d, 1H), |
| | | | | 8.49 (s, 1H). |
| 98 | 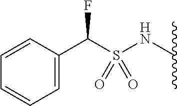 [SMILES: *NS(=O)(=O)[C@H](F)c1ccccc1] | 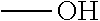 | GS-2 | LC-MS: 519.3 [M + H]⁺; ¹HNMR (CD₃OD, 400 MHz): δ 4.33 (s, 2H), 6.09 (s, 1H), 6.31 (s, 1H), 6.4 (s, 2H), 6.51 (s, 1H), 7.29 (d, 1H), 7.365-7.409 (m, 4H), 7.47 (s, 1H), 7.54-7.59 (m, 3H), 8.23- 8.24 (d, 1H), 8.24-8.25 (d, 1H), 8.29-8.30 (d, 1H). |
| 99 | 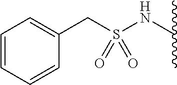 [SMILES: *NS(=O)(=O)Cc1ccccc1] | 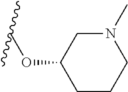 [SMILES: *O[C@H]1CCCN(C)C1] | GS-2 | LC-MS: 597.7 [M + H]⁺; ¹HNMR (CD₃OD, 400 MHz): δ 1.71-1.78 (m, 2H), 2.07-2.10 (m, 2H), 2.77 (s, 3H), 2.96-3.02 (t, 1H), 3.19-3.23 (d, 1H), 3.45-3.49 (d, 1H), 3.56-3.59 (d, 1H), 4.16 (s, 2H), 4.34 (s, 2H), 5.04 (s, 1H), 5.31 |
| | | | | (s, 1H), 6.42 (s, 1H), 6.49-6.61 (m, 2H), |
| | | | | 7.09-7.11 (d, 2H), 7.20-7.28 (m, 2H), |
| | | | | 7.30-7.32 (d, 1H), 7.35 (s, 1H), 7.55-7.59 |
| | | | | (m, 2H), 7.78-7.80 (s, 1H), 7.85 (s, 1H), |
| | | | | 8.23-8.25 (d, 1H), 8.29-8.31 (d, 1H). |
| 100 | 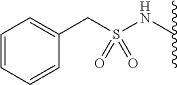 [SMILES: *NS(=O)(=O)Cc1ccccc1] | 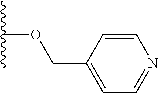 [SMILES: *OCc1ccncc1] | GS-2 | LC-MS: 592.1 [M + H]⁺; ¹HNMR (CD₃OD, 400 MHz): δ 4.16 (s, 2H), 4.36 (s, 2H), 5.53 (s, 2H), 6.24-6.28 (d, 1H), 6.53 (s, 2H), 7.02-7.06 (d, 2H), 7.17-7.22 (m, 3H), 7.34-7.36 (d, 2H), 7.52-7.54 (m, 2H), 7.78-7.80 (m, 2H), 7.82-7.84 (d, |
| | | | | 2H), 8.18-8.20 (d, 1H), 8.32-8.36 (d, |
| | | | | 1H), 8.58-8.62 (d, 2H). |
| 101 | 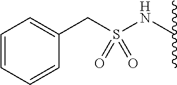 [SMILES: *NS(=O)(=O)Cc1ccccc1] | 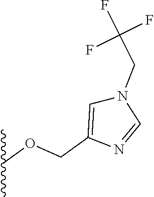 [SMILES: *OCc1cn(CC(F)(F)F)cn1] | GS-2 | LC-MS: 662.9 [M + H]⁺; ¹HNMR (DMSO-d₆, 400 MHz): δ 4.25-4.28 (m, 2H), 4.33 (s, 2H), 4.04 (bs, 2H), 4.95- 4.98 (m, 2H), 5.09 (s, 2H), 6.19 (s, 1H), 6.46 (s, 2H), 7.07-7.09 (d, 2H), 7.21-7.27 (m, 3H), 7.36-7.38 (d, 1H), 7.53 (s, 1H), 7.57-7.62 (m, 2H), 7.84 (bs, 1H), 7.89- 7.92 (m, 1H), 8.21-8.29 (m, 3H), 8.33- 8.36 (d, 1H) 9.77 (s, 1H). |
| 102 | 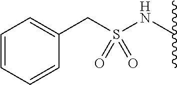 [SMILES: *NS(=O)(=O)Cc1ccccc1] | 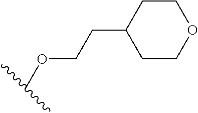 [SMILES: *OCCC1CCOCC1] | GS-2 | LC-MS: 611.2 [M + H]⁺; ¹HNMR (CD₃OD, 400 MHz): δ 0.99-1.09 (m, 2H), 1.24-1.41 (m, 5H), 3.06-3.12 (t, 2H), 3.70-3.74 (dd, 2H), 4.17 (s, 2H), 4.21- 4.25 (t, 2H), 4.34 (s, 2H), 6.27 (s, 1H), 6.56 (s, 2H), 7.07-7.09 (d, 2H), 7.21-7.28 (m, 3H), 7.30-7.32 (d, 1H), 7.45 (s, 1H), |
| | | | | 7.56-7.61 (m, 3H), 7.81-7.83 (m, 1H), |
| | | | | 8.24-8.27 (m, 1H), 8.29-8.32 (d, 1H). |
| 103 | 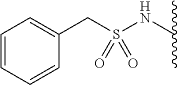 [SMILES: *NS(=O)(=O)Cc1ccccc1] | 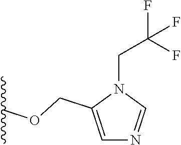 [SMILES: *OCc1cncn1CC(F)(F)F] | GS-2 | LC-MS: 660.9 [M + H]⁺; ¹HNMR (DMSO-d₆, 400 MHz): δ 4.25-4.32 (m, 4H), 5.05-5.09 (m, 2H), 5.26 (s, 2H), 6.17 (s, 1H), 6.46 (s, 2H), 7.04-7.11 (m, 3H), 7.22-7.29 (m, 3H), 7.38-7.40 (d, 1H), 7.53(s, 1H), 7.56 (s, 1H), 7.58-7.62 (m, 2H), 7.88-7.92 (m, 1H), 7.93-7.97 (m, 1H), 8.19-8.25 (m, 3H), 8.38 (d, 1H), 9.76 (s, 1H). |
| 104 | 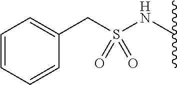 [SMILES: *NS(=O)(=O)Cc1ccccc1] | 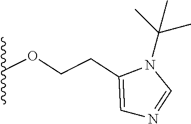 [SMILES: *OCCc1cncn1C(C)(C)C] | GS-2 | LC-MS: 651.1 [M + H]⁺; ¹HNMR (CD₃OD, 400 MHz): δ 1.55 (s, 9H), 3.12-3.16 (t, 2H), 4.11 (s, 2H), 4.33 (s, 2H), 4.52-4.55 (t, 2H), 6.26-6.26 (bs, 1H), 6.53 (s, 2H), 7.03-7.05 (d, 2H), 7.16-7.25 (m, 3H), 7.29-7.31 (d, 1H), 7.36-7.36 (d, 2H), 7.53-7.58 (m, 2H), 7.59 (s, 1H), 7.78-7.80 (q, 1H), 8.18-8.21 |
| | | | | (t, 1H), 8.27-8.29 (d, 1H), 8.74-8.75 (d, |
| | | | | 1H). |
| 105 | 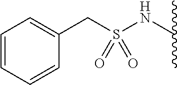 [SMILES: *NS(=O)(=O)Cc1ccccc1] | 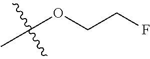 [SMILES: *OCCF] | GS-2 | LC-MS: 546.6 [M + H]⁺; ¹HNMR (CD₃OD, 400 MHz): δ 4.18 (s, 2H), 4.35 (s, 2H), 4.37-4.39 (m, 1H), 4.42-4.46 (m, 2H), 4.54 (m, 1H), 6.23 (s, 1H), 6.56 (s, 2H), 7.07-7.09 (d, 2H), 7.22-7.25 (m, 3H), 7.30-7.32 (d, 1H), 7.44-7.45 (bs, |
| | | | | 1H), 7.56-7.59 (m, 2H), 7.63 (s, 1H), |
| | | | | 8.30-8.32 (d, 1H), 7.81-7.83 (m, 1H), |
| | | | | 8.20-8.26 (m, 1H). |
| 106 | 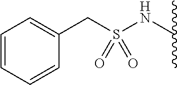 [SMILES: *NS(=O)(=O)Cc1ccccc1] | 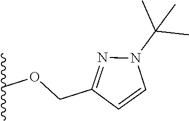 [SMILES: *OCc1ccn(C(C)(C)C)n1] | GS-2 | LC-MS: 637.0 [M + H]⁺; ¹HNMR (CD₃OD, 400 MHz): δ 1.43 (s, 9H), 4.12 (s,2H), 4.31 (s, 2H), 5.18 (s, 2H), 6.19 (s, 1H), 6.53 (s, 2H), 7.01-7.03 (d, 2H), 7.16-7.23 (m, 3H), 7.26-7.28 (d, 1H), 7.44-7.44 (s, 2H), 7.53-7.54 (d, 1H), 7.55 (s, 3H), 7.78-7.80 (m, 1H), 8.18-8.20 (m, 1H), 8.25-8.27 (d, 1H). |
| 107 | 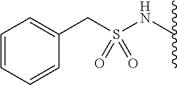 [SMILES: *NS(=O)(=O)Cc1ccccc1] | 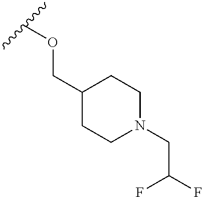 [SMILES: *OCC1CCN(CC(F)F)CC1] | GS-3 | LC-MS: 662 [M + H]⁺. |
| 108 | 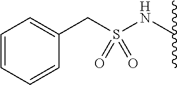 [SMILES: *NS(=O)(=O)Cc1ccccc1] | 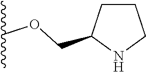 [SMILES: *OC[C@H]1CCCN1] | GS-3 | LC-MS: 584 [M + H]⁺. |
| 109 | 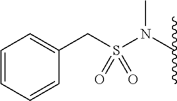 [SMILES: *N(C)S(=O)(=O)Cc1ccccc1] | 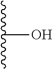 [SMILES: *O] | GS-2 | LC-MS: 515.3 [M + H]⁺; ¹HNMR (CD₃OD, 400 MHz): δ 2.99 (s, 3H), 4.16 (s, 2H), 4.32 (s, 2H), 6.32 (d, 1H), 6.56 (s, 2H), 7.14 (d, 2H), 7.25-7.30 (m, 4H), 7.53 (s, 2H), 7.58-7.64 (m, 2H), 7.86 (d, 1H), 8.25-8.29 (m, 2H). |
| 110 | 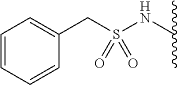 [SMILES: *NS(=O)(=O)Cc1ccccc1] | 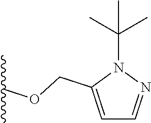 [SMILES: *OCc1ccnn1C(C)(C)C] | GS-2 | LC-MS: 637.2 [M + H]⁺; ¹HNMR (CD₃OD, 400 MHz): δ 1.53 (s, 9H), 4.09 (s, 2H), 4.32 (s, 2H), 5.40 (s, 2H), 6.16 (s, 1H), 6.25 (s, 1H), 6.53 (s, 2H), 7.02- 7.04 (d, 2H), 7.16-7.25 (m, 4H), 7.28- 7.30 (d, 1H), 7.38 (s, 1H), 7.53-7.55 (m, 3H), 7.77-7.79 (m, 1H), 8.17-8.18 (m, 1H), 8.27-8.29 (d, 1H). |
| 111 | 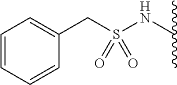 [SMILES: *NS(=O)(=O)Cc1ccccc1] | 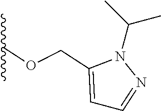 [SMILES: *OCc1ccnn1C(C)C] | GS-2 | LC-MS: 623 [M + H]⁺; ¹HNMR (CD₃OD, 400 MHz): δ 1.31-1.33 (d, 6H), 4.07 (s, 2H), 4.32 (s, 2H), 4.55-4.61 (m, 1H), 5.29 (s, 2H), 6.11-6.12 (d, 1H), 6.27 (s, 1H), 6.54 (s, 2H), 7.02-7.04 (d, 2H), 7.17-7.26 (m, 3H), 7.28-7.30 (s, 2H), 7.37 (s, 1H), 7.53-7.57 (m, 3H), 7.78- |
| | | | | 7.80 (m, 1H), 8.20-8.23 (m, 1H), 8.27- |
| | | | | 8.29 (d, 1H). |
| 112 | 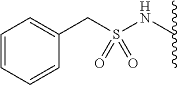 [SMILES: *NS(=O)(=O)Cc1ccccc1] | 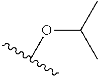 [SMILES: *OC(C)C] | GS-2 | LC-MS: 543.3 [M + H]⁺; ¹HNMR (CD₃OD, 400 MHz): δ 1.07-1.08 (d, 6H), 4.19 (s, 2H), 4.35 (s,2H), 5.04-5.07 (m, 1H), 6.25 (s, 1H), 6.55 (s, 2H), 7.08-7.10 (d, 2H), 7.22-7.32 (m, 4H), 7.45 (s, 1H), 7.56-7.60 (m, 3H), 7.81-7.83 (m, 1H), |
| | | | | 8.24-8.30 (m, 2H). |
| 113 | 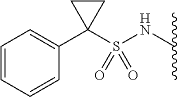 [SMILES: *NS(=O)(=O)C1(c2ccccc2)CC1] | 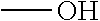 | GS-2 | LC-MS: 527.3 [M + H]⁺; ¹HNMR (CD₃OD, 400 MHz): δ 0.90-1.10 (t, 2H), 1.35-1.45 (t, 2H), 4.35 (s, 2H), 6.29 (s, 1H), 6.57 (s, 2H), 7.19-7.30 (m, 7H), 7.53-7.56 (m, 3H), 7.71-7.72 (d, 1H), 8.22-8.24 (dd, 1H), 8.28-8.29 (dd, 1H) |
| 114 | 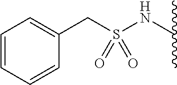 [SMILES: *NS(=O)(=O)Cc1ccccc1] | 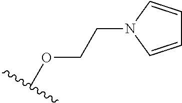 [SMILES: *OCCn1cccc1] | GS-3 | LC-MS: 554 [M + H]⁺. |
| 115 | 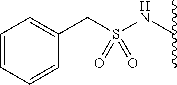 [SMILES: *NS(=O)(=O)Cc1ccccc1] | 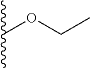 [SMILES: *OCC] | GS-1 | LC-MS: 529.1 [M + H]⁺; ¹HNMR (CD₃OD, 400 MHz): δ 1.10-1.14 (t, 3H), 4.17-4.22 (m, 4H), 4.34 (s, 2H), 6.25 (s, 1H), 6.55 (s, 2H), 7.06-7.08 (d, 2H), 7.20-7.27 (m, 3H), 7.29-7.31 (d, 1H), 7.44 (s, 1H), 7.55-7.58 (m, 3H), 7.79- |
| | | | | 7.81 (m, 1H), 8.23-8.25 (m, 1H), 8.28- |
| | | | | 8.30 (d, 1H). |
| 116 | 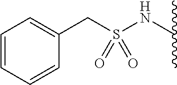 [SMILES: *NS(=O)(=O)Cc1ccccc1] | 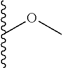 [SMILES: *OC] | GS-2 | LC-MS: 515.3 [M + H]⁺; ¹HNMR (CD₃OD, 400 MHz): δ 3.76 (s, 3H), 4.18 (s, 2H), 4.33 (s,2H), 6.25 (s, 1H), 6.55 (s, 2H), 7.05-7.07 (d, 2H), 7.20-7.31 (m, 4H), 7.44 (s, 1H), 7.56-7.58 (m, 3H), 7.79-7.81 (m, 1H), 8.23-8.30 (m, 2H). |
| 117 | 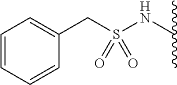 [SMILES: *NS(=O)(=O)Cc1ccccc1] | 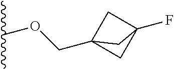 [SMILES: *OCC12CC(F)(C1)C2] | GS-2 | LC-MS: 599.3 [M + H]⁺; ¹HNMR (CD₃OD, 400 MHz): δ 1.79-1.80 (d, 6H), 4.15 (s, 2H), 4.33 (s, 2H), 4.39 (s, 2H), 6.22 (s, 1H), 6.53 (s, 2H), 7.05-7.07 (d, 2H), 7.19-7.27 (m, 3H), 7.29-7.31 (d, 1H), 7.40-7.41 (d, 1H), 7.53-7.58 (m, |
| | | | | 3H), 7.78-7.80 (m, 1H), 8.21-8.23 (m, |
| | | | | 1H), 8.29 (d, 1H). |
| 118 | 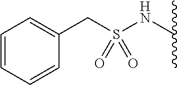 [SMILES: *NS(=O)(=O)Cc1ccccc1] | 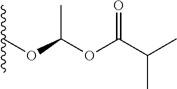 [SMILES: *O[C@@H](C)OC(=O)C(C)C] | GS-2 | LC-MS: 615.2 [M + H]⁺; ¹HNMR (CD₃OD, 400 MHz): δ 0.84-0.95 (m, 6H), 1.38-1.40 (d, 3H), 2.18-2.20 (m, 1H), 4.20 (s, 2H), 4.36 (s, 2H), 6.22 (s, 1H), 6.55 (s, 2H), 6.88-6.90 (m, 1H), 7.11- 7.13 (d, 2H), 7.25-7.28 (m, 3H), 7.31- |
| | | | | 7.33 (d, 1H), 7.64 (s, 1H), 7.56-7.59 (m, |
| | | | | 2H), 7.62 (s, 1H), 7.80-7.82 (m, 1H), |
| | | | | 8.23-8.25 (m, 1H), 8.31-8.33 (d, 1H). |
| 119 | 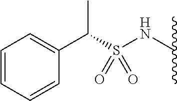 [SMILES: *NS(=O)(=O)[C@@H](C)c1ccccc1] | 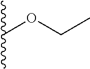 [SMILES: *OCC] | GS-2 | LC-MS: 543.4 [M + H]⁺; ¹HNMR (CD₃OD, 400 MHz): δ 1.12-1.15 (t, 3H), 1.56-1.58 (d, 3H), 4.1-4.15 (q, 1H), 4.18- 4.23 (m, 3H), 4.34 (s, 2H), 6.28 (s, 1H), 6.48-6.6 (q, 2H), 7.11 (d, 2H), 7.20-7.314 (m, 4H), 7.54-7.56 (m, 3H), 7.75-7.77 (m, 1H), 8.29-8.30 (d, 2H). |
| 120 | 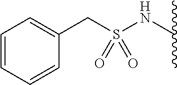 [SMILES: *NS(=O)(=O)Cc1ccccc1] | 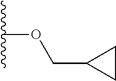 [SMILES: *OCC1CC1] | GS-2 | LC-MS: 555.4 [M + H]⁺; ¹HNMR (CD₃OD, 400 MHz): δ 0.07-0.10 (m, 2H), 0.30-0.33 (m, 2H), 0.85-0.93 (m, 1H), 3.97-3.99 (d, 2H), 4.19 (s, 2H), 4.35 (s, 2H), 6.25 (s, 1H), 6.57 (s, 2H), 7.07-7.09 (d, 2H), 7.21-7.32 (m, 4H), 7.45 (s, 1H), |
| | | | | 7.56-7.60 (m, 3H), 7.80-7.82 (m, 1H), |
| | | | | 8.24-8.31 (m, 2H). |
| 121 | 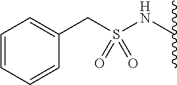 [SMILES: *NS(=O)(=O)Cc1ccccc1] | 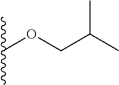 [SMILES: *OCC(C)C] | GS-2 | LC-MS: 557.4 [M + H]⁺; ¹HNMR (CD₃OD, 400 MHz): δ 0.81-0.83 (d, 6H), 1.81-1.84 (m, 1H), 3.95-3.97 (d, 2H), 4.18 (s, 2H), 4.34 (s, 2H), 6.27 (s, 1H), 6.56 (s, 2H), 7.07-7.09 (d, 2H), 7.21-7.32 (m, 4H), 7.44 (s, 1H), 7.56-7.58 (m, 3H), |
| | | | | 7.80-7.82 (m, 1H), 8.23-8.31 (m, 2H). |
| 122 | 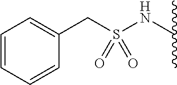 [SMILES: *NS(=O)(=O)Cc1ccccc1] | 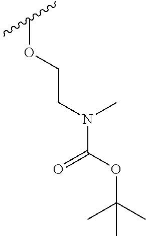 [SMILES: *OCCN(C)C(=O)OC(C)(C)C] | GS-2 | LC-MS: 658.4 [M + H]⁺; ¹HNMR (CD₃OD, 400 MHz): δ 1.31-1.36 (m, 9H), 2.82 (s, 3H), 3.45-3.48 (t, 2H), 4.19 (s, 2H), 4.29-4.31 (t, 2H), 4.35 (s, 2H), 6.28 (s, 1H), 6.56 (s, 2H), 7.07-7.09 (d, 2H), 7.20-7.32 (m, 4H), 7.43 (bs, 1H), 7.57- 7.59 (bs, 3H), 7.80-7.82 (m, 1H), 8.26- 8.30 (m, 2H). |
| 123 | 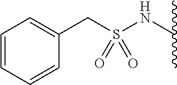 [SMILES: *NS(=O)(=O)Cc1ccccc1] | 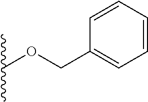 [SMILES: *OCc1ccccc1] | GS-2 | LC-MS: 590.7 [M + H]⁺; ¹HNMR (CD₃OD, 400 MHz): δ 4.05 (s, 2H), 4.34 (s, 2H), 5.20 (s, 2H), 6.23 (s, 1H), 6.55 (s, 2H), 7.00-7.01 (d, 2H), 7.09-7.11 (m, 4H), 7.17-7.24 (m, 4H), 7.29-7.31 (d, 1H), 7.45 (s, 1H), 7.57-7.59 (m, 2H), 7.61 (s, 1H), 7.82-7.83 (m, 1H), 8.4 (bs, |
| | | | | 1H), 8.28-8.30 (bs, 1H). |
| 124 | 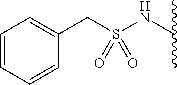 [SMILES: *NS(=O)(=O)Cc1ccccc1] | 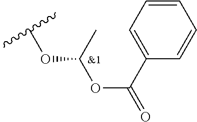 [SMILES: *O[C@H](C)OC(=O)c1ccccc1] | GS-2 | LC-MS: 649.3 [M + H]⁺; ¹HNMR (CD₃OD, 400 MHz): δ 1.54-1.55 (d, 3H), 4.15-4.16 (d, 2H), 4.35 (s, 2H), 6.23 (s, 1H), 6.55 (s, 2H), 7.08-7.09 (m, 2H), 7.16-7.25 (m, 4H), 7.30-7.32 (d, 1H), 7.35-7.39 (m, 2H), 7.42-7.45 (d, 1H), 7.48-7.57 (m, 3H), 7.67-7.73 (m, 4H), 8.19-8.21 (d, 1H), 8.30-8.32 (d, 1H). |
| 125 | 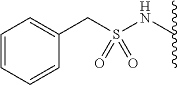 [SMILES: *NS(=O)(=O)Cc1ccccc1] | 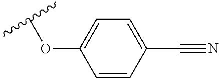 [SMILES: *Oc1ccc(C#N)cc1] | GS-2 | LC-MS: 602.3 [M + H]⁺; ¹HNMR (CD₃OD, 400 MHz): δ 4.19 (s, 2H), 4.39 (s, 2H), 6.40 (s, 1H), 6.58 (s, 2H), 7.06- 7.08 (d, 2H), 7.16-7.20 (m, 2H), 7.23- 7.29 (m, 3H), 7.36-7.38 (d, 1H), 7.43- 7.44 (d, 1H), 7.54-7.56 (m, 2H), 7.68- |
| | | | | 7.70 (d, 2H), 7.79-7.81 (m, 1H), 7.87 (s, |
| | | | | 1H), 8.21-8.23 (m, 1H), 8.37-8.39 (d, |
| | | | | 1H). |
| 126 | 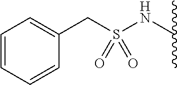 [SMILES: *NS(=O)(=O)Cc1ccccc1] | 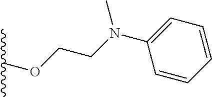 [SMILES: *OCCN(C)c1ccccc1] | GS-2 | LC-MS: 634.2 [M + H]⁺; ¹HNMR (CD₃OD, 400 MHz): δ 2.89 (s, 3H), 3.58- 3.61 (t, 2H), 4.10 (s, 2H), 4.21-4.23 (m, 2H), 4.31-4.33 (d, 2H), 4.34 (s, 2H), 6.26 (s, 1H), 6.51 (s, 2H), 6.72-6.78 (m, 3H), 7.03-7.05 (d, 2H), 7.13-7.23 (m, 5H), 7.30-7.32 (d, 1H), 7.40 (s, 1H), 7.43 (s, |
| | | | | 1H), 7.56-7.81 (m, 1H), 8.21-8.23 (m, |
| | | | | 1H), 8.27-8.29 (d, 1H). |
| 127 | 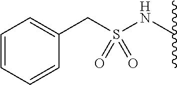 [SMILES: *NS(=O)(=O)Cc1ccccc1] | 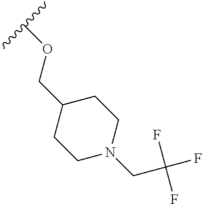 [SMILES: *OCC1CCN(CC(F)(F)F)CC1] | GS-3 | LC-MS: 680 [M + H]⁺. |
| 128 | 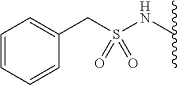 [SMILES: *NS(=O)(=O)Cc1ccccc1] | 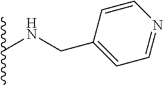 [SMILES: *NCc1ccncc1] | GS-3 | LC-MS: 591 [M + H]⁺. |
| 129 | 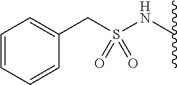 [SMILES: *NS(=O)(=O)Cc1ccccc1] | 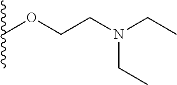 [SMILES: *OCCN(CC)CC] | GS-3 | LC-MS: 625 [M + H]⁺. |
| 130 | 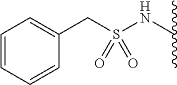 [SMILES: *NS(=O)(=O)Cc1ccccc1] | 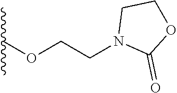 [SMILES: *OCCN1CCOC1=O] | GS-3 | LC-MS: 614 [M + H]⁺. |
| 131 | 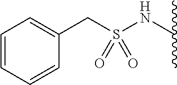 [SMILES: *NS(=O)(=O)Cc1ccccc1] | 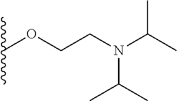 [SMILES: *OCCN(C(C)C)C(C)C] | GS-3 | LC-MS: 628 [M + H]⁺. |
| 132 | 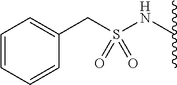 [SMILES: *NS(=O)(=O)Cc1ccccc1] | 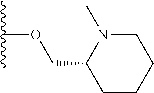 [SMILES: *OC[C@H]1CCCCN1C] | GS-3 | LC-MS: 612 [M + H]⁺. |
| 133 | 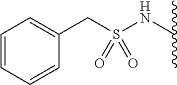 [SMILES: *NS(=O)(=O)Cc1ccccc1] | 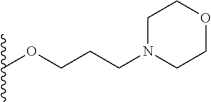 [SMILES: *OCCCN1CCOCC1] | GS-3 | LC-MS: 628 [M + H]⁺. |
| 134 | 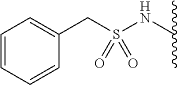 [SMILES: *NS(=O)(=O)Cc1ccccc1] | 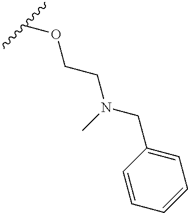 [SMILES: *OCCN(C)Cc1ccccc1] | GS-3 | LC-MS: 648 [M + H]⁺. |
| 135 | 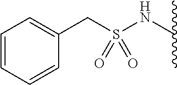 [SMILES: *NS(=O)(=O)Cc1ccccc1] | 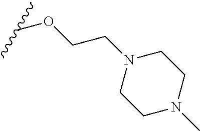 [SMILES: *OCCN1CCN(C)CC1] | GS-3 | LC-MS: 627 [M + H]⁺. |
| 136 | 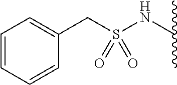 [SMILES: *NS(=O)(=O)Cc1ccccc1] | 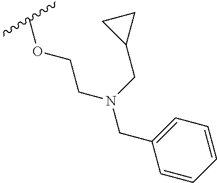 [SMILES: *OCCN(Cc1ccccc1)CC1CC1] | GS-3 | LC-MS: 688 [M + H]⁺. |
| 137 | 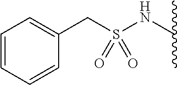 [SMILES: *NS(=O)(=O)Cc1ccccc1] | 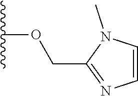 [SMILES: *OCc1nccn1C] | GS-3 | LC-MS: 595 [M + H]⁺. |
| 138 | 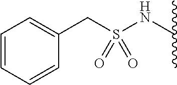 [SMILES: *NS(=O)(=O)Cc1ccccc1] | 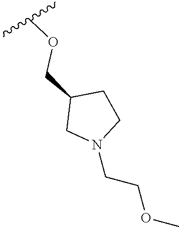 [SMILES: *OC[C@H]1CCN(CCOC)C1] | GS-3 | LC-MS: 642 [M + H]⁺. |
| 139 | 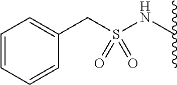 [SMILES: *NS(=O)(=O)Cc1ccccc1] | 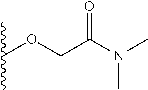 [SMILES: *OCC(=O)N(C)C] | GS-3 | LC-MS: 586 [M + H]⁺. |
| 140 | 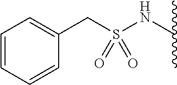 [SMILES: *NS(=O)(=O)Cc1ccccc1] | 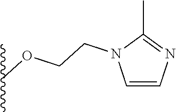 [SMILES: *OCCn1ccnc1C] | GS-3 | LC-MS: 609 [M + H]⁺. |
| 141 | 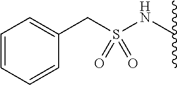 [SMILES: *NS(=O)(=O)Cc1ccccc1] | 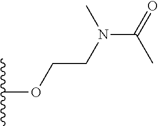 [SMILES: *OCCN(C)C(C)=O] | GS-3 | LC-MS: 600 [M + H]⁺. |
| 142 | 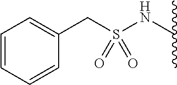 [SMILES: *NS(=O)(=O)Cc1ccccc1] | 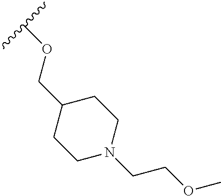 [SMILES: *OCC1CCN(CCOC)CC1] | GS-3 | LC-MS: 600 [M + H]⁺. |
| 143 | 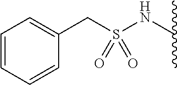 [SMILES: *NS(=O)(=O)Cc1ccccc1] | 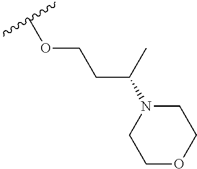 [SMILES: *OCC[C@H](C)N1CCOCC1] | GS-3 | LC-MS: 642 [M + H]⁺. |
| 144 | 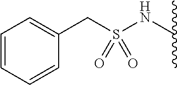 [SMILES: *NS(=O)(=O)Cc1ccccc1] | 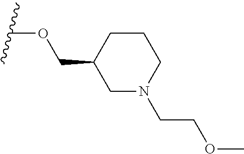 [SMILES: *OC[C@H]1CCCN(CCOC)C1] | GS-3 | LC-MS: 656 [M + H]⁺. |
| 145 | 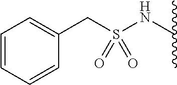 [SMILES: *NS(=O)(=O)Cc1ccccc1] | 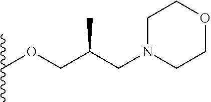 [SMILES: *OC[C@@H](C)CN1CCOCC1] | GS-3 | LC-MS: 642 [M + H]⁺. |
| 146 | 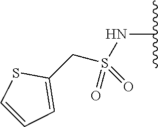 [SMILES: *NS(=O)(=O)Cc1cccs1] | 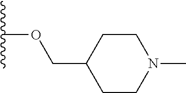 [SMILES: *OCC1CCN(C)CC1] | GS-2 | LC-MS: 618.4 [M + H]⁺; ¹HNMR (CD₃OD, 400 MHz): δ 1.28-1.47 (m, 3H), 1.72-1.76 (d, 3H), 2.65-2.72 (m, 2H), 2.78-2.83 (m, 2H), 3.35-3.38 (d, 2H), 4.09-4.10 (d, 2H), 4.36 (s, 2H), 4.42 (s, 2H), 6.23 (s, 1H), 6.55 (s, 2H), 6.84-6.85 (m, 1H), 6.89-6.91 (m, 1H), 7.32-7.34 (m, 2H), 7.49 (s, 1H), 7.59-7.63 (m, 3H), |
| | | | | 7.83-7.85 (m, 1H), 8.25-8.27 (m, 1H), |
| | | | | 8.30-8.32 (d, 1H). |
| 147 | 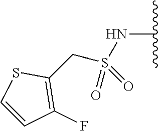 [SMILES: *NS(=O)(=O)Cc1sccc1F] | 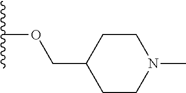 [SMILES: *OCC1CCN(C)CC1] | GS-2 | LC-MS: 634.2 [M + H]⁺; ¹HNMR (CD₃OD, 400 MHz): δ 1.29-1.40 (m, 4H), 1.74-1.77 (d, 2H), 2.68-2.74 (t, 2H), 2.79 (s, 3H), 3.36-3.39 (d, 2H) 4.10-4.11 (d, 2H), 4.34-4.36 (d, 3H), 6.22 (s, 1H), 6.55 (s, 2H), 6.72-6.74 (d, 2H), 7.30-7.33 (m, 2H), 7.47 (s, 1H), 7.56-7.6 (m, 2H), 7.82- 7.84 (d, 1H), 8.23-8.25 (d, 1H), 8.29-8.32 (d, 1H). |
| 148 | 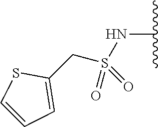 [SMILES: *NS(=O)(=O)Cc1cccs1] | 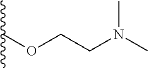 [SMILES: *OCCN(C)C] | GS-2 | LC-MS: 578.1 [M + H]⁺; ¹HNMR (CD₃OD, 400 MHz): δ 2.91 (s, 6H), 3.46- 3.48 (m, 2H), 4.36 (s, 2H), 4.40 (s, 2H), 4.55-4.57 (m, 2H), 6.31 (s, 1H), 6.58 (s, 2H), 6.87-6.89 (d, 2H), 6.90-6.92 (m, 1H), 7.33-7.36 (m, 2H), 7.43 (s, 1H), 7.58-7.61 (m, 2H), 7.74(s, 1H), 8.27-8.30 (d, 1H), 8.33-8.34 (d, 1H). |
| 149 | 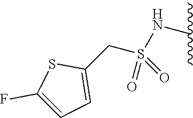 [SMILES: *NS(=O)(=O)Cc1ccc(F)s1] | 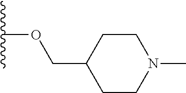 [SMILES: *OCC1CCN(C)CC1] | GS-2 | LC-MS: 634.1 [M + H]⁺; ¹HNMR (CD₃OD, 400 MHz): δ 135-1.45 (m, 2H), 1.81 (s, 3H), 2.73-2.80 (m, 5H), 3.39- 3.42 (d, 2H), 4.10-4.12 (d, 2H), 4.30 (s, 2H), 4.36 (s, 2H), 6.22 (s, 2H), 6.33-6.35 (m, 1H), 6.46-6.48 (t, 1H), 6.55 (s, 1H), 7.32-7.34 (d, 1H), 7.50 (s, 1H), 7.59-7.61 (m, 3H), 7.84-7.87 (m, 1H), 8.24-8.26 (t, |
| | | | | 1H), 8.29-8.31 (d, 1H). |
| 150 | 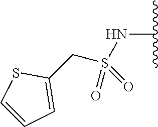 [SMILES: *NS(=O)(=O)Cc1cccs1] | 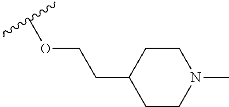 [SMILES: *OCCC1CCN(C)CC1] | GS-2 | LC-MS: 632.2 [M + H]⁺; ¹HNMR (CD₃OD, 400 MHz): δ 1.16-1.18 (d, 3H), 1.42-1.44 (d, 2H), 1.60-1.62 (d, 2H), 2.52-2.54 (t, 2H), 2.75 (s, 3H), 2.83 (s, 1H), 3.08-3.10 (m, 1H), 4.24-4.28 (t, 2H), 4.35 (s, 2H), 4.44 (s, 2H), 6.19 (s, 1H), 6.56 (s, 2H), 6.85-6.90 (m, 2H), 7.30-7.34 (d, 2H), 7.49 (s, 1H), 7.60-7.62 |
| | | | | (t, 3H), 7.86-7.88 (m, 1H), 8.28-8.30 (d, |
| | | | | 2H). |
| 151 | 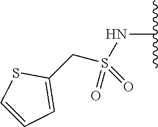 [SMILES: *NS(=O)(=O)Cc1cccs1] | 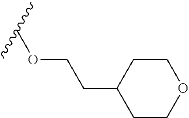 [SMILES: *OCCC1CCOCC1] | GS-2 | LC-MS: 620.1 [M + H]⁺; ¹HNMR (CD₃OD, 400 MHz): δ 3.10-3.20 (m, 4H), 3.40-3.50 (m, 4H), 3.60-4.00 (m, 4H),.36-4.39 (d, 2H), 4.57 (s, 2H), 6.30 (s, 1H), 6.58 (s, 2H), 6.87 (s, 1H), 6.92- 6.93 (t, 1H), 7.33-7.35 (d, 2H), 7.43 (s, 1H), 7.58-7.60 (d, 2H), 7.74 (s, 1H), 7.83-7.85 (d, 1H), 8.26-8.27 (d, 1H), |
| | | | | 8.32-8.33 (d, 1H). |
| 152 | 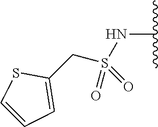 [SMILES: *NS(=O)(=O)Cc1cccs1] | 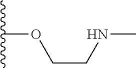 [SMILES: *OCCNC] | GS-2 | LC-MS: 564.2 [M + H]⁺; ¹HNMR (CD₃OD, 400 MHz): δ 2.72 (s, 3H), 3.31 (s, 2H), 4.36 (s, 2H), 4.40 (s, 2H), 4.48- 4.50 (t, 2H), 6.30 (s, 1H), 6.57 (s, 2H), 6.85-6.91 (m, 2H), 7.34-7.36 (t, 2H), 7.44 (s, 1H), 7.58-7.60 (m, 2H), 7.76 (s, 1H), 7.82-7.84 (m, 1H), 8.19-8.21 (d, 1H), 8.33-8.35 (d, 1H). |
| 153 | 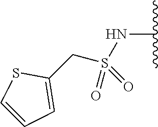 [SMILES: *NS(=O)(=O)Cc1cccs1] | 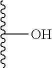 [SMILES: *O] | GS-2 | LC-MS: 507.1 [M + H]⁺; ¹HNMR (CD₃OD, 400 MHz): δ 4.31 (s, 2H),4.42 (s, 2H), 6.17 (s, 1H), 6.56 (s, 2H), 6.79 (s, 1H), 6.89-6.91 (t, 1H), 7.26-7.31 (m, 2H), 7.51-7.56 (m, 4H), 7.80 (s, 1H), 8.22-8.27 (m, 2H). |
| 154 | 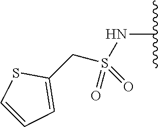 [SMILES: *NS(=O)(=O)Cc1cccs1] | 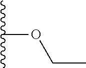 [SMILES: *OCC] | GS-2 | LC-MS: 535.3 [M + H]⁺; ¹HNMR (CD₃OD, 400 MHz): δ 1.11-1.18 (t, 3H), 4.17-4.22 (q, 2H), 4.33 (s, 2H), 4.40 (s, 2H), 6.19 (s, 1H), 6.55 (s, 2H), 6.80-6.81 (d, 1H), 6.87-6.90 (m, 1H), 7.29-7.32 (m, 2H), 7.52 (s, 1H), 7.55-7.58 (m, 3H), 7.81-7.83 (dd, 1H), 8.23-8.29 (m, 2H). |
| 155 | 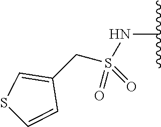 [SMILES: *NS(=O)(=O)Cc1ccsc1] | 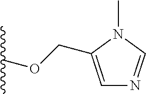 [SMILES: *OCc1cncn1C] | GS-2 | LC-MS: 600.9 [M + H]⁺; ¹HNMR (CD₃OD, 400 MHz): δ 3.64 (s, 3H), 4.20 (s, 2H), 4.34 (s, 2H), 5.33 (s, 2H), 6.13 (s, 1H), 6.48 (s, 2H), 6.83-6.85 (d, 1H), 7.01-7.03 (d, 1H), 7.23-7.26 (m, 1H), 7.32-7.34 (d, 1H), 7.38-7.40 (d, 2H), 7.54-7.58 (m, 2H), 7.65 (s, 1H), 7.79- 7.82 (m, 1H), 8.14-8.16 (m, 1H), 8.30- |
| | | | | 8.32 (d, 1H), 8.61 (s, 1H). |
| 156 | 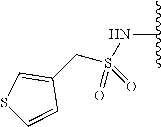 [SMILES: *NS(=O)(=O)Cc1ccsc1] | 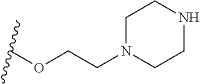 [SMILES: *OCCN1CCNCC1] | GS-2 | LC-MS: 619.1 [M + H]⁺; ¹HNMR (DMSO-d₆, 400 MHz): δ 3.47 (s, 8H), 4.21-4.26 (m, 2H), 4.37 (s, 4H), 4.51 (s, 2H), 6.19 (s, 1H), 6.45 (s, 2H), 6.86-6.88 (d, 1H), 7.17-7.19 (d, 1H), 7.40-7.45 (m, 2H), 7.49 (s, 1H), 7.56-7.59 (m, 2H), 7.86-7.88 (m, 2H), 8.23-8.26 (m, 1H), 8.33-8.35 (d, 1H), 8.49 (s, 1H), 9.77 (s, |
| | | | | 1H), 10.00-10.05 (m, 1H), 12.67-12.68 |
| | | | | (m, 1H). |
| 157 | 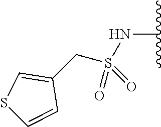 [SMILES: *NS(=O)(=O)Cc1ccsc1] | 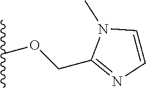 [SMILES: *OCc1nccn1C] | GS-2 | LC-MS: 601.3 [M + H]⁺; ¹HNMR (DMSO-d₆, 400 MHz): δ 3.76-3.77 (s, 3H), 4.32-4.33 (d, 2H), 4.39 (s, 2H), 5.54 (s, 2H), 6.21 (s, 1H), 6.47 (s, 2H), 6.89- 6.90 (d, 1H), 7.21-7.22 (d, 1H), 7.46-7.47 (m, 2H), 7.55 (s, 1H), 7.61-7.67 (m, 3H), 7.84 (s, 1H), 7.92-7.93 (d, 1H), 8.25-8.26 (d, 1H), 8.41-8.43 (m, 4H), 9.78 (s, 1H). |
| 158 | 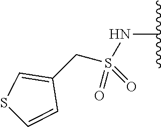 [SMILES: *NS(=O)(=O)Cc1ccsc1] | 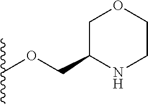 [SMILES: *OC[C@H]1COCCN1] | GS-2 | LC-MS: 606.3 [M + H]⁺; ¹HNMR (CD₃OD, 400 MHz): δ 3.17-3.24 (m, 1H), 3.31-3.34 (m, 1H), 3.55-3.58 (m, 1H), 3.61-3.65 (m, 1H), 3.69-3.76 (m, 1H), 3.92-3.97 (m, 2H), 4.22 (s, 2H), 4.34 (s, 2H), 4.37-4.45 (m, 2H), 6.26 (s, 1H), 6.53 (s, 2H), 6.85 (d, 1H), 7.03 (d, 1H), 7.26-7.28 (m, 1H), 7.33 (d, 1H), 7.39 (s, |
| | | | | 1H), 7.53-7.59 (m, 2H), 7.78-7.81 (m, |
| | | | | 2H), 8.22-8.24 (m, 1H), 8.28-8.32 (d, |
| | | | | 1H). |
| 159 | 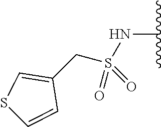 [SMILES: *NS(=O)(=O)Cc1ccsc1] | 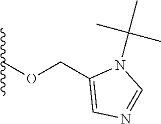 [SMILES: *OCc1cncn1C(C)(C)C] | GS-2 | LC-MS: 643.3 [M + H]⁺; ¹HNMR (CD₃OD, 400 MHz): δ 1.64 (s, 9H), 4.18 (s, 2H), 4.34 (s, 2H), 5.48 (s, 2H), 6.16 (s, 1H), 6.51 (s, 2H), 6.83-6.84 (d, 1H), 7.0 (d, 1H), 7.23-7.25 (q, 1H), 7.30-7.32 (d, 1H), 7.37-7.39 (d, 2H), 7.54-7.59 (m, 3H), 7.78-7.81 (t, 1H), 8.15-8.17 (t, 1H), 8.29-8.3 (d, 1H), 8.72 (s, 1H). |
| 160 | 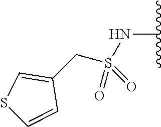 [SMILES: *NS(=O)(=O)Cc1ccsc1] | 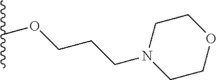 [SMILES: *OCCCN1CCOCC1] | GS-2 | LC-MS: 634.0 [M + H]⁺; ¹HNMR (CD₃OD, 400 MHz): δ 2.00-2.07 (m, 2H), 2.82-2.89 (m, 2H), 2.99-3.03 (t, 2H), 3.24-3.28 (d, 2H), 3.32-3.69 (t, 2H), 3.93-3.97 (m, 2H), 4.23 (s, 2H), 4.29- 4.32 (m, 4H), 6.21 (d, 1H), 6.54 (s, 2H), 6.83-6.86 (d, 1H), 7.04-7.06 (d, 1H), 7.28-7.33 (m, 2H), 7.39-7.43 (d, 1H), |
| | | | | 7.55-7.61 (m, 2H), 7.64 (s, 1H), 7.80- |
| | | | | 7.83 (m, 1H), 8.24-8.26 (d, 1H), 8.29- |
| | | | | 8.31 (d, 1H). |
| 161 | 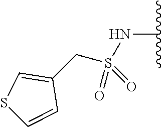 [SMILES: *NS(=O)(=O)Cc1ccsc1] | 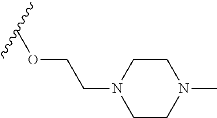 [SMILES: *OCCN1CCN(C)CC1] | GS-2 | LC-MS: 633.4 [M + H]⁺; ¹HNMR (CD₃OD, 400 MHz): δ 2.91 (s, 3H), 3.34- 3.55 (m, 10H), 4.25 (s, 2H), 4.33 (s, 2H), 4.53-4.55 (m, 2H), 6.25 (s, 1H), 6.55 (s, 2H), 6.87-6.88 (d, 1H), 7.07-7.08 (d, 1H), 4.28-7.32 (m, 2H), 7.40 (s, 1H), 7.54-7.60 (m, 2H), 7.77 (s, 1H), 7.80- 7.82 (m, 1H), 8.24-8.26 (m, 1H), 8.30- |
| | | | | 8.32 (d, 1H). |
| 162 | 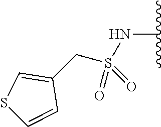 [SMILES: *NS(=O)(=O)Cc1ccsc1] | 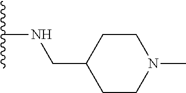 [SMILES: *NCC1CCN(C)CC1] | GS-2 | LC-MS: 631.0 [M + H]⁺; ¹HNMR (CD₃OD, 40 MHz): δ 1.17-1.20 (m, 3H), 1.36-1.37 (m, 2H), 1.79-1.82 (d, 2H), 2.36-2.42 (t, 2H), 2.74 (s, 3H), 3.23 (s, 2H), 4.25 (s, 2H), 4.33 (s, 2H), 6.22 (s, 1H), 6.51 (s, 2H), 6.84-6.86 (d, 2H), 7.02 (s, 1H), 7.24-7.32 (s, 3H), 7.40 (s, 1H), 7.54-7.56 (m, 3H), 7.79-7.82 (m, 1H), |
| | | | | 8.19-8.23 (m, 2H). |
| 163 | 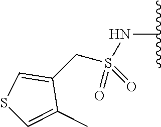 [SMILES: *NS(=O)(=O)Cc1cscc1C] | 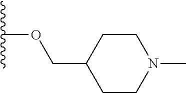 [SMILES: *OCC1CCN(C)CC1] | GS-2 | LC-MS: 632.4 [M + H]⁺; ¹HNMR (CD₃OD, 400 MHz): δ 1.36-1.39 (d, 2H), 1.75-1.79 (d, 3H), 2.04 (s, 3H), 2.69-2.77 (t, 5H), 3.36-3.45 (t, 2H), 4.08-4.10 (d, 2H), 4.19 (s, 2H), 4.39 (s, 2H), 6.24 (s, 1H), 6.52 (s, 2H), 6.92-6.93 (d, 2H), 7.30-7.32 (d, 1H), 7.39 (s, 1H), 7.55-7.57 (m, 2H), 7.78-7.81 (m, 1H), 8.20-8.23 (d, |
| | | | | 1H), 8.27-8.29 (d, 1H). |
| 164 | 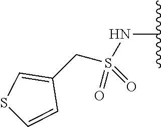 [SMILES: *NS(=O)(=O)Cc1ccsc1] | 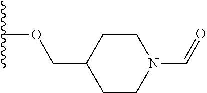 [SMILES: *OCC1CCN(C=O)CC1] | GS-2 | LC-MS: 632.3 [M + H]⁺; ¹HNMR (CD₃OD, 400 MHz): δ 0.7-1.15 (m, 3H), 1.42-1.64 (m, 2H), 1.72 (bs, 1H), 2.22- 2.47 (m, 1H), 2.73-2.91 (m, 1H), 3.45- 3.68 (m, 1H), 4.05-4.08 (m, 3H), 4.24 (s, 2H), 4.35 (s, 1H), 6.22 (s, 2H), 6.54 (s, 2H), 6.88-6.90 (d, 1H), 7.05-7.07 (d, 1H), 7.29-7.33 (m, 2H), 7.48 (s, 1H), |
| | | | | 7.59-7.62 (t, 2H), 7.84-7.86 (d, 2H), |
| | | | | 8.20-8.25 (m, 1H), 8.31-8.33 (d, 1H). |
| 165 | 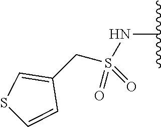 [SMILES: *NS(=O)(=O)Cc1ccsc1] | 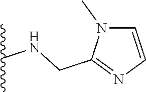 [SMILES: *NCc1nccn1C] | GS-2 | LC-MS: 600.3 [M + H]⁺; ¹HNMR (CD₃OD, 400 MHz): δ 3.69 (s, 3H), 4.19 (s, 2H), 4.33 (s, 2H), 4.65 (s, 2H), 6.27 (s, 1H), 6.49 (s, 2H), 6.83-6.84 (d, 1H), 6.99-7.0 (m, 1H), 7.21-7.25 (m, 2H), 7.27-7.30 (m, 2H), 7.36 (s, 1H), 7.38 (s, 1H), 7.50-7.54 (m, 2H), 7.76-7.79 (m, 1H), 8.14-8.16 (m, 1H), 8.23-8.25 (d, 1H). |
| 166 | 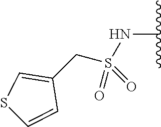 [SMILES: *NS(=O)(=O)Cc1ccsc1] | 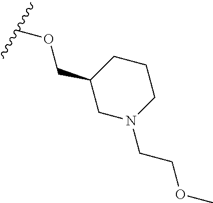 [SMILES: *OC[C@H]1CCCN(CCOC)C1] | GS-2 | LC-MS: 663.2 [M − H]⁻; ¹HNMR (CD₃OD, 400 MHz): δ 1.65-1.80 (m, 4H), 2.12-2.36 (m, 2H), 2.46-2.51 (dd, 2H), 3.36 (s, 3H), 3.61 (s, 3H), 4.16-4.23 (m, 4H), 4.36 (s, 2H), 6.24 (s, 1H), 6.56 (s, 2H), 6.82-6.84 (d, 1H), 7.05 (s, 1H), 7.29-7.33 (m, 1H), 7.35-7.37 (d, 1H), 7.42 (s, 1H), 7.57-7.63 (m, 2H), 7.65 (s, 1H), 7.83-7.85 (m, 2H), 8.26-8.28 (d, 2H), 8.32-8.34 (d, 1H). |
| 167 | 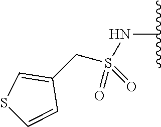 [SMILES: *NS(=O)(=O)Cc1ccsc1] | 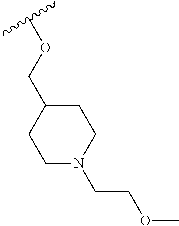 [SMILES: *OCC1CCN(CCOC)CC1] | GS-2 | LC-MS: 661.9 [M + H]⁺; ¹HNMR (DMSO-d₆, 400 MHz): δ 2.62-2.72 (m, 3H), 3.15-3.17 (d, 2H), 3.28 (s, 3H), 3.34-3.36 (d, 2H), 3.62 (s, 1H), 3.68-3.71 (t, 2H), 3.98-3.99 (d, 2H), 4.25-4.27 (d, 2H), 4.35 (s, 2H), 6.17 (s, 1H), 6.43 (s, 2H), 6.84-6.86 (d, 2H), 7.15 (s, 2H), 7.40-7.44 (m, 2H), 7.50 (s, 2H), 7.56- 7.61 (m, 2H), 7.67(s, 1H), 7.88-7.91 (t, 2H), 8.25-8.29 (t, 1H), 8.33-8.34 (d, 1H), 9.79 (s, 1H), 10.43 (bs, 1H). |
| 168 | 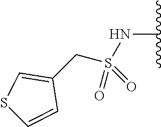 [SMILES: *NS(=O)(=O)Cc1ccsc1] | 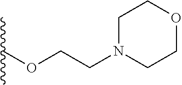 [SMILES: *OCCN1CCOCC1] | GS-2 | LC-MS: 620.1 [M + H]⁺; ¹HNMR (CD₃OD, 400 MHz): δ 3.13-3.15 (m, 4H), 3.50-3.56 (m, 2H), 3.80-3.90 (m, 4H), 4.24 (s, 2H), 4.36 (s, 2H), 4.57-4.60 (t, 2H), 6.30 (s, 1H), 6.57 (s, 2H), 6.88-6.90 (d, 1H), 7.10-7.10 (m, 1H), 7.31-7.36 (m, 2H), 7.40 (s, 1H), 7.56-7.62 (m, 2H), 7.75 (s, 1H), 7.82-7.84 (m, 1H), 8.25- |
| | | | | 8.28 (d, 1H), 8.32-8.34 (d, 1H). |
| 169 | 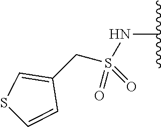 [SMILES: *NS(=O)(=O)Cc1ccsc1] | 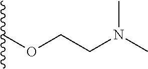 [SMILES: *OCCN(C)C] | GS-2 | LC-MS: 578.3 [M + H]⁺; ¹HNMR (CD₃OD, 300 MHz): δ 2.91 (s, 6H), 3.40-3.50 (t, 2H), 4.24 (s, 2H), 4.36 (s, 2H), 4.56-4.57 (t,2H), 6.30 (s, 1H), 6.57 (s, 2H), 6.89-6.91 (d, 1H), 7.09 (s, 1H), 7.30-7.41 (m, 3H), 7.58-7.59 (m, 2H), 7.74 (s, 1H), 7.82-7.84 (m, 1H), 8.25- 8.34 (m, 2H). |
| 170 | 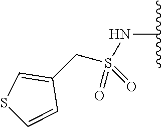 [SMILES: *NS(=O)(=O)Cc1ccsc1] | 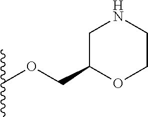 [SMILES: *OC[C@H]1CNCCO1] | GS-2 | LC-MS: 606.1 [M + H]⁺; ¹HNMR (CD₃OD, 400 MHz): δ 2.81-2.88 (m, 2H), 3.07-3.10 (d, 1H), 3.18-3.22 (d, 1H), 3.45-3.56 (m, 1H), 3.81-3.87 (m, 2H), 4.21-4.22 (m, 2H), 4.29-4.27 (m, 2H), 4.30-4.33 (m, 2H), 6.18 (s, 1H), 6.53 (s, 2H), 6.82-6.84 (d, 1H), 7.00-7.02 (d, 1H), 7.26-7.28 (m, 1H), |
| | | | | 7.28-7.33 (d, 1H),7.44 (s, 1H), 7.54-7.80 |
| | | | | (m, 2H), 7.63 (s, 1H), 7.80-7.83 (m, 1H), |
| | | | | 8.21-8.23 (m, 1H), 8.28-8.32 (d, 1H). |
| 171 | 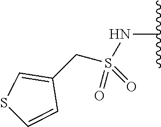 [SMILES: *NS(=O)(=O)Cc1ccsc1] | 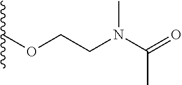 [SMILES: *OCCN(C)C(C)=O] | GS-2 | LC-MS: 605.9 [M + H]⁺; ¹HNMR (CD₃OD, 400 MHz): δ 1.89 (s, 2H), 2.04 (s, 1H), 2.85 (s, 1H), 2.97 (s, 3H), 3.58- 3.65 (m, 2H), 4.22 (s, 2H), 4.31-4.33 (m, 3H), 4.37-4.39 (m, 1H), 6.19-6.21 (d, 1H), 6.52-6.54 (d, 2H), 6.84-6.87 (t, 1H), 6.97 (s, 1H), 7.24-7.31 (m, 2H), 7.45 (s, 1H), 7.52-7.58 (m, 2H), 7.79-7.81 (m, |
| | | | | 1H), 8.21-8.31 (m, 2H). |
| 172 | 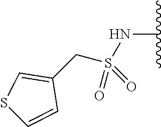 [SMILES: *NS(=O)(=O)Cc1ccsc1] | 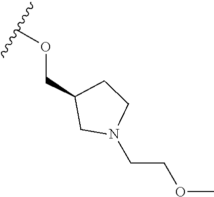 [SMILES: *OC[C@H]1CCN(CCOC)C1] | GS-2 | LC-MS: 646.1 [M − H]⁻; ¹HNMR (CD₃OD, 400 MHz): δ 0.77 (s, 2H), 1.28 (s, 2H), 1.93 (bs, 2H), 2.83 (s, 5H), 3.36 (s, 3H), 3.57 (s, 3H), 4.35 (s, 2H), 4.44- 4.46 (d, 2H), 6.22 (bs, 2H), 6.71-6.90 (m, 2H), 6.99 (s, 1H), 7.07-7.09 (d, 2H), 7.20 (s, 1H), 7.44-7.46 (d, 2H), 7.77-7.79 (d, 1H), 7.82-7.83 (m, 1H), 7.92-7.98 (m, 1H). |
| 173 | 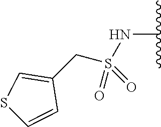 [SMILES: *NS(=O)(=O)Cc1ccsc1] | 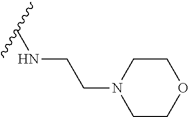 [SMILES: *NCCN1CCOCC1] | GS-2 | LC-MS: 619.0 [M + H]⁺; ¹HNMR (CD₃OD, 400 MHz): δ 3.0 (t, 2H), 3.20- 3.23 (t, 2H), 3.33-3.36 (m, 2H), 3.48 (t, 2H), 3.64-3.67 (t, 2H), 3.80 (d, 2H), 4.37 (s, 2H), 4.44 (s, 2H), 6.35 (s, 1H), 6.59 (s, 2H), 6.87-6.89 (m, 2H), 7.3 (m, 2H), 7.43 (m, 2H), 7.57-7.59 (m, 2H), 7.81 (m, 1H), 8.27 (m, 1H), 8.29(d, 1H). |
| 174 | 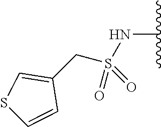 [SMILES: *NS(=O)(=O)Cc1ccsc1] | 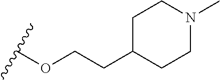 [SMILES: *OCCC1CCN(C)CC1] | GS-2 | LC-MS: 632.0 [M + H]⁺; ¹HNMR (CD₃OD, 400 MHz): δ 1.29-1.33 (m, 2H), 1.47 (s, 3H), 1.60-1.68 (d, 2H), 2.58 (s, 2H), 2.78-2.82 (d, 3H), 4.26-4.28 (d, 4H), 4.36 (s, 2H), 4.88-4.92 (d, 2H), 6.18 (s, 1H), 6.56 (s, 2H), 6.87-6.93 (d, 1H), 7.05-7.07 (d, 1H), 7.29-7.30 (m, 1H), 7.30-7.34 (d, 1H), 7.45 (s, 1H), 7.60-7.62 |
| | | | | (t, 3H), 7.84-7.86 (t, 1H), 8.26-8.28 (d, |
| | | | | 1H), 8.30-8.32 (d, 1H). |
| 175 | 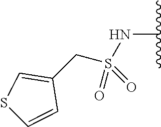 [SMILES: *NS(=O)(=O)Cc1ccsc1] | 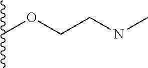 [SMILES: *OCCNC] | GS-2 | LC-MS: 564.3 [M + H]⁺; ¹HNMR (CD₃OD, 400 MHz): δ 2.70-2.74(t, 3H), 3.30-3.34(d, 2H), 4.24(s, 2H), 4.36(s, 2H), 4.44-4.48(t, 2H), 6.28(s, 1H), 6.56(s, 2H), 6.86-6.89(d, 1H), 7.00- 7.08(d, 1H), 7.28-7.30(m, 1H), 7.32- 7.36(d, 1H), 7.42(s, 1H), 7.58-7.62 (m, 2H), 7.73 (s, 1H), 7.80-7.84 (t, 1H), 8.24- |
| | | | | 7.26 (d, 1H), 8.30-8.32 (d, 1H). |
| 176 | 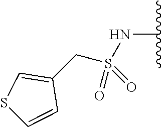 [SMILES: *NS(=O)(=O)Cc1ccsc1] | 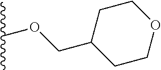 [SMILES: *OCC1CCOCC1] | GS-2 | LC-MS: 604.9 [M + H]⁺; ¹HNMR (DMSO-d₆, 400 MHz): δ 1.05-1.09 (m, 2H), 1.24-1.29 (m, 1H), 1.52-1.56 (m, 1H), 2.93-2.96 (t, 2H), 3.64-3.66 (d, 2H), 3.93-3.95 (d, 2H), 4.24-4.26 (d, 2H), 4.34 (s, 2H), 6.18 (s, 1H), 6.44 (s, 2H), 6.84- 6.85 (d, 1H), 7.16-7.17 (d, 1H), 7.39-7.44 (m, 2H), 7.51 (s, 1H), 7.58-7.60 (m, 2H), |
| | | | | 7.86-7.89 (m, 1H), 8.22-8.26 (m, 1H), |
| | | | | 8.34-8.36 (d, 1H), 8.42 (bs, 1H), 9.78 (s, |
| | | | | 1H). |
| 177 | 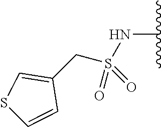 [SMILES: *NS(=O)(=O)Cc1ccsc1] | 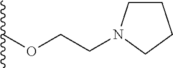 [SMILES: *OCCN1CCCC1] | GS-2 | LC-MS: 604.3 [M + H]⁺; ¹HNMR (CD₃OD, 300 MHz): δ 1.28-1.38 (m, 4H), 2.20 (s, 4H), 3.47 (s, 2H), 4.23 (s, 2H), 4.36 (s, 2H), 4.52-4.56 (m, 2H), 6.29 (s, 1H), 6.57 (s, 2H), 6.90-6.92 (d, 1H), 7.09 (s, 1H), 7.29-7.35 (m, 2H), 7.41 (s, 1H), 7.57-7.60 (m, 2H), 7.74 (s, 1H), 7.83- 7.85 (d, 1H), 8.26-8.28 (d, 1H), 8.33-8.35 |
| | | | | (d, 1H). |
| 178 | 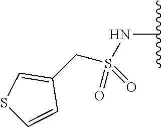 [SMILES: *NS(=O)(=O)Cc1ccsc1] | 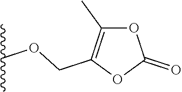 [SMILES: *OCc1oc(=O)oc1C] | GS-2 | LC-MS: 619.0 [M + H]⁺; ¹HNMR (CD₃OD, 400 MHz): δ 2.67 (s, 3H), 4.25 (s, 2H), 4.34 (s, 2H), 4.99 (s, 2H), 6.14 (s, 1H), 6.50 (s, 2H), 6.86-6.87 (d, 1H), 7.04-7.05 (m, 1H), 7.26-7.31 (m, 2H), 7.48 (m, 1H), 7.54-7.57 (m, 2H), 7.62 (s, 1H), 7.80-7.82 (m, 1H), 8.18-8.20 (m, 1H), 8.28-8.30 (d, 1H). |
| 179 | 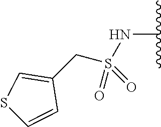 [SMILES: *NS(=O)(=O)Cc1ccsc1] | 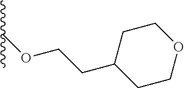 [SMILES: *OCCC1CCOCC1] | GS-2 | LC-MS: 618.9 [M + H]⁺; ¹HNMR (CD₃OD, 400 MHz): δ 1.03-1.12 (m, 3H), 1.29-1.43 (m, 6H), 3.11 (t, 2H), 3.73- 3.78 (dd, 2H), 4.22-4.25 (m, 3H), 4.35 (s, 2H), 6.20 (s, 1H), 6.55 (s, 2H), 6.86-6.87 (d, 1H), 7.02-7.03 (d, 1H), 7.28-7.33 (m, 2H), 7.49 (s, 1H), 7.56-7.61 (m, 2H), 7.82-7.85 (m, 1H), 8.24-8.26 (m, 1H), |
| | | | | 8.30-8.32 (d, 1H). |
| 180 | 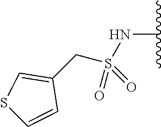 [SMILES: *NS(=O)(=O)Cc1ccsc1] | 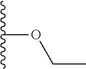 [SMILES: *OCC] | GS-2 | LC-MS: 535.0 [M + H]⁺; ¹HNMR (CD₃OD, 400 MHz): δ 1.12-1.16 (t, 3H), 4.19 (m, 2H), 4.24 (s, 2H), 4.34 (s, 2H), 6.18 (s, 1H), 6.54 (s, 2H), 6.85-6.86 (d, 1H), 7.01 (m, 1H), 7.27 (s, 1H), 7.29- 7.33 (d, 1H), 7.49 (s, 1H), 7.55-7.57 (m, 3H), 7.82 (m, 1H), 8.24 (m, 1H), 8.27- 8.30 (d, 1H). |
| 181 | 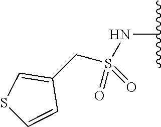 [SMILES: *NS(=O)(=O)Cc1ccsc1] | 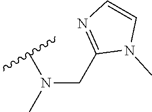 | GS-2 | LC-MS: 614.4 [M + H]⁺; ¹HNMR (CD₃OD, 400 MHz): δ 2.99 (s, 3H), 3.58 (s, 3H), 4.34 (s, 2H), 4.39 (s, 2H), 4.72 (s, 2H), 6.25 (s, 2H), 6.64 (s, 1H), 6.91- 7.00 (m, 2H), 7.14-7.15 (d, 1H), 7.28- 7.31 (m, 5H), 7.42-7.51 (m, 2H), 7.77- 7.79 (dd, 1H), 7.96-7.98 (m, 1H), 8.18- 8.20 (d, 1H). |
| 182 | 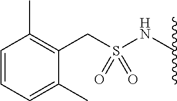 [SMILES: *NS(=O)(=O)Cc1c(C)cccc1C] | 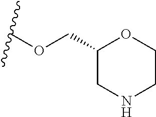 [SMILES: *OC[C@H]1CNCCO1] | GS-2 | LC-MS: 628.4 [M + H]⁺; ¹HNMR (CD₃OD, 400 MHz): δ 2.17 (s, 6H), 2.87- 2.93 (m, 3H), 3.11-3.14 (d, 2H), 3.20- 3.24 (d, 2H), 3.59-3.65 (m, 1H), 3.82- 3.92 (m, 2H), 4.20-4.33 (m, 2H), 4.37- 4.85 (d, 4H), 6.33 (d, 1H), 6.93-6.94 (d, 2H), 7.01-7.05 (t, 1H), 7.40 (d, 1H), 7.53-7.59 (m, 2H), 7.61 (s, 1H), 7.76- |
| | | | | 7.78 (m, 1H), 8.022-8.024 (d, 1H), 8.02- |
| | | | | 8.03 (d, 1H). |
| 183 | 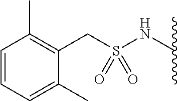 [SMILES: *NS(=O)(=O)Cc1c(C)cccc1C] | 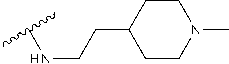 [SMILES: *NCCC1CCN(C)CC1] | GS-2 | LC-MS: 651.1 [M − H]⁻; ¹HNMR (CD₃OD, 400 MHz): δ 1.24-1.26 (m, 2H), 1.38-1.40 (m, 2H), 1.82-1.84 (m, 2H),2.17 (s, 6H), 2.40-2.50 (m, 3H), 2.67 (s, 3H), 3.24-3.27 (m, 4H), 4.36 (s, 2H), 4.41 (s, 2H), 6.44 (s, 1H), 6.57 (s, 2H), 6.93-6.95 (m,2H), 7.03-7.05 (m, 1H), |
| | | | | 7.26 (s, 1H), 7.30-7.32 (m, 1H), 7.380- |
| | | | | 7.384 (m, 1H), 7.55-7.58(m, 2H), 7.76- |
| | | | | 7.78 (m, 1H), 8.22-8.28 (m, 2H). |
| 184 | 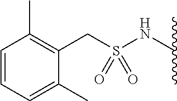 [SMILES: *NS(=O)(=O)Cc1c(C)cccc1C] | 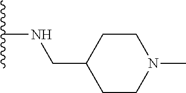 [SMILES: *NCC1CCN(C)CC1] | GS-2 | LC-MS: 639.4 [M + H]⁺; ¹HNMR (CD₃OD, 400 MHz): δ 1.22-1.40 (m, 3H), 1.71-1.75 (m, 4H), 2.20 (s, 6H), 2.75- 2.85 (s, 4H), 3.13-3.19 (m, 3H), 3.35- 3.45 (m, 2H), 4.39 (s, 2H), 6.52-6.59 (m, 3H), 6.94-7.07 (m,3H), 7.21 (s, 1H), 7.30-7.38 (m, 2H), 7.47-7.54 (m, 2H), |
| | | | | 7.73-7.75 (m, 1H), 8.14-8.24 (m, 2H). |
| 185 | 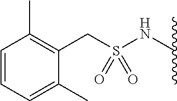 [SMILES: *NS(=O)(=O)Cc1c(C)cccc1C] | 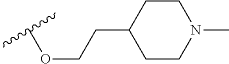 [SMILES: *OCCC1CCN(C)CC1] | GS-2 | LC-MS: 654.4 [M + H]⁺; ¹HNMR (CD₃OD, 400 MHz): 1.23-1.68 (m, 7H), 2.19 (s, 6H), 2.57-2.76 (m, 5H), 3.34- 3.38 (m, 2H), 4.25-4.27 (d, 2H), 4.36 (s, 2H), 4.42 (s, 2H), 6.38 (s, 1H), 6.55 (s, 2H), 6.95-6.97 (d, 2H), 7.04-7.08 (t, 1H), 7.32-7.34 (d, 1H), 7.40-7.406 (d, 1H), |
| | | | | 7.56-7.62 (m, 3H), 7.78-7.81 (m, 1H), |
| | | | | 8.25-8.27 (d, 1H), 8.30-8.32 (d, 1H). |
| 186 | 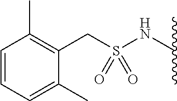 [SMILES: *NS(=O)(=O)Cc1c(C)cccc1C] | 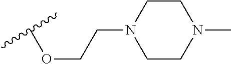 [SMILES: *OCCN1CCN(C)CC1] | GS-2 | LC-MS: 655.4 [M + H]⁺; ¹HNMR (CD₃OD, 400 MHz): δ 2.15 (s, 6H), 2.86 (s, 3H), 3.27-3.28 (t, 2H), 3.29-3.32 (t, 7H), 4.33 (s, 2H), 4.41 (s, 2H), 4.49-4.51 (d, 2H), 6.38 (s, 1H), 6.55 (s, 2H), 6.96- 6.98 (d, 2H), 7.04-7.06 (d, 1H), 7.30-7.32 (d, 2H), 7.43 (s, 1H), 7.56-7.58 (t, 2H), |
| | | | | 7.75 (s, 1H), 7.791-7.795 (d, 1H), 8.26- |
| | | | | 8.31 (m, 2H). |
| 187 | 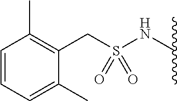 [SMILES: *NS(=O)(=O)Cc1c(C)cccc1C] | 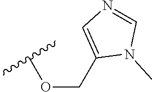 [SMILES: *OCc1cncn1C] | GS-2 | LC-MS: 623.4 [M + H]⁺; ¹HNMR (CD₃OD, 300 MHz): δ 2.17 (s, 6H), 3.62 (s, 3H), 4.37-4.39 (d, 4H), 5.32 (s, 2H), 6.31 (s, 1H), 6.49 (s, 2H), 6.92-6.94 (d, 2H), 7.00-7.05 (t, 1H), 7.32-7.37 (m, 3H), 7.55-7.58 (m, 2H), 7.60 (s, 1H), 7.76-7.79 (m, 1H), 8.15-8.18 (m, 1H), |
| | | | | 8.30-8.33 (d, 1H), 8.61(s, 1H). |
| 188 | 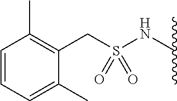 [SMILES: *NS(=O)(=O)Cc1c(C)cccc1C] | 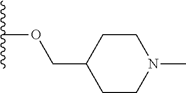 [SMILES: *OCC1CCN(C)CC1] | GS-2 | LC-MS: 640.4 [M + H]⁺; ¹HNMR (CD₃OD, 400 MHz): δ 1.29-1.47 (m, 2H), 1.74-1.81 (m, 3H), 2.19 (s, 6H), 2.68- 2.76 (m, 5H), 3.34-3.38 (m, 2H), 4.09- 4.11 (d, 2H), 4.36 (s, 2H), 4.41 (s, 2H), 6.38 (s, 1H), 6.55 (s, 2H), 6.95-6.97 (d, 2H), 7.04-7.08 (t, 1H), 7.32-7.34 (d, 1H), |
| | | | | 7.40-7.406 (d, 1H), 7.56-7.62 (m, 3H), |
| | | | | 7.78-7.81 (m, 1H), 8.25-8.27 (d, 1H), |
| | | | | 8.30-8.32 (d, 1H). |
| 189 | 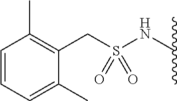 [SMILES: *NS(=O)(=O)Cc1c(C)cccc1C] | 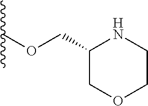 [SMILES: *OC[C@H]1COCCN1] | GS-2 | LC-MS: 628.4 [M + H]⁺; ¹HNMR (CD₃OD, 400 MHz): δ 2.15 (s, 6H), 3.17- 3.21 (m, 1H), 3.52-3.61 (m, 2H), 3.62- 3.71 (m, 1H), 3.87-3.94 (m, 3H), 4.30- 4.35 (m, 4H), 4.38-4.443 (m, 3H), 6.381- 6.386 (d, 1H), 6.54 (s, 2H), 6.92-6.94 (d, 2H), 7.32-7.34 (d, 1H), 7.40 (d, 1H), |
| | | | | 7.54-7.58 (m, 2H), 7.76-7.79 (m, 2H), |
| | | | | 8.23-8.25 (m, 1H), 8.30-8.32 (d, 1H). |
| 190 | 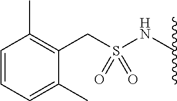 [SMILES: *NS(=O)(=O)Cc1c(C)cccc1C] | 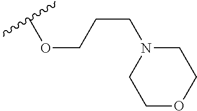 [SMILES: *OCCCN1CCOCC1] | GS-2 | LC-MS: 656.4 [M + H]⁺; ¹HNMR (CD₃OD, 400 MHz): δ 1.98-2.01 (m, 2H), 2.158 (s, 6H), 2.70-2.81 (m, 3H), 2.95- 2.99 (t, 2H), 3.17-3.20 (d, 2H), 3.59-3.65 (t, 2H) 3.89-3.92 (d, 2H), 4.3-4.33 (m, 4H), 4.39 (s, 2H), 6.38 (s, 1H), 6.55 (s, 1H), 6.95-6.97 (d, 2H), 7.03-7.07 (t, 1H), |
| | | | | 7.30-7.32 (d, 1H), 7.38 (s, 1H), 7.55-7.81 |
| | | | | (m, 3H), 7.79-7.81 (d, 1H), 8.26-8.30 (t, |
| | | | | 2H). |
| 191 | 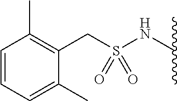 [SMILES: *NS(=O)(=O)Cc1c(C)cccc1C] | 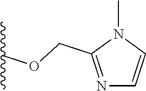 [SMILES: *OCc1nccn1C] | GS-2 | LC-MS: 623.4 [M + H]⁺; ¹HNMR (CD₃OD, 300 MHz): δ 2.15 (s, 6H), 3.73 (s, 3H), 4.37-4.38 (d, 4H), 5.48 (s, 2H), 6.33 (s, 1H), 6.50 (s, 2H), 6.90-6.93 (d, 2H), 6.99-7.04 (t, 1H), 7.34 (s, 2H), 7.37- 7.39 (d, 1H), 7.54-7.58 (m, 2H), 7.70 (s, 1H), 7.76-7.78 (m, 2H), 8.18-8.21 (m, |
| | | | | 1H), 8.32-8.35 (d, 1H). |
| 192 | 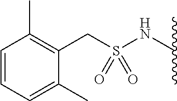 [SMILES: *NS(=O)(=O)Cc1c(C)cccc1C] | 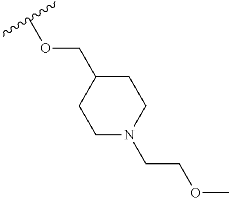 [SMILES: *OCC1CCN(CCOC)CC1] | GS-2 | LC-MS: 684.4 [M + H]⁺; ¹HNMR (CD₃OD, 400 MHz): δ 1.27-1.47 (m, 3H), 1.70-1.79 (m, 3H), 2.18 (s, 6H), 2.69- 2.80 (t, 2H), 3.17-3.2 (t, 2H), 3.37-3.41 (d, 4H), 3.55-3.65 (t, 2H), 4.07-4.08 (d, 2H), 4.35-4.39 (d, 4H), 6.35 (s, 1H), 6.53 (s, 2H), 6.93-6.98 (t, 2H), 7.02-7.06 (t, 1H), 7.30-7.33 (m, 1H), 7.37 (s, 1H), 7.54-7.59 (m, 3H), 7.76-7.78 (m, 1H), 8.23-8.25 (d, 1H),8.27-8.30 (d, 1H). |
| 193 | 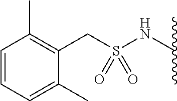 [SMILES: *NS(=O)(=O)Cc1c(C)cccc1C] | 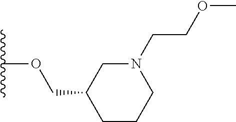 [SMILES: *OC[C@H]1CCCN(CCOC)C1] | GS-2 | LC-MS: 684.4 [M + H]⁺; ¹HNMR (CD₃OD, 400 MHz): δ 1.67-1.68 (m, 3H), 1.78-1.82 (d, 2H), 2.15 (s, 6H), 2.34-2.40 (m, 2H), 2.44-2.96 (m, 2H), 2.99-3.05 (m, 3H), 3.28-3.29 (d, 3H), 3.32-3.44 (m, 2H), 3.53-3.57 (m, 2H), 4.11-4.20 (m, 2H),4.33-4.37 (d, 4H), 6.36 (s, 1H), 6.49- 6.59 (m, 2H), 7.30-7.32 (d, 1H), 7.38 (s, |
| | | | | 1H), 7.55-7.62 (m, 3H), 7.78-7.80 (d, |
| | | | | 1H), 8.24-8.26 (d, 1H), 8.28-8.30 (d, |
| | | | | 1H). |
| 194 | 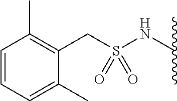 [SMILES: *NS(=O)(=O)Cc1c(C)cccc1C] | 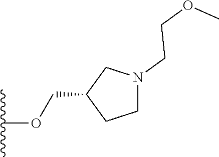 [SMILES: *OC[C@H]1CCN(CCOC)C1] | GS-2 | LC-MS: 670.5 [M + H]⁺; ¹HNMR (CD₃OD, 400 MHz): δ 2.16 (s, 6H), 2.64- 2.92 (m, 3H), 2.95-3.10 (m, 3H), 3.40 (s, 3H), 3.50-3.60 (m, 3H), 4.10-4.30 (m, 4H), 4.39 (s, 2H), 4.40-4.45 (m, 2H), 6.39 (s, 1H), 6.53 (s, 2H), 6.95-7.01 (m, 2H), 7.04-7.09 (m, 2H), 7.32-7.41 (m, 2H), 7.59-7.64(m, 2H), 7.79-7.83 (m, 1H), 8.27-8.32 (m, 2H). |
| 195 | 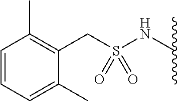 [SMILES: *NS(=O)(=O)Cc1c(C)cccc1C] | 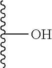 [SMILES: *O] | GS-2 | LC-MS: 529.1 [M + H]⁺; ¹HNMR (CD₃OD, 400 MHz): δ 2.22 (s, 6H), 4.32- 4.39 (m, 4H), 6.29 (s, 1H), 6.55 (s, 2H), 6.9-7.03 (m, 3H), 7.27-7.29 (m, 1H), 7.43 (s, 1H), 7.51-7.55 (m, 3H), 7.74-7.78 (m, 1H), 8.23-8.30(m, 2H). |
| 196 | 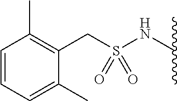 [SMILES: *NS(=O)(=O)Cc1c(C)cccc1C] | 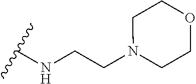 [SMILES: *NCCN1CCOCC1] | GS-2 | LC-MS: 641.0 [M + H]⁺; ¹HNMR (CD₃OD, 400 MHz): δ 2.19 (s, 6H), 2.95- 3.01 (m, 2H), 3.35-3.45 (m, 2H), 3.30- 3.34 (m, 2H), 3.45-3.55 (m, 2H), 3.63- 3.66 (m, 2H), 3.80-3.84 (m, 2H), 4.37 (s, 2H), 4.44 (s, 2H), 6.48 (s, 1H), 6.58 (s, 2H), 6.94-7.07 (m, 3H), 7.31-7.41 m, |
| | | | | 3H), 7.55-7.57 (m, 2H), 7.76-7.79 (m, |
| | | | | 1H), 8.22-8.28 (m, 2H). |
| 197 | 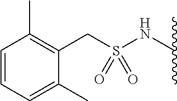 [SMILES: *NS(=O)(=O)Cc1c(C)cccc1C] | 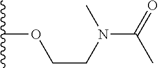 [SMILES: *OCCN(C)C(C)=O] | GS-2 | LC-MS: 628.5 [M + H]⁺; ¹HNMR (CD₃OD, 400 MHz): δ 1.90-2.04 (s, 3H), 2.19 (s, 6H), 2.95 (s, 3H), 3.56-3.62 (m, 2H), 4.29-4.38 (m, 6H), 6.29-6.31 (d, 1H), 6.52 (s, 2H), 6.92-6.94 (d, 2H), 7.00-7.04 (m, 1H), 7.27-7.31 (m, 1H), 7.41 (s, 1H), 7.50-7.58 (m, 3H), 7.75- |
| | | | | 7.77 (d, 1H), 8.22-8.30 (m, 2H). |
| 198 | 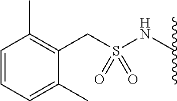 [SMILES: *NS(=O)(=O)Cc1c(C)cccc1C] | 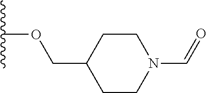 [SMILES: *OCC1CCN(C=O)CC1] | GS-2 | LC-MS: 654.5 [M + H]⁺; ¹HNMR (CD₃OD, 400 MHz): δ 0.80-1.00 (m, 2H), 1.40-1.55 (m, 2H), 1.60-1.65 (m, 1H),2.20 (s, 6H), 2.25-2.35 (m, 1H), 2.75-2.85 (m, 1H), 3.40-3.55 (m, 1H), 4.00 (s, 2H), 4.05-4.15 (m, 1H), 4.33 (s, 2H), 4.35 (s, 2H), 6.31 (s, 1H), 6.51 (s, |
| | | | | 2H), 6.93-6.95 (m, 2H), 7.01-7.05 (m, |
| | | | | 1H), 7.28-7.30 (d, 1H), 7.39 (s, 1H), |
| | | | | 7.53-7.59 (m, 3H), 7.76-7.79 (m, 1H), |
| | | | | 7.83 (s, 1H), 8.21-8.29 (m, 2H). |
| 199 | 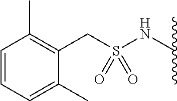 [SMILES: *NS(=O)(=O)Cc1c(C)cccc1C] | 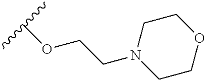 [SMILES: *OCCN1CCOCC1] | GS-2 | LC-MS: 642.0 [M + H]⁺; ¹HNMR (CD₃OD, 400 MHz): δ 2.14 (s, 6H), 2.95- 3.02 (t, 2H), 3.36-3.44 (m, 4H), 3.65- 3.87 (m, 4H), 4.35 (s, 2H), 4.41 (s, 2H), 4.54-4.55 (t, 2H), 6.40 (s, 1H), 6.58 (s, 2H), 6.96-6.98 (d, 2H), 7.04-7.07 (t, 1H), 7.32-7.34 (d, 1H), 7.46 (s, 1H), 7.59-7.63 |
| | | | | (b, 2H), 7.74 (s, 1H), 7.82 (d, 1H), 8.27- |
| | | | | 8.33 (t, 2H). |
| 200 | 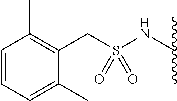 [SMILES: *NS(=O)(=O)Cc1c(C)cccc1C] | 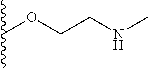 [SMILES: *OCCNC] | GS-2 | LC-MS: 586.4 [M + H]⁺; ¹HNMR (CD₃OD, 400 MHz): δ 2.16 (s, 6H), 2.64 (s, 3H), 3.26-3.28 (t, 2H), 4.36 (s, 2H), 4.45 (s, 2H), 4.46-4.47 (t, 2H), 6.40 (s, 1H), 6.57 (s, 2H), 6.95-6.96 (b, 2H), 7.05 (m, 1H), 7.33-7.35 (d, 1H), 7.42 (s, 1H), 7.57-7.61 (m, 2H), 7.73 (s, 1H), 7.80- |
| | | | | 7.82 (d, 1H), 8.26-8.33(m, 2H). |
| 201 | 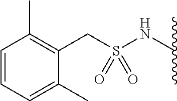 [SMILES: *NS(=O)(=O)Cc1c(C)cccc1C] | 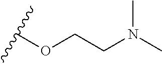 [SMILES: *OCCN(C)C] | GS-2 | LC-MS: 600.4 [M + H]⁺; ¹HNMR (CD₃OD, 400 MHz): δ 2.14 (s, 6H), 2.81 (s, 6H), 3.38-3.39 (b, 2H), 4.36 (s, 2H), 4.51 (s, 2H), 4.52-4.54 (b, 2H), 6.41 (s, 1H), 6.58 (s, 2H), 6.96-6.97 (b, 2H), 7.06-7.08 (m, 1H), 7.33-7.35 (d, 1H), 7.454 (s, 1H), 7.59-7.63 (m, 2H), 7.72 (s, |
| | | | | 1H), 7.83-7.84 (d, 1H), 8.27-8.33 (m, |
| | | | | 2H). |
| 202 | 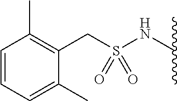 [SMILES: *NS(=O)(=O)Cc1c(C)cccc1C] | 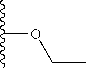 [SMILES: *OCC] | GS-1 | LC-MS: 557.3 [M + H]⁺; ¹HNMR(CD₃OD, 400 MHz): δ 1.11-1.14 (t, 3H), 2.20 (s, 6H), 4.17-4.19 (q, 2H), 4.34 (s, 2H), 4.38 (s, 2H), 6.29 (s, 1H), 6.53 (s, 2H), 6.93-6.95 (d, 2H), 7.01-7.05 (m, 1H), 7.28-7.30 (d, 1H), 7.42 (s, 1H), 7.53-7.56 (m, 3H), 7.79-7.80 (m, 1H), |
| | | | | 8.25-8.29 (m, 2H). |
| 203 | 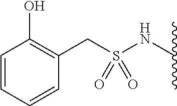 [SMILES: *NS(=O)(=O)Cc1ccccc1O] | 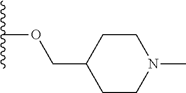 [SMILES: *OCC1CCN(C)CC1] | GS-2 | LC-MS: 626.1 [M + H]⁺; ¹HNMR (CD₃OD, 400 MHz): δ 1.38-1.40 (m, 2H), 1.69-1.72 (d, 3H), 2.63-2.65 (t, 3H), 2.79(s, 3H), 3.30-3.36(d, 2H), 4.08-4.09 (d, 2H), 4.27(s, 2H),4.36 (s, 2H), 6.30 (s, 1H), 6.54 (s, 2H), 6.69-6.76 (m, 2H), 7.00-7.02 (d, 1H), 7.09-7.13 (t, 1H), |
| | | | | 7.30-7.32 (d, 1H),7.41(s, 1H), 7.55-7.59 |
| | | | | (m, 3H), 7.78-7.81 (t, 1H), 8.21-8.24 (d, |
| | | | | 1H), 8.29-8.30 (d, 1H). |
| 204 | 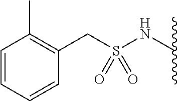 [SMILES: *NS(=O)(=O)Cc1ccccc1C] | 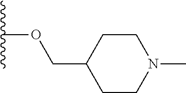 [SMILES: *OCC1CCN(C)CC1] | GS-2 | LC-MS: 626.4 [M + H]⁺; ¹HNMR (CD₃OD, 400 MHz): δ 1.34-1.43 (q, 2H), 1.7-1.78 (d, 2H), 2.19 (s, 3H), 2.65-2.72 (t, 2H), 2.75 (s, 3H), 3.00 (bs, 1H) 3.33- 3.36 (d, 2H), 4.07-4.08 (d, 2H), 4.24 (s, 2H), 4.34 (s, 2H), 6.29 (s, 1H), 6.52 (s, 2H), 6.92-6.94 (d, 1H), 6.98-7.00 (t, 1H), |
| | | | | 7.09-7.16 (m, 2H), 7.29-7.31 (d, 1H), |
| | | | | 7.37 (s, 1H), 7.53-7.58 (m, 3H), 7.77- |
| | | | | 7.79 (m, 1H), 8.21-8.24 (m, 1H), 8.24- |
| | | | | 8.29 (d, 1H). |
| 205 | 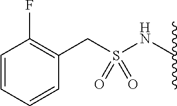 [SMILES: *NS(=O)(=O)Cc1ccccc1F] | 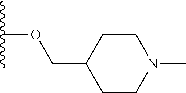 [SMILES: *OCC1CCN(C)CC1] | GS-2 | LC-MS: 630.4 [M + H]⁺; ¹HNMR (CD₃OD, 300 MHz): δ 1.29-1.43 (m, 2H), 1.61-1.75 (m, 3H), 2.65-2.77 (m, 3H), 3.29-3.38 (m, 3H), 4.10-4.12 (d, 2H), 4.29 (s, 2H), 4.36 (s, 2H), 6.22 (s, 1H), 6.54 (s, 2H), 6.98-7.00 (d, 1H), 7.04-7.06 (d, 1H), 7.08-7.10 (d, 1H), 7.20-7.26 (m, |
| | | | | 1H), 7.32-7.34 (d, 2H), 7.43 (s, 1H), |
| | | | | 7.58-7.61 (t, 3H), 7.80-7.83 (t, 1H), 8.23- |
| | | | | 8.26 (t, 1H), 8.29-8.31 (d, 1H). |
| 206 | 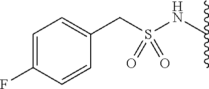 [SMILES: *NS(=O)(=O)Cc1ccc(F)cc1] | 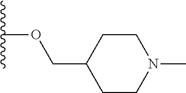 [SMILES: *OCC1CCN(C)CC1] | GS-2 | LC-MS: 630.4 [M + H]⁺; ¹HNMR (CD₃OD, 400 MHz): δ 1.39-1.42 (m, 2H), 1.72-7.76 (d, 3H), 2.62-2.76 (m, 5H), 3.32-3.38 (m, 2H), 4.08 (m, 2H), 4.18 (s, 2H), 4.29-4.38 (t, 2H), 6.23 (s, 1H), 6.52 (s, 2H), 6.90-6.95 (t, 2H), 7.04-7.09 (m, |
| | | | | 2H), 7.30-7.32 (d, 2H), 7.41 (s, 1H), |
| | | | | 7.54-7.59 (m, 2H), 7.79-7.82 (m, 1H), |
| | | | | 8.21-8.24 (m, 1H), 8.27-8.29 (d, 1H). |
| 207 | 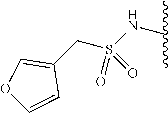 [SMILES: *NS(=O)(=O)Cc1ccoc1] | 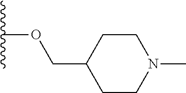 [SMILES: *OCC1CCN(C)CC1] | GS-2 | LC-MS: 602.0 [M + H]⁺; ¹HNMR (CD₃OD, 400 MHz): δ 1.27-1.45 (m, 2H), 1.76-1.78 (t, 3H), 2.70-2.85 (m, 4H), 3.08-3.10 (d, 2H), 3.34-3.38 (d, 2H), 4.06-3.08 (t, 2H), 4.16 (s, 1H), 4.34 (s, 2H), 6.18-6.22 (d, 2H), 6.51 (s, 2H), 7.19 (s, 1H), 7.28-7.32 (d, 1H), 7.32-7.34 (d, 1H), 7.50-7.52 (d, 1H), 7.56-7.59 (m, |
| | | | | 3H), 7.80-7.82 (t, 1H), 8.20-8.24 (t, 1H), |
| | | | | 8.25-8.29 (d, 1H). |
| 208 | 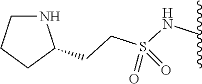 [SMILES: *NS(=O)(=O)CC[C@@H]1CCCN1] | 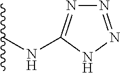 [SMILES: *Nc1nnn[nH]1] | GS-2 | LC-MS: 575.3 [M + H]⁺;¹HNMR (CD₃OD, 300 MHz): δ 1.40-1.42 (t, 1H), 1.86-2.14 (m, 4H), 3.01-3.11 (m, 3H), 3.22-3.24 (t, 2H), 4.38 (s, 2H), 6.51 (s, 1H), 6.59 (s, 2H), 7.32-7.34 (d, 1H), 7.47-7.57 (m, 3H), 7.70 (s, 1H), 7.80- |
| | | | | 7.82 (t, 2H), 8.20-8.27 (m, 1H), 8.30- |
| | | | | 8.36 (d, 1H). |
| 209 | 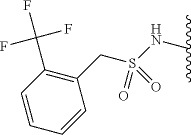 [SMILES: *NS(=O)(=O)Cc1ccccc1C(F)(F)F] | 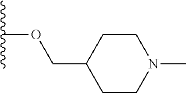 [SMILES: *OCC1CCN(C)CC1] | GS-2 | LC-MS: 680.2 [M + H]⁺; ¹HNMR (CD₃OD, 400 MHz): δ 1.02-1.05 (t, 2H), 1.41-1.43 (d, 2H), 1.73-1.75 (d, 3H), 1.99 (s, 1H), 2.70-2.91 (m, 4H), 4.06-4.08 (d, 2H), 4.33 (s, 2H), 4.42 (s, 2H), 6.30 (s, 1H), 6.51 (s, 2H), 6.82-6.84 (d, 1H), 7.29-7.31 (t, 2H), 7.41-7.49 (m, 6H), 7.58-7.66 (m, 2H), 7.75-7.79 (t, 1H). |
| 210 | 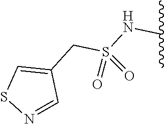 [SMILES: *NS(=O)(=O)Cc1cnsc1] | 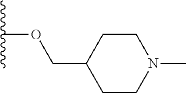 [SMILES: *OCC1CCN(C)CC1] | GS-2 | LC-MS: 619.3 [M + H]⁺; ¹HNMR (CD₃OD, 400 MHz): δ 1.40-1.44 (m, 2H), 1.77-1.82 (m, 2H), 2.7-2.80 (m, 6H), 3.38-3.43 (m, 2H), 4.10-4.12 (d, 2H), 4.39 (s, 2H), 4.44 (s, 2H), 6.11 (s, 1H), 6.52 (s, 2H), 7.31-7.34 (d, 1H), 7.50 (s, 1H), 7.58-7.61 (m, 3H), 7.83-7.86 (m, 1H), 8.08 (s, 1H), 8.22-8.25 (m, 1H), |
| | | | | 8.27-8.30 (d, 1H), 8.62 (s, 1H). |
| 211 | 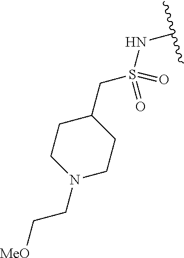 [SMILES: *NS(=O)(=O)CC1CCN(CCOC)CC1] | 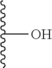 [SMILES: *O] | GS-1 | LC-MS: 566.1 [M + H]⁺; ¹HNMR (CD₃OD, 300 MHz): δ 1.58-1.60 (m, 1H), 2.03-2.04 (m, 5H), 2.94-2.96 (m, 6H), 3.39 (s, 3H), 3.52-3.54 (t, 2H), 3.67-3.69 (t, 2H), 4.34 (s, 2H), 6.23 (s, 1H), 6.54 (s, 1H) 7.28-7.31 (d, 1H), 7.48-7.58 (m, 4H), 7.80-8.82 (t, 1H), 8.26-8.29 (m, 2H). |
| 212 | 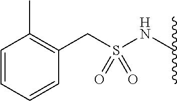 [SMILES: *NS(=O)(=O)Cc1ccccc1C] | 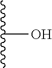 [SMILES: *O] | GS-1 | LC-MS: 513.1 [M − H]⁻; ¹HNMR(CD₃OD, 300 MHz): 2.21 (s, 3H), 4.28-4.33 (d, 4H), 6.40 (s, 1H), 6.56 (s, 2H), 6.93-6.95 (d, 1H), 7.01-7.09 (m, 1H), 7.11-7.15 (m, 2H), 7.27-7.29 (d, 1H), 7.45 (s, 1H), 7.54-7.56 (m, 3H), 7.76-7.79 (m, 1H), 8.22-8.29 (m, 2H). |
| 213 | 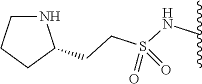 [SMILES: *NS(=O)(=O)CC[C@@H]1CCCN1] | 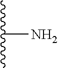 [SMILES: *N] | GS-1 | LC-MS: 507.3 [M + H]⁺; ¹HNMR (CD₃OD, 400 MHz): δ 1.49-1.55 (m, 1H), 1.92-2.02 (m, 3H); 2.08-2.14 (m, 2H), 3.11-3.15 (t, 2H), 3.22-3.27 (m, 2H), 3.48-3.52 (m, 1H), 4.23 (s, 2H), 6.43- 6.43 (bs, 1H), 6.50 (s, 2H), 7.26-7.28 (d, |
| | | | | 1H), 7.35 (s, 1H), 7.52-7.54 (m, 2H), |
| | | | | 7.55-7.56 (d, 1H), 7.81-7.83 (m, 1H), |
| | | | | 8.19-8.23 (m, 2H). |
| 214 | 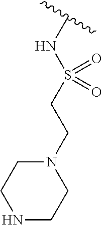 [SMILES: *NS(=O)(=O)CCN1CCNCC1] | 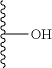 [SMILES: *O] | GS-1 | LC-MS: 523.3 [M + H]⁺; ¹HNMR (CD₃OD, 300 MHz): δ 2.56-2.58 (d, 4H), 2.72-2.74 (t, 2H), 3.00-3.02 (t, 4H), 3.16- 3.18 (t, 2H), 4.34 (s, 2H), 6.29 (s, 1H), 6.54 (s, 2H), 7.27-7.29 (d, 1H), 7.45 (s, 1H), 7.52-7.58 (m, 3H), 7.80-7.82 (d, 1H), 8.25-8.27 (d, 2H). |
| i215 | 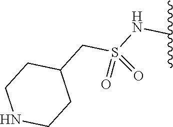 [SMILES: *NS(=O)(=O)CC1CCNCC1] | 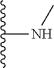 [SMILES: *NC] | GS-1 | LC-MS: 521.3 [M + H]⁺; ¹HNMR (CD₃OD, 400 MHz): δ 1.30-1.50 (m, 4H), 2.00-2.20 (m, 3H), 2.78 (s, 3H), 2.90- 3.00 (m, 5H), 4.32 (s, 2H), 6.43 (s, 1H), 6.48 (s, 2H), 7.25-7.27 (d, 1H), 7.48 (s, 1H), 7.52-7.57 (m, 2H), 7.75-7.95 (m, 1H), 8.19-8.21 (m, 2H). |
| 216 | 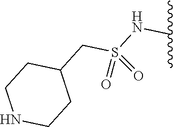 [SMILES: *NS(=O)(=O)CC1CCNCC1] | 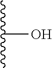 [SMILES: *O] | GS-1 | LC-MS: 508.4 [M + H]⁺;¹HNMR (CD₃OD, 400 MHz): δ 1.44-1.47 (m, 2H), 1.99-2.2 (m, 3H), 2.86-2.99 (m, 5H), 3.35 (bs, 1H), 4.35 (s, 2H), 6.24 (s, 1H), 6.55 (s, 2H), 7.29-7.31 (d, 1H), 7.50-7.58 (m, 4H), 7.82-7.84 (m, 1H), 8.23-8.29 (m, 2H). |
| 217 | 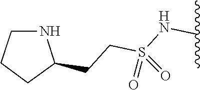 [SMILES: *NS(=O)(=O)CC[C@H]1CCCN1] | 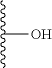 [SMILES: *O] | GS-1 | LC-MS: 508.1 [M + H]⁺;¹HNMR (CD₃OD, 400 MHz): δ 1.50-1.60 (m, 1H), 1.93-2.05 (m, 3H), 2.06-2.20 (m, 2H), 3.10-3.14 (m, 2H), 3.14-3.31 (m, 2H), 3.32-3.60 (m, 1H), 4.36 (s, 2H), 6.27 (s, 1H), 6.55 (s, 2H), 7.29-7.31 (d, 1H), 7.53- |
| | | | | 7.61 (m, 4H), 7.82-7.85 (t, 1H), 8.24- |
| | | | | 8.29 (m, 2H). |
| 218 | 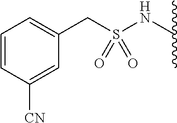 [SMILES: *NS(=O)(=O)Cc1cccc(C#N)c1] | 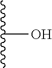 [SMILES: *O] | GS-2 | LC-MS: 526.3 [M + H]⁺; ¹HNMR (CD₃OD, 400 MHz): 4.31 (s, 2H), 4.33 (s, 2H), 6.25 (s, 1H), 6.56 (s, 2H), 7.27-7.44 (m, 4H), 7.52-7.62 (m, 5H), 7.78-7.81 (m, 1H), 8.22-8.28 (m, 2H). |
| 219 | 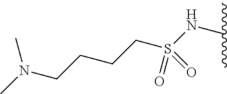 [SMILES: *NS(=O)(=O)CCCCN(C)C] | 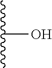 [SMILES: *O] | GS-3 | LC-MS: 510 [M + H]⁺. |
| 220 | 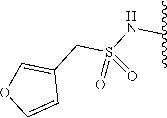 [SMILES: *NS(=O)(=O)Cc1ccoc1] | 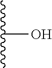 [SMILES: *O] | GS-2 | LC-MS: 491.3 [M + H]⁺; ¹HNMR (CD₃OD, 400 MHz): δ 4.08 (s, 2H), 4.33 (s, 2H), 6.18-6.20 (d, 2H), 6.55 (s, 2H), 7.23 (s, 1H), 7.27-7.29 (d, 1H), 7.34-7.38 (d, 1H), 7.53-7.57 (m, 4H), 7.80-7.82 (t, 1H), 8.20-8.24 (t, 1H), 8.26-8.28 (d, 1H). |
| 221 | 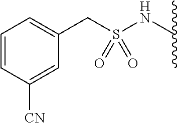 [SMILES: *NS(=O)(=O)Cc1cccc(C#N)c1] | 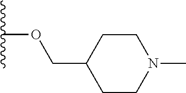 [SMILES: *OCC1CCN(C)CC1] | GS-2 | LC-MS: 637.4 [M + H]⁺; ¹HNMR (CD₃OD, 400 MHz): δ 1.38-1.41 (d, 3H), 1.73-1.75 (d, 2H), 2.71-2.77 (m, 5H), 3.37-3.40 (d, 2H), 4.08-4.10 (d, 2H), 4.30(s, 2H), 4.35-4.37 (d, 2H), 6.24 (s, 1H), 6.53 (s, 2H), 7.30-7.32 (d, 1H), 7.38-7.42 (m, 4H), 7.56-7.60 (m, 4H), 7.79-7.82 (t, 1H), 8.22-8.24 (t, 1H), |
| | | | | 8.27-8.29 (d, 1H). |
| 222 | 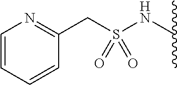 [SMILES: *NS(=O)(=O)Cc1ccccn1] | 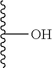 [SMILES: *O] | GS-2 | LC-MS: 502.0 [M + H]⁺; ¹HNMR (CD₃OD, 400 MHz): δ 4.20 (s, 2H), 4.37 (s, 2H), 6.25 (s, 1H), 6.59 (s, 2H), 7.14- 7.16 (d, 2H), 7.23-7.25 (m, 2H), 7.50- 7.51 (m, 2H)), 7.53 (s, 1H), 7.69 (m, 1H), 7.70 (m, 1H), 8.09-8.11 (d, 1H), 8.26- |
| | | | | 8.27 (m, 2H). |
| 223 | 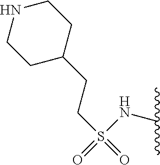 [SMILES: *NS(=O)(=O)CCC1CCNCC1] | 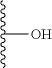 [SMILES: *O] | GS-1 | LC-MS: 522.3 [M + H]⁺; ¹HNMR (CD₃OD, 400 MHz): δ 1.21-1.26 (m, 3H), 1.55-1.58 (t, 3H), 1.72-1.76 (d, 2H), 2.80-2.86 (m, 3H), 2.94-2.98 (t, 2H), 4.32 (s, 2H), 6.21 (s, 1H),6.54 (s, 2H), 7.26-7.28 (d, 1H), 7.47 (s, 2H), 7.51-7.56 (m, 2H), 7.78-7.80 (d, 1H), 8.20-8.25 (m, 2H). |
| 224 | 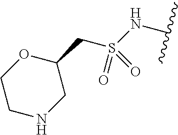 [SMILES: *NS(=O)(=O)C[C@H]1CNCCO1] | 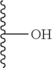 [SMILES: *O] | GS-2 | LC-MS: 509 [M + H]⁺; ¹HNMR (CD₃OD, 300 MHz): δ 2.85 (t, 1H), 3.03 (m, 1H), 3.15-3.17 (m, 3H), 3.23-3.31 (m, 1H), 3.57 (t, 1H), 3.77 (dd, 1H), 4.09-4.13 (m, 3H), 6.19 (s, 1H), 6.38 (d, 1H), 6.45 (d, 1H), 7.24 (d, 1H), 7.44 (s, 1H), 7.25 (s, 1H), 7.57-7.59 (m, 2H), 7.60-7.62 (m, 1H), 7.85 (d, 2H), 12.33-12.34 (d, 1H). |
| 225 | 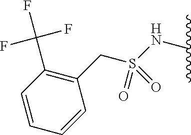 [SMILES: *NS(=O)(=O)Cc1ccccc1C(F)(F)F] | 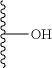 [SMILES: *O] | GS-2 | LC-MS: 569.0 [M + H]⁺; ¹HNMR (CD₃OD, 400 MHz): δ 4.31 (s, 2H), 4.43 (s, 2H), 6.23 (s, 1H), 6.55 (s, 2H), 7.27- 7.28 (d, 1H), 7.38-7.39 (d, 1H), 7.47-7.48 (t, 1H), 7.52 (s, 2H), 7.53-7.56 (m, 3H), 7.65-7.66 (d, 1H), 7.78-7.79 (t, 1H), 8.21-8.24 (m, 1H), 8.27-8.29 (d, 1H). |
| 226 | 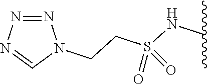 [SMILES: *NS(=O)(=O)CCn1cnnn1] | 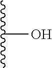 [SMILES: *O] | GS-3 | LC-MS: 507 [M + H]⁺. |
| 227 | 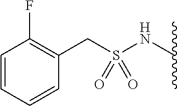 [SMILES: *NS(=O)(=O)Cc1ccccc1F] | 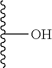 [SMILES: *O] | GS-1 | LC-MS: 519.0 [M + H]⁺; ¹HNMR (CD₃OD, 400 MHz): δ 4.30 (s, 2H), 4.32 (s. 2H), 6.19 (s, 1H), 6.55 (s, 2H), 7.00- 7.14 (m, 3H), 7.26-7.28 (d, 2H), 7.48- 7.55 (m, 4H), 7.60-7.76 (m, 1H), 8.25- 8.27 (m, 2H) |
| 228 | 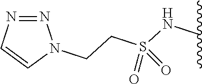 [SMILES: *NS(=O)(=O)CCn1ccnn1] | 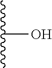 [SMILES: *O] | GS-3 | LC-MS: 506 [M + H]⁺. |
| 229 | 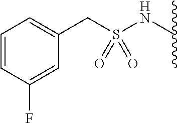 [SMILES: *NS(=O)(=O)Cc1cccc(F)c1] | 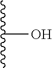 [SMILES: *O] | GS-2 | LC-MS: 519.0 [M + H]⁺; ¹HNMR (CD₃OD, 400 MHz): δ 4.23 (s, 2H),4.32 (s, 2H),6.25 (s, 1H), 6.56 (s, 2H), 6.83 (d, 1H), 6.93-6.98 (m, 1H), 7.00-7.03 (m, 1H), 7.21-7.26 (m, 1H), 7.27 (d, 1H), 7.44 (s, 1H), 7.50 (s, 1H), 7.54-7.56 (m, 2H), 7.78-7.81 (m, 1H), 8.22-8.27 (m, 2H). |
| 230 | 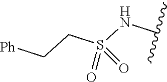 [SMILES: *NS(=O)(=O)CCc1ccccc1] | 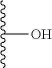 [SMILES: *O] | GS-2 | LC-MS: 515.2 [M + H]⁺; ¹HNMR (CD₃OD, 400 MHz): δ 2.83-2.87 (m, 2H), 3.10-3.14 (m, 2H), 4.28 (s, 2H), 6.272- 6.276 (s, 1H), 6.54 (s, 2H), 7.00-7.02 (d, 2H), 7.15-7.24 (m, 4H), 7.43 (s, 1H), 7.47 (s, 1H), 7.53-7.56 (m, 2H), 7.79- |
| | | | | 7.81 (m, 1H), 8.21-8.24 (m, 2H), |
| 231 | 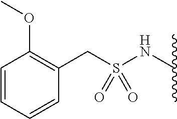 [SMILES: *NS(=O)(=O)Cc1ccccc1OC] | 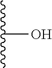 [SMILES: *O] | GS-2 | LC-MS: 531.1 [M + H]⁺; ¹HNMR (CD₃OD, 400 MHz): δ 3.28-3.30 (m, 2H), 4.32-4.33 (d, 3H), 4.89 (s, 2H), 6.19 (s, 1H), 6.57 (s, 2H), 6.76-6.77 (d, 1H), 6.82-6.86 (t, 1H), 7.13-7.15 (dd, 1H), 7.20-7.22 (m, 1H), 7.26-7.28 (d, 1H), 7.32 (s, 1H), 7.51-7.53 (t, 3H), 7.72-7.74 (m, 1H), 8.20-8.22 (m, 1H), 8.25-8.27 (d, |
| | | | | 1H). |
| 232 | 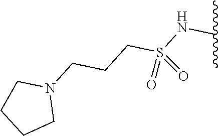 [SMILES: *NS(=O)(=O)CCCN1CCCC1] | 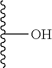 [SMILES: *O] | GS-3 | LC-MS: 522 [M + H]⁺. |
| 233 | 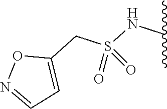 [SMILES: *NS(=O)(=O)Cc1ccno1] | 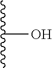 [SMILES: *O] | GS-3 | LC-MS: 492 [M + H]⁺. |
| 234 | 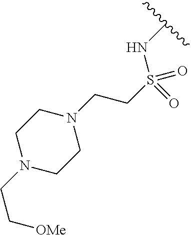 [SMILES: *NS(=O)(=O)CCN1CCN(CCOC)CC1] | 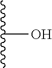 [SMILES: *O] | GS1 | LC-MS: 581.1 [M + H]⁺; ¹HNMR (CD₃OD, 400 MHz): δ 2.72-2.75 (m, 4H), 2.80-3.05 (m, 4H), 3.09-3.11 (m, 3H), 3.16-3.19 (m, 3H), 3.35(m, 3H), 3.59- 3.61 (m, 2H), 4.34(s, 2H), 6.31 (s, 1H), 6.54 (s, 2H), 7.28-7.30 (d, 1H), 7.43 (m, 1H), 7.53 (s, 1H), 7.55-7.58 (m, 2H), 7.82-7.84 (m, 1H), 8.23-8.28 (m, 2H). |
| 235 | 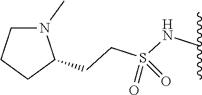 [SMILES: *NS(=O)(=O)CC[C@@H]1CCCN1C] | 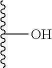 [SMILES: *O] | GS-1 | LC-MS: 522.3 [M + H]⁺; ¹HNMR (CD₃OD, 400 MHz): δ 1.53-1.58 (m, 2H), 1.81-1.87 (m, 2H), 1.96-2.07 (m, 2H), 2.17-2.28 (m, 2H), 2.79 (s, 3H), 3.11- 3.15 (t, 3H), 3.62-3.63 (m, 1H), 4.34 (s, 2H), 6.32 (s, 1H), 6.55 (s, 2H), 7.27-7.29 |
| | | | | (d, 1H), 7.46 (s, 1H), 7.54 (s, 1H), 7.56- |
| | | | | 7.59 (m, 1H), 7.83-7.85 (m, 1H), 8.23- |
| | | | | 8.25 (d, 1H), 8.27 (s, 1H). |
| 236 | 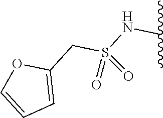 [SMILES: *NS(=O)(=O)Cc1ccco1] | 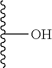 [SMILES: *O] | GS-2 | LC-MS: 491.3 [M + H]⁺; ¹HNMR (CD₃OD, 400 MHz): δ 4.29 (s, 2H), 4.33 (s, 2H), 6.15-6.17 (d, 1H), 6.21 (s, 1H), 6.28-6.34 (m, 1H), 6.56 (s, 2H), 7.28- 7.29 (d, 1H), 7.33-7.35 (d, 1H), 7.53-7.59 (m, 4H), 7.81-7.84 (m, 1H), 8.23-8.26 (m, 1H), 8.28-8.30 (d, 1H). |
| 237 | 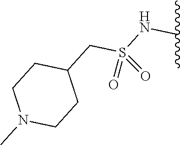 [SMILES: *NS(=O)(=O)CC1CCN(C)CC1] | 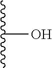 [SMILES: *O] | GS-1 | LC-MS: 522.1 [M + H]⁺; ¹HNMR (CD₃OD, 400 MHz): δ 1.48-1.52 (m, 2H), 2.03-2.06 (d, 3H), 2.82 (s, 3H), 2.91-2.95 (m, 3H), 3.44-3.47 (d, 2H), 4.33 (s, 3H), 6.21 (s, 1H), 6.53 (s, 2H), 7.27-7.29 (d, 1H), 7.47 (s, 1H), 7.51 (s, 1H), 7.54-7.56 (m, 2H), 7.79 (s, 1H), 8.20-8.27 (m, 2H). |
| 238 | 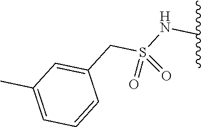 [SMILES: *NS(=O)(=O)Cc1cccc(C)c1] | 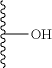 [SMILES: *O] | GS-2 | LC-MS: 515.1 [M + H]⁺; ¹HNMR (CD₃OD, 400 MHz): δ 2.20 (s, 3H), 4.15 (s, 2H), 4.31 (s, 2H), 6.28 (s, 1H), 6.55 (s, 2H), 6.80-6.82 (d, 1H),6.90 (s, 1H), 7.05-7.11 (m, 2H), 7.25-7.27 (d, 1H), 7.36 (s, 1H), 7.51-7.55 (m, 3H), 7.75- 7.77 (d, 1H), 8.21-8.25 (m, 2H). |
| 239 | 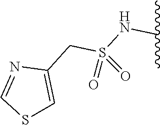 [SMILES: *NS(=O)(=O)Cc1cscn1] | 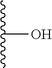 [SMILES: *O] | GS-3 | LC-MS: 508 [M + H]⁺. |
| 240 | 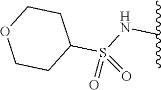 [SMILES: *NS(=O)(=O)C1CCOCC1] | 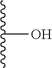 [SMILES: *O] | GS-3 | LC-MS: 495 [M + H]⁺. |
| 241 | 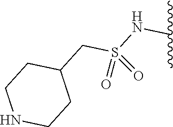 [SMILES: *NS(=O)(=O)CC1CCNCC1] | 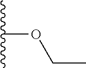 [SMILES: *OCC] | GS-2 | LC-MS: 536.4 [M + H]⁺; ¹HNMR (CD₃OD, 400 MHz): δ 1.10-1.13 (t, 3H), 1.43-1.46 (m, 3H), 2.01-2.04 (d, 3H), 2.14-2.15 (m, 2H), 2.91-2.95 (t, 3H), 4.15-4.21 (q, 2H), 4.34 (s, 2H), 6.23 (s, 1H), 6.52 (s, 2H), 7.29-7.31 (d, 1H), 7.48 (bs, 1H), 7.53 (s, 1H), 7.56-7.58 (m, 2H), 7.82-7.84 (m, 1H), 8.23-8.25 (d, 1H), |
| | | | | 8.27-8.29 (d, 1H). |
| 242 | 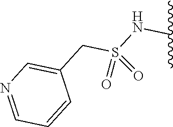 [SMILES: *NS(=O)(=O)Cc1cccnc1] | 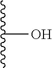 [SMILES: *O] | GS-2 | LC-MS: 502.0 [M + H]⁺; ¹HNMR (CD₃OD, 400 MHz): δ 4.36-4.37 (m, 4H), 6.16 (s, 1H), 6.54 (s, 2H), 7.27-7.8 (m, 8H), 8.192-8.4 (m, 4H). |
| 243 | 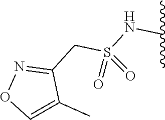 [SMILES: *NS(=O)(=O)Cc1nocc1C] | 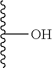 [SMILES: *O] | GS-3 | LC-MS: 506 [M + H]⁺. |
| 244 | 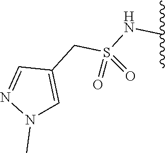 [SMILES: *NS(=O)(=O)Cc1cnn(C)c1] | 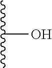 [SMILES: *O] | GS-2 | LC-MS: 503.3 [M + H]⁺; ¹HNMR (DMSO-d₆, 400 MHz): δ 3.72 (s, 3H), 4.18 (s, 2H), 4.28 (s, 2H), 6.17 (s, 1H), 6.45 (s, 2H), 7.12 (s, 1H), 7.33-7.35 (d, 1H), 7.41 (s, 1H), 7.50 (s, 2H), 7.57-7.59 (d, 2H), 7.87-7.90 (m, 1H), 8.20-8.25 (m, 3H), 8.32-8.34 (d, 1H), 9.76 (s, 1H), 13.17 (s, 1H). |
| 245 | 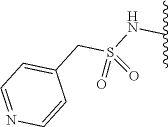 [SMILES: *NS(=O)(=O)Cc1ccncc1] | 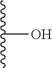 [SMILES: *O] | GS-2 | LC-MS: 502.3 [M + H]⁺; ¹HNMR (CD₃OD, 400 MHz): δ 4.33 (s, 2H), 4.47 (s, 1H), 4.87 (s, 1H), 6.18 (s, 1H), 6.54 (s, 2H), 7.28-7.30 (d, 1H), 7.47-7.51 (m, 3H), 7.55-7.58 (m, 3H), 7.79-7.81 (d, 1H), 8.22-8.28 (m, 2H), 8.50-8.52 (d, 2H) |
| 246 | 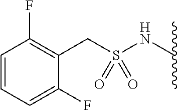 [SMILES: *NS(=O)(=O)Cc1c(F)cccc1F] | 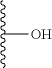 [SMILES: *O] | GS-2 | LC-MS: 537.2 [M + H]⁺; ¹HNMR (CD₃OD, 400 MHz): δ 4.32 (s, 2H), 4.36 (s, 2H), 6.14 (s, 1H), 6.56 (s, 2H), 6.88- 6.89 (t, 2H), 7.27-7.29 (d, 2H), 7.53-7.49 (m, 4H), 7.73 (s, 1H), 8.19 (s, 1H), 8.25 (s, 1H). |
| 247 | 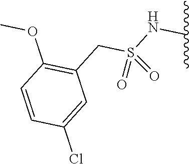 [SMILES: *NS(=O)(=O)Cc1cc(Cl)ccc1OC] | 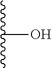 [SMILES: *O] | GS-2 | LC-MS: 566.3 [M + H]⁺; ¹HNMR (CD₃OD, 400 MHz): δ 3.20 (s, 3H), 4.32 (s, 4H), 6.19 (s, 1H), 6.57 (s, 2H), 6.72- 6.74 (d, 1H), 7.19-7.25 (m, 2H), 7.26 (s,1H), 7.28 (bs, 1H), 7.51-7.54 (m, 3H), 7.73-7.75 (m, 1H), 8.20-8.27 (m, 2H). |
| 248 | 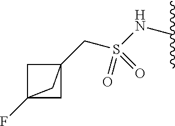 [SMILES: *NS(=O)(=O)CC12CC(F)(C1)C2] | 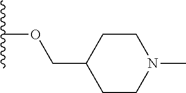 [SMILES: *OCC1CCN(C)CC1] | GS-2 | LC-MS: 620.5 [M + H]⁺; ¹HNMR (DMSO-d₆, 400 MHz): δ 1.34-1.38 (m, 4H), 1.72-1.75 (m, 4H), 1.91-1.92 (d, 4H), 2.72 (s, 4H), 3.35-3.39 (m, 2H), 3.43 (s, 2H), 3.99-4.01 (m, 2H), 4.28 (s, 2H), 6.06 (s, 1H), 6.43 (s, 2H), 7.37- 7.392 (d, 1H), 7.52 (s, 1H), 7.58-7.60 (m, 1H), 7.62 (s, 1H), 7.89-7.91 (m, 1H), |
| | | | | 8.21-8.22 (m, 3H), 8.35-8.37 (d, 1H), |
| | | | | 9.25 (bs, 1H), 9.80 (bs, 1H). |
| 249 | 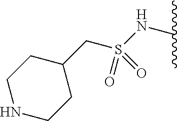 [SMILES: *NS(=O)(=O)CC1CCNCC1] | 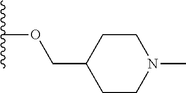 [SMILES: *OCC1CCN(C)CC1] | GS-2 | LC-MS: 619.0 [M + H]⁺; ¹HNMR (CD₃OD, 400 MHz): δ 1.40-1.49 (m, 4H), 1.63-1.79 (m, 4H), 1.99-2.01 (d, 2H), 2.13 (bs, 2H), 2.70-2.83 (m, 4H), 2.89-2.99 (m, 4H), 3.28-3.33 (m, 2H), 3.38-3.45 (m, 2H), 4.09-4.11 (d, 2H), 4.34 (s, 2H), 6.22 (s, 1H), 6.51 (s, 2H), 7.30-7.32 (d, 1H), 7.49 (s, 1H), 7.57-7.60 |
| | | | | (t, 2H), 7.84-7.88 (t, 1H), 8.16-8.23 (m, |
| | | | | 1H), 8.27-8.28 (d, 1H). |
| 250 | 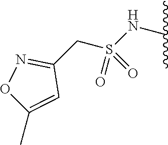 [SMILES: *NS(=O)(=O)Cc1cc(C)on1] | 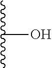 [SMILES: *O] | GS-2 | LC-MS: 506.3 [M + H]⁺; ¹HNMR (CD₃OD, 400 MHz): δ 2.24 (s, 3H), 4.25 (s, 2H), 4.34 (s, 2H), 6.03 (s, 1H), 6.19 (s, 1H), 6.54 (s, 2H), 7.26-7.28 (d, 1H), 7.48-7.59 (m, 4H), 7.80-7.83 (d, 1H), 8.21-8.25 (m, 2H). |
| 251 | 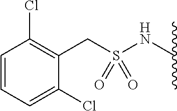 [SMILES: *NS(=O)(=O)Cc1c(Cl)cccc1Cl] | 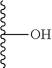 [SMILES: *O] | GS-2 | LC-MS: 569.2 [M + H]⁺; ¹HNMR (CD₃OD, 400 MHz): δ 4.33 (s, 2H), 4.71 (s, 2H), 6.15 (s, 1H), 6.54 (s, 2H), 7.16- 7.14 (m, 1H), 7.27-7.29 (d, 3H), 7.48- 7.52 (m, 4H), 7.72-7.74 (m, 1H), 8.18- 8.20 (m, 1H), 8.26-8.28 (d, 1H). |
| 252 | 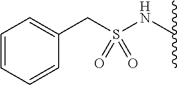 [SMILES: *NS(=O)(=O)Cc1ccccc1] | 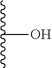 [SMILES: *O] | GS-2 | LC-MS: 500.3 [M + H]⁺; ¹HNMR (CD₃OD, 400 MHz): δ 4.18 (s, 2H), 4.25 (s, 2H), 4.28 (s, 2H), 6.34 (s, 2H), 6.56 (s, 2H), 7.23-7.34 (m, 4H), 7.50 (s, 2H), 7.60-7.70 (m, 2H), 7.76 (s, 1H), 7.92- 7.94 (d, 1H), 8.24-8.30 (m,2H). |
| 253 | 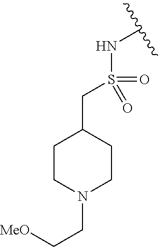 [SMILES: *NS(=O)(=O)CC1CCN(CCOC)CC1] | 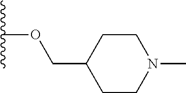 [SMILES: *OCC1CCN(C)CC1] | GS-2 | LC-MS: 677.5 [M + H]⁺; ¹HNMR (CD₃OD, 400 MHz): δ 1.39-1.43 (m, 3H), 1.52-1.55 (m, 2H), 1.71-1.78 (m, 3H), 2.03-2.06 (d, 2H), 2.70-2.78 (m, 5H), 2.91-2.97 (m, 4H), 3.25-3.29 (m, 2H), 3.37 (m, 5H), 3.54-3.57 (d, 2H), 3.66- 3.68 (t, 2H), 4.08-4.09 (d, 2H), 4.34 (s, 2H), 6.22 (s, 1H), 6.51 (s, 2H), 7.29-7.31 (d, 1H), 7.49 (s, 1H), 7.56-7.58 (m, 3H), 7.83-7.85 (t, 1H), 8.21-8.23 (t, 1H), 8.26- 8.28 (d, 1H). |
| 254 | 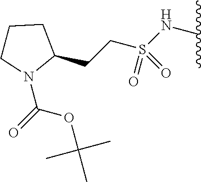 [SMILES: *NS(=O)(=O)CC[C@@H]1CCCN1C(=O)OC(C)(C)C] | 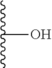 [SMILES: *O] | GS-1 | LC-MS: 608.3 [M + H]⁺; ¹HNMR (DMSO-d₆, 300 MHz): δ 1.18-1.30 (m, 12H), 1.63 (m, 3H), 2.91-2.96 (m, 2H), 3.15 (bs, 1H), 4.22-4.24 (d, 3H), 5.05 (bs, 2H), 6.15 (s, 1H), 6.42 (s, 2H), 7.31-7.34 (d, 1H), 7.46 (s, 2H), 7.55 (bs, 2H), 8.21 (bs, 4H), 8.28-8.31 (d, 1H), 9.80 (d, 1H). |
| 255 | 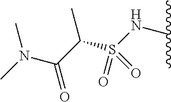 [SMILES: *NS(=O)(=O)[C@@H](C)C(=O)N(C)C] | 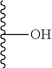 [SMILES: *O] | GS-3 | LC-MS: 510 [M + H]⁺. |
| 256 | 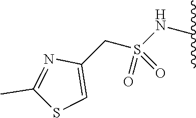 [SMILES: *NS(=O)(=O)Cc1csc(C)n1] | 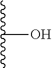 [SMILES: *O] | GS-3 | LC-MS: 522 [M + H]⁺. |
| 257 | 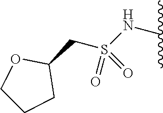 [SMILES: *NS(=O)(=O)C[C@H]1CCCO1] | 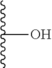 [SMILES: *O] | GS-3 | LC-MS: 495 [M + H]⁺. |
| 258 | 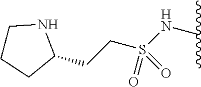 [SMILES: *NS(=O)(=O)CC[C@@H]1CCCN1] | 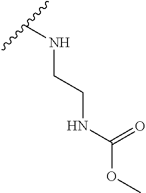 [SMILES: *NCCNC(=O)OC] | GS-1 | LC-MS: 608.4 [M + H]⁺; ¹HNMR (CD₃OD, 300 MHz): δ 1.52-1.62 (m, 1H), 1.92-2.10 (m, 3H), 2.13-2.22 (m, 3H), 3.11-3.20 (m, 4H), 3.25-3.29 (m, 2H), 3.56-3.34 (d, 2H), 3.56-3.62 (m, 3H), 4.34 (s, 2H), 6.46-6.48 (d, 3H), 7.25-7.27 (t, 2H), 7.57-7.58 (m, 3H), 7.83-7.85 (m, 1H), 8.22-8.24 (t, 2H). |
| 259 | 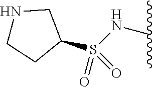 [SMILES: *NS(=O)(=O)[C@H]1CCNC1] | 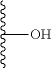 [SMILES: *O] | GS-1 | LC-MS: 480.3 [M + H]⁺; ¹HNMR (CD₃OD, 400 MHz): δ 2.17-2.22 (m, 1H), 2.24-2.30 (m, 1H), 3.28-3.31 (m, 1H), 3.32-3.34 (m, 1H), 3.47-3.59 (m, 3H), 3.81-3.87 (m, 1H), 4.33 (s, 2H), 6.22 (s, 1H), 6.53 (s, 2H), 7.51-7.59 (m, 4H), |
| | | | | 7.82-7.84 (m, 1H), 8.22-8.27 (m, 2H). |
| 260 | 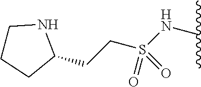 [SMILES: *NS(=O)(=O)CC[C@@H]1CCCN1] | 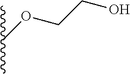 [SMILES: *OCCO] | GS-1 | LC-MS: 552.1 [M + H]⁺; ¹HNMR (CD₃OD, 400 MHz): δ 1.49-1.59 (m, 1H), 1.91-2.05 (m, 3H), 2.11-2.18 (m, 2H), 3.06-3.14 (m, 2H), 3.20-3.29 (m, 2H), 3.47-3.59 (m, 1H), 3.68-3.70 (t, 2H), 4.23-4.24 (t, 2H), 4.34 (s, 2H), 6.25 |
| | | | | (s, 1H), 6.53 (s, 2H), 7.30-7.31 (d, 1H), |
| | | | | 7.51 (s, 1H), 7.55-7.56 (m, 2H), 7.57- |
| | | | | 7.59 (m, 1H), 7.82-7.84 (m, 1H), 8.23- |
| | | | | 8.29 (m, 2H). |
| 261 | 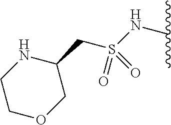 [SMILES: *NS(=O)(=O)C[C@H]1COCCN1] | 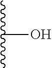 [SMILES: *O] | GS-2 | LC-MS: 508.2 [M + H]⁺; ¹HNMR (CD₃OD, 400 MHz): δ 3.16-3.23 (m, 2H), 3.30-3.34 (m, 2H), 3.34-3.55 (m, 1H), 3.55-3.75 (m, 2H), 3.75-4.33 (m, 2H), 4.86 (s, 2H), 6.27 (s, 1H), 6.51-6.52 (d, 2H), 7.26-7.28 (d, 1H), 7.44 (s, 1H), 7.53-7.56 (m, 3H), 7.82-7.84 (t, 1H), 8.20-8.24 (m, 2H). |
| 262 | 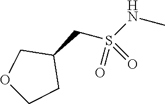 | 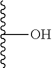 [SMILES: *O] | GS-3 | LC-MS: 495 [M + H]⁺. |
| 263 | 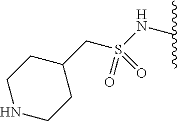 [SMILES: *NS(=O)(=O)CC1CCNCC1] | 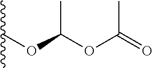 [SMILES: *O[C@@H](C)OC(C)=O] | GS-2 | LC-MS: 594.1 [M + H]⁺; ¹HNMR (CD₃OD, 400 MHz): δ 1.42-1.43 (d, 3H), 1.45-1.49 (bs, 2H), 1.52 (s, 3H), 2.03 (bs, 2H), 2.10-2.20 (m, 1H), 2.94-3.04 (m, 4H), 3.32-3.35 (m, 2H), 4.35 (s, 2H), 6.16 (s, 1H), 6.43-6.47 (d, 1H), 6.54-6.58 (d, 1H), 6.83-6.85 (dd, 1H), 7.30-7.32 (d, 1H), 7.50 (s, 1H), 7.57-7.59 (m, 2H), |
| | | | | 7.60-7.62 (m, 1H), 7.82-7.85 (dd, 1H), |
| | | | | 8.22-8.24 (d, 1H), 8.28-8.30 (d, 1H). |
| 264 | 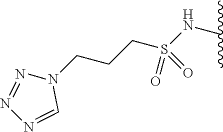 [SMILES: *NS(=O)(=O)CCCn1cnnn1] | 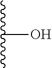 [SMILES: *O] | GS-3 | LC-MS: 521 [M + H]⁺. |
| 265 | 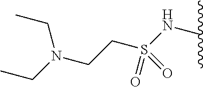 [SMILES: *NS(=O)(=O)CCN(CC)CC] | 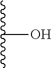 [SMILES: *O] | GS-3 | LC-MS: 510 [M + H]⁺. |
| 266 | 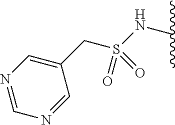 [SMILES: *NS(=O)(=O)Cc1cncnc1] | 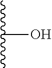 [SMILES: *O] | GS-2 | LC-MS: 503.0 [M + H]⁺; ¹HNMR (CD₃OD, 400 MHz): δ 4.36-4.40 (d, 4H), 6.15 (s, 1H), 6.53 (s, 2H) 7.28-7.30 (d, 1H), 7.49-7.51 (d, 2H), 7.55-7.7.57 (t, 2H), 7.80-7.82 (t, 1H), 8.21-8.27 (m, 2H), 8.45 (s, 2H), 8.96 (s, 1H). |
| 267 | 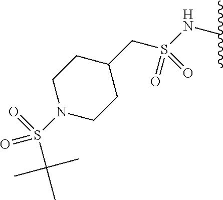 [SMILES: *NS(=O)(=O)CC1CCN(S(=O)(=O)C(C)(C)C)CC1] | 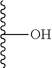 [SMILES: *O] | GS-1 | LC-MS: 628.4 [M + H]⁺; ¹HNMR (CD₃OD, 300 MHz): δ 1.13-1.17 (m, 2H), 1.32 (s, 9H), 1.70-1.74 (m, 2H), 1.81- 1.84 (m, 1H), 2.80-2.93 (m, 4H), 3.66- 3.70(m, 2H), 4.35 (s, 2H), 6.22 (s, 1H), 6.54 (s, 2H), 7.29-7.31 (d, 1H), 7.45 (s, 1H), 7.53-7.57 (m, 3H), 7.79-7.83 (m, 1H), 8.21-8.30 (m, 2H). |
| 268 | 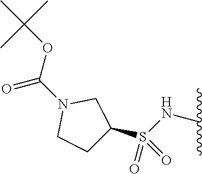 [SMILES: *NS(=O)(=O)[C@H]1CCN(C(=O)OC(C)(C)C)C1] | 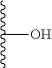 [SMILES: *O] | GS-1 | LC-MS: 580.4 [M + H]⁺; ¹HNMR (CD₃OD, 400 MHz): δ 1.38 (d, 9H), 2.14 (m, 2H), 3.29-3.30 (m, 1H), 3.65 (m, 4H), 4.34 (s, 2H), 6.25 (d, 1H), 6.53(s, 2H), 7.28 (d, 1H), 7.41 (s, 1H), 7.51- 7.57(m, 3H), 7.82 (d, 1H), 8.26-8.28 (m, 2H). |
| 269 | 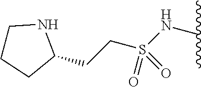 [SMILES: *NS(=O)(=O)CC[C@@H]1CCCN1] | 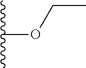 [SMILES: *OCC] | GS-1 | LC-MS: 535.6 [M + H]⁺; ¹HNMR (CD₃OD, 400 MHz): δ 1.13-1.17 (t, 3H), 1.53-1.61 (m, 1H), 1.93-2.10 (m, 5H), 3.10-3.13 (t, 2H), 3.20-3.35 (m, 2H), 3.56-3.61 (m, 1H), 3.78-4.21 (q, 2H), 4.35 (s, 2H), 6.26 (s, 1H), 6.53 (s, 2H), |
| | | | | 7.30-7.32 (d, 1H), 7.54-7.62 (m, 4H), |
| | | | | 7.84-7.86 (d, 1H), 8.24-8.29 (t, 2H). |
| 270 | 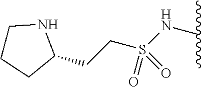 [SMILES: *NS(=O)(=O)CC[C@@H]1CCCN1] | 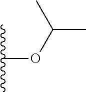 [SMILES: *OC(C)C] | GS-1 | LC-MS: 550.4 [M + H]⁺; ¹HNMR (CD₃OD, 400 MHz): δ 1.06-1.08 (d, 6H), 1.55-1.61 (m, 1H), 1.96-2.10 (m, 5H), 3.10-3.12 (t, 2H), 3.11-3.20 (m, 2H), 3.59-3.63 (m, 1H), 4.36 (s, 2H), 5.01- 5.07 (m, 1H), 6.24(s, 2H), 6.53 (s, 2H), |
| | | | | 7.30-7.32 (d, 1H), 7.53-7.65 (m, 3H), |
| | | | | 7.84-7.87 (d, 1H), 8.24-8.29 (m, 2H). |
| 271 | 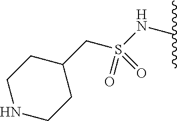 [SMILES: *NS(=O)(=O)CC1CCNCC1] | 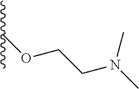 [SMILES: *OCCN(C)C] | GS-1 | LC-MS: 579.2 [M + H]⁺; ¹HNMR (CD₃OD, 400 MHz): δ 1.43-1.46 (m, 2H), 2.00-2.02 (m, 4H), 2.16-2.18 (m, 2H), 2.91-2.97 (m, 9H), 3.49-3.51 (m, 2H), 4.36 (s, 2H), 4.54-4.57 (m, 2H), 6.29 (s, 1H), 6.53 (s, 2H), 7.31-7.33 (d, 1H), 7.48 (s, 1H), 7.57-7.59 (m, 2H), 7.68 (s, 1H), 7.83 (m, 1H), 8.23-8.30 (m, 2H). |
| 272 | 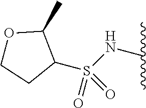 [SMILES: *NS(=O)(=O)C1CCO[C@H]1C] | 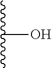 [SMILES: *O] | GS-3 | LC-MS: 495 [M + H]⁺. |
| 273 | 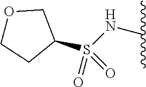 [SMILES: *NS(=O)(=O)[C@H]1CCOC1] | 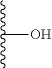 [SMILES: *O] | GS-3 | LC-MS: 481 [M + H]⁺. |
| 274 | 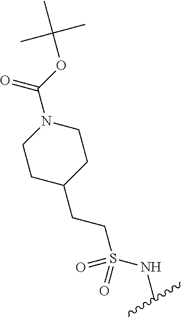 [SMILES: *NS(=O)(=O)CCC1CCN(C(=O)OC(C)(C)C)CC1] | 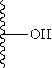 [SMILES: *O] | GS-1 | LC-MS: 622.5 [M + H]⁺; ¹HNMR (CD₃OD, 400 MHz): δ 0.82-0.91 (m, 2H), 1.40 (s, 9H), 1.47-1.52 (d, 4H), 2.01 (s, 1H), 2.60 (bs, 2H), 2.89-2.93 (t, 2H), 3.92-3.95 (d, 2H), 4.30 (s, 2H), 6.25 (s, 1H), 6.52 (s, 2H), 7.23-7.25 (d, 1H), 7.44-7.45 (d, 2H), 7.52-7.55 (d, 2H), 7.78-7.80 (d, 1H), 8.20-8.23 (m, 2H). |
| 275 | 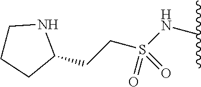 [SMILES: *NS(=O)(=O)CC[C@@H]1CCCN1] | 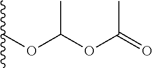 [SMILES: *OC(C)OC(C)=O] | GS-2 | LC-MS: 594.1 [M + H]⁺; ¹HNMR (CD₃OD, 400 MHz): δ 1.44-1.45(dd, 3H), 1.54-1.63 (m, 3H), 1.92-2.08 (m, 4H), 2.12-2.23(m, 2H), 3.08-3.21 (m, 2H), 3.25-3.30 (m, 2H), 3.57-3.61 (m, 1H), 4.35 (s, 2H), 6.18-6.19 (d, 1H), 6.45-6.50 |
| | | | | (d, 1H), 6.56-6.58 (d, 1H), 6.83-6.88 (m, |
| | | | | 1H), 7.31-7.32 (d, 1H), 7.48-7.52 (m, |
| | | | | 1H), 7.56-7.62 (m, 3H), 7.83-7.85 (t, |
| | | | | 1H), 8.23-8.24 (d, 1H), 8.29-8.31 (d, |
| | | | | 1H). |
| 276 | 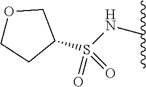 [SMILES: *NS(=O)(=O)[C@@H]1CCOC1] | 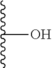 [SMILES: *O] | GS-3 | LC-MS: 481 [M + H]⁺. |
| 277 | 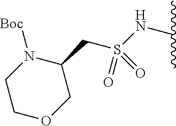 [SMILES: *NS(=O)(=O)C[C@H]1COCCN1C(=O)OC(C)(C)C] | 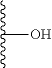 [SMILES: *O] | GS-1 | LC-MS: 610.1 [M + H]⁺; ¹HNMR (CD₃OD, 400 MHz): δ 1.34 (bs, 9H), 2.54-2.61 (m, 1H), 3.14-3.18 (m, 2H), 3.38-3.46 (m, 2H), 3.61-3.76 (m, 3H), 4.32 (s, 3H), 6.23 (bs, 1H), 6.52 (s, 2H), 7.25-7.27 (d, 1H), 7.49-7.57 (m, 4H), 7.80-7.82 (t, 1H), 8.20-8.25 (m, 2H) |
| 278 | 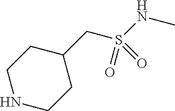 | 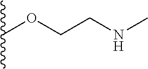 [SMILES: *OCCNC] | GS-2 | LC-MS: 565.4 [M + H]⁺; ¹HNMR(CD₃OD, 400 MHz): δ 1.42-1.48 (m, 2H), 1.94-1.97 (bs, 2H), 2.11-2.12 (m, 1H), 2.74 (s, 3H), 2.87-2.94 (m, 4H), 3.33-3.36 (m, 4H), 4.35 (s, 2H), 4.45- 4.48 (m, 2H), 6.28 (s, 1H), 6.52 (s, 2H), 7.30-7.32 (d, 1H), 7.49 (s, 1H), 7.55-7.58 |
| | | | | (m, 2H), 7.70 (s, 1H), 7.81-7.84 (m, 1H), |
| | | | | 8.22-8.26 (m, 1H), 8.28 (s, 1H). |
| 279 | 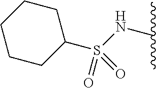 [SMILES: *NS(=O)(=O)C1CCCCC1] | 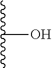 [SMILES: *O] | GS-3 | LC-MS: 493[M + H]⁺. |
| 280 | 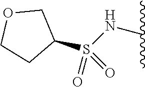 [SMILES: *NS(=O)(=O)[C@H]1CCOC1] | 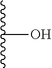 [SMILES: *O] | GS-3 | LC-MS: 481[M + H]⁺. |
| 281 | 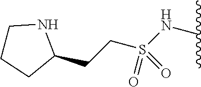 [SMILES: *NS(=O)(=O)CC[C@H]1CCCN1] | 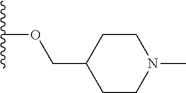 [SMILES: *OCC1CCN(C)CC1] | GS-2 | LC-MS: 618.8 [M + H]⁺; ¹HNMR(CD₃OD, 400 MHz): δ 1.39-1.59 (m, 3H), 1.73-1.80 (m, 3H), 1.92-2.06 (m, 3H), 2.09-2.19 (m, 2H), 2.72-2.76 (m, 1H), 2.79 (s, 3H), 3.10-3.14 (m, 3H), 3.23-3.25 (m, 2H), 3.39-3.42 (m, 2H), |
| | | | | 3.55-3.59 (m, 1H), 4.08-4.10 (d, 2H), |
| | | | | 4.34 (s, 2H), 6.24 (s, 1H), 6.50 (s, 2H), |
| | | | | 7.29-7.31 (d, 1H), 7.52 (s, 1H), 7.55-7.59 |
| | | | | (m, 3H), 7.83-7.85 (m, 1H), 8.21-8.23 |
| | | | | (m, 1H), 8.25-8.27 (d, 1H). |
| 282 | 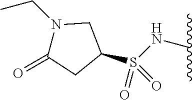 [SMILES: *NS(=O)(=O)[C@H]1CC(=O)N(CC)C1] | 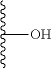 [SMILES: *O] | GS-3 | LC-MS: 522 [M + H]⁺. |
| 283 | 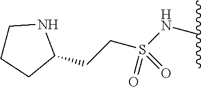 [SMILES: *NS(=O)(=O)CC[C@@H]1CCCN1] | 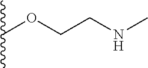 [SMILES: *OCCNC] | GS-1 | LC-MS: 565.1 [M + H]⁺; ¹HNMR(CD₃OD, 400 MHz): δ 1.55-1.56 (m, 1H), 1.95-2.25 (m, 5H), 2.75 (s, 3H), 3.11-3.12 (m, 2H), 3.24-3.27 (m, 2H), 3.34-3.37 (m, 2H), 3.6 (m, 1H), 4.35 (s, 2H), 4.46-4.49 (m, 2H), 6.29 (s, 1H), |
| | | | | 6.53 (s, 2H), 7.30-7.33 (d, 1H), 7.512- |
| | | | | 7.516 (d, 1H), 7.56-7.59 (m, 2H), 7.69 (s, |
| | | | | 1H), 7.83 (d, 1H), 8.27-8.28 (d, 1H), |
| | | | | 8.29-8.30 (d, 1H). |
| 284 | 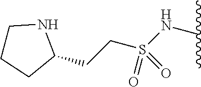 [SMILES: *NS(=O)(=O)CC[C@@H]1CCCN1] | 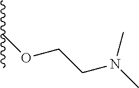 [SMILES: *OCCN(C)C] | GS-1 | LC-MS: 579.4 [M + H]⁺; ¹HNMR(CD₃OD, 400 MHz): δ 1.50-1.51 (m, 1H), 1.96-2.18 (m, 4H), 2.71-2.72 (d, 1H), 2.94-3.09 (d, 6H), 3.10-3.14 (m, 2H), 3.23-3.29 (m, 2H), 3.30-3.57 (m, 3H), 4.35 (s, 2H), 4.54-4.57 (m, 2H), |
| | | | | 6.29-6.30 (d, 1H), 6.53 (s, 2H), 7.30-7.32 |
| | | | | (d, 1H), 7.514-7.519 (d, 1H), 7.57-7.59 |
| | | | | (m, 2H), 7.68 (s, 1H), 7.83-7.85 (m, 1H), |
| | | | | 8.24-8.29 (d, 2H). |

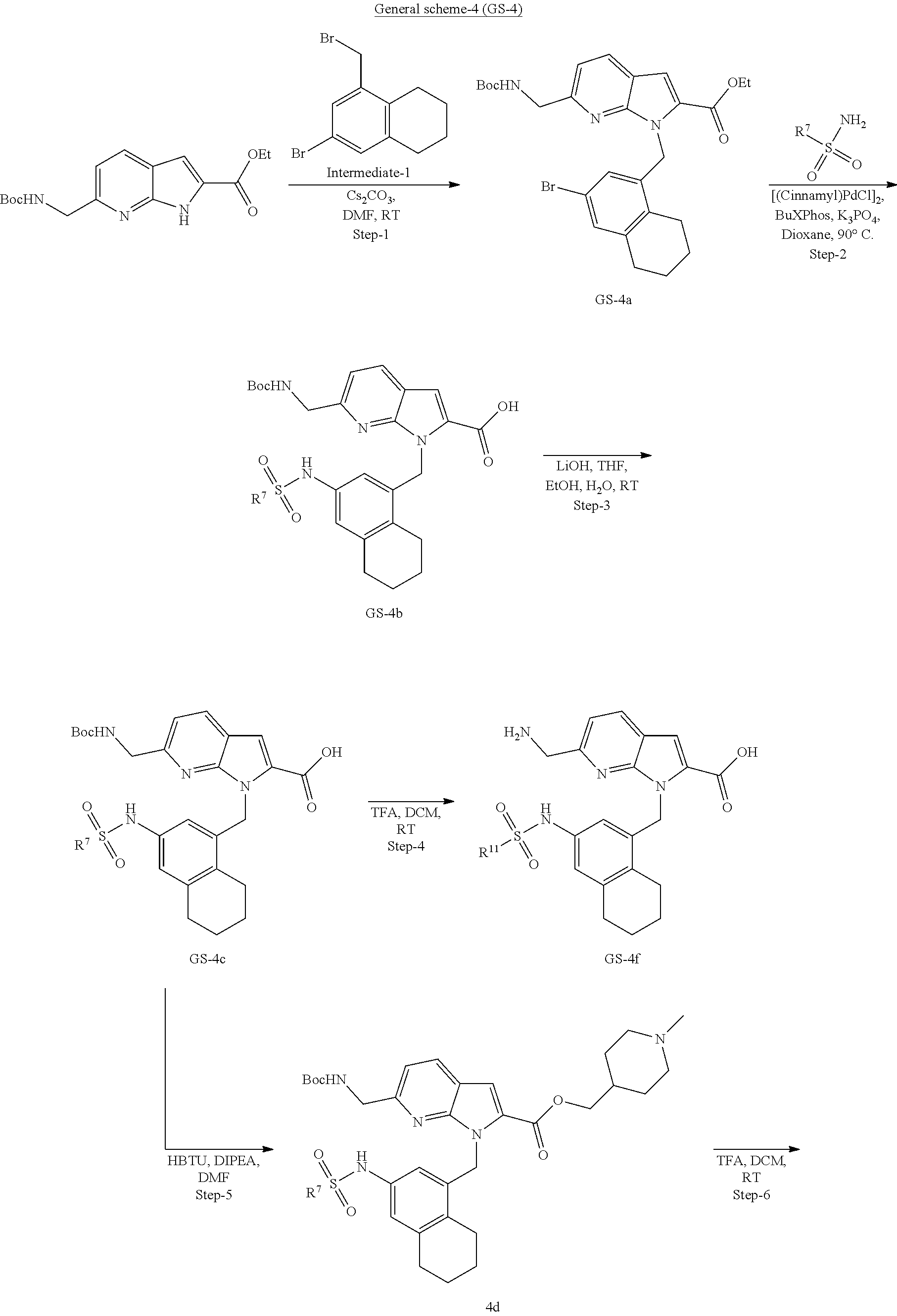

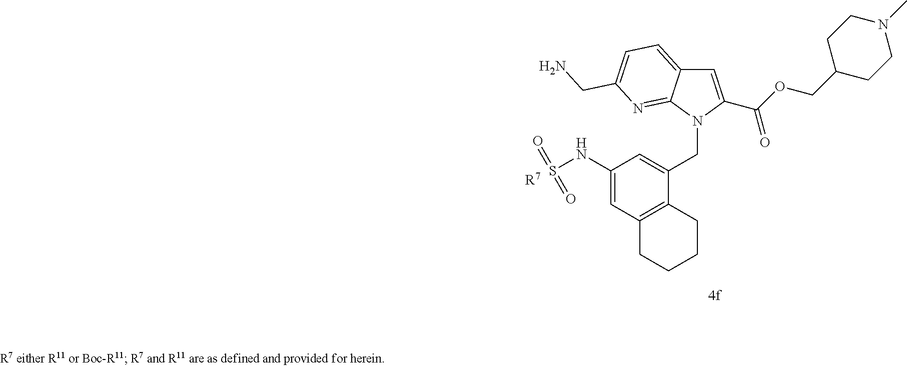

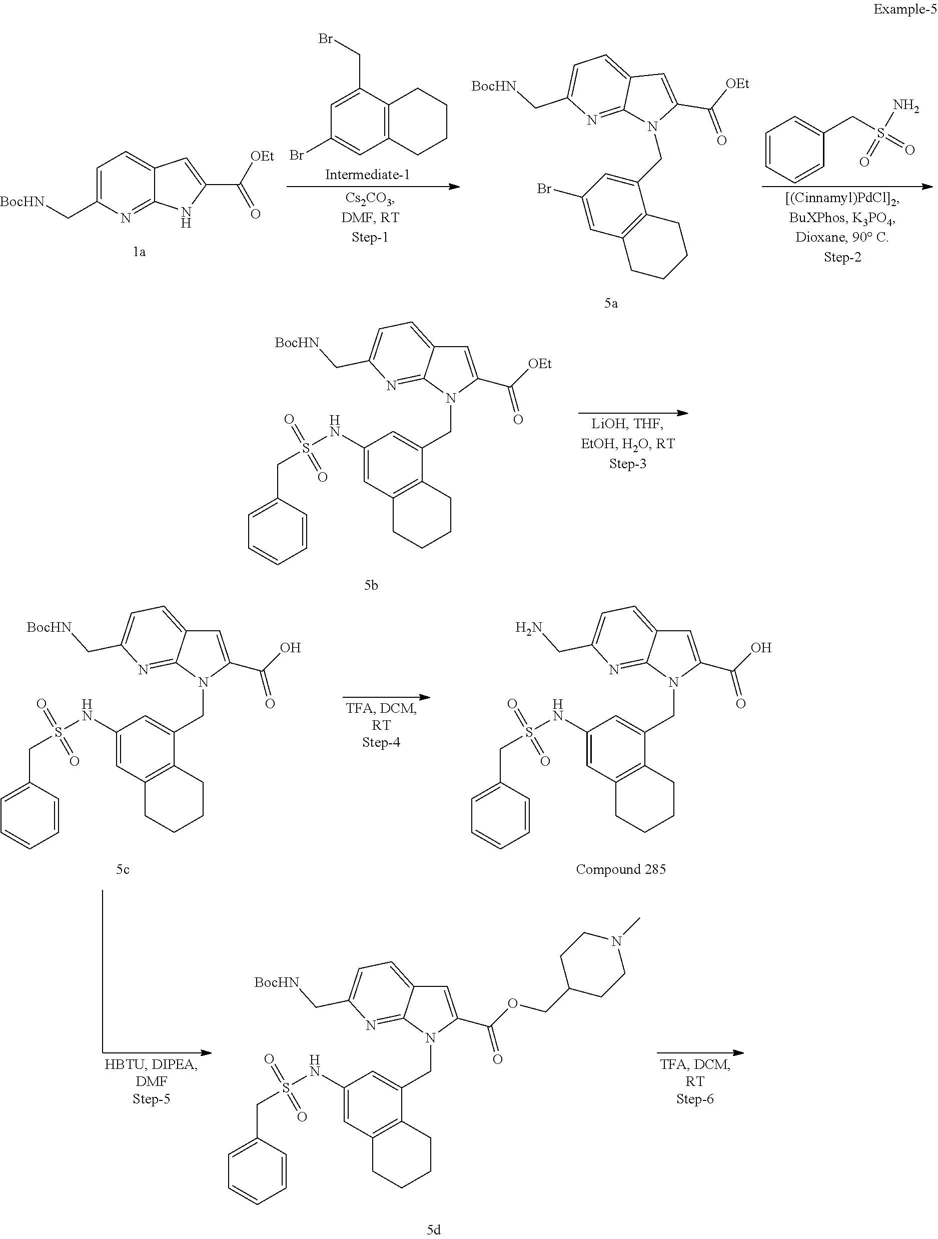

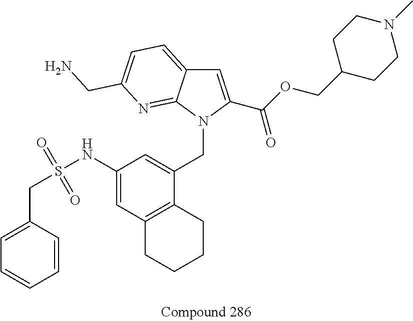 [SMILES: CN1CCC(COC(=O)c2cc3ccc(CN)nc3n2Cc2cc(NS(=O)(=O)Cc3ccccc3)cc3c2CCCC3)CC1]

### Step-1: Synthesis of ethyl 1-((3-bromo-5,6,7,8-tetrahydronaphthalen-1-yl)methyl)-6-(((tert-butoxycarbonyl)amino)methyl)-1H-pyrrolo[2,3-b]pyridine-2-carboxylate

To a stirred solution of ethyl 6-(((tert-butoxycarbonyl)amino)methyl)-1H-pyrrolo[2,3-b]pyridine-2-carboxylate (1.2 g, 3.75 mmol) in DMF (12 mL) was added Cs₂CO₃ (0.06 g, 9.3 mmol). After stirring for 10 minutes at RT, 7-bromo-5-(bromomethyl)-1,2,3,4-tetrahydronaphthalene (1.1 g, 3.75 mmol) was added and stirred for 6 hours at RT. The reaction mixture was added ice-cold water to form a solid that was filtered and dried. The crude solid was purified by Combi flash chromatography using 20% ethyl acetate in hexane as eluent to afford pure title compound (1.7 g, 83.3%). LC-MS: 543.8 [M+H]⁺.

### Step-2: Synthesis of ethyl 6-(((tert-butoxycarbonyl)amino)methyl)-1-((3-((phenylmethyl)sulfonamido)-5,6,7,8-tetrahydronaphthalen-1-yl)methyl)-1H-pyrrolo[2,3-b]pyridine-2-carboxylate

To a stirred solution of ethyl 1-((3-bromo-5,6,7,8-tetrahydronaphthalen-1-yl)methyl)-6-(((tert-butoxycarbonyl)amino)methyl)-1H-pyrrolo[2,3-b]pyridine-2-carboxylate (0.6 g, 1.1 mmol) in 1,4-dioxane (6 mL) was added phenylmethanesulfonamide (0.28 g, 1.65 mmol) and K₃PO₄ (0.157 g, 0.74 mmol) at RT. The resultant mixture was degassed with nitrogen and to the mixture was added tert butyl X-Phos (0.055 g, 0.11 mmol) followed by [Pd(cinnamyl chloride)]2 (0.022 g, 0.055 mmol). The reaction mixture was then stirred for at 90° C. for 12 h. After cooling to RT, the mixture was filtered through Celite. and the filtrate was concentrated. The crude was purified by Combi flash chromatography using 20% ethyl acetate in hexane to afford the pure title compound (0.17 g, 24.2%). LC-MS: 633.10 [M+H]⁺.

### Step-3: Synthesis of 6-(((tert-butoxycarbonyl)amino)methyl)-1-((3-((phenylmethyl)sulfonamido)-5,6,7,8-tetrahydronaphthalen-1-yl)methyl)-1H-pyrrolo[2,3-b]pyridine-2-carboxylic acid

A stirred solution of ethyl 6-(((tert-butoxycarbonyl)amino)methyl)-1-((3-((phenylmethyl)sulfonamido)-5,6,7,8-tetrahydronaphthalen-1-yl)methyl)-1H-pyrrolo[2,3-b]pyridine-2-carboxylate (0.17 g, 0.26 mmol) in THF (3 mL), ethanol (3 mL) was added LiOH monohydrate (0.032 g, 1.34 mmol) in water (3 mL). The reaction mixture was stirred at RT for 3 h, concentrated, acidified with citric acid, extracted with 10% MeOH: in DCM. Organic portion was dried Na₂SO₄ and concentrated to get title compound (0.145 g, 89.1%). LC-MS: 605.05 [M+H]⁺.

### Step-4: Synthesis of 6-(aminomethyl)-1-((3-((phenylmethyl)sulfonamido)-5,6,7,8-tetrahydronaphthalen-1-yl)methyl)-1H-pyrrolo[2,3-b]pyridine-2-carboxylic acid

A solution of 6-(((tert-butoxy carbonyl)amino)methyl)-1-((3-((phenylmethyl)sulfonamido)-5,6,7,8-tetrahydronaphthalen-1-yl)methyl)-1H-pyrrolo[2,3-b]pyridine-2-carboxylic acid (0.2 g, 0.33 mmol) in DCM (5 mL) was cooled to 0° C. and to the solution was added TFA (1.6 mL). After addition, the reaction mixture was stirred at RT for 12 h. The reaction mixture was then concentrated and washed with diethyl ether, followed by washing with pentane and dried to get the pure title compound (0.085 g, 50.8%). LC-MS: 505.3 [M+H]⁺; ¹HNMR (CD₃OD, 400 MHz): δ 1.83-1.85 (m, 2H), 1.95-1.97 (m, 2H), 2.75-2.78 (m, 2H), 2.88-2.91 (m, 2H), 4.086 (s, 2H), 4.34-4.35 (m, 2H), 5.84-5.844 (d, 1H), 6.010 (s, 2H), 6.701-6.706 (d, 1H), 7.07-7.09 (m, 2H), 7.25-7.33 (m, 4H), 7.41 (s, 1H), 8.22-8.24 (d, 1H).

### Step-5: Synthesis of (1-methylpiperidin-4-yl)methyl 6-(((tert-butoxycarbonyl)amino)methyl)-1-((3-((phenylmethyl)sulfonamido)-5,6,7,8-tetrahydronaphthalen-1-yl)methyl)-1H-pyrrolo[2,3-b]pyridine-2-carboxylate

To a stirred solution of 6-(((tert-butoxycarbonyl)amino)methyl)-1-((3-((phenylmethyl)sulfonamido)-5,6,7,8-tetrahydronaphthalen-1-yl)methyl)-1H-pyrrolo[2,3-b]pyridine-2-carboxylic acid (0.145 g, 0.24 mmol) in DMF was added HBTU (0.112 g, 0.48 mmol), DIPEA (0.124 g, 0.96 mmol) after stirring at RT for 15 minutes, added (1-methylpiperidin-4-yl)methanol (0.124 g, 0.96 mmol) and stirred for overnight at RT. The reaction mixture wad added into ice-cold water, solid separated was filtered and dried (0.16 g. crude). This was used as such in the next step. LC-MS: 716.3 [M+H]⁺.

### Step-6: Synthesis of (1-methylpiperidin-4-yl)methyl 6-(aminomethyl)-1-((3-((phenylmethyl)sulfonamido)-5,6,7,8-tetrahydronaphthalen-1-yl)methyl)-1H-pyrrolo[2,3-b]pyridine-2-carboxylate

A solution of (1-methylpiperidin-4-yl)methyl 6-(((tert-butoxycarbonyl)amino)methyl)-1-((3-((phenylmethyl)sulfonamido)-5,6,7,8-tetrahydronaphthalen-1-yl)methyl)-1H-pyrrolo[2,3-b]pyridine-2-carboxylate (0.16 g, 0.22 mmol) in DCM (5 mL) was cooled to 0° C., added TFA (1.5 mL) and stirred for overnight at RT. The reaction mixture was then concentrated and the residue was purified by preparative HPLC to afford pure title compound (0.025 g, 18.2%). LC-MS: 616.5 [M+H]⁺; HNMR (CD₃OD, 400 MHz): δ 1.41-1.62 (m, 2H), 1.83-1.99 (m, 8H), 2.77-2.80 (t, 2H), 2.854-2.983 (m, 4H), 3.45-3.48 (d, 4H), 4.05 (s, 2H), 4.17-4.18 (d, 2H), 4.39-4.40 (m, 2H), 5.872-5.877 (d, 1H), 6.00 (s, 2H), 6.65-6.655 (m, 1H), 7.05-7.07 (m, 2H), 7.25-7.34 (m, 4H), 7.57 (s, 1H), 8.29-8.31 (d, 1H).

The following compounds listed in Table 2 were prepared according to GS-4 by following the similar procedures as described above for Example-5 using appropriate reagents with suitable modifications known to the one skilled in the art.

TABLE 2

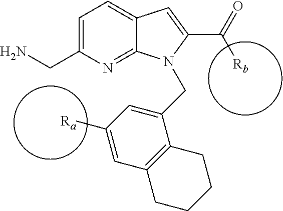

| Cpmd # | Rₐ | R_{b} | Scheme | Spectral data |
|---|---|---|---|---|
| 287 | 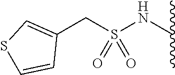 [SMILES: *NS(=O)(=O)Cc1ccsc1] | 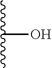 [SMILES: *O] | GS-4 | LC-MS: 511.3 [M + H]⁺; ¹HNMR(CD₃OD, 400 MHz): δ 1.83-1.88 (m, 2H), 1.95-1.98 (m, 2H), 2.78-2.80 (t, 2H), 2.88-2.91 (m, 2H), 4.14 (s, 2H), 4.38 (s, 2H), 5.74-5.77 (d, 1H), 5.98 (s, 2H), 6.74-6.75 |
| | | | | (d, 1H), 6.84-6.87 (d, 1H), 7.03-7.04 (m, 1H), |
| | | | | 7.28 (s, 1H), 7.31-7.33 (m, 1H), 7.50 (s, 1H), 8.25- |
| | | | | 8.28 (d, 1H). |

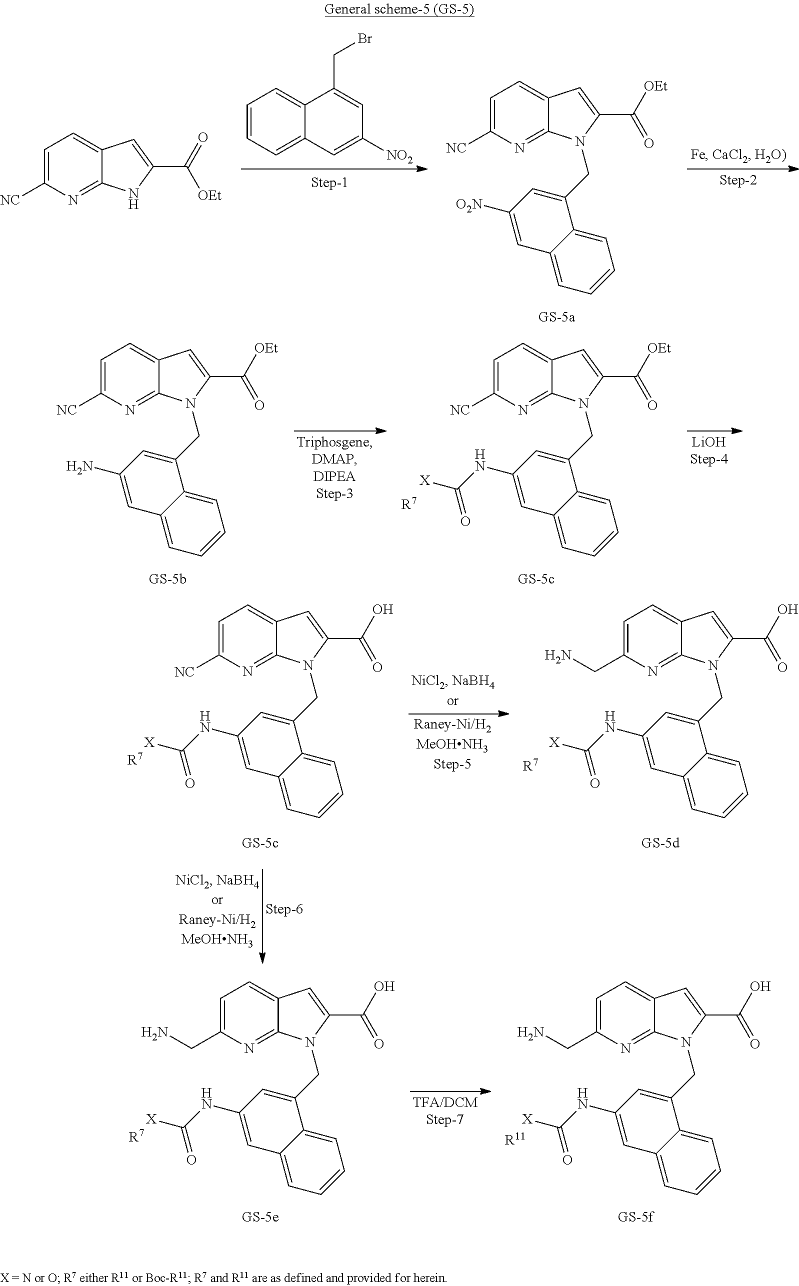

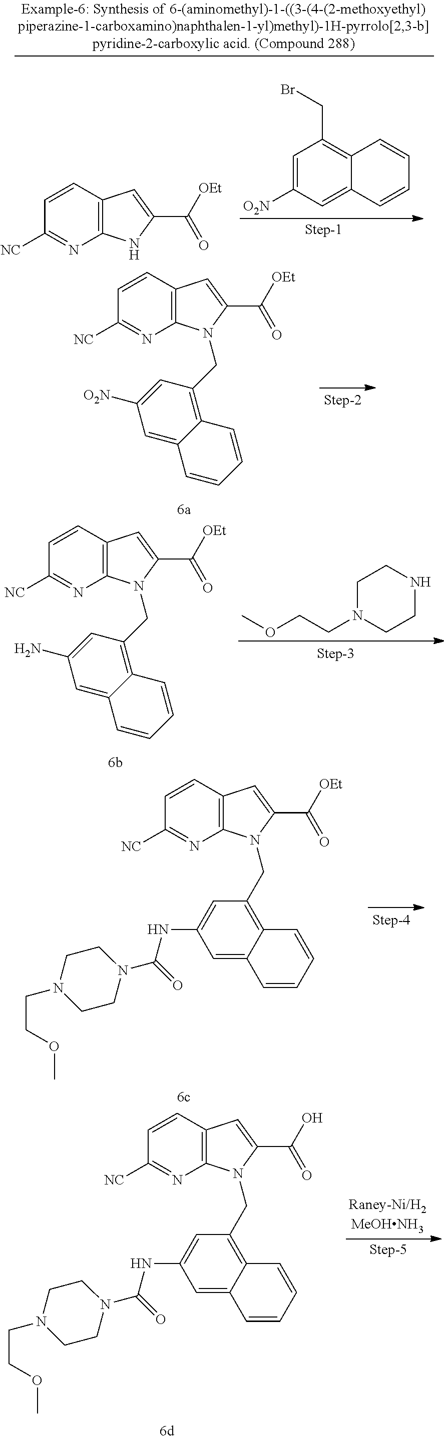

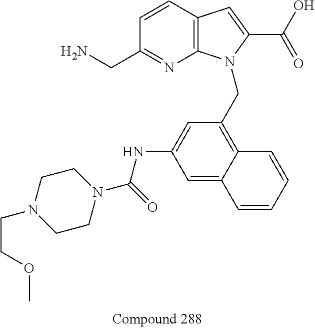 [SMILES: COCCN1CCN(C(=O)Nc2cc(Cn3c(C(=O)O)cc4ccc(CN)nc43)c3ccccc3c2)CC1]

### Step-1: Synthesis of ethyl 6-cyano-1-(3-nitronaphthalen-1-yl)methyl)-1H-pyrrolo[2,3-b]pyridine-2-carboxylate

To a stirred solution of ethyl 6-cyano-1H-pyrrolo[2,3-b]pyridine-2-carboxylate (1 g, 3.75 mmol) in DMF (20 mL) was added Cs₂CO₃ (3.67 g, 11.2 mmol) followed by 1-(bromomethyl)-3-nitronaphthalene (0.8 g, 3.75 mmol) and stirred at RT for 16 h at RT. The reaction mixture was then poured into ice-cold water, and the resulting solids were filtered and dried. This was purified by Combi flash column chromatography using DCM as eluent. This afforded the pure title compound 6a (0.9 g, 48.3%). LC-MS: 401.3 [M+H]⁺.

### Step-2: Synthesis of ethyl 1-((3-aminonaphthalen-1-yl)methyl)-6-cyano-1H-pyrrolo[2,3-b]pyridine-2-carboxylate

Ethyl 6-cyano-1-((3-nitronaphthalen-1-yl)methyl)-1H-pyrrolo[2,3-b]pyridine-2-carboxylate (2 g, 4.99 mmol) was dissolved in 1,4-dioxane (20 mL), ethanol (20 mL), water (1.8 mL) was added Fe powder (2.52 g, 45 mmol) and CaCl2·2H₂O (2.13 g, 15 mmol). The resulting mixture was stirred at 80° C. for 16 h. The reaction mixture was filtered and washed with ethyl acetate, and the filtrate was then washed with water, dried over Na₂SO₄ and concentrated to yield the title compound 6b (1.7 g, 91.8%). LC-MS: 371.20 [M+H]⁺.

### Step-3: Synthesis of ethyl 6-cyano-1-((3-(4-(2-methoxyethyl)piperazine-1-carboxamido)naphthalen-1-yl)methyl)-1H-pyrrolo[2,3-b]pyridine-2-carboxylate

To a stirred solution of triphosgene (0.016 g, 0.053 mmol) in THF (5 mL) was cooled to 0° C. and to the solution were added ethyl 1-((3-aminonaphthalen-1-yl)methyl)-6-cyano-1H-pyrrolo[2,3-b]pyridine-2-carboxylate (0.05 g, 0.134 mmol) and DIPEA (0.104 g, 0.809 mmol). The mixture was stirred for 30 minutes, then 1-(2-methoxyethyl)piperazine (0.021 g, 0.148 mmol) and DMAP (0.098 g, 0.08 mmol) were added at RT. The reaction mixture was quenched with methanol, concentrated to yield the title compound 6c (0.12 g. crude) this was used as such in the next step. LC-MS: 541.4 [M+H]⁺.

### Step-4: Synthesis of 6-cyano-1-((3-(4-(2-methoxyethyl)piperazine-1-carboxamido)naphthalen-1-yl)methyl)-1H-pyrrolo[2,3-b]pyridine-2-carboxylic acid

To a solution of ethyl 6-cyano-1-((3-(4-(2-methoxyethyl)piperazine-1-carboxamido)naphthalen-1-yl)methyl)-1H-pyrrolo[2,3-b]pyridine-2-carboxylate (0.35 g, 0.64 mmol) in THF (10 mL), ethanol (1 mL) was added LiOH·H2O (0.108 g, 2.5 mmol) in water (5 mL) and the mixture stirred for 3 h at RT. The reaction mixture was concentrated, added water and acidified. The resulting solid was filtered and dried to yield the title compound 6d (0.32 g, crude), used as was in the next step. LC-MS: 513.30 [M+H]⁺.

### Step-5: Synthesis of 6-(aminomethyl)-1-((3-(4-(2-methoxyethyl)piperazine-1-carboxamido)naphthalen-1-yl)methyl)-1H-pyrrolo[2,3-b]pyridine-2-carboxylic acid. (Compound 288)

To a stirred solution of 6-cyano-1-((3-(4-(2-methoxyethyl)piperazine-1-carboxamido)naphthalen-1-yl)methyl)-1H-pyrrolo[2,3-b]pyridine-2-carboxylic acid (0.22 g, 0.42 mmol) in methanolic ammonia (20 mL) was added Raney nickel (0.4 g) and stirred under the positive pressure of hydrogen using a bladder. After 3 h. the reaction mixture was filtered through Celite bed and the filtrate was concentrated. The residue was purified by preparative HPLC to afford the pure title compound (Compound 288) (0.13 g, 58.8%). (Prep HPLC method: Column-Gemini NX (250 mm×21 mm), flow rate-15 mL/min, mobile phase-A was 0.1% TFA in water, B was acetonitrile, Gradient program-10% B at 0 minute, 20% B at 2 minutes, 50% B at 8 minutes). LC-MS: 517.3 [M+H]⁺; ¹HNMR (CD₃OD, 400 MHz): δ 2.97-3.16 (bs, 4H), 3.35-3.37 (m, 2H), 3.40 (s, 3H), 3.70-3.72 (m, 2H), 3.64-3.95 (m, 4H), 4.33 (s, 2H), 6.40 (s, 1H), 6.55 (s, 2H), 7.25-7.27 (m, 1H), 7.49-7.53 (m, 3H), 7.73 (s, 1H), 7.79-7.80 (m, 1H), 8.19-8.23 (m, 2H).

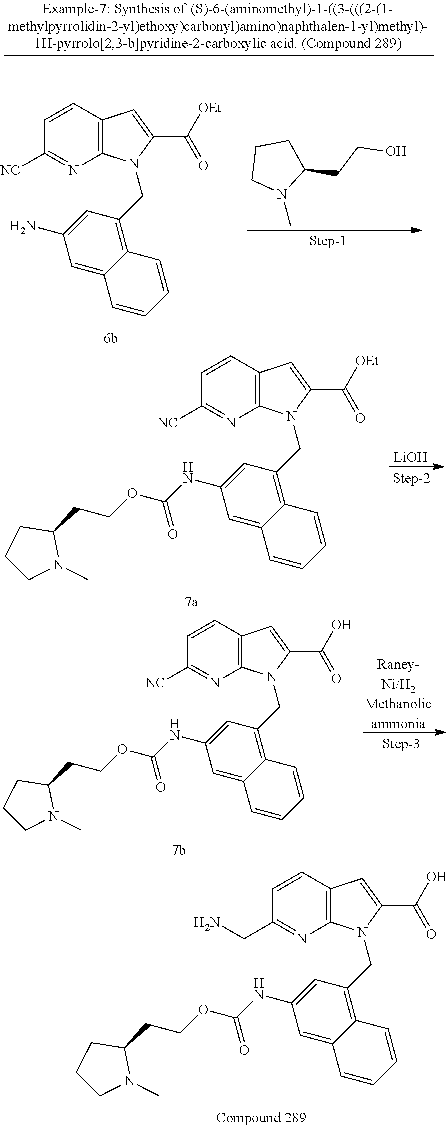

### Step-1: Synthesis of ethyl 1-((3-aminonaphthalen-1-yl)methyl)-6-cyano-1H-pyrrolo[2,3-b]pyridine-2-carboxylate

An ice-cold solution of triphosgene (0.126 g, 0.42 mmol) THF (35 mL) was added DIPEA (0.98 g, 5.66 mmol) followed by ethyl 1-((3-aminonaphthalen-1-yl)methyl)-6-cyano-1H-pyrrolo[2,3-b]pyridine-2-carboxylate (0.35 g, 0.94 mmol) in THF (10 mL). The reaction mixture was stirred at RT in 45 minutes. Then added(S)-2-(1-methylpyrrolidin-2-yl) ethan-1-ol (0.219 g, 1.7 mmol) in THF (8 mL) and DMAP (0.069 g, 0.56 mmol). The resultant mixture was stirred for 2.5 h and concentrated. The crude compound was purified by flash column chromatography using 10% methanol in DCM to get the pure title compound 7a (0.245 g, 49.3%). LC-MS: 526.2 [M+H]⁺.

### Step-2: Synthesis of(S)-6-cyano-1-(3-(((2-1-methylpyrrolidin-2-yl)ethoxy)carbonyl)amino)naphthalen-1-yl)methyl)-1H-pyrrolo[2,3-b]pyridine-2-carboxylic acid

A stirred mixture of ethyl 1-((3-aminonaphthalen-1-yl)methyl)-6-cyano-1H-pyrrolo[2,3-b]pyridine-2-carboxylate (0.245 g, 0.466 mmol), THF (3 mL) and ethanol (2 mL) was added LiOH·H2O (0.078 g, 1.86 mmol) in water (2 mL) at RT. The reaction mixture was stirred for 3 h and concentrated. The residue was dissolved in ice-cold water, acidified with 1N HCl to get solid. This was dissolved in DCM, dried over Na₂SO₄ and concentrated to yield the title compound 7b (0.21 g, crude), this was used as such in the next step. LC-MS: 498.3 [M+H]⁺.

### Step-3: (S)-6-(aminomethyl)-1-((3-(((2-(1-methylpyrrolidin-2-yl)ethoxy)carbonyl)amino) naphthalen-1-yl)methyl)-1H-pyrrolo[2,3-b]pyridine-2-carboxylic acid. (compound 289)

To a solution of(S)-6-cyano-1-((3-(((2-(1-methylpyrrolidin-2-yl)ethoxy)carbonyl) amino)naphthalen-1-yl)methyl)-1H-pyrrolo[2,3-b]pyridine-2-carboxylic acid (0.21 g, 0.422 mmol) in 7% methanolic ammonia (5 mL) was added Raney nickel (0.45 g) and stirred under positive pressure of hydrogen. After 2 h, reaction mixture was filtered through a pad of Celite, and the filtrate was concentrated. The residue was purified by preparative HPLC to afford the pure title compound 7c (0.07 g, 33.0%). (Prep HPLC method: Column-ZORBAX (150 mm×21 mm), flow rate-20 mL/min, mobile phase-A was 0.1% TFA in water, B was 1:1 acetonitrile & methanol, Gradient program-10% B at 0 minute, 20% B at 4 minutes, 50% B at 8 minutes). LC-MS: 502.40 [M+H]⁺; ¹HNMR (CD₃OD, 300 MHz): δ 1.65-1.83 (m, 2H), 1.92-2.04 (m, 2H), 2.06-2.31 (m, 2H), 2.83 (s, 3H), 3.07-3.10 (m, 2H), 3.56-3.59 (m, 1H), 4.09-4.13 (t, 2H), 4.30 (s, 2H), 6.30 (s, 1H), 6.54 (s, 2H), 7.23-7.25 (d, 1H), 7.41 (s, 1H), 7.49-7.52 (m, 2H), 7.76-7.79 (m, 1H), 7.89 (s, 1H), 8.18-8.23 (m, 2H).

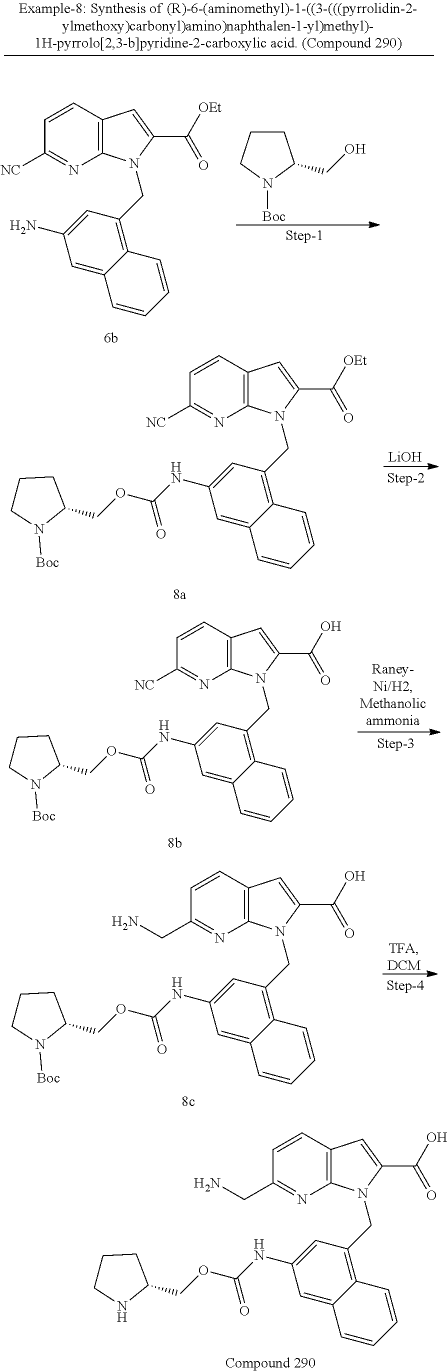

### Step-1: Synthesis of ethyl (R)-1-((3-((((1-(tert-butoxycarbonyl)pyrrolidin-2-yl)methoxy)carbonyl)amino)naphthalen-1-yl)methyl)-6-cyano-1H-pyrrolo[2,3-b]pyridine-2-carboxylate

The experimental procedure was the same as described in Step-3 of Example-6; appropriate variations in coupling methods, reactants, quantities of reagents, and solvents were used to obtain the compound 8a (0.35 g, crude). LC-MS: 596.0 [M+H]⁺.

### Step-2: Synthesis of (R)-1-((3-((((1-(tert-butoxycarbonyl)pyrrolidin-2-yl)methoxy)carbonyl)amino)naphthalen-1-yl)methyl)-6-cyano-1H-pyrrolo[2,3-b]pyridine-2-carboxylic acid

The experimental procedure was the same as described in Step-4 of Example-6; appropriate variations in coupling methods, reactants, quantities of reagents, and solvents were used to obtain the compound 8b (0.35 g, crude). LC-MS: 568.30 [M−H].

### Step-3: Synthesis of (R)-6-(aminomethyl)-1-((3-((((1-(tert-butoxycarbonyl)pyrrolidin-2-yl)methoxy)carbonyl)amino)naphthalen-1-yl)methyl)-1H-pyrrolo[2,3-b]pyridine-2-carboxylic acid

The experimental procedure was the same as described in Step-S of Example-6 with appropriate variations in coupling methods, reactants, quantities of reagents, and solvents to obtain the compound 8c (0.32 g, crude). LC-MS: 574.2 [M+H]⁺.

### Step-4: Synthesis of (R)-6-(aminomethyl)-1-((3-(((pyrrolidin-2-ylmethoxy)carbonyl)amino)naphthalen-1-yl)methyl)-1H-pyrrolo[2,3-b]pyridine-2-carboxylic acid

A stirred solution of (R)-6-(aminomethyl)-1-((3-((((1-(tert-butoxycarbonyl)pyrrolidin-2-yl)methoxy)carbonyl)amino)naphthalen-1-yl)methyl)-1H-pyrrolo[2,3-b]pyridine-2-carboxylic acid (0.32 g, 0.558 mmol) in DCM (5 mL) was added TFA (0.4 mL) and stirred at RT for 4 h. The reaction mixture was concentrated and purified by preparative HPLC to get the pure title compound (Compound 290) (0.1 g, 37.8%). (Prep HPLC method: Column-ZORBAX (150 mm×21 mm), flow rate-20 mL/min, mobile phase-A was 0.1% TFA in water, B was acetonitrile, Gradient program-20% B at 0 minute, 30% B at 4 minutes, 60% B at 8 minutes). LC-MS: 474.3 [M+H]⁺; ¹HNMR (CD₃OD, 400 MHz): δ 1.74-1.79 (m, 1H), 1.97-2.19 (m, 3H), 3.27-3.29 (m, 2H), 3.80-3.83 (m, 1H), 4.15-4.31 (m, 1H), 4.31-4.35 (m, 3H), 6.35 (s, 1H), 6.54 (s, 2H), 7.26-7.28 (d, 1H), 7.48 (s, 1H), 7.51-7.54 (m, 2H), 7.78-7.81 (m, 1H), 7.90 (s, 1H), 8.19-8.26 (m, 2H).

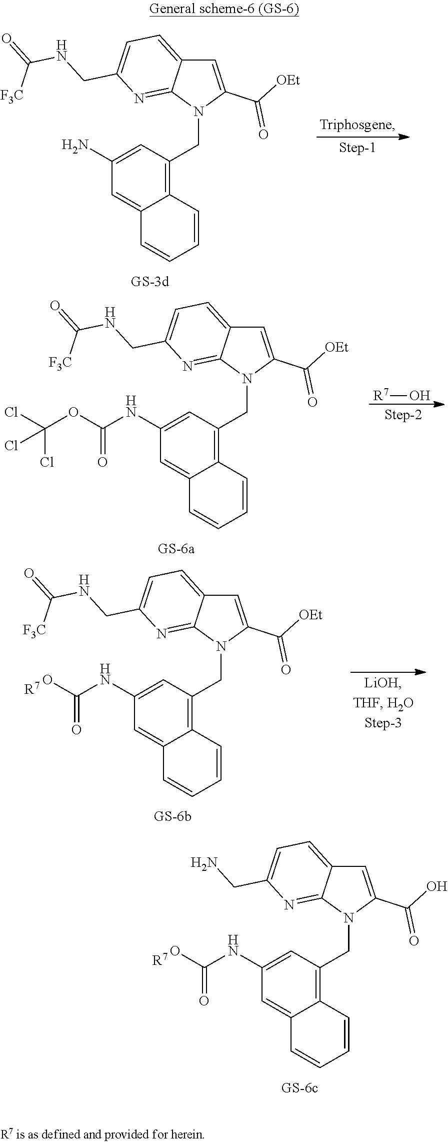

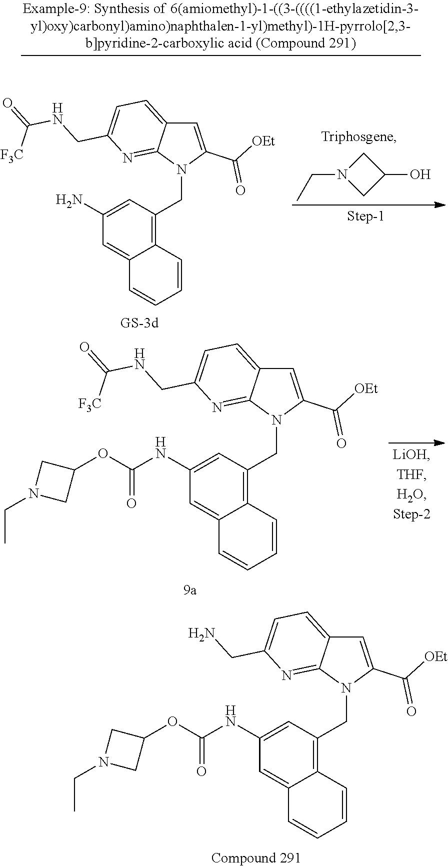

### Step 1: ethyl 1-((3-((((1-ethylazetidin-3-yl)oxy)carbonyl)amino)naphthalen-1-yl)methyl)-6-((2,2,2-trifluoroacetamido)methyl)-1H-pyrrolo[2,3-b]pyridine-2-carboxylate

A solution of triphosgene (0.0309 g, 0.104 mmol) in DCM (4 mL) was cooled to −10° C. and to the solution was added a solution of ethyl 1-((3-aminonaphthalen-1-yl)methyl)-6-((2,2,2-trifluoroacetamido)methyl)-1H-pyrrolo[2,3-b]pyridine-2-carboxylate (0.0611 g, 0.13 mmol) in DCM (4 mL), then Et3N (0.113 mL, 0.78 mmol) was added and stirred at −10° C. for 0.5 h. The mixture was stirred under-10° C. for 0.5 hr. This mixture was added solution of 1-ethylazetidin-3-ol (0.197 g, 0.195 mmol) at −10° C. Then the mixture was stirred at 100° C. for 2 h. The reaction mixture was concentrated and the residue was used for next step without further purification.

Step 2:6-(aminomethyl)-1-((3-((((1-ethylazetidin-3-yl)oxy)carbonyl)amino)naphthalen-1-yl)methyl)-1H-pyrrolo[2,3-b]pyridine-2-carboxylic acid. (Compound 291)

A solution of ethyl 1-((3-((((1-ethylazetidin-3-yl)oxy)carbonyl)amino)naphthalen-1-yl)methyl)-6-((2,2,2-trifluoroacetamido)methyl)-1H-pyrrolo[2,3-b]pyridine-2-carboxylate (0.077 g, 0.13 mmol) in THF (3 mL) and H₂O (1 mL) was added LiOH (0.025 g, 1.04 mmol). The mixture was stirred at 30° C. for 16 h and concentrated. The residue was purified by prep-HPLC to afford the pure title compound (Compound 291) (0.0035 g). LC-MS: 474.2 [M+H]⁺.

The following compounds listed in Table 3 were prepared according to GS-5 or GS-6 by following the similar procedure as described above for Example-5 or Example-6 or Example-7 or Example-8 or Example-9 using appropriate reagents with suitable modifications known to the one skilled in the art.

TABLE 3

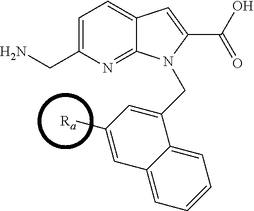

| Compound | Rₐ | Method | Spectral data |
|---|---|---|---|
| 292 | 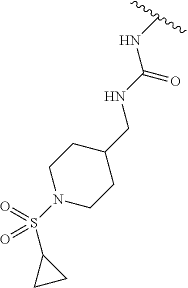 [SMILES: *NC(=O)NCC1CCN(S(=O)(=O)C2CC2)CC1] | GS-5 | LC-MS: 591.4 [M + H]⁺; ¹HNMR(CD₃OD, 400 MHz): δ 0.98-1.01 (bs, 4H), 1.19-1.25 (bs, 2H), 1.56 (bs, 1H), 1.7-1.74 (d, 2H), 2.42 (bs, 1H), 2.77-2.83 (t, 2H), 3.03-3.04 (d, 2H), 3.69-3.72 (d, 2H), 4.33 (s, 2H), 6.139 (s, 1H), 6.52 (s, 2H), 7.26-7.28 (d, 1H), 7.48- 7.51 (m, 3H), 7.74-7.76 (d, 1H), 7.82 (s, 1H), 8.16- 8.27 (d, 2H). |
| 293 | 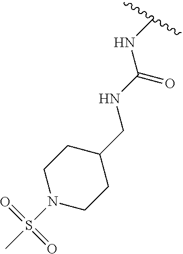 [SMILES: *NC(=O)NCC1CCN(S(C)(=O)=O)CC1] | GS-5 | LC-MS: 565.4 [M + H]⁺; ¹HNMR(CD₃OD, 400 MHz): δ 1.18-1.28 (m, 2H), 1.40-1.75 (m, 3H), 2.62-2.70 (m, 2H), 2.78 (s, 3H), 3.02-3.04 (d, 2H), 3.66-3.70 (d, 2H), 4.33 (s, 2H), 6.12 (s, 1H), 6.52 (s, 2H), 7.28-7.26 (d, 1H), 7.46-7.50 (m, 3H), 8.15-8.18 (m, 2H), 8.25- 8.28 (m, 2H). |
| 294 | 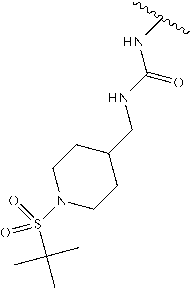 [SMILES: *NC(=O)NCC1CCN(S(=O)(=O)C(C)(C)C)CC1] | GS-5 | LC-MS: 607.40 [M + H]⁺; ¹HNMR(CD₃OD, 400 MHz): δ 1.38 (m, 9H), 1.97-1.98 (d, 5 H), 2.04-2.10 (m, 2H), 2.56 (m, 1H), 2.62 (s, 1H), 2.74-2.75 (d, 3H), 3.75 (s, 1H), 3.81-3.86 (m, 2H), 4.24-4.29 (t, 1H), 7.02 (S, 1H), 7.13-7.18 (m, 1H), 7.39-7.43 (d, 2H), 7.51-7.57 (m, 3H), 8.3 (s, 1H). |
| 295 | 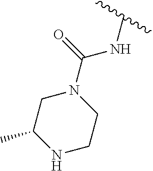 [SMILES: *NC(=O)N1CCN[C@H](C)C1] | GS-6 | LC-MS: 473 [M + H]⁺. |
| 296 | 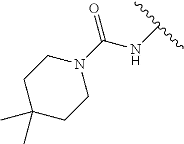 [SMILES: *NC(=O)N1CCC(C)(C)CC1] | GS-6 | LC-MS: 486 [M + H]⁺. |
| 297 | 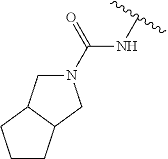 [SMILES: *NC(=O)N1CC2CCCC2C1] | GS-6 | LC-MS: 484 [M+H]⁺. |
| 298 | 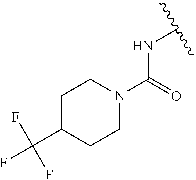 [SMILES: *NC(=O)N1CCC(C(F)(F)F)CC1] | GS-5 | LC-MS: 526.1 [M + H]⁺; ¹HNMR(CD₃OD, 400 MHz): δ 1.35-1.42 (m, 2H), 1.81-1.84 (d, 2H), 2.39-2.42 (m, 1H), 2.72-2.82 (t, 2H), 4.09-4.13 (d, 2H), 4.32 (s, 2H), 6.41 (s, 1H), 6.50 (s, 2H), 7.24-7.26 (d, 1H), 7.47- 7.49 (t, 3H), 7.67 (s, 1H), 7.75-7.77 (m, 1H), 8.15- 8.82 (m, 1H), 8.22-8.24 (d, 1H). |
| 299 | 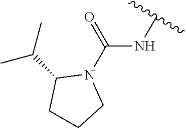 [SMILES: *NC(=O)N1CCC[C@@H]1C(C)C] | GS-6 | LC-MS: 486 [M + H]⁺. |
| 300 | 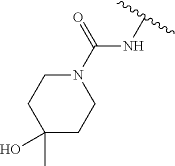 [SMILES: *NC(=O)N1CCC(C)(O)CC1] | GS-6 | LC-MS: 488 [M + H]⁺. |
| 301 | 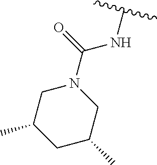 [SMILES: *NC(=O)N1C[C@H](C)C[C@H](C)C1] | GS-6 | LC-MS: 486 [M + H]⁺. |
| 302 | 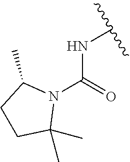 [SMILES: *NC(=O)N1[C@@H](C)CCC1(C)C] | GS-6 | LC-MS: 486 [M + H]⁺. |
| 303 | 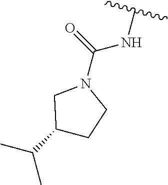 [SMILES: *NC(=O)N1CC[C@H](C(C)C)C1] | GS-6 | LC-MS: 486 [M + H]⁺. |
| 304 | 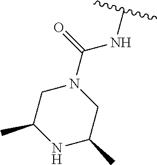 [SMILES: *NC(=O)N1C[C@@H](C)N[C@@H](C)C1] | GS-6 | LC-MS: 487 [M + H]⁺. |
| 305 | 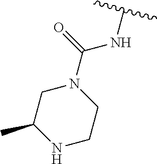 [SMILES: *NC(=O)N1CCN[C@@H](C)C1] | GS-6 | LC-MS: 473 [M + H]⁺. |
| 306 | 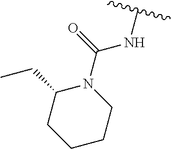 [SMILES: *NC(=O)N1CCCC[C@@H]1CC] | GS-6 | LC-MS: 486 [M + H]⁺. |
| 307 | 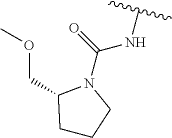 [SMILES: *NC(=O)N1CCC[C@@H]1COC] | GS-6 | LC-MS: 488 [M + H]⁺. |
| 308 | 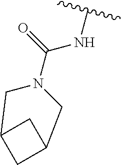 [SMILES: *NC(=O)N1CC2CC(C2)C1] | GS-6 | LC-MS: 470 [M + H]⁺. |
| 309 | 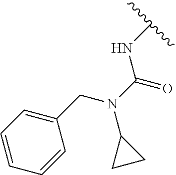 [SMILES: *NC(=O)N(Cc1ccccc1)C1CC1] | GS-6 | LC-MS: 520 [M + H]⁺. |
| 310 | 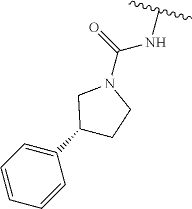 [SMILES: *NC(=O)N1CC[C@H](c2ccccc2)C1] | GS-6 | LC-MS: 520 [M + H]⁺. |
| 311 | 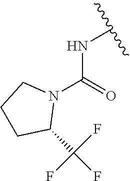 [SMILES: *NC(=O)N1CCC[C@H]1C(F)(F)F] | GS-6 | LC-MS: 512 [M + H]⁺. |
| 312 | 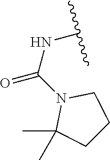 [SMILES: *NC(=O)N1CCCC1(C)C] | GS-6 | LC-MS: 472 [M + H]⁺. |
| 313 | 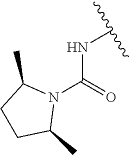 [SMILES: *NC(=O)N1[C@H](C)CC[C@@H]1C] | GS-6 | LC-MS: 472.2 [M + H]⁺. |
| 314 | 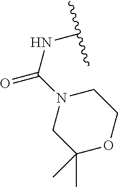 [SMILES: *NC(=O)N1CCOC(C)(C)C1] | GS-6 | LC-MS: 488 M + H]⁺. |
| 315 | 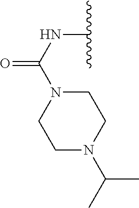 [SMILES: *NC(=O)N1CCN(C(C)C)CC1] | GS-6 | LC-MS: 501 [M + H]⁺. |
| 316 | 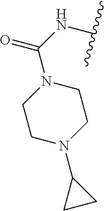 [SMILES: *NC(=O)N1CCN(C2CC2)CC1] | GS-6 | LC-MS: 499 [M + H]⁺. |
| 317 | 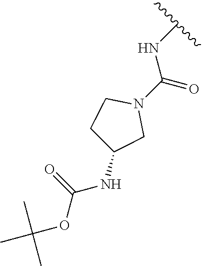 [SMILES: *NC(=O)N1CC[C@@H](NC(=O)OC(C)(C)C)C1] | GS-6 | LC-MS: 559 [M + H]⁺. |
| 318 | 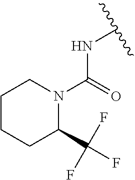 [SMILES: *NC(=O)N1CCCC[C@@H]1C(F)(F)F] | GS-6 | LC-MS: 526 [M + H]⁺. |
| 319 | 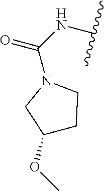 [SMILES: *NC(=O)N1CC[C@H](OC)C1] | GS-6 | LC-MS: 474 [M + H]⁺. |
| 320 | 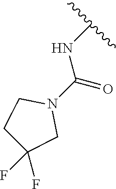 [SMILES: *NC(=O)N1CCC(F)(F)C1] | GS-6 | LC-MS: 480 [M + H]⁺. |
| 321 | 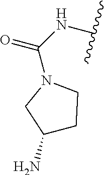 [SMILES: *NC(=O)N1CC[C@H](N)C1] | GS-6 | LC-MS: 459 [M + H]⁺. |
| 322 | 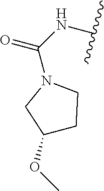 [SMILES: *NC(=O)N1CC[C@H](OC)C1] | GS-6 | LC-MS: 474 [M + H]⁺. |
| 323 | 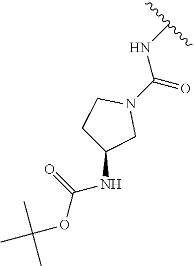 [SMILES: *NC(=O)N1CC[C@H](NC(=O)OC(C)(C)C)C1] | GS-6 | LC-MS: 559 [M + H]⁺. |
| 324 | 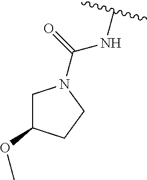 [SMILES: *NC(=O)N1CC[C@@H](OC)C1] | GS-6 | LC-MS: 474 [M + H]⁺. |
| 325 | 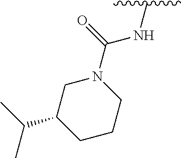 [SMILES: *NC(=O)N1CCC[C@H](C(C)C)C1] | GS-6 | LC-MS: 500 [M + H]⁺. |
| 326 | 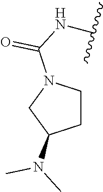 [SMILES: *NC(=O)N1CC[C@@H](N(C)C)C1] | GS-6 | LC-MS: 487 [M + H]⁺. |
| 327 | 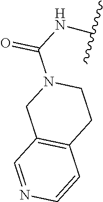 [SMILES: *NC(=O)N1CCc2ccncc2C1] | GS-6 | LC-MS: 507 [M + H]⁺. |
| 328 | 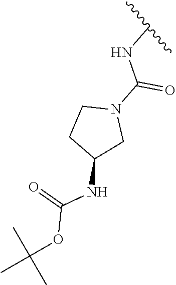 [SMILES: *NC(=O)N1CC[C@H](NC(=O)OC(C)(C)C)C1] | GS-6 | LC-MS: 559 [M + H]⁺. |
| 329 | 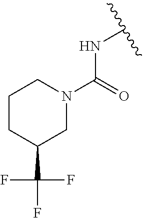 [SMILES: *NC(=O)N1CCC[C@H](C(F)(F)F)C1] | GS-6 | LC-MS: 526 [M + H]⁺. |
| 330 | 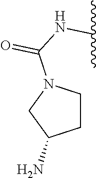 [SMILES: *NC(=O)N1CC[C@H](N)C1] | GS-6 | LC-MS: 459 [M + H]⁺. |
| 331 | 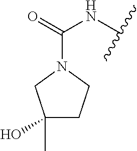 [SMILES: *NC(=O)N1CC[C@](C)(O)C1] | GS-6 | LC-MS: 474 [M + H]⁺. |
| 332 | 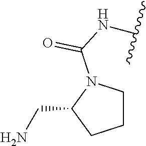 [SMILES: *NC(=O)N1CCC[C@@H]1CN] | GS-6 | LC-MS: 473 [M + H]⁺. |
| 333 | 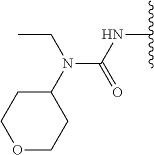 [SMILES: *NC(=O)N(CC)C1CCOCC1] | GS-6 | LC-MS: 502 [M + H]⁺. |
| 334 | 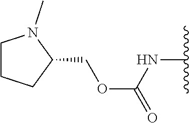 [SMILES: *NC(=O)OC[C@@H]1CCCN1C] | GS-5 | LC-MS: 488.3 [M + H]⁺; ¹HNMR(CD₃OD, 300 MHz): δ 1.86-1.92 (m, 1H), 1.97-2.04 (m, 1H), 2.09-2.15 (m, 1H), 2.26-2.30 (m, 1H), 2.97 (s, 3H), 3.13-3.20 (m, 1H), 3.65-3.67 (m, 2H), 4.14-4.20 (m, 1H), 4.32 (s, 2H), 4.46-4.51 (m, 1H), 6.33 (s, 1H), 6.54 (s, 2H), 7.26-7.29 (d, 1H), 7.50-7.54 (m, 3H), 7.78-7.82 (m, 1H), 7,94 (s, 1H), 8.19-8.27 (m, 2H). |
| 335 | 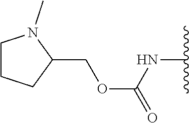 [SMILES: *NC(=O)OCC1CCCN1C] | GS-5 | LC-MS: 488.2 [M + H]⁺; ¹HNMR(CD₃OD, 400 MHz): δ 1.88-1.91 (m, 1H), 1.99-2.05 (m, 1H), 2.12-2.17 (m, 1H), 2.29-2.33 (m, 1H), 2.99 (s, 3H), 3.14-3.21 (m, 1H), 3.63-3.69 (m, 2H), 4.15-4.20 (m, 1H), 4.34 (s, 2H), 4.48-4.52 (dd, 1H), 6.35 (s, 1H), 6.56 (s, 2H), 7.28-7.30 (d, 1H), 7.52-7.56 (m, 3H), 7.81-7.83 (m, 1H), 7.95 (s, 1H), 8.21-8.23 (m, 1H), 8.26-8.28 (d, 1H). |
| 336 | 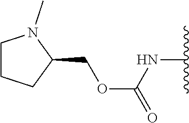 [SMILES: *NC(=O)OC[C@H]1CCCN1C] | GS-5 | LC-MS: 488.2 [M + H]⁺; ¹HNMR (CD₃OD, 400 MHz): δ 1.80-2.40 (m, 4H), 2.98 (s, 3H), 3.15-3.17 (q, 1H), 3.64-3.68 (m, 2H), 4.15-4.20 (q, 1H), 4.33 (s, 2H), 4.46-4.50 (m, 1H), 6.34 (s, 1H), 6.54 (s, 2H), 7.26- 7.28 (d, 1H), 7.51-7.54 (m, 3H), 7.79-7.81 (m, 1H), 7.94 (s, 1H), 8.19-8.22 (m, 1H), 8.25-8.27 (d, 1H). |
| 337 | 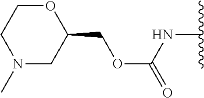 [SMILES: *NC(=O)OC[C@H]1CN(C)CCO1] | GS-5 | LC-MS: 504.4 [M + H]⁺; ¹HNMR(CD₃OD, 300 MHz): δ 2.89-3.09 (m, 5H), 3.38-3.49 (m, 2H), 3.72-3.81 (m, 1H), 3.89-3.92 (m, 1H), 4.05-4.15 (m, 3H), 4.33 (s, 2H), 6.34 (s, 1H), 6.53 (s, 2H), 7.26-7.28 (d, 1H), 7.50-7.53 (m, 3H), 7.77-7.80 (m, 1H), 7.87 (s, 1H), 8.18-8.27 (m, 2H). |
| 338 | 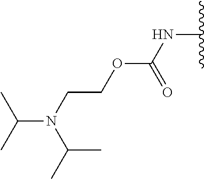 [SMILES: *NC(=O)OCCN(C(C)C)C(C)C] | GS-6 | LC-MS: 518 [M + H]⁺. |
| 339 | 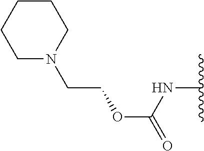 | GS-6 | LC-MS: 516 [M + H]⁺. |
| 340 | 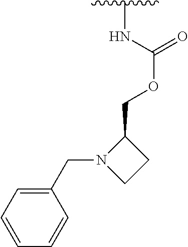 [SMILES: *NC(=O)OC[C@H]1CCN1Cc1ccccc1] | GS-6 | LC-MS: 550 [M + H]⁺. |
| 341 | 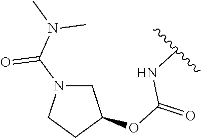 [SMILES: *NC(=O)O[C@H]1CCN(C(=O)N(C)C)C1] | GS-6 | LC-MS: 531 [M + H]⁺. |
| 342 | 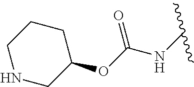 [SMILES: *NC(=O)O[C@@H]1CCCNC1] | GS-6 | LC-MS: 474 [M + H]⁺. |
| 343 | 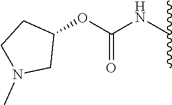 [SMILES: *NC(=O)O[C@H]1CCN(C)C1] | GS-6 | LC-MS: 474 [M + H]⁺. |

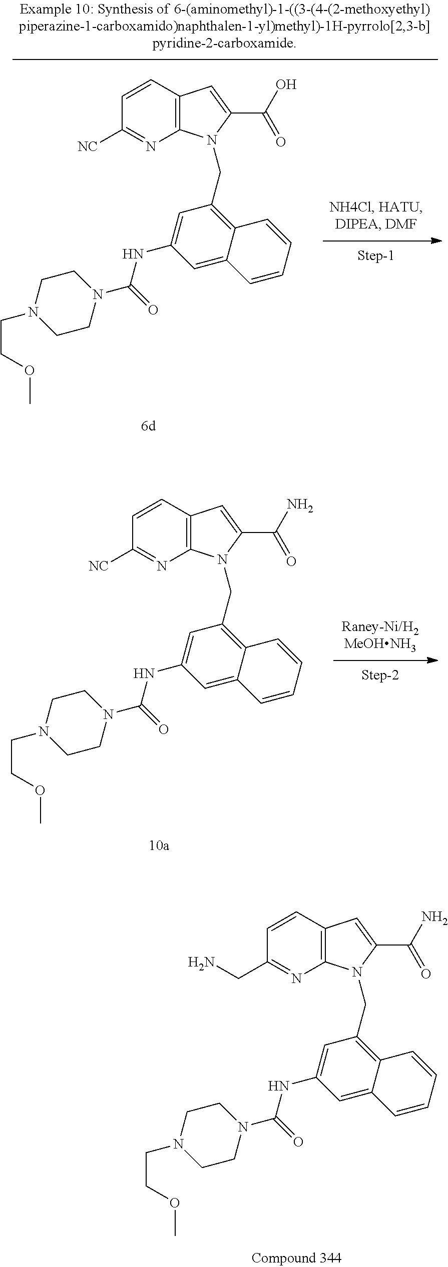 [SMILES: COCCN1CCN(C(=O)Nc2cc(Cn3c(C(=O)O)cc4ccc(C#N)nc43)c3ccccc3c2)CC1 ; COCCN1CCN(C(=O)Nc2cc(Cn3c(C(N)=O)cc4ccc(C#N)nc43)c3ccccc3c2)CC1 ; COCCN1CCN(C(=O)Nc2cc(Cn3c(C(N)=O)cc4ccc(CN)nc43)c3ccccc3c2)CC1]

### Step-1: Synthesis of 6-cyano-1-(3-(4-(2-methoxyethyl)piperazine-1-carboxamido)naphthalen-1-yl)methyl)-1H-pyrrolo[2,3-b]pyridine-2-carboxamide

The experimental procedure was the same as described in Step-9 of Example-1 with appropriate variations in coupling methods, reactants, quantities of reagents, and solvents to obtain the compound 10a (0.32 g, crude). LC-MS: 516.30 [M+H]⁺.

### Step-2: Synthesis of 6-(aminomethyl)-1-((3-(4-(2-methoxyethyl)piperazine-1-carboxamido)naphthalen-1-yl)methyl)-1H-pyrrolo[2,3-b]pyridine-2-carboxamide. (Compound 344)

The experimental procedure was the same as described in Step-10 of Example-1 with appropriate variations in coupling methods, reactants, quantities of reagents, and solvents to obtain the title compound (Compound 344) (0.1 g, 29%). LC-MS: 516.3 [M+H]⁺; ¹HNMR (CD₃OD, 300 MHz): δ 3.29-3.40 (bs, 10H), 3.43-3.45 (s, 3H), 3.69-3.73 (t, 2H), 4.33 (s, 2H), 6.504 (s, 2H), 6.605 (s, 1H), 7.23-7.29 (m, 2H), 7.47-7.50 (m, 2H), 7.69 (s, 1H), 7.757 (m, 1H), 8.17-8.20 (m, 2H).

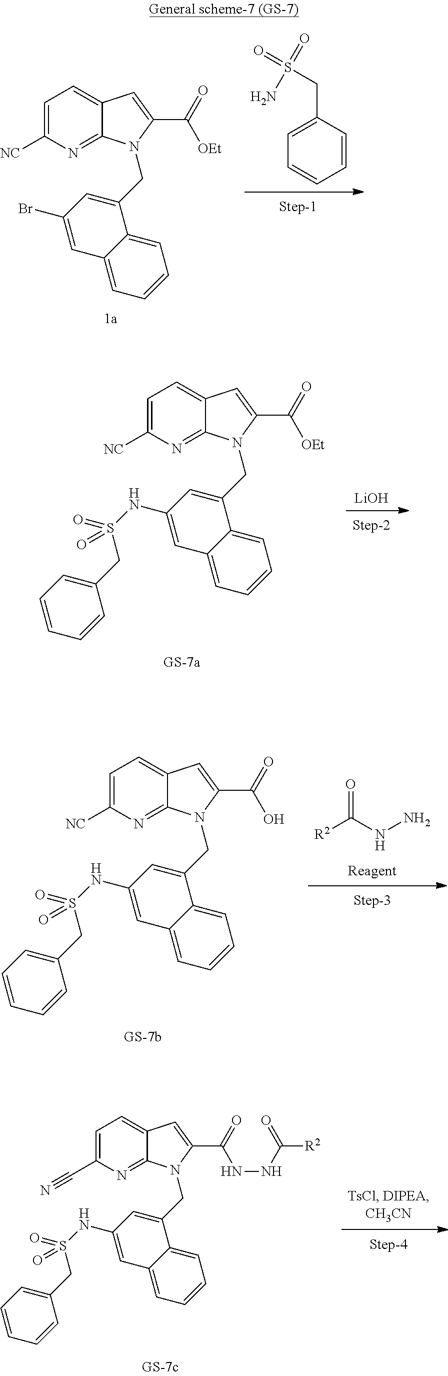

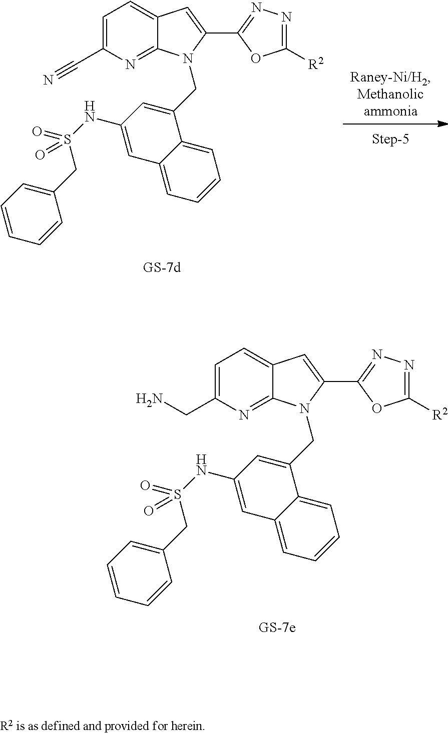

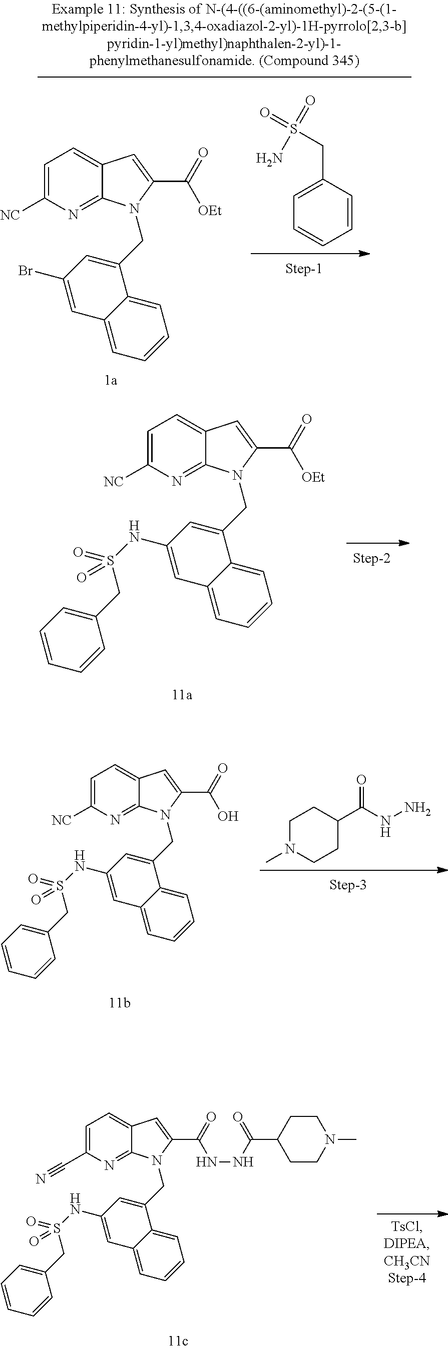

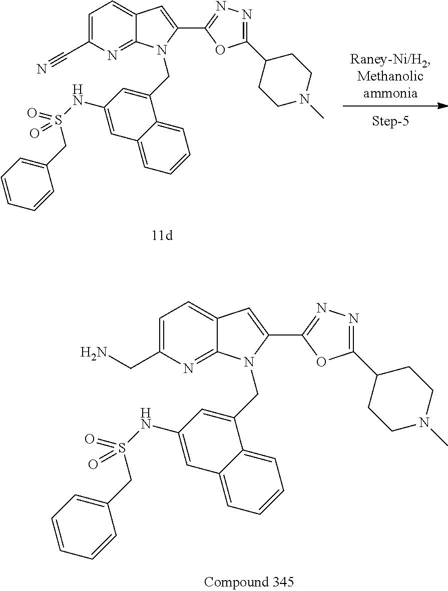 [SMILES: CN1CCC(c2nnc(-c3cc4ccc(C#N)nc4n3Cc3cc(NS(=O)(=O)Cc4ccccc4)cc4ccccc34)o2)CC1 ; "Raney-Ni/H2,Methanolicammonia" ; CN1CCC(c2nnc(-c3cc4ccc(CN)nc4n3Cc3cc(NS(=O)(=O)Cc4ccccc4)cc4ccccc34)o2)CC1]

### Step-1: Synthesis of ethyl 6-cyano-1-((3-((phenylmethyl)sulfonamido)naphthalen-1-yl)methyl)-1H-pyrrolo[2,3-b]pyridine-2-carboxylate

The experimental procedure was the same as described in Step-3 of Example-2 with appropriate variations in coupling methods, reactants, quantities of reagents, and solvents to obtain the compound 11a. LC-MS: 436.10 [M+2]⁺.

### Step-2: Synthesis of 6-cyano-1-((3-((phenylmethyl)sulfonamido)naphthalen-1-yl)methyl)-1H-pyrrolo[2,3-b]pyridine-2-carboxylic acid

The experimental procedure was the same as described in Step-4 of Example-2 with appropriate variations in coupling methods, reactants, quantities of reagents, and solvents to obtain the compound 11b. LC-MS: 405.70 [M−H]⁻.

### Step-3: Synthesis of N-(4-((6-cyano-2-(2-(1-methylpiperidine-4-carbonyl) hydrazine-1-carbonyl)-1H-pyrrolo[2,3-b]pyridin-1-yl)methyl)naphthalen-2-yl)-1-phenylmethanesulfonamide

To a stirred suspension of 6-cyano-1-((3-((phenylmethyl)sulfonamido)naphthalen-1-yl)methyl)-1H-pyrrolo[2,3-b]pyridine-2-carboxylic acid (0.5 g, 1.0 mmol) and 1-methylpiperidine-4-carbohydrazide (0.19 g, 1.2 mmol) and K₂CO₃ (0.41 g, 3.02 mmol) in DMF (5 mL) was added HATU (0.35 g, 1.51 mmol)). The mixture was stirred for 5 minutes at RT and heated to 50° C. for 2 h. Then the reaction mixture was cooled to RT and to the mixture was added water to form solids that were filtered, washed with water and dried to get the crude mass. The crude compound was purified in combi flash column chromatograph using 0-5% methanol in DCM as eluent. This afforded the pure title compound 11c (0.45 g, 70.2%). LC-MS: 636.30[M+H]⁺.

### Step-4: Synthesis of N-(4-((6-cyano-2-(5-(1-methylpiperidin-4-yl)-1,3,4-oxadiazol-2-yl)-1H-pyrrolo[2,3-b]pyridin-1-ylmethyl)naphthalen-2-yl)-1-phenylmethanesulfonamide

To a stirred solution of N-(4-((6-cyano-2-(2-(1-methylpiperidine-4-carbonyl) hydrazine-1-carbonyl)-1H-pyrrolo[2,3-b]pyridin-1-yl)methyl)naphthalen-2-yl)-1-phenylmethanesulfonamide (0.05 g, 0.07 mmol) in acetonitrile (5 mL) was added DIPEA (0.15 g, 1.49 mmol)) under argon atmosphere. P-toluene sulfonyl chloride (0.015 g, 0.99 mmol) was added and the reaction mixture was stirred at RT for an hour. The reaction mixture was evaporated under reduced pressure, the residue obtained was washed with di ethyl ether and dried to yield the title compound 11d (0.04 g, 81.97%). LC-MS: 618.40 [M+H]⁺.

### Step-5: Synthesis of N-(4-((6-(aminomethyl)-2-(5-(1-methylpiperidin-4-yl)-1,3,4-oxadiazol-2-yl)-1H-pyrrolo[2,3-b]pyridin-1-yl)methyl)naphthalen-2-yl)-1-phenylmethanesulfonamide. (Compound 345)

To a solution of N-(4-((6-cyano-2-(5-(1-methylpiperidin-4-yl)-1,3,4-oxadiazol-2-yl)-1H-pyrrolo[2,3-b]pyridin-1-yl)methyl)naphthalen-2-yl)-1-phenylmethanesulfonamide (0.25 g, 0.45 mmol) in methnolic ammonia (10 mL) was added Raney nickel (0.119 g, 2.02 mmol) and stirred under positive pressure of hydrogen using a bladder. After 6 h, the reaction mixture was filtered through a Celite pad, and the filtrate was concentrated to get residue. The residue was purified in preparative HPLC to get the pure title compound (Compound 345) (0.05 g, 20%). LC-MS: 622.40 [M+H]⁺; ¹HNMR (CD₃OD, 400 MHz): δ 1.88-1.91 (q, 2H), 2.18-2.21 (d, 2H), 2.87 (s, 3H), 3.02-3.22 (m, 4H), 3.52-3.55 (d, 2H), 4.09 (s, 2H), 4.37 (s, 2H), 6.32 (s, 1H), 6.67 (s, 2H), 7.00-7.07 (m, 2H), 7.17-7.26 (m, 3H), 7.34-7.36 (m, 2H), 7.58-7.63 (m, 2H), 7.79-7.81 (d, 1H), 8.27-8.31 (m, 2H).

The following compounds listed in Table 4 were prepared according to GS-7 by following the similar procedure as described above for Example-11 using appropriate reagents with suitable modifications known to the one skilled in the art.

TABLE 4

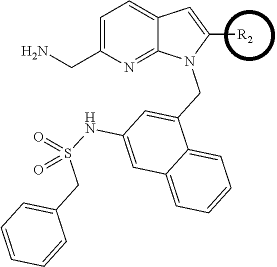

| Compound | R₂ | Reagent used | Spectral data |
|---|---|---|---|
| 346 | 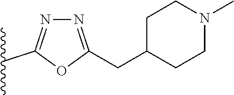 [SMILES: *c1nnc(CC2CCN(C)CC2)o1] | 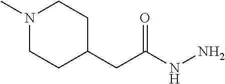 | LC-MS: 636.4 [M + H]⁺; ¹HNMR(CD₃OD, 400 MHz): δ 1.37-1.41 (d, 2H), 1.77-1.81 (d, 2H), 2.68-2.71 (d, 2H), 2.76-2.80 (m, 6H), 3.31-3.34 (m, 2H), 4.09 (s, 2H), 4.35 (s, 2H), 6.30 (s, 1H), 6.65 (s, 2H), 7.01-7.06(d, 2H), 7.06-7.08 (d, 1H), 7.17-7.25 (m, 3H), 7.32- |
| | | | 7.36 (t, 2H), 7.55-7.60 (m, 2H), 7.79-7.81 (d, |
| | | | 1H), 8.26-8.31 (m, 2H). |
| 347 | 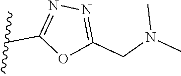 [SMILES: *c1nnc(CN(C)C)o1] | 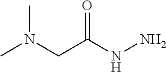 | LC-MS: 582.4 [M + H]⁺; ¹HNMR(CD₃OD, 400 MHz): δ 2.81 (s, 6H), 4.00 (s, 2H), 4.27 (s, 2H), 4.60 (s, 2H), 6.24 (s, 1H), 6.60 (s, 2H), 6.93-6.94 (d, 2H), 7.07-7.15 (m, 3H), 7.25-7.27 (d, 2H), 7.48-7.54 (m, 3H), 7.69- |
| | | | 7.71 (dd, 1H), 8.18-8.25 (m, 2H). |
| 348 | 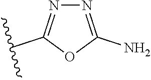 [SMILES: *c1nnc(N)o1] | 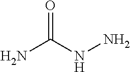 | LC-MS: 540.3 [M + H]⁺; ¹HNMR(CD₃OD, 400 MHz): 8 4.13 (s, 2H), 4.31 (s, 2H), 6.28 (s, 1H), 6.61 (s, 2H), 6.99-7.00 (d, 2H), 7.15- 7.30 (m, 4H), 7.42 (s, 1H), 7.55-7.57 (m, 2H), 7.77-7.79 (dd, 1H), 8.23-8.25 (d, 2H). |

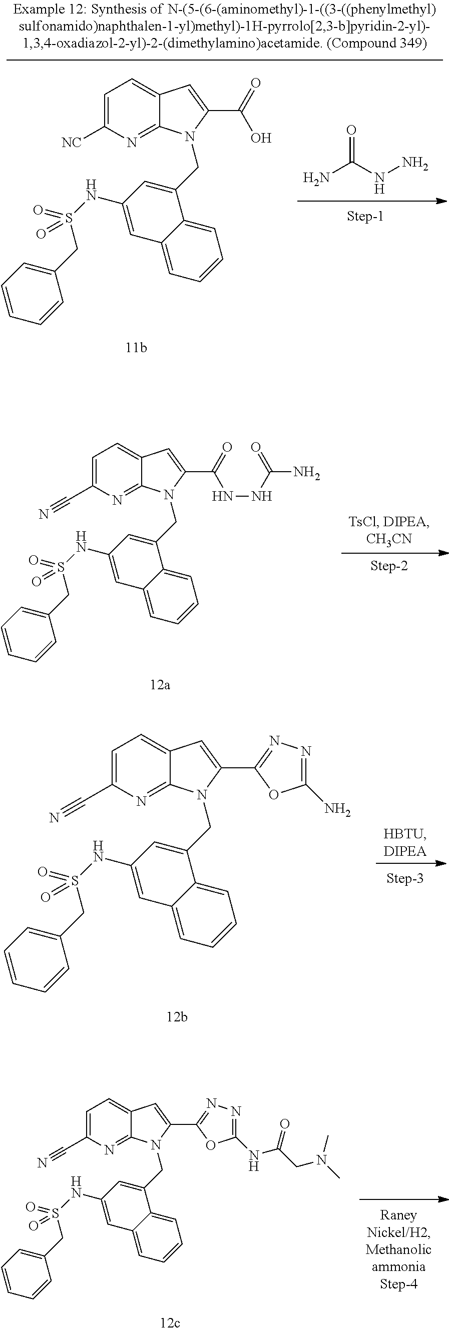

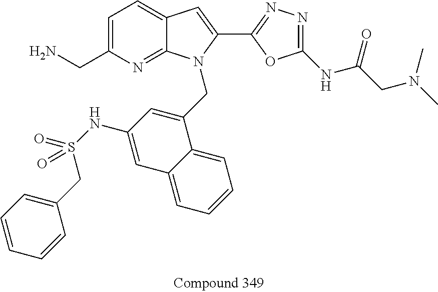 [SMILES: CN(C)CC(=O)Nc1nnc(-c2cc3ccc(CN)nc3n2Cc2cc(NS(=O)(=O)Cc3ccccc3)cc3ccccc23)o1]

### Step-1: Synthesis of 2-(6-cyano-1-(3-((phenylmethyl)sulfonamido)naphthalen-1-yl)methyl)-1H-pyrrolo[2,3-b]pyridine-2-carbonyl) hydrazine-1-carboxamide

The experimental procedure was the same as described in Step-3 of Example-5 with appropriate variations in coupling methods, reactants, quantities of reagents, and solvents to obtain the compound 12a. LC-MS: 554.10 [M+H]⁺.

### Step-2: Synthesis of N-(4-((2-(5-amino-1,3,4-oxadiazol-2-yl)-6-cyano-1H-pyrrolo[2,3-b]pyridin-1-yl)methyl)naphthalen-2-yl)-1-phenylmethanesulfonamide

The experimental procedure was the same as described in Step-4 of Example-5 with appropriate variations in coupling methods, reactants, quantities of reagents, and solvents to obtain the compound 12b. LC-MS: 534.90 [M−H]⁻.

### Step-3: Synthesis of N-(5-(6-cyano-1-((3-((phenylmethyl)sulfonamido)naphthalen-1-yl)methyl)-1H-pyrrolo[2,3-b]pyridin-2-yl)-1,3,4-oxadiazol-2-yl)-2-(dimethylamino)acetamide

The experimental procedure was the same as described in Step-9 of Example-1 with appropriate variations in coupling methods, reactants, quantities of reagents, and solvents to obtain the compound 11c. LC-MS: 621.1 [M+H]⁺.

### Step-4: Synthesis of N-(5-(6-(aminomethyl)-1-(3-((phenylmethyl)sulfonamido)naphthalen-1-yl)methyl)-1H-pyrrolo[2,3-b]pyridin-2-yl)-1,3,4-oxadiazol-2-yl)-2-(dimethylamino)acetamide. (Compound 349)

The experimental procedure was the same as described in Step-5 of Example-5 with appropriate variations in coupling methods, reactants, quantities of reagents, and solvents to obtain the compound (Compound 349) (0.1 g, 100%). LC-MS: 625.40 [M+H]⁺; ¹HNMR (CD₃OD, 400 MHz): δ 2.89 (s, 6H), 4.08 (s, 2H), 4.20 (s, 2H), 4.34 (s, 2H), 6.30 (s, 1H), 6.63 (s, 2H), 6.98-6.99 (m, 2H), 7.16-7.23 (m, 3H), 7.31-7.35 (m, 2H), 7.47 (s, 1H), 7.54-7.56 (m, 2H), 7.70-7.80 (m, 1H), 8.20-8.30 (m, 2H).

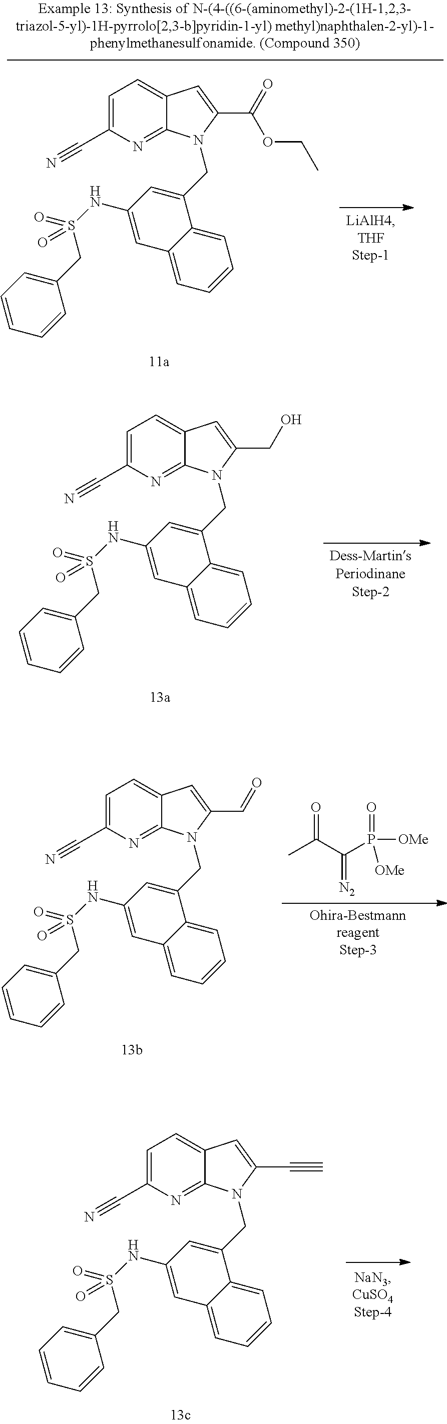

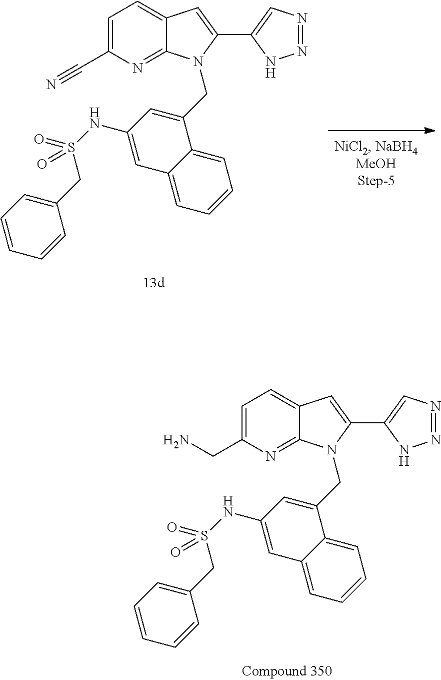 [SMILES: N#Cc1ccc2cc(-c3cnn[nH]3)n(Cc3cc(NS(=O)(=O)Cc4ccccc4)cc4ccccc34)c2n1 ; NCc1ccc2cc(-c3cnn[nH]3)n(Cc3cc(NS(=O)(=O)Cc4ccccc4)cc4ccccc34)c2n1]

### Step-1: Synthesis of N-(4-((6-cyano-2-(hydroxymethyl)-1H-pyrrolo[2,3-b]pyridin-1-yl)methyl)naphthalen-2-yl)-1-phenylmethanesulfonamide

To a stirred solution of ethyl 6-cyano-1-((3-((phenylmethyl)sulfonamido)naphthalen-1-yl)methyl)-1H-pyrrolo[2,3-b]pyridine-2-carboxylate (1.5 g, 2.86 mmol) in THF (20 mL) was added at 0° C. LiAlH₄ (1.3 mL, 2.3 M, 2.86 mmol) and the reaction was stirred for 2 h. After the completion of the reaction, Sat. Potassium sodium L-tartrate (2 mL) was added at 0° C. and the mixture was extracted with EtOAc. The extracts were washed with saturated aqueous NaCl, dried over Na₂SO₄, and concentrated under reduced pressure. The residue was purified by combi flash column chromatography, eluting with 40% ethyl acetate in hexane to yield the title compound 13a as a yellow solid (1.0 g, 91.9%). LC-MS: 483.05 [M+H]⁺.

### Step-2: Synthesis of N-(4-(6-cyano-2-formyl-1H-pyrrolo[2,3-6]pyridin-1-yl)methyl)naphthalen-2-yl)-1-phenylmethanesulfonamide

To a stirred solution of N-(4-((6-cyano-2-(hydroxymethyl)-1H-pyrrolo[2,3-b]pyridin-1-yl)methyl)naphthalen-2-yl)-1-phenylmethanesulfonamide (1.0 g, 2.07 mmol) in DCM (10 mL) and THF (10 mL) was added Des-Martin's Periodinane (2.64 g, 6.22 mmol) at 0° C. and stirred for 3 h. After the completion of the reaction, Sat. Na2S2O3 (2 mL) was added at 0° C. and the mixture was extracted with EtOAc. The extracts were washed with saturated aqueous NaCl, dried over Na₂SO₄, and concentrated under reduced pressure. The residue was purified by combi flash column chromatography, eluting with 40% ethyl acetate in hexane to yield the title compound as a yellow solid 13b (0.95 g, 95.4%). LC-MS: 481.00 [M+H]⁺.

### Step-3: Synthesis of N-(4-(6-cyano-2-ethynyl-1H-pyrrolo[2,3-b]pyridin-1-yl)methyl)naphthalen-2-yl)-1-phenylmethanesulfonamide

To a stirred solution of N-(4-((6-cyano-2-formyl-1H-pyrrolo[2,3-b]pyridin-1-yl)methyl)naphthalen-2-yl)-1-phenylmethanesulfonamide (0.77 g, 1.46 mmol) in MeOH (5.0 mL) was added at 0° C. Ohira-Bestman's Reagent (0.42 g, 1.50 mmol) in THF (5 mL) and K2CO3 (400 mg, 2.91 mmol), and the reaction was stirred for 3 h. After the completion of the reaction, water (20 mL) was added at 0° C. and the mixture was extracted with EtOAc. The EtOAc extracts were washed with saturated aqueous NaCl dried over Na₂SO₄ and concentrated. The residue was purified by combi flash column chromatography, eluting with 40% ethyl acetate in hexane to yield the title compound 13c as a yellow solid (0.4 g, 51.9%). LC-MS: 477.05 [M+H]⁺.

### Step-4: Synthesis of N-(4-((6-cyano-2-(1H-1,2,3-triazol-5-yl)-1H-pyrrolo[2,3-6]pyridin-1-yl)methyl)naphthalen-2-yl)-1-phenylmethanesulfonamide

To a stirred solution of N-(4-((6-cyano-2-ethynyl-1H-pyrrolo[2,3-b]pyridin-1-yl)methyl)naphthalen-2-yl)-1-phenylmethanesulfonamide (0.3 g, 0.62 mmol) in BuOH:Water (2:1, 5 mL) was added NaN3 (0.05 g, 0.755 mmol) and CuSO4 (0.01 g, 0.063 mmol), and the reaction was stirred for overnight. The mixture was then concentrated under reduced pressure, the residue was taken for next reaction without purification. (0.35 g, crude compound 13d). LC-MS: 520.0 [M+H]⁺.

### Step-5: Synthesis of N-(4-((6-(aminomethyl)-2-(1H-1,2,3-triazol-5-yl)-1H-pyrrolo[2,3-b]pyridin-1-yl)methyl)naphthalen-2-yl)-1-phenylmethanesulfonamide. (Compound 350)

To a stirred suspension of N-(4-((6-cyano-2-(1H-1,2,3-triazol-5-yl)-1H-pyrrolo[2,3-b]pyridin-1-yl)methyl)naphthalen-2-yl)-1-phenylmethanesulfonamide (0.160 g, 0.308 mmol) was added NiCl₂(H₂O)₆ (0.074 g, 0.308 mmol) and NaBH4 (0.081 g, 2.16 mmol). The reaction mixture was kept stirred for 6 h after that evaporated under reduced pressure. The obtained residue was triturated with 5% diethyl ether in pentane, dried under reduced pressure to get the residue which was purified by preparative HPLC (Method: Column-Agilent (150×20 mm), 5 μm; Mobile Phase A: 0.05% TFA in Water, B: Acetonitrile; Flow rate: 18 ml/min; % B—0.1 at 20 minutes, 10 at 65 minutes.) afforded the title compound (Compound 350) (0.01 g, 6.1%) as a TFA Salt. LC-MS: $24.30 [M+H]⁺; ¹HNMR (CD₃OD, 400 MHz): δ 3.98 (s, 2H), 4.20 (s, 2H), 6.24 (s, 1H), 6.45 (s, 2H), 6.82 (d, 2H), 7.00-7.02 (m, 5H), 7.30 (s, 1H), 7.44 (m, 2H), 7.68 (m, 2H), 7.93 (s, 1H), 8.06 (d, 1H), 8.28 (d, 1H).

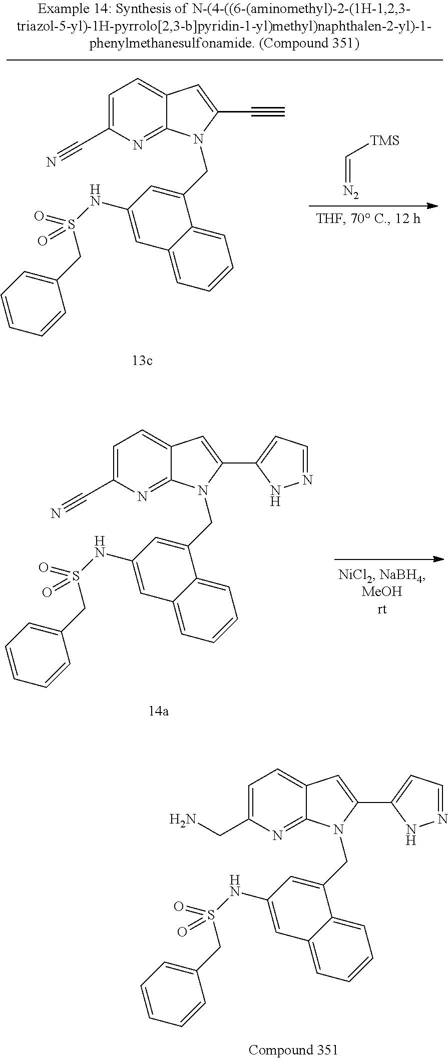

### Step 1: N-(4-((6-cyano-2-(1H-pyrazol-5-yl)-1H-pyrrolo[2,3-b]pyridin-1-yl)methyl) naphthalen-2-yl)-1-phenylmethanesulfonamide

To a stirred solution of N-(4-((6-cyano-2-ethynyl-1H-pyrrolo[2,3-b]pyridin-1-yl)methyl)naphthalen-2-yl)-1-phenylmethanesulfonamide (0.080 g, 0.167 mmol) in THE (1.0 mL) was added Diazomethane (0.1 mL, 0.201 mmol) and the reaction was heated at 90° C. for 12 h. The reaction mixture was concentrated to yield the title compound 14a (0.15 g, crude), this was used for next step. LC-MS: 519.30 [M+H]⁺.

### Step 2: N-(4-(6-(aminomethyl)-2-(1H-pyrazol-5-yl)-1H-pyrrolo[2,3-b]pyridin-1-yl)methyl) naphthalen-2-yl)-1-phenylmethanesulfonamide. (Compound 351)

To a stirred suspension of N-(4-((6-cyano-2-(1H-pyrazol-5-yl)-1H-pyrrolo[2,3-b]pyridin-1-yl)methyl)naphthalen-2-yl)-1-phenylmethanesulfonamide (0.150 g, 0.289 mmol) was added NiCl2(H2O)6 (0.067 g, 0.289 mmol) and NaBH4 (0.077 g, 2.02 mmol). The reaction mixture was kept stirred for 6 h after that evaporated under reduced pressure. The obtained residue was triturated with 5% diethyl ether in pentane, dried under reduced pressure to give the product which was purified by preparative HPLC method given below afforded the title compound (Compound 351) (0.045 g, 30%) as a TFA Salt. (Preparative HPLC Method: Column: LUNA OMEGA (250×21.2 mm) 5μ; Mobile Phase A: 0.05% TFA in Water, B: Acetonitrile; Flow rate: 18 ml/min; % B—20 at 0 minute, 30 at 2 minutes, 50 at 10 minutes). LC-MS: 523.30 [M+H]⁺; ¹HNMR (CD₃OD, 400 MHz): δ 4.07 (s, 2H), 4.27 (s, 2H), 6.38 (s, 1H), 6.50 (s, 2H), 6.90-6.94 (dd, 2H), 7.04-7.080 (m, 3H), 7.17-7.21 (m, 2H), 7.41 (s, 1H), 7.52-7.56 (m, 3H), 7.78 (s, 1H), 8.20-8.24 (d, 1H), 8.27-8.29 (d, 1H).

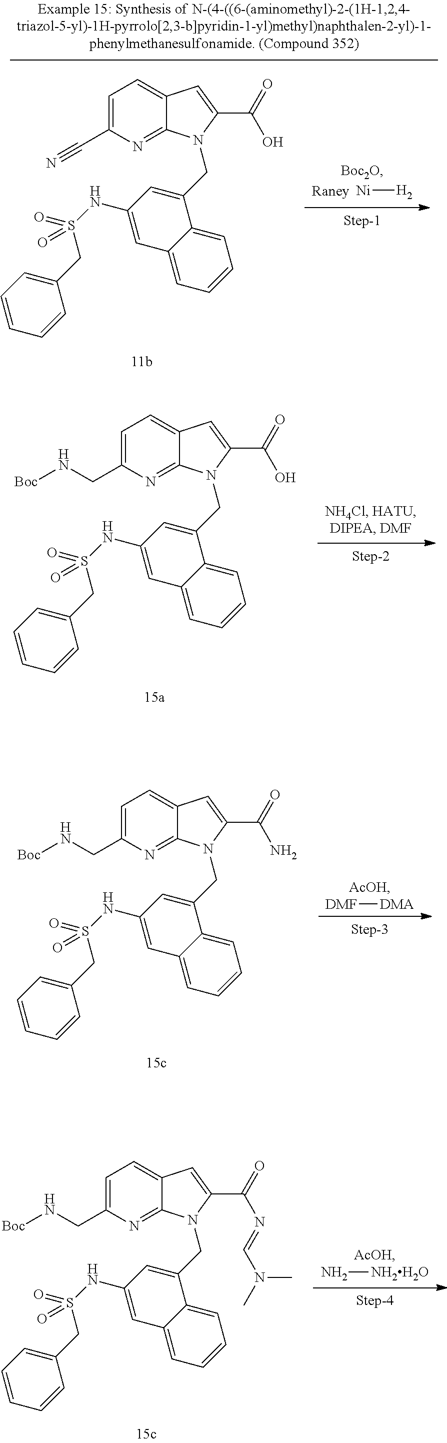

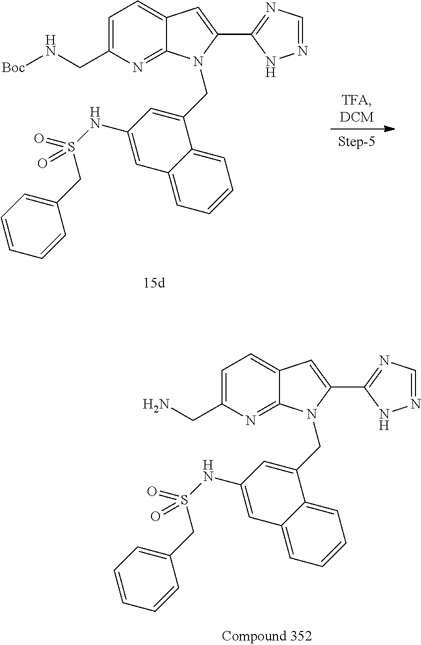

### Step-1: Synthesis of 6-(((tert-butoxycarbonyl)amino)methyl)-1-((3-((phenylmethyl)sulfonamido)naphthalen-1-yl)methyl)-1H-pyrrolo[2,3-b]pyridine-2-carboxylic acid

The experimental procedure was the same as described in Step-1 of Example-2 with appropriate variations in coupling methods, reactants, quantities of reagents, and solvents to obtain the title compound 15a (0.4 g, 34.8%). LC-MS: 601.30 [M+H]⁺.

### Step-2: Synthesis of tert-butyl ((2-carbamoyl-1-((3-((phenylmethyl)sulfonamido)naphthalen-1-yl)methyl)-1H-pyrrolo[2,3-b]pyridin-6-yl)methyl)carbamate

The experimental procedure was the same as described in Step-8 of Example-2 with appropriate variations in coupling methods, reactants, quantities of reagents, and solvents to obtain the title compound 15b (0.38 g, 95.1%). LC-MS: 600.0 [M+H]⁺.

### Step-3: Synthesis of tert-butyl (E)-(2-(((dimethylamino)methylene) carbamoyl)-1-((3-((phenylmethyl)sulfonamido)naphthalen-1-yl)methyl)-1H-pyrrolo[2,3-b]pyridin-6-yl)methyl)carbamate

A mixture of tert-butyl ((2-carbamoyl-1-((3-((phenylmethyl)sulfonamido)naphthalen-1-yl)methyl)-1H-pyrrolo[2,3-b]pyridin-6-yl)methyl)carbamate (0.22, 0.36 mmol) and DMF-DMA (6 mL) was added glacial acetic acid (0.02 mL) and heated to 110° C. for 3 h. The reaction mixture was concentrated and the residue was washed with diethyl ether to yield the title compound 15c (0.23 g, 95.7%). LC-MS: 653.1 [M−H]⁻.

### Step-4: Synthesis of tert-butyl ((1-((3-((phenylmethyl)sulfonamido)naphthalen-1-yl)methyl)-2-(1H-1,2,4-triazol-5-yl)-1H-pyrrolo[2,3-b]pyridin-6-yl)methyl)carbamate

A solution of tert-butyl (E)-((2-(((dimethylamino)methylene) carbamoyl)-1-((3-((phenylmethyl)sulfonamido)naphthalen-1-yl)methyl)-1H-pyrrolo[2,3-b]pyridin-6-yl)methyl)carbamate (0.1 g, 0.153 mmol) in glacial acetic acid (1 mL) was added hydrazine hydrate (0.04 mL) and heated to 110° C. in a microwave reactor for 3 h. The reaction mixture was diluted with ethyl acetate, washed with saturated NaHCO₃ solution followed by washing with brine. The organic portion was dried over Na₂SO₄ and concentrated to yield the title compound 15d (0.065 g, 68.1%). LC-MS: 624.30 [M+H]⁺.

### Step-5: Synthesis of N-(4-(6-(aminomethyl)-2-(1H-1,2,4-triazol-5-yl)-1H-pyrrolo[2,3-b]pyridin-1-yl)methyl)naphthalen-2-yl)-1-phenylmethanesulfonamide. (Compound 352)

The experimental procedure was the same as described in Step-6 of Example-4 with appropriate variations in coupling methods, reactants, quantities of reagents, and solvents to obtain the compound (Compound 352) (0.015 g, 35.8%). LC-MS: 524.3 [M+H]⁺; ¹HNMR (CD₃OD, 400 MHz): δ 4.05 (s, 2H), 4.29 (s, 2H), 6.26 (s, 1H), 6.70 (s, 2H), 6.89-6.91 (d, 2H), 7.07-7.11 (t, 2H), 7.18-7.25 (m, 2H), 7.35 (s, 2H), 7.50-7.56 (m, 2H), 7.73-7.76 (m, 1H), 8.17-8.19 (d, 1H), 8.22-8.24 (d, 1H), 8.43 (bs, 1H).

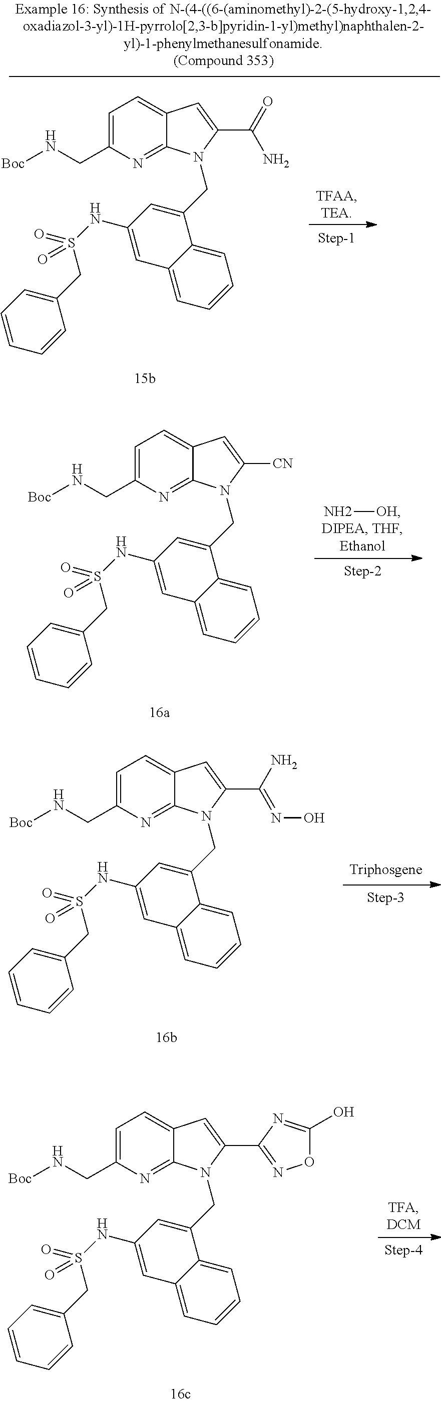

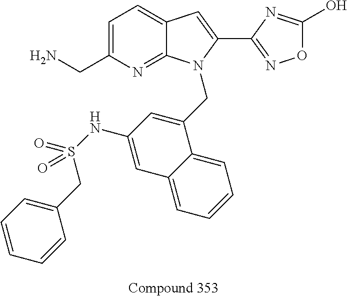 [SMILES: NCc1ccc2cc(-c3noc(O)n3)n(Cc3cc(NS(=O)(=O)Cc4ccccc4)cc4ccccc34)c2n1]

### Step-1: Synthesis of tert-butyl ((2-cyano-1-((3-((phenylmethyl)sulfonamido)naphthalen-1-yl)methyl)-1H-pyrrolo[2,3-b]pyridin-6-yl)methyl)carbamate

A solution of tert-butyl ((2-carbamoyl-1-((3-((phenylmethyl)sulfonamido)naphthalen-1-yl)methyl)-1H-pyrrolo[2,3-b]pyridin-6-yl)methyl)carbamate (0.19 g, 0.317 mmol) in DCM (8 mL) was cooled to 0° C. and to the solution was added triethylamine (0.096 g, 0.95 mmol), trifluoroacetic anhydride (0.1 g, 0.47 mmol). After stirring at RT for 2 h, the reaction mixture was diluted with DCM, washed with sat. NaHCO₃, dried over Na₂SO₄ and concentrated. The crude compound was purified by Combi flash chromatography using 0.5% methanol in DCM as eluent to yield the title compound 16a (0.1 g, 54.2%). LC-MS: 582.2 [M+H]⁺.

### Step-2: Synthesis of tert-butyl (Z)-((2-(N′-hydroxycarbamimidoyl)-1-((3-((phenylmethyl)sulfonamido)naphthalen-1-yl)methyl)-1H-pyrrolo[2,3-b]pyridin-6-yl)methyl)carbamate

To a solution of tert-butyl ((2-cyano-1-((3-((phenylmethyl)sulfonamido)naphthalen-1-yl)methyl)-1H-pyrrolo[2,3-b]pyridin-6-yl)methyl)carbamate (0.1 g, 0.172 mmol) in THF (4 mL), Ethanol (4 mL) was added NH2OH·HCl (0.06 g, 0.86 mmol) and stirred at RT for 12 h. The reaction mixture was then poured on to ice water, the precipitate formed was filtered and dried to yield the title compound 16b (0.105 g, crude). LC-MS: 615.05 [M+H]⁺.

### Step-3: Synthesis of tert-butyl ((2-(5-hydroxy-1,2,4-oxadiazol-3-yl)-1-((3-((phenylmethyl)sulfonamido)naphthalen-1-yl)methyl)-1H-pyrrolo[2,3-b]pyridin-6-yl)methyl)carbamate

A solution of tert-butyl (Z)-((2-(N′-hydroxycarbamimidoyl)-1-((3-((phenylmethyl) sulfonamido)naphthalen-1-yl)methyl)-1H-pyrrolo[2,3-b]pyridin-6-yl)methyl)carbamate (0.05 g, 0.081 mmol) in THF (3 mL) was cooled to 0° C. and to the solution was added triphosgene (0.048 g, 0.16 mmol) and stirred for 1 h at the same temperature. The reaction mixture was gradually warmed to RT and stirred for 18 h at RT. The reaction mixture was diluted with ethyl acetate and quenched with NH₄Cl. The organic portion was washed with NH₄Cl, brine, dried over Na₂SO₄ and concentrated. This afforded the title compound 16c (0.051 g). LC-MS: 641.1 [M+H]⁺.

### Step-4: Synthesis of N-(4-((6-(aminomethyl)-2-(5-hydroxy-1,2,4-oxadiazol-3-yl)-1H-pyrrolo[2,3-b]pyridin-1-yl)methyl)naphthalen-2-yl)-1-phenylmethanesulfonamide

A solution of tert-butyl ((2-(5-hydroxy-1,2,4-oxadiazol-3-yl)-1-((3-((phenylmethyl)sulfonamido)naphthalen-1-yl)methyl)-1H-pyrrolo[2,3-b]pyridin-6-yl)methyl)carbamate (0.05 g, 0.078 mmol) in DCM was cooled to 0° C.′ and to the solution was added TFA (2 mL). The mixture was then stirred at RT for 1.5 h and concentrated. This afforded the title compound (Compound 353) (0.05 g, 98.4%). LC-MS: 541.2 [M+H]⁺; HNMR (CD₃OD, 400 MHz): δ 4.15 (s, 2H), 4.32 (s, 2H), 6.28 (s, 1H), 6.50 (s, 2H), 7.07-7.10 (m, 2H), 7.15-7.40 (m, 6H), 7.55-7.60 (m, 2H), 7.72-7.80 (m, 1H), 8.19-8.23 (m, 2H).

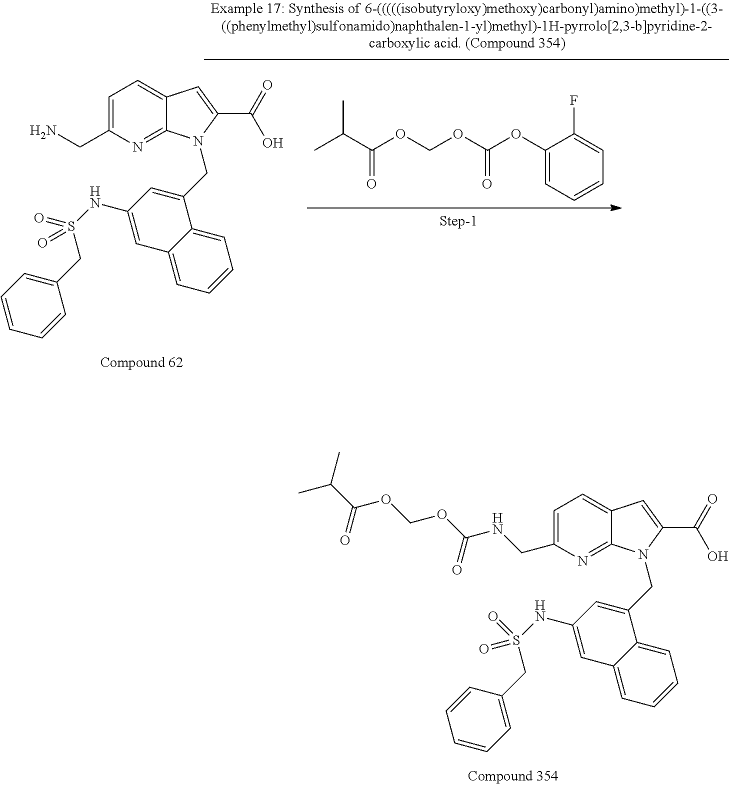 [SMILES: NCc1ccc2cc(C(=O)O)n(Cc3cc(NS(=O)(=O)Cc4ccccc4)cc4ccccc34)c2n1 ; CC(C)C(=O)OCOC(=O)Oc1ccccc1F ; "Step-1" ; CC(C)C(=O)OCOC(=O)NCc1ccc2cc(C(=O)O)n(Cc3cc(NS(=O)(=O)Cc4ccccc4)cc4ccccc34)c2n1]

### Step-1: Synthesis of 6-(((((isobutyryloxy)methoxy)carbonyl)amino)methyl)-1-((3-((phenylmethyl) sulfonamido)naphthalen-1-yl)methyl)-1H-pyrrolo[2,3-b]pyridine-2-carboxylic acid. (Compound 354)

To a stirred solution of 6-(aminomethyl)-1-((3-((phenylmethyl)sulfonamido)naphthalen-1-yl)methyl)-1H-pyrrolo[2,3-b]pyridine-2-carboxylic acid (0.2 g, 0.39 mmol) and (((2-fluorophenoxy)carbonyl)oxy)methyl isobutyrate (0.122 g, 0.47 mmol) in DMF (4 mL), was added DIPEA (0.154 g, 1.99 mmol) and stirred for 16 h. The reaction mixture was concentrated to form a residue and to the residue was added cold water to form a precipitate that was filtered and dried. The crude compound was purified by preparative HPLC to afford the pure title compound (Compound 354) (0.06 g, 23.8%). (Prep HPLC method: Column-ZORBAX (150 mm×21 mm), flow rate-20 mL/min, mobile phase-A was 0.1% formic acid in water, B was acetonitrile, Gradient program-10% B at 0 minute, 30% B at 2 minutes, 80% B at 8 minutes). LC-MS: 645.3 [M+H]⁺; 3HNMR (CD₃OD, 400 MHz): δ 1.07-1.08 (d, 6H), 2.47 (m, 1H), 4.16 (s, 2H), 4.42-4.44 (d, 2H), 5.54 (s, 2H), 6.22 (s, 1H), 6.47 (s, 2H), 7.00-7.02 (d, 2H), 7.14-7.23 (m, 4H), 7.45 (s, 1H), 7.52-7.54 (m, 2H), 7.75-7.78 (m, 1H), 7.78-7.80 (d, 1H), 8.13-8.15 (d, 1H), 8.20-8.22 (m, 1H).

The following compounds listed in Table 5 were prepared by following the similar procedure as described above for Example-17 using appropriate reagents with suitable modifications known to the one skilled in the art.

TABLE 5

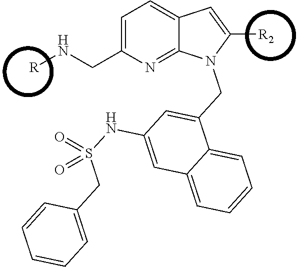

| Compound | R | R₂ | Spectral data |
|---|---|---|---|
| 355 | 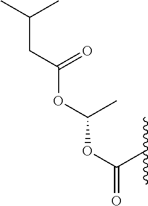 [SMILES: *C(=O)O[C@@H](C)OC(=O)CC(C)C] | —COOEt | LC-MS: 701.5 [M + H]⁺; ¹HNMR(CD₃OD, 400 MHz): δ 0.82-0.84 (m, 6H), 1.10-1.13 (t, 3H), 1.24-1.30 (d, 4H), 1.95-2.00 (m, 1H), 2.08-2.11 (m, 2H), 4.15-4.20 (m, 4H), 4.42(s, 2H) 6.23 (s, 1H), 6.45 (s, 2H), 6.64-6.68 (q, 1H), 7.00-7.02 (d, 2H), 7.13-7.25 (m, 3H), 7.47 (s, 1H), 7.52-7.55 (m, 3H), 7.79-7.81 (m, 1H), 8.15-8.17 (d, 1H), 8.21-8.23 (m, 1H). |
| 356 | 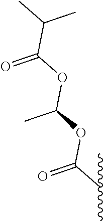 [SMILES: *C(=O)O[C@@H](C)OC(=O)C(C)C] | —COOH | LC-MS: 659.4 [M + H]⁺; ¹HNMR(CD₃OD, 400 MHz): δ 1.04-1.07 (t, 6H), 1.28-1.30 (d, 3H), 2.41-2.45 (m, 1H), 4.16(s, 2H), 4.40-4.42 (d, 2H), 6.21 (s, 1H), 6.47 (s, 2H), 6.47-6.64 (q, 1H), 7.01- 7.02(d, 2H), 7.01-7.23 (m, 3H), 7.45(s, 1H), 7.52- 7.53(m, 3H), 7.61(t, 1H), 7.78-7.80(d, 1H), 8.13- 8.15(d, 1H), 8.20-8.22 (d, 1H). |

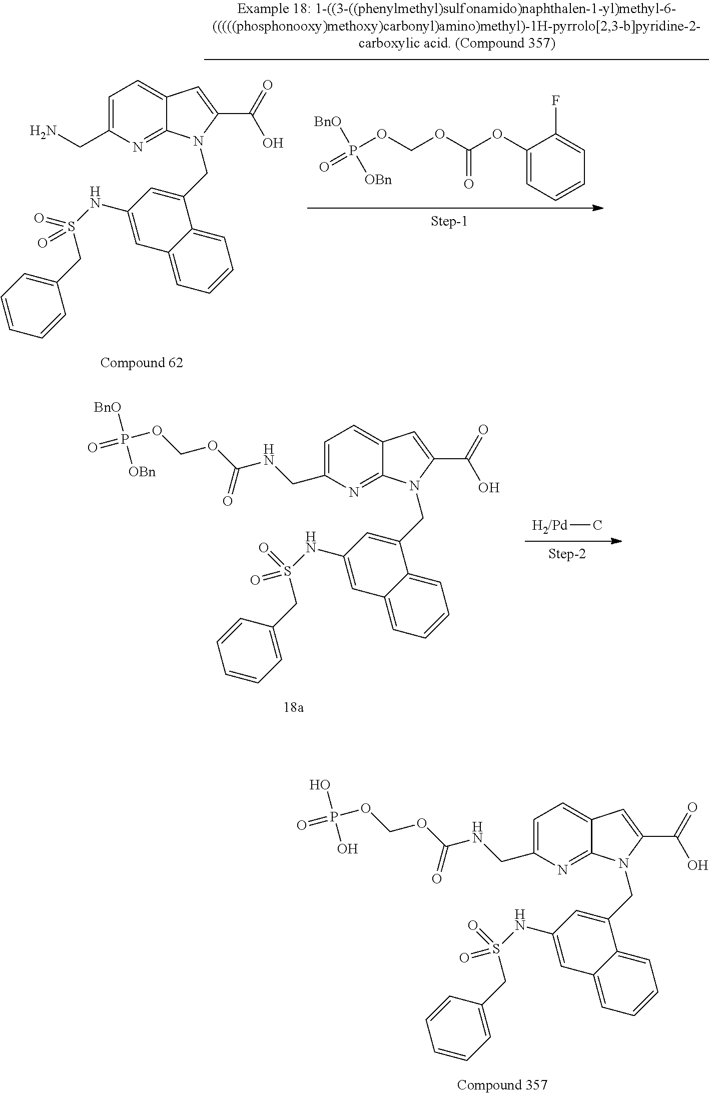

### Step-1: Synthesis of 6-((((((bis(benzyloxy)phosphoryl)oxy)methoxy)carbonyl)amino)methyl)-1-((3-((phenylmethyl)sulfonamido)naphthalen-1-yl)methyl)-1H-pyrrolo[2,3-b]pyridine-2-carboxylic acid

The experimental procedure was the same as described in Step-1 of Example-17 with appropriate variations in coupling methods, reactants, quantities of reagents, and solvents to obtain the compound 18a. LC-MS: 835.40 [M+H]⁺.

### Step-2:1-(3-((phenylmethyl)sulfonamido)naphthalen-1-yl)methyl)-6-(((((phosphonooxy) methoxy)carbonyl)amino)methyl)-1H-pyrrolo[2,3-b]pyridine-2-carboxylic acid. (Compound 357)

A suspension of Pd—C (10% load) (0.178 g, 1.67 mmol), 6-((((((bis(benzyloxy) phosphoryl)oxy)methoxy)carbonyl)amino)methyl)-1-((3-((phenylmethyl)sulfonamido) naphthalen-1-yl)methyl)-1H-pyrrolo[2,3-b]pyridine-2-carboxylic acid (0.14 g, 0.16 mmol) in methanol was stirred under positive pressure of hydrogen using a bladder at RT for 72 h. The reaction mixture was filtered through Celite, washed with 10% methanol in DCM. The filtrate was concentrated, and the residue was purified by preparative HPLC to get the pure title compound (Compound 357) (0.0055 g, 4.5%). (Prep HPLC method: Column-ZORBAX (150 mm×18 mm), flow rate-20 mL/min, mobile phase-A was 0.1% formic acid in water, B was acetonitrile, Gradient program-20% B at 0 minute, 30% B at 2 minutes, 60% B at 8 minutes). LC-MS: 655.2 [M+H]⁺; ¹HNMR (CD₃OD, 400 MHz): δ 4.19 (s, 2H), 4.25 (s, 4H), 5.39-5.42 (d, 2H), 6.21 (s, 1H), 6.48 (s, 2H), 7.01-7.02 (s, 2H), 7.15-7.28 (m, 4H), 7.47 (s, 1H), 7.53-7.55 (m, 3H), 7.78-7.80 (m, 1H), 8.15-8.17 (d, 1H), 8.21-8.23 (d, 1H).

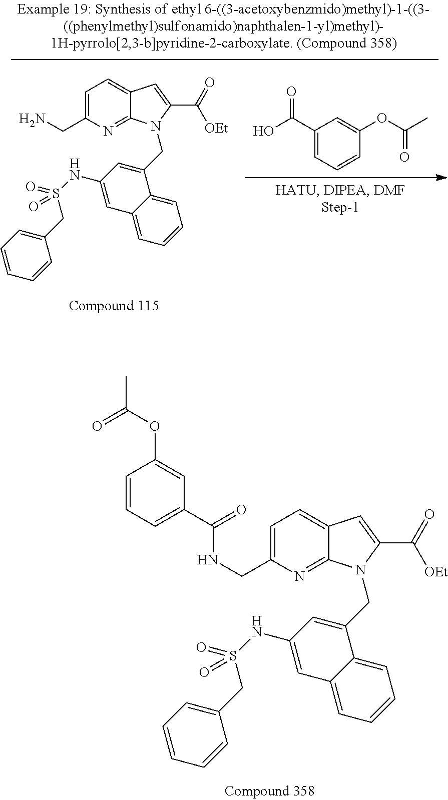

### Step-1:

The experimental procedure was the same as described in Step-8 of Example-2 with appropriate variations in coupling methods, reactants, quantities of reagents, and solvents (0.041 g, 15.6%). LC-MS: 705.3 [M+H]⁺; ¹HNMR (CD₃OD, 400 MHz): δ 1.12-1.15 (t, 3H), 1.91 (s, 3H), 4.06 (s, 2H), 4.17-4.22 (q, 2H), 4.70 (s, 2H), 5.12 (s, 2H), 6.23 (s, 1H), 6.5 (s, 2H), 6.84-6.88 (m, 1H), 6.97-6.99 (d, 2H), 7.12-7.7.16 (t, 2H), 7.20-7.21 (d, 2H), 7.30-7.34 (m, 3H), 7.50-7.57 (m, 4H), 7.82-7.84 (d, 1H), 8.18-8.23 (d, 2H).

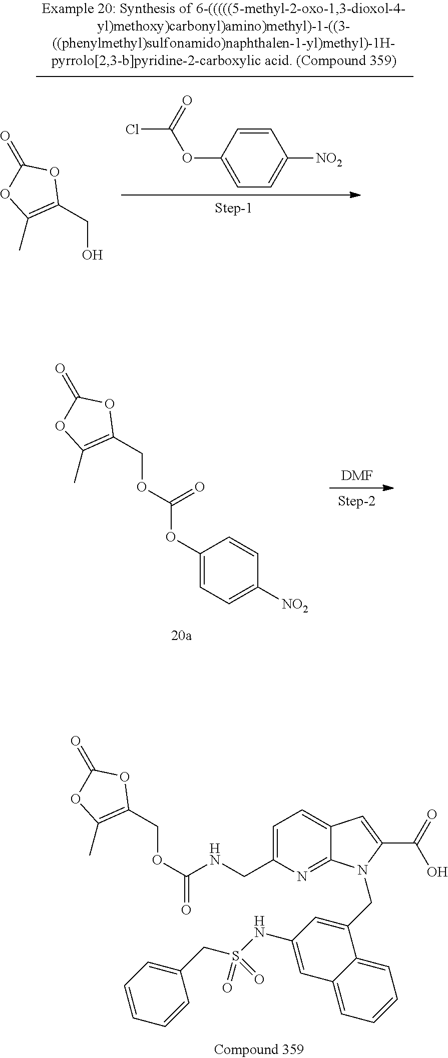

### Step-1: Synthesis of (5-methyl-2-oxo-1,3-dioxol-4-yl)methyl (4-nitrophenyl) carbonate

A solution of 4-(hydroxymethyl)-5-methyl-1,3-dioxol-2-one in Chloroform was cooled to 0° C. and to the solution was added pyridine (0.109 g, 1.38 mmol) followed by dropwise addition of 4-nitrophenyl carbonochloridate (0.256 g, 1.26 mmol). The reaction mixture was stirred at RT for 20 h, then diluted with DCM, washed with 1% NaOH solution, followed by washing with 1N HCL. The organic portion was dried over Na₂SO₄ and concentrated. The residue was washed with (1:1) hexane-diethyl ether to get the pure title compound 20a (0.15 g, 44.07%), ¹HNMR (CDCl₃, 400 MHz): δ 2.22 (s, 3H), 5.05 (s, 2H), 7.37-7.51 (m, 2H), 8.27-8.36 (m, 2H).

### Step-2: Synthesis of 6-(((((5-methyl-2-oxo-1,3-dioxol-4-yl)methoxy)carbonyl)amino)methyl)-1-((3-((phenylmethyl)sulfonamido)naphthalen-1-yl)methyl)-1H-pyrrolo[2,3-b]pyridine-2-carboxylic acid. (Compound 359)

A mixture of (5-methyl-2-oxo-1,3-dioxol-4-yl)methyl (4-nitrophenyl) carbonate (0.13 g, 0.4 mmol) and 6-(aminomethyl)-1-((3-((phenylmethyl)sulfonamido)naphthalen-1-yl)methyl)-1H-pyrrolo[2,3-b]pyridine-2-carboxylic acid (0.2 g, 0.4 mmol) was stirred at RT for 16 h. Then the reaction mixture was poured into water, the solid separated was filtered and dried. The crude compound was purified by preparative HPLC to get the pure title compound (Compound 359) (0.041 g, 15.6%). LC-MS: 657.30 [M+H]⁺; ¹HNMR (CD₃OD, 400 MHz): δ 2.05 (s, 3H), 4.17 (s, 2H), 4.43 (s, 2H), 4.68 (s, 2H), 6.22 (s, 1H), 6.50 (s, 2H), 7.00-7.02 (d, 2H), 7.14-7.18 (m, 3H), 7.20-7.24 (m, 2H), 7.44 (s, 1H), 7.55 (m, 3H), 7.79-7.81 (m, 1H), 8.14-8.16 (d, 1H), 8.22-8.25 (d, 1H).

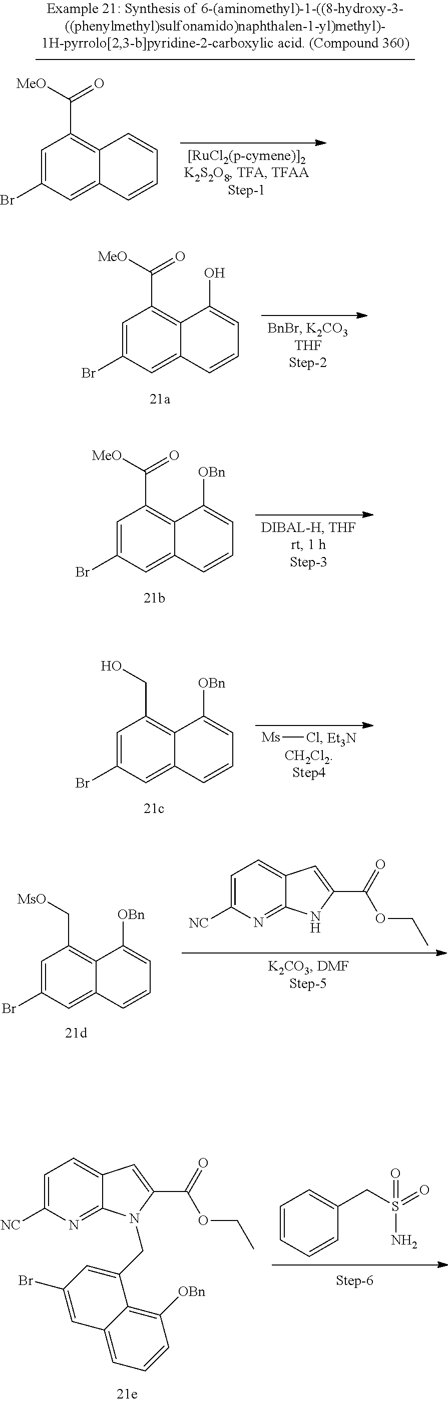

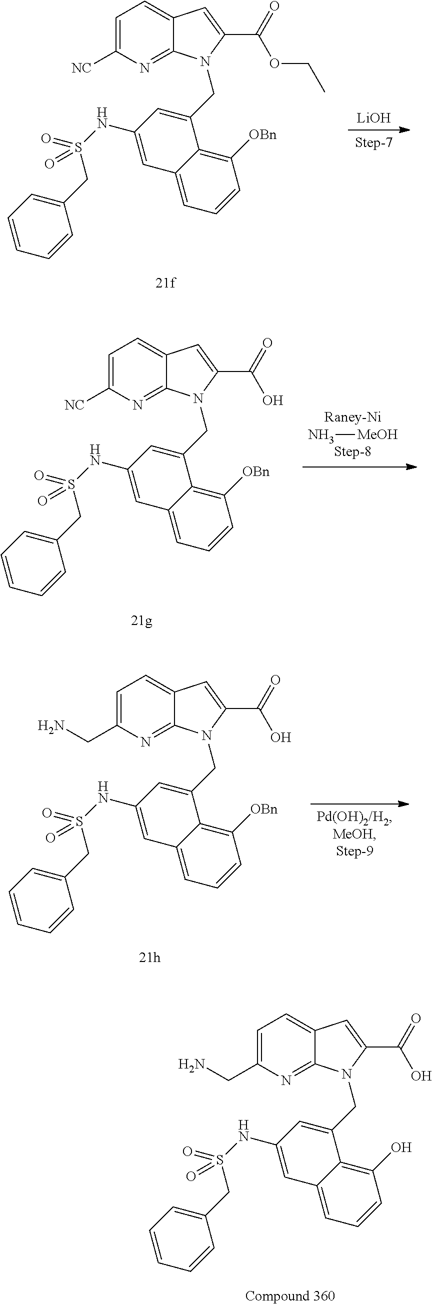

### Step-1: Synthesis of methyl 3-bromo-8-hydroxy-1-naphthoate

A mixture of [RuCl₂(p-cymene)]₂ (275 mg, 0.449 mmol), K₂S₂O₈ (5.324 g, 19.6 mmol), and methyl 3-bromo-1-naphthoate (5.0 g, 17.9 mmol) in trifluoroacetic acid 112 mL) and trifluoroacetic anhydride (50 mL) taken in a sealed tube was heated to 70° C. for 16 h. The reaction mixture was cooled to RT and quenched with water, extracted with DCM, washed with saturated ammonium chloride solution. The organic portion was dried over Na₂SO₄ and concentrated. The residue was purified by combi flash column chromatography by eluting with 40% ethyl acetate in hexane to yield the title compound 21a as a yellow solid (2.2 g). LC-MS: 280.2 [M+H]⁺.

### Step 2: methyl 8-(benzyloxy)-3-bromo-1-naphthoate

To a stirred solution of methyl 3-bromo-8-hydroxy-1-naphthoate (2.1 g, 7.1 mmol) in DMF (S mL) was added Cs₂CO₃ (6.95 mg, 21.3 mmol) at 0° C. followed by benzyl bromide (1.0 mL, 8.5 mmol) and the reaction was stirred for overnight. The reaction mixture was added ice-cold water and extracted with ethyl acetate. The extracts were washed with saturated aqueous NaCl, dried over Na₂SO₄, and concentrated under reduced pressure. The residue was purified by combi flash column chromatography, eluting with 40% ethyl acetate in hexane to yield the title compound 21b as a yellow solid (1.9 g).

### Step 3: (8-(benzyloxy)-3-bromonaphthalen-1-yl)methanol

A solution of methyl 8-(benzyloxy)-3-bromo-1-naphthoate (1.9 g, 4.93 mmol) in THF (50 mL) was cooled to 0° C. and to the solution was added DIBAL-H (55 mL, 49.3 mmol) and the reaction was stirred for overnight. After the completion of the reaction. Sat. Potassium sodium L-tartrate (2 mL) was added at 0° C. and the mixture was extracted with ethyl acetate. The extracts were washed with saturated aqueous NaCl, dried over Na₂SO₄, and concentrated under reduced pressure. The residue was purified by combi flash column chromatography, eluting with 40% ethyl acetate in hexane to give the title compound 21c as a yellow solid (1.2 g). LCMS: 343.1 [M+H]⁺.

### Step 4: (8-(benzyloxy)-3-bromonaphthalen-1-yl)methyl methanesulfonate

A stirred solution of (8-(benzyloxy)-3-bromonaphthalen-1-yl)methanol (0.5 g, 1.45 mmol) in DCM (10 mL) was cooled to 0° C. and to the solution was added triethylamine (0.6 mL, 4.37 mmol), followed by MsCl (0.16 mL, 2.18 mmol) and the reaction was stirred for 2 h. Then Reaction mixture was added water and extracted with ethyl acetate The extracts were washed with saturated aqueous NaCl, dried over Na₂SO₄ and concentrated. The residue was purified by combi flash column chromatography, eluting with 40% ethyl acetate in hexane to yield the title compound 21d as a yellow solid (0.68 g). LCMS: 419.20 [M−H]⁻.

### Step-5: Synthesis of ethyl 1-((8-(benzyloxy)-3-bromonaphthalen-1-yl)methyl)-6-cyano-1H-pyrrolo[2,3-b]pyridine-2-carboxylate

The experimental procedure was the same as described in Step-1 of Example-1 with appropriate variations in coupling methods, reactants, quantities of reagents, and solvents to obtain the compound 21e. LCMS: 540.20 [M+H]⁺.

### Step-6: Synthesis of ethyl 1-(8-(benzyloxy)-3-((phenylmethyl)sulfonamido)naphthalen-1-yl)methyl)-6-cyano-1H-pyrrolo[2,3-b]pyridine-2-carboxylate

The experimental procedure was the same as described in Step-2 of Example-1 with appropriate variations in coupling methods, reactants, quantities of reagents, and solvents to obtain the compound 21f. LCMS: 631.30 [M+H]⁺.

### Step-7: Synthesis of 1-(8-(benzyloxy)-3-(phenylmethyl)sulfonamido)naphthalen-1-yl)methyl)-6-cyano-1H-pyrrolo[2,3-b]pyridine-2-carboxylic acid

The experimental procedure was the same as described in Step-3 of Example-1 with appropriate variations in coupling methods, reactants, quantities of reagents, and solvents to obtain the compound 21g. LCMS: 603.30 [M+H]⁺.

### Step-8: Synthesis of 6-(aminomethyl)-1-((8-(benzyloxy)-3-((phenylmethyl)sulfonamido)naphthalen-1-yl)methyl)-1H-pyrrolo[2,3-b]pyridine-2-carboxylic acid

The experimental procedure was the same as described in Step-4 of Example-1 with appropriate variations in coupling methods, reactants, quantities of reagents, and solvents to obtain the compound 21h. LCMS: 607.30 [M+H]⁺.

### Step-9: Synthesis of 6-(aminomethyl)-1-((8-hydroxy-3-((phenylmethyl)sulfonamido)naphthalen-1-yl)methyl)-1H-pyrrolo[2,3-b]pyridine-2-carboxylic acid. (Compound 360)

A suspension of Pd(OH)₂ (100 mg) in methanol (6 mL) was added 6-(aminomethyl)-1-((8-(benzyloxy)-3-((phenylmethyl)sulfonamido)naphthalen-1-yl)methyl)-1H-pyrrolo[2,3-b]pyridine-2-carboxylic acid (0.085 g, 0.14 mmol) at RT. The mixture was stirred under a positive pressure of hydrogen using a bladder for 6 h. The reaction mixture was filtered through a Celite pad and the filtrate was evaporated. The was triturated with 5% diethyl ether in pentane and dried. This was further purified by preparative HPLC afforded the title compound (Compound 360) (0.05 g, 69.4%). (Preparative HPLC Method: Column: LUNA OMEGA (250×21.2 mm) 5μ; Mobile Phase A: 0.05% TFA in Water, B: Acetonitrile; Flow rate: 18 ml/min; % B—10 at 0 minute, 20 at 2 minutes, 60 at 10 minutes). LC-MS: 517.30 [M+H]⁺; ¹HNMR (CD₃OD, 400 MHz): δ 4.14 (s, 2H), 4.30 (s, 2H), 5.96 (d, 1H), 6.80-6.85 (d, 1H), 6.85 (s, 1H), 7.061-7.066 (dd, 2H), 7.18-7.28 (m, 7H), 7.50 (s, 1H), 8.23-8.25 (d, 1H).

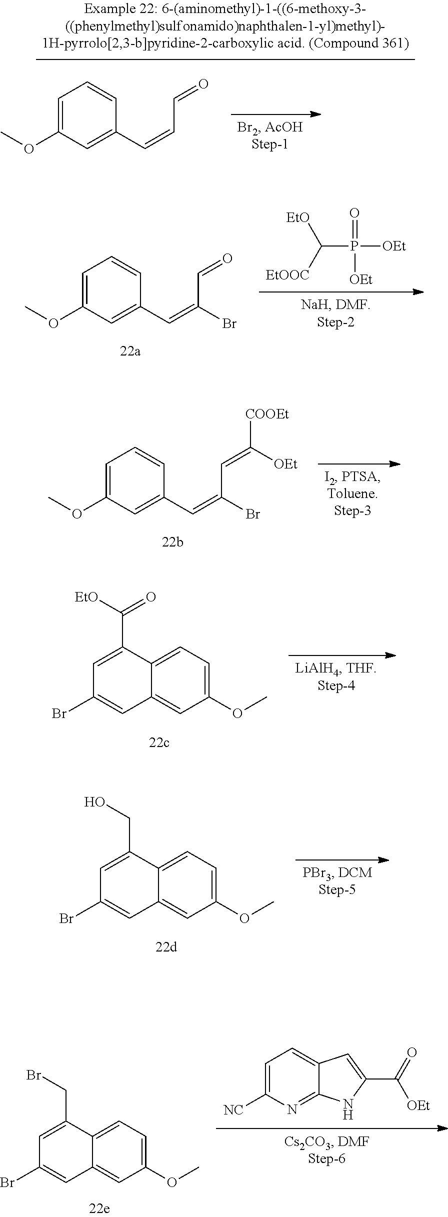

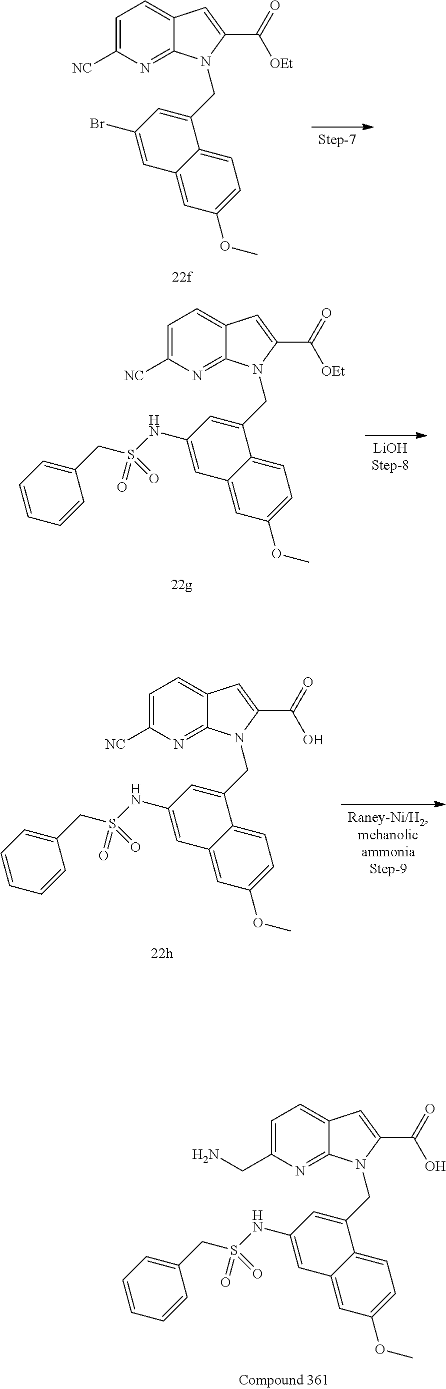

### Step-1: Synthesis of (E)-2-bromo-3-(3-methoxyphenyl) acrylaldehyde

To a stirred solution of (Z)-3-(3-methoxyphenyl) acrylaldehyde (1.5 g, 9.25 mmol) in acetic acid (3.7 mL) was added bromine ((0.47 mL, 9.25 mmol). After stirring the reaction mixture at RT for 2 h, the reaction mixture was added Aqueous NaHSO3 and triethylamine (1.28 mL, 9.25 mmol) at 0° C. After completion of addition, the reaction mixture was stirred at 60° C. for 4 h, cooled to RT and poured into ice-cold water, extracted with ethyl acetate, the organic portion was dried over Na₂SO₄ and concentrated. The crude compound was purified in combi flash chromatography using 10% ethyl acetate in hexane as eluent to afford pure title compound 22a (0.98 g). LC-MS: 243.0 [M+2]⁺.

### Step-2: Synthesis of ethyl (2Z,4E)-4-bromo-2-ethoxy-5-(3-methoxyphenyl) penta-2,4-dienoate

A stirred suspension of NaH (0.225 g, 5.62 mmol) in DMF (10 mL) was added ethyl 2-(diethoxyphosphoryl)-2-ethoxyacetate (1.5 g, 5.62 mmol) and stirred for 30 minutes. Then added (Z)-3-(3-methoxyphenyl) acrylaldehyde (0.9 g, 3.75 mmol), resulting mixture was stirred at RT for 16 h. The reaction mixture was poured into water, extracted with ethyl acetate, the organic portion was dried over Na₂SO₄ and concentrated. The crude compound was purified by combi flash chromatography using 10% ethyl acetate in hexane as eluent to afford the pure title compound 22b (0.89 g). LC-MS: 352.9 [M+H]⁺.

### Step-3: Synthesis of ethyl 3-bromo-6-methoxy-1-naphthoate

To a stirred solution of ethyl (2Z,4E)-4-bromo-2-ethoxy-5-(3-methoxyphenyl) penta-2,4-dienoate (0.89 g, 2.5 mmol) was added iodine (0.013 g, 0.05 mmol) and p-toluene sulfonic acid monohydrate (1.19 g, 6.27 mmol). The resulting mixture was stirred at 120° C. for 2 h. The reaction mixture was cooled to RT, added water, extracted with ethyl acetate, the organic portion was dried over Na₂SO₄ and concentrated. The crude was purified on combi flash chromatography using 15% ethyl acetate in hexane as eluent to afford pure title compound 22c (0.35 g). LC-MS: 309.90 [M+H]⁺.

### Step-4: Synthesis of (3-bromo-6-methoxynaphthalen-1-yl)methanol

A solution of ethyl 3-bromo-6-methoxy-1-naphthoate (0.32 g, 0.9 mmol) in THE (5 mL) was cooled to 0° C. and to the solution was added LiAlH4 (0.9 mL, 2M, 1.8 mmol). After addition, the reaction mixture was gradually warmed to RT and stirred for 30 minutes. The reaction mixture was quenched with ice water, stirred for 5 minutes. Solid separated was filtered and the filtrate was concentrated. The crude compound was purified by combi flash chromatography using 40% ethyl acetate in hexane as eluent. This afforded the pure title compound 22d (0.15 g). LC-MS: 266.8 [M−H]⁻.

### Step-5: Synthesis of 3-bromo-1-(bromomethyl)-6-methoxynaphthalene

A solution of (3-bromo-6-methoxynaphthalen-1-yl)methanol (0.15 g, 0.557 mmol) in DCM (3 mL) was cooled to 0° C. and to the solution was added PBr₃ (0.037 mL, 0.39 mmol). The reaction mixture was then stirred at RT for 1 h, then quenched with Aqueous NaHCO₃ solution, extracted with DCM, the organic portion was dried over Na₂SO₄ and concentrated to yield the title compound 22e (0.096 g). LC-MS: 329.8 [M−H]⁻.

### Step-6: Synthesis of ethyl 1-(3-bromo-6-methoxynaphthalen-1-yl)methyl)-6-cyano-1H-pyrrolo[2,3-b]pyridine-2-carboxylate

The experimental procedure was the same as described in Step-1 of Example-1 with appropriate variations in coupling methods, reactants, quantities of reagents, and solvents to obtain the compound 22f. LC-MS: 465.0 [M+2]⁺.

### Step-7: Synthesis of ethyl 6-cyano-1-(6-methoxy-3-((phenylmethyl)sulfonamido)naphthalen-1-yl)methyl)-1H-pyrrolo[2,3-b]pyridine-2-carboxylate

The experimental procedure was the same as described in Step-2 of Example-1 with appropriate variations in coupling methods, reactants, quantities of reagents, and solvents to obtain the compound 21g. LC-MS: 555.30 [M+H]⁺.

### Step-8: Synthesis of 6-cyano-1-((6-methoxy-3-((phenylmethyl)sulfonamido)naphthalen-1-yl)methyl)-1H-pyrrolo[2,3-b]pyridine-2-carboxylic acid

The experimental procedure was the same as described in Step-3 of Example-1 with appropriate variations in coupling methods, reactants, quantities of reagents, and solvents to obtain the compound 21h. LCMS: 527.2 [M+H]⁺.

### Step-9: Synthesis of 6-(aminomethyl)-1-(6-methoxy-3-((phenylmethyl)sulfonamido)naphthalen-1-yl)methyl)-1H-pyrrolo[2,3-b]pyridine-2-carboxylic acid. (Compound 361)

The experimental procedure is the same as described in Step-9 of Example-21 with appropriate variations in coupling methods, reactants, quantities of reagents, and solvents to obtain the compound 22 (compound 361).

LC-MS: 531.30 [M+H]⁺; ¹HNMR (CD₃OD, 400 MHz): δ 3.93 (s, 3H), 4.19 (s, 2H), 4.32 (s, 2H), 6.07 (s, 1H), 6.52 (s, 2H), 7.07-7.08 (d, 2H), 7.17 (s, 1H), 7.20-7.21 (d, 1H), 7.23-7.28 (m, 4H), 7.39 (s, 1H), 7.51 (s, 1H), 8.12-8.14 (d, 1H), 8.24-8.26 (d, 1H).

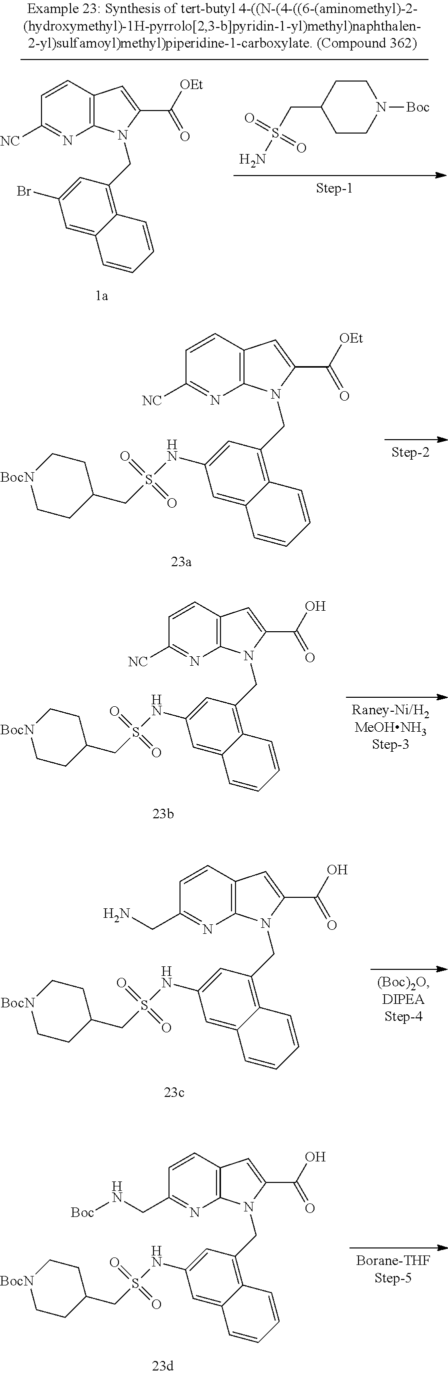

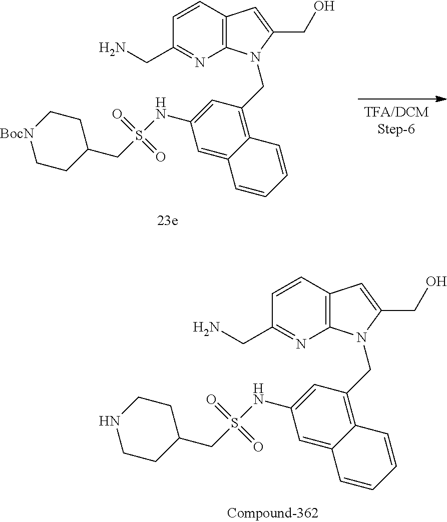 [SMILES: CC(C)(C)OC(=O)N1CCC(CS(=O)(=O)Nc2cc(Cn3c(CO)cc4ccc(CN)nc43)c3ccccc3c2)CC1 ; "TFA/DCMStep-6" ; NCc1ccc2cc(CO)n(Cc3cc(NS(=O)(=O)CC4CCNCC4)cc4ccccc34)c2n1]

### Step-1: Synthesis of ethyl 1-((3-(((1-(tert-butoxycarbonyl) piperidin-4-yl)methyl)sulfonamido)naphthalen-1-yl)methyl)-6-cyano-1H-pyrrolo[2,3-b]pyridine-2-carboxylate

The experimental procedure was the same as described in Step-2 of Example-1 with appropriate variations in coupling methods, reactants, quantities of reagents, and solvents to obtain the compound 23a (0.24 g).

### Step-2: Synthesis of 1-(3-(((1-(tert-butoxycarbonyl) piperidin-4-yl)methyl)sulfonamido)naphthalen-1-yl)methyl)-6-cyano-1H-pyrrolo[2,3-b]pyridine-2-carboxylic acid

The experimental procedure was the same as described in Step-3 of Example-1 with appropriate variations in coupling methods, reactants, quantities of reagents, and solvents to obtain the compound 23b (0.2 g). LC-MS: 602.30 [M−H]⁻.

### Step-3: Synthesis of 6-(aminomethyl)-1-((3-(((1-(tert-butoxycarbonyl) piperidin-4-yl)methyl)sulfonamido)naphthalen-1-yl)methyl)-1H-pyrrolo[2,3-b]pyridine-2-carboxylic acid

The experimental procedure was the same as described in Step-4 of Example-1 with appropriate variations in coupling methods, reactants, quantities of reagents, and solvents to obtain the compound 23c (0.18 g). LC-MS: 608.40 [M+H]⁺.

### Step-4: Synthesis of 6-(((tert-butoxycarbonyl)amino methyl)-1-(3-(1-(tert-butoxycarbonyl) piperidin-4-yl)methyl)sulfonamido)naphthalen-1-yl)methyl)-1H-pyrrolo[2,3-b]pyridine-2-carboxylic acid

A solution of 6-(aminomethyl)-1-((3-(((1-(tert-butoxycarbonyl) piperidin-4-yl)methyl)sulfonamido)naphthalen-1-yl)methyl)-1H-pyrrolo[2,3-b]pyridine-2-carboxylic acid (0.15 g, 0.24 mmol) in THF (4 mL), water (4 mL) was cooled to 0° C. and to the solution were added DMAP (0.012 g, 0.098 mmol) and DIPEA (0.159 g, 1.23 mmol) followed by Boc anhydride (0.107 g, 0.49 mmol) and stirred at RT for 12 h. The reaction mixture was diluted with brine solution, extracted with DCM, organic portion was dried over Na₂SO₄ and concentrated to yield the title compound to obtain the compound 23d (0.2 g). LC-MS: 708.40 [M+H]⁺.

### Step-5: Synthesis of tert-butyl 4-(N-(4-(6-(aminomethyl)-2-(hydroxyethyl)-1H-pyrrolo[2,3-b]pyridin-1-yl)methyl)naphthalen-2-yl) sulfamoyl)methyl) piperidine-1-carboxylate

A solution of 6-(aminomethyl)-1-((3-(((1-(tert-butoxycarbonyl) piperidin-4-yl)methyl)sulfonamido)naphthalen-1-yl)methyl)-1H-pyrrolo[2,3-b]pyridine-2-carboxylic acid (0.2 g, 0.2 mmol) in THF (10 mL) was cooled to 0° C. and to the solution was added Borane-THF (0.04 g, 0.5 mmol). After completion of addition, the reaction mixture was stirred for 12 h at RT. The mixture was then cooled again to 0° C. and quenched with methanol. After stirring for 2 h, to the reaction mixture was added water, extracted with DCM, washed with brine, and the organic portion was dried over Na₂SO₄ and concentrated to yield the title compound 25e (0.15 g). LC-MS: 694.40 [M+H]⁺.

### Step-6: Synthesis of ter-butyl 4-(N-(4-((6-(aminomethyl)-2-(hydroxymethyl)-1H-pyrrolo[2,3-b]pyridin-1-yl)methyl)naphthalen-2-yl)sulfamoyl)methyl)piperidine-1-carboxylate

The experimental procedure was the same as described in Step-5 of Example-1 with appropriate variations in coupling methods, reactants, quantities of reagents, and solvents to obtain the compound (Compound 362) (0.045 g). (Preparative HPLC method: Column-LUNA C18 (250 mm×21.2 mm), 5.0μ; Eluent-A was 0.1% TFA in water, B was acetonitrile (1:1), gradient-10% B at 0 minute, 20% B at 2 minutes, 50% at 10 minutes). LC-MS: 494.3 [M+H]⁺. ¹HNMR (CD₃OD, 400 MHz): δ 1.44-1.50 (m, 1H), 2.0-2.16 (m, 4H), 2.0-2.98 (m, 4H), 3.35 (s, 2H), 4.30-4.35 (d, 2H), 4.64 (s, 2H), 6.22-6.24 (d, 1H), 6.55 (s, 2H), 7.50-7.58 (m, 4H), 7.82-7.84 (m, 2H), 8.23-8.29 (m, 2H).

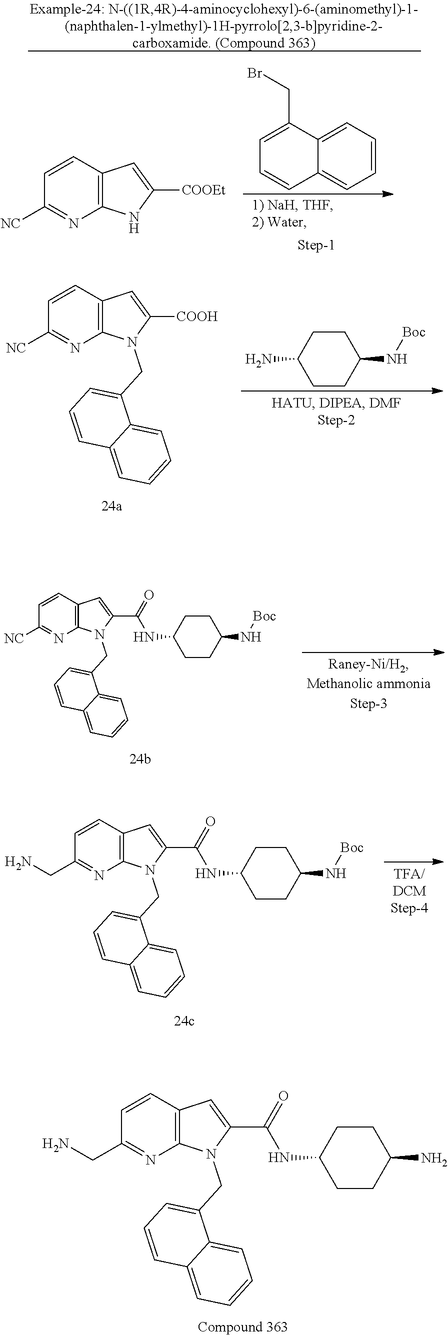

### Step-1: Synthesis of 6-cyano-1-(naphthalen-1-ylmethyl)-1H-pyrrolo[2,3-b]pyridine-2-carboxylic acid

A stirred solution of ethyl 6-cyano-1H-pyrrolo[2,3-b]pyridine-2-carboxylate (0.3 g, 1.39 mmol) in DMF (3 mL) was cooled to 0° C., added NaH (0.037 g, 1.53 mmol) and 1-(bromomethyl)naphthalene (0.308 g, 1.39 mmol). After stirring for 2 h at RT added ice-cold water and acidified with 1N HCl, extracted with ethyl acetate, washed with brine solution, dried over Na₂SO₄ and concentrated to obtain the compound 24a (0.35 g, 77.7%). LC-MS: 326.15 [M−H]⁻.

### Step-2: Synthesis of tert-butyl ((1R,4R)-4-(6-cyano-1-(naphthalen-1-ylmethyl)-1H-pyrrolo[2,3-b]pyridine-2-carboxamido)cyclohexyl)carbamate

The experimental procedure was the same as described in Step-9 of Example-1 with appropriate variations in coupling methods, reactants, quantities of reagents, and solvents to obtain the compound 24b (0.12 g). LC-MS: 524.35 [M+H]⁺.

### Step-3: Synthesis of tert-butyl ((1r,4r)-4-(6-(aminomethyl)-1-(naphthalen-1-ylmethyl)-1H-pyrrolo[2,3-b]pyridine-2-carboxamido)cyclohexyl)carbamate

The experimental procedure was the same as described in Step-10 of Example-1 with appropriate variations in coupling methods, reactants, quantities of reagents, and solvents to obtain the compound 24c (0.105 g). LCMS: 528.2 [M−H]⁻.

### Step-4: Synthesis of N-((1r,4r)-4-aminocyclohexyl)-6-(aminomethyl)-1-(naphthalen-1-ylmethyl)-1H-pyrrolo[2,3-b]pyridine-2-carboxamide

The experimental procedure was the same as described in Step-11 of Example-1 with appropriate variations in coupling methods, reactants, quantities of reagents, and solvents to obtain the title compound (Compound 363) (0.08 g). LC-MS: 428.0 [M+H]⁺; ¹HNMR (CD₃OD, 300 MHz): δ 1.26-1.42 (m, 4H), 1.78-1.99 (m, 4H), 2.95-3.05 (m, 1H), 3.55-3.65 (m, 1H), 4.35 (s, 2H), 6.35-6.38 (d, 1H), 6.50 (s, 2H), 7.13-7.28 (m, 3H), 7.50-7.89 (m, 4H), 8.18-8.22 (m, 2H).

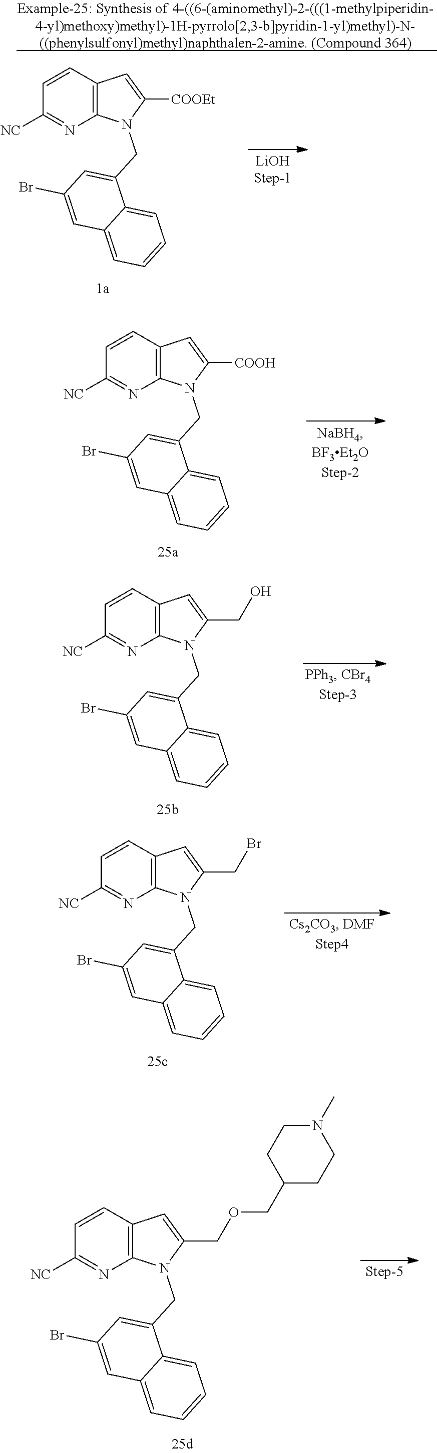 [SMILES: CCOC(=O)c1cc2ccc(C#N)nc2n1Cc1cc(Br)cc2ccccc12 ; N#Cc1ccc2cc(C(=O)O)n(Cc3cc(Br)cc4ccccc34)c2n1 ; N#Cc1ccc2cc(CO)n(Cc3cc(Br)cc4ccccc34)c2n1 ; N#Cc1ccc2cc(CBr)n(Cc3cc(Br)cc4ccccc34)c2n1 ; CN1CCC(COCc2cc3ccc(C#N)nc3n2Cc2cc(Br)cc3ccccc23)CC1]

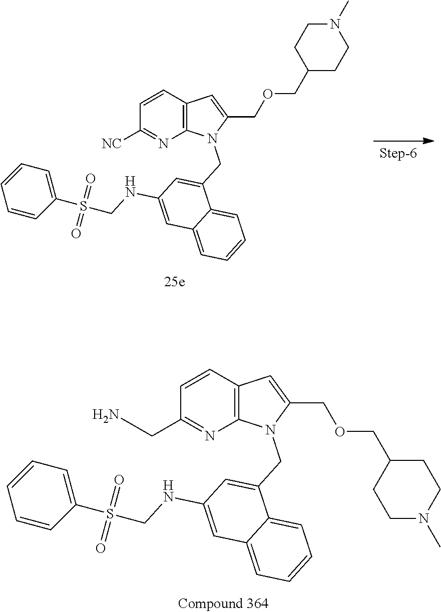 [SMILES: CN1CCC(COCc2cc3ccc(C#N)nc3n2Cc2cc(NCS(=O)(=O)c3ccccc3)cc3ccccc23)CC1 ; CN1CCC(COCc2cc3ccc(CN)nc3n2Cc2cc(NCS(=O)(=O)c3ccccc3)cc3ccccc23)CC1]

### Step-1: Synthesis of 1-((3-bromonaphthalen-1-yl)methyl)-6-cyano-1H-pyrrolo[2,3-b]pyridine-2-carboxylic acid

The experimental procedure was the same as described in Step-3 of Example-1 with appropriate variations in coupling methods, reactants, quantities of reagents, and solvents to obtain the compound 25a (1.3 g, 92.6%). LC-MS: 408.10 [M+2]⁺.

### Step-2: Synthesis of 1-(3-bromonaphthalen-1-yl)methyl)-2-(hydroxymethyl)-1H-pyrrolo[2,3-b]pyridine-6-carbonitrile

A solution of 1-((3-bromonaphthalen-1-yl)methyl)-6-cyano-1H-pyrrolo[2,3-b]pyridine-2-carboxylic acid (1 g, 2.46 mmol) in THF (15 mL) was cooled to 0° C. and to the solution was added NaBH₄ (0.23 g, 6.15 mmol) followed by BF3-etherate. After the addition, the reaction mixture was warmed to RT and stirred for 2 h. The mixture was then quenched with Aqueous Saturated Na₂SO₄ solution and filtered, and the filtrate was dried over Na₂SO₄ and concentrated. The crude compound was purified by combi flash chromatography using 40% ethyl acetate in hexane as eluent. This afforded the pure title compound 25b (0.45 g, 46.6%). LC-MS: 394.0 [M+2]⁺.

### Step-3: Synthesis of 2-(bromomethyl)-1-((3-bromonaphthalen-1-yl)methyl)-1H-pyrrolo[2,3-b]pyridine-6-carbonitrile

A solution of 1-((3-bromonaphthalen-1-yl)methyl)-2-(hydroxymethyl)-1H-pyrrolo[2,3-b]pyridine-6-carbonitrile (0.43 g, 1.09 mmol) in DCM (10 mL) was cooled to 0° C.), added triphenylphosphine (0.43 g, 1.64 mmol) and CBr4 (0.54 g, 1.64 mmol), stirred at RT for 2 h and concentrated. The crude was purified by combi flash chromatography using 30% ethyl acetate in hexane as eluent. This afforded the pure title compound 25c (0.33 g, 66.15%). LC-MS: 456.1 [M+H]⁺.

### Step-4: Synthesis of 1-((3-bromonaphthalen-1-yl)methyl)-2-(((1-methylpiperidin-4-yl)methoxy)methyl)-1H-pyrrolo[2,3-b]pyridine-6-carbonitrile

The experimental procedure was the same as described in Step-1 of Example-1 with appropriate variations in coupling methods, reactants, quantities of reagents, and solvents to obtain the compound 25d (0.2 g, 58.4%). LC-MS: 505.2 [M+2]⁺.

### Step-5: Synthesis of 2-(((1-methylpiperidin-4-yl)methoxy)methyl)-1-((3-(((phenylsulfonyl)methyl)amino)naphthalen-1-yl)methyl)-1H-pyrrolo[2,3-b]pyridine-6-carbonitrile

The experimental procedure was the same as described in Step-2 of Example-1 with appropriate variations in coupling methods, reactants, quantities of reagents, and solvents to obtain the compound 25e (0.1 g, 47.5%). LC-MS: 594.40 [M+H]⁺.

### Step-6: Synthesis of 4-((6-(aminomethyl)-2-(((1-methylpiperidin-4-yl)methoxy)methyl)-1H-pyrrolo[2,3-b]pyridin-1-yl)methyl)-N-((phenylsulfonyl)methyl)naphthalen-2-amine

The experimental procedure was the same as described in Step-7 of Example-1 with appropriate variations in coupling methods, reactants, quantities of reagents, and solvents to obtain the title compound ((Compound 364) (0.03 g, 39.8%). LC-MS: 598.40 [M+H]⁺; ¹HNMR (CD₃OD, 400 MHz): δ 1.15-1.25 (m, 2H), 1.42 (s, 2H), 1.57-1.60 (d, 2H), 2.65-2.75 (m, 5H), 3.23-3.24 (d, 2H), 3.28 (s, 1H), 4.21 (s, 2H), 4.28 (s, 2H), 4.64 (s, 2H), 6.17 (s, 2H), 6.42 (s, 1H), 6.72 (s, 1H), 7.11-7.13 (d, 2H), 7.21-7.31 (m, 4H), 7.43 (s, 1H), 7.55-7.59 (m, 2H), 7.81-7.84 (m, 1H), 8.09-8.11 (d, 1H), 8.20-8.22 (t, 1H).

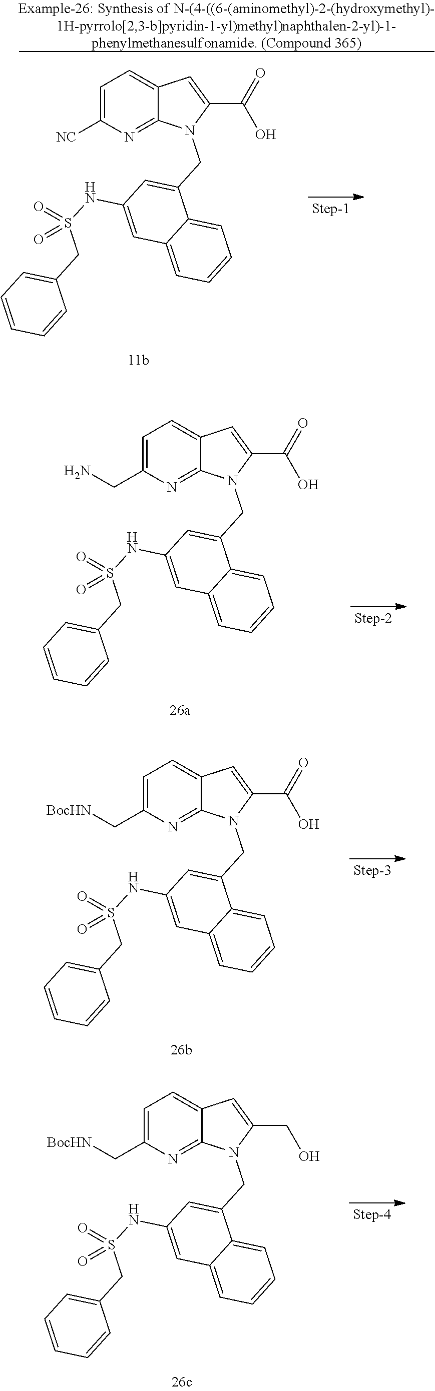 [SMILES: N#Cc1ccc2cc(C(=O)O)n(Cc3cc(NS(=O)(=O)Cc4ccccc4)cc4ccccc34)c2n1 ; NCc1ccc2cc(C(=O)O)n(Cc3cc(NS(=O)(=O)Cc4ccccc4)cc4ccccc34)c2n1 ; CC(C)(C)OC(=O)NCc1ccc2cc(C(=O)O)n(Cc3cc(NS(=O)(=O)Cc4ccccc4)cc4ccccc34)c2n1 ; CC(C)(C)OC(=O)NCc1ccc2cc(CO)n(Cc3cc(NS(=O)(=O)Cc4ccccc4)cc4ccccc34)c2n1]

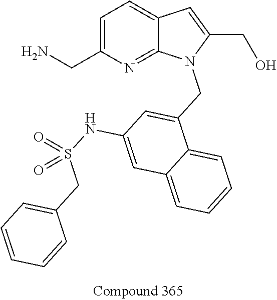 [SMILES: NCc1ccc2cc(CO)n(Cc3cc(NS(=O)(=O)Cc4ccccc4)cc4ccccc34)c2n1]

### Step-1: Synthesis of 6-(aminomethyl)-1-(3-((phenylmethyl)sulfonamido)naphthalen-1-yl)methyl)-1H-pyrrolo[2,3-b]pyridine-2-carboxylic acid

The experimental procedure was the same as described in Step-4 of Example-1 with appropriate variations in coupling methods, reactants, quantities of reagents, and solvents to obtain the compound 26a (1.9 g). LCMS: 501.30 [M+H]⁺.

### Step-2: Synthesis of 6-(((tert-butoxycarbonyl)amino)methyl)-1-((3-((phenylmethyl)sulfonamido)naphthalen-1-yl)methyl)-1H-pyrrolo[2,3-b]pyridine-2-carboxylic acid

The experimental procedure was the same as described in Step-4 of Example-23 with appropriate variations in coupling methods, reactants, quantities of reagents, and solvents to obtain the compound 26b (1.85 g). LCMS: 601.0 [M+H]⁺.

### Step-3: Synthesis of tert-butyl ((2-(hydroxymethyl)-1-((3-((phenylmethyl)sulfonamido)naphthalen-1-yl)methyl)-1H-pyrrolo[2,3-b]pyridin-6-yl)methyl)carbamate

The experimental procedure was the same as described in Step-5 of Example-23 with appropriate variations in coupling methods, reactants, quantities of reagents, and solvents to obtain the compound 26c (0.189 g). LCMS: 587.40 [M+H]⁺.

### Step-4: Synthesis of N-(4-((6-(aminomethyl)-2-(hydroxymethyl)-1H-pyrrolo[2,3-b]pyridin-1-yl)methyl)naphthalen-2-yl)-1-phenylmethanesulfonamide

The experimental procedure was the same as described in Step-5 of Example-1 with appropriate variations in coupling methods, reactants, quantities of reagents, and solvents to obtain the compound (Compound 265) (0.045 g). LC-MS: 487.2 [M+H]⁺; ¹HNMR (CD₃OD, 400 MHz): δ 4.22-4.28 (d, 4H), 4.67-4.68 (d, 2H), 6.24 (s, 2H), 6.41 (s, 1H), 6.66-6.70 (m, 1H), 7.08-7.25 (m, 6H), 7.48-7.57 (m, 3H), 7.79-7.82 (m, 1H), 8.08-8.10 (m, 1H), 8.20-8.24 (m, 1H).

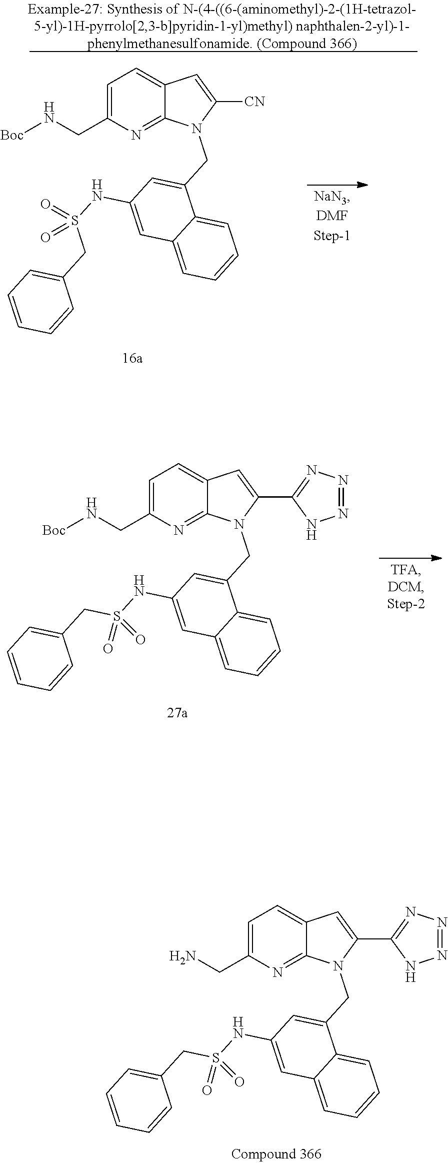 [SMILES: CC(C)(C)OC(=O)NCc1ccc2cc(C#N)n(Cc3cc(NS(=O)(=O)Cc4ccccc4)cc4ccccc34)c2n1 ; CC(C)(C)OC(=O)NCc1ccc2cc(-c3nnn[nH]3)n(Cc3cc(NS(=O)(=O)Cc4ccccc4)cc4ccccc34)c2n1 ; NCc1ccc2cc(-c3nnn[nH]3)n(Cc3cc(NS(=O)(=O)Cc4ccccc4)cc4ccccc34)c2n1]

### Step-1: Synthesis of tert-butyl ((1-((3-((phenylmethyl)sulfonamido)naphthalen-1-yl)methyl)-2-(1H-tetrazol-5-yl)-1H-pyrrolo[2,3-b]pyridin-6-yl)methyl)carbamate

A mixture of tert-butyl ((2-cyano-1-((3-((phenylmethyl)sulfonamido)naphthalen-1-yl)methyl)-1H-pyrrolo[2,3-b]pyridin-6-yl)methyl)carbamate (0.14 g, 0.24 mmol), NH₄C₁ (0.076 g, 1.49 mmol) and NaN3 (0.047 g, 0.72 mmol) in DMF (3 mL) was heated to 110° C. for 15 h. The reaction mixture was concentrated and washed with diethyl ether to yield the title compound which was used as such in the next step without any purification to obtain the compound 27a (0.1 g). LC-MS: 625.30 [M+H]⁺.

### Step-2: Synthesis of N-(4-((6-(aminomethyl)-2-(1H-tetrazol-5-yl)-1H-pyrrolo[2,3-b]pyridin-1-yl)methyl)naphthalen-2-yl)-1-phenylmethanesulfonamide

The experimental procedure was the same as described in Step-5 of Example-1 with appropriate variations in coupling methods, reactants, quantities of reagents, and solvents to obtain the title compound (Compound 366) (0.04 g). LC-MS: 525.30 [M+H]⁺; ¹HNMR (CD₃OD, 400 MHz): δ 4.09 (s, 2H), 4.31 (s, 2H), 6.28 (s, 1H), 6.70 (s, 2H), 6.91-6.93 (d, 2H), 7.07-7.33 (m, 4H), 7.36-7.39 (m, 2H), 7.51-7.56 (m, 2H), 7.72-7.75 (m, 1H), 8.19-8.23 (m, 2H).

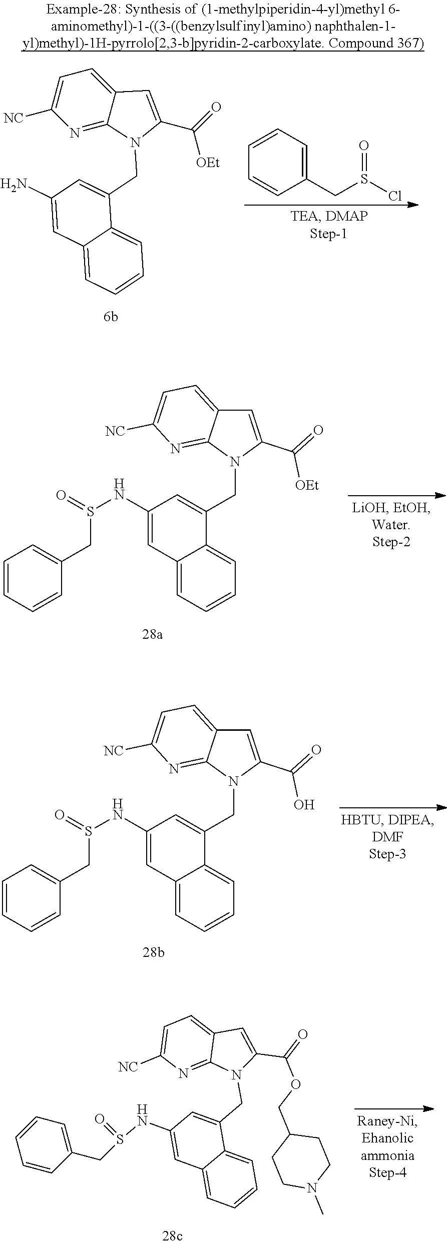 [SMILES: CCOC(=O)c1cc2ccc(C#N)nc2n1Cc1cc(N)cc2ccccc12 ; O=S(Cl)Cc1ccccc1 ; CCOC(=O)c1cc2ccc(C#N)nc2n1Cc1cc(NS(=O)Cc2ccccc2)cc2ccccc12 ; N#Cc1ccc2cc(C(=O)O)n(Cc3cc(NS(=O)Cc4ccccc4)cc4ccccc34)c2n1 ; CN1CCC(COC(=O)c2cc3ccc(C#N)nc3n2Cc2cc(NS(=O)Cc3ccccc3)cc3ccccc23)CC1]

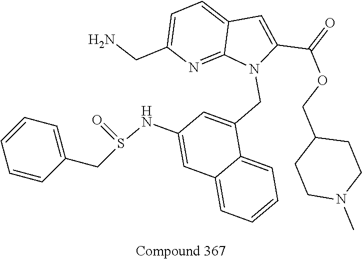 [SMILES: CN1CCC(COC(=O)c2cc3ccc(CN)nc3n2Cc2cc(NS(=O)Cc3ccccc3)cc3ccccc23)CC1]

### Step-1: Synthesis of ethyl 1-((3-(benzylsulfinyl)amino)naphthalen-1-yl)methyl)-6-cyano-1H-pyrrolo[2,3-b]pyridine-2-carboxylate

A solution of ethyl 1-((3-aminonaphthalen-1-yl)methyl)-6-cyano-1H-pyrrolo[2,3-b]pyridine-2-carboxylate (0.2 g, 0.54 mmol) in DCM (2 mL) was cooled to 0° C. and to the solution was added Et3N (0.164 g, 1.62 mmol), DMAP (0.033 g, 0.27 mmol) and phenylmethanesulfinic chloride (0.189 g, 1.08 mmol) and stirred at RT for 12 h. The reaction mixture was diluted with DCM, washed with brine solution, and then washed with saturated NaHCO₃ solution, dried over Na₂SO₄, and concentrated. The crude compound was purified by Combi flash chromatography using 50% ethyl acetate in hexane as eluent. This afforded the title compound 28a (0.045 g, 16.3%). LC-MS: 508.90 [M+H]⁺.

### Step-2: Synthesis of 1-(3-(benzylsulfinyl)amino)naphthalen-1-yl)methyl)-6-cyano-1H-pyrrolo[2,3-b]pyridine-2-carboxylic acid

The experimental procedure was the same as described in Step-3 of Example-1 with appropriate variations in coupling methods, reactants, quantities of reagents, and solvents to obtain the compound 28b (0.215 g). LC-MS: 481.30 [M+H]⁺.

### Step-3: Synthesis of (1-methylpiperidin-4-yl)methyl 1-(3-((benzylsulfinyl)amino)naphthalen-1-yl)methyl)-6-cyano-1H-pyrrolo[2,3-b]pyridine-2-carboxylate

The experimental procedure was the same as described in Step-6 of Example-1 with appropriate variations in coupling methods, reactants, quantities of reagents, and solvents to obtain the compound 28c (0.2 g). LC-MS: 592.40 [M+H]⁺.

### Step-4: Synthesis of (1-methylpiperidin-4-yl)methyl 6-(aminomethyl)-1-(3-((benzylsulfinyl)amino)naphthalen-1-yl)methyl)-1H-pyrrolo[2,3-b]pyridine-2-carboxylate

The experimental procedure was the same as described in Step-8 of Example-1 with appropriate variations in coupling methods, reactants, quantities of reagents, and solvents to obtain the compound (Compound 367) (0.019 g, 9.4%). (Prep HPLC method: Column-Waters, Xbridge, C18 (250 mm×21.1 mm), flow rate-16 mL/min, mobile phase-A was 5M Aqueous NH4OAc, B was acetonitrile, Gradient program-20% B at 0 minute, 30% B at 2 minutes, 60% B at 10 minutes). LC-MS: 596.40 [M+H]⁺; ¹HNMR (CD₃OD, 400 MHz): δ 1.12-1.15 (m, 2H), 1.29-1.39 (m, 4H), 1.73-1.80 (m, 1H), 2.23 (s, 3H), 2.70-2.75 (m, 2H), 4.00-4.02 (d, 2H), 4.19 (s, 2H), 4.55 (s, 2H), 6.00 (s, 1H), 6.46-6.47 (d, 2H), 7.23-7.30 (m, 6H), 7.51-7.55 (m, 3H), 7.76-7.78 (m, 1H), 8.17-8.19 (m, 2H), 8.24-8.26 (m, 1H).

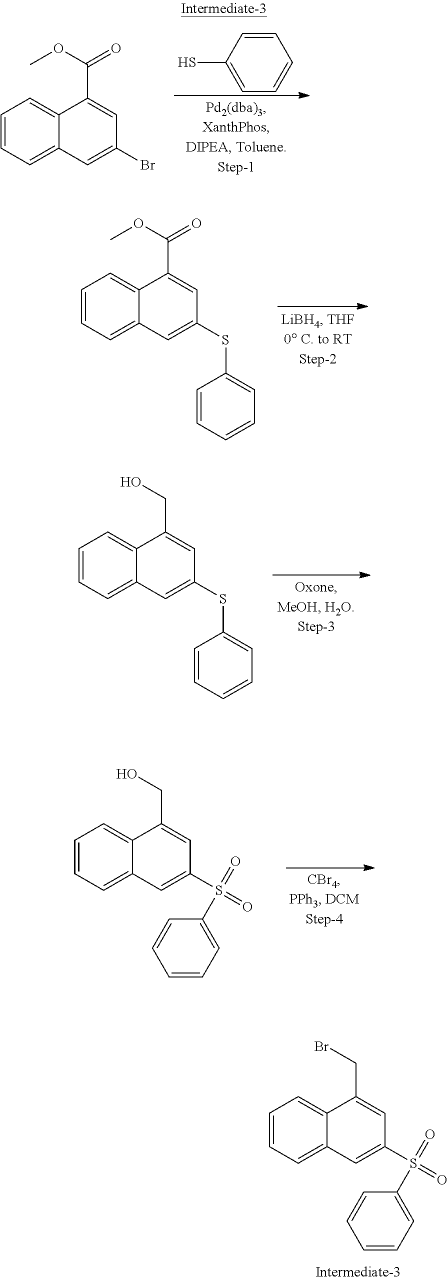 [SMILES: COC(=O)c1cc(Br)cc2ccccc12 ; Sc1ccccc1 ; COC(=O)c1cc(Sc2ccccc2)cc2ccccc12 ; OCc1cc(Sc2ccccc2)cc2ccccc12 ; O=S(=O)(c1ccccc1)c1cc(CO)c2ccccc2c1 ; O=S(=O)(c1ccccc1)c1cc(CBr)c2ccccc2c1]

### Step-1: Synthesis of methyl 3-(phenylthio)-1-naphthoate

To a stirred solution of methyl 3-bromo-1-naphthoate (5 g, 19.01 mmol) in Toluene (60 mL) was added benzenethiol (2.52 g, 22.81 mmol). The reaction mixture was degassed for 15 minutes. To this DIPEA (7.2 g, 5.76 mmol) was added and degassed for 10 minutes. Then Pd2(dba)3 (0.34 g, 0.381 mmol) and Xanthphos (0.54 g, 0.951 mmol) were added and the reaction mixture was heated to 100° C. for 8 hours. After completion of the reaction mixture was diluted with EtOAc and filtered. The filtrate was diluted with water and layers were separated. The organic layer was dried over anhydrous Na₂SO₄ and concentrated under reduced pressure. The crude was purified by Combi flash chromatography using 1% ethyl acetate in hexane as eluent to afford the pure title compound (4.6 g, crude). LC-MS: 294.4 [M+H]⁺.

### Step-2: Synthesis of (3-(phenylthio)naphthalen-1-yl)methanol

To a stirred solution of methyl 3-(phenylthio)-1-naphthoate (1 g, 3.47 mmol) in THF (10 mL) was added LiBH4 (3.7 g, 10.79 mmol). The reaction mixture was then stirred for 4 h at RT. Then the reaction mixture was quenched with aqueousNH4Cl solution at 0° C. and diluted with EtOAc. The filtrate was diluted with water and layers were separated. The organic layer was dried over anhydrous Na₂SO₄ and concentrated under reduced pressure to get the crude product (3.6 g, crude) which was used for the next step without further purification. LC-MS: 267.3 [M+H]⁺.

### Step-3: Synthesis of (3-(phenylsulfonyl)naphthalen-1-yl)methanol

To a stirred solution of (3-(phenylthio)naphthalen-1-yl)methanol (0.8 g, 3.00 mmol) in MeOH (30 mL) was added the solution of Oxone (2.2 g, 15.03 mmol) in H2O (5 mL). The reaction mixture was stirred at RT for 4 h. Then the reaction mixture was concentrated and to the concentrates was added ice-cold water. Solid was obtained and filtered to yield the title compound (0.5 g, crude).

### Step-4: Synthesis of 1-(bromomethyl)-3-(phenylsulfonyl)naphthalene

To a stirred solution of (3-(phenylsulfonyl)naphthalen-1-yl)methanol (0.5 g, 1.67 mmol) in DCM (20 mL) was added PPh₃ (0.75 g, 3.36 mmol) and CBr4 (0.12 g, 3.36 mmol). The reaction mixture was stirred at RT for 1 h. Then the reaction mixture was concentrated to afford the crude product and was purified by Combi flash chromatography using 10% MeOH in DCM as eluent to afford the pure title compound (Intermediate-3) (0.35 g, 74%) LC-MS: 361.0 [M+H]⁺.

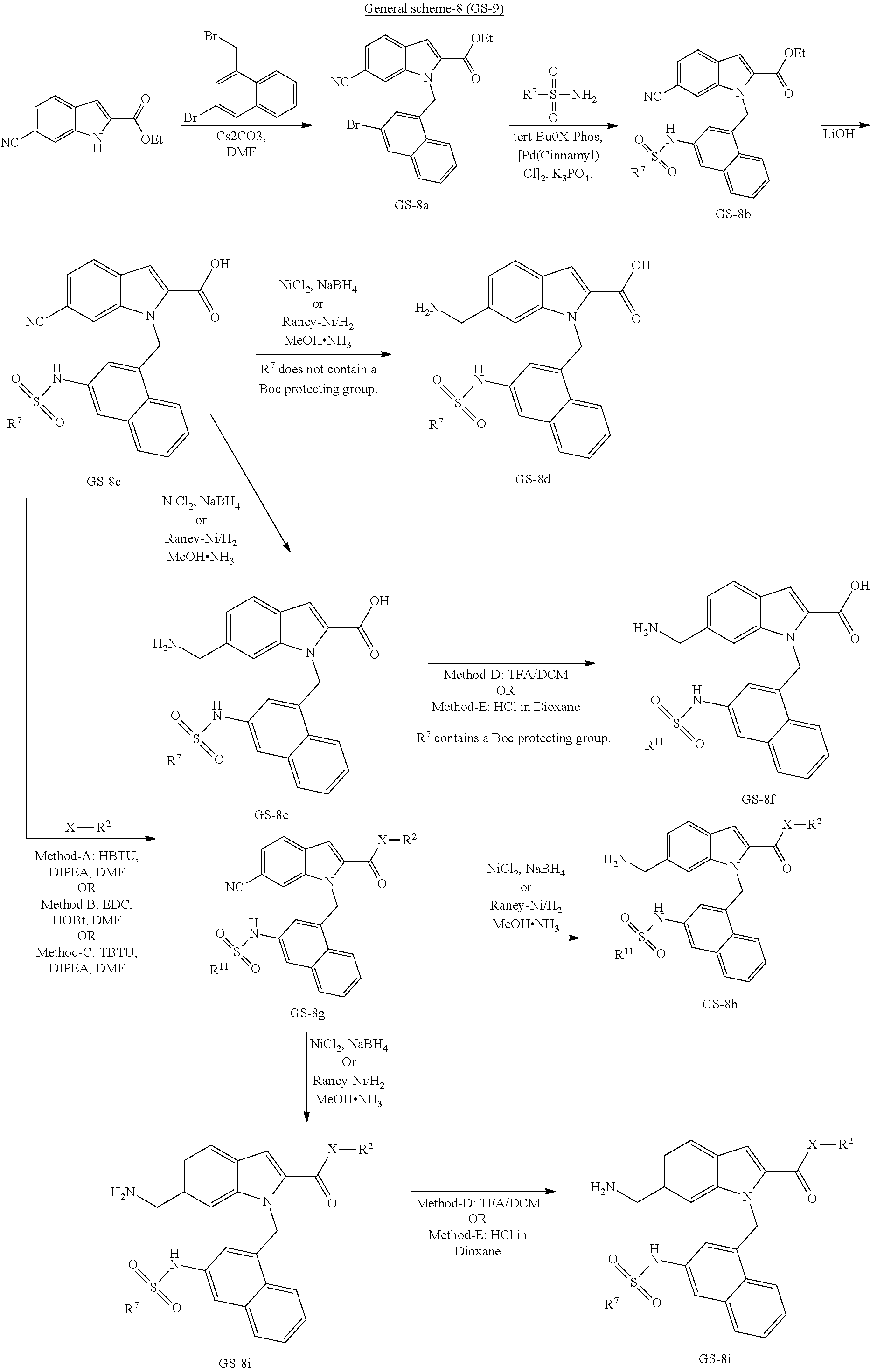

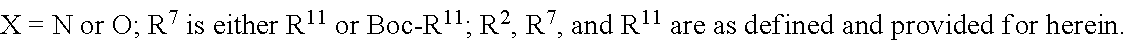

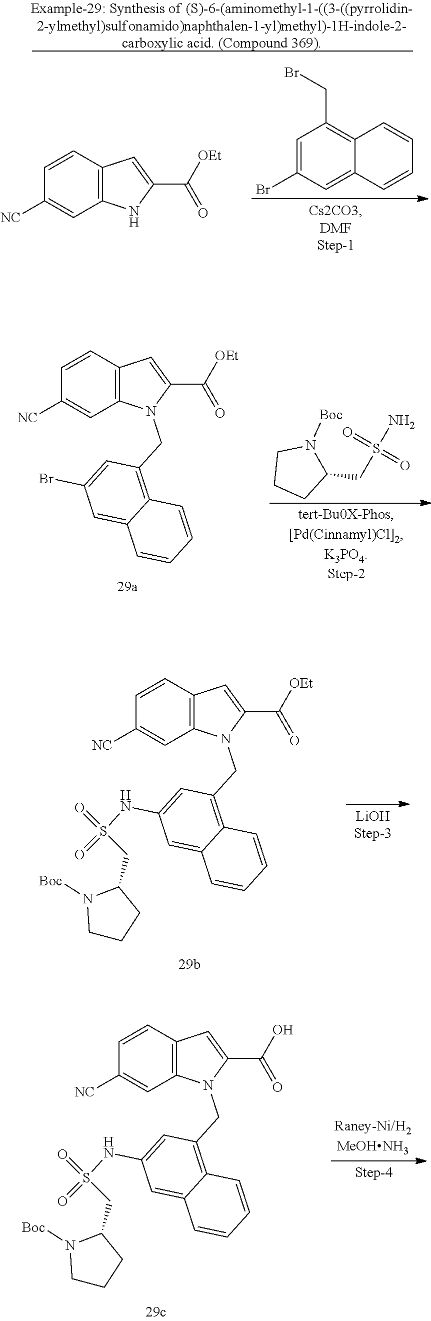 [SMILES: CCOC(=O)c1cc2ccc(C#N)cc2[nH]1 ; BrCc1cc(Br)cc2ccccc12 ; CCOC(=O)c1cc2ccc(C#N)cc2n1Cc1cc(Br)cc2ccccc12 ; CC(C)(C)OC(=O)N1CCC[C@H]1CS(N)(=O)=O ; CCOC(=O)c1cc2ccc(C#N)cc2n1Cc1cc(NS(=O)(=O)C[C@@H]2CCCN2C(=O)OC(C)(C)C)cc2ccccc12 ; CC(C)(C)OC(=O)N1CCC[C@H]1CS(=O)(=O)Nc1cc(Cn2c(C(=O)O)cc3ccc(C#N)cc32)c2ccccc2c1]

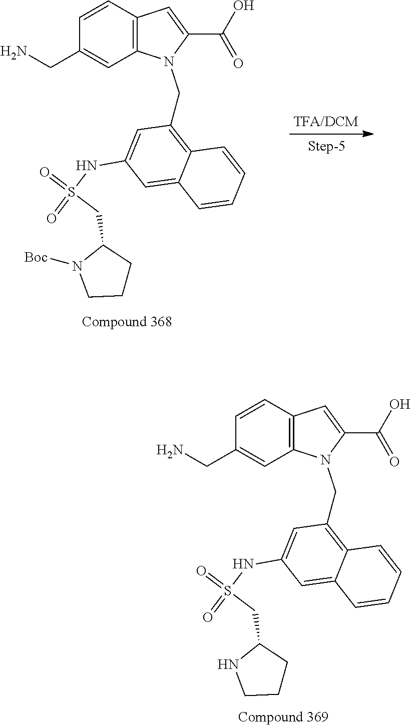 [SMILES: CC(C)(C)OC(=O)N1CCC[C@H]1CS(=O)(=O)Nc1cc(Cn2c(C(=O)O)cc3ccc(CN)cc32)c2ccccc2c1 ; NCc1ccc2cc(C(=O)O)n(Cc3cc(NS(=O)(=O)C[C@@H]4CCCN4)cc4ccccc34)c2c1]

### Step-1: Synthesis of ethyl 1-((3-bromonaphthalen-1-yl)methyl)-6-cyano-1H-indole-2-carboxylate

To a solution of ethyl 6-cyano-1H-indole-2-carboxylate (6.46 g, 30 mmol) in DMF (100 mL) was added Cs₂CO₃ (43.7 g, 134 mmol) and 3-bromo-1-(bromomethyl)naphthalene (10 g, 33 mmol) respectively. After addition, the mixture was stirred at RT for 15 h. The reaction mixture was poured into ice-cold water, washed with hexane followed by 5% ethyl acetate in hexane, the precipitate formed was filtered and dried to yield the title compound 29a (13.2 g.) LCMS: 430.9 [M−H]⁻.

### Step-2: Synthesis of ethyl(S)-1-(3-(((1-(tert-butoxycarbonyl)pyrrolidin-2-yl)methyl)sulfonamido)naphthalen-1-yl)methyl)-6-cyano-1H-indole-2-carboxylate

A solution of ethyl 1-((3-bromonaphthalen-1-yl)methyl)-6-cyano-1H-indole-2-carboxylate (1.25 g, 2.89 mmol), tert-butyl(S)-2-(sulfamoylmethyl)pyrrolidine-1-carboxylate (0.91 g, 3.47 mmol) in 1,4-Dioxane (30 ml) was purged with nitrogen, added K₃PO₄ (1.83 g, 8.67 mmol) followed by Palladium Cinnamyl Chloride dimer (0.14 g, 0.28 mmol) and tert butyl-XPhos (0.24 g, 0.57 mmol). After addition, the mixture was stirred at 90° C. for 15 h. The reaction mixture was then filtered through Celite, washed with ethyl acetate and the filtrate was concentrated to get crude. The crude compound was purified by column chromatography (liquid packing). By eluting with of 50% EtOAc in Hexane. This afforded the pure title compound 29b (0.94 g). LC-MS: 615.3 [M−H]⁻.

### Step-3: Synthesis of(S)-1-((3-(((1-(tert-butoxycarbonyl)pyrrolidin-2-yl)methyl)sulfonamido)naphthalen-1-yl)methyl)-6-cyano-1H-indole-2-carboxylic acid

A solution of ethyl(S)-1-((3-(((1-(tert-butoxycarbonyl)pyrrolidin-2-yl)methyl)sulfonamido)naphthalen-1-yl)methyl)-6-cyano-1H-indole-2-carboxylate (0.94 g, 1.52 mmol) in THF (13 ml) and EtOH (3 ml) was cooled to 0° C. This was added LiOH·H2O (0.192 g, 4.57 mmol) in water (5 mL). Then the reaction mixture was warmed to RT and stirred for 4 h. The reaction mixture was concentrated, and the residue was dissolved in cold water, acidified with 1N HCl (pH˜4.0-5.0). The product precipitated was filtered and dried under vacuum to yield the title compound 29c (0.86 g, 95%). LC-MS: 587.30 [M−H]⁻.

### Step-4: Synthesis of(S)-6-(aminomethyl)-1-((3-(((1-(tert-butoxycarbonyl)pyrrolidin-2-yl)methyl)sulfonamido)naphthalen-1-yl)methyl)-1H-indole-2-carboxylic acid DISC-0001877

To a solution of(S)-1-((3-(((1-(tert-butoxycarbonyl)pyrrolidin-2-yl)methyl)sulfonamido)naphthalen-1-yl)methyl)-6-cyano-1H-indole-2-carboxylic acid (0.07 g, 0.119 mmol) in MeOH (2 ml) was added methanolic ammonia (5 mL) and Raney nickel (0.1 g) at RT and the reaction mixture was stirred for 2 h under positive pressure of hydrogen using a bladder. The reaction mixture was filtered through Celite, and the filtrate was concentrated to yield the title compound (Compound 368) (0.07 g). LC-MS: 593.1 [M+H]⁺; ¹HNMR (CD₃OD, 400 MHz): δ 1.18-1.39 (m, 9H), 1.74 (bs, 2H), 1.91-1.96 (m, 3H), 2.88-2.94 (t, 1H), 3.32 (s, 3H), 4.03-4.11 (m, 3H), 6.19 (s, 1H), 6.40 (s, 2H), 7.21-7.23 (d, 1H), 7.41-7.55 (m, 4H), 7.82-7.84 (d, 2H), 8.17 (s, 1H).

### Step-5: Synthesis of(S)-6-(aminomethyl)-1-((3-((pyrrolidin-2-ylmethyl)sulfonamido)naphthalen-1-yl)methyl)-1H-indole-2-carboxylic acid. (Compound 369)

A solution of(S)-6-(aminomethyl)-1-((3-(((1-(tert-butoxycarbonyl)pyrrolidin-2-yl)methyl)sulfonamido)naphthalen-1-yl)methyl)-1H-indole-2-carboxylic acid (0.06 g, 0.0.101 mmol) in Dioxane (3 mL) was cooled to 0° C. and to the solution was added HCl in Dioxane (0.7 mL). The reaction mixture was stirred at RT for 16 h and concentrated to get the crude mass. This was purified by preparative-HPLC to afford pure title compound (Compound 369) (0.022 g). (Preparative HPLC method: Column-Luna Omega-C18 (250 mm×21.2 mm), 5.0μ; Eluent-A 0.05% TFA in water. B—is Acetonitrile, gradient-10% B at 0-minute, 20% B at 2 minutes, 45% at 10 minutes). LC-MS: 493.1[M+H]⁺; ¹HNMR (CD₃OD, 400 MHz): δ 1.60-1.66 (m, 1H), 1.92-2.04 (m, 2H), 2.16-2.22 (m, 1H), 3.26-3.55 (d, 2H), 3.34-3.43 (m, 2H), 3.80-3.81 (m, 1H), 4.12 (s, 2H), 6.19 (s, 1H), 6.34-6.44 (q, 2H), 7.21-7.24 (d, 1H), 7.44-7.46 (d, 2H), 7.55-7.60 (m, 3H), 7.82-7.86 (m, 2H), 8.19-8.21 (d, 1H).

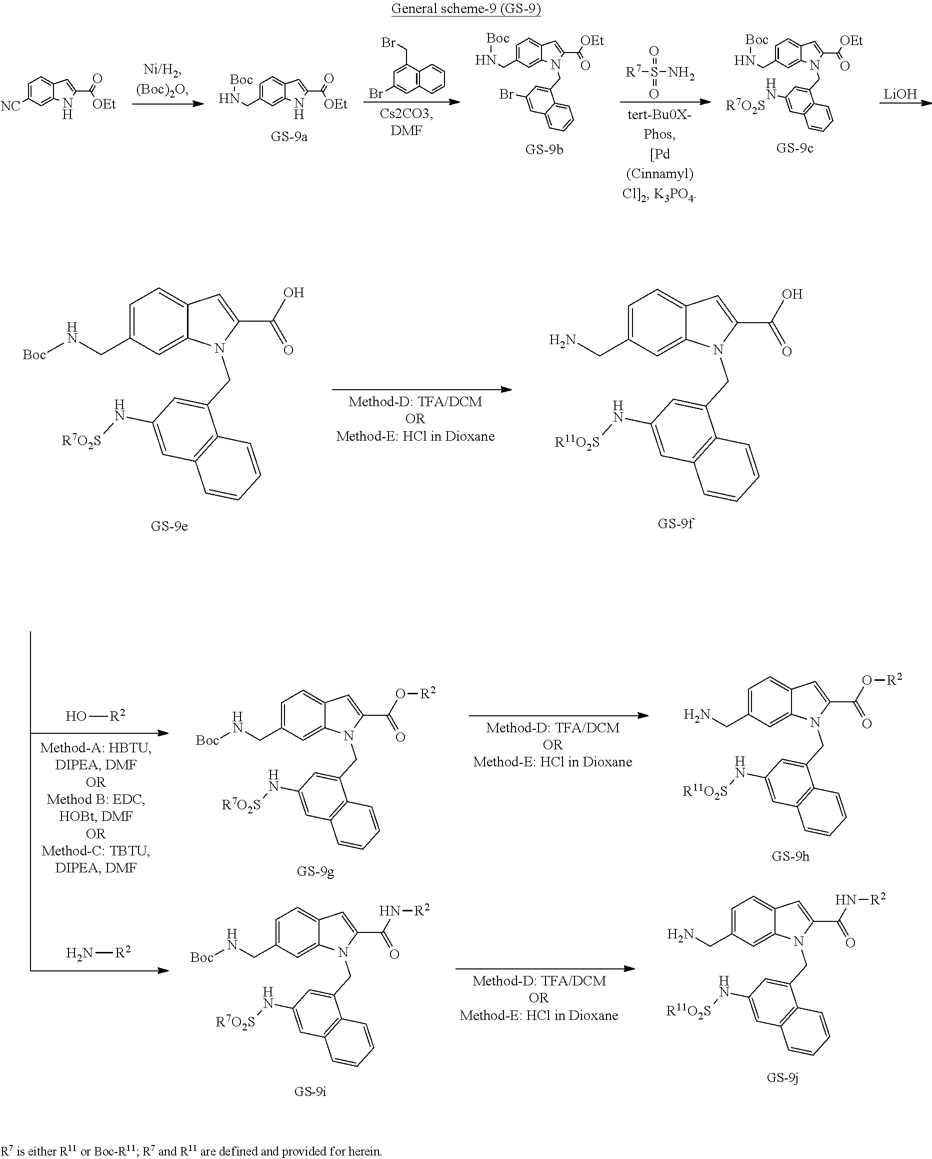

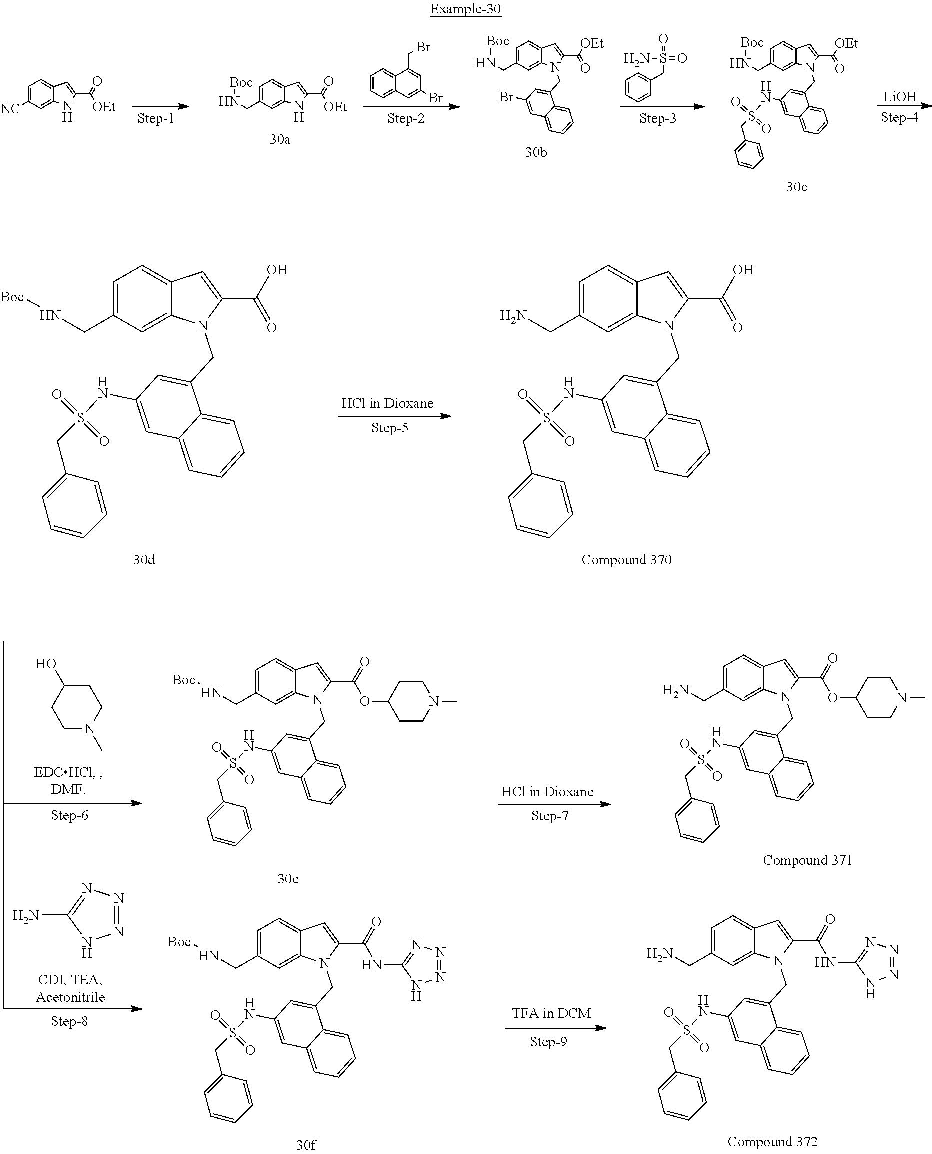

### Step-1: Synthesis of ethyl 6-(((tert-butoxycarbonyl)amino)methyl)-1H-indole-2-carboxylate

A stirred solution of ethyl 6-cyano-1H-indole-2-carboxylate (1 g, 4.6 mmol) in methanol (50 mL) was cooled to 0° C. and to the solution was added Boc anhydride (2.03 g, 9.3 mmol) followed by NiCl₂·6H₂O (0.11 g, 0.46 mmol). NaBH₄ (1.2 g, 32.6 mmol) was added in portions by maintaining the same temperature. The reaction mixture was stirred at RT for 2 h. The reaction mixture was concentrated, the residue obtained was dissolved in ethyl acetate, washed with sat. NaHCO₃ solution followed by brine solution, dried over Na₂SO₄ and concentrated to yield the title compound 30a (1.3 g, 87.4%). LC-MS: 317.3 [M−H]⁻.

### Step-2: Synthesis of ethyl 1-((3-bromonaphthalen-1-yl)methyl)-6-(((tert-butoxycarbonyl)amino)methyl)-1H-indole-2-carboxylate

To a solution of ethyl 6-(((tert-butoxycarbonyl)amino)methyl)-1H-indole-2-carboxy late (1.3 g, 4.08 mmol) in DMF (15 mL) was added Cs₂CO₃ (3.99 g, 12.2 mmol) followed by 3-bromo-1-(bromomethyl)naphthalene (1.22 g, 4.08 mmol). The reaction mixture was stirred at RT for 12 h. Then the reaction mixture was poured into ice-cold water, the precipitate formed was filtered and dried. This was purified by Combi flash chromatography using DCM as eluent to afford the pure title compound 30b (1.3 g, 59.2%). LC-MS: 538.40 [M+2]⁺.

### Step-3: Synthesis of ethyl 6-(((tert-butoxycarbonyl)amino methyl)-1-(3-((phenylmethyl)sulfonamido)naphthalen-1-yl)methyl)-1H-indole-2-carboxylate

A mixture of 1-((3-bromonaphthalen-1-yl)methyl)-6-(((tert-butoxycarbonyl)amino)methyl)-1H-indole-2-carboxylate (1.3 g, 2.41 mmol), phenylmethanesulfonamide (0.41 g, 2.41 mmol) in dioxane (40 mL) was added K₃PO₄ (1.28 g, 6.04 mmol) and purged with N₂. The reaction mixture was then added tert-butyl-Xphos (0.077 g, 0.18 mmol), palladium(π-cinnamyl) chloride dimer (0.047 g, 0.09 mmol) and heated to 90° C. for 4 h. The reaction mixture was filtered through Celite. and the collected filtrate was concentrated to get crude mass. This was purified by Combi-flash column chromatography using DCM as eluent to afford the pure title compound 30c (1.1 g, 72.44%). LC-MS: 629.40 [M+H]⁺.

### Step-4: Synthesis of 6-(((tert-butoxycarbonyl)amino methyl)-1-(3-((phenylmethyl)sulfonamido)naphthalen-1-yl)methyl)-1H-indole-2-carboxylic acid

A mixture of ethyl 6-(((tert-butoxycarbonyl)amino)methyl)-1-((3-((phenylmethyl)sulfonamido)naphthalen-1-yl)methyl)-1H-indole-2-carboxylate (1.1 g, 1.75 mmol), ethanol (5 mL) and THF (5 mL) was added LiOH monohydrate (0.36 g, 8.78 mmol) solution in water (5 mL) at 0° C. and stirred at RT for 1 h. The reaction mixture was concentrated, and the residue was dissolved in water. The aqueous portion was acidified using 1N HCl extracted with ethyl acetate, dried over Na₂SO₄, concentrated to yield the title compound 30d (1 g, 95.18%). LC-MS: 601.3 [M+H]⁺.

### Step-5: Synthesis of 6-(aminomethyl)-1-((3-((phenylmethyl)sulfonamido)naphthalen-1-yl)methyl)-1H-indole-2-carboxylic acid. (Compound 370)

A stirred solution of 6-(((tert-butoxy carbonyl)amino)methyl)-1-((3-((thiophen-3-ylmethyl)sulfonamido)naphthalen-1-yl)methyl)-1H-pyrrolo[2,3-b]pyridine-2-carboxylic acid (0.5 g, 0.82 mmol) in DMF (8 mL) was cooled to 0° C. added HBTU (0.62 g, 1.64 mmol), N,N-DIPEA (0.42 g, 3.29 mmol) and stirred for 15 minutes at the same temperature. Then the reaction mixture was added (1-ethylpiperidin-4-yl)methanol (0.42 g, 3.29 mmol) and stirred for 15 h at RT. The reaction mixture was quenched by adding water, precipitated formed was filtered, washed with water and dried. The crude compound was purified by Combi-flash column chromatography using 0-3.5% methanol in DCM as eluent to afford the pure title compound (Compound 370) (0.48 g, 81.1%). LC-MS: 500 [M+H]⁺; ¹HNMR (CD₃OD, 400 MHz): δ 4.12 (s, 2H), 4.18 (s, 2H), 6.22-6.23 (d, 1H), 6.45 (s, 2H), 7.05-7.08 (dd, 2H), 7.21-7.27 (m, 4H), 7.46 (s, 1H), 7.51 (s, 1H), 7.56-7.60 (m, 3H), 7.81-7.88 (m, 2H), 8.21-8.24 (t, 1H).

### Step-6: Synthesis of 1-methylpiperidin-4-yl 6-(((tert-butoxycarbonyl)amino methyl)-1-(3-((phenylmethyl)sulfonamido)naphthalen-1-yl)methyl)-1H-indole-2-carboxylate

To a solution of 6-(((tert-butoxycarbonyl)amino)methyl)-1-((3-((phenylmethyl)sulfonamido)naphthalen-1-yl)methyl)-1H-indole-2-carboxylic acid (0.225 g, 0.375 mmol) in DMF (2 mL) was added EDC·HCl (0.216 g, 1.12 mmol), DMAP (0.068 g, 0.56 mmol) and stirred for 10 minutes. Then 1-methylpiperidin-4-ol (0.21 g, 1.67 mmol) was added and the mixture was stirred at RT for 18 h. The reaction mixture was added into ice-cold water, precipitate formed was filtered and washed with water and dried. This afforded title compound 30e (0.235 g, 89.97%). LC-MS: 697.4 [M+H]⁺.

### Step-7: Synthesis of 1-methylpiperidin-4-yl 6-(aminomethyl)-1-((3-((phenylmethyl)sulfonamido)naphthalen-1-yl)methyl)-1H-indole-2-carboxylate. (Compound 371)

To a stirred solution of tert-butyl 1-methylpiperidin-4-yl 6-(((tert-butoxycarbonyl)amino)methyl)-1-((3-((phenylmethyl)sulfonamido)naphthalen-1-yl)methyl)-1H-indole-2-carboxylate (0.26 g, 0.373 mmol) in DCM (5 mL) was added TFA (0.5 mL) and stirred for 2 h at RT. The reaction mixture was then concentrated, added diethyl ether and filtered to get crude compound (Compound 371) upon purified by preparative HPLC. (Preparative HPLC Method: Column: LUNA OMEGA PS (250×21.2 mm) 5μ; Mobile Phase A: 0.05% TFA in Water, B: Acetonitrile; Flow rate: 20 mL/min; Gradient: % B—20 at 0 minute, 30 at 2 minutes, 70 at 6 minutes). LC-MS: 596.40 [M+H]⁺; ¹HNMR (CD₃OD, 400 MHz): δ 1.85-2.25 (m, 4H), 2.62 (s, 3H), 2.87-2.95 (m, 2H), 3.10-3.25 (m, 2H), 3.45-3.55 (m, 1H), 3.98 (s, 2H), 4.00 (s, 2H), 6.31 (s, 1H), 6.61 (s, 2H), 7.04-7.06 (m, 2H), 7.19-7.24 (m, 4H), 7.41-7.47 (m, 1H), 7.54-7.65 (m, 3H), 7.78 (s, 1H), 781-7.93 (m, 2H), 8.22-8.27 (m, 1H).

### Step-8: Synthesis of tert-butyl (2-(1H-tetrazol-5-yl) carbamoyl)-1-(3-((phenylmethyl)sulfonamido)naphthalen-1-yl)methyl)-1H-indol-6-yl)methyl)carbamate

To a solution of 1H-tetrazol-5-amine (0.051 g, 0.6 mmol), Et₃N (0.126 g, 1.25 mmol) in acetonitrile (15 mL) was added CDI (0.1 g, 0.6 mmol) and stirred for 1 h at 80° C. Then 6-(((tert-butoxycarbonyl)amino)methyl)-1-((3-((phenylmethyl)sulfonamido)naphthalen-1-yl)methyl)-1H-indole-2-carboxylic acid (0.3 g, 0.5 mmol) was added and the mixture was refluxed for 12 h. The reaction mixture was cooled to RT, added into ice-cold water, precipitate formed was filtered and washed with water and dried to get title compound 30f (0.15 g). LC-MS: 667.3 [M+H]⁺.

### Step-9: Synthesis of 6-(aminomethyl)-1-((3-((phenylmethyl)sulfonamido)naphthalen-1-yl)methyl)-N-(1H-tetrazol-5-yl)-1H-indole-2-carboxamide. (Compound 372)

To a stirred solution of tert-butyl ((2-((1H-tetrazol-5-yl) carbamoyl)-1-((3-((phenylmethyl)sulfonamido)naphthalen-1-yl)methyl)-1H-indol-6-yl)methyl)carbamate (0.15 g, 0.22 mmol) in DCM (5 mL) was added TFA (0.256 g, 2.24 mmol) and stirred for 2 h at RT. The reaction mixture was then concentrated. The residue was purified by preparative HPLC to get the pure title compound (Compound 372) (0.12 g). (Prep HPLC method: Column-GEMINI NX (150 mm×21.2 mm), flow rate-20 mL/min, mobile phase-A was 0.1% TFA in water, B was acetonitrile, Gradient program-10% B at 0-minute, 20% B at 2 minutes, 30% B at 8 minutes). LC-MS: 567.1 [M+H]⁺; ¹HNMR (CD₃OD, 300 MHz): δ 4.12 (s, 2H), 4.14 (s, 2H), 6.35 (s, 1H), 6.48 (s, 2H), 6.98-7.03 (m, 2H), 7.06-7.16 (m, 3H), 7.28-7.31 (d, 1H), 7.44 (s, 1H), 7.53-7.59 (m, 3H), 7.79-7.83 (m, 2H), 7.91-7.94 (d, 1H), 8.21-8.24 (m, 1H).

The following compounds listed in Table 6 were prepared according to GS-8 or GS-9 by following the similar procedures as described above for Example-29 or Example-30 using appropriate reagents with suitable modifications known to the one skilled in the art.

TABLE 6

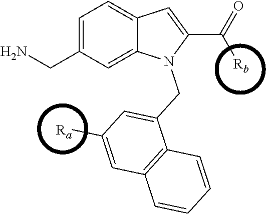

| Cmpd # | Rₐ | R_{b} | M | Spectral data |
|---|---|---|---|---|
| 373 | 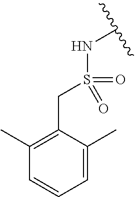 [SMILES: *NS(=O)(=O)Cc1c(C)cccc1C] | 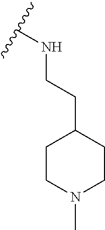 [SMILES: *NCCC1CCN(C)CC1] | GS-2 | LC-MS: 650.3 [M − H]⁻; ¹HNMR (CD₃OD, 400 MHz): δ 1.22-1.29 (m, 2H), 1.42-1.45 (q, 2H), 1.84-1.88 (d, 2H), 2.15 (s, 6H), 2.43-2.49 (t, 2H), 2.65 (s, 3H) 3.24-3.27 (m, 3H), 3.33-3.36 (m, 2H), 4.13 (s, 2H), 4.36 (s, 2H), 6.32 (s, 1H), 6.43 (s, 2H), 6.93-6.95 (d, 2H), 7.03-7.07 (t, 1H), 7.24-7.28 (m, 2H), 7.37 (b, 1H), 7.516 (s, 1H), 7.58-7.59 (t, 2H), 7.77-7.79 (m, 1H), 7.82-7.84 (d, 1H), 8.21-8.23 (m, 1H). |
| 374 | 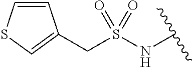 [SMILES: *NS(=O)(=O)Cc1ccsc1] | 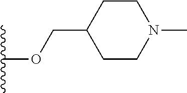 [SMILES: *OCC1CCN(C)CC1] | GS-2 | LC-MS: 617.1 [M + H]⁺; ¹HNMR (CD₃OD, 400 MHz): δ 1.47-1.49 (m, 2H), 1.60-1.70 (m, 1H), 1.82-1.90 (m, 3H), 2.78-2.82(m, 5H), 3.40-3.43 (d, 2H), 4.13 (s, 4H), 4.20 (s, 2H), 6.19 (s, 1H), 6.41 (s, 2H), 6.80-6.82 (d, 1H), 7.00 (s, 1H), 7.26-7.27 (t, 2H), 7.48 (s, |
| | | | | 1H), 7.54 (s, 1H), 7.57-7.61 (m, 1H), 7.86- |
| | | | | 7.88 (d, 1H), 8.2-8.24 (d, 1H). |
| 375 | 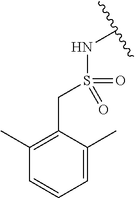 [SMILES: *NS(=O)(=O)Cc1c(C)cccc1C] | 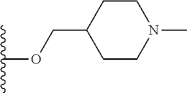 [SMILES: *OCC1CCN(C)CC1] | GS-2 | LC-MS: 639.5 [M + H]⁺; ¹HNMR (CD₃OD, 400 MHz): δ 1.49-1.55 (q, 2H), 1.81-1.88 (m, 3H), 2.18 (s, 6H), 2.72-2.76 (m, 5H), 3.34-3.39 (d, 2H), 4.12-4.14 (d, 4H), 4.36 (s, 2H), 6.33 (s, 1H), 6.40 (s, 2H), 6.95-6.97 (d, 2H), 7.03-7.07 (t, 1H), 7.27-7.29 (d, 1H), 7.43 (s, 1H), 7.52 (s, 1H), 7.56-7.63 (m, 3H), 7.79-7.81 (t, 1H), 7.87-7.89 (d, 1H), 8.22-8.24 (d, 1H). |
| 376 | 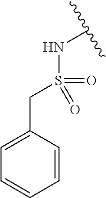 [SMILES: *NS(=O)(=O)Cc1ccccc1] | 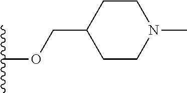 [SMILES: *OCC1CCN(C)CC1] | GS-2 | LC-MS: 611.2 [M + H]⁺; ¹HNMR (CD₃OD, 400 MHz): δ 1.42-1.52 (m, 2H), 1.82-1.90 (m, 3H), 2.75-2.81 (m, 5H), 3.38-3.41 (m, 2H), 4.11-4.15 (m, 6H), 6.26 (s, 1H), 6.42 (s, 2H), 7.04-7.06 (m, 2H), 7.20-7.29 (m, 4H), 7.42 (s, 1H), 7.55-7.63 (m, 4H), 7.82- 7.91 (m, 2H), 8.23-8.25 (m, 1H). |
| 377 | 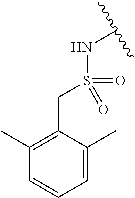 [SMILES: *NS(=O)(=O)Cc1c(C)cccc1C] | 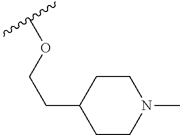 [SMILES: *OCCC1CCN(C)CC1] | GS-2 | LC-MS: 653.5 [M + H]⁺; ¹HNMR (CD₃OD, 400 MHz): δ 1.24-1.29 (m, 3H), 1.52-1.55 (d, 2H), 1.69-1.71 (d, 2H), 2.18 (s, 6H), 2.53-2.59 (t, 2H), 2.71 (s, 3H), 3.23-3.31 (m, 2H), 4.14 (s, 2H), 4.27-4.30 (t, 2H), 4.38 (d, 2H), 6.31-6.32 (s, 1H), 6.40 (s, 2H), 6.96- 6.97 (d, 2H), 7.04-7.08 (t, 1H), 7.27-7.29 (d, 1H), 7.42-7.44 (d, 1H), 7.57-7.79 (d, 1H), 7.57-7.64 (m, 3H), 7.80-7.83 (d, 1H), 7.87- 7.89 (d, 1H), 8.25-8.27 (d, 1H). |
| 378 | 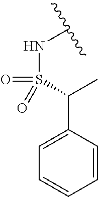 [SMILES: *NS(=O)(=O)[C@H](C)c1ccccc1] | 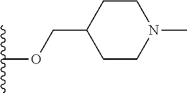 [SMILES: *OCC1CCN(C)CC1] | GS-2 | LC-MS: 625.5 [M + H]⁺; ¹HNMR (CD₃OD, 400 MHz): δ 1.53-1.55 (d, 5H), 1.81-1.90 (d, 3H), 2.77-2.82 (m, 5H), 3.38-3.41 (d, 2H), 4.00-4.04 (q, 1H), 4.14-4.15 (d, 4H), 6.28 (s, 1H), 6.35-6.47 (q, 2H), 7.07-7.09 (d, 2H), 7.19-7.30 (m, 5H), 7.55-7.64 (m, 4H), 7.78- 7.80 (d, 1H), 7.89-7.91 (d, 1H), 8.21-8.23 (d, 1H). |
| 379 | 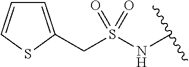 [SMILES: *NS(=O)(=O)Cc1cccs1] | 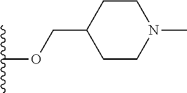 [SMILES: *OCC1CCN(C)CC1] | GS-2 | LC-MS: 617.4 [M + H]⁺; ¹HNMR (CD₃OD, 400 MHz): δ 1.43-1.52 (m, 2H), 1.81-1.89 (m, 3H), 2.75-2.80 (m, 5H), 3.34-3.38 (d, 2H), 4.11-4.15 (d, 4H), 4.36 (s, 2H), 6.20 (s, 1H), 6.41 (s, 2H), 6.77-6.79 (d, 1H), 6.87-6.88 (m, 1H), 7.29-7.33 (t, 2H), 7.50 (s, 1H), 7.54 (s, |
| | | | | 1H), 7.58-7.62 (m, 3H), 7.84-7.86 (m, 1H), |
| | | | | 7.87-7.91 (d, 1H), 8.22-8.24 (d, 1H). |
| 380 | 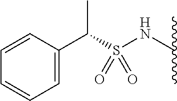 [SMILES: *NS(=O)(=O)[C@@H](C)c1ccccc1] | 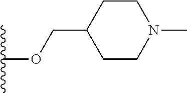 [SMILES: *OCC1CCN(C)CC1] | GS-2 | LC-MS: 625.5 [M + H]⁺; ¹HNMR (CD₃OD, 400 MHz): δ 1.49-1.55 (d, 5H), 1.80-1.89 (m, 3H), 2.76-2.82 (t, 5H), 3.38-3.41 (d, 2H), 4.02-4.04 (m, 1H), 4.13-4.14 (d, 4H), 6.28 (s, 1H), 6.34-6.47 (q, 2H), 7.07-7.09 (d, 2H), 7.19-7.24 (m, 3H), 7.25-7.31 (d, 2H), 7.55-7.61 |
| | | | | (m, 3H), 7.64 (s, 1H), 7.77-7.80 (m, 1H), |
| | | | | 7.89-7.7.91 (d, 1H), 8.21-8.23 (d, 1H). |
| 381 | 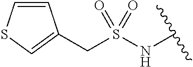 [SMILES: *NS(=O)(=O)Cc1ccsc1] | 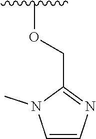 [SMILES: *OCc1nccn1C] | GS-2 | LC-MS: 598.1 [M − H]⁻; ¹HNMR (CD₃OD, 400 MHz): δ 3.80 (s, 3H), 4.12 (s, 4H), 5.51 (s, 2H), 6.14 (s, 1H), 6.35 (s, 2H), 6.78-6.80 (m, 1H), 6.94-6.95 (m, 1H), 7.21-7.29 (m, 2H), 7.37-7.42 (m, 3H), 7.53-7.59 (m, 3H), 7.71(s, 1H), 7.78-7.82 (m, 1H), 7.88-7.90 (m, 1H), 8.14-8.17 (m, 1H). |
| 382 | 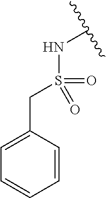 [SMILES: *NS(=O)(=O)Cc1ccccc1] | 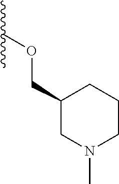 [SMILES: *OC[C@H]1CCCN(C)C1] | GS-2 | LC-MS: 611.1 [M + H]⁺; ¹HNMR (CD₃OD, 400 MHz): δ 1.16-1.18 (q, 1H), 1.66-1.81 (m, 3H), 2.23 (m, 1H), 2.52-2.55 (t, 2H), 2.67 (s, 3H), 3.36-3.42 (m, 2H), 4.07 (s, 2H), 4.13 (s, 2H), 4.16-4.20 (m, 1H), 4.25- 4.29 (m, 1H), 6.283-6.286 (d, 1H), 6.43 (s, 2H), 7.02-7.04 (d, 2H), 7.20-7.24 (t, 2H), 7.27-7.29 (t, 2H), 7.39-7.40 (d, 1H), 7.54 (s, 1H), 7.59-7.63 (m, 2H), 7.68 (s, 1H), 7.82- 7.92 (m, 1H), 7.90-7.92 (d, 1H), 8.24-8.26 (d, 1H). |
| 383 | 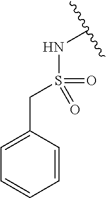 [SMILES: *NS(=O)(=O)Cc1ccccc1] | 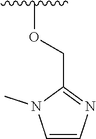 [SMILES: *OCc1nccn1C] | GS-2 | LC-MS: 594.0 [M + H]⁺; ¹HNMR (CD₃OD, 300 MHz): δ 3.77 (s, 3H), 4.04 (s, 2H), 4.12 (s, 2H), 5.50 (s, 2H), 6.20 (s, 1H), 6.361 (s, 2H), 6.98-7.00 (d, 2H), 7.15-7.19 (t, 2H), 7.22-7.26 (m, 2H), 7.35 (s, 2H), 7.38 (s, 1H), 7.54-7.57 (m, 3H), 7.72 (s, 1H), 7.78- 7.80 (m, 1H), 7.89-7.91 (d, 1H), 8.15-8.17 (m, 1H). |
| 384 | 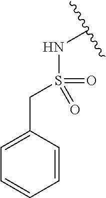 [SMILES: *NS(=O)(=O)Cc1ccccc1] | 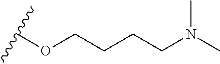 [SMILES: *OCCCCN(C)C] | GS-2 | LC-MS: 599.4 [M + H]⁺; ¹HNMR (CD₃OD, 400 MHz): δ 1.68-1.70 (m, 2H), 2.74 (s, 6H), 3.16-3.17 (m, 3H), 3.50-3.52 (m, 3H), 4.08 (s, 2H), 4.12 (s, 2H), 4.28-4.30 (m, 2H), 5.90 (s, 1H), 6.44 (s, 2H), 7.03-7.05 (d, 2H), 7.20-7.30 (m, 3H), 7.40 (s, 1H), 7.50 (s, 1H), 7.60-7.61 (m, 2H), 7.79-7.80 (m, 1H), 7.90-7.91 (d, 1H), 8.19-8.20 (d, 1H). |
| 385 | 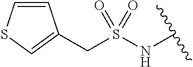 [SMILES: *NS(=O)(=O)Cc1ccsc1] | 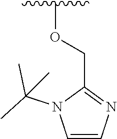 [SMILES: *OCc1nccn1C(C)(C)C] | GS-2 | LC-MS: 641.9 [M + H]⁺; ¹HNMR (CD₃OD, 400 MHz): δ 1.68 (s, 9H), 4.12 (s, 4H), 5.52 (s, 2H), 6.14 (s, 1H), 6.37 (s, 2H), 6.79-6.81 (d, 1H), 6.94-6.94 (d, 1H), 7.21-7.23 (m, 1H), 7.25-7.27(d, 1H), 7.40-7.41 (d, 1H), 7.52 (s, 2H), 7.55-7.58 (m, 2H), 7.59 (s, 1H), 7.79-7.82 (m, 1H), 7.86-7.88(d, 1H), 8.14-8.16 (m, 1H), 8.83 (s, 1H). |
| 386 | 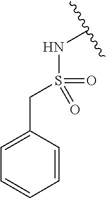 [SMILES: *NS(=O)(=O)Cc1ccccc1] | 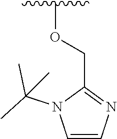 [SMILES: *OCc1nccn1C(C)(C)C] | GS-2 | LC-MS: 636.1 [M + H]⁺; ¹HNMR (CD₃OD, 400 MHz): δ 1.66 (s, 9H), 4.03 (s, 2H), 4.11 (s, 2H), 5.52 (s, 2H), 6.20 (s, 1H), 6.38 (s, 2H), 6.98-7.00 (d, 2H), 7.15-7.18 (m, 2H), 7.21-7.26 (m, 2H), 7.35 (s, 1H), 7.50-7.60 (m, 5H), 7.78-7.79 (m, 1H), 7.87-7.89 (d, 1H), 8.15-8.16 (m, 1H), 8.21 (s, 1H). |
| 387 | 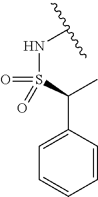 [SMILES: *NS(=O)(=O)[C@@H](C)c1ccccc1] | 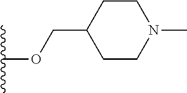 [SMILES: *OCC1CCN(C)CC1] | GS-2 | LC-MS: 625.5 [M + H]⁺; ¹HNMR (CD₃OD, 400 MHz): δ 1.53-1.55 (d, 5H), 1.82-1.90 (d, 3H), 2.77-2.82 (m, 5H), 3.34-3.41 (d, 2H), 4.00-4.03 (q, 1H), 4.14-4.15 (d, 4H), 6.28 (s, 1H), 6.35-6.47 (q, 2H), 7.07-7.09 (d, 2H), 7.19-7.30 (m, 5H), 7.55-7.64 (m, 4H), 7.78- 7.80 (d, 1H), 7.89-7.91 (d, 1H), 8.21-8.24 (d, 1H). |
| 388 | 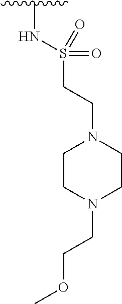 [SMILES: *NS(=O)(=O)CCN1CCN(CCOC)CC1] | 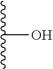 [SMILES: *O] | GS-1 | LC-MS: 580.1 [M + H]⁺; ¹HNMR (CD₃OD, 400 mHz): δ 2.12-2.5 (m, 4H), 2.72 (m, 4H), 2.80-3.00 (m, 2H), 3.10 (s, 2H), 3.19 (t, 3H), 3.36 (s, 2H), 3.60 (d, 2H), 4.14 (s, 2H), 6.28 (s, 1H), 6.42 (s, 2H), 7.26 (d, 1H), 7.49 (t, 3H), 7.58-7.60 (m, 2H), 7.86 (d, 2H), 8.22- 8.23 (d, 1H). |
| 389 | 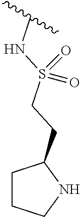 [SMILES: *NS(=O)(=O)CC[C@@H]1CCCN1] | 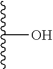 [SMILES: *O] | GS-1 | LC-MS: 507 [M + H]⁺; ¹HNMR (CD₃OD, 300 MHz): δ 1.92-2.15 (m, 6H), 3.06-3.12 (m, 2H), 3.22-3.34 (m, 2H), 3.49-3.54 (m, 1H), 4.13 (s, 2H), 6.23 (s, 1H), 6.39 (s, 2H), 7.23-7.27 (d, 1H), 7.45 (s, 1H), 7.52-7.59 (m, 4H), 7.82-7.86 (m, 2H), 8.18-8.22 (d, 1H). |
| 390 | 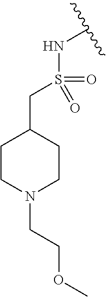 [SMILES: *NS(=O)(=O)CC1CCN(CCOC)CC1] | 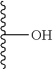 [SMILES: *O] | GS-1 | LC-MS: 565.1 [M + H]⁺; ¹HNMR (CD₃OD, 400 MHz): δ 1.52 (m, 2H), 2.00-2.04 (d, 3H), 2.94-2.96 (m, 4H), 3.30-3.34 (m, 2H), 3.39 (s, 3H), 3.53-3.56 (d, 2H), 3.68 (s, 2H), 4.13(s, 2H), 6.22 (s, 1H), 6.40 (s, 2H), 7.24- 7.26 (d, 1H), 7.45-7.59 (m, 5H), 7.83-7.87 (m, 2H), 8.20-8.22 (d, 1H) |
| 391 | 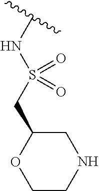 [SMILES: *NS(=O)(=O)C[C@H]1CNCCO1] | 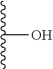 [SMILES: *O] | GS-1 | LC-MS: 509 [M + H]⁺; ¹HNMR (CD₃OD, 300 MHz): δ 2.85 (t, 1H), 3.03 (m, 1H), 3.15- 3.17 (m, 3H), 3.23-3.31 (m, 1H), 3.57 (t, 1H), 3.77 (dd, 1H), 4.09-4.13 (m, 3H), 6.19 (s, 1H), 6.38 (d, 1H), 6.45 (d, 1H), 7.24 (d, 1H), 7.44 (s, 1H), 7.25 (s, 1H), 7.57-7.59 (m, 2H), 7.60-7.62 (m, 1H), 7.85 (d, 2H), 12.33- 12.34 (d, 1H). |
| 392 | 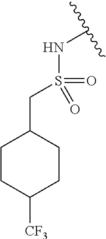 [SMILES: *NS(=O)(=O)CC1CCC(C(F)(F)F)CC1] | 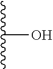 [SMILES: *O] | GS-1 | LC-MS: 573.9 [M + H]⁺; ¹HNMR (CD₃OD, 400 MHz): δ 1.34-1.65 (m, 9H), 2.08-2.11 (m, 1H), 2.89-2.91 (d, 2H), 4.10 (s, 2H), 6.20-6.21 (d, 1H), 6.41 (s, 2H), 7.23-7.25 (t, 1H), 7.450-7.58 (m, 5H), 7.81-7.86 (q, 2H), 8.18-8.22 (t, 1H). |
| 393 | 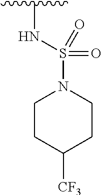 [SMILES: *NS(=O)(=O)N1CCC(C(F)(F)F)CC1] | 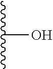 [SMILES: *O] | GS-1 | LC-MS: 561.0 [M + H]⁺; ¹HNMR (CD₃OD, 400 mHz): δ 1.24-1.34 (m, 2H), 1.72 (d, 2H), 2.07-2.08 (m. 1H), 2.42-2.48 (m, 2H), 3.51- 3.52 (d, 2H), 4.14 (s, 2H), 6.36 (s, 1H), 6.43 (s, 2H), 7.25-7.27 (m, 1H), 7.39 (d, 1H), 7.46 (s. 1H), 7.54-7.58 (m, 3H), 7.80-7.83 (m, 1H), 7.88 (d, 1H), 8.20-8.22 (m, 1H). |

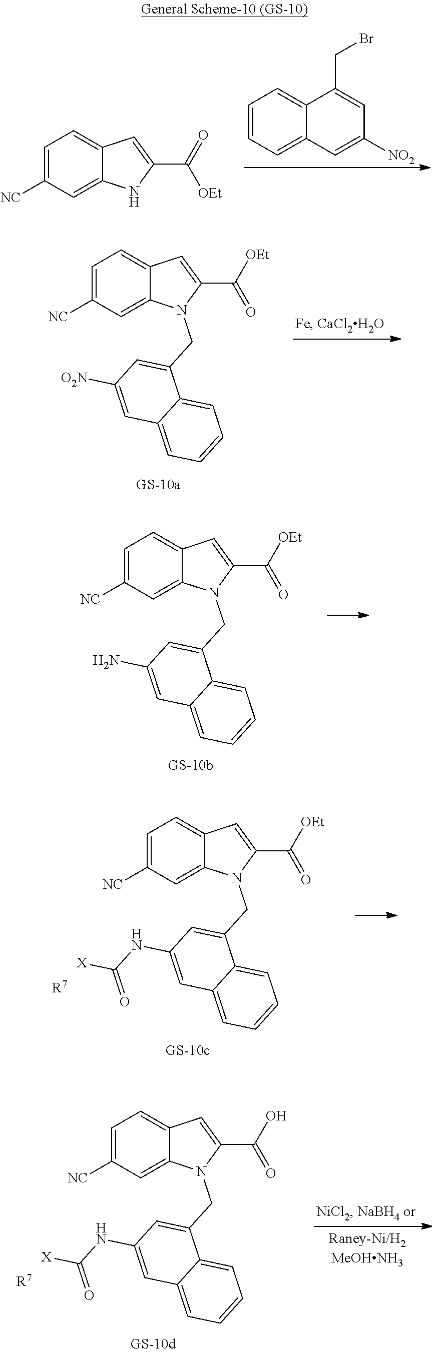

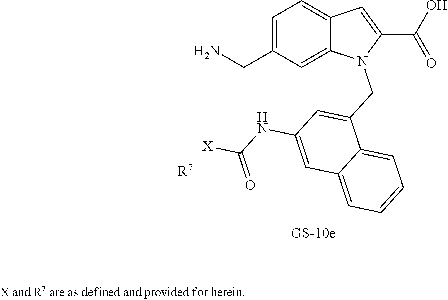

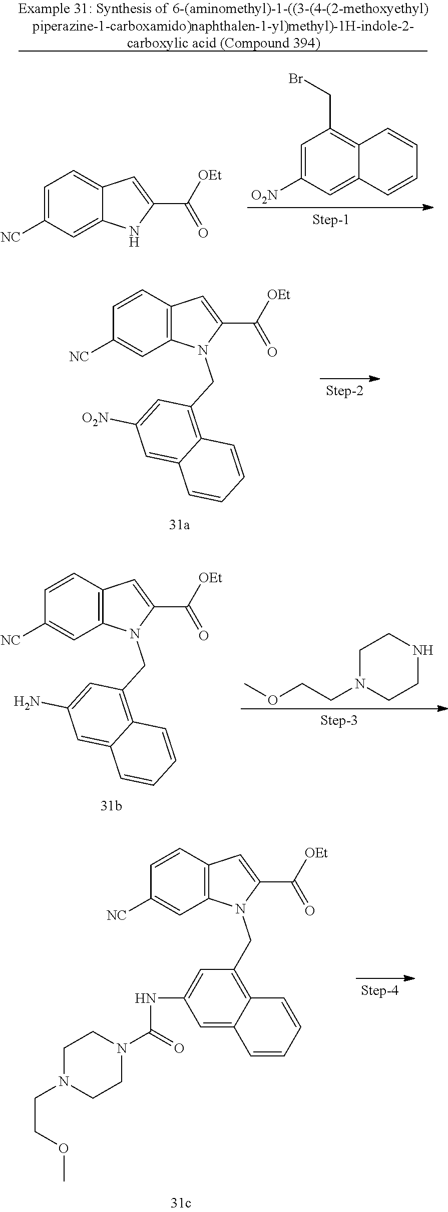

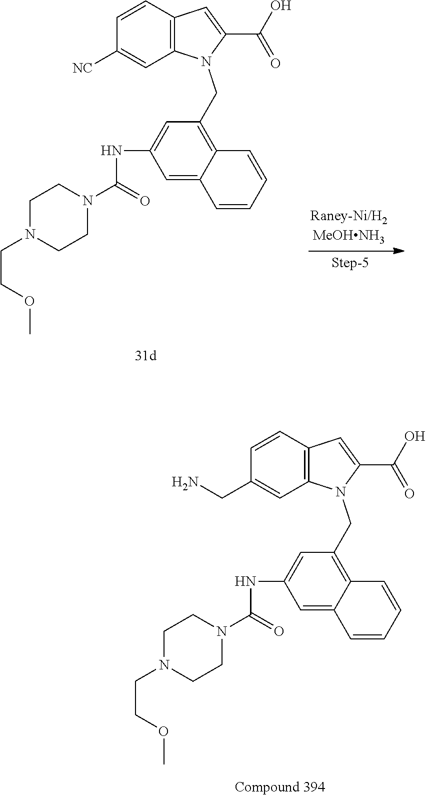 [SMILES: COCCN1CCN(C(=O)Nc2cc(Cn3c(C(=O)O)cc4ccc(C#N)cc43)c3ccccc3c2)CC1 ; COCCN1CCN(C(=O)Nc2cc(Cn3c(C(=O)O)cc4ccc(CN)cc43)c3ccccc3c2)CC1]

### Step-1: Synthesis of ethyl 6-cyano-1-((3-nitronaphthalen-1-yl)methyl)-1H-indole-2-carboxylate

To a stirred solution of ethyl 6-cyano-1H-indole-2-carboxylate (1.45 g, 6.77 mmol) in DMF (15 mL) was added Cs₂CO₃ (6.6 g, 20.3 mmol) followed by 1-(bromomethyl)-3-nitronaphthalene (2.2 g, 8.13 mmol) and stirred at RT for 16 h at RT. The reaction mixture was then poured into ice-cold water to form a solid that was filtered and dried. This was washed with diethyl ether to get pure title compound 31a (1.7 g, crude). LC-MS: 398.0 [M−H].

### Step-2: Synthesis of ethyl 1-((3-aminonaphthalen-1-yl)methyl)-6-cyano-1H-indole-2-carboxylate

Compound ethyl 6-cyano-1-((3-nitronaphthalen-1-yl)methyl)-1H-indole-2-carboxylate (1.2 g, 3.0 mmol) was dissolved in ethanol (12 mL), THF (12 mL) and to the solution was added Pd—C (10% load) (0.4 g, 30% W/W) and stirred under positive pressure of hydrogen using a bladder for 48 h. The reaction mass was filtered through Celite, and the filtrate was concentrated to yield the title compound 31b (1.7 g, crude). LC-MS: 370.10 [M+H]⁺.

### Step-3: Synthesis of ethyl 6-cyano-1-(3-(4-(2-methoxyethyl)piperazine-1-carboxamido)naphthalen-1-yl)methyl)-1H-indole-2-carboxylate

A stirred solution of triphosgene (0.084 g, 0.312 mmol) in THF (25 mL) was cooled to 0° C. and to the solution was added ethyl 1-((3-aminonaphthalen-1-yl)methyl)-6-cyano-1H-indole-2-carboxylate (0.35 g, 0.947 mmol) and DIPEA (0.366 g, 2.84 mmol). The mixture was stirred for 30 minutes, then 1-(2-methoxyethyl)piperazine (0.136 g, 0.0.947 mmol) and DMAP (0.08 g, 0.66 mmol) were added at RT. The reaction mixture was quenched with methanol and concentrated. The residue was purified by Combi flash chromatography using 30% ethyl acetate in hexane as eluent to yield the title compound 31c. LC-MS: 540.25 [M+H]⁺.

### Step-4: Synthesis of 6-cyano-1-((3-(4-(2-methoxyethyl)piperazine-1-carboxamido)naphthalen-1-yl)methyl)-1H-indole-2-carboxylic acid

The experimental procedure was the same as described in Step-3 of Example-1 with appropriate variations in methods, reactants, quantities of reagents, and solvents to obtain the compound 31d (0.35 g, crude). LC-MS: 512.15 [M+H]⁺.

### Step-5: Synthesis of 6-(aminomethyl)-1-((3-(4-(2-methoxyethyl)piperazine-1-carboxamido)naphthalen-1-yl)methyl)-1H-indole-2-carboxylic acid (Compound 394)

The experimental procedure was the same as described in Step-4 of Example-1 with appropriate variations in methods, reactants, quantities of reagents, and solvents to obtain the title compound (Compound 394) (0.15 g). (Prep HPLC method: Column-ZORBAX (250 mm×21.2 mm), flow rate-20 mL/min, mobile phase-A was 0.1% TFA in water, B was acetonitrile, Gradient program-5% B at 0 minute, 10% B at 2 minutes, 50% B at 10 minutes). LC-MS: 516.2 [M+H]*; ¹HNMR (DMSO-d₆, 400 MHz): δ 2.90-3.00 (bs, 2H), 3.02-3.20 (bs, 2H), 3.40-3.70 (bs, 9H), 4.00-4.20 (bs, 4H), 6.20 (s, 2H), 6.40 (s, 1H), 7.20-7.21 (d, 1H), 7.40 (s, 1H), 7.45-7.60 (m, 3H), 7.80-7.82 (d, 2H), 7.90 (s, 1H), 8.09-8.19 (m, 2H), 8.80 (s, 1H), 9.80 (bs, 1H), 13.00 (bs, 1H).

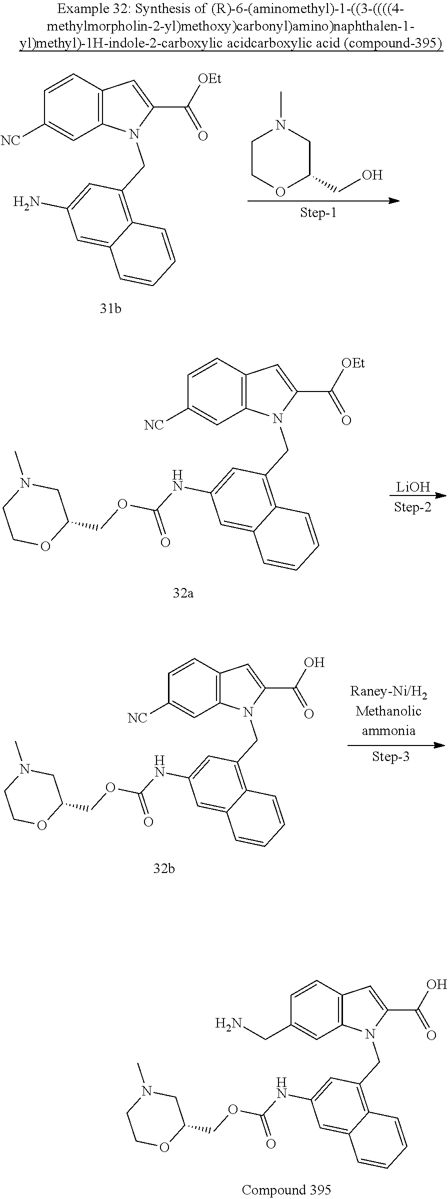

### Step-1: Synthesis of ethyl (R)-6-cyano-1-(3-((((4-methylmorpholin-2-yl)methoxy)carbonyl)amino)naphthalen-1-yl)methyl)-1H-indole-2-carboxylate

An ice-cold solution of triphosgene (0.06 g, 0.22 mmol) THF (25 mL) was added DIPEA (0.49 g, 2.02 mmol) followed by ethyl 1-((3-aminonaphthalen-1-yl)methyl)-6-cyano-1H-indole-2-carboxylate (0.25 g, 0.67 mmol) in THF (10 mL). The reaction mixture was stirred at RT in 30 minutes. Then added (R)-(4-methylmorpholin-2-yl)methanol (0.106 g, 0.810 mmol) in THE. The resultant mixture was stirred for 2 h and concentrated. The crude compound was purified by Combi flash column chromatography using 5% methanol in DCM to get the pure title compound 32a (0.094 g. crude). LC-MS: 527.2 [M+H]⁺.

### Step-2: Synthesis of (R)-6-cyano-1-(3-((((4-methylmorpholin-2-yl)methoxy)carbonyl)amino)naphthalen-1-yl)methyl)-1H-indole-2-carboxylic acid

The experimental procedure was the same as described in Step-3 of Example-1 with appropriate variations in methods, reactants, quantities of reagents, and solvents to obtain the compound 32b (0.103 g, crude). LC-MS: 499.2 [M+H]⁺.

### Step-3: Synthesis of (R)-6-(aminomethyl)-1-((3-((((4-methylmorpholin-2-yl)methoxy)carbonyl)amino)naphthalen-1-yl)methyl)-1H-indole-2-carboxylic acid carboxylic acid

The experimental procedure was the same as described in Step-4 of Example-1 with appropriate variations in methods, reactants, quantities of reagents, and solvents to obtain the title compound (Compound 395) (0.043 g). (Prep HPLC method: Column-ZORBAX (150 mm×20 mm), flow rate-20 mL/min, mobile phase-A was 0.1% TFA in water, B was acetonitrile, Gradient program-10% B at 0 minute, 20% B at 2 minutes, 40% B at 8 minutes).

LC-MS: 503.20 [M+H]⁺; ¹HNMR (CD₃OD, 400 MHz): δ 2.91 (s, 3H), 2.91-2.98 (m, 2H), 3.0-3.1 (m, 1H), 3.40-3.49 (m, 2H), 3.8 (t, 1H), 3.9 (bs, 1H), 4.08-4.17 (m, 4H), 6.33 (s, 1H), 6.43 (s, 2H), 7.24-7.26 (d, 1H), 7.43 (s, 1H), 7.52-7.56 (m, 3H), 7.81-7.85 (m, 2H), 7.94 (s, 1H), 8.18 (s, 1H).

The following compounds listed in Table 7 were prepared according to GS-10 by following the similar procedures as described above for Example-31 or Example-32 using appropriate reagents with suitable modifications known to the one skilled in the art.

TABLE 7

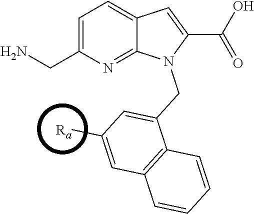

| Compound | Ra | Method | Spectral data |
|---|---|---|---|
| 396 | 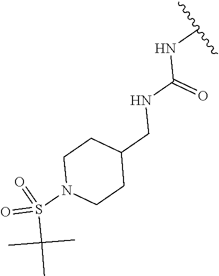 [SMILES: *NC(=O)NCC1CCN(S(=O)(=O)C(C)(C)C)CC1] | | LC-MS: 606.0 [M + H]⁺; ¹HNMR (CD₃OD, 400 MHz): δ 1.17-1.20 (m, 2H), 1.33 (s, 9H), 1.60- 1.68 (m, 2H), 2.91-2.95 (m, 2H), 3.02-3.04 (m, 2H), 3.45-3.55 (m, 1H), 3.77-3.80 (m, 2H), 4.13 (s, 2H), 6.08 (s, 1H), 6.42 (s, 2H), 7.23-7.25 (d, 1H), 7.45- 7.52 (m, 4H), 7.80-7.92 (m, 3H), 8.14-8.16 (m, 1H). |
| 397 | 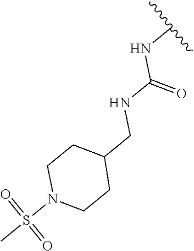 [SMILES: *NC(=O)NCC1CCN(S(C)(=O)=O)CC1] | | LC-MS: 564.2 [M + H]⁺; ¹HNMR (CD₃OD, 400 MHz): δ 1.15-1.30 (m, 2H), 1.50-1.60 (m, 1H), 1.70-1.75 (m, 2H), 2.60-2.70 (m, 2H), 2.80 (s, 3H), 3.04-3.05 (d, 2H), 3.60-3.75 (m, 2H), 4.13 (s, 2H), 6.10 (s, 1H), 6.42 (s, 2H), 7.20-7.25 (d, 1H), 7.44- 7.50 (m, 4H), 7.75-7.95 (m, 3H), 8.10-8.15 (m, 1H). |
| 398 | 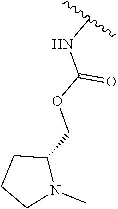 [SMILES: *NC(=O)OC[C@H]1CCCN1C] | | LC-MS : 487.5[M + H]⁺; ¹HNMR (CD₃OD, 300 MHz): δ 1.90-2.20 (m, 3H), 2.21-2.30 (m, 1H), 2.29 (s, 3H), 3.60-3.70 (m, 3H), 4.10-4.20 (m, 3H), 4.40-4.51 (m, 1H), 6.30 (s, 1H), 6.40 (s, 2H), 7.21- 7.24 (d, 1H), 7.41 (s, 1H), 7.50-7.55 (m, 3H), 7.80- 7.85 (m, 2H), 7.99 (s, 1H), 8.16-8.18 (m. 1H). |

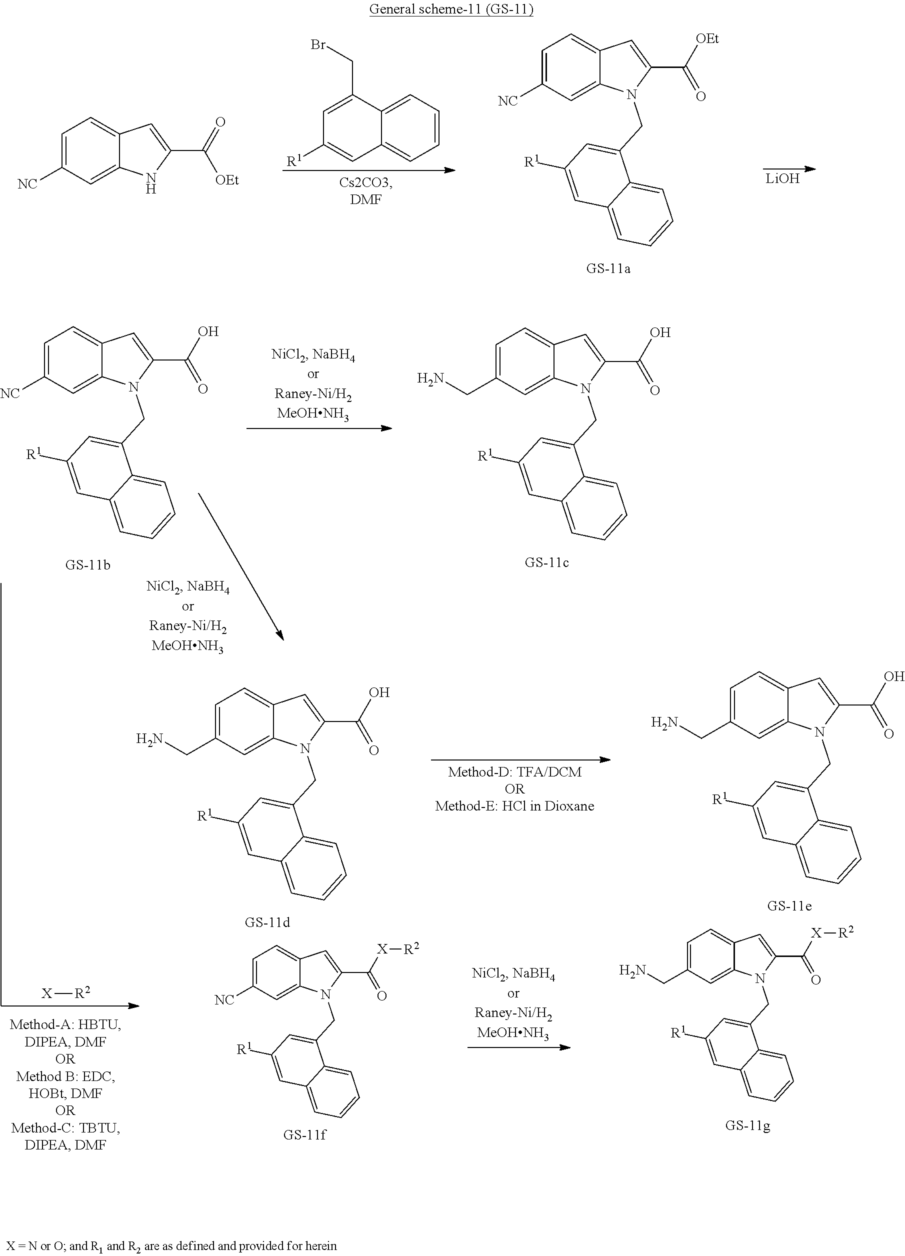

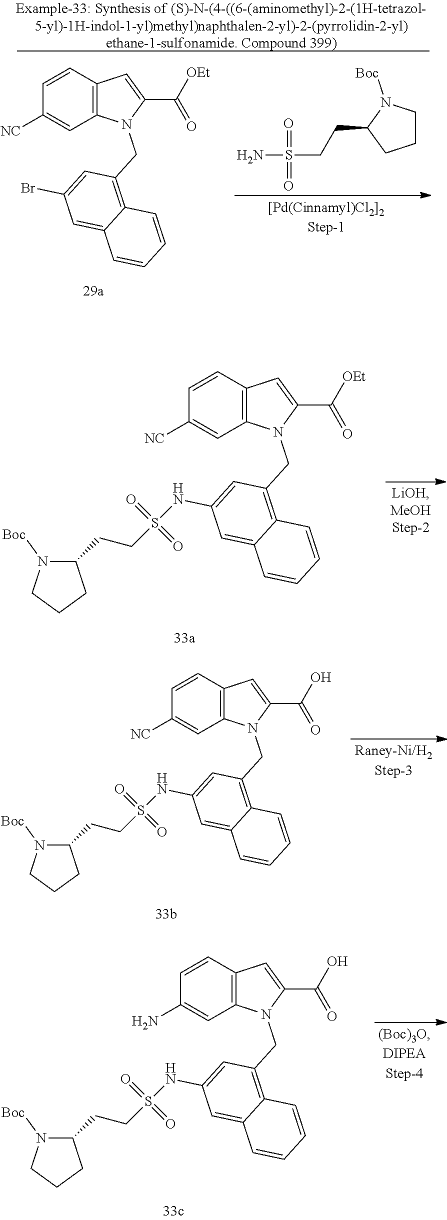 [SMILES: CCOC(=O)c1cc2ccc(C#N)cc2n1Cc1cc(Br)cc2ccccc12 ; CC(C)(C)OC(=O)N1CCC[C@H]1CCS(N)(=O)=O ; CCOC(=O)c1cc2ccc(C#N)cc2n1Cc1cc(NS(=O)(=O)CC[C@@H]2CCCN2C(=O)OC(C)(C)C)cc2ccccc12 ; CC(C)(C)OC(=O)N1CCC[C@H]1CCS(=O)(=O)Nc1cc(Cn2c(C(=O)O)cc3ccc(C#N)cc32)c2ccccc2c1 ; CC(C)(C)OC(=O)N1CCC[C@H]1CCS(=O)(=O)Nc1cc(Cn2c(C(=O)O)cc3ccc(N)cc32)c2ccccc2c1]

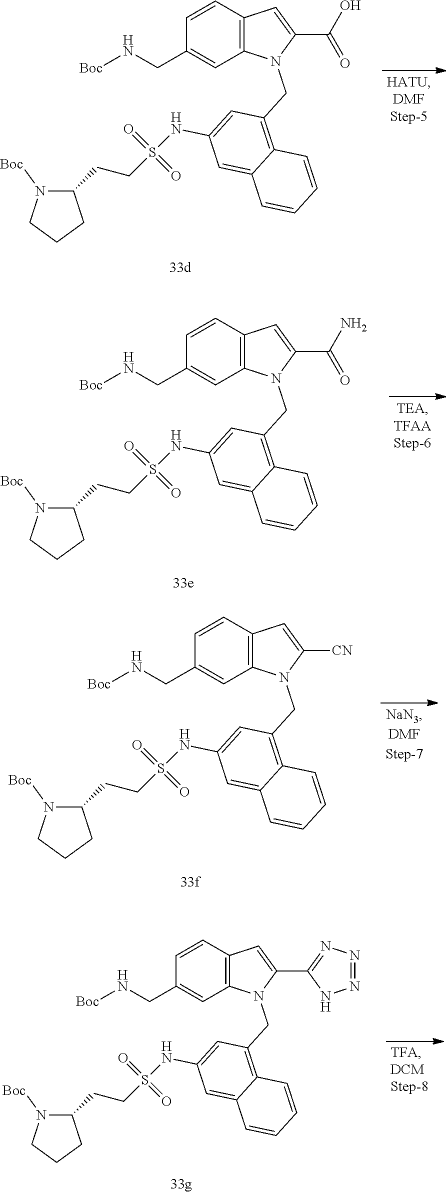 [SMILES: CC(C)(C)OC(=O)NCc1ccc2cc(C(=O)O)n(Cc3cc(NS(=O)(=O)CC[C@@H]4CCCN4C(=O)OC(C)(C)C)cc4ccccc34)c2c1 ; CC(C)(C)OC(=O)NCc1ccc2cc(C(N)=O)n(Cc3cc(NS(=O)(=O)CC[C@@H]4CCCN4C(=O)OC(C)(C)C)cc4ccccc34)c2c1 ; CC(C)(C)OC(=O)NCc1ccc2cc(C#N)n(Cc3cc(NS(=O)(=O)CC[C@@H]4CCCN4C(=O)OC(C)(C)C)cc4ccccc34)c2c1 ; CC(C)(C)OC(=O)NCc1ccc2cc(-c3nnn[nH]3)n(Cc3cc(NS(=O)(=O)CC[C@@H]4CCCN4C(=O)OC(C)(C)C)cc4ccccc34)c2c1]

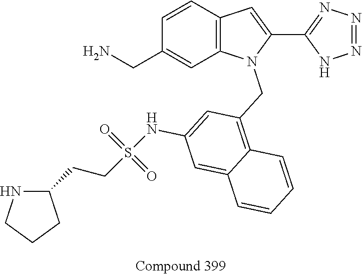 [SMILES: NCc1ccc2cc(-c3nnn[nH]3)n(Cc3cc(NS(=O)(=O)CC[C@@H]4CCCN4)cc4ccccc34)c2c1]

### Step-1: Synthesis of ethyl(S)-1-((3-((2-(1-(tert-butoxycarbonyl)pyrrolidin-2-yl)ethyl)sulfonamido)naphthalen-1-yl)methyl)-6-cyano-1H-indole-2-carboxylate

A solution of ethyl 1-((3-bromonaphthalen-1-yl)methyl)-6-cyano-1H-indole-2-carboxylate (1 g, 2.3 mmol), tert-butyl(S)-2-(2-sulfamoylethyl)pyrrolidine-1-carboxylate (0.77 g, 2.7 mmol) in 1,4-Dioxane (20 ml) was purged with nitrogen, added K₃PO₄ (1.4 g, 6.6 mmol) followed by Palladium Cinnamyl Chloride dimer (0.12 g, 0.23 mmol) and tert butyl-XPhos (0.2 g, 0.17 mmol). After addition, the mixture was stirred at 90° C. for 12 h. The reaction mixture was added water and extracted with 15% methanol in DCM, the organic portion was dried over Na₂SO₄ and concentrated to produce crude produce. The crude compound was purified by Combi flash column chromatography. By eluting with 30% EtOAc in Hexane. This afforded the crude product 33a, which was used for the next step without further purification (1 g, crude).

### Step-2: Synthesis of ethyl(S)-6-(aminomethyl)-1-((3-((2-(1-(tert-butoxycarbonyl)pyrrolidin-2-yl)ethyl)sulfonamido)naphthalen-1-yl)methyl)-1H-indole-2-carboxylate

The experimental procedure was the same as described in Step-3 of Example-1 with appropriate variations in methods, reactants, quantities of reagents, and solvents to obtain the compound 33b (0.75 g, crude). LC-MS: 601.3 [M−H]⁻.

### Step-3: Synthesis of ethyl(S)-6-(((tert-butoxycarbonyl)amino)methyl)-1-((3-((2-(1-(tert-butoxycarbonyl)pyrrolidin-2-yl)ethyl)sulfonamido)naphthalen-1-yl)methyl)-1H-indole-2-carboxylate

The experimental procedure was the same as described in Step-4 of Example-1 with appropriate variations in methods, reactants, quantities of reagents, and solvents to obtain the compound 33c (0.65 g, crude).

### Step-4: Synthesis of(S)-6-(((tert-butoxycarbonyl)amino)methyl)-1-((3-((2-(1-(tert-butoxycarbonyl)pyrrolidin-2-yl)ethyl)sulfonamido)naphthalen-1-yl)methyl)-1H-indole-2-carboxylic acid

The experimental procedure was the same as described in Step-3 of Example-4 with appropriate variations in methods, reactants, quantities of reagents, and solvents to obtain the compound 33d (0.5 g, crude). LC-MS: 707.40 [M+H]⁺.

### Step-5: Synthesis of tert-butyl(S)-2-(2-(N-(4-(6-(((tert-butoxycarbonyl)amino)methyl)-2-carbamoyl-1H-indol-1-yl)methyl)naphthalen-2-yl) sulfamoyl)ethyl)pyrrolidine-1-carboxylate

The experimental procedure was the same as described in Step-2 of Example-4 with appropriate variations in methods, reactants, quantities of reagents, and solvents to obtain the compound 33e (0.3 g, crude). LC-MS: 704.20 [M−H]⁻.

### Step-6: Synthesis of tert-butyl(S)-2-(2-(N-(4-((6-(((tert-butoxycarbonyl)amino)methyl)-2-cyano-1H-indol-1-yl)methyl)naphthalen-2-yl) sulfamoyl)ethyl)pyrrolidine-1-carboxylate

A solution of tert-butyl(S)-6-(((tert-butoxy carbonyl)amino)methyl)-1-((3-((2-(1-(tert-butoxycarbonyl)pyrrolidin-2-yl)ethyl)sulfonamido)naphthalen-1-yl)methyl)-1H-indole-2-carboxylic acid (0.3 g, 0.425 mmol) in DCM (8 mL) was cooled to 0° C. and to the solution was added triethylamine (0.129 g, 0.1.27 mmol), trifluoroacetic anhydride (0.178 g, 0.84 mmol). After stirring at RT for 2 h, the reaction mixture was diluted with DCM, washed with sat. NaHCO₃, dried over Na₂SO₄ and concentrated. This afforded the title compound 33f (0.04 g, crude).

### Step-7: Synthesis of tert-butyl(S)-2-(2-(N-(4-((6-(((tert-butoxycarbonyl)amino)methyl)-2-(1H-tetrazol-5-yl)-1H-indol-1-yl)methyl)naphthalen-2-yl) sulfamoyl)ethyl)pyrrolidine-1-carboxylate

A mixture of tert-butyl(S)-2-(2-(N-(4-((6-(((tert-butoxycarbonyl)amino)methyl)-2-cyano-1H-indol-1-yl)methyl)naphthalen-2-yl) sulfamoyl)ethyl)pyrrolidine-1-carboxylate (0.07 g, 0.10 mmol), NH₄Cl (0.049 g, 0.40 mmol) and NaN3 (0.026 g, 0.39 mmol) in DMF (3 mL) was heated to 110° C. for 15 h. The reaction mixture was concentrated and washed with diethyl ether to yield the title compound which was used as such in the next step without any purification to obtain the compound 33g (0.1 g, crude). LC-MS: 729.40 [M−H]⁻.

### Step-8: Synthesis of(S)—N-(4-((6-(aminomethyl)-2-(1H-tetrazol-5-yl)-1H-indol-1-yl)methyl)naphthalen-2-yl)-2-(pyrrolidin-2-yl) ethane-1-sulfonamide. (Compound 399)

The experimental procedure was the same as described in Step-5 of Example-1 with appropriate variations in methods, reactants, quantities of reagents, and solvents to obtain the title compound (Compound 399) (0.025 g, 34%).

LC-MS: 531.3 [M+H]⁺; ¹HNMR (CD₃OD, 400 MHz): δ 1.40-1.46 (t, 1H), 1.89-2.01 (m, 6H), 3.24-3.29 (m, 2H), 3.40-3.42 (d, 1H), 4.10 (s, 2H), 6.33 (s, 2H), 6.48 (s, 2H), 7.25-7.28 (d, 1H), 7.32 (s, 1H), 7.50-7.60 (m, 4H), 7.84-7.86 (d, 2H), 8.21-8.23 (d, 1H).

### Biological Activity Testing:

Method I: Test compounds were evaluated for their potential to inhibit Human Recombinant Matriptase 2 (in-house) using fluorescence based assay. The concentration of Recombinant Matriptase 2 and the substrate used in the assay was: 3.5 nM (In-house Matriptase 2) and 62 μM (Boc-Gln-Ala-Arg-7-amido-4-methyl coumarin hydrobromide −Cat log: I-1550 from Bachem) respectively. The assay buffer used was 50 mM TRIS pH 7.5, 150 mM NaCl, 0.01% Gelatin TCL059 Himedia). The assay was performed using 384 well black flat bottom plate from grenier (Cat log: 781076) at 25° C. The enzyme and compound were preincubated for 30 mins, the plate was read at 60 mins after substrate addition at wavelength Ex: 360/Em: 480 nm. The final assay volume was 20 μl. A stock solution of compounds was initially prepared in DMSO and appropriate dilutions were made and IC₅₀ was determined for all the compounds (final DMSO conc in the assay was 1%). All the measurements were carried out using the Spectramax M5, Molecular devices.

To determine Kᵢ values, dose response curves were generated by plotting percentage inhibition as a function of inhibitor concentration and the data was fitted to sigmoidal non-linear regression equation (variable slope) using Graph Pad prism software V8.

Table A, Table B. and Table C list Kᵢ values for certain exemplary compounds.

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists. e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permit the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound having a formula of 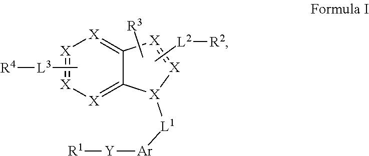
or a pharmaceutically acceptable salt thereof,
wherein:
Ar is an optionally substituted ring selected from phenyl, a 5-6 membered heteroaromatic ring having 1-4 heteroatoms independently selected from nitrogen, oxygen, and sulfur, an 8-10 membered bicyclic aromatic carbocyclic ring, and an 8-10 membered bicyclic heteroaromatic ring having 1-4 heteroatoms independently selected from nitrogen, oxygen, and sulfur;
each X is independently CH, C-L³-R⁴, C-L²-R², C—R³, or N;
Y is a bond, —NR⁵—, CR¹⁰R^{10′}, or —O—;
L¹ is a bond, or an optionally substituted bivalent C₁₋₈ saturated or unsaturated, straight or branched hydrocarbon chain, wherein 1, 2, or 3 methylene units of the hydrocarbon chain are optionally and independently replaced by —S(O)₂—, —C(O)—, —NH—, or —O—;
R¹ is H, D, R⁷, R⁷SO₂—, R⁷S(O)—, R⁷C(O)—, R⁷NR—C(O)—, or R⁷O—C(O)—;
R⁷ is R¹¹(CR¹⁰R^{10′})ₙ—;
R¹¹ is H, an optionally substituted C₁₋₆ alkyl, or an optionally substituted ring selected from phenyl, a 5-6 membered heteroaromatic ring having 1-4 heteroatoms independently selected from nitrogen, oxygen, and sulfur, a 3-7 membered monocyclic carbocyclic ring, a 4-7 membered heterocyclic ring having 1-3 heteroatoms independently selected from nitrogen, oxygen, and sulfur, a 5-10 membered bicyclic carbocyclic ring, a 7-10 membered bicyclic heterocyclic ring having 1-4 heteroatoms independently selected from nitrogen, oxygen, and sulfur, an 8-10 membered bicyclic aromatic carbocyclic ring, and an 8-10 membered bicyclic heterocyclic ring having 1-3 heteroatoms independently selected from nitrogen, oxygen, and sulfur;
R⁵, R¹⁰, and R^{10′} are each, independently, H, D, halo, oxo, C₁₋₆ alkyl, C₂₋₆ alkenyl, C₂₋₆ alkynyl, C₁₋₆ haloalkyl, C₃₋₆ cycloalkyl, CN, NO₂, C(O)NR₂, C(O)OR, S(O)OR, SO₂OR, S(O)NR₂, SO₂NR₂, or B(OR)₂;
optionally, R¹⁰ and R^{10′} together with the carbon atom to which they are both attached form an optionally substituted C₃₋₇ spirocyclic ring;
n is 0-8;
L² is an optionally substituted bivalent C₁₋₈ saturated or unsaturated, straight or branched hydrocarbon chain, wherein 1, 2, or 3 methylene units of the hydrocarbon chain are optionally and independently replaced by —NR—C(O)—, —C(O)—NR—, —C(O)—, —S(O)₂—, —C(O)—O—, —O—C(O)—, —NR—S(O)₂—, —S(O)₂—NR—, or -Cy-;
Cy- is an optionally substituted bivalent ring selected from phenyl, a 4-6 membered monocyclic heterocyclic ring having 1-4 heteroatoms independently selected from nitrogen, oxygen, and sulfur, a 5-10 membered bicyclic carbocyclic ring, and a 4-6 membered monocyclic heteroaromatic ring having 1-4 heteroatoms independently selected from nitrogen, oxygen, and sulfur;
R² is R¹⁴(CR¹⁵R^{15′})ₚ—;
R¹⁴ is H, D, —OH, —NR₂, —O(C₁₋₆ alkyl), —O(C₁₋₆ alkyl)-(optionally substituted 4-7 membered monocyclic heterocyclic ring), an optionally substituted C₁₋₆ alkyl, or an optionally substituted ring selected from a 3-7 membered monocyclic carbocyclic ring, a 4-7 membered monocyclic heterocyclic ring having 1-4 heteroatoms independently selected from nitrogen, oxygen, and sulfur, a 5-10 membered bicyclic carbocyclic ring, a 7-10 membered bicyclic heterocyclic ring having 1-4 heteroatoms independently selected from nitrogen, oxygen, and sulfur, phenyl, 5-6 membered monocyclic heteroaromatic ring having 1-4 heteroatoms independently selected from nitrogen, oxygen, and sulfur, a 8-10 membered bicyclic aromatic ring, a 8-10 membered bicyclic heteroaromatic ring having 1-4 heteroatoms independently selected from nitrogen, oxygen, and sulfur, and adamantyl;
R¹⁵, and R^{15′} are each, independently, H, D, halo, oxo, C₁₋₆ alkyl, C₂₋₆ alkenyl, C₂₋₆ alkynyl, C₁₋₆ haloalkyl, C₃₋₆ cycloalkyl, CN, NO₂, C(O)NR₂, C(O)OR, or B(OR)₂;
p is 0-8;
R³ is absent, hydrogen, —OH, halogen, —CN, —C(O) H, —NH₂, —NO₂, —COOH, —CONH₂, —NH—C(O)—O—C₁₋₆ alkyl, C₁₋₆ alkyl, or —C(O)—C₁₋₆ alkyl, wherein the C₁₋₆ alkyl is optionally substituted;
L³ is a bond, or an optionally substituted bivalent C₁₋₈ saturated or unsaturated, straight or branched hydrocarbon chain, wherein 1, 2, or 3 methylene units of the hydrocarbon chain are optionally replaced by —NR—C(O)—, —C(O)—NR, —C(O)—, —S(O)₂—, —C(O)—O—, —O—C(O)—, —NR—S(O)₂—, or —S(O)₂—NR—;
R⁴ is —NH₂, NHR, —N═C═N—, —NR₂, —CH₂—NH—C≡N, —PO₄H₂, or —H; and
each R is, independently, H, —OH, —C₁₋₈ alkyl, —C₁₋₈ alkyl(3-7 membered monocyclic carbocyclyl), —OC₁₋₈ alkyl, —C(O)—C₁₋₈ alkyl, —O—C(O)—C₁₋₈ alkyl, C₁₋₈ alkyl-O—C(O)—C₁₋₈ alkyl, —C(O)—OC₁₋₈ alkyl, 3-7 membered monocyclic carbocyclyl, —O-(4-7 membered monocyclic carbocyclyl), —C(O)-(4-7 membered monocyclic carbocyclyl), —C(O)—O-(4-7 membered monocyclic carbocyclyl), phenyl, —O-phenyl, —C(O)-phenyl, —C(O)—O-phenyl, —C(O)-phenyl-(C₁₋₈ alkyl)-O—C(O)—O—(C₁₋₈ alkyl)-, —C(O)—O—(C₁₋₈ alkyl)-(4-7 membered monocyclic heterocyclic ring), 8-10 membered bicyclic aryl, —O-(8-10 membered bicyclic aryl), —C(O)-(8-10 membered bicyclic aryl), or —C(O)—O-(8-10 membered bicyclic aryl), wherein each of the C₁₋₈ alkyl, 4-7 membered monocyclic carbocyclyl, phenyl, and 8-10 membered bicyclic aryl is optionally and independently substituted,
with the proviso that the compound is not: 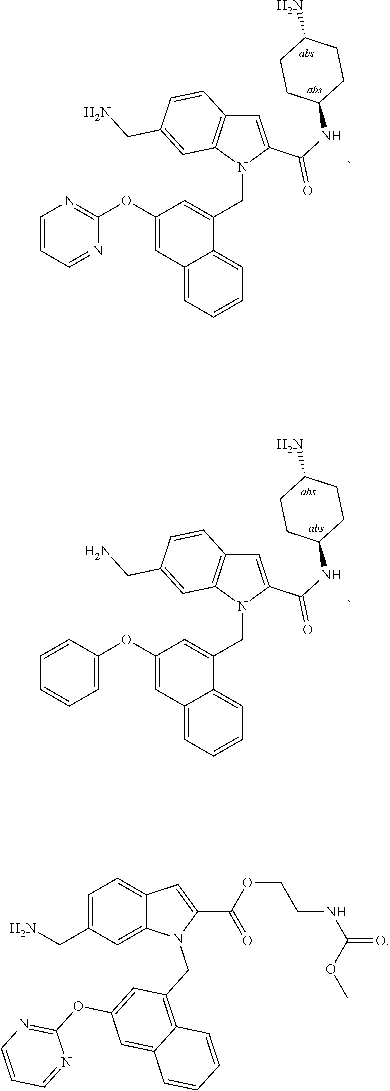 [SMILES: NCc1ccc2cc(C(=O)N[C@H]3CC[C@H](N)CC3)n(Cc3cc(Oc4ncccn4)cc4ccccc34)c2c1 ; NCc1ccc2cc(C(=O)N[C@H]3CC[C@H](N)CC3)n(Cc3cc(Oc4ccccc4)cc4ccccc34)c2c1 ; COC(=O)NCCOC(=O)c1cc2ccc(CN)cc2n1Cc1cc(Oc2ncccn2)cc2ccccc12]
or a pharmaceutically acceptable salt thereof.

2. The compound of claim 1, or a pharmaceutically acceptable salt thereof, wherein the compound has a formula of: 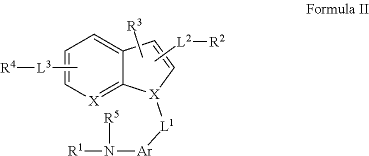
or a pharmaceutically acceptable salt thereof.

3. The compound of claim 1, or a pharmaceutically acceptable salt thereof, wherein the compound is of Formula III-a or III-b: 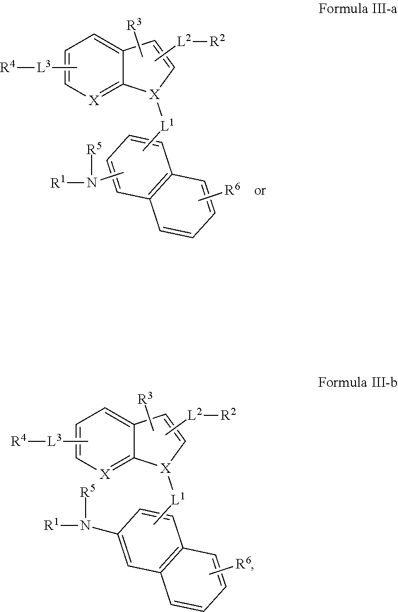
or a pharmaceutically acceptable salt thereof, wherein R⁶ is absent, —OH, halogen, —CN, —C(O)H, —NH₂, —NO₂, —COOH, —CONH₂, —CONHR, —NH—C(O)—O—C₁₋₆ alkyl, C₁₋₆ alkyl, or —C(O)—C₁₋₆ alkyl, wherein the C₁₋₆ alkyl is optionally substituted

4. The compound of claim 1, or a pharmaceutically acceptable salt thereof, wherein the compound is of Formula IV: 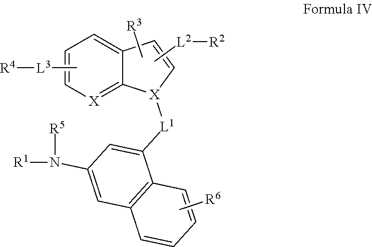
or a pharmaceutically acceptable salt thereof.

5. The compound of claim 1, or a pharmaceutically acceptable salt thereof, wherein the compound is of Formula V: 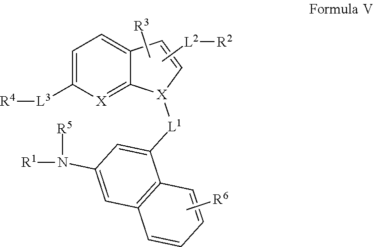
or a pharmaceutically acceptable salt thereof.

6. The compound of claim 1, or a pharmaceutically acceptable salt thereof, wherein the compound is of Formula VI: 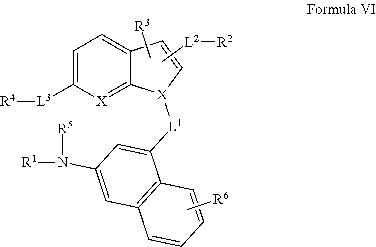
or a pharmaceutically acceptable salt thereof.

7. The compound of claim 1, or a pharmaceutically acceptable salt thereof, wherein the compound is of Formula VII: 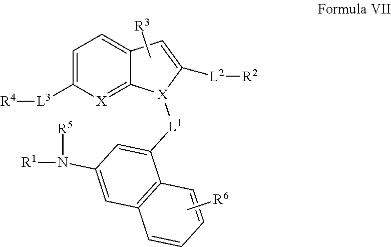
or a pharmaceutically acceptable salt thereof.

8. The compound of any one of claims 1-7, or a pharmaceutically acceptable salt thereof, wherein L¹ is —CH₂—.

9. The compound of any one of claims 1-8, or a pharmaceutically acceptable salt thereof, wherein L² is —C(O)— or —CH₂CH₂—.

10. The compound of any one of claims 1-9, or a pharmaceutically acceptable salt thereof, wherein L³ is —CH₂—.

11. The compound of any one of claims 1-10, or a pharmaceutically acceptable salt thereof, wherein R⁴ is —NH₂.

12. The compound of any one of claims 1-11, or a pharmaceutically acceptable salt thereof, wherein R⁵ is H.

13. The compound of claim 12, or a pharmaceutically acceptable salt thereof, wherein the compound has a formula of: 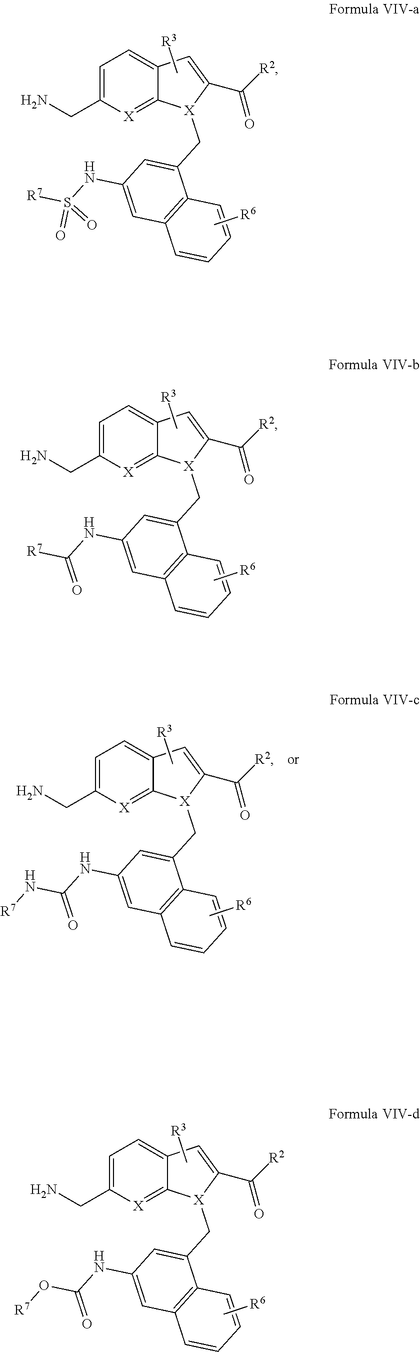
or a pharmaceutically acceptable salt thereof.

14. The compound of claim 12, or a pharmaceutically acceptable salt thereof, wherein the compound is of Formula IX: 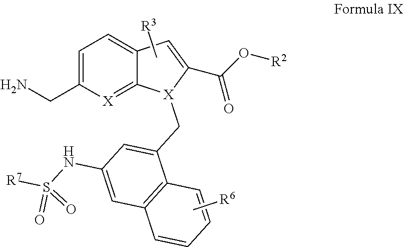
or a pharmaceutically acceptable salt thereof.

15. The compound of claim 12, or a pharmaceutically acceptable salt thereof, wherein the compound is of Formula X: 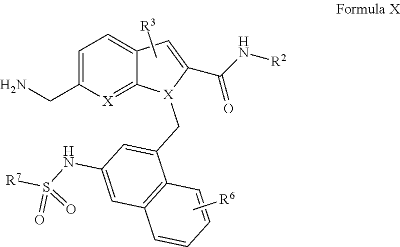
or a pharmaceutically acceptable salt thereof.

16. The compound of any one of claims 2-15, or a pharmaceutically acceptable salt thereof, wherein four instances of X are CH, one instance of X is C-L³-R⁴, one instance of X is C-L²-R², and one instance of X is N.

17. The compound of any one of claims 2-15, or a pharmaceutically acceptable salt thereof, wherein three instances of X are CH, one instance of X is C-L³-R⁴, one instance of X is C-L²-R², and two instances of X are N.

18. The compound according to claim 16 or 17, or a pharmaceutically acceptable salt thereof, wherein R³ is H.

19. The compound of any one of claims 2-18, or a pharmaceutically acceptable salt thereof, wherein R⁶ is absent.

20. The compound of any one of claims 13-19, or a pharmaceutically acceptable salt thereof, wherein R⁷ is R¹¹(CH₂)ₙ—.

21. The compound claim 20, or a pharmaceutically acceptable salt thereof, wherein n is 0.

22. The compound claim 20, or a pharmaceutically acceptable salt thereof, wherein n is 1.

23. The compound claim 20, or a pharmaceutically acceptable salt thereof, wherein n is 2.

24. The compound of any one of claims 20-23, or a pharmaceutically acceptable salt thereof, wherein R¹¹ is: 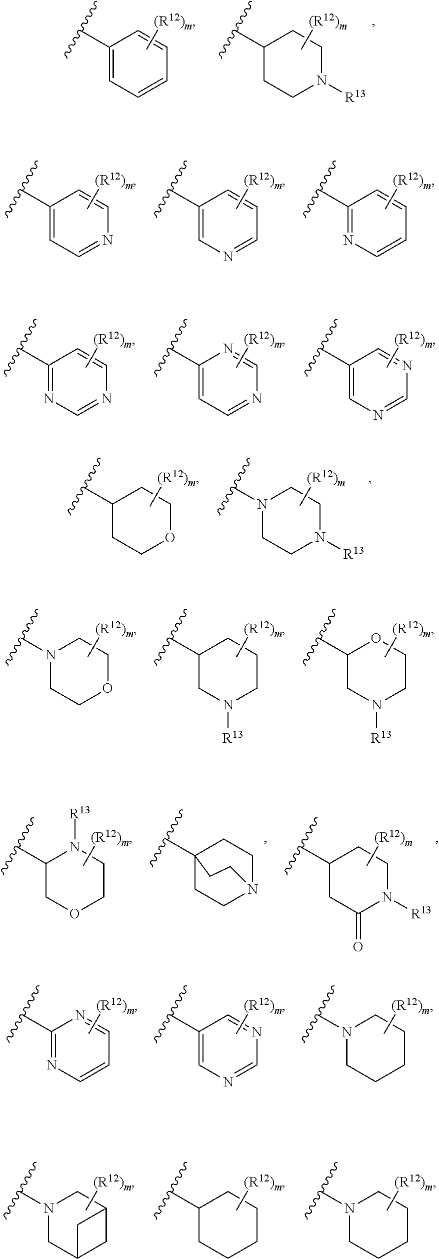 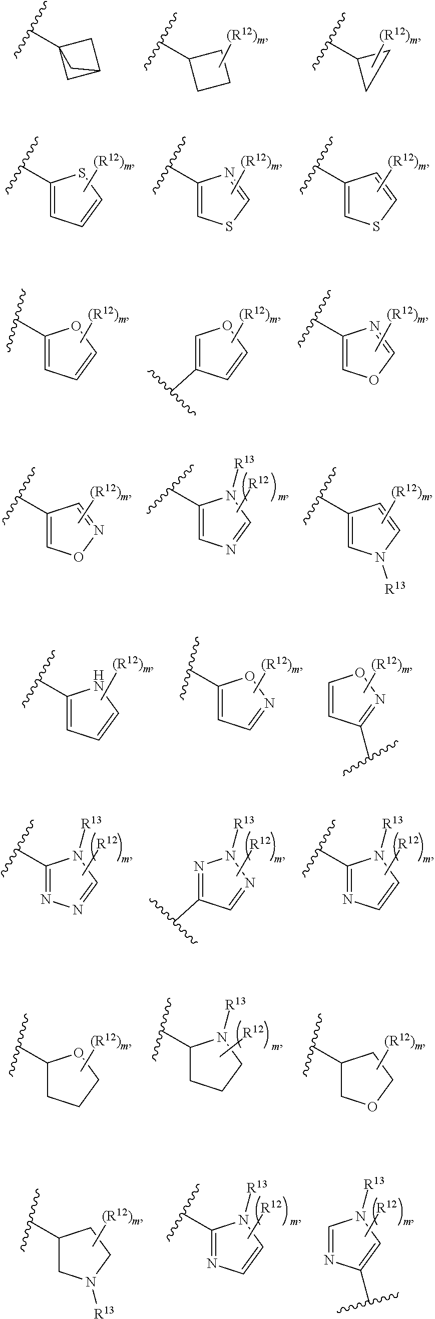 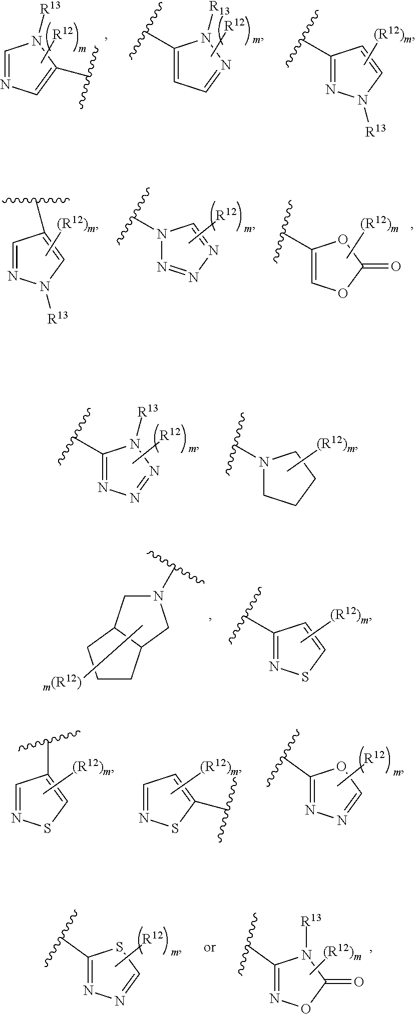
wherein m is 0-5 and R¹² and R¹⁵ are each, independently, hydrogen, —OH, halogen, —CN, —C(O) H, —NH₂, —NHR, —NR₂, —NO₂, —COOH, —CONH₂, —NH—C(O)—O—C₁₋₆ alkyl, C₁₋₆ alkyl, —(O)—C₁₋₆ alkyl, haloalkyl, ═O, —(C₁₋₆ alkyl)-O—(C₁₋₆ alkyl), —(C₁₋₆ alkyl)-O—(C₁₋₆ alkyl)-O—(C₁₋₆ alkyl), —C(O)NH—C₁₋₆ alkyl, —C(O)—C₁₋₆ alkyl, —O—C(O)—C₁₋₆ alkyl, —C(O)—OC₁₋₆ alkyl, —CH═CH₂, phenyl, —S(═O)₂ (C₁₋₆ alkyl), or —S(═O)₂(carbocyclic), wherein the C₁₋₆ alkyl is optionally substituted.

25. The compound of any one of claims 13-24, or a pharmaceutically acceptable salt thereof, wherein R² is R¹⁴(CH₂)ₚ—.

26. The compound of claim 25, or a pharmaceutically acceptable salt thereof, wherein p is 0.

27. The compound of claim 25, or a pharmaceutically acceptable salt thereof, wherein p is 1.

28. The compound of claim 25, or a pharmaceutically acceptable salt thereof, wherein p is 0-4.

29. The compound of any one of claims 25-28, or a pharmaceutically acceptable salt thereof, wherein R¹⁴ is H.

30. The compound of any one of claims 25-28, or a pharmaceutically acceptable salt thereof, wherein R¹⁴ is: 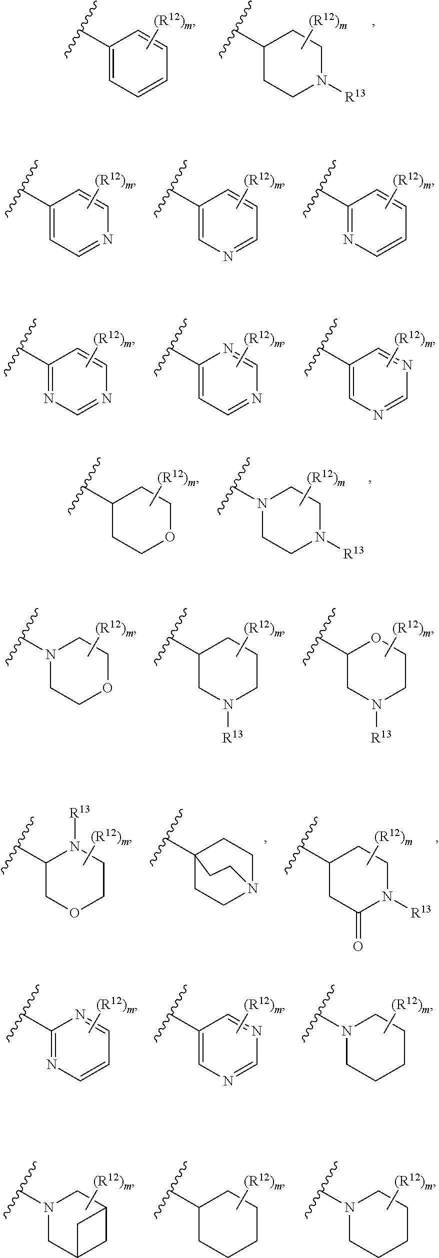 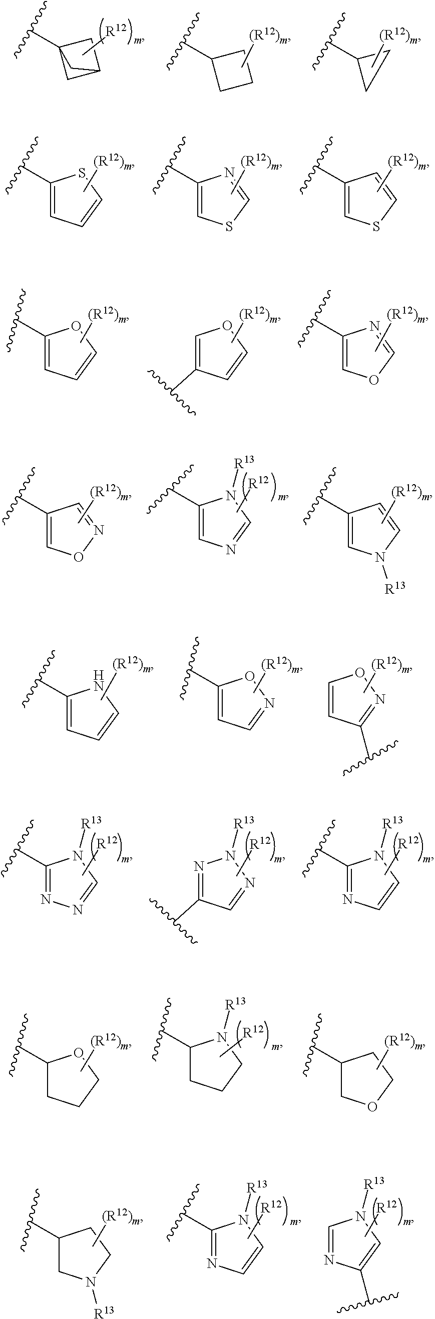 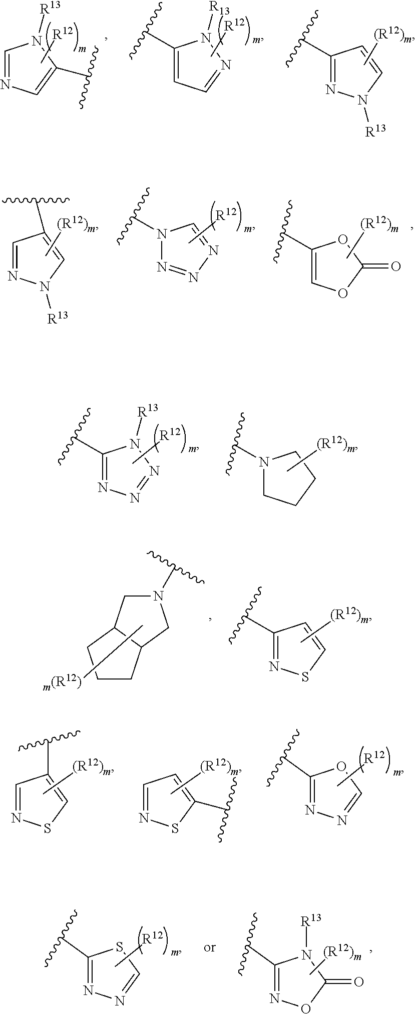
wherein m is 0-5 and R¹² and R¹³ are each, independently, H, —OH, halogen, —CN, —C(O) H, —NH₂, —NHR, —NR₂, —NO₂, —COOH, —CONH₂, —NH—C(O)—O—C₁₋₆ alkyl, C₁₋₆ alkyl, —(O)—C₁₋₆ alkyl, haloalkyl, ═O, —(C₁₋₆ alkyl)-O—(C₁₋₆ alkyl), —(C₁₋₆ alkyl)-O—(C₁₋₆ alkyl)-O—(C₁₋₆ alkyl), —C(O)NH—C₁₋₆ alkyl, —C(O)—C₁₋₆ alkyl, —O—C(O)—C₁₋₆ alkyl, —C(O)—OC₁₋₆ alkyl, —CH═CH₂, phenyl, —S(═O)₂(C₁₋₆ alkyl), —S(═O)₂(carbocyclic ring), or a 3-7 membered monocyclic carbocyclic ring, wherein the C₁₋₆ alkyl is optionally substituted.

31. A compound selected from those depicted in Table A, Table B, or Table C or a pharmaceutically acceptable salt thereof.

32. A pharmaceutical composition comprising the compound of any one of claims 1-31, or a pharmaceutically acceptable salt thereof, and a pharmaceutically acceptable excipient.

33. A pharmaceutical composition comprising the compound of any one of claims 1-31, or a pharmaceutically acceptable salt thereof, and a pharmaceutically acceptable carrier, adjuvant, or vehicle.

34. A method for treating a low hepcidin disorder, disease, and/or condition in a subject, comprising administering to the subject the compound of any one of claims 1-31, or a pharmaceutically acceptable salt thereof, or the pharmaceutical composition of any one of claims 32-33.

35. A method for increasing hepcidin production by the liver in a subject, comprising administering to the subject the compound of any one of claims 1-31, or a pharmaceutically acceptable salt thereof, or the pharmaceutical composition of any one of claims 32-33.

36. A method for treating an iron overload disorder, disease, and/or condition in a subject, comprising administering to the subject the compound of any one of claims 1-31, or a pharmaceutically acceptable salt thereof, or the pharmaceutical composition of any one of claims 32-33.

37. The method of claim 36, wherein the iron overload disorder, disease, and/or condition is selected from the group consisting of hemochromatosis Type 1, 2a, 2b, and 3 (hemochromatosis, Hfe hemochromatosis (Type 1), juvenile hemochromatosis (types 2a and 2b), hepcidin deficiency, transfusional iron overload, African iron overload, and iron overload cardiomyopathy.

38. A method for treating an iron loading anemia in a subject, comprising administering to the subject the compound of any one of claims 1-31, or a pharmaceutically acceptable salt thereof, or the pharmaceutical composition of any one of claims 32-33.

39. The method of claim 38, wherein the iron loading anemia is selected from the group consisting of beta thalassemia, HbE/thalassemia (thalassemia major, thalassemia intermedia, thalassemia minor, non-transfusion dependent thalassemia, transfusion-dependent thalassemia), alpha thalassemia congenital dyserythropoietic anemias (Type I and Type II), pyruvate kinase deficiency, and myelodysplasia (such as myelodysplastic syndrome, and RARS SF3B1 associated MDS).

40. A method for treating a hematological disease, disorder, and/or condition in a subject, comprising administering to the subject the compound of any one of claims 1-31, or a pharmaceutically acceptable salt thereof, or the pharmaceutical composition of any one of claims 32-33.

41. The method of claim 40, wherein the hematological disease, disorder, and/or condition is selected from the group consisting of sickle cell disease (such as sickle cell anemia), polycythemia vera, sideroblastic anemia, and bone marrow transplantation.

42. A method for treating a liver disease in a subject, comprising administering to the subject the compound of any one of claims 1-31, or a pharmaceutically acceptable salt thereof, or the pharmaceutical composition of any one of claims 32-33.

43. The method of claim 42, wherein the liver disease is selected from the group consisting of Hepatitis B, Hepatitis C, alcoholic liver disease, cirrhosis of the liver, hepatocellular carcinoma, and non-alcoholic steatohepatitis (NASH).

44. A method of treating a metabolic disease in a subject, comprising administering to the subject the compound of any one of claims 1-31, or a pharmaceutically acceptable salt thereof, or the pharmaceutical composition of any one of claims 32-33.

45. The method of claim 44, wherein the metabolic disease is selected from the group consisting of metabolic syndrome, insulin resistance, Type II diabetes, porphyria, porphyria cutanea tarda, Wilson's Disease, and acute iron overdose.

46. A method for treating a neurodegenerative disorder in a subject, comprising administering to the subject the compound of any one of claims 1-31, or a pharmaceutically acceptable salt thereof, or the pharmaceutical composition of any one of claims 32-33.

47. A method for treating an infectious disease in a subject, comprising administering to the subject the compound of any one of claims 1-31, or a pharmaceutically acceptable salt thereof, or the pharmaceutical composition of any one of claims 32-33.

48. The method of claim 47, wherein the infectious disease is a siderophilic infection.

49. A method for treating polycythemia vera in a subject, comprising administering to the subject the compound of any one of claims 1-31, or a pharmaceutically acceptable salt thereof, or the pharmaceutical composition of any one of claims 32-33.

50. The method of any one of claims 28-49, wherein the subject is a subject in need thereof.

51. A method of inhibiting matriptase 2, or a mutant thereof, in a biological sample, comprising contacting the sample with the compound of any one of claims 1-31, or the pharmaceutical composition of any one of claims 32-33.

52. A method of inhibiting matriptase 2, or a mutant thereof, in a subject, comprising administering to the subject the compound of any one of claims 1-31, or a pharmaceutically acceptable salt thereof, or the pharmaceutical composition of any one of claims 32-33.

53. The method of claim 51 or 52, wherein contacting is in vivo.

54. The method of claim 51 or 52, wherein contacting is in vitro.
